(19) United States
(12) Patent Application Publication
(10) Pub. No.: US 2024/0114777 A1
(43) Pub. Date: Apr. 4, 2024

(54) PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: So-Young JUNG, Gyeonggi-do (KR); Hyo-Nim SHIN, Gyeonggi-do (KR); Doo-Hyeon MOON, Gyeonggi-do (KR); Sang-Hee CHO, Gyeonggi-do (KR); Hyun-Ju KANG, Gyeonggi-do (KR)

(21) Appl. No.: 18/355,537

(22) Filed: Jul. 20, 2023

(30) Foreign Application Priority Data
Aug. 9, 2022 (KR) 10-2022-0099113
Jul. 13, 2023 (KR) 10-2023-0090920

(51) Int. Cl. H10K 85/60; C07D 307/91; C07D 487/04

(52) U.S. Cl. CPC H10K 85/636; C07D 307/91; C07D 487/04; H10K 85/615; H10K 85/626; H10K 85/654; H10K 85/6574; H10K 50/11; H10K 85/6572

(57) ABSTRACT

The present disclosure relates to a plurality of host materials, an organic electroluminescent compound, and an organic electroluminescent device comprising the same. By comprising a specific combination of compounds according to the present disclosure as a plurality of host materials or by comprising the compound according to the present disclosure, it is possible to produce an organic electroluminescent device having lower driving voltage, higher luminous efficiency, and/or longer lifetime properties compared to the conventional organic electroluminescent devices.

### TECHNICAL FIELD

The present disclosure relates to a plurality of host materials, an organic electroluminescent compound, and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation.

However, in many applications such as TVs and lightings, the lifetime of OLEDs is insufficient and higher efficiency of OLEDs is still required. Typically, the higher the luminance of an OLED, the shorter the lifetime that the OLED has. Therefore, an OLED having long lifetime characteristics is required for long time use and high resolution of a display.

Meanwhile, Korean Patent Publication No. 1612164 discloses a compound in which an amine group is fused to a dibenzo moiety comprising a carbazole, a fluorene or a 5-membered hetero ring to form a ring. However, the aforementioned reference does not specifically disclose a plurality of host materials comprising a specific compound or a specific combination of host materials, claimed in the present disclosure. In addition, it is continuously required to develop a light-emitting material having more improved performances, for example, improved driving voltage, luminous efficiency and/or lifetime properties, as compared with the previously disclosed OLEDs.

### DISCLOSURE OF INVENTION

### Technical Problem

The objective of the present disclosure is to provide a plurality of host materials capable of providing an organic electroluminescent device having improved driving voltage, luminous efficiency and/or lifetime properties. Another objective of the present disclosure is to provide an organic electroluminescent compound having a new structure suitable for applying to an organic electroluminescent device. Still another objective of the present disclosure is to provide an organic electroluminescent device having significantly improved driving voltage, luminous efficiency and/or lifetime properties by comprising a compound or a specific combination of compounds of the present disclosure.

### Solution to Problem

As a result of intensive studies to solve the technical problems, the present inventors found that the above objective can be achieved by a plurality of host materials comprising at least one first host compound represented by the following formula 1 and at least one second host compound represented by the following formula 2, or a compound represented by the following formula 1′ or any one of the following formulas 2′-1 to 2′-4.

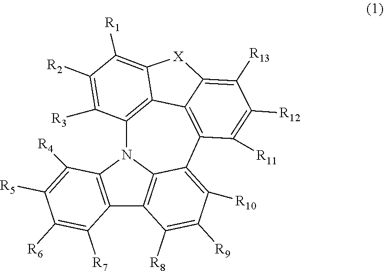

In formula 1,
X represents —O—, —S—, —N(R)—, C(R′)(R″)— or —Se—;
R, R′, and R″, each independently, represent a substituted or unsubstituted (C1-C30)alkyl, a substituted or unsubstituted (C6-C30)aryl, or a substituted or unsubstituted (3- to 30-membered)heteroaryl, -L₁-N—(Ar₁)(Ar₂) or -L₂-N(Ar₃)-L₃-N—(Ar₄)(Ar₅); or R′ and R″ may be linked to each other to form a ring(s), and R′ and R″ may be the same or different;
R₁ to R₁₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), -L₁-N—(Ar₁)(Ar₂) or -L₂-N(Ar₃)-L₃-N—(Ar₄)(Ar₅); or may be linked to an adjacent substituent(s) to form a ring(s);
with the proviso that at least one of R, R′, R″, and R₁ to R₁₃ represents -L₁-N—(Ar₁)(Ar₂) or -L₂-N(Ar₃)-L₃-N—(Ar₄)(Ar₅);
L₁ and L₂, each independently, represent a single bond, a substituted or unsubstituted (C6-C30)arylene, or a substituted or unsubstituted (3- to 30-membered)heteroarylene;
L₃ represents a substituted or unsubstituted (C6-C30)arylene, or a substituted or unsubstituted (3- to 30-membered)heteroarylene; and
Ar₁ to Ar₅, each independently, represent a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl.

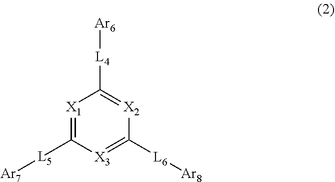

In formula 2,
X₁ to X₃, each independently, represent —N═ or —C(R₂₀)═, with the proviso that at least one of X₁ to X₃ represents N;
R₂₀ represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s);
L₄ to L₆, each independently, represent a single bond, a substituted or unsubstituted (C6-C30)arylene, a substituted or unsubstituted (C3-C30)cycloalkylene, or a substituted or unsubstituted (3- to 30-membered)heteroarylene;
Ar₆ to Ar₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or *—N—(R₂₁)(R₂₂); or may be linked to an adjacent substituent(s) to form a ring(s); with the proviso that at least one of Ar₆ to Ar₈ represents a substituted or unsubstituted (C6-C30)aryl or a substituted or unsubstituted (3- to 30-membered)heteroaryl; and
R₂₁ and R₂₂, each independently, represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

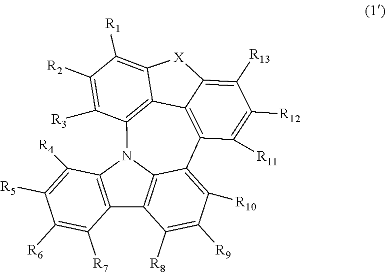

In formula 1′,
X represents —N(R)—, —C(R′)(R″)— or —Se—;
R, R′, and R″, each independently, represent a substituted or unsubstituted (C1-C30)alkyl, a substituted or unsubstituted (C6-C30)aryl, or a substituted or unsubstituted (3- to 30-membered)heteroaryl, -L₁-N—(Ar₁)(Ar₂) or -L₂-N(Ar₃)-L₃-N—(Ar₄)(Ar₅); or R′ and R″ may be linked to each other to form a ring(s), and R′ and R″ may be the same or different;
R₁ to R₁₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), -L₁-N—(Ar₁)(Ar₂) or -L₂-N(Ar₃)-L₃-N—(Ar₄)(Ar₅); or may be linked to an adjacent substituent(s) to form a ring(s);
with the proviso that at least one of R, R′, R″, and R₁ to R₁₃ represents -L₁-N—(Ar₁)(Ar₂) or -L₂-N(Ar₃)-L₃-N—(Ar₄)(Ar₅);
L₁ and L₂, each independently, represent a single bond, a substituted or unsubstituted (C6-C30)arylene, or a substituted or unsubstituted (3- to 30-membered)heteroarylene;
L₃ represents a substituted or unsubstituted (C6-C30)arylene, or a substituted or unsubstituted (3- to 30-membered)heteroarylene; and
Ar₁ to Ar₅, each independently, represent a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl.

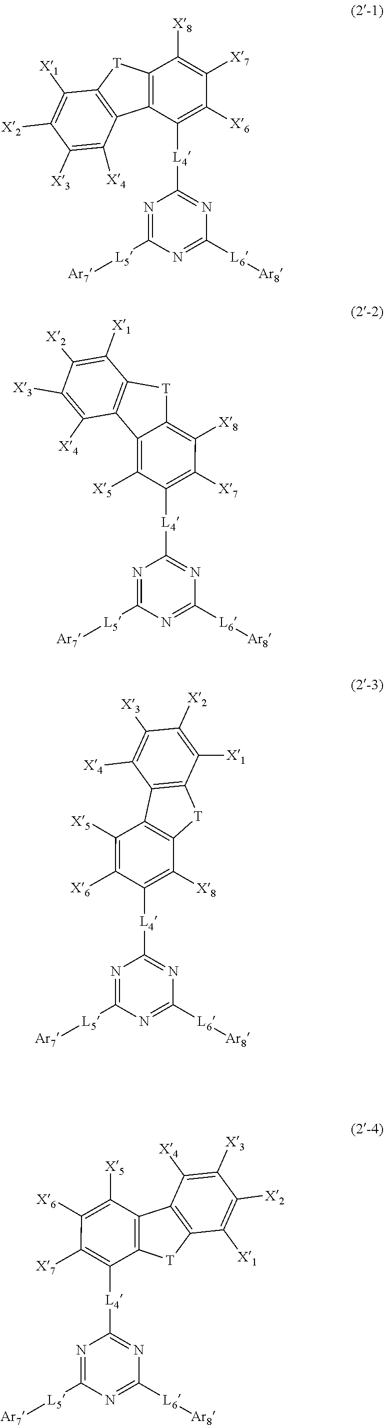

In formulas 2′-1 to 2′-4,
T represents O or S;
X₁′ to X₄′, each independently, represent hydrogen, deuterium, a substituted or unsubstituted (C1-C30)alkyl, a substituted or unsubstituted (C6-C30)aryl, a substituted or unsubstituted (3- to 30-membered)heteroaryl;
L₄′ to L₆′, each independently, represent a single bond, a phenylene unsubstituted or substituted with deuterium, or a naphthylene unsubstituted or substituted with deuterium; and
Ar₇′ and Ar₈′, each independently, represent a phenyl unsubstituted or substituted with deuterium, a biphenyl unsubstituted or substituted with deuterium, a naphthyl unsubstituted or substituted with deuterium, a substituted or unsubstituted pyridyl, a substituted or unsubstituted dibenzofuranyl, a substituted or unsubstituted dibenzothiophenyl, a substituted or unsubstituted benzonaphthofuranyl, or a substituted or unsubstituted benzonaphthothiophenyl;
in formula 2′-1,
X₆′ to X₈′, each independently, represent hydrogen, deuterium or -L₇-Ar₉, with the proviso that at least one of X₆′ to X₈′ represents -L₇-Ar₉;
if X₆′ is -L₇-Ar₉, L₇ represents a single bond, a phenylene unsubstituted or substituted with deuterium, or a naphthylene unsubstituted or substituted with deuterium, and Ar₉ represents a phenyl unsubstituted or substituted with deuterium, a biphenyl unsubstituted or substituted with deuterium, or a naphthyl unsubstituted or substituted with deuterium; with the proviso that if L₇ is a single bond, Ar₉ represents a naphthyl unsubstituted or substituted with deuterium;
if X₇′ is -L₇-Ar₉, L₇ represents a single bond, a phenylene unsubstituted or substituted with deuterium, or a naphthylene unsubstituted or substituted with deuterium, Ar₉ represents a phenyl unsubstituted or substituted with deuterium, a biphenyl unsubstituted or substituted with deuterium, or a naphthyl unsubstituted or substituted with deuterium;
if X₈′ is -L₇-Ar₉, L₇ represents a single bond, a phenylene unsubstituted or substituted with deuterium, or a naphthylene unsubstituted or substituted with deuterium, Ar₉ represents a phenyl unsubstituted or substituted with deuterium, or a naphthyl unsubstituted or substituted with deuterium; and
with the proviso that if X₈′ is -L₇-Ar₉ and a phenyl, at least one of Ar₇′ and Ar₈′ represents a dibenzofuranyl or a dibenzothiophenyl;
in formula 2′-2,
X₆′ represents -L₇-Ar₉, and X₅′ and X₇′, each independently, represent hydrogen or deuterium;
L₇ represents a single bond, a phenylene unsubstituted or substituted with deuterium, or a naphthylene unsubstituted or substituted with deuterium, and Ar₉ represents hydrogen, a phenyl unsubstituted or substituted with deuterium, a biphenyl unsubstituted or substituted with deuterium, or a naphthyl unsubstituted or substituted with deuterium or a phenyl(s); and
with the proviso that if L₇ is a single bond, Ar₉ represents a naphthyl unsubstituted or substituted with deuterium;
in formula 2′-3,
X₅′, X₆′ and X₈′, each independently, represent hydrogen, deuterium, or -L₇-Ar₉, with the proviso that at least one of X₅′, X₆′ and X₈′ represents -L₇-Ar₉;
L₇ represents a single bond, a phenylene unsubstituted or substituted with deuterium, or a naphthylene unsubstituted or substituted with deuterium, Ar₉ represents a phenyl unsubstituted or substituted with deuterium or a naphthyl(s), a biphenyl unsubstituted or substituted with deuterium, or a naphthyl unsubstituted or substituted with deuterium; and
with the proviso that if X₅′ is -L₇-Ar₉ and a naphthyl, at least one of Ar₇′ and Ar₈′ represents a substituted or unsubstituted naphthyl, a substituted or unsubstituted dibenzofuranyl, or a substituted or unsubstituted dibenzothiophenyl; in formula 2′-4,
X₅′ to X₇, each independently, represent hydrogen, deuterium, or -L₇-Ar₉, with the proviso that at least one of X₅′ to X₇′ represents -L₇-Ar₉; and
L₇ represents a single bond, a phenylene unsubstituted or substituted with deuterium, or a naphthylene unsubstituted or substituted with deuterium, and Ar₉ represents a phenyl unsubstituted or substituted with deuterium, a biphenyl unsubstituted or substituted with deuterium, or a naphthyl unsubstituted or substituted with deuterium.

### Advantageous Effects of Invention

An organic electroluminescent device having significantly improved driving voltage, luminous efficiency and/or lifetime properties compared to conventional organic electroluminescent devices can be produced by comprising a specific combination of compounds according to the present disclosure as a plurality of host materials, or by comprising a compound according to the present disclosure.

### MODE FOR THE INVENTION

Hereinafter, the present disclosure will be described in detail. However, the following description is intended to explain the present disclosure and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of host materials” in the present disclosure means a host material comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, the plurality of host materials of the present disclosure is a combination of at least two host materials, and may selectively further comprise conventional materials comprised in an organic electroluminescent material. At least two compounds comprised in the plurality of host materials of the present disclosure may be comprised together in one light-emitting layer or may respectively be comprised in different light-emitting layers. For example, the at least two host materials may be mixture-evaporated or co-evaporated, or may be individually evaporated.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. The term “(C3-C30)cycloalkyl(ene)” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7 ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms. The above aryl may be partially saturated, and may comprise a spiro structure. The above aryl may include phenyl, biphenyl, terphenyl, quaterphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluoren]yl, azulenyl, tetramethyldihydrophenanthrenyl, cumenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” is meant to be an aryl(ene) group having 3 to 30 ring backbone atoms, and including at least one, preferably 1 to 4 heteroatoms selected from the group consisting of B, N, O, S, Si, P, Se, and Ge. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolinyl, benzofuroquinazolinyl, benzofuronaphthyridinyl, benzofuropyrimidinyl, naphthofuropyrimidinyl, benzothienoquinolyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, benzocarbazolyl, dibenzocarbazolyl, phenoxazinyl, phenanthridinyl, benzodioxolyl, dihydroacridinyl, benzotriazolyl, phenazinyl, imidazopyridyl, chromenoquinazolinyl, thiochromenoquinazolinyl, dimethylbenzopyrimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, if two substituents in a benzene derivative occupy positions 1 and 2, it is called an ortho position. Meta indicates that two substituents are at positions 1 and 3, and for example, if two substituents in a benzene derivative occupy positions 1 and 3, it is called a meta position. Para indicates that two substituents are at positions 1 and 4, and for example, if two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as one heteroaryl substituent, or as substituents in which two heteroaryl substituents are linked. Herein, the substituent(s) of the substituted alkyl, the substituted alkenyl, the substituted aryl, the substituted arylene, the substituted heteroaryl, the substituted heteroarylene, the substituted cycloalkyl, the substituted cycloalkylene, the substituted alkoxy, the substituted trialkylsilyl, the substituted dialkylarylsilyl, the substituted alkyldiarylsilyl, the substituted triarylsilyl, and the substituted fused ring group of a aliphatic ring(s) and a aromatic ring(s), each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl unsubstituted or substituted with a (C6-C30)aryl(s); a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30) arylthio; a (3- to 30-membered)heteroaryl unsubstituted or substituted with a (C6-C30)aryl(s); a (C6-C30)aryl unsubstituted or substituted with at least one of a (C1-C30)alkyl(s) and (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a substituted or unsubstituted mono- or di-(C6-C30)arylamino; a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl, in which the substituents may be further substituted with deuterium. According to one embodiment of the present disclosure, the substituent(s), each independently, are at least one selected from the group consisting of deuterium; a cyano; a (C1-C20)alkyl; a (C6-C25) aryl; a (5- to 25-membered)heteroaryl; a substituted or unsubstituted di(C6-C25)arylamino; and a (C6-C25)aryl(C1-C20)alkyl. According to another embodiment of the present disclosure, the substituent(s), each independently, are at least one selected from the group consisting of deuterium; a cyano; a (C1-C10)alkyl; a (C6-C20) aryl; a (5- to 20-membered)heteroaryl; a di(C6-C18)arylamino unsubstituted or substituted with deuterium; and a (C6-C18)aryl(C1-C10)alkyl. For example, the substituent(s) may be at least one selected from the group consisting of deuterium, a cyano, a methyl, a phenyl, a naphthyl, a biphenyl, a phenanthrenyl, a fluorenyl, a phenylfluorenyl, a cumenyl, an isopropyl substituted with a phenyl(s), a triphenylsilyl, a carbazolyl, a dibenzofuranyl, a dibenzothiophenyl, a phenylbiphenylamino, and a diphenylamino, in which the substituents may be further substituted with deuterium.

In the present disclosure, the term “a ring formed by a linkage of adjacent substituents” means that at least two adjacent substituents are linked to or fused with each other to form a substituted or unsubstituted, mono- or polycyclic, (3- to 30-membered) alicyclic or aromatic ring, or the combination thereof. The ring may preferably be a substituted or unsubstituted, mono- or polycyclic, (3- to 25-membered) alicyclic or aromatic ring, or the combination thereof, more preferably a mono- or polycyclic, (5- to 25-membered) aromatic ring unsubstituted or substituted with at least one of a (C6-C18)aryl(s) and a (5- to 25-membered)heteroaryl(s). In addition, the formed ring may contain at least one heteroatom selected from B, N, O, S, Si, P, Se, and Ge, preferably at least one heteroatom selected from N, O, S, and Se. For example, the ring may be a benzene ring, a cyclopentane ring, an indan ring, a fluorene ring unsubstituted or substituted with a phenyl(s), a phenanthrene ring, an indole ring, a xanthene ring, a benzofuropyridine ring, a benzothienopyridine ring, a dibenzothiophene ring, a dibenzofuran ring, a naphthalene ring, a benzofluorene ring, a benzothiophene ring, a benzofuran ring, an indene ring, a carbazole ring, etc.

In the present disclosure, heteroaryl(ene) and heterocycloalkyl, each independently, may comprise at least one heteroatom selected from B, N, O, S, Si, P, Se and Ge. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

The plurality of host materials of the present disclosure comprises a first host compound and a second host compound, wherein the first host material comprises at least one first host compound represented by formula 1, and the second host material comprises at least one second host compound represented by formula 2.

The first host compound, which is a host material according to one embodiment, may be represented by formula 1.

In formula 1, X represents —O—, —S—, —N(R)—, C(R′)(R″)— or —Se—. According to one embodiment of the present disclosure, X represents —N(R)—, C(R′)(R″)— or —Se—.

R, R′, and R″, each independently, represent a substituted or unsubstituted (C1-C30)alkyl, a substituted or unsubstituted (C6-C30)aryl, or a substituted or unsubstituted (3- to 30-membered)heteroaryl, -L₁-N—(Ar₁)(Ar₂) or -L₂-N(Ar₃)-L₃-N—(Ar₄)(Ar₅); or R′ and R″ may be linked to each other to form a ring(s), and R′ and R″ may be the same or different.

According to one embodiment of the present disclosure, R, R′, and R″, each independently, represent a substituted or unsubstituted (C1-C20)alkyl, -L₁-N—(Ar₁)(Ar₂) or -L₂-N(Ar₃)-L₃-N—(Ar₄)(Ar₅). According to another embodiment of the present disclosure, R, R′, and R″, each independently, represent an unsubstituted (C1-C10)alkyl, -L₁-N—(Ar₁)(Ar₂) or -L₂-N(Ar₃)-L₃-N—(Ar₄)(Ar₅). For example, R may be -L₁-N—(Ar₁)(Ar₂) or -L₂-N(Ar₃)-L₃-N—(Ar₄)(Ar₅), and R′ and R″ may be a methyl.

In formula 1, R₁ to R₁₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), -L₁-N—(Ar₁)(Ar₂) or -L₂-N(Ar₃)-L₃-N—(Ar₄)(Ar₅); or may be linked to an adjacent substituent(s) to form a ring(s). For example, R₁ to R₁₃, each independently, may be hydrogen, deuterium, -L₁-N—(Ar₁)(Ar₂) or -L₂-N(Ar₃)-L₃-N—(Ar₄)(Ar₅).

In formula 1, at least one of R, R′, R″, and R₁ to R₁₃ represent -L₁-N—(Ar₁)(Ar₂) or -L₂-N(Ar₃)-L₃-N—(Ar₄)(Ar₅). According to one embodiment of the present disclosure, any one or two of R, R′, R″, and R₁ to R₁₃, each independently, represent -L₁-N—(Ar₁)(Ar₂) or -L₂-N(Ar₃)-L₃-N—(Ar₄)(Ar₅).

In formula 1, L₁ and L₂, each independently, represent a single bond, a substituted or unsubstituted (C6-C30)arylene, or a substituted or unsubstituted (3- to 30-membered)heteroarylene. According to one embodiment of the present disclosure, L₁ and L₂, each independently, represent a single bond, a substituted or unsubstituted (C6-C25)arylene, or a substituted or unsubstituted (5- to 25-membered)heteroarylene. According to another embodiment of the present disclosure, L₁ and L₂, each independently, represent a single bond, a substituted or unsubstituted (C6-C18)arylene, or an unsubstituted (5- to 20-membered)heteroarylene. The substituent(s) of the substituted arylene and the substituted heteroarylene, each independently, may be at least one selected from the group consisting of deuterium, a (C6-C30)aryl, and a di(C6-C30)arylamino. For example, L₁ and L₂, each independently, may be a single bond, a substituted or unsubstituted phenylene, an unsubstituted naphthylene, an unsubstituted dibenzofuranylene, etc., in which the substituent(s) of the substituted phenylene may be at least one selected from the group consisting of deuterium, a phenyl, a naphthyl, a diphenylamino unsubstituted or substituted with deuterium, and a phenylbiphenylamino.

In formula 1, L₃ represents a substituted or unsubstituted (C6-C30)arylene, or a substituted or unsubstituted (3- to 30-membered)heteroarylene. According to one embodiment of the present disclosure, L₃ represents a substituted or unsubstituted (C6-C25)arylene, or a substituted or unsubstituted (5- to 25-membered)heteroarylene. According to another embodiment of the present disclosure, L₃ represents a (C6-C18)arylene unsubstituted or substituted with at least one of deuterium and a (C6-C30)aryl(s). For example, L₃ may be a phenylene unsubstituted or substituted with at least one of deuterium and a phenyl(s), etc.

In formula 1, Ar₁ to Ar₅, each independently, represent a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl. According to one embodiment of the present disclosure, Ar₁ to Ar₅, each independently, represent a substituted or unsubstituted (C6-C25)aryl, or a substituted or unsubstituted (5- to 25-membered)heteroaryl. According to another embodiment of the present disclosure, Ar₁ to Ar₅, each independently, represent a substituted or unsubstituted (C6-C18)aryl, or a (5- to 20-membered)heteroaryl unsubstituted or substituted with a (C6-C18)aryl(s). The substituent(s) of the substituted aryl may be at least one selected from the group consisting of deuterium, a (C1-C30)alkyl, a (C6-C30)aryl, a (3- to 30-membered)heteroaryl, and a (C6-C30)aryl(C1-C30)alkyl. Specifically, Ar₁ to Ar₅, each independently, may be a substituted or unsubstituted, phenyl, naphthyl, biphenyl, terphenyl, dibenzofuranyl, dibenzothiophenyl, fluorenyl, spirobifluorenyl, quaterphenyl, cumenyl, carbazolyl, phenanthrenyl, benzophenanthrenyl, chrysenyl, triphenylenyl, benzocarbazolyl, benzonaphthofuranyl, benzonaphthothiophenyl, benzofluorenyl, benzothiazolyl, benzoxazolyl, benzimidazolyl, naphthoxazolyl, benzonaphthoxazolyl, naphthothiazolyl, benzonaphthothiazolyl, or naphthoimidazolyl. For example, Ar₁ to Ar₅, each independently, may be a substituted or unsubstituted phenyl, a naphthyl, an o-biphenyl, a m-biphenyl, a p-biphenyl, a naphthylphenyl, a phenylnaphthyl, a phenanthrenyl, a dimethylfluorenyl, a diphenylfluorenyl, a spirobifluorenyl, an o-terphenyl, a m-terphenyl, an o-quaterphenyl, a dibenzofuranyl unsubstituted or substituted with a phenyl(s), a dibenzothiophenyl, a phenylcarbazolyl, a dibenzoselenophenyl, a benzonaphthofuranyl, etc., in which the substituent(s) of the substituted phenyl may be at least one selected from the group consisting of deuterium, a dibenzofuranyl, a carbazolyl, an isopropyl substituted with a phenyl(s), and a cumenyl, in which the substituents may be further substituted with deuterium.

According to one embodiment of the present disclosure, formula 1 is represented by at least one of the following formulas 1-1 to 1-28:

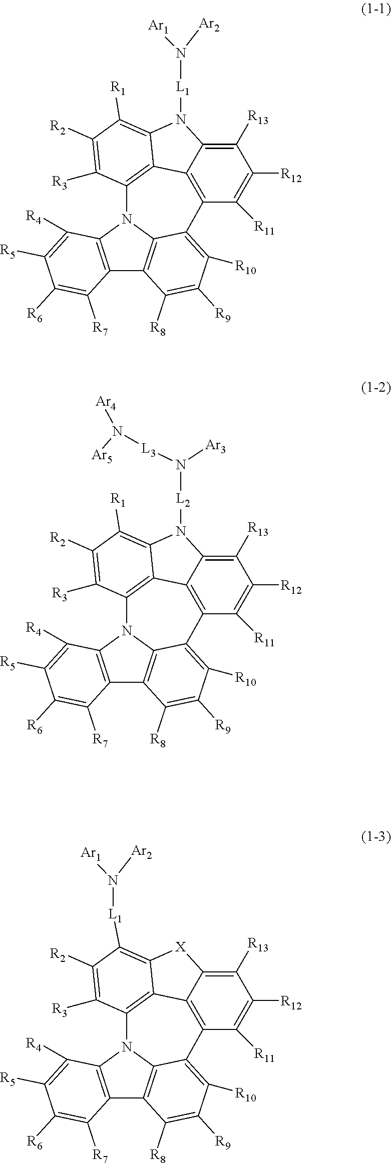

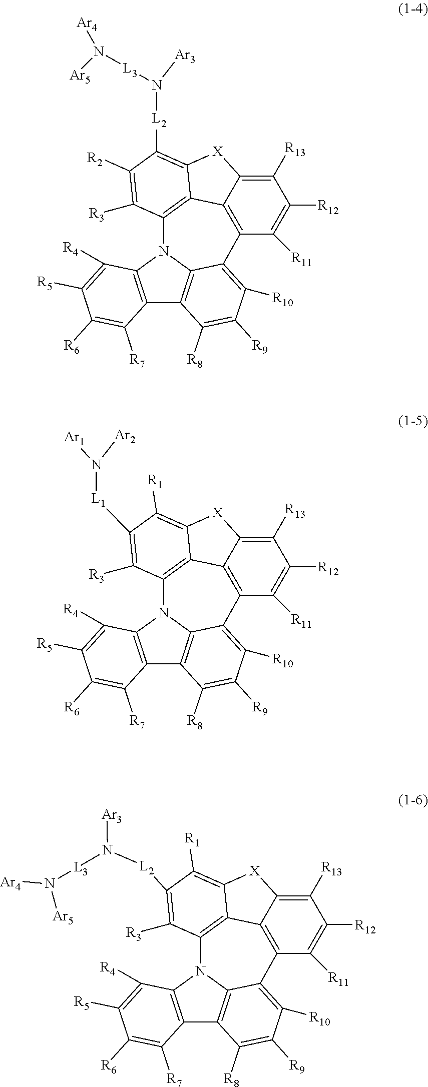

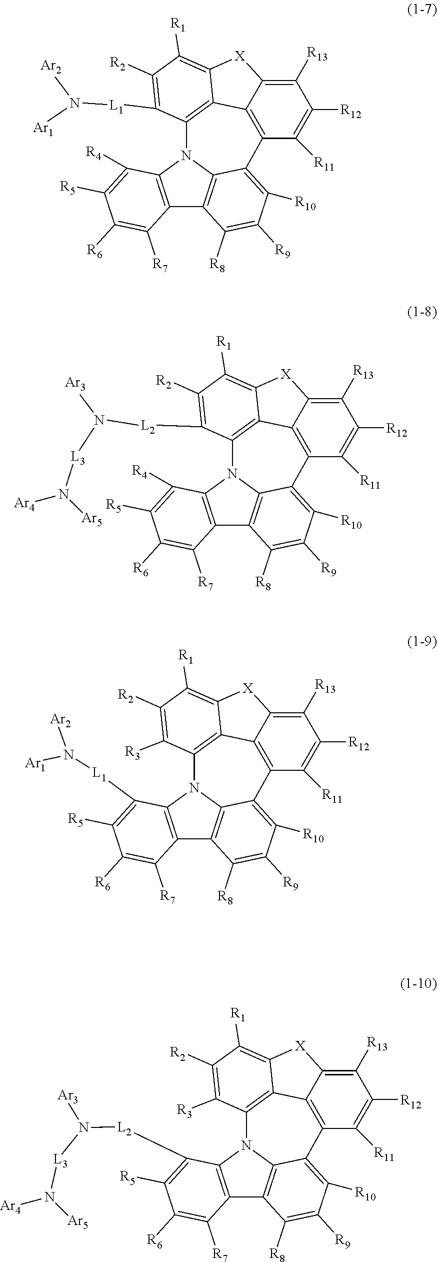

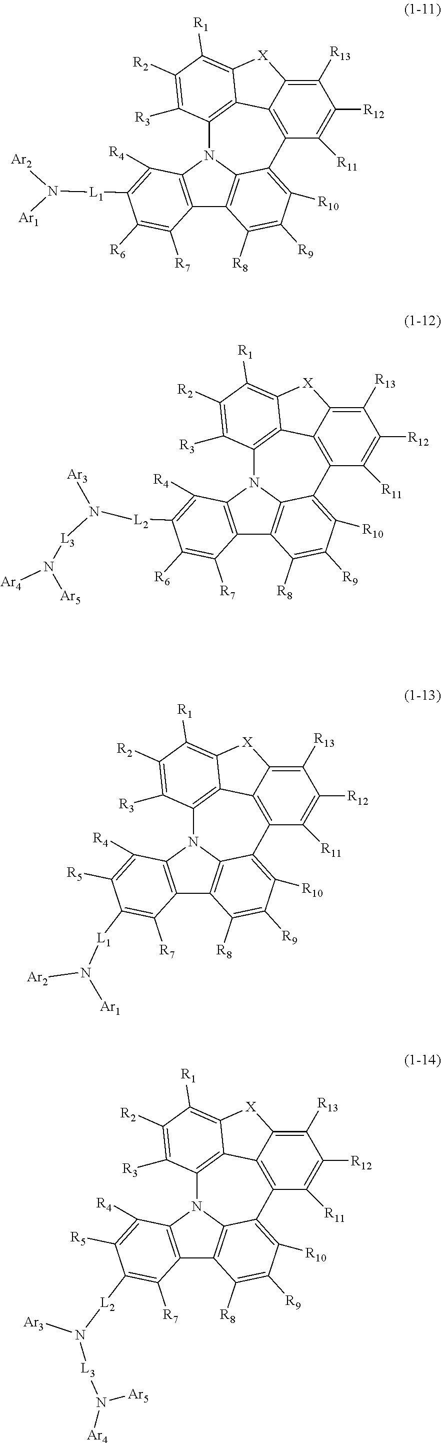

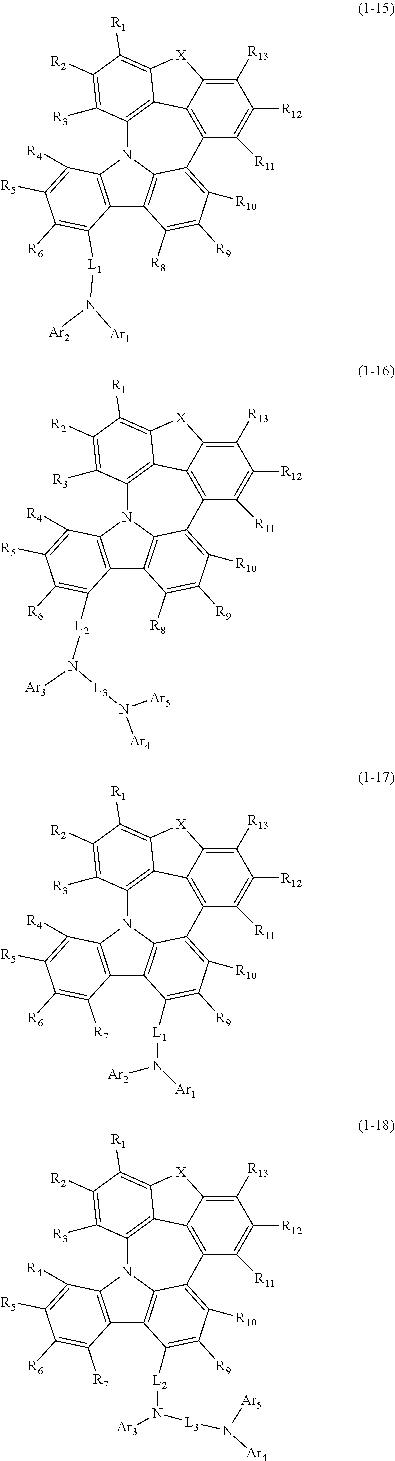

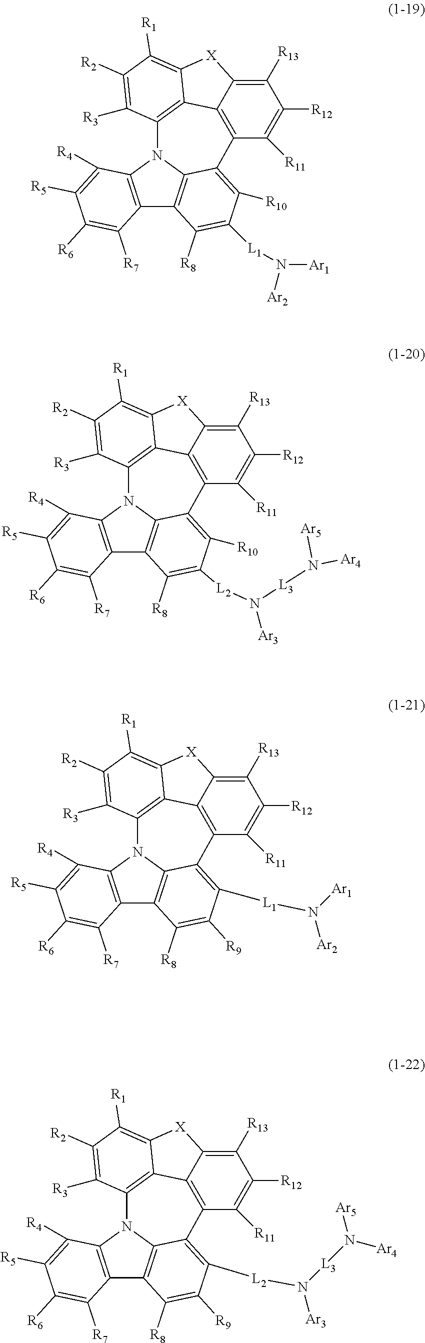

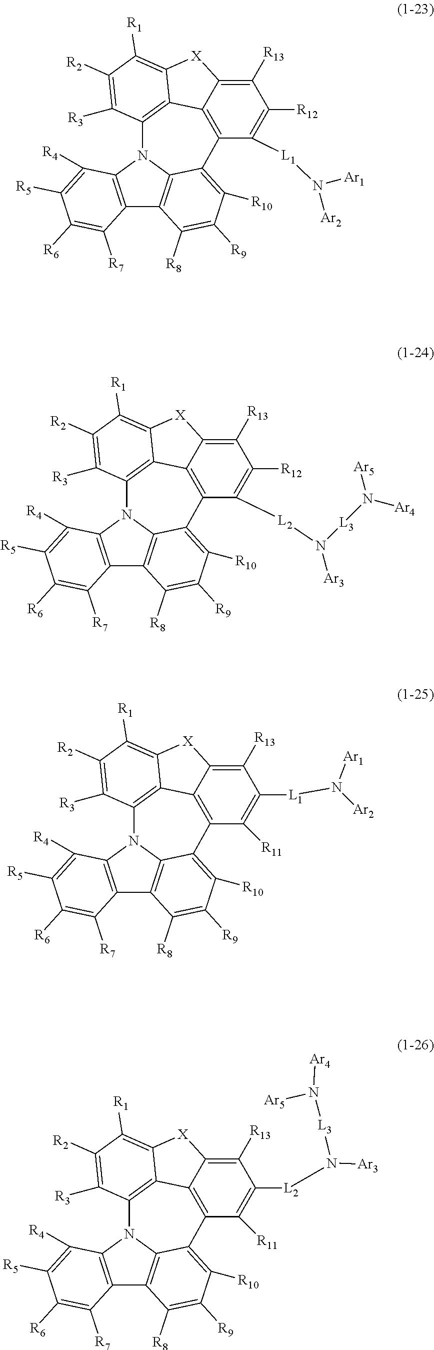

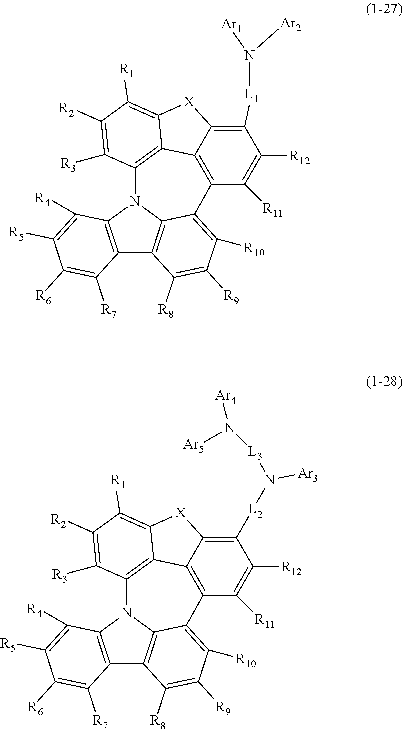

In formulas 1-1 to 1-28, the definitions and the preferred embodiments of X, R₁ to R₁₃, L₁ to L₃, and Ar₁ to Ar₅ are the same as in the formula 1.

The second host compound, which is another host material according to one embodiment, may be represented by formula 2.

In formula 2, X₁ to X₃, each independently, represent —N═ or —C(R₂₀)═, with the proviso that at least one of X₁ to X₃ represents N. According to one embodiment of the present disclosure, at least two of X₁ to X₃ are N. According to another embodiment of the present disclosure, all of X₁ to X₃ are N.

R₂₀ represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s). For example, R₂₀ may be hydrogen or deuterium.

In formula 2, L₄ to L₆, each independently, represent a single bond, a substituted or unsubstituted (C6-C30)arylene, a substituted or unsubstituted (C3-C30)cycloalkylene, or a substituted or unsubstituted (3- to 30-membered)heteroarylene. According to one embodiment of the present disclosure, L₄ to L₆, each independently, represent a single bond, a substituted or unsubstituted (C6-C25)arylene, or a substituted or unsubstituted (5- to 25-membered)heteroarylene. According to another embodiment of the present disclosure, L₄ to L₆, each independently, represent a single bond, a substituted or unsubstituted (C6-C18)arylene, or a substituted or unsubstituted (5- to 20-membered)heteroarylene. For example, L₄ to L₆, each independently, may be a single bond, a substituted or unsubstituted, phenylene, naphthylene, biphenylene, naphthylphenylene, phenylnaphthylene, fluorenylene, phenanthrenylene, pyridylene, carbazolylene, dibenzofuranylene, dibenzothiophenylene, benzonaphthothiophenylene, naphthooxazolylene, naphthothiazolylene, or benzonaphthofuranylene.

In formula 2, Ar₆ to Ar₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or *—N—(R₂₁)(R₂₂); or may be linked to an adjacent substituent(s) to form a ring(s); with the proviso that at least one of Ar₆ to Ar₈ represents a substituted or unsubstituted (C6-C30)aryl or a substituted or unsubstituted (3- to 30-membered)heteroaryl. According to one embodiment of the present disclosure, Ar₆ to Ar₅, each independently, represent a substituted or unsubstituted (C6-C25)aryl, a substituted or unsubstituted (5- to 25-membered)heteroaryl, or a substituted or unsubstituted tri(C6-C25)arylsilyl. According to another embodiment of the present disclosure, Ar₆ to Ar₈, each independently, represent a substituted or unsubstituted (C6-C25)aryl, a substituted or unsubstituted (5- to 18-membered)heteroaryl, or a substituted or unsubstituted tri(C6-C18)arylsilyl.

For example, at least one of Ar₆ to Ar₈ may be a substituted or unsubstituted (5- to 30-membered)heteroaryl, and preferably, at least two of Ar₆ to Ar₈ may be a substituted or unsubstituted (5- to 30-membered) heteroaryl.

For example, all of Ar₆ to Ar₈ may be a substituted or unsubstituted (C6-C30)aryl.

Specifically, Ar₆ to Ar₈, each independently, may be a substituted or unsubstituted, phenyl, naphthyl, p-biphenyl, m-biphenyl, o-biphenyl, o-terphenyl, m-terphenyl, p-terphenyl, triphenylsilyl, triphenylgermanyl, dibenzofuranyl, dibenzothiophenyl, benzoselenophenyl, fluorenyl, spirobifluorenyl, carbazolyl, phenanthrenyl, benzophenanthrenyl, chrysenyl, triphenylenyl, benzocarbazolyl, benzonaphthofuranyl, benzonaphthothiophenyl, benzofluorenyl, benzothiazolyl, benzoxazolyl, benzimidazolyl, naphthooxazolyl, benzonaphthooxazolyl, naphthothiazolyl, benzonaphthothiazolyl, or naphthoimidazolyl. For example, Ar₆ to Ar₈, each independently, may be a substituted or unsubstituted, phenyl, naphthyl, p-biphenyl, m-biphenyl, o-biphenyl, o-terphenyl, m-terphenyl, p-terphenyl, triphenylsilyl, triphenylgermanyl, dibenzofuranyl, dibenzothiophenyl, dibenzoselenophenyl, fluorenyl, benzofluorenyl, spirobifluorenyl, phenanthrenyl, chrysenyl, triphenylenyl, carbazolyl, benzonaphthofuranyl, benzonaphthothiophenyl, benzonaphthooxazolyl, benzonaphthothiazolyl, or naphthoselenazolyl. Herein, the substituent(s) of the above substituents may be at least one selected from the group consisting of deuterium, a cyano, a methyl, a phenyl, a biphenyl, a naphthyl, a phenanthrenyl, a triphenylsilyl, a fluorenyl, a dibenzothiophenyl, and a dibenzofuranyl.

According to one embodiment of the present disclosure, at least one of Ar₆ to Ar₈ may be any one selected from the following formulas 2-1 to 2-17.

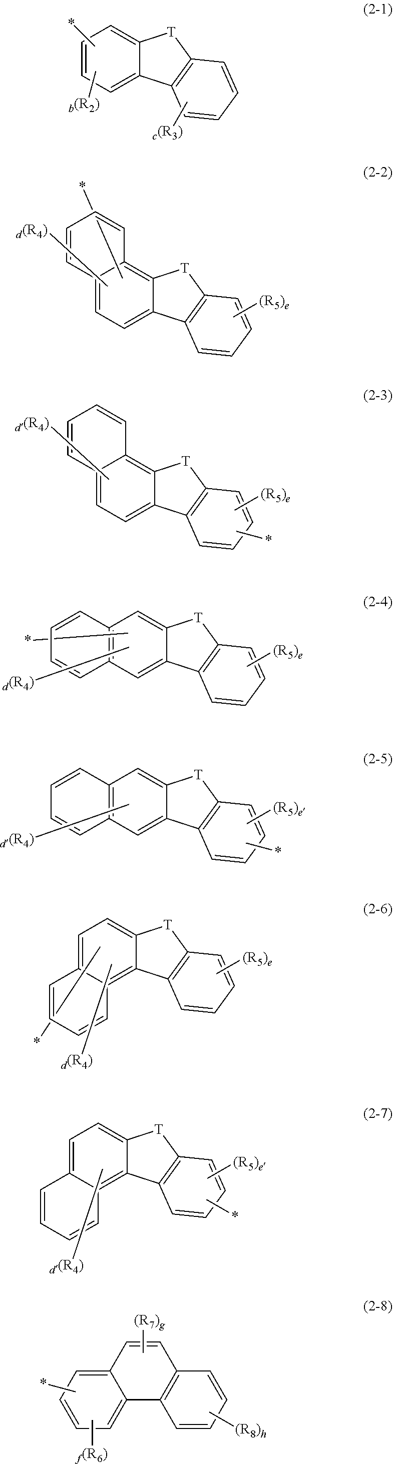

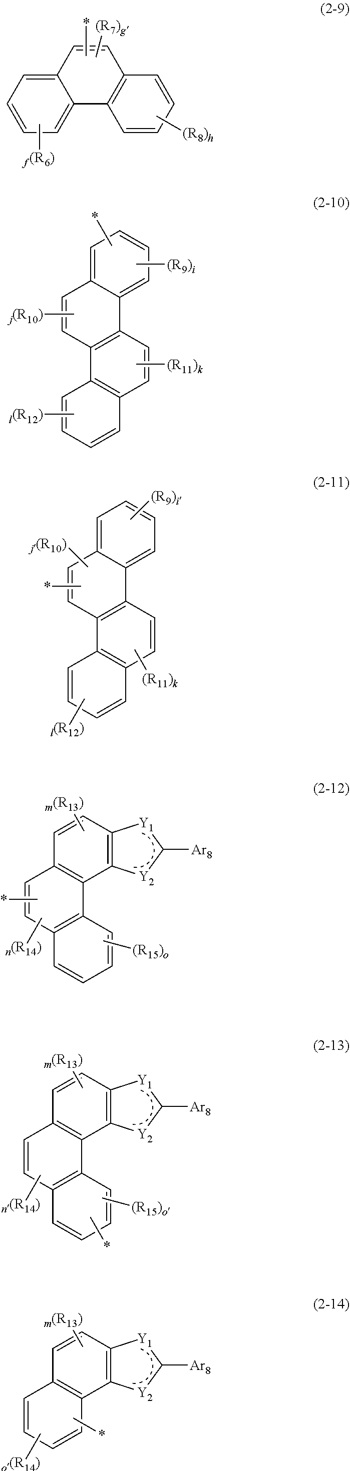

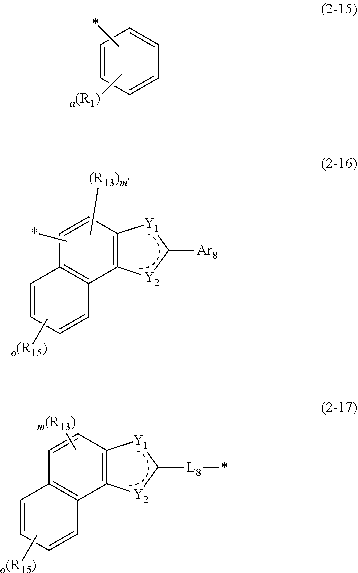

In formulas 2-1 to 2-17,
T represents O, S, C(R₁₇)(R₁₈), N(R₁₉), or Se;
Y₁ and Y₂, each independently, represent —N═, —N(R₂₁)—, —O—, —S—, or —Se—; provided that one of Y₁ and Y₂ represents —N═, the other of Y₁ and Y₂ represents —N(R₂₁)—, —O—, —S—, or —Se—;
R₁ to R₁₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or —N—(R₂₂)(R₂₃); or may be linked to an adjacent substituent(s) to form a ring(s);
R₂₂ and R₂₃, each independently, represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl;
R₁₇ to R₁₉ and R₂₁, each independently, represent a substituted or unsubstituted (C1-C30)alkyl, a substituted or unsubstituted (C6-C30)aryl, or a substituted or unsubstituted (3- to 30-membered)heteroaryl; or may be linked to an adjacent substituent(s) to form a ring(s);
Lₐ represents a single bond, a substituted or unsubstituted (C6-C30)arylene, or a substituted or unsubstituted (3- to 30-membered)heteroarylene;
Ar₈ represents a substituted or unsubstituted (C6-C30)aryl, or a substituted or unsubstituted (3- to 30-membered)heteroaryl;
a and d represent an integer of 1 to 5, b, e′, f, i, and o′ represent an integer of 1 to 3, c, e, h, f′, l, i′, and o represent an integer of 1 to 4, d′ represents an integer of 1 to 6, g, j, k, m, and n′ represent an integer of 1 or 2, and g′, j′, m′, and n represent an integer of 1;
if a to m, o, d′ to f, i′, n′, and o′ represent an integer of 2 or more, each of R₁ to each of R₁₅ may be the same or different; and
* is a linking position with L₄ to L₆ in formula 2.

For example, T may be O, S, Se, or CR₁₇R₁₈, wherein R₁₇ and R₁₃, each independently, may be a substituted or unsubstituted (C1-C30)alkyl, or a substituted or unsubstituted (C6-C30)aryl.

For example, one of Y₁ and Y₂ may be —N═, and the other of Y₁ and Y₂ may be —O—, —S—, or —Se—.

For example, R₁ to R₁₅, each independently, represent hydrogen, deuterium, a cyano, a substituted or unsubstituted (C6-C30)aryl, a substituted or unsubstituted (5- to 30-membered)heteroaryl, or a substituted or unsubstituted tri(C6-C30)arylsilyl, preferably may be hydrogen, deuterium, a substituted or unsubstituted (C6-C25)aryl, a substituted or unsubstituted (5- to 25-membered)heteroaryl, or a substituted or unsubstituted tri(C6-C25)arylsilyl, more preferably may be hydrogen, deuterium, a substituted or unsubstituted (C6-C18)aryl, a substituted or unsubstituted (5- to 18-membered)heteroaryl, or a substituted or unsubstituted tri(C6-C18)arylsilyl. For example, R₁ to R₁₅, each independently, represent hydrogen, deuterium, a cyano, a substituted or unsubstituted phenyl, a substituted or unsubstituted biphenyl, a substituted or unsubstituted naphthylphenyl, a substituted or unsubstituted naphthyl, a substituted or unsubstituted phenylnaphthyl, a substituted or unsubstituted phenanthrenyl, a substituted or unsubstituted fluorenyl, a substituted or unsubstituted triphenylsilyl, a substituted or unsubstituted dibenzofuranyl, a substituted or unsubstituted dibenzothiophenyl, or a substituted or unsubstituted benzonaphthooxazolyl.

For example, Ar₈ may be a substituted or unsubstituted (C6-C30)aryl, or a substituted or unsubstituted (5- to 30-membered)heteroaryl, preferably may be a substituted or unsubstituted (C6-C25)aryl, or a substituted or unsubstituted (5- to 25-membered)heteroaryl, more preferably may be a substituted or unsubstituted (C6-C18)aryl, or a substituted or unsubstituted (5- to 18-membered)heteroaryl. For example, Ar₈ may be a substituted or unsubstituted phenyl, a substituted or unsubstituted naphthyl, a substituted or unsubstituted biphenyl, a substituted or unsubstituted phenanthrenyl, a substituted or unsubstituted fluorenyl, a substituted or unsubstituted pyridyl, a substituted or unsubstituted dibenzofuranyl, or a substituted or unsubstituted dibenzothiophenyl. In this case, the substituent(s) of the substituents may be at least one selected from deuterium, a cyano, a methyl, a phenyl, a triphenylsilyl, and a triphenylgermanyl.

According to another embodiment of the present disclosure, the present disclosure provides an organic electroluminescent compound represented by formula 1′, and an organic electroluminescent device comprising the same.

In formula 1′, X represents —N(R)—, —C(R′)(R″)— or —Se—. In formula 1′, the definitions and preferred embodiments of R, R′, R″, and R₁ to R₁₃ are the same as in formula 1.

According to another embodiment of the present disclosure, the present disclosure provides an organic electroluminescent compound represented by any one of formulas 2′-1 to 2′-4, and an organic electroluminescent device comprising the same. The compound represented by formula 1 or 1′ may be at least one selected from the group consisting of the following compounds, but is not limited thereto.

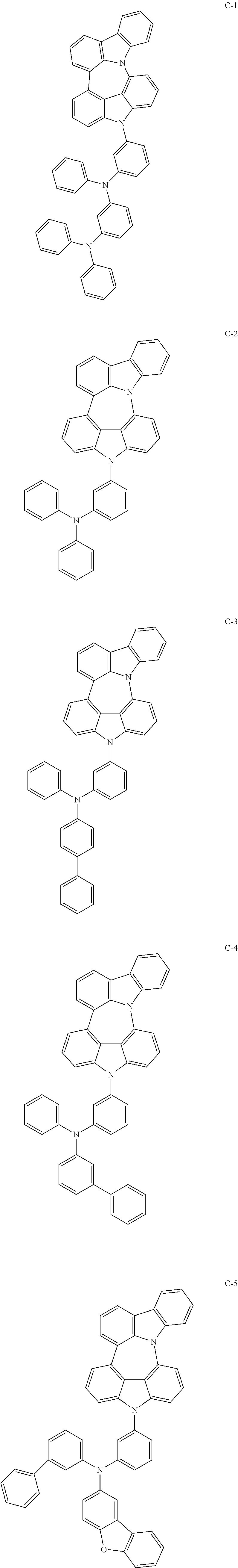 [SMILES: c1ccc(N(c2ccccc2)c2cccc(N(c3ccccc3)c3cccc(-n4c5cccc6c7cccc8c9ccccc9n(c9cccc4c9c65)c78)c3)c2)cc1 ; c1ccc(N(c2ccccc2)c2cccc(-n3c4cccc5c6cccc7c8ccccc8n(c8cccc3c8c54)c67)c2)cc1 ; c1ccc(-c2ccc(N(c3ccccc3)c3cccc(-n4c5cccc6c7cccc8c9ccccc9n(c9cccc4c9c65)c78)c3)cc2)cc1 ; c1ccc(-c2cccc(N(c3ccccc3)c3cccc(-n4c5cccc6c7cccc8c9ccccc9n(c9cccc4c9c65)c78)c3)c2)cc1 ; c1ccc(-c2cccc(N(c3cccc(-n4c5cccc6c7cccc8c9ccccc9n(c9cccc4c9c65)c78)c3)c3ccc4oc5ccccc5c4c3)c2)cc1]

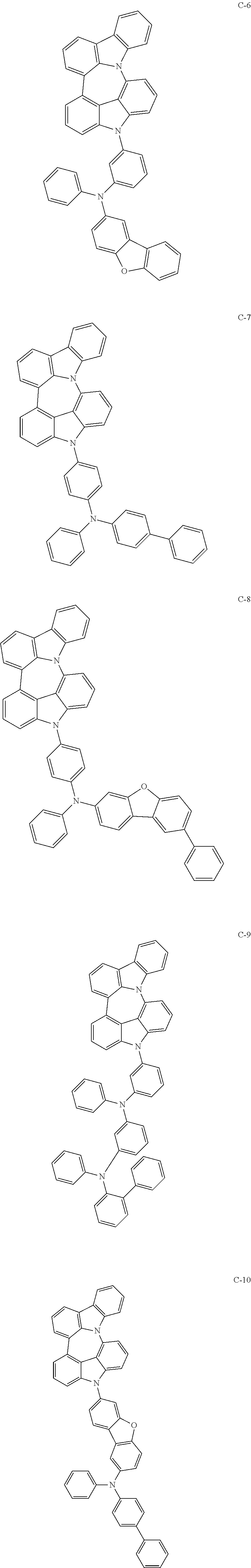

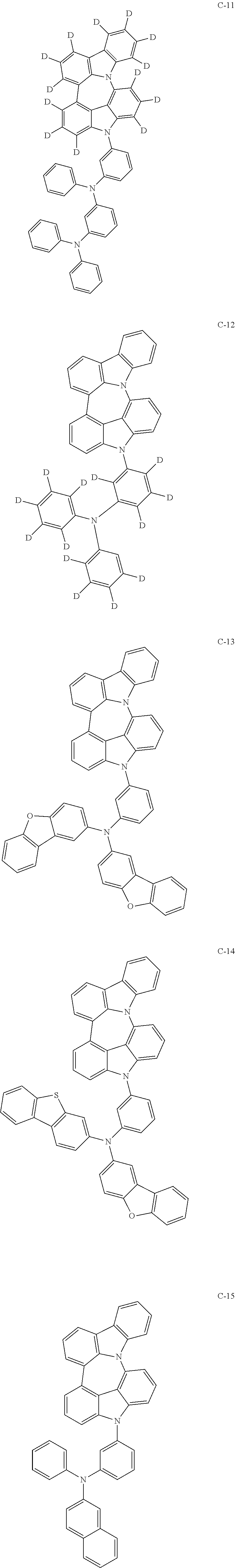

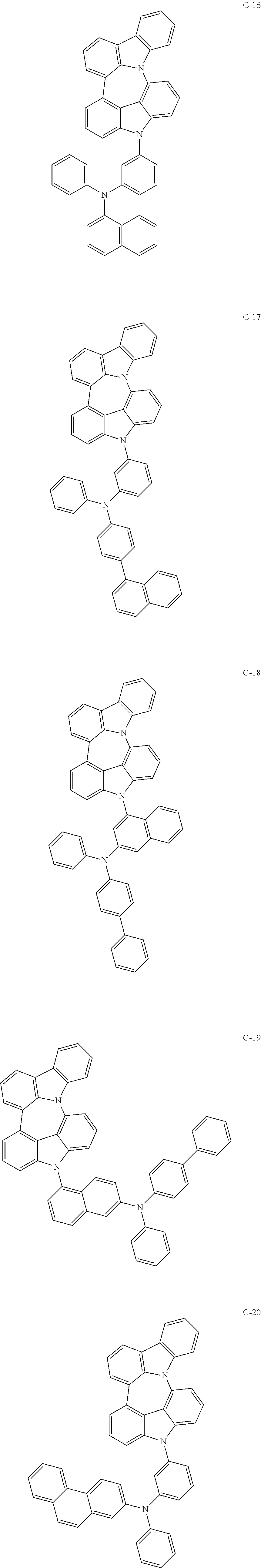 [SMILES: c1ccc(N(c2cccc(-n3c4cccc5c6cccc7c8ccccc8n(c8cccc3c8c54)c67)c2)c2cccc3ccccc23)cc1 ; c1ccc(N(c2ccc(-c3cccc4ccccc34)cc2)c2cccc(-n3c4cccc5c6cccc7c8ccccc8n(c8cccc3c8c54)c67)c2)cc1 ; c1ccc(-c2ccc(N(c3ccccc3)c3cc(-n4c5cccc6c7cccc8c9ccccc9n(c9cccc4c9c65)c78)c4ccccc4c3)cc2)cc1 ; c1ccc(-c2ccc(N(c3ccccc3)c3ccc4c(-n5c6cccc7c8cccc9c%10ccccc%10n(c%10cccc5c%10c76)c89)cccc4c3)cc2)cc1 ; c1ccc(N(c2cccc(-n3c4cccc5c6cccc7c8ccccc8n(c8cccc3c8c54)c67)c2)c2ccc3c(ccc4ccccc43)c2)cc1]

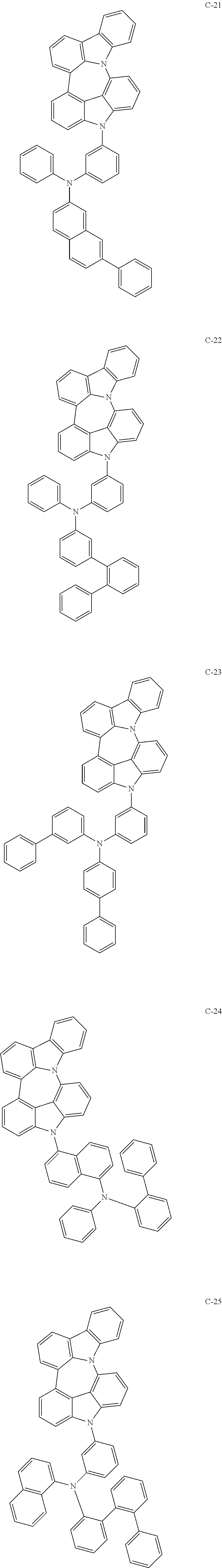 [SMILES: c1ccc(-c2ccc3ccc(N(c4ccccc4)c4cccc(-n5c6cccc7c8cccc9c%10ccccc%10n(c%10cccc5c%10c76)c89)c4)cc3c2)cc1 ; c1ccc(-c2ccccc2-c2cccc(N(c3ccccc3)c3cccc(-n4c5cccc6c7cccc8c9ccccc9n(c9cccc4c9c65)c78)c3)c2)cc1 ; c1ccc(-c2ccc(N(c3cccc(-c4ccccc4)c3)c3cccc(-n4c5cccc6c7cccc8c9ccccc9n(c9cccc4c9c65)c78)c3)cc2)cc1 ; c1ccc(-c2ccccc2N(c2ccccc2)c2cccc3c(-n4c5cccc6c7cccc8c9ccccc9n(c9cccc4c9c65)c78)cccc23)cc1 ; c1ccc(-c2ccccc2-c2ccccc2N(c2cccc(-n3c4cccc5c6cccc7c8ccccc8n(c8cccc3c8c54)c67)c2)c2cccc3ccccc23)cc1]

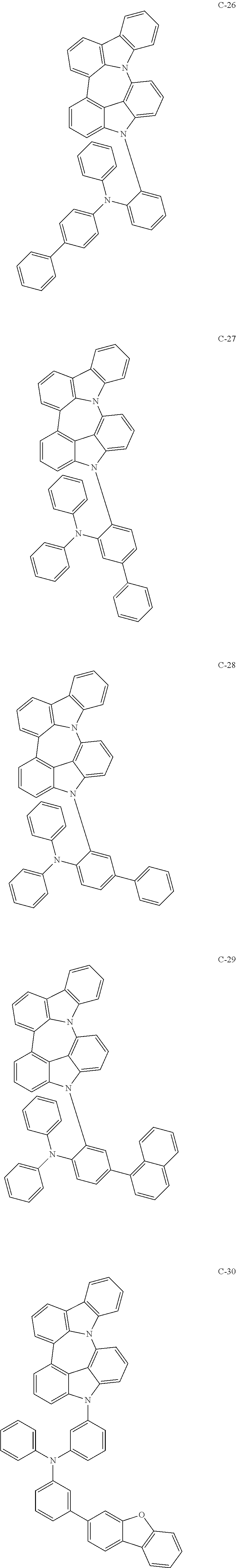 [SMILES: c1ccc(-c2ccc(N(c3ccccc3)c3ccccc3-n3c4cccc5c6cccc7c8ccccc8n(c8cccc3c8c54)c67)cc2)cc1 ; c1ccc(-c2ccc(-n3c4cccc5c6cccc7c8ccccc8n(c8cccc3c8c54)c67)c(N(c3ccccc3)c3ccccc3)c2)cc1 ; c1ccc(-c2ccc(N(c3ccccc3)c3ccccc3)c(-n3c4cccc5c6cccc7c8ccccc8n(c8cccc3c8c54)c67)c2)cc1 ; c1ccc(N(c2ccccc2)c2ccc(-c3cccc4ccccc34)cc2-n2c3cccc4c5cccc6c7ccccc7n(c7cccc2c7c43)c56)cc1 ; c1ccc(N(c2cccc(-c3ccc4c(c3)oc3ccccc34)c2)c2cccc(-n3c4cccc5c6cccc7c8ccccc8n(c8cccc3c8c54)c67)c2)cc1]

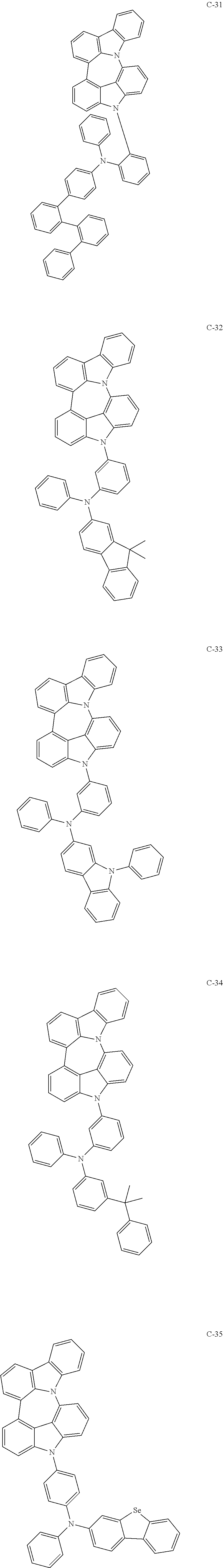 [SMILES: c1ccc(-c2ccccc2-c2ccccc2-c2ccc(N(c3ccccc3)c3ccccc3-n3c4cccc5c6cccc7c8ccccc8n(c8cccc3c8c54)c67)cc2)cc1 ; CC1(C)c2ccccc2-c2ccc(N(c3ccccc3)c3cccc(-n4c5cccc6c7cccc8c9ccccc9n(c9cccc4c9c65)c78)c3)cc21 ; c1ccc(N(c2cccc(-n3c4cccc5c6cccc7c8ccccc8n(c8cccc3c8c54)c67)c2)c2ccc3c4ccccc4n(-c4ccccc4)c3c2)cc1 ; CC(C)(c1ccccc1)c1cccc(N(c2ccccc2)c2cccc(-n3c4cccc5c6cccc7c8ccccc8n(c8cccc3c8c54)c67)c2)c1 ; c1ccc(N(c2ccc(-n3c4cccc5c6cccc7c8ccccc8n(c8cccc3c8c54)c67)cc2)c2ccc3c(c2)[se]c2ccccc23)cc1]

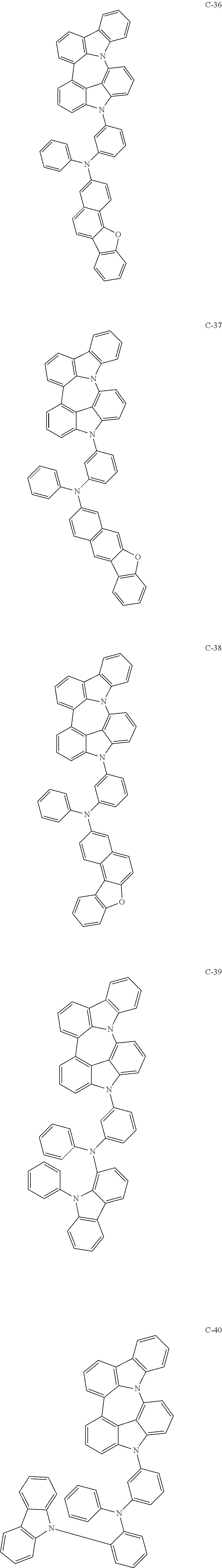 [SMILES: c1ccc(N(c2cccc(-n3c4cccc5c6cccc7c8ccccc8n(c8cccc3c8c54)c67)c2)c2ccc3c(ccc4c5ccccc5oc34)c2)cc1 ; c1ccc(N(c2cccc(-n3c4cccc5c6cccc7c8ccccc8n(c8cccc3c8c54)c67)c2)c2ccc3cc4c(cc3c2)oc2ccccc24)cc1 ; c1ccc(N(c2cccc(-n3c4cccc5c6cccc7c8ccccc8n(c8cccc3c8c54)c67)c2)c2ccc3c(ccc4oc5ccccc5c43)c2)cc1 ; c1ccc(N(c2cccc(-n3c4cccc5c6cccc7c8ccccc8n(c8cccc3c8c54)c67)c2)c2cccc3c4ccccc4n(-c4ccccc4)c23)cc1 ; c1ccc(N(c2cccc(-n3c4cccc5c6cccc7c8ccccc8n(c8cccc3c8c54)c67)c2)c2ccccc2-n2c3ccccc3c3ccccc32)cc1]

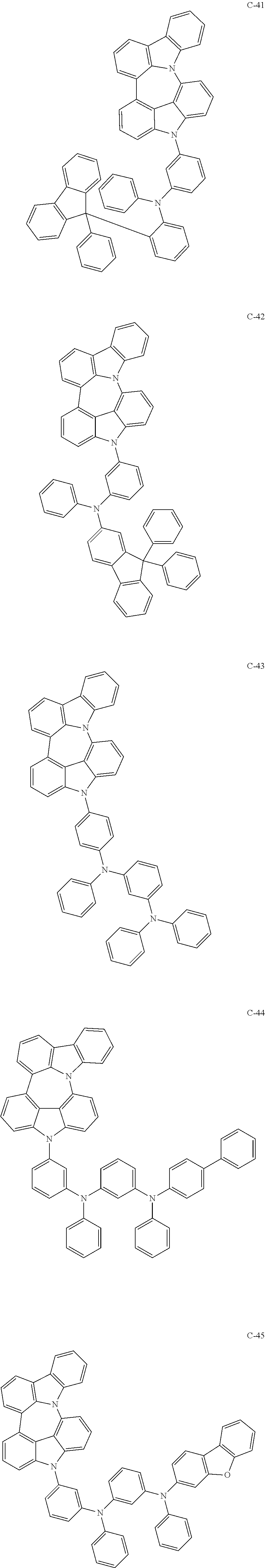

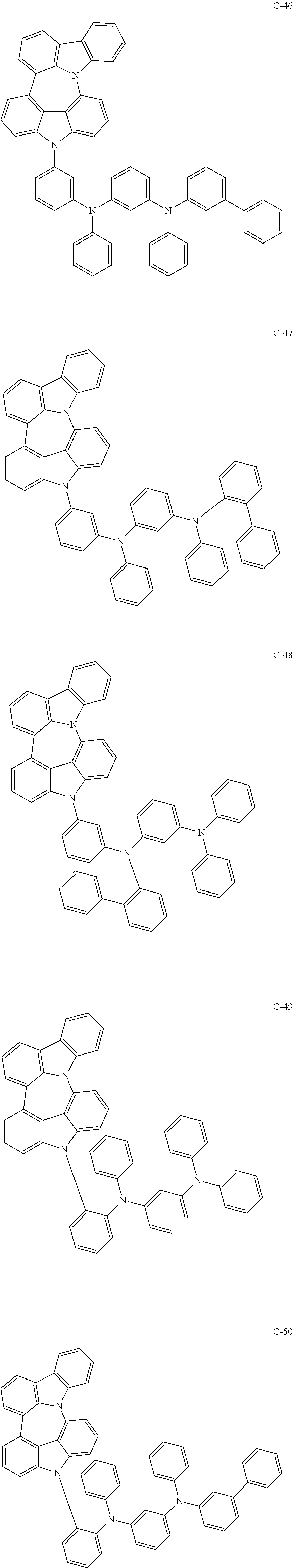

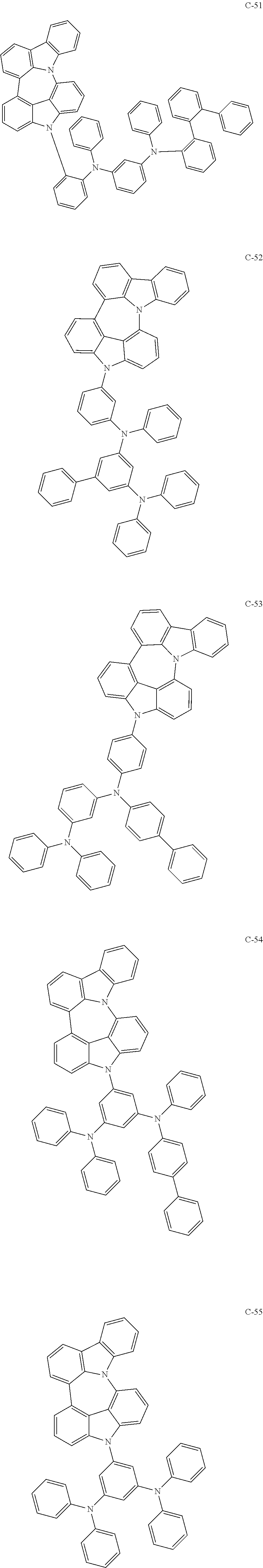

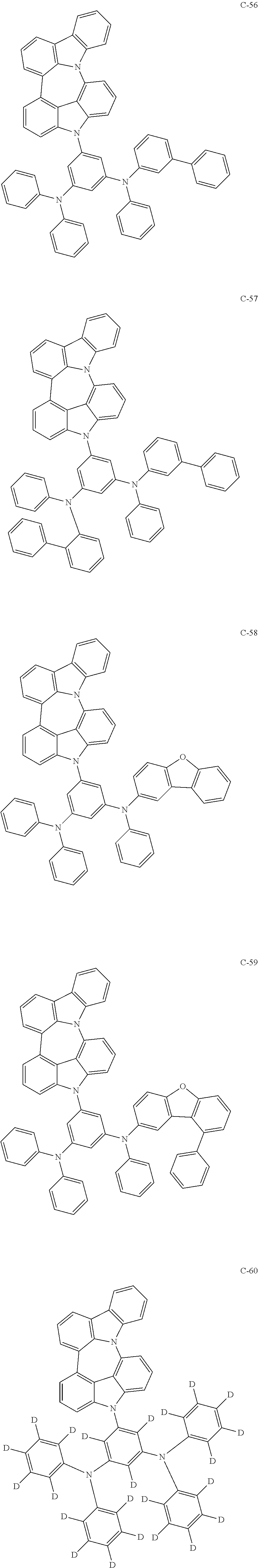

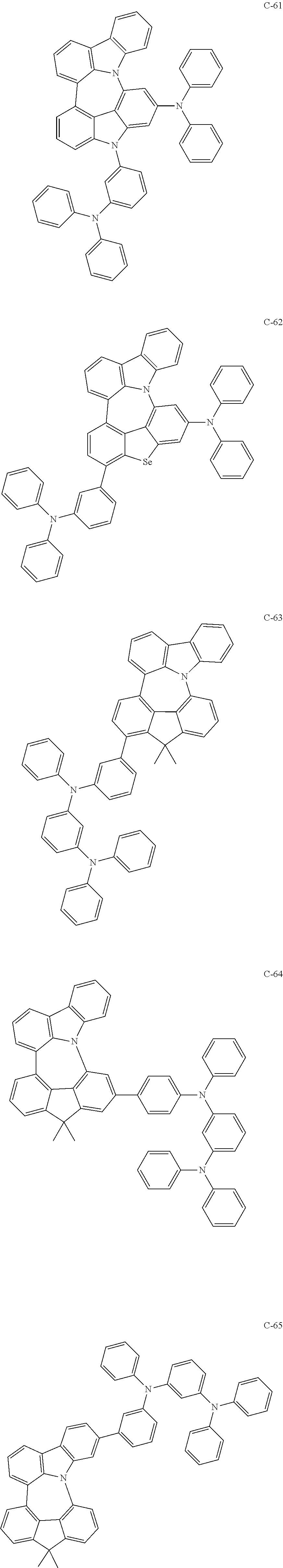

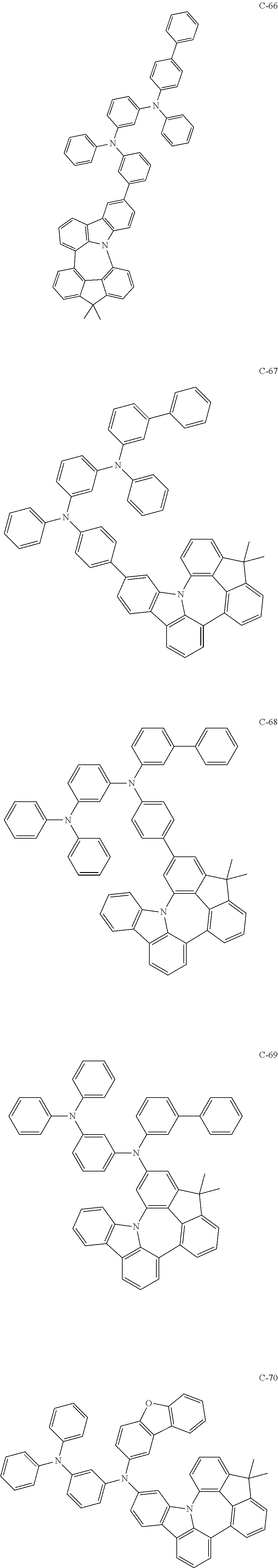

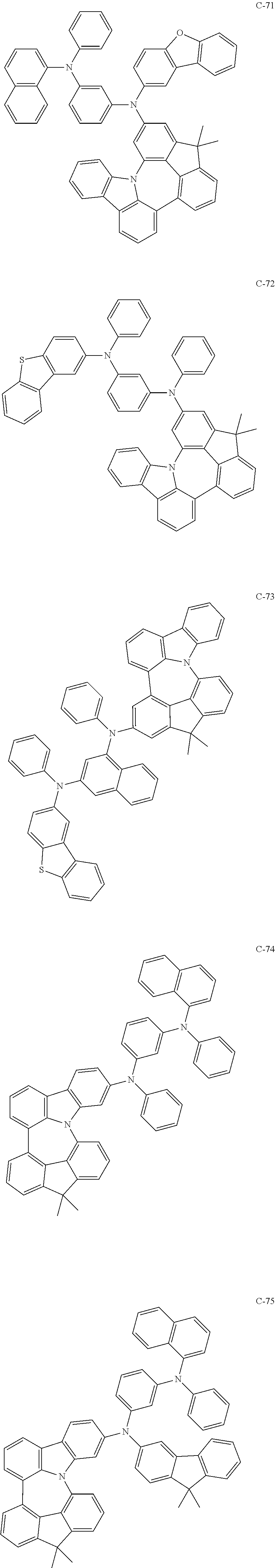

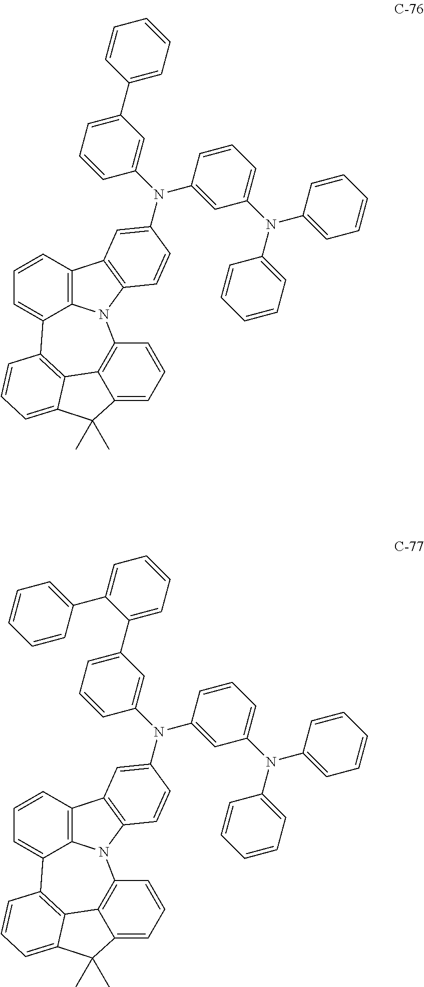 [SMILES: CC1(C)c2cccc3c2-c2c(cccc21)-n1c2ccc(N(c4cccc(-c5ccccc5)c4)c4cccc(N(c5ccccc5)c5ccccc5)c4)cc2c2cccc-3c21 ; CC1(C)c2cccc3c2-c2c(cccc21)-n1c2ccc(N(c4cccc(-c5ccccc5-c5ccccc5)c4)c4cccc(N(c5ccccc5)c5ccccc5)c4)cc2c2cccc-3c21]

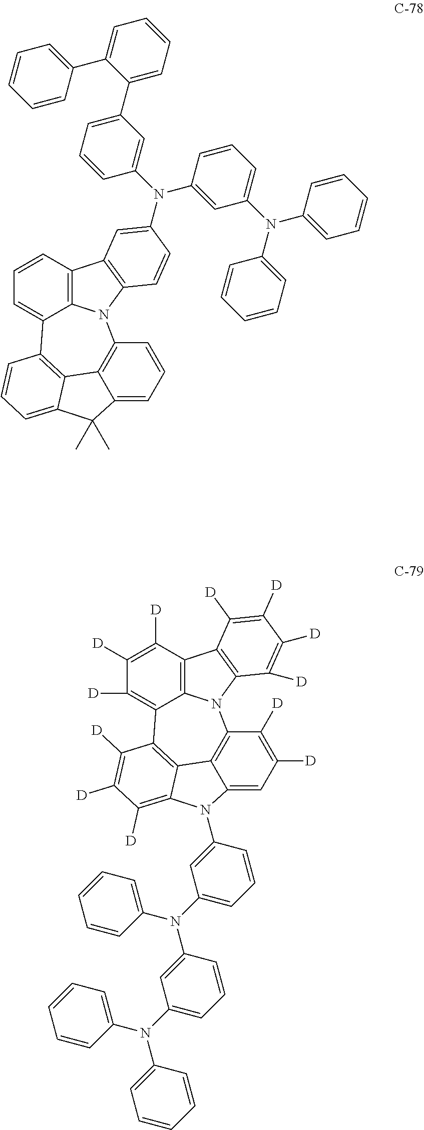 [SMILES: CC1(C)c2cccc3c2-c2c(cccc21)-n1c2ccc(N(c4cccc(-c5ccccc5-c5ccccc5)c4)c4cccc(N(c5ccccc5)c5ccccc5)c4)cc2c2cccc-3c21 ; [2H]c1cc2c3c4c(c([2H])c([2H])c([2H])c4n2-c2cccc(N(c4ccccc4)c4cccc(N(c5ccccc5)c5ccccc5)c4)c2)c2c([2H])c([2H])c([2H])c4c5c([2H])c([2H])c([2H])c([2H])c5n(c3c1[2H])c24]

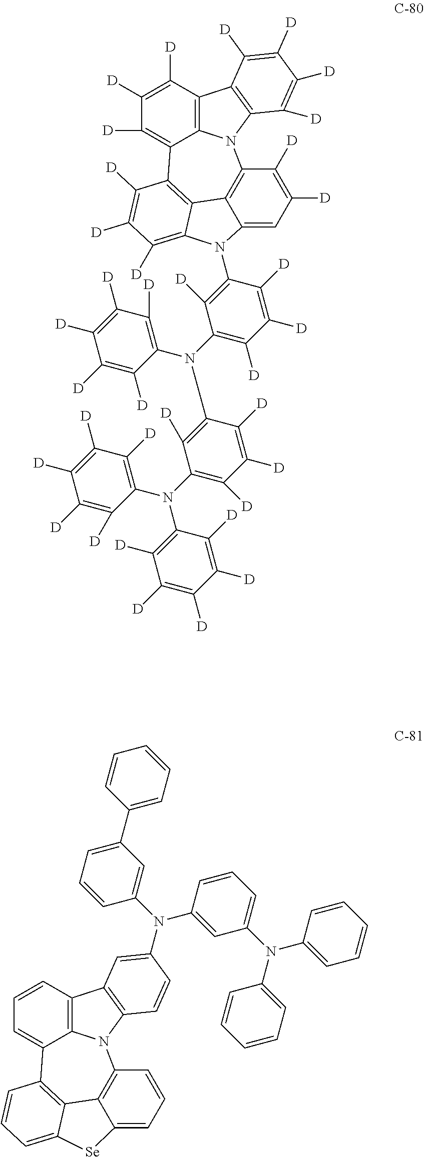 [SMILES: [2H]c1cc2c3c4c(c([2H])c([2H])c([2H])c4n2-c2c([2H])c([2H])c([2H])c(N(c4c([2H])c([2H])c([2H])c([2H])c4[2H])c4c([2H])c([2H])c([2H])c(N(c5c([2H])c([2H])c([2H])c([2H])c5[2H])c5c([2H])c([2H])c([2H])c([2H])c5[2H])c4[2H])c2[2H])c2c([2H])c([2H])c([2H])c4c5c([2H])c([2H])c([2H])c([2H])c5n(c3c1[2H])c24 ; c1ccc(-c2cccc(N(c3cccc(N(c4ccccc4)c4ccccc4)c3)c3ccc4c(c3)c3cccc5c6cccc7[se]c8cccc(c8c76)n4c53)c2)cc1]

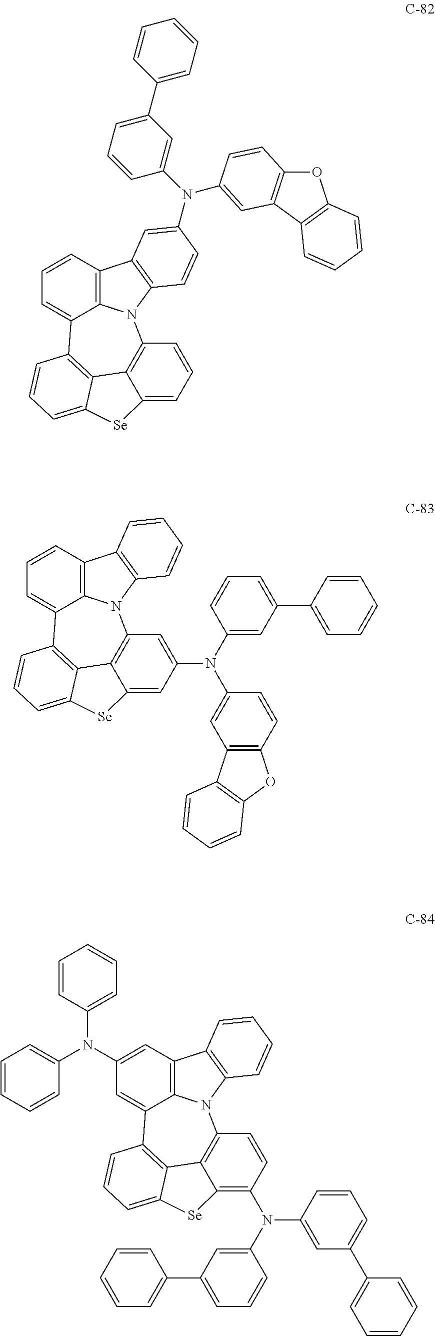 [SMILES: c1ccc(-c2cccc(N(c3ccc4oc5ccccc5c4c3)c3ccc4c(c3)c3cccc5c6cccc7[se]c8cccc(c8c76)n4c53)c2)cc1 ; c1ccc(-c2cccc(N(c3ccc4oc5ccccc5c4c3)c3cc4[se]c5cccc6c7cccc8c9ccccc9n(c(c3)c4c56)c78)c2)cc1 ; c1ccc(-c2cccc(N(c3cccc(-c4ccccc4)c3)c3ccc4c5c3[se]c3cccc(c6cc(N(c7ccccc7)c7ccccc7)cc7c8ccccc8n4c67)c35)c2)cc1]

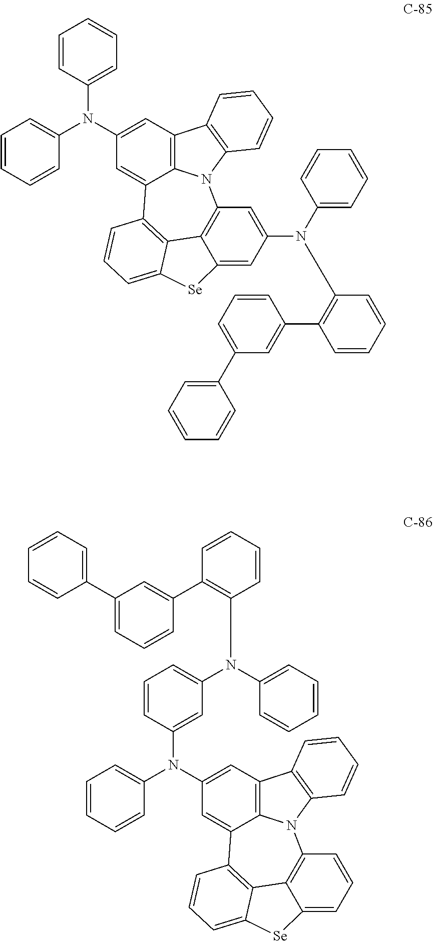 [SMILES: c1ccc(-c2cccc(-c3ccccc3N(c3ccccc3)c3cc4[se]c5cccc6c7cc(N(c8ccccc8)c8ccccc8)cc8c9ccccc9n(c(c3)c4c56)c78)c2)cc1 ; c1ccc(-c2cccc(-c3ccccc3N(c3ccccc3)c3cccc(N(c4ccccc4)c4cc5c6cccc7[se]c8cccc(c8c76)n6c7ccccc7c(c4)c56)c3)c2)cc1]

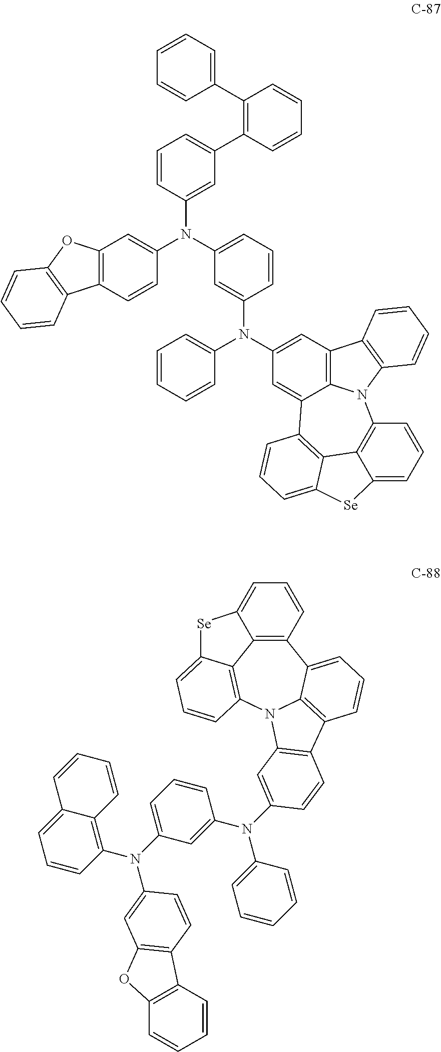 [SMILES: c1ccc(-c2ccccc2-c2cccc(N(c3cccc(N(c4ccccc4)c4cc5c6cccc7[se]c8cccc(c8c76)n6c7ccccc7c(c4)c56)c3)c3ccc4c(c3)oc3ccccc34)c2)cc1 ; c1ccc(N(c2cccc(N(c3ccc4c(c3)oc3ccccc34)c3cccc4ccccc34)c2)c2ccc3c4cccc5c6cccc7[se]c8cccc(c8c76)n(c3c2)c54)cc1]

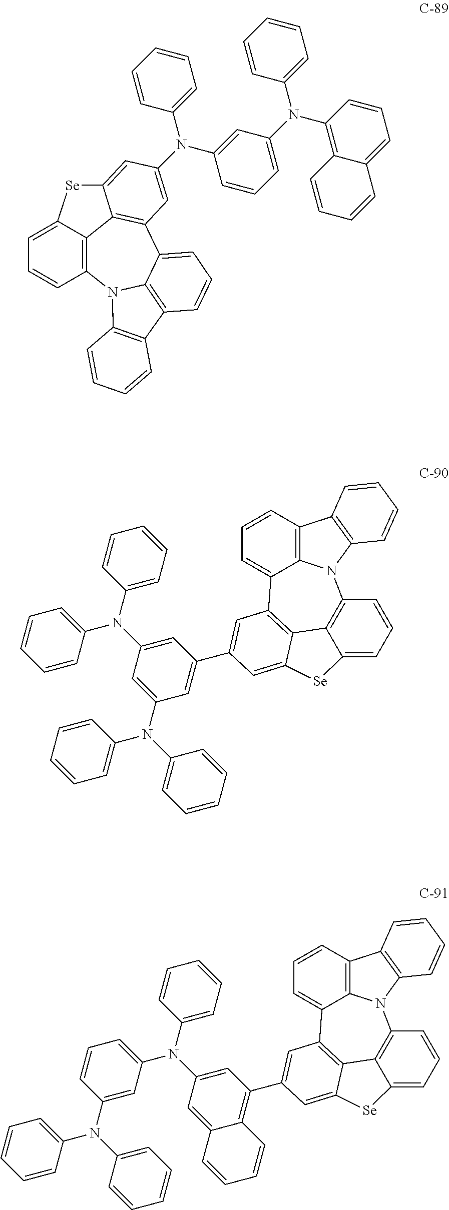 [SMILES: c1ccc(N(c2cccc(N(c3ccccc3)c3cccc4ccccc34)c2)c2cc3[se]c4cccc5c4c3c(c2)c2cccc3c4ccccc4n5c23)cc1 ; c1ccc(N(c2ccccc2)c2cc(-c3cc4[se]c5cccc6c5c4c(c3)c3cccc4c5ccccc5n6c34)cc(N(c3ccccc3)c3ccccc3)c2)cc1 ; c1ccc(N(c2ccccc2)c2cccc(N(c3ccccc3)c3cc(-c4cc5[se]c6cccc7c6c5c(c4)c4cccc5c6ccccc6n7c45)c4ccccc4c3)c2)cc1]

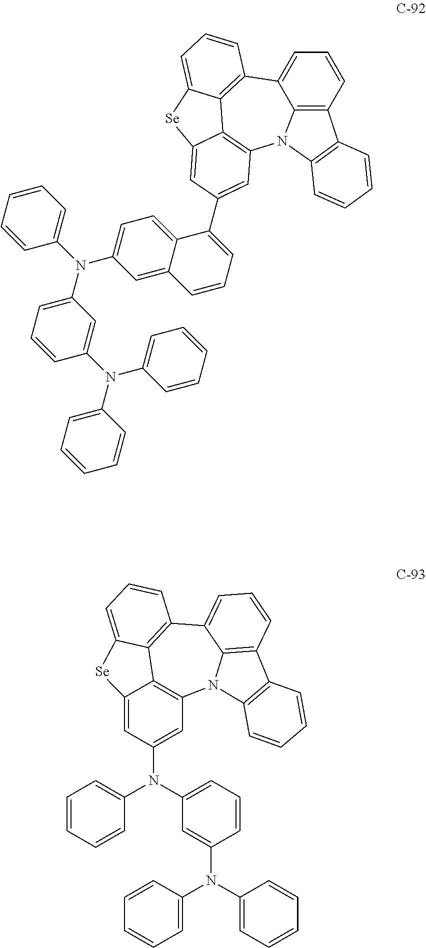 [SMILES: c1ccc(N(c2ccccc2)c2cccc(N(c3ccccc3)c3ccc4c(-c5cc6[se]c7cccc8c9cccc%10c%11ccccc%11n(c(c5)c6c78)c9%10)cccc4c3)c2)cc1 ; c1ccc(N(c2ccccc2)c2cccc(N(c3ccccc3)c3cc4[se]c5cccc6c7cccc8c9ccccc9n(c(c3)c4c56)c78)c2)cc1]

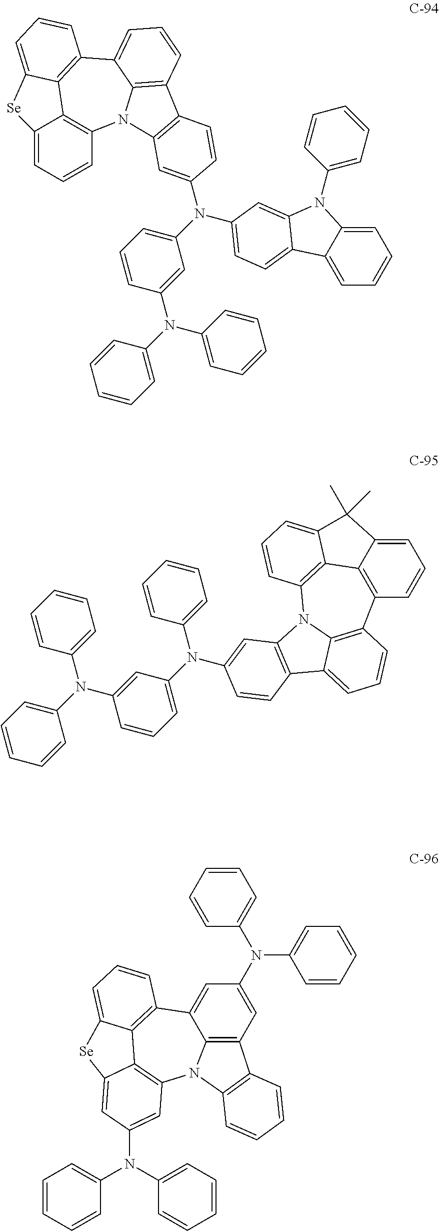 [SMILES: c1ccc(N(c2ccccc2)c2cccc(N(c3ccc4c5ccccc5n(-c5ccccc5)c4c3)c3ccc4c5cccc6c7cccc8[se]c9cccc(c9c87)n(c4c3)c65)c2)cc1 ; CC1(C)c2cccc3c2-c2c(cccc21)-n1c2cc(N(c4ccccc4)c4cccc(N(c5ccccc5)c5ccccc5)c4)ccc2c2cccc-3c21 ; c1ccc(N(c2ccccc2)c2cc3c4cccc5[se]c6cc(N(c7ccccc7)c7ccccc7)cc(c6c54)n4c5ccccc5c(c2)c34)cc1]

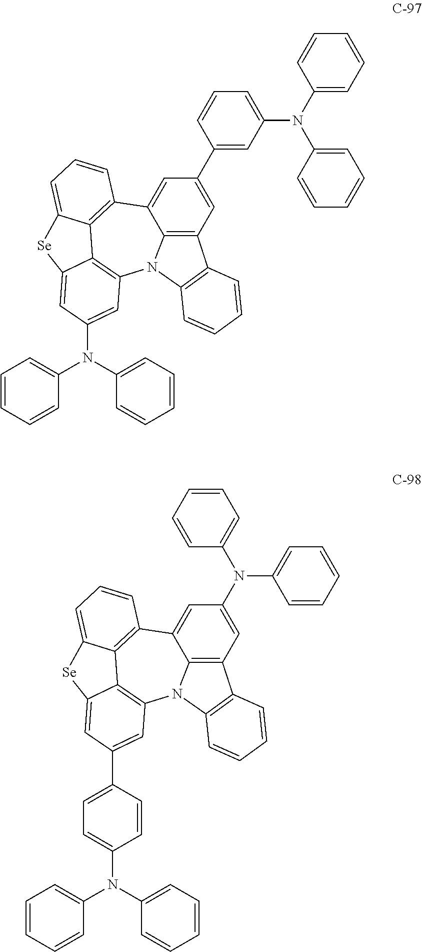 [SMILES: c1ccc(N(c2ccccc2)c2cccc(-c3cc4c5cccc6[se]c7cc(N(c8ccccc8)c8ccccc8)cc(c7c65)n5c6ccccc6c(c3)c45)c2)cc1 ; c1ccc(N(c2ccccc2)c2ccc(-c3cc4[se]c5cccc6c7cc(N(c8ccccc8)c8ccccc8)cc8c9ccccc9n(c(c3)c4c56)c78)cc2)cc1]

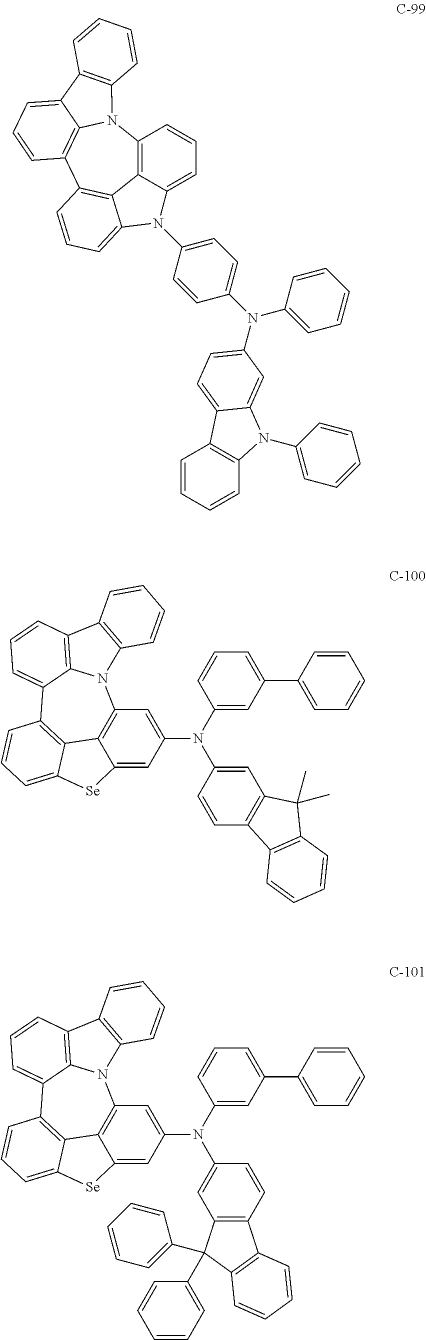 [SMILES: c1ccc(N(c2ccc(-n3c4cccc5c6cccc7c8ccccc8n(c8cccc3c8c54)c67)cc2)c2ccc3c4ccccc4n(-c4ccccc4)c3c2)cc1 ; CC1(C)c2ccccc2-c2ccc(N(c3cccc(-c4ccccc4)c3)c3cc4[se]c5cccc6c7cccc8c9ccccc9n(c(c3)c4c56)c78)cc21 ; c1ccc(-c2cccc(N(c3ccc4c(c3)C(c3ccccc3)(c3ccccc3)c3ccccc3-4)c3cc4[se]c5cccc6c7cccc8c9ccccc9n(c(c3)c4c56)c78)c2)cc1]

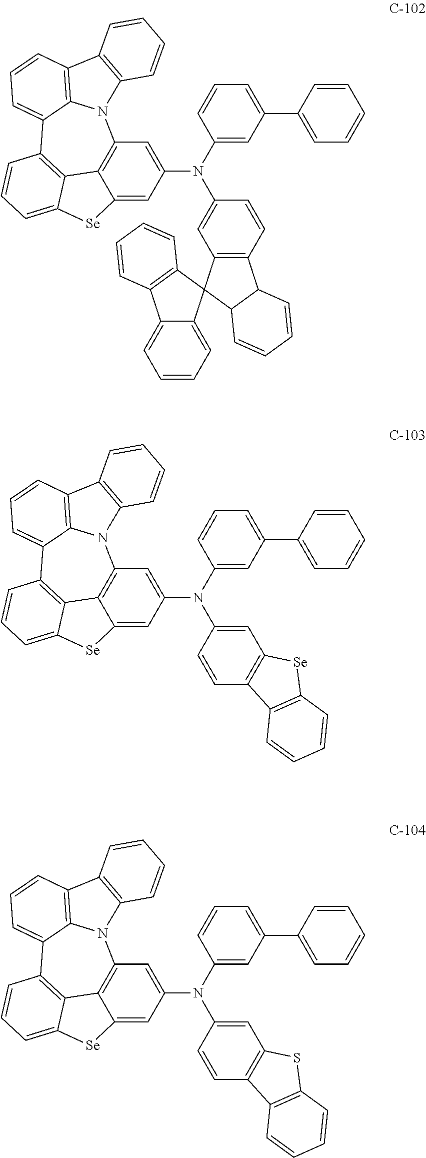 [SMILES: C1=CC2c3ccc(N(c4cccc(-c5ccccc5)c4)c4cc5[se]c6cccc7c8cccc9c%10ccccc%10n(c(c4)c5c67)c89)cc3C3(c4ccccc4-c4ccccc43)C2C=C1 ; c1ccc(-c2cccc(N(c3ccc4c(c3)[se]c3ccccc34)c3cc4[se]c5cccc6c7cccc8c9ccccc9n(c(c3)c4c56)c78)c2)cc1 ; c1ccc(-c2cccc(N(c3ccc4c(c3)sc3ccccc34)c3cc4[se]c5cccc6c7cccc8c9ccccc9n(c(c3)c4c56)c78)c2)cc1]

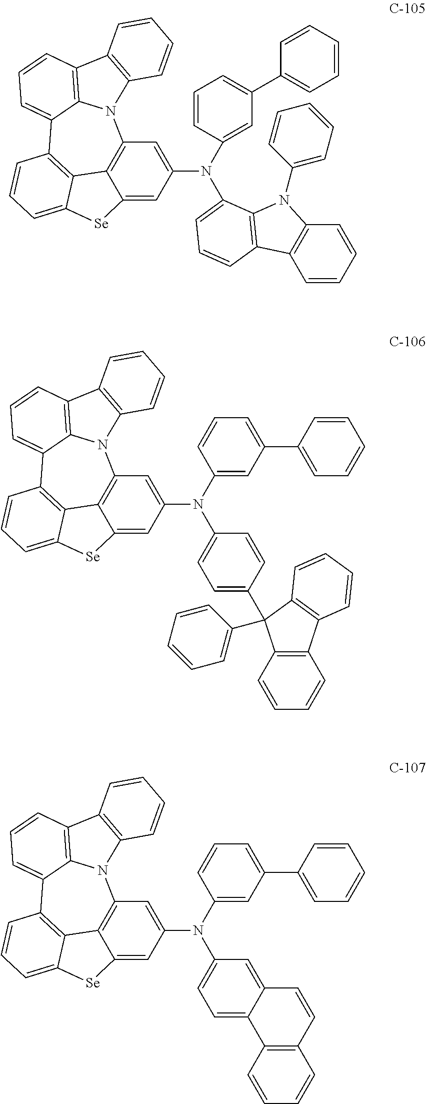 [SMILES: c1ccc(-c2cccc(N(c3cc4[se]c5cccc6c7cccc8c9ccccc9n(c(c3)c4c56)c78)c3cccc4c5ccccc5n(-c5ccccc5)c34)c2)cc1 ; c1ccc(-c2cccc(N(c3ccc(C4(c5ccccc5)c5ccccc5-c5ccccc54)cc3)c3cc4[se]c5cccc6c7cccc8c9ccccc9n(c(c3)c4c56)c78)c2)cc1 ; c1ccc(-c2cccc(N(c3ccc4c(ccc5ccccc54)c3)c3cc4[se]c5cccc6c7cccc8c9ccccc9n(c(c3)c4c56)c78)c2)cc1]

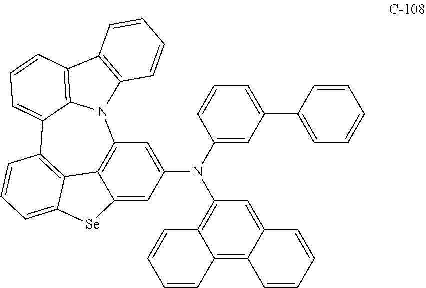 [SMILES: c1ccc(-c2cccc(N(c3cc4[se]c5cccc6c7cccc8c9ccccc9n(c(c3)c4c56)c78)c3cc4ccccc4c4ccccc34)c2)cc1]

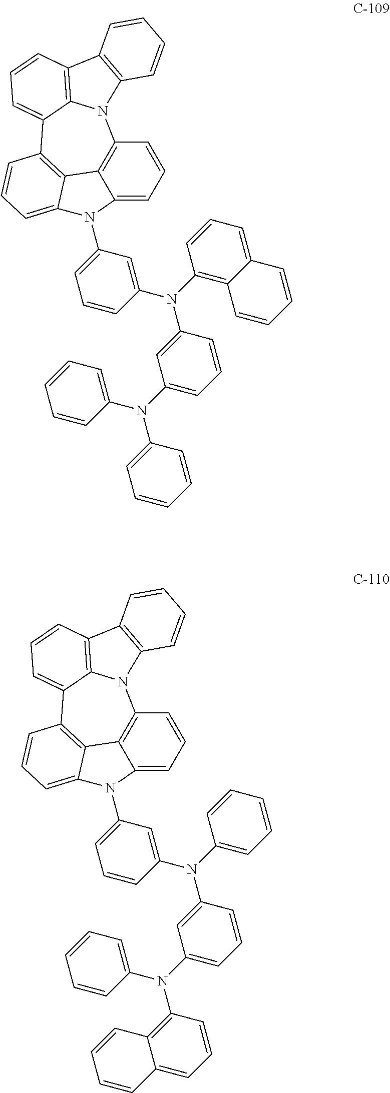 [SMILES: c1ccc(N(c2ccccc2)c2cccc(N(c3cccc(-n4c5cccc6c7cccc8c9ccccc9n(c9cccc4c9c65)c78)c3)c3cccc4ccccc34)c2)cc1 ; c1ccc(N(c2cccc(N(c3ccccc3)c3cccc4ccccc34)c2)c2cccc(-n3c4cccc5c6cccc7c8ccccc8n(c8cccc3c8c54)c67)c2)cc1]

The compound represented by formula 2 may be at least one selected from the group consisting of the following compounds, but is not limited thereto.

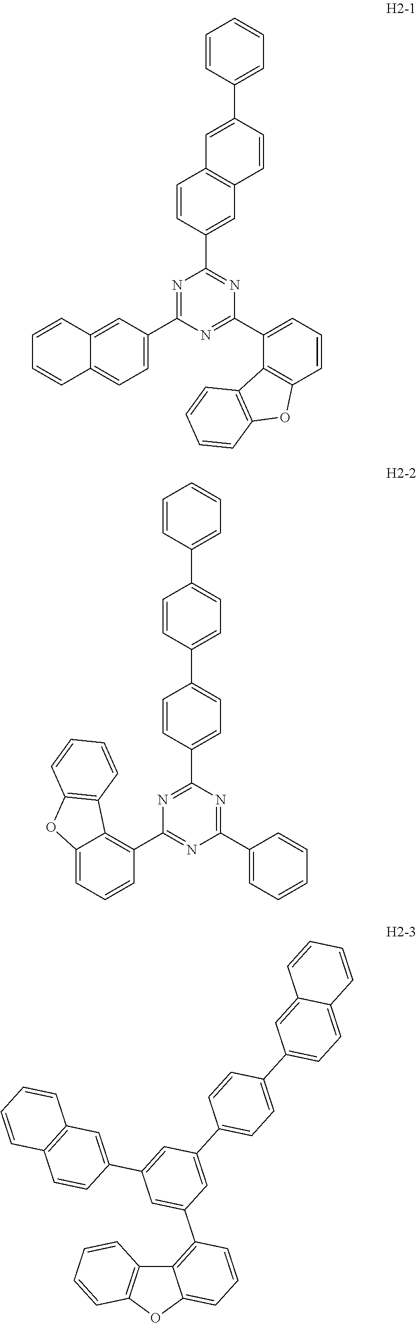

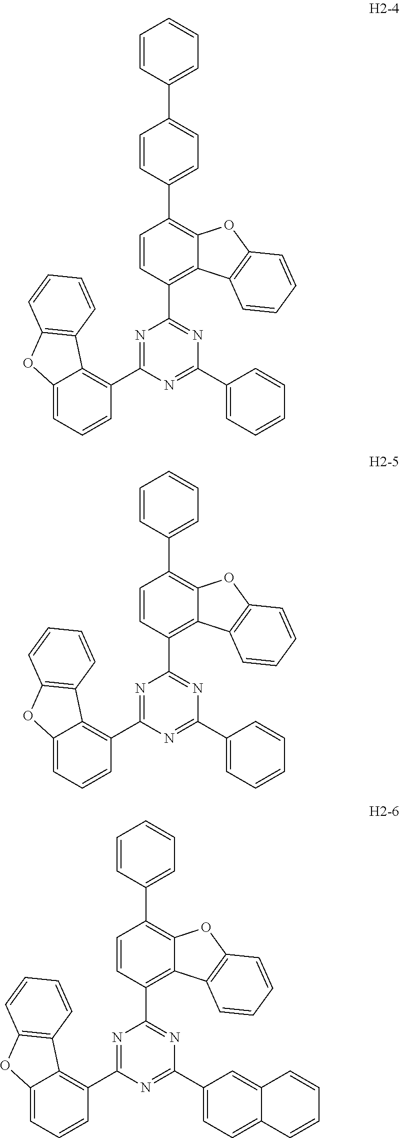 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4cccc5oc6ccccc6c45)n3)c3c2oc2ccccc23)cc1]

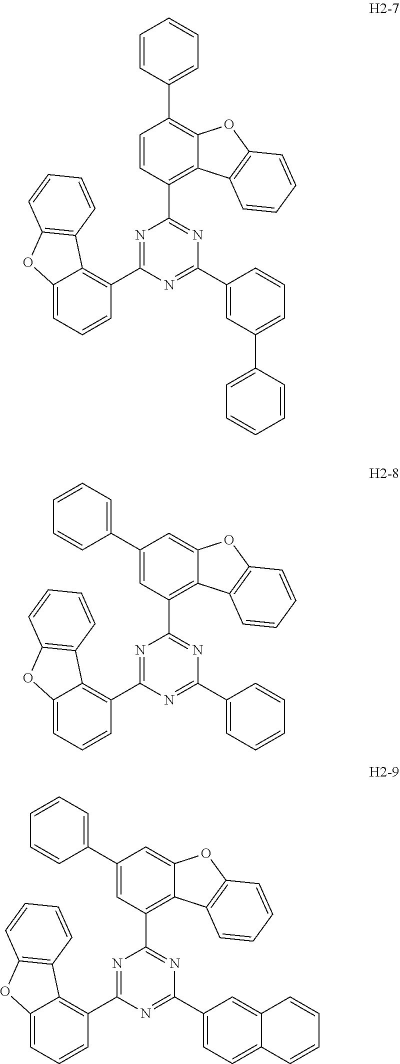 [SMILES: c1ccc(-c2cccc(-c3nc(-c4cccc5oc6ccccc6c45)nc(-c4ccc(-c5ccccc5)c5oc6ccccc6c45)n3)c2)cc1 ; c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccc5ccccc5c4)nc(-c4cccc5oc6ccccc6c45)n3)c3c(c2)oc2ccccc23)cc1]

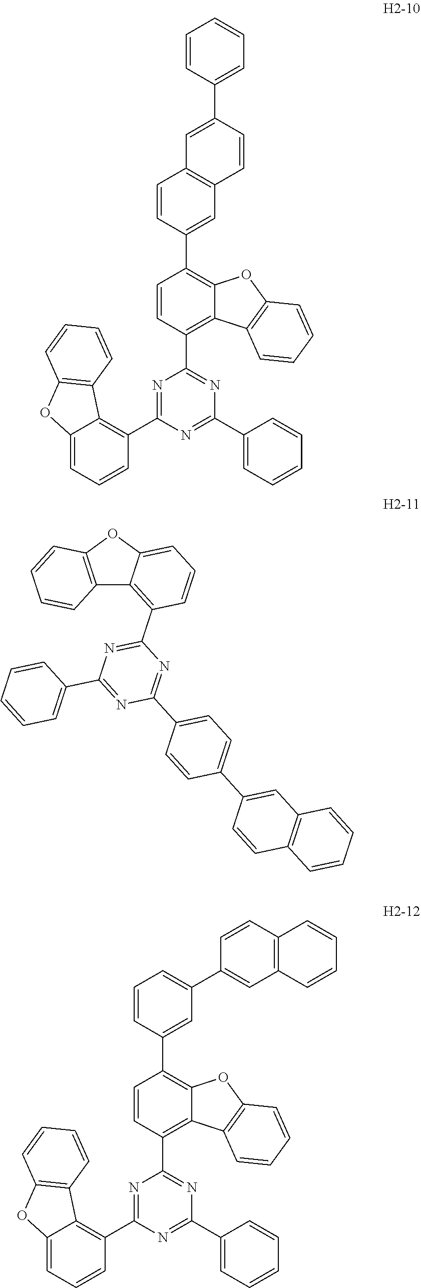 [SMILES: c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3cccc4oc5ccccc5c34)nc(-c3ccc(-c4cccc(-c5ccc6ccccc6c5)c4)c4oc5ccccc5c34)n2)cc1]

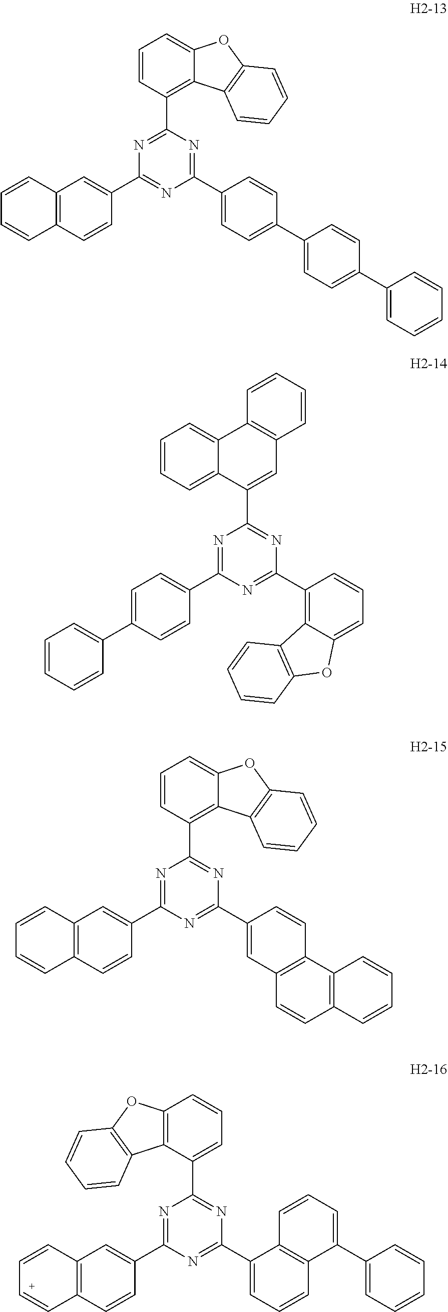 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3nc(-c4cc5ccccc5c5ccccc45)nc(-c4cccc5oc6ccccc6c45)n3)cc2)cc1 ; c1ccc2cc(-c3nc(-c4ccc5c(ccc6ccccc65)c4)nc(-c4cccc5oc6ccccc6c45)n3)ccc2c1 ; [C+]1=Cc2ccc(-c3nc(-c4cccc5c(-c6ccccc6)cccc45)nc(-c4cccc5oc6ccccc6c45)n3)cc2C=C1]

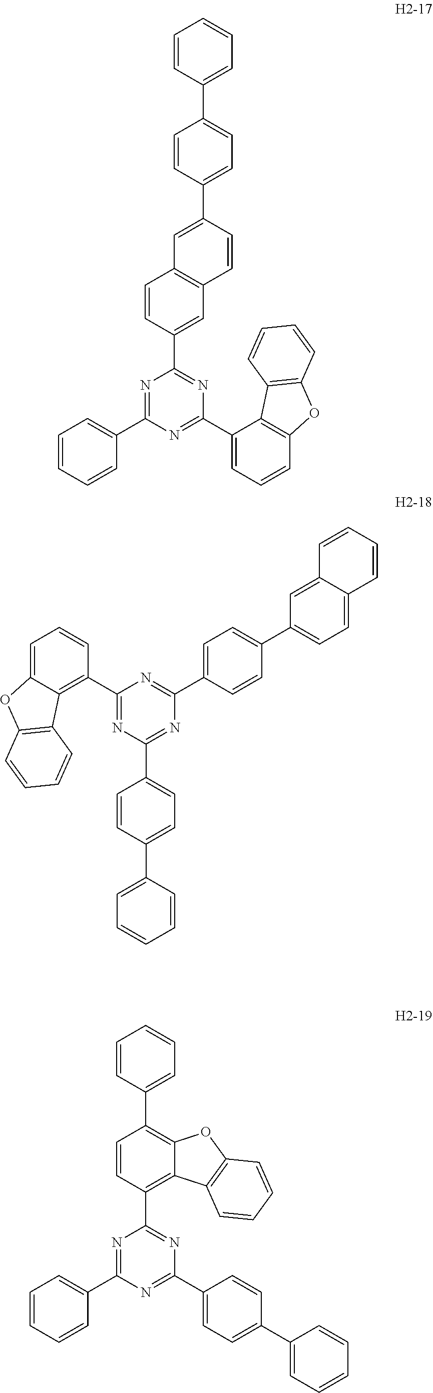 [SMILES: c1ccc(-c2ccc(-c3ccc4cc(-c5nc(-c6ccccc6)nc(-c6cccc7oc8ccccc8c67)n5)ccc4c3)cc2)cc1 ; c1ccc(-c2ccc(-c3nc(-c4ccc(-c5ccc6ccccc6c5)cc4)nc(-c4cccc5oc6ccccc6c45)n3)cc2)cc1 ; c1ccc(-c2ccc(-c3nc(-c4ccccc4)nc(-c4ccc(-c5ccccc5)c5oc6ccccc6c45)n3)cc2)cc1]

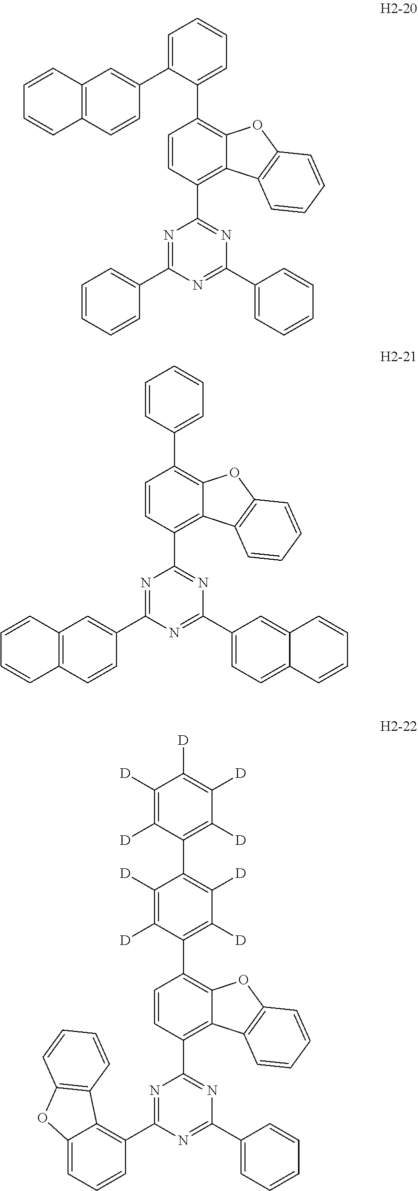 [SMILES: c1ccc(-c2nc(-c3ccccc3)nc(-c3ccc(-c4ccccc4-c4ccc5ccccc5c4)c4oc5ccccc5c34)n2)cc1 ; c1ccc(-c2ccc(-c3nc(-c4ccc5ccccc5c4)nc(-c4ccc5ccccc5c4)n3)c3c2oc2ccccc23)cc1 ; [2H]c1c([2H])c([2H])c(-c2c([2H])c([2H])c(-c3ccc(-c4nc(-c5ccccc5)nc(-c5cccc6oc7ccccc7c56)n4)c4c3oc3ccccc34)c([2H])c2[2H])c([2H])c1[2H]]

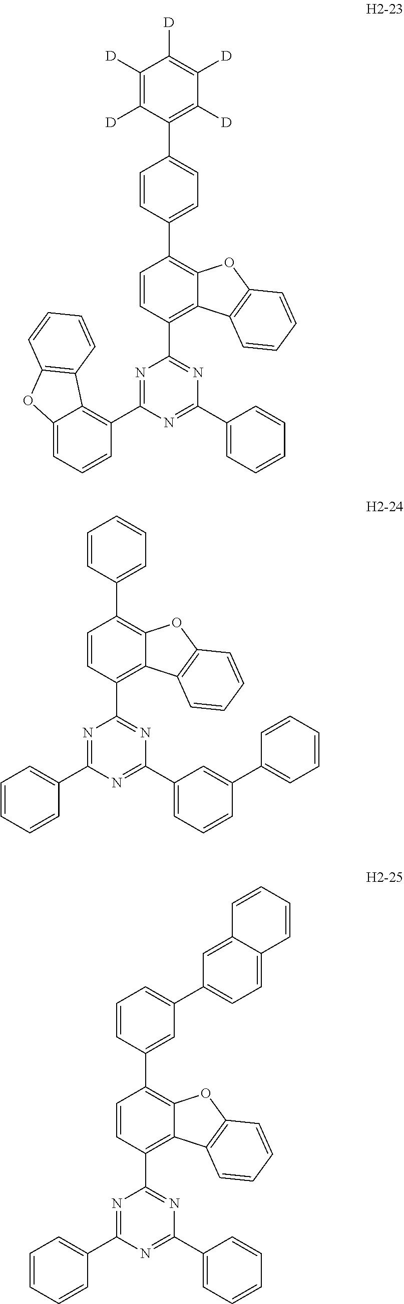 [SMILES: [2H]c1c([2H])c([2H])c(-c2ccc(-c3ccc(-c4nc(-c5ccccc5)nc(-c5cccc6oc7ccccc7c56)n4)c4c3oc3ccccc34)cc2)c([2H])c1[2H] ; c1ccc(-c2cccc(-c3nc(-c4ccccc4)nc(-c4ccc(-c5ccccc5)c5oc6ccccc6c45)n3)c2)cc1 ; c1ccc(-c2nc(-c3ccccc3)nc(-c3ccc(-c4cccc(-c5ccc6ccccc6c5)c4)c4oc5ccccc5c34)n2)cc1]

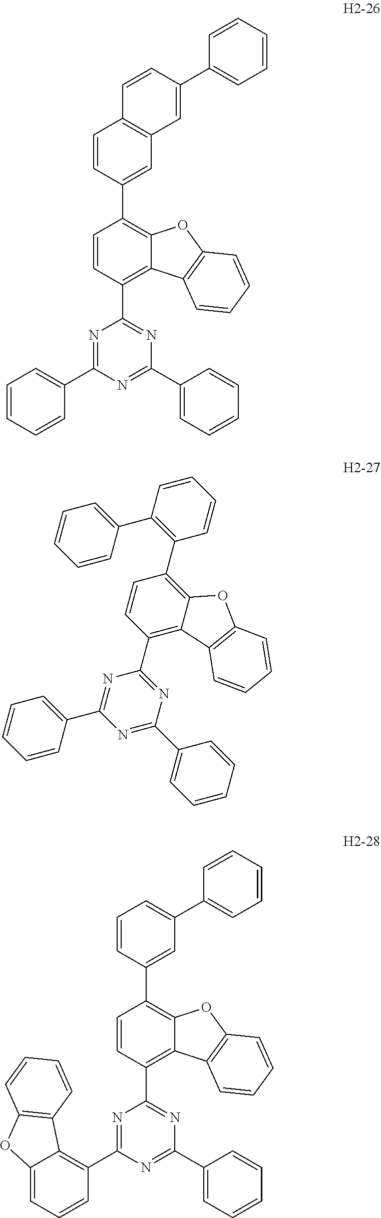

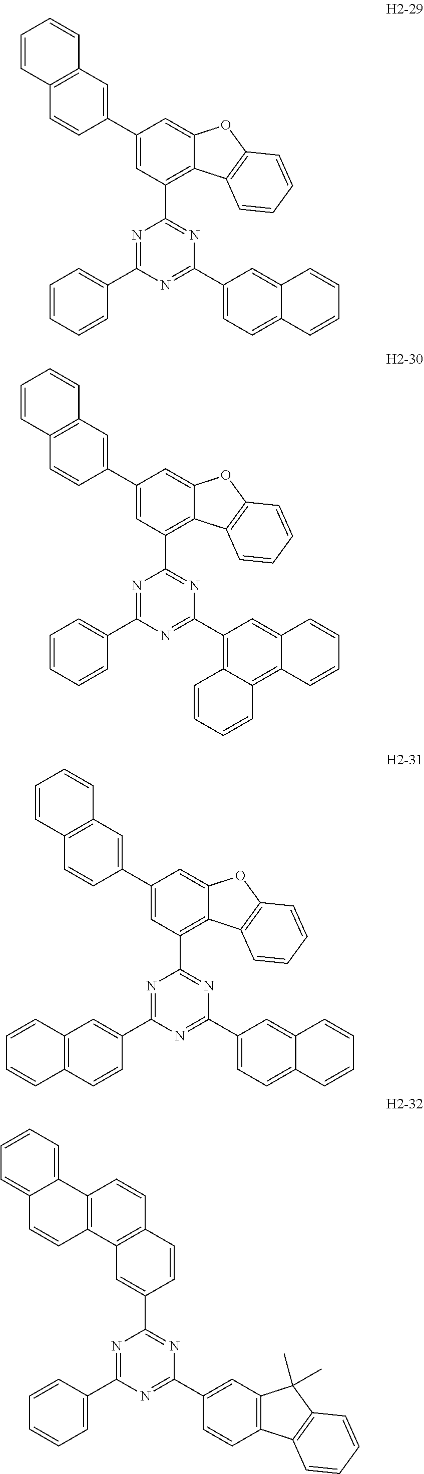

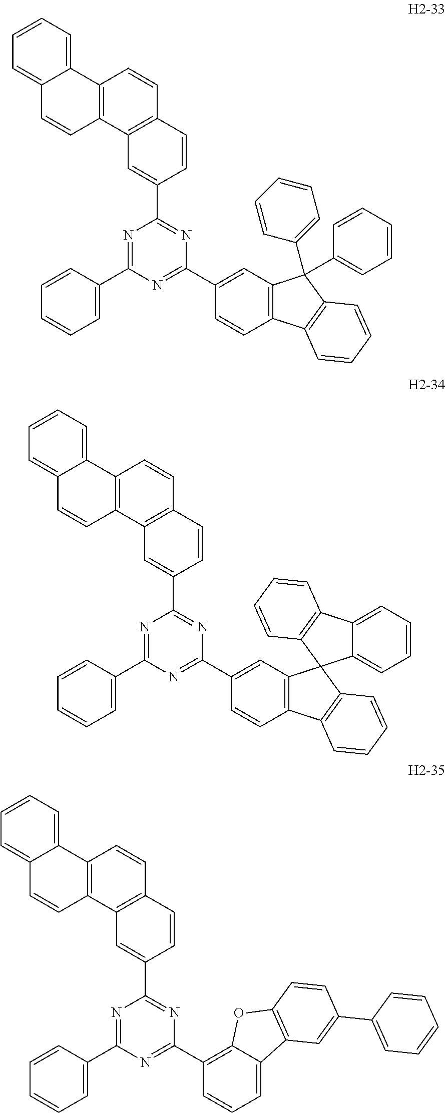 [SMILES: c1ccc(-c2nc(-c3ccc4c(c3)C(c3ccccc3)(c3ccccc3)c3ccccc3-4)nc(-c3ccc4ccc5c6ccccc6ccc5c4c3)n2)cc1 ; c1ccc(-c2nc(-c3ccc4c(c3)C3(c5ccccc5-c5ccccc53)c3ccccc3-4)nc(-c3ccc4ccc5c6ccccc6ccc5c4c3)n2)cc1 ; c1ccc(-c2ccc3oc4c(-c5nc(-c6ccccc6)nc(-c6ccc7ccc8c9ccccc9ccc8c7c6)n5)cccc4c3c2)cc1]

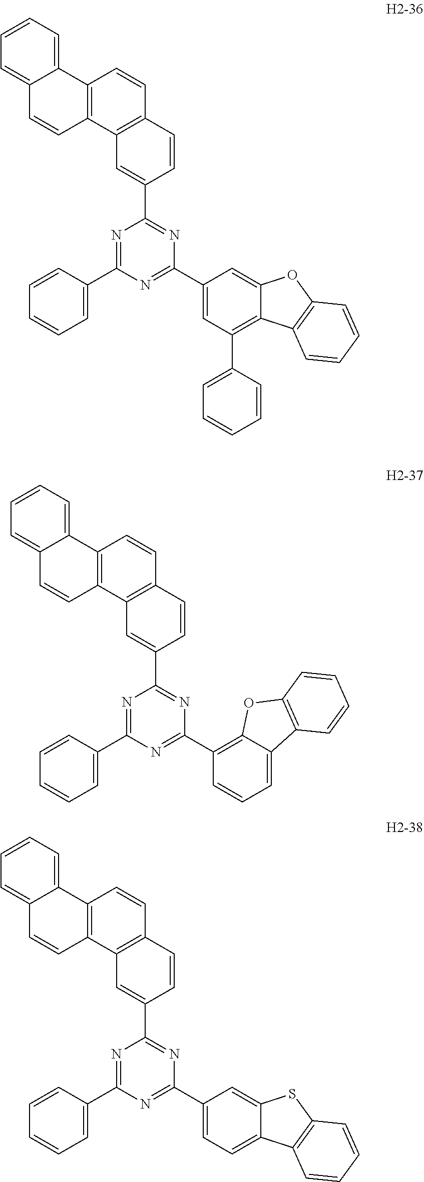 [SMILES: c1ccc(-c2nc(-c3cc(-c4ccccc4)c4c(c3)oc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1]

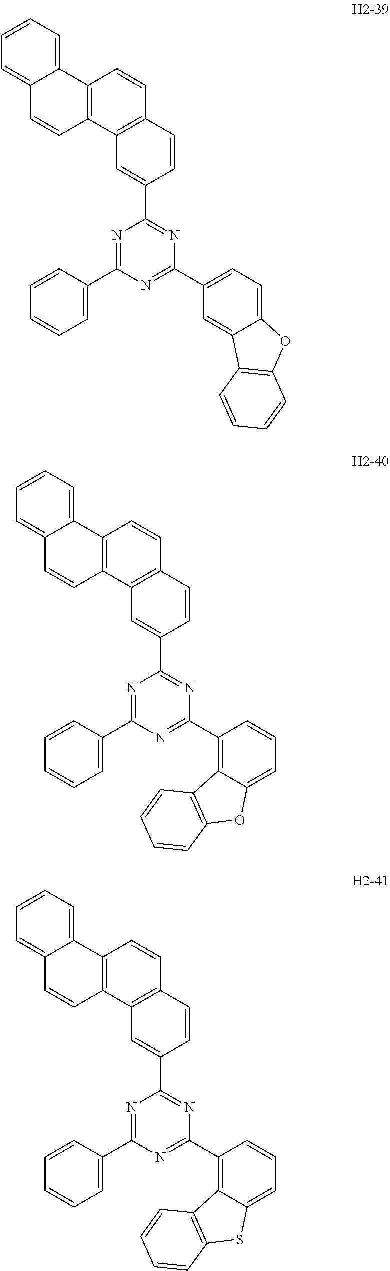 [SMILES: c1ccc(-c2nc(-c3ccc4ccc5c6ccccc6ccc5c4c3)nc(-c3ccc4oc5ccccc5c4c3)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1]

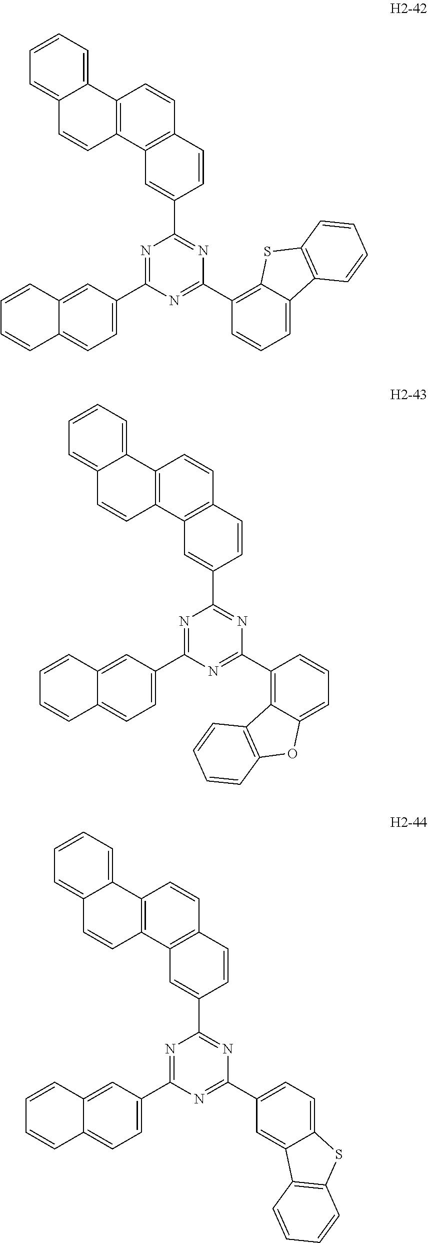 [SMILES: c1ccc2cc(-c3nc(-c4ccc5ccc6c7ccccc7ccc6c5c4)nc(-c4cccc5c4sc4ccccc45)n3)ccc2c1 ; c1ccc2cc(-c3nc(-c4ccc5ccc6c7ccccc7ccc6c5c4)nc(-c4cccc5oc6ccccc6c45)n3)ccc2c1 ; c1ccc2cc(-c3nc(-c4ccc5ccc6c7ccccc7ccc6c5c4)nc(-c4ccc5sc6ccccc6c5c4)n3)ccc2c1]

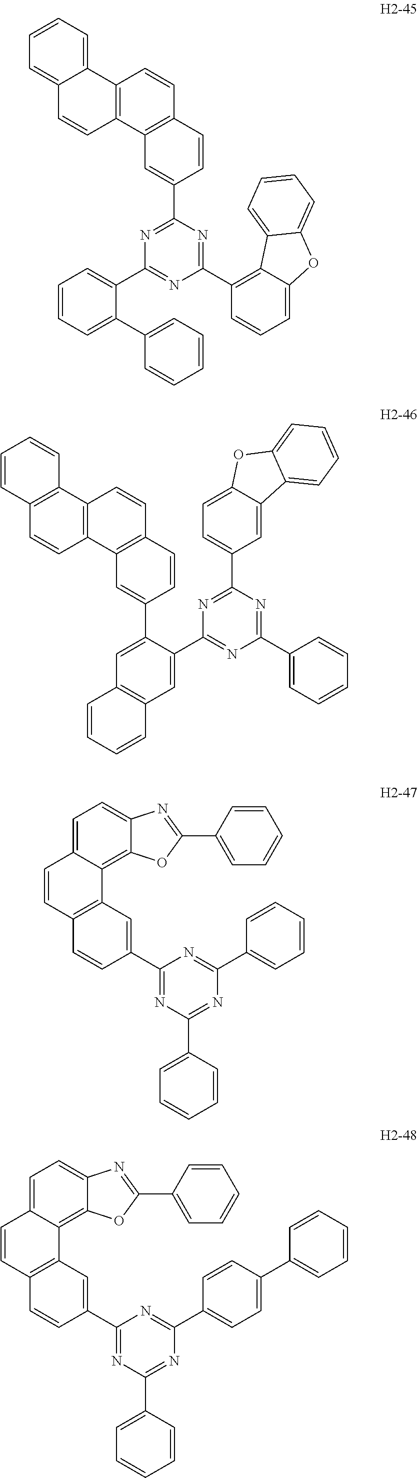

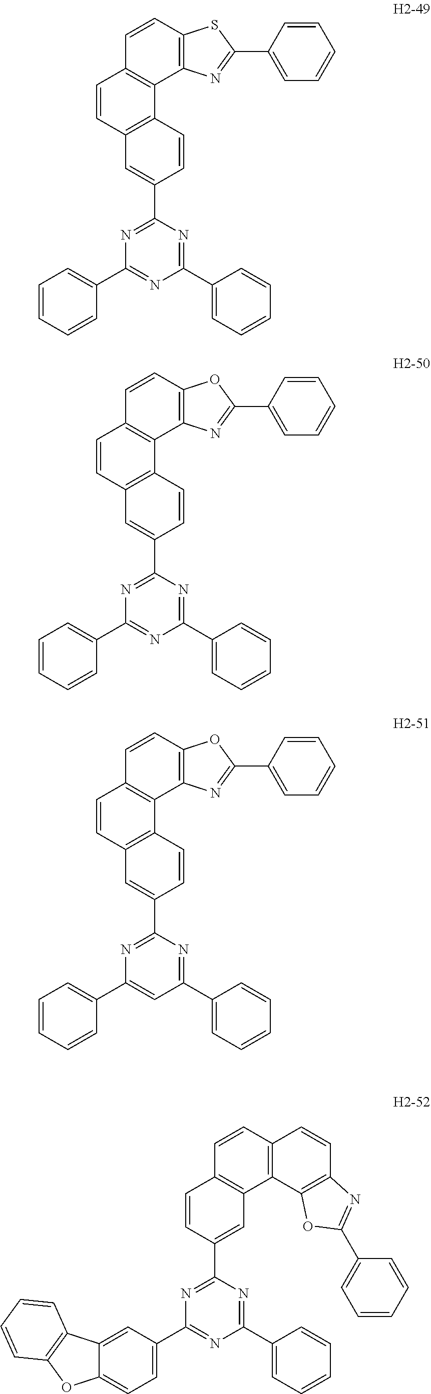 [SMILES: c1ccc(-c2nc(-c3ccccc3)nc(-c3ccc4c(ccc5ccc6sc(-c7ccccc7)nc6c54)c3)n2)cc1 ; c1ccc(-c2nc(-c3ccccc3)nc(-c3ccc4c(ccc5ccc6oc(-c7ccccc7)nc6c54)c3)n2)cc1 ; c1ccc(-c2cc(-c3ccccc3)nc(-c3ccc4c(ccc5ccc6oc(-c7ccccc7)nc6c54)c3)n2)cc1 ; c1ccc(-c2nc(-c3ccc4oc5ccccc5c4c3)nc(-c3ccc4ccc5ccc6nc(-c7ccccc7)oc6c5c4c3)n2)cc1]

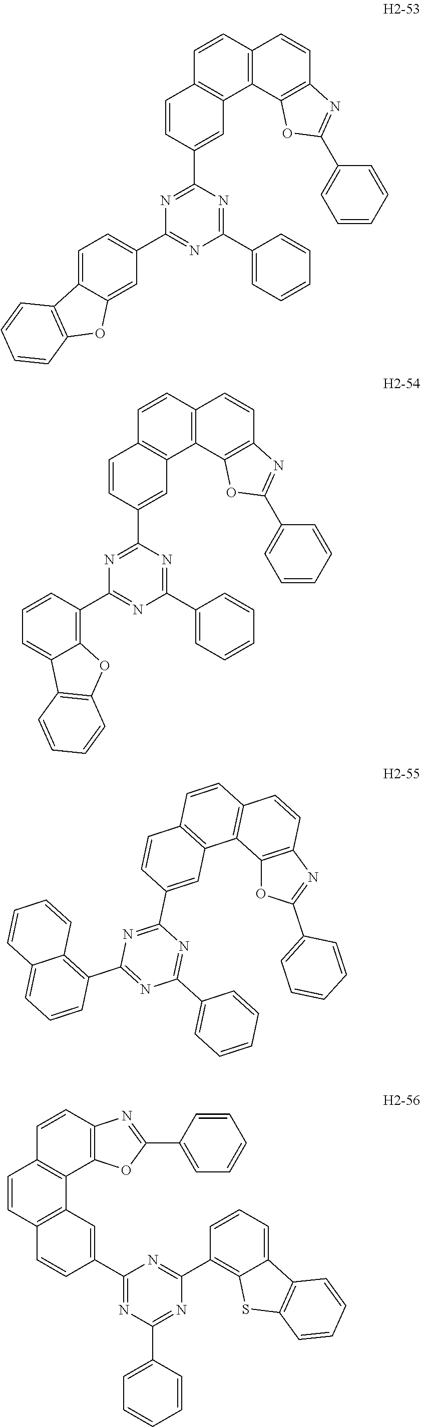 [SMILES: c1ccc(-c2nc(-c3ccc4c(c3)oc3ccccc34)nc(-c3ccc4ccc5ccc6nc(-c7ccccc7)oc6c5c4c3)n2)cc1 ; c1ccc(-c2nc(-c3ccc4ccc5ccc6nc(-c7ccccc7)oc6c5c4c3)nc(-c3cccc4c3oc3ccccc34)n2)cc1 ; c1ccc(-c2nc(-c3ccc4ccc5ccc6nc(-c7ccccc7)oc6c5c4c3)nc(-c3cccc4ccccc34)n2)cc1 ; c1ccc(-c2nc(-c3ccc4ccc5ccc6nc(-c7ccccc7)oc6c5c4c3)nc(-c3cccc4c3sc3ccccc34)n2)cc1]

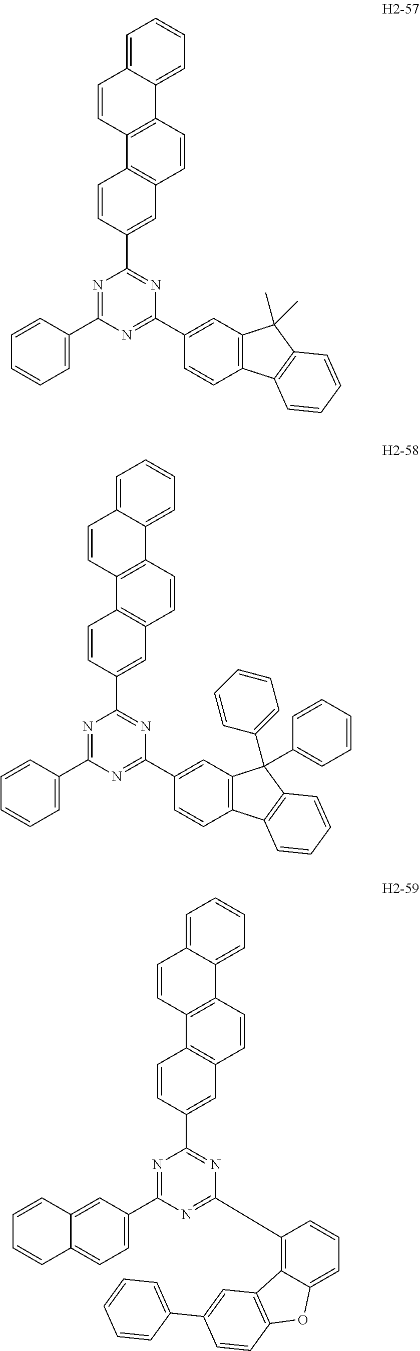 [SMILES: CC1(C)c2ccccc2-c2ccc(-c3nc(-c4ccccc4)nc(-c4ccc5c(ccc6c7ccccc7ccc56)c4)n3)cc21 ; c1ccc(-c2nc(-c3ccc4c(c3)C(c3ccccc3)(c3ccccc3)c3ccccc3-4)nc(-c3ccc4c(ccc5c6ccccc6ccc45)c3)n2)cc1 ; c1ccc(-c2ccc3oc4cccc(-c5nc(-c6ccc7ccccc7c6)nc(-c6ccc7c(ccc8c9ccccc9ccc78)c6)n5)c4c3c2)cc1]

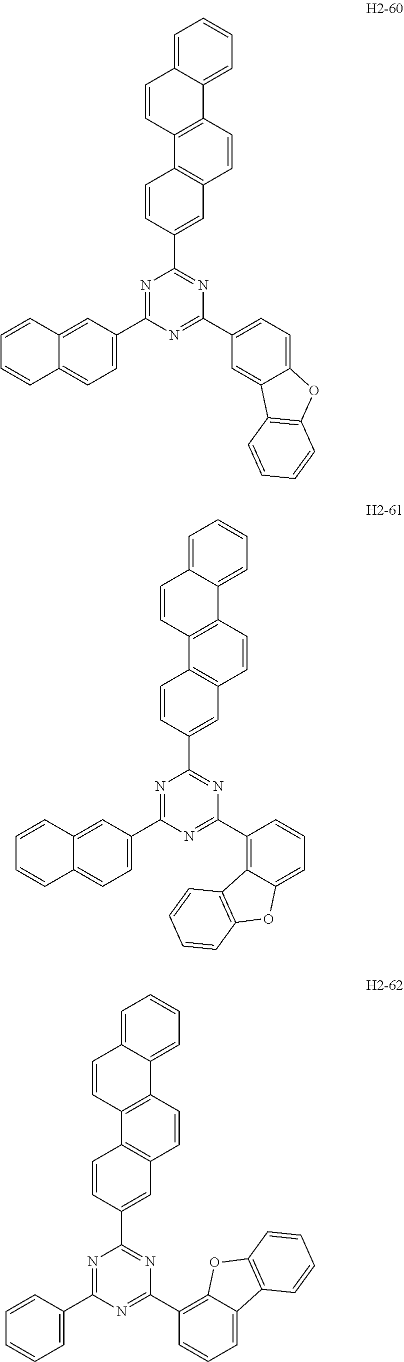 [SMILES: c1ccc2cc(-c3nc(-c4ccc5c(ccc6c7ccccc7ccc56)c4)nc(-c4ccc5oc6ccccc6c5c4)n3)ccc2c1 ; c1ccc2cc(-c3nc(-c4ccc5c(ccc6c7ccccc7ccc56)c4)nc(-c4cccc5oc6ccccc6c45)n3)ccc2c1 ; c1ccc(-c2nc(-c3ccc4c(ccc5c6ccccc6ccc45)c3)nc(-c3cccc4c3oc3ccccc34)n2)cc1]

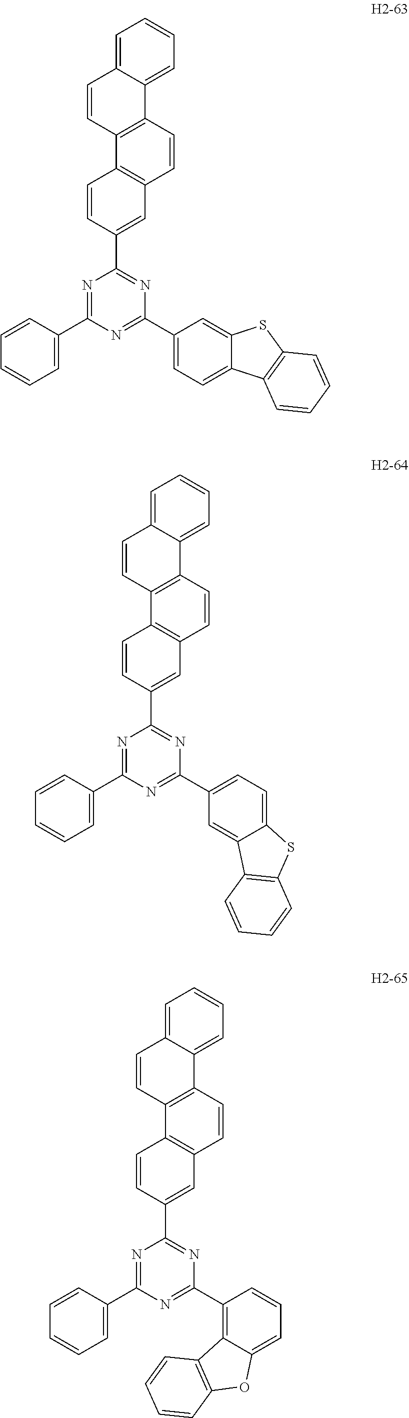 [SMILES: c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1 ; c1ccc(-c2nc(-c3ccc4c(ccc5c6ccccc6ccc45)c3)nc(-c3cccc4oc5ccccc5c34)n2)cc1]

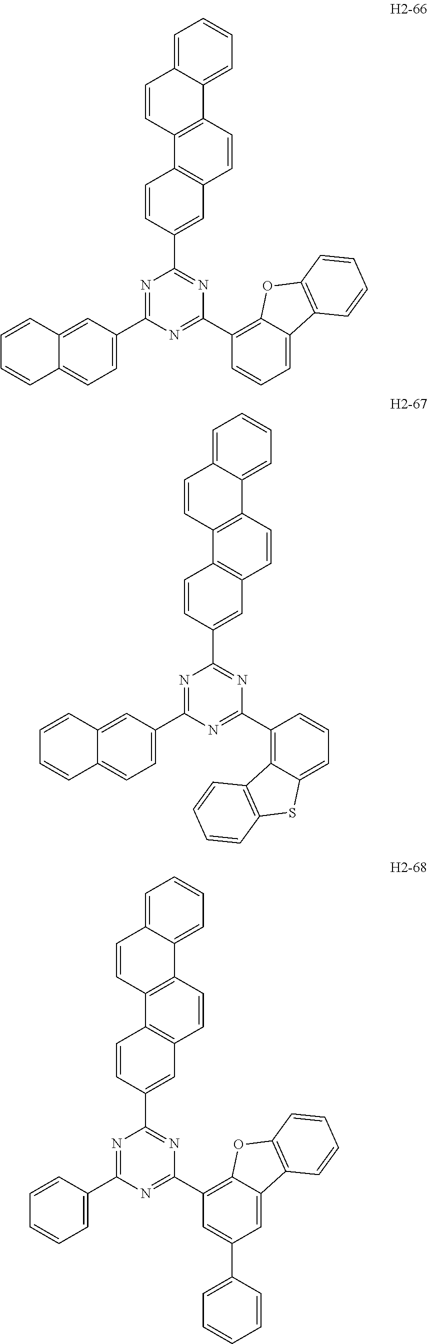

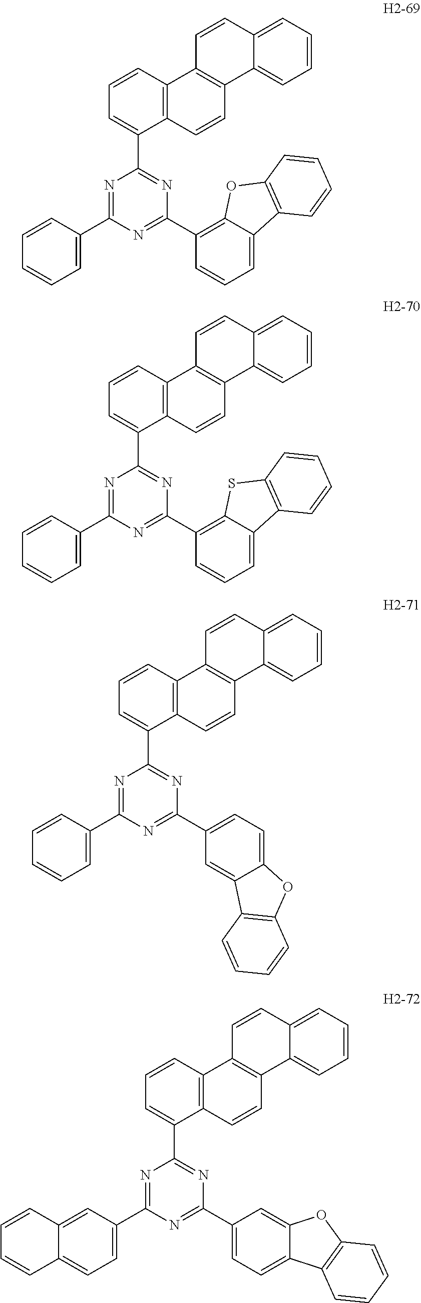

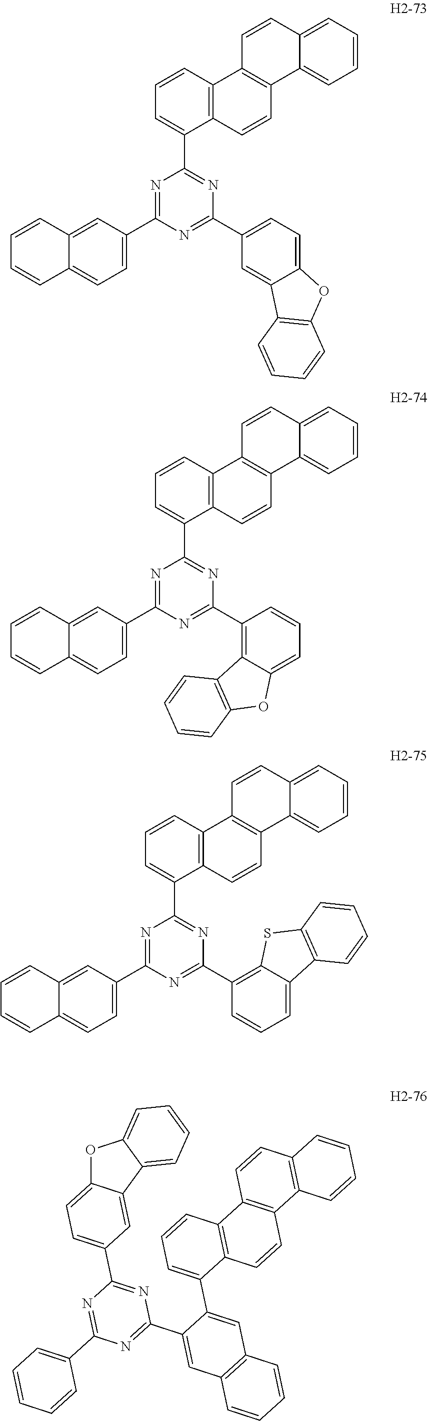

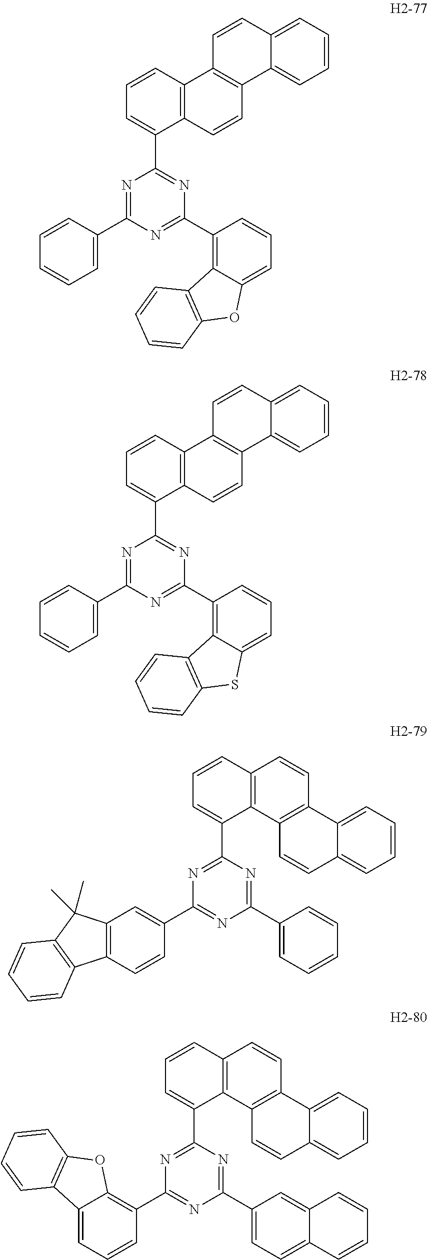 [SMILES: c1ccc(-c2nc(-c3cccc4c3ccc3c5ccccc5ccc43)nc(-c3cccc4oc5ccccc5c34)n2)cc1 ; c1ccc(-c2nc(-c3cccc4c3ccc3c5ccccc5ccc43)nc(-c3cccc4sc5ccccc5c34)n2)cc1 ; CC1(C)c2ccccc2-c2ccc(-c3nc(-c4ccccc4)nc(-c4cccc5ccc6c7ccccc7ccc6c45)n3)cc21 ; c1ccc2cc(-c3nc(-c4cccc5c4oc4ccccc45)nc(-c4cccc5ccc6c7ccccc7ccc6c45)n3)ccc2c1]

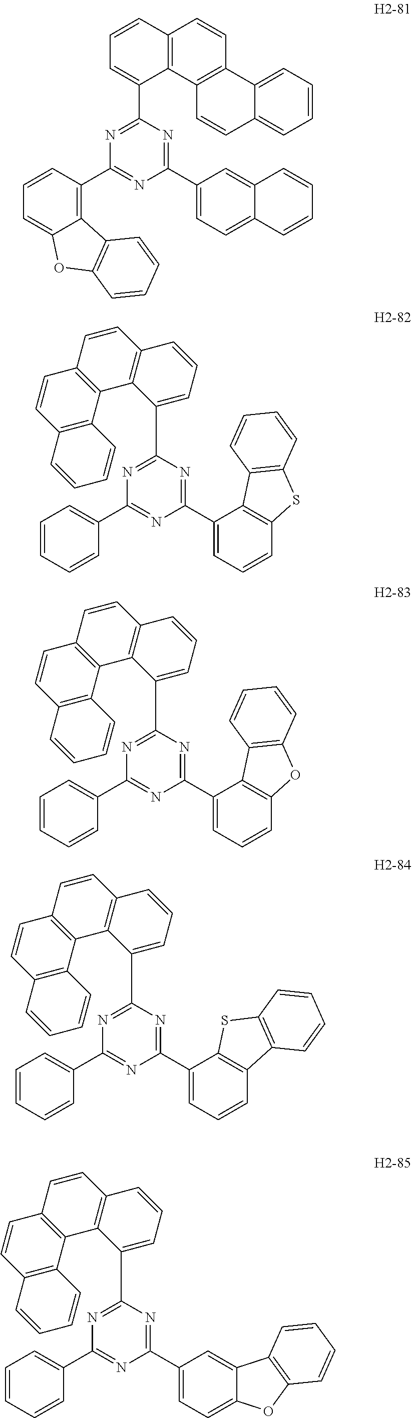

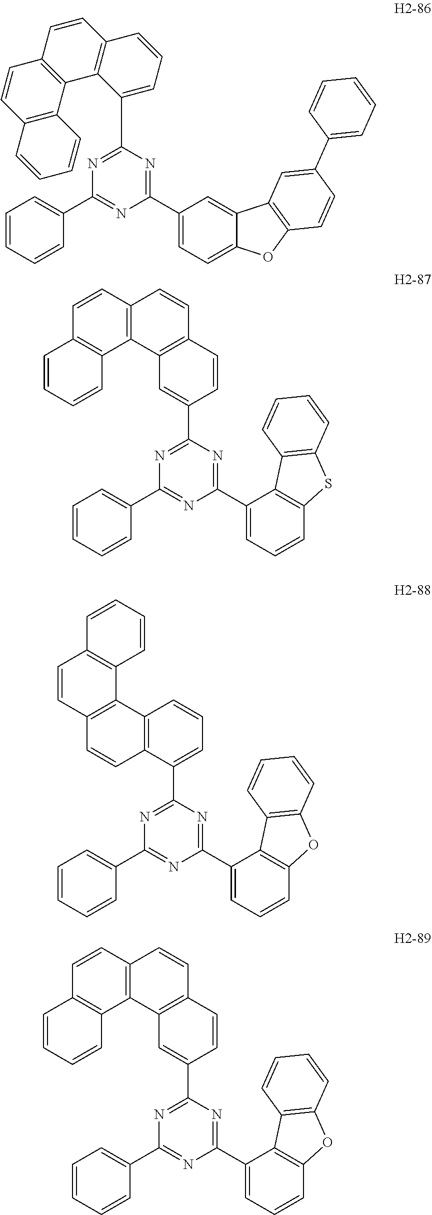

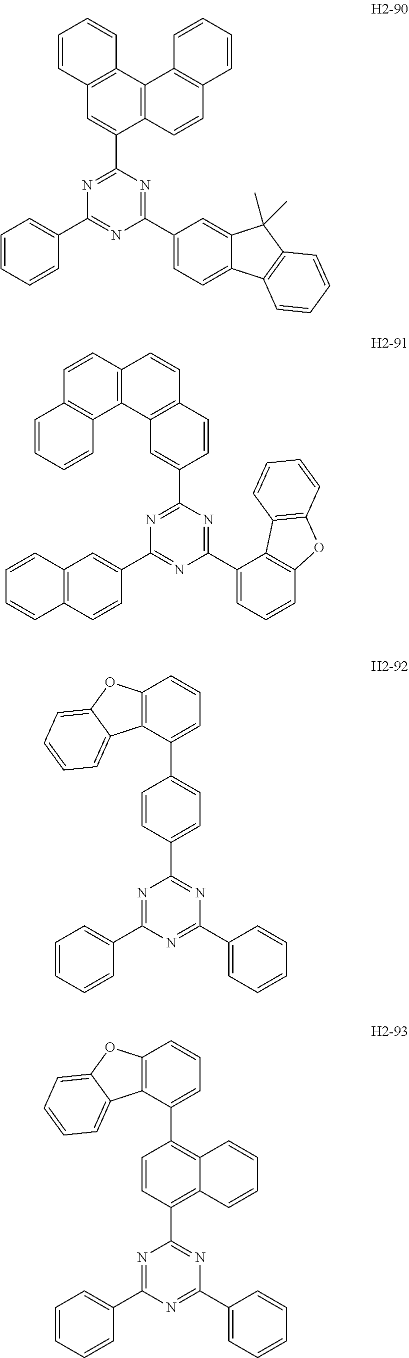 [SMILES: CC1(C)c2ccccc2-c2ccc(-c3nc(-c4ccccc4)nc(-c4cc5ccccc5c5c4ccc4ccccc45)n3)cc21 ; c1ccc2cc(-c3nc(-c4ccc5ccc6ccc7ccccc7c6c5c4)nc(-c4cccc5oc6ccccc6c45)n3)ccc2c1 ; c1ccc(-c2nc(-c3ccccc3)nc(-c3ccc(-c4cccc5oc6ccccc6c45)cc3)n2)cc1 ; c1ccc(-c2nc(-c3ccccc3)nc(-c3ccc(-c4cccc5oc6ccccc6c45)c4ccccc34)n2)cc1]

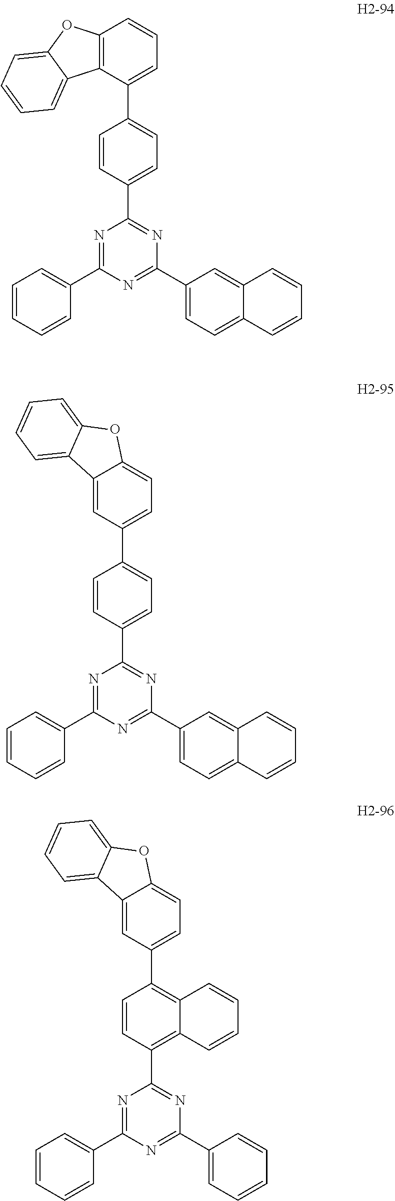 [SMILES: c1ccc(-c2nc(-c3ccc(-c4cccc5oc6ccccc6c45)cc3)nc(-c3ccc4ccccc4c3)n2)cc1 ; c1ccc(-c2nc(-c3ccc(-c4ccc5oc6ccccc6c5c4)cc3)nc(-c3ccc4ccccc4c3)n2)cc1 ; c1ccc(-c2nc(-c3ccccc3)nc(-c3ccc(-c4ccc5oc6ccccc6c5c4)c4ccccc34)n2)cc1]

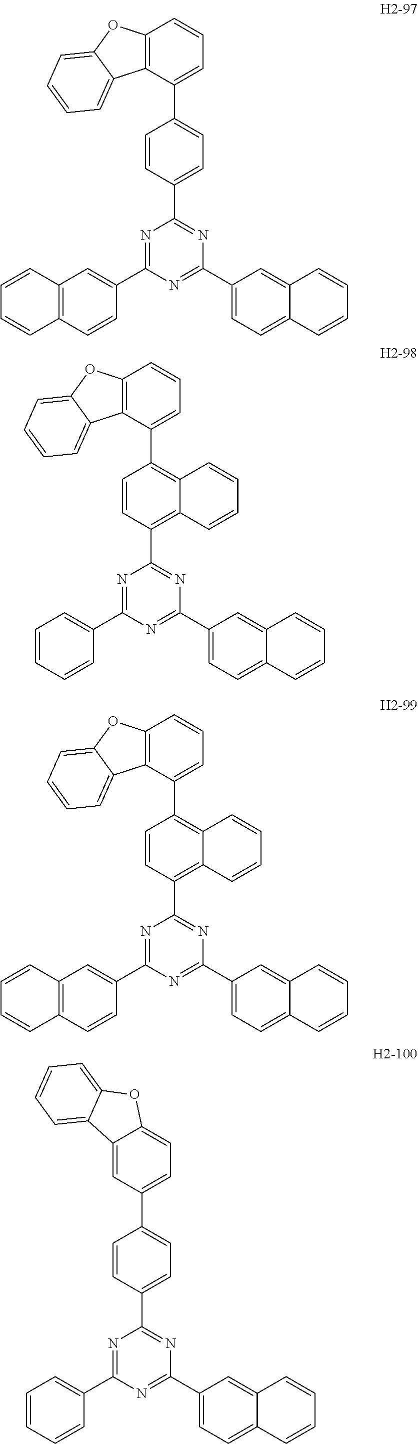

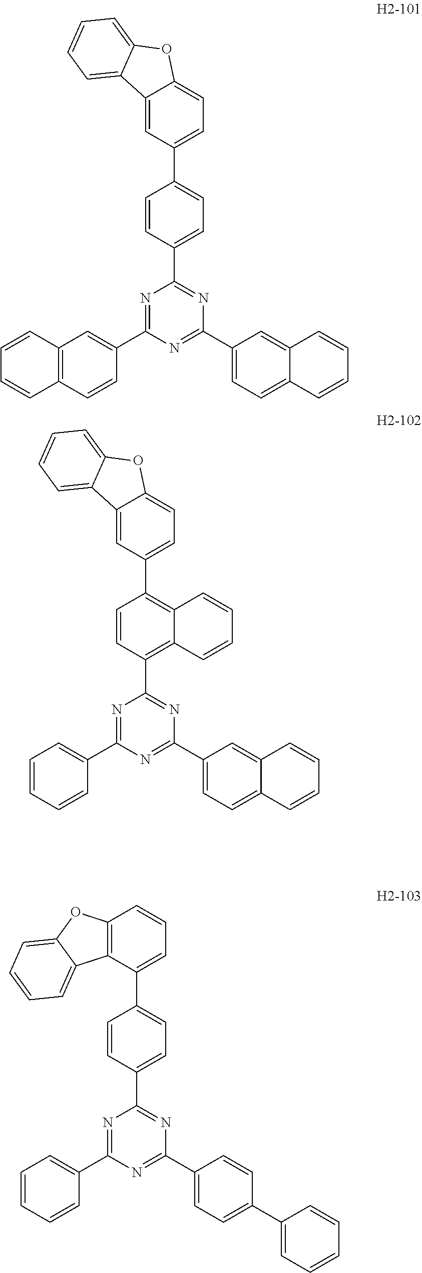

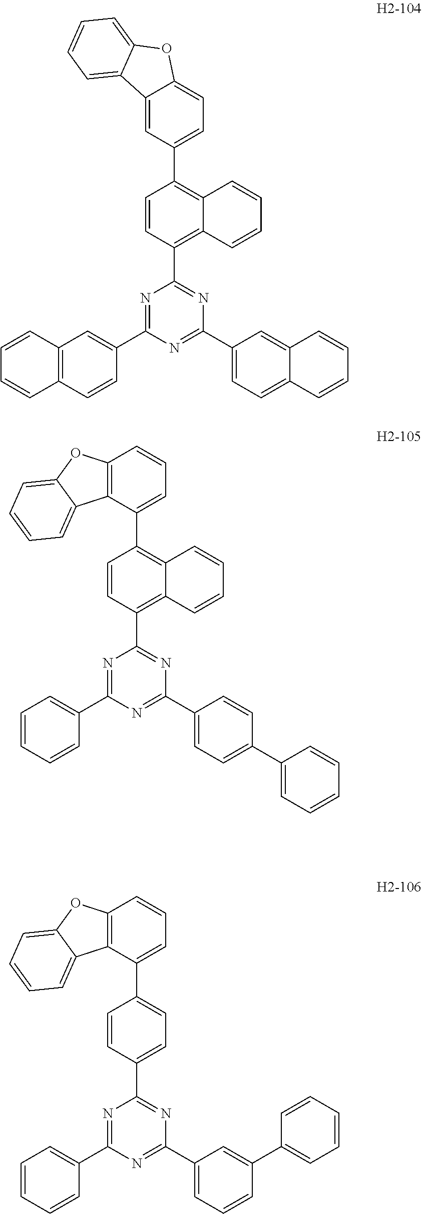 [SMILES: c1ccc2cc(-c3nc(-c4ccc5ccccc5c4)nc(-c4ccc(-c5ccc6oc7ccccc7c6c5)c5ccccc45)n3)ccc2c1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1]

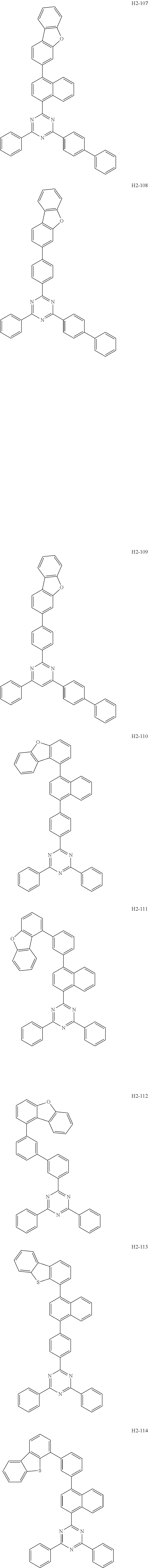

[chemical structure]

[chemical structure]

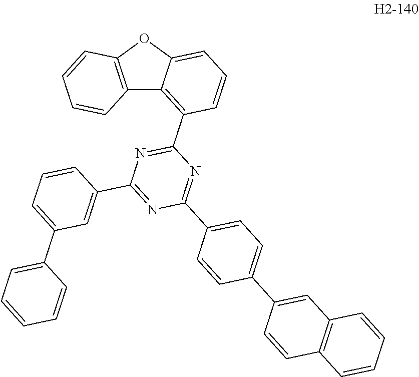 [SMILES: c1ccc(-c2cccc(-c3nc(-c4ccc(-c5ccc6ccccc6c5)cc4)nc(-c4cccc5oc6ccccc6c45)n3)c2)cc1]

[chemical structure]

[chemical structure]

[chemical structure]

[chemical structure]

[chemical structure]

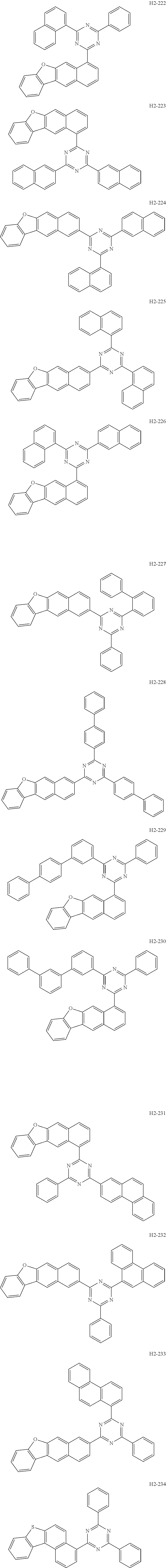

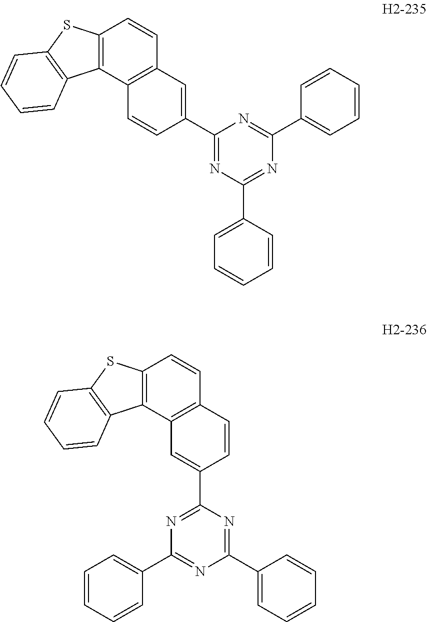 [SMILES: c1ccc(-c2nc(-c3ccccc3)nc(-c3ccc4c(ccc5sc6ccccc6c54)c3)n2)cc1 ; c1ccc(-c2nc(-c3ccccc3)nc(-c3ccc4ccc5sc6ccccc6c5c4c3)n2)cc1]

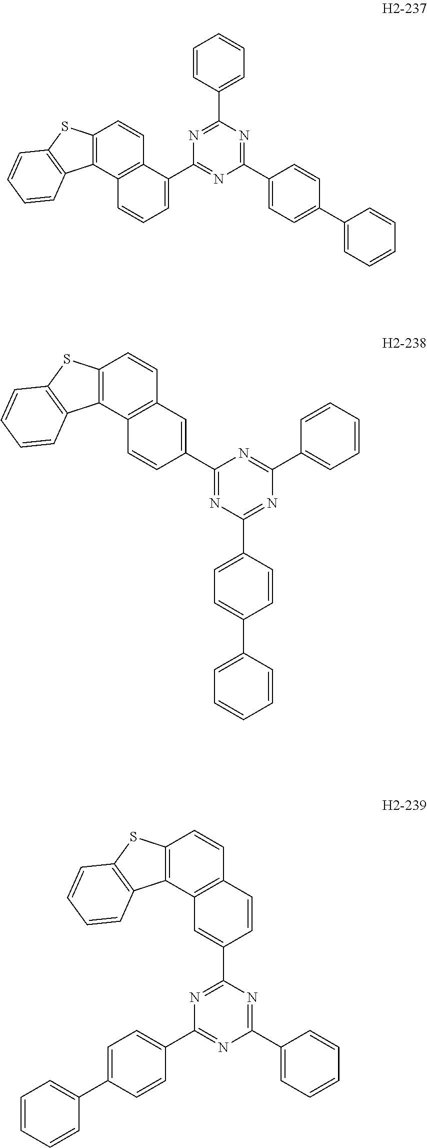 [SMILES: c1ccc(-c2ccc(-c3nc(-c4ccccc4)nc(-c4cccc5c4ccc4sc6ccccc6c45)n3)cc2)cc1 ; c1ccc(-c2ccc(-c3nc(-c4ccccc4)nc(-c4ccc5c(ccc6sc7ccccc7c65)c4)n3)cc2)cc1 ; c1ccc(-c2ccc(-c3nc(-c4ccccc4)nc(-c4ccc5ccc6sc7ccccc7c6c5c4)n3)cc2)cc1]

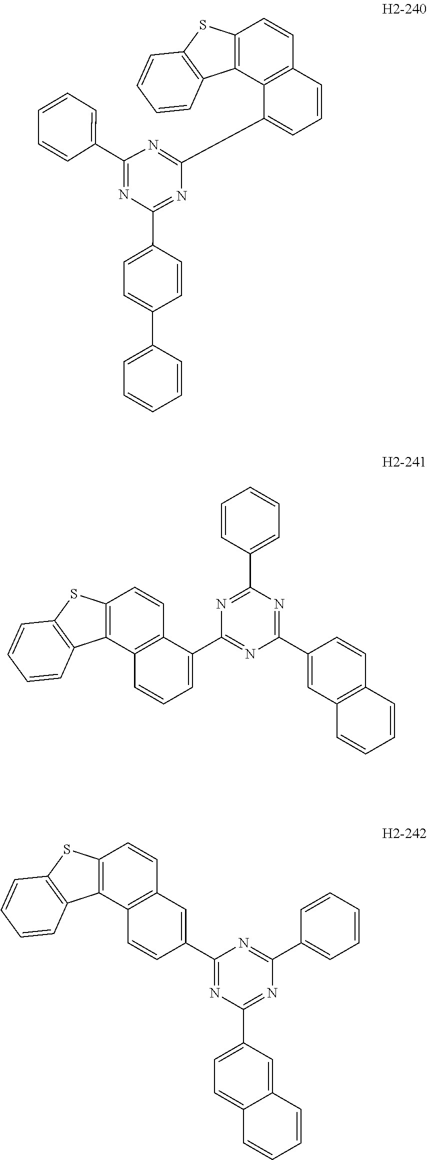 [SMILES: c1ccc(-c2ccc(-c3nc(-c4ccccc4)nc(-c4cccc5ccc6sc7ccccc7c6c45)n3)cc2)cc1 ; c1ccc(-c2nc(-c3ccc4ccccc4c3)nc(-c3cccc4c3ccc3sc5ccccc5c34)n2)cc1 ; c1ccc(-c2nc(-c3ccc4ccccc4c3)nc(-c3ccc4c(ccc5sc6ccccc6c54)c3)n2)cc1]

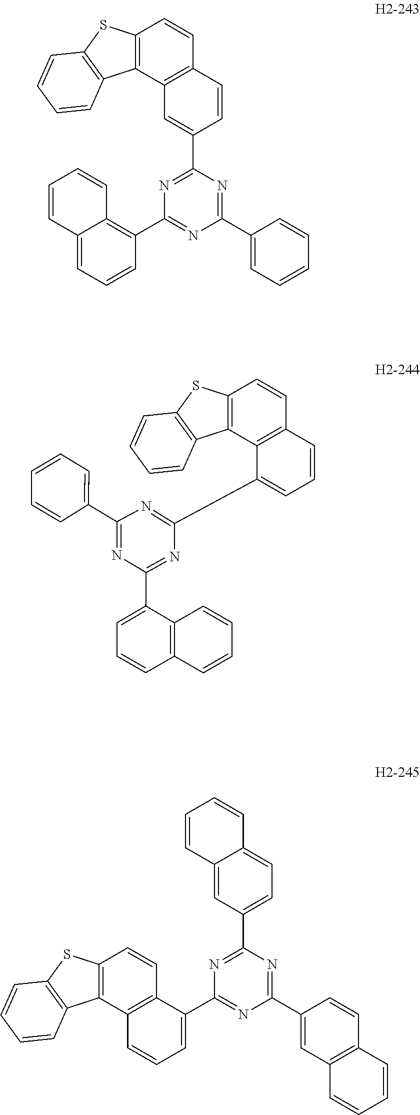 [SMILES: c1ccc(-c2nc(-c3ccc4ccc5sc6ccccc6c5c4c3)nc(-c3cccc4ccccc34)n2)cc1 ; c1ccc(-c2nc(-c3cccc4ccccc34)nc(-c3cccc4ccc5sc6ccccc6c5c34)n2)cc1 ; c1ccc2cc(-c3nc(-c4ccc5ccccc5c4)nc(-c4cccc5c4ccc4sc6ccccc6c45)n3)ccc2c1]

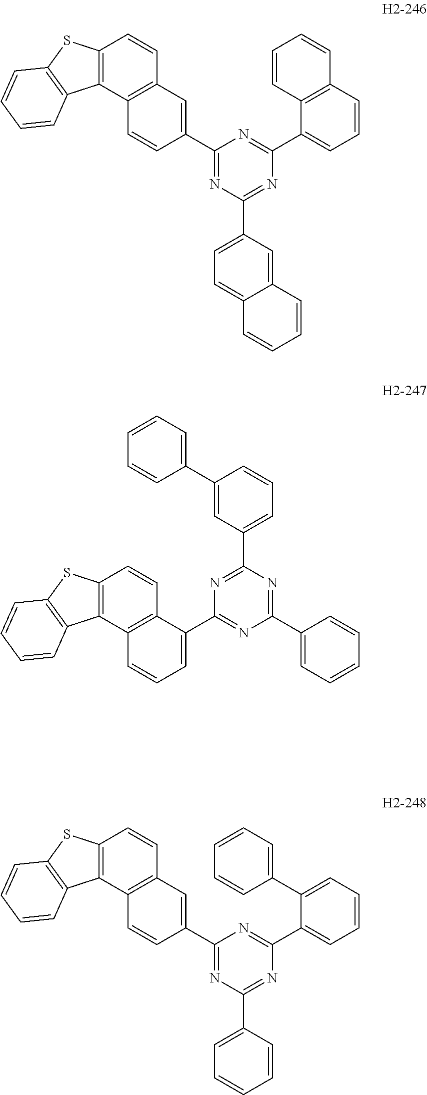 [SMILES: c1ccc2cc(-c3nc(-c4ccc5c(ccc6sc7ccccc7c65)c4)nc(-c4cccc5ccccc45)n3)ccc2c1 ; c1ccc(-c2cccc(-c3nc(-c4ccccc4)nc(-c4cccc5c4ccc4sc6ccccc6c45)n3)c2)cc1 ; c1ccc(-c2nc(-c3ccc4c(ccc5sc6ccccc6c54)c3)nc(-c3ccccc3-c3ccccc3)n2)cc1]

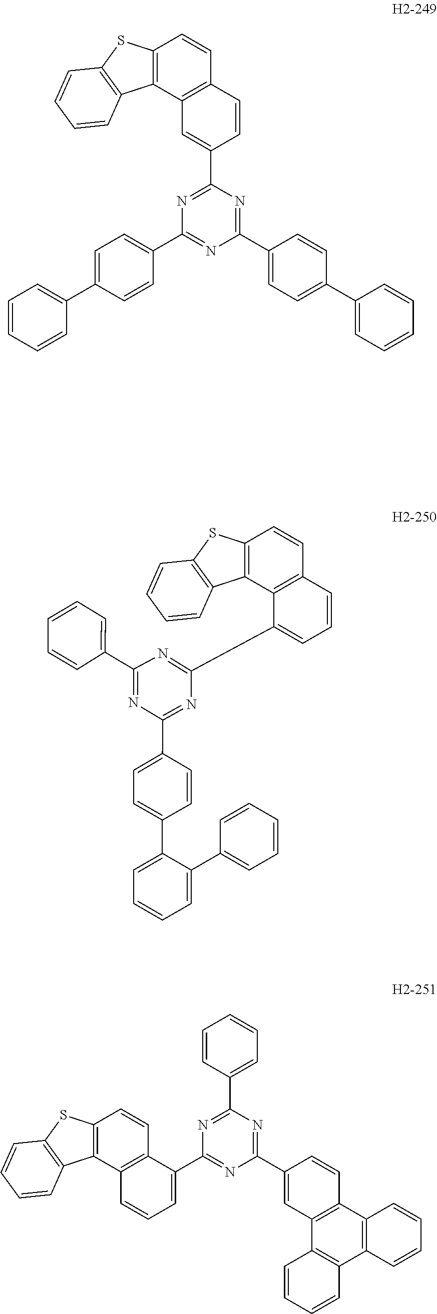 [SMILES: c1ccc(-c2ccc(-c3nc(-c4ccc(-c5ccccc5)cc4)nc(-c4ccc5ccc6sc7ccccc7c6c5c4)n3)cc2)cc1 ; c1ccc(-c2nc(-c3ccc(-c4ccccc4-c4ccccc4)cc3)nc(-c3cccc4ccc5sc6ccccc6c5c34)n2)cc1 ; c1ccc(-c2nc(-c3ccc4c5ccccc5c5ccccc5c4c3)nc(-c3cccc4c3ccc3sc5ccccc5c34)n2)cc1]

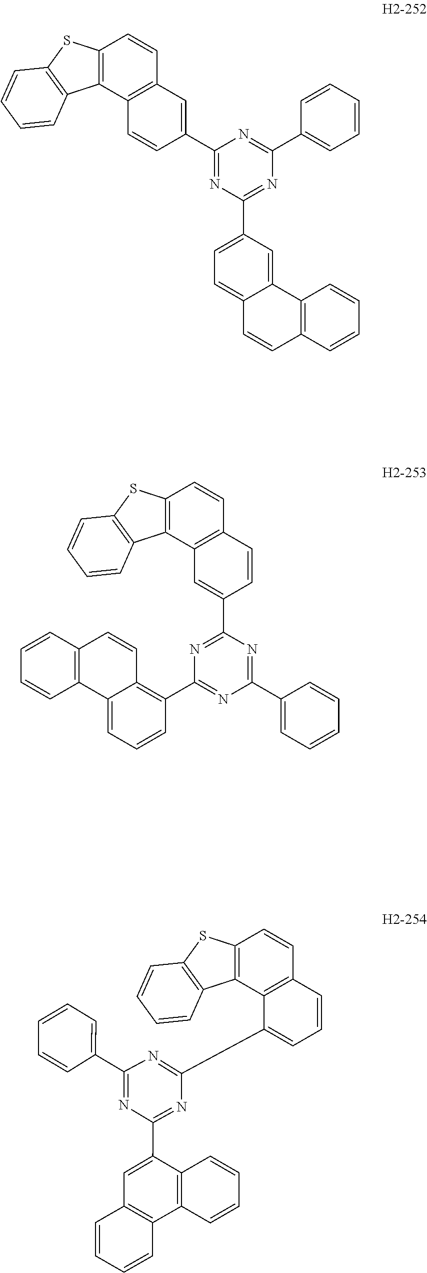 [SMILES: c1ccc(-c2nc(-c3ccc4c(ccc5sc6ccccc6c54)c3)nc(-c3ccc4ccc5ccccc5c4c3)n2)cc1 ; c1ccc(-c2nc(-c3ccc4ccc5sc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5sc6ccccc6c5c34)n2)cc1]

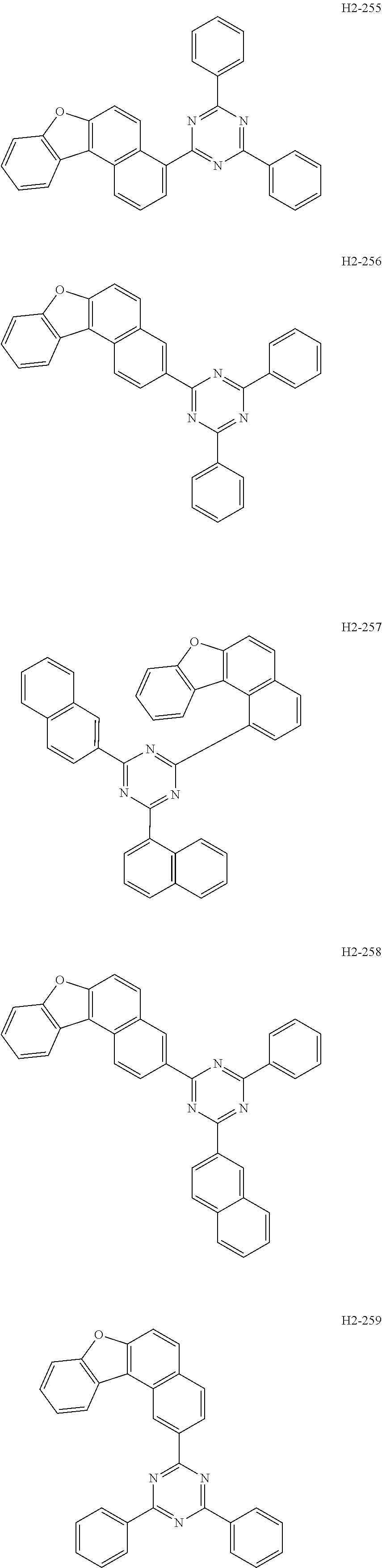 [SMILES: c1ccc(-c2nc(-c3ccccc3)nc(-c3cccc4c3ccc3oc5ccccc5c34)n2)cc1 ; c1ccc(-c2nc(-c3ccccc3)nc(-c3ccc4c(ccc5oc6ccccc6c54)c3)n2)cc1 ; c1ccc2cc(-c3nc(-c4cccc5ccccc45)nc(-c4cccc5ccc6oc7ccccc7c6c45)n3)ccc2c1 ; c1ccc(-c2nc(-c3ccc4ccccc4c3)nc(-c3ccc4c(ccc5oc6ccccc6c54)c3)n2)cc1 ; c1ccc(-c2nc(-c3ccccc3)nc(-c3ccc4ccc5oc6ccccc6c5c4c3)n2)cc1]

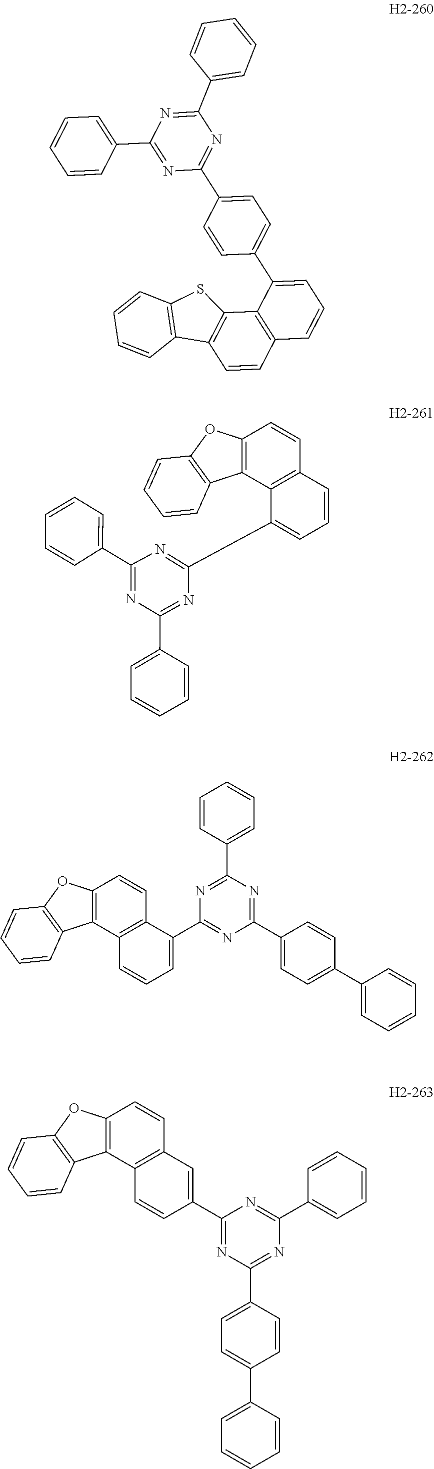 [SMILES: c1ccc(-c2nc(-c3ccccc3)nc(-c3ccc(-c4cccc5ccc6c7ccccc7sc6c45)cc3)n2)cc1 ; c1ccc(-c2nc(-c3ccccc3)nc(-c3cccc4ccc5oc6ccccc6c5c34)n2)cc1 ; c1ccc(-c2ccc(-c3nc(-c4ccccc4)nc(-c4cccc5c4ccc4oc6ccccc6c45)n3)cc2)cc1 ; c1ccc(-c2ccc(-c3nc(-c4ccccc4)nc(-c4ccc5c(ccc6oc7ccccc7c65)c4)n3)cc2)cc1]

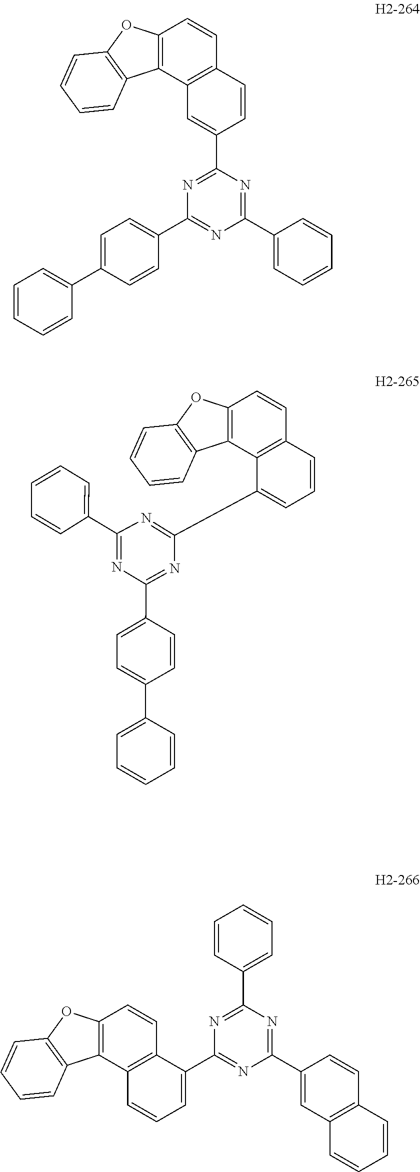 [SMILES: c1ccc(-c2ccc(-c3nc(-c4ccccc4)nc(-c4ccc5ccc6oc7ccccc7c6c5c4)n3)cc2)cc1 ; c1ccc(-c2ccc(-c3nc(-c4ccccc4)nc(-c4cccc5ccc6oc7ccccc7c6c45)n3)cc2)cc1 ; c1ccc(-c2nc(-c3ccc4ccccc4c3)nc(-c3cccc4c3ccc3oc5ccccc5c34)n2)cc1]

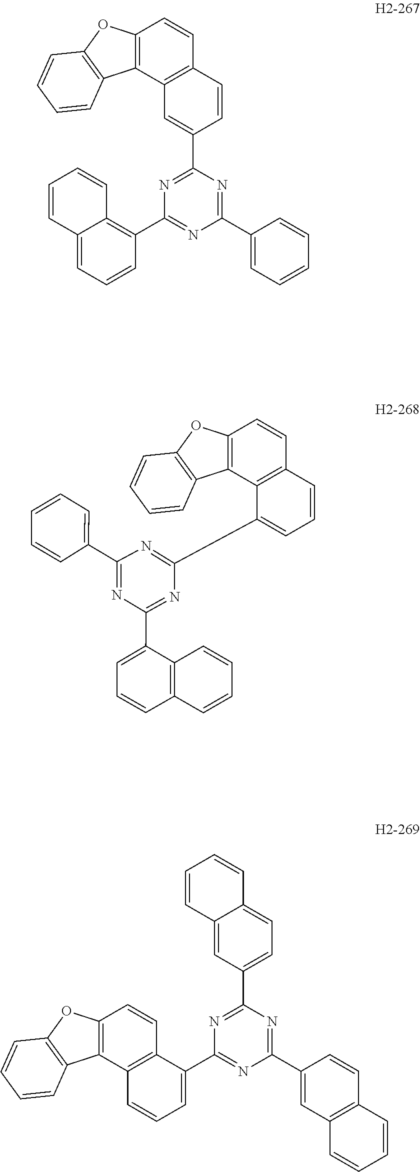 [SMILES: c1ccc(-c2nc(-c3ccc4ccc5oc6ccccc6c5c4c3)nc(-c3cccc4ccccc34)n2)cc1 ; c1ccc(-c2nc(-c3cccc4ccccc34)nc(-c3cccc4ccc5oc6ccccc6c5c34)n2)cc1 ; c1ccc2cc(-c3nc(-c4ccc5ccccc5c4)nc(-c4cccc5c4ccc4oc6ccccc6c45)n3)ccc2c1]

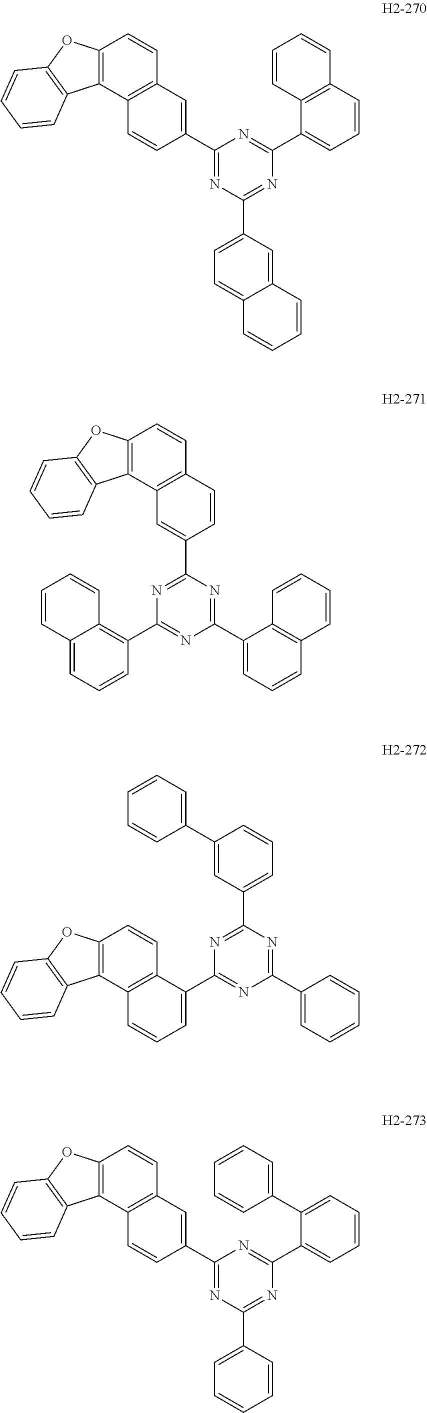 [SMILES: c1ccc2cc(-c3nc(-c4ccc5c(ccc6oc7ccccc7c65)c4)nc(-c4cccc5ccccc45)n3)ccc2c1 ; c1ccc2c(-c3nc(-c4ccc5ccc6oc7ccccc7c6c5c4)nc(-c4cccc5ccccc45)n3)cccc2c1 ; c1ccc(-c2cccc(-c3nc(-c4ccccc4)nc(-c4cccc5c4ccc4oc6ccccc6c45)n3)c2)cc1 ; c1ccc(-c2nc(-c3ccc4c(ccc5oc6ccccc6c54)c3)nc(-c3ccccc3-c3ccccc3)n2)cc1]

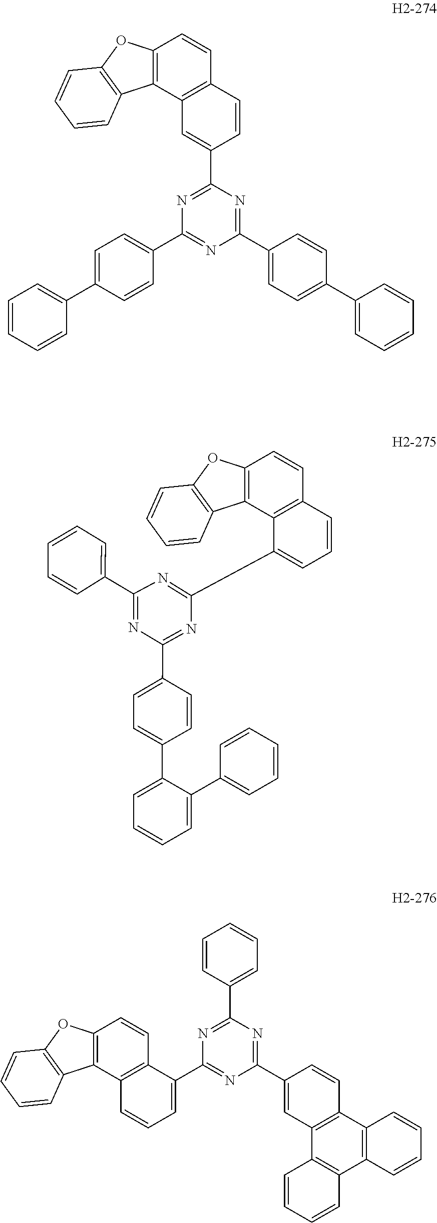 [SMILES: c1ccc(-c2ccc(-c3nc(-c4ccc(-c5ccccc5)cc4)nc(-c4ccc5ccc6oc7ccccc7c6c5c4)n3)cc2)cc1 ; c1ccc(-c2nc(-c3ccc(-c4ccccc4-c4ccccc4)cc3)nc(-c3cccc4ccc5oc6ccccc6c5c34)n2)cc1 ; c1ccc(-c2nc(-c3ccc4c5ccccc5c5ccccc5c4c3)nc(-c3cccc4c3ccc3oc5ccccc5c34)n2)cc1]

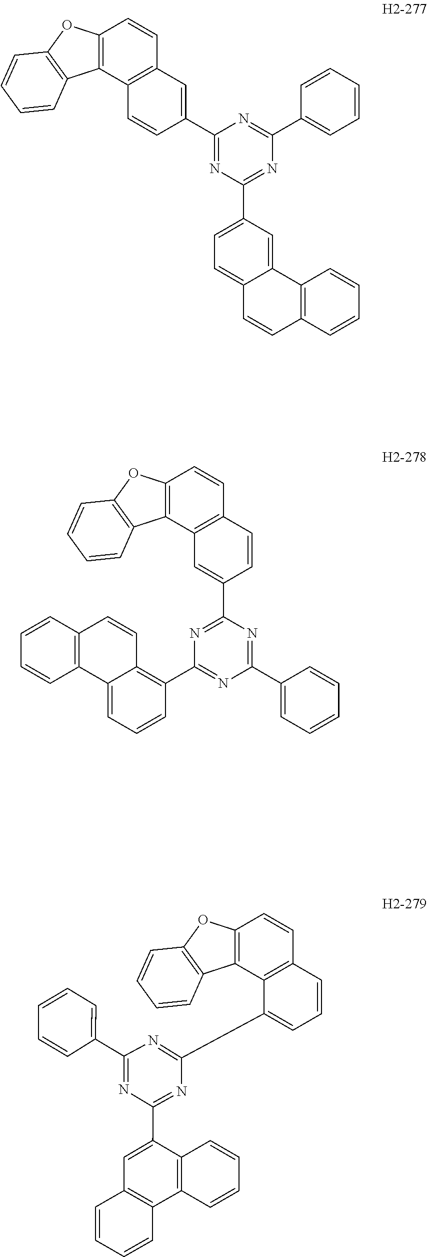 [SMILES: c1ccc(-c2nc(-c3ccc4c(ccc5oc6ccccc6c54)c3)nc(-c3ccc4ccc5ccccc5c4c3)n2)cc1 ; c1ccc(-c2nc(-c3ccc4ccc5oc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5oc6ccccc6c5c34)n2)cc1]

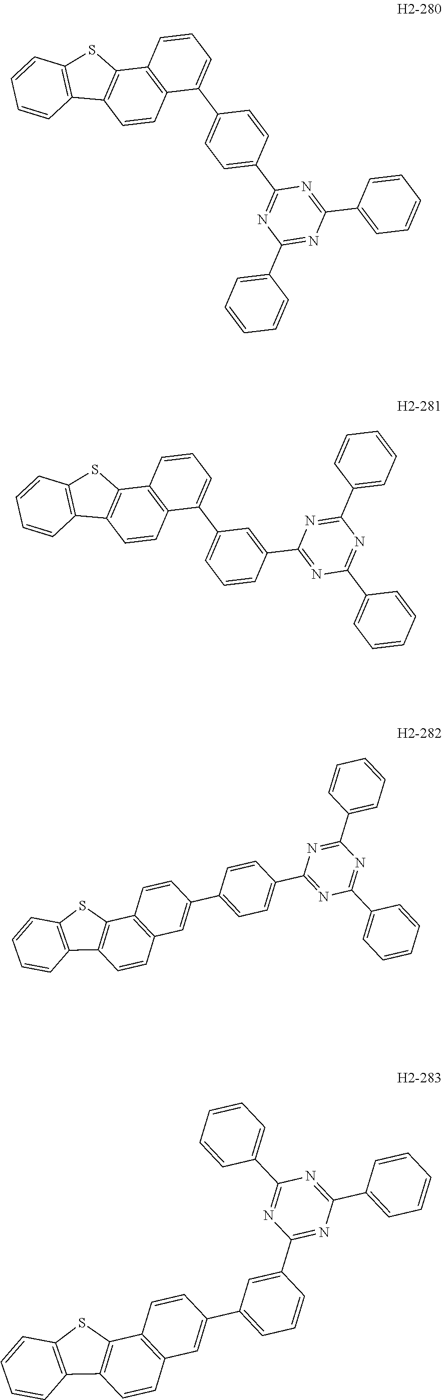 [SMILES: c1ccc(-c2nc(-c3ccccc3)nc(-c3ccc(-c4cccc5c4ccc4c6ccccc6sc54)cc3)n2)cc1 ; c1ccc(-c2nc(-c3ccccc3)nc(-c3cccc(-c4cccc5c4ccc4c6ccccc6sc54)c3)n2)cc1 ; c1ccc(-c2nc(-c3ccccc3)nc(-c3ccc(-c4ccc5c(ccc6c7ccccc7sc56)c4)cc3)n2)cc1 ; c1ccc(-c2nc(-c3ccccc3)nc(-c3cccc(-c4ccc5c(ccc6c7ccccc7sc56)c4)c3)n2)cc1]

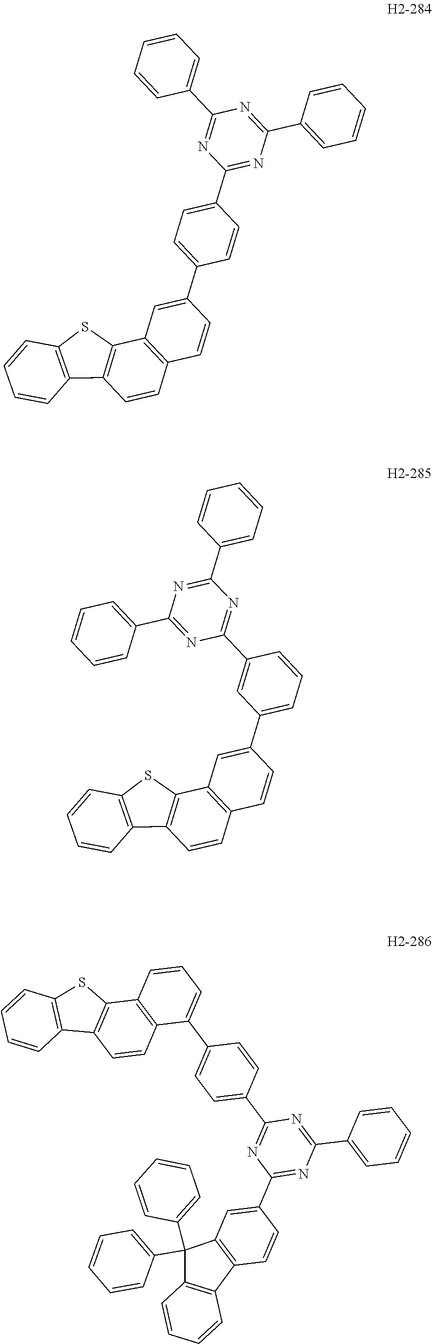 [SMILES: c1ccc(-c2nc(-c3ccccc3)nc(-c3ccc(-c4ccc5ccc6c7ccccc7sc6c5c4)cc3)n2)cc1 ; c1ccc(-c2nc(-c3ccccc3)nc(-c3cccc(-c4ccc5ccc6c7ccccc7sc6c5c4)c3)n2)cc1 ; c1ccc(-c2nc(-c3ccc(-c4cccc5c4ccc4c6ccccc6sc54)cc3)nc(-c3ccc4c(c3)C(c3ccccc3)(c3ccccc3)c3ccccc3-4)n2)cc1]

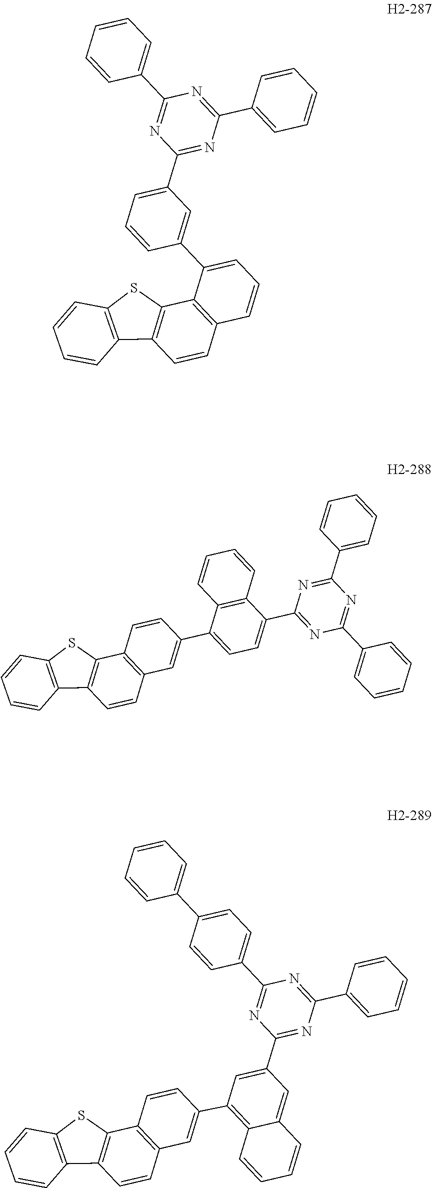 [SMILES: c1ccc(-c2nc(-c3ccccc3)nc(-c3cccc(-c4cccc5ccc6c7ccccc7sc6c45)c3)n2)cc1 ; c1ccc(-c2nc(-c3ccccc3)nc(-c3ccc(-c4ccc5c(ccc6c7ccccc7sc56)c4)c4ccccc34)n2)cc1 ; c1ccc(-c2ccc(-c3nc(-c4ccccc4)nc(-c4cc(-c5ccc6c(ccc7c8ccccc8sc67)c5)c5ccccc5c4)n3)cc2)cc1]

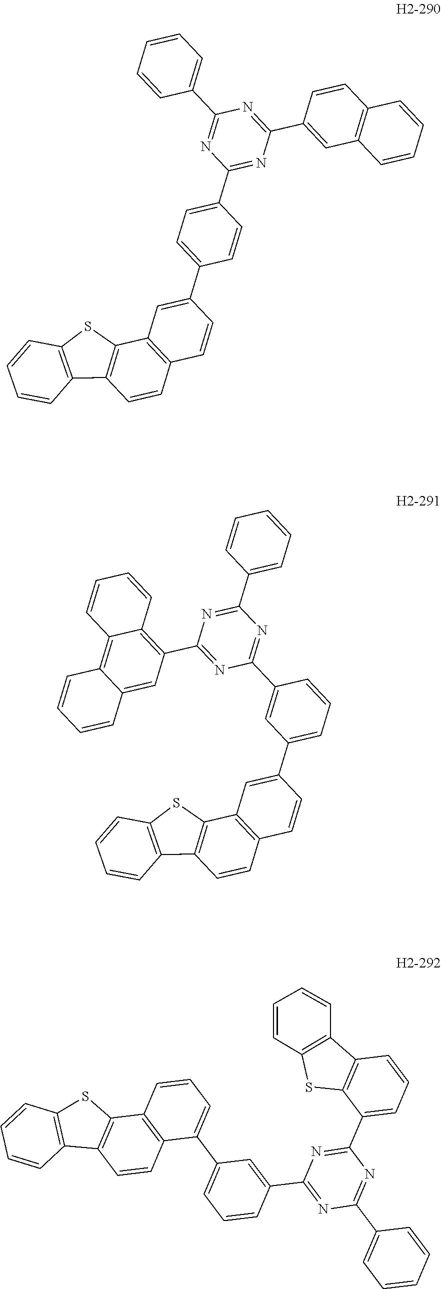 [SMILES: c1ccc(-c2nc(-c3ccc(-c4ccc5ccc6c7ccccc7sc6c5c4)cc3)nc(-c3ccc4ccccc4c3)n2)cc1 ; c1ccc(-c2nc(-c3cccc(-c4ccc5ccc6c7ccccc7sc6c5c4)c3)nc(-c3cc4ccccc4c4ccccc34)n2)cc1 ; c1ccc(-c2nc(-c3cccc(-c4cccc5c4ccc4c6ccccc6sc54)c3)nc(-c3cccc4c3sc3ccccc34)n2)cc1]

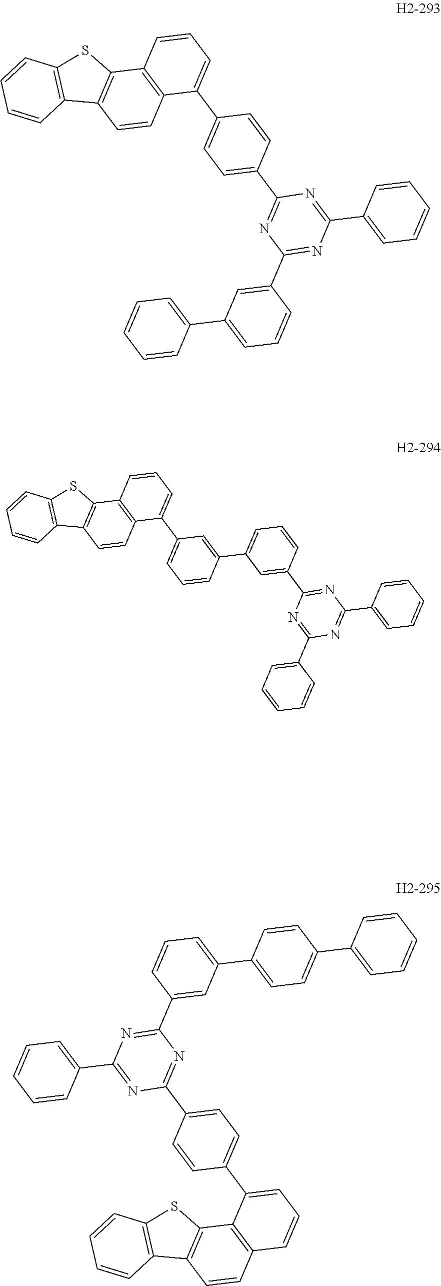 [SMILES: c1ccc(-c2cccc(-c3nc(-c4ccccc4)nc(-c4ccc(-c5cccc6c5ccc5c7ccccc7sc65)cc4)n3)c2)cc1 ; c1ccc(-c2nc(-c3ccccc3)nc(-c3cccc(-c4cccc(-c5cccc6c5ccc5c7ccccc7sc65)c4)c3)n2)cc1 ; c1ccc(-c2ccc(-c3cccc(-c4nc(-c5ccccc5)nc(-c5ccc(-c6cccc7ccc8c9ccccc9sc8c67)cc5)n4)c3)cc2)cc1]

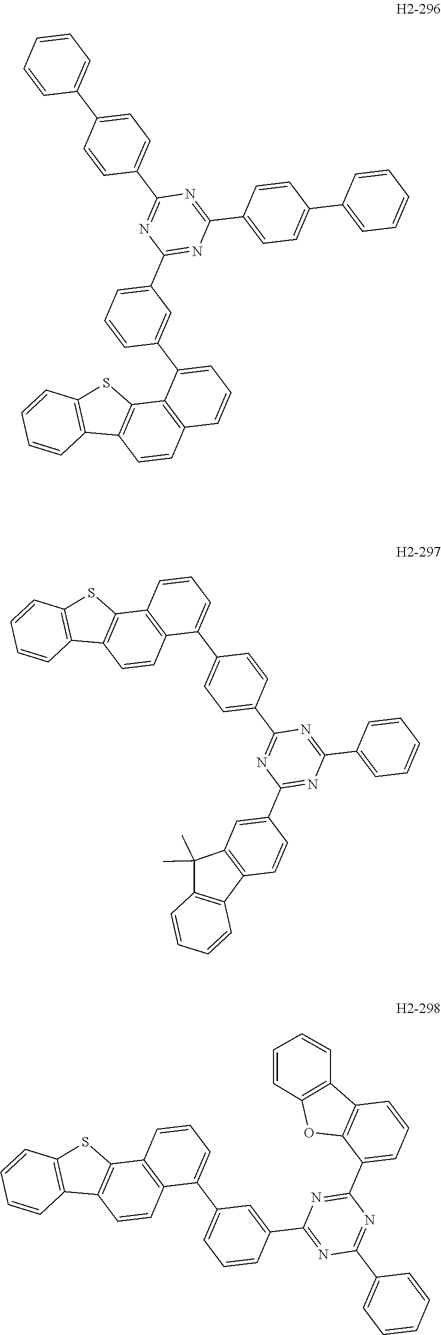 [SMILES: c1ccc(-c2ccc(-c3nc(-c4ccc(-c5ccccc5)cc4)nc(-c4cccc(-c5cccc6ccc7c8ccccc8sc7c56)c4)n3)cc2)cc1 ; CC1(C)c2ccccc2-c2ccc(-c3nc(-c4ccccc4)nc(-c4ccc(-c5cccc6c5ccc5c7ccccc7sc65)cc4)n3)cc21 ; c1ccc(-c2nc(-c3cccc(-c4cccc5c4ccc4c6ccccc6sc54)c3)nc(-c3cccc4c3oc3ccccc34)n2)cc1]

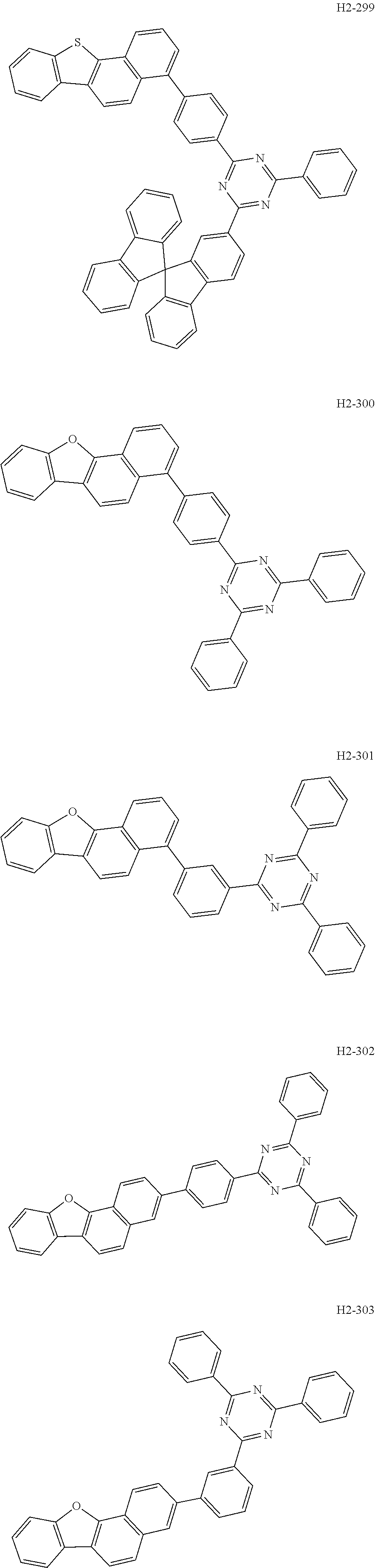 [SMILES: c1ccc(-c2nc(-c3ccc(-c4cccc5c4ccc4c6ccccc6sc54)cc3)nc(-c3ccc4c(c3)C3(c5ccccc5-c5ccccc53)c3ccccc3-4)n2)cc1 ; c1ccc(-c2nc(-c3ccccc3)nc(-c3ccc(-c4cccc5c4ccc4c6ccccc6oc54)cc3)n2)cc1 ; c1ccc(-c2nc(-c3ccccc3)nc(-c3cccc(-c4cccc5c4ccc4c6ccccc6oc54)c3)n2)cc1 ; c1ccc(-c2nc(-c3ccccc3)nc(-c3ccc(-c4ccc5c(ccc6c7ccccc7oc56)c4)cc3)n2)cc1 ; c1ccc(-c2nc(-c3ccccc3)nc(-c3cccc(-c4ccc5c(ccc6c7ccccc7oc56)c4)c3)n2)cc1]

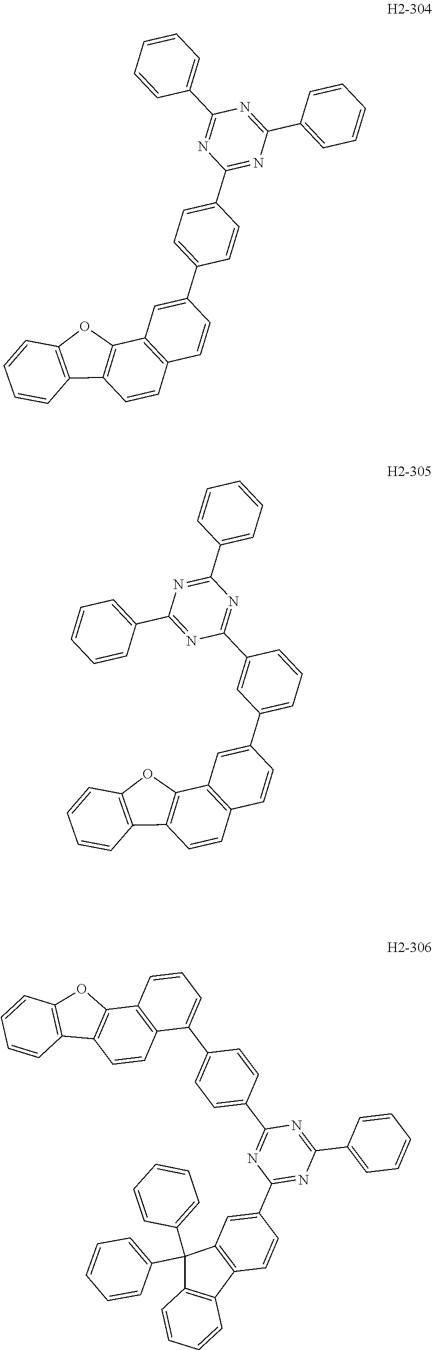 [SMILES: c1ccc(-c2nc(-c3ccccc3)nc(-c3ccc(-c4ccc5ccc6c7ccccc7oc6c5c4)cc3)n2)cc1 ; c1ccc(-c2nc(-c3ccccc3)nc(-c3cccc(-c4ccc5ccc6c7ccccc7oc6c5c4)c3)n2)cc1 ; c1ccc(-c2nc(-c3ccc(-c4cccc5c4ccc4c6ccccc6oc54)cc3)nc(-c3ccc4c(c3)C(c3ccccc3)(c3ccccc3)c3ccccc3-4)n2)cc1]

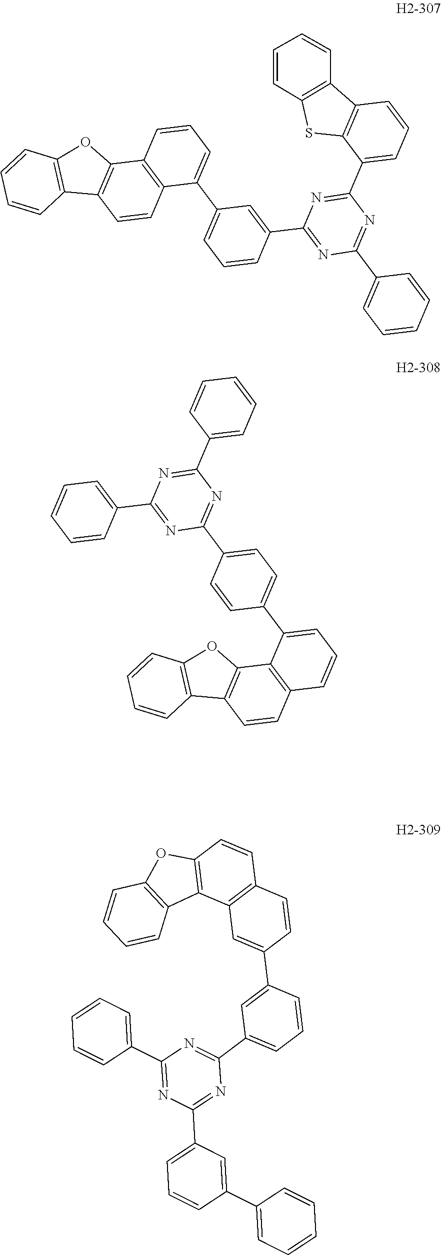 [SMILES: c1ccc(-c2nc(-c3cccc(-c4cccc5c4ccc4c6ccccc6oc54)c3)nc(-c3cccc4c3sc3ccccc34)n2)cc1 ; c1ccc(-c2nc(-c3ccccc3)nc(-c3ccc(-c4cccc5ccc6c7ccccc7oc6c45)cc3)n2)cc1 ; c1ccc(-c2cccc(-c3nc(-c4ccccc4)nc(-c4cccc(-c5ccc6ccc7oc8ccccc8c7c6c5)c4)n3)c2)cc1]

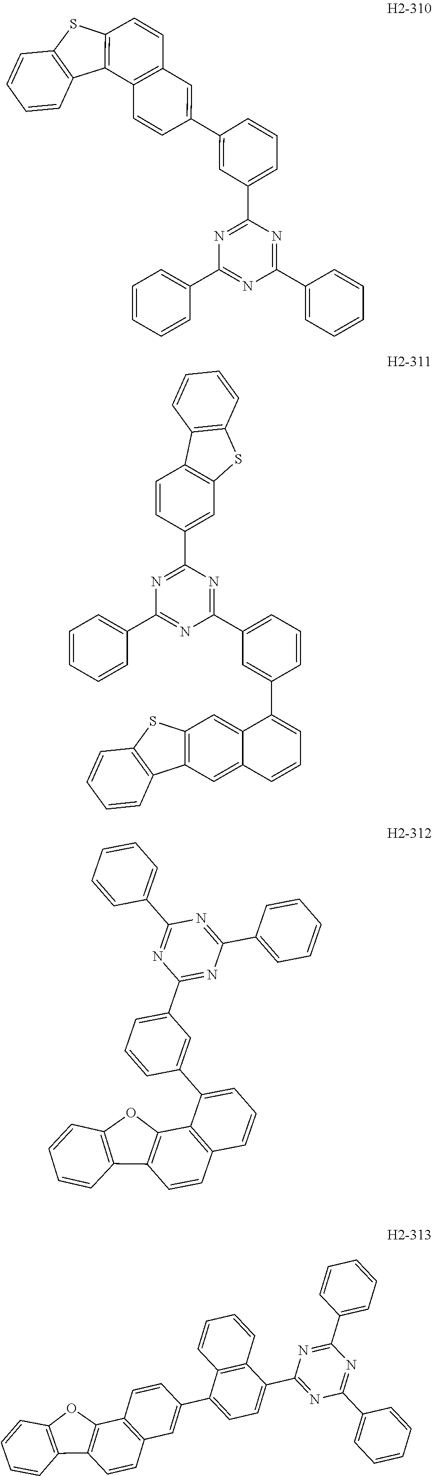

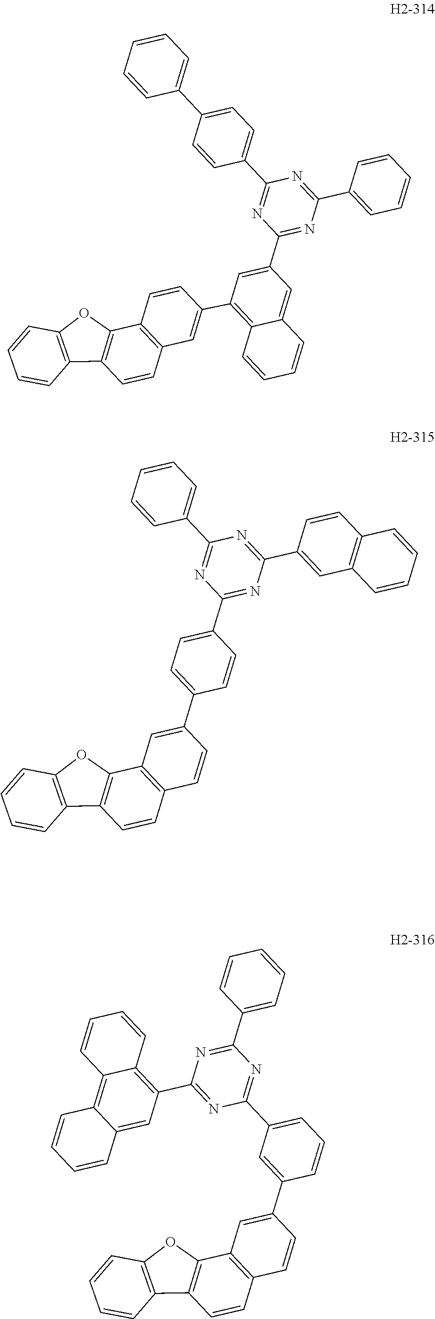 [SMILES: c1ccc(-c2ccc(-c3nc(-c4ccccc4)nc(-c4cc(-c5ccc6c(ccc7c8ccccc8oc67)c5)c5ccccc5c4)n3)cc2)cc1 ; c1ccc(-c2nc(-c3ccc(-c4ccc5ccc6c7ccccc7oc6c5c4)cc3)nc(-c3ccc4ccccc4c3)n2)cc1 ; c1ccc(-c2nc(-c3cccc(-c4ccc5ccc6c7ccccc7oc6c5c4)c3)nc(-c3cc4ccccc4c4ccccc34)n2)cc1]

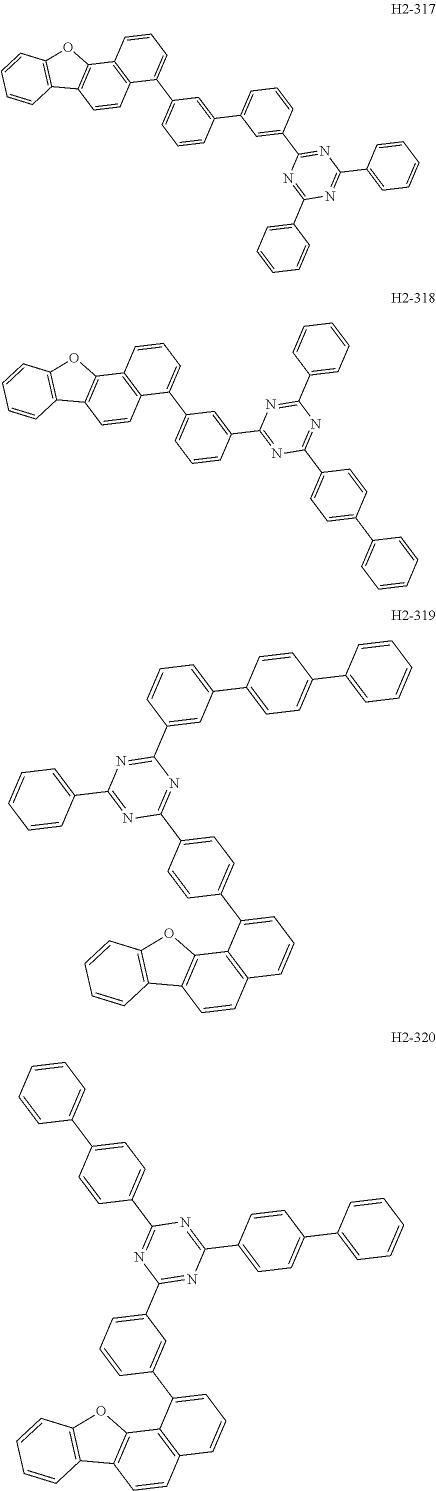 [SMILES: c1ccc(-c2nc(-c3ccccc3)nc(-c3cccc(-c4cccc(-c5cccc6c5ccc5c7ccccc7oc65)c4)c3)n2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2ccc(-c3cccc(-c4nc(-c5ccccc5)nc(-c5ccc(-c6cccc7ccc8c9ccccc9oc8c67)cc5)n4)c3)cc2)cc1 ; c1ccc(-c2ccc(-c3nc(-c4ccc(-c5ccccc5)cc4)nc(-c4cccc(-c5cccc6ccc7c8ccccc8oc7c56)c4)n3)cc2)cc1]

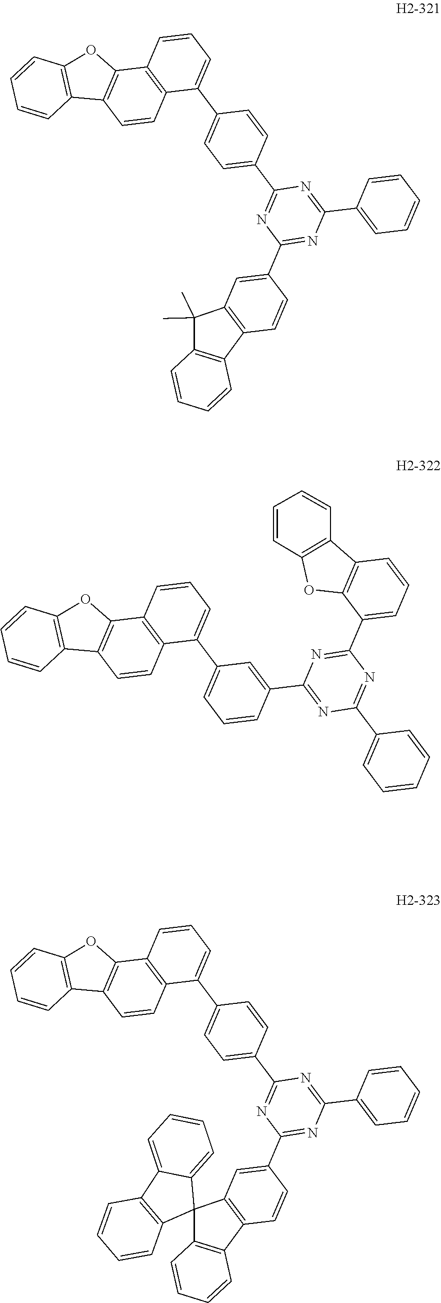 [SMILES: CC1(C)c2ccccc2-c2ccc(-c3nc(-c4ccccc4)nc(-c4ccc(-c5cccc6c5ccc5c7ccccc7oc65)cc4)n3)cc21 ; c1ccc(-c2nc(-c3cccc(-c4cccc5c4ccc4c6ccccc6oc54)c3)nc(-c3cccc4c3oc3ccccc34)n2)cc1 ; c1ccc(-c2nc(-c3ccc(-c4cccc5c4ccc4c6ccccc6oc54)cc3)nc(-c3ccc4c(c3)C3(c5ccccc5-c5ccccc53)c3ccccc3-4)n2)cc1]

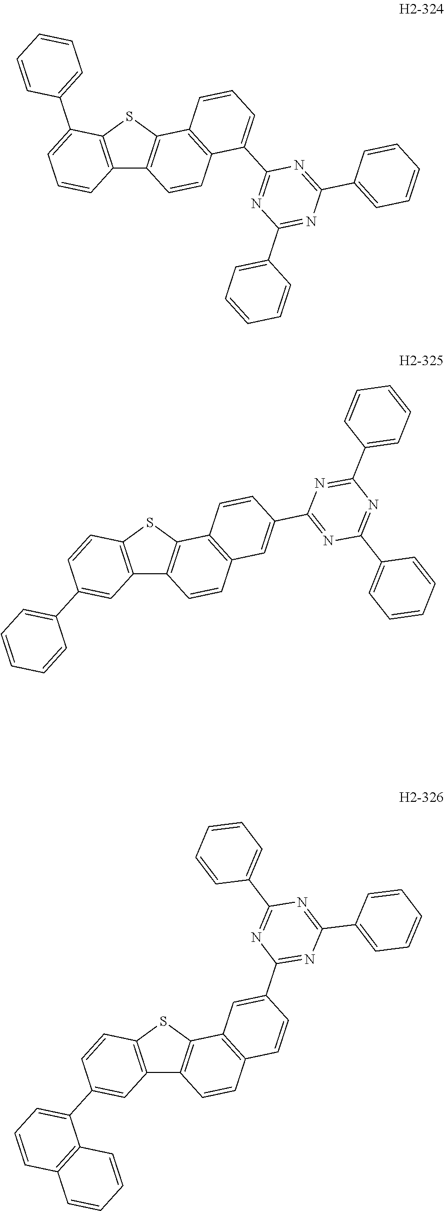 [SMILES: c1ccc(-c2nc(-c3ccccc3)nc(-c3cccc4c3ccc3c5cccc(-c6ccccc6)c5sc43)n2)cc1 ; c1ccc(-c2ccc3sc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6sc5c4c3)n2)cc1]

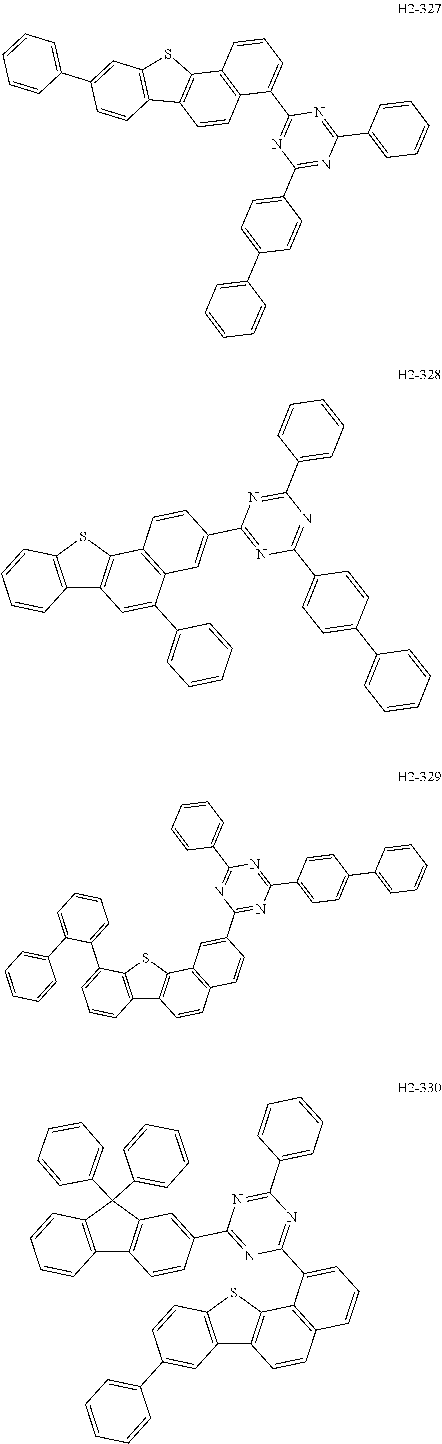 [SMILES: c1ccc(-c2ccc(-c3nc(-c4ccccc4)nc(-c4cccc5c4ccc4c6ccc(-c7ccccc7)cc6sc54)n3)cc2)cc1 ; c1ccc(-c2ccc(-c3nc(-c4ccccc4)nc(-c4ccc5c(c4)c(-c4ccccc4)cc4c6ccccc6sc54)n3)cc2)cc1 ; c1ccc(-c2ccc(-c3nc(-c4ccccc4)nc(-c4ccc5ccc6c7cccc(-c8ccccc8-c8ccccc8)c7sc6c5c4)n3)cc2)cc1 ; c1ccc(-c2ccc3sc4c(ccc5cccc(-c6nc(-c7ccccc7)nc(-c7ccc8c(c7)C(c7ccccc7)(c7ccccc7)c7ccccc7-8)n6)c54)c3c2)cc1]

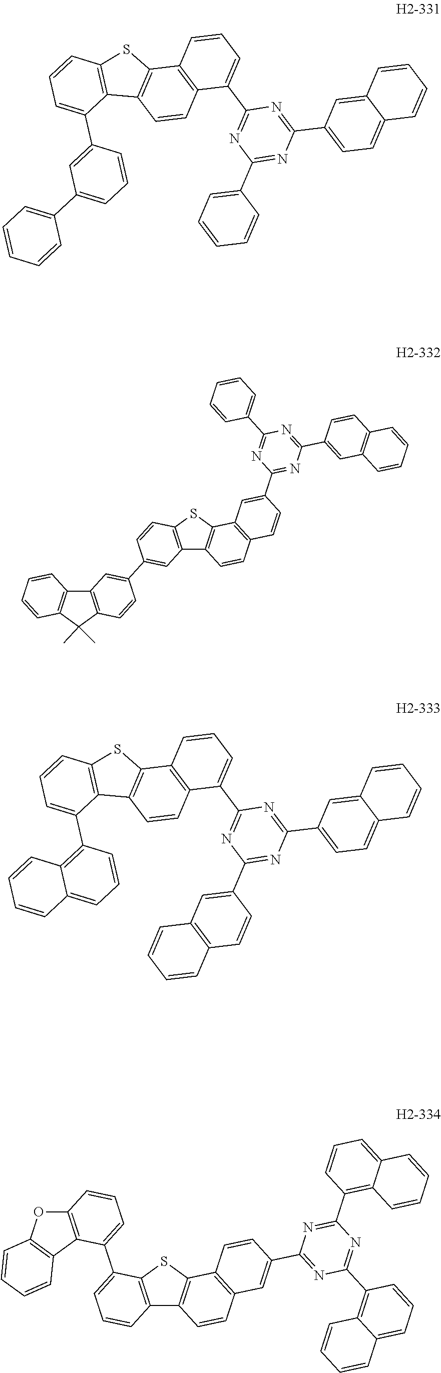 [SMILES: c1ccc(-c2cccc(-c3cccc4sc5c6cccc(-c7nc(-c8ccccc8)nc(-c8ccc9ccccc9c8)n7)c6ccc5c34)c2)cc1 ; CC1(C)c2ccccc2-c2cc(-c3ccc4sc5c6cc(-c7nc(-c8ccccc8)nc(-c8ccc9ccccc9c8)n7)ccc6ccc5c4c3)ccc21 ; c1ccc2cc(-c3nc(-c4ccc5ccccc5c4)nc(-c4cccc5c4ccc4c5sc5cccc(-c6cccc7ccccc67)c54)n3)ccc2c1 ; c1ccc2c(-c3nc(-c4ccc5c(ccc6c7cccc(-c8cccc9oc%10ccccc%10c89)c7sc56)c4)nc(-c4cccc5ccccc45)n3)cccc2c1]

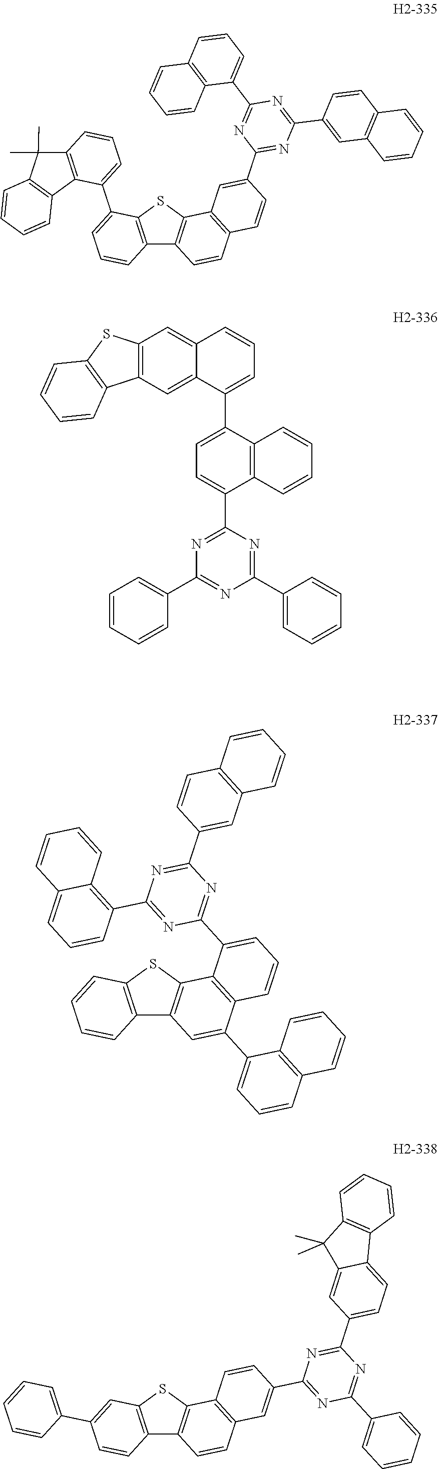 [SMILES: CC1(C)c2ccccc2-c2c(-c3cccc4c3sc3c5cc(-c6nc(-c7ccc8ccccc8c7)nc(-c7cccc8ccccc78)n6)ccc5ccc43)cccc21 ; c1ccc(-c2nc(-c3ccccc3)nc(-c3ccc(-c4cccc5cc6sc7ccccc7c6cc45)c4ccccc34)n2)cc1 ; c1ccc2cc(-c3nc(-c4cccc5ccccc45)nc(-c4cccc5c(-c6cccc7ccccc67)cc6c7ccccc7sc6c45)n3)ccc2c1 ; CC1(C)c2ccccc2-c2ccc(-c3nc(-c4ccccc4)nc(-c4ccc5c(ccc6c7ccc(-c8ccccc8)cc7sc56)c4)n3)cc21]

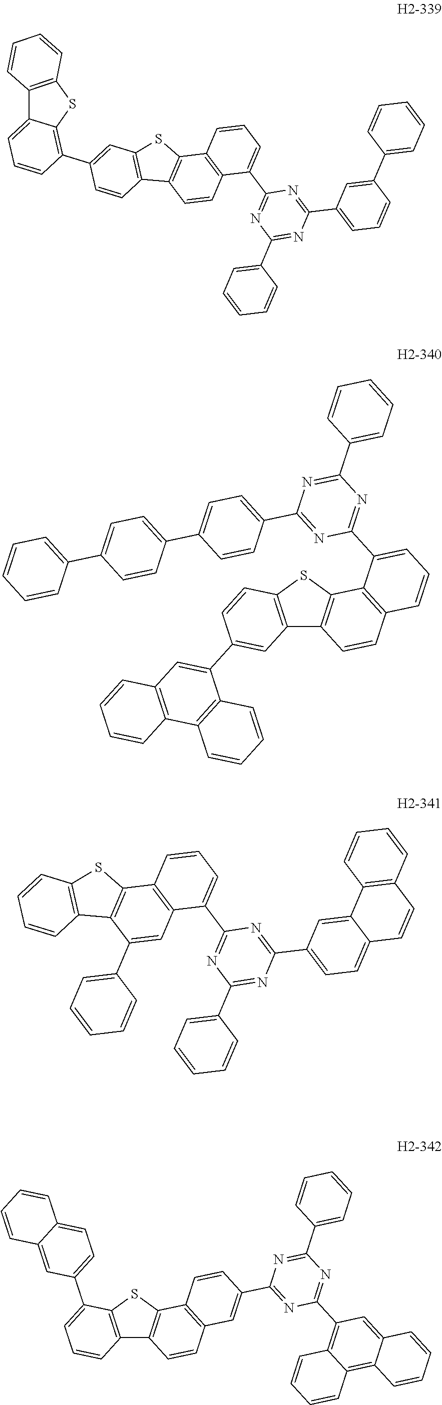 [SMILES: c1ccc(-c2cccc(-c3nc(-c4ccccc4)nc(-c4cccc5c4ccc4c6ccc(-c7cccc8c7sc7ccccc78)cc6sc54)n3)c2)cc1 ; c1ccc(-c2ccc(-c3ccc(-c4nc(-c5ccccc5)nc(-c5cccc6ccc7c8cc(-c9cc%10ccccc%10c%10ccccc9%10)ccc8sc7c56)n4)cc3)cc2)cc1 ; c1ccc(-c2nc(-c3ccc4ccc5ccccc5c4c3)nc(-c3cccc4c3cc(-c3ccccc3)c3c5ccccc5sc43)n2)cc1 ; c1ccc(-c2nc(-c3ccc4c(ccc5c6cccc(-c7ccc8ccccc8c7)c6sc45)c3)nc(-c3cc4ccccc4c4ccccc34)n2)cc1]

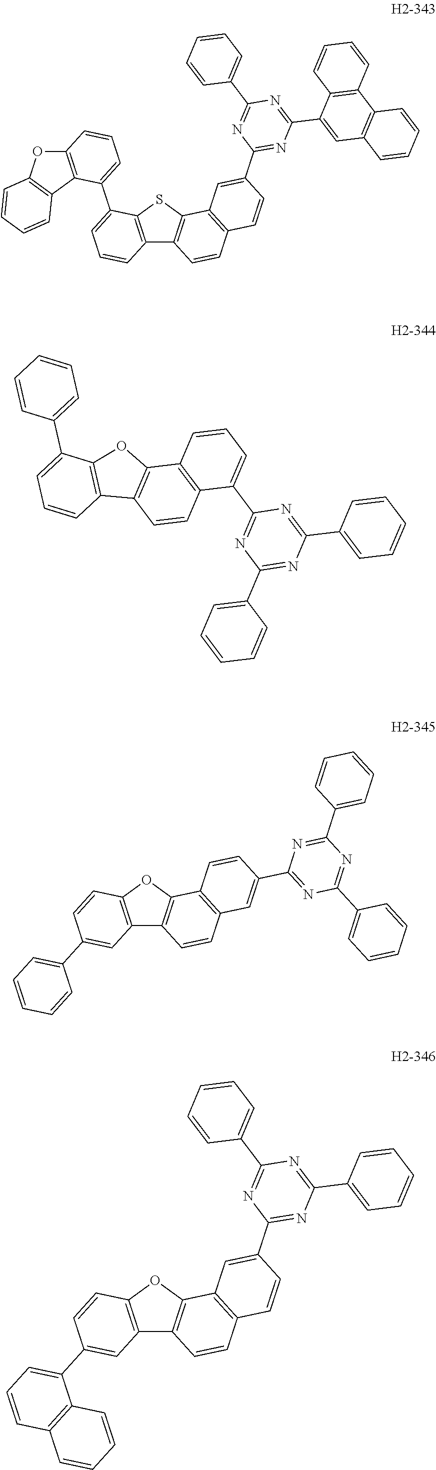 [SMILES: c1ccc(-c2nc(-c3ccc4ccc5c6cccc(-c7cccc8oc9ccccc9c78)c6sc5c4c3)nc(-c3cc4ccccc4c4ccccc34)n2)cc1 ; c1ccc(-c2nc(-c3ccccc3)nc(-c3cccc4c3ccc3c5cccc(-c6ccccc6)c5oc43)n2)cc1 ; c1ccc(-c2ccc3oc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6oc5c4c3)n2)cc1]

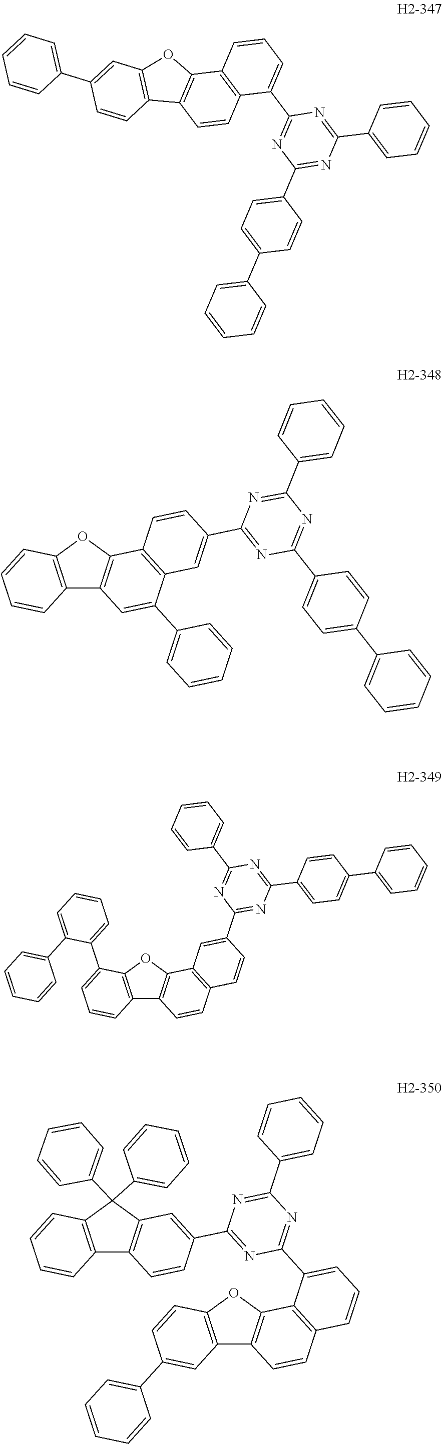 [SMILES: c1ccc(-c2ccc(-c3nc(-c4ccccc4)nc(-c4cccc5c4ccc4c6ccc(-c7ccccc7)cc6oc54)n3)cc2)cc1 ; c1ccc(-c2ccc(-c3nc(-c4ccccc4)nc(-c4ccc5c(c4)c(-c4ccccc4)cc4c6ccccc6oc54)n3)cc2)cc1 ; c1ccc(-c2ccc(-c3nc(-c4ccccc4)nc(-c4ccc5ccc6c7cccc(-c8ccccc8-c8ccccc8)c7oc6c5c4)n3)cc2)cc1 ; c1ccc(-c2ccc3oc4c(ccc5cccc(-c6nc(-c7ccccc7)nc(-c7ccc8c(c7)C(c7ccccc7)(c7ccccc7)c7ccccc7-8)n6)c54)c3c2)cc1]

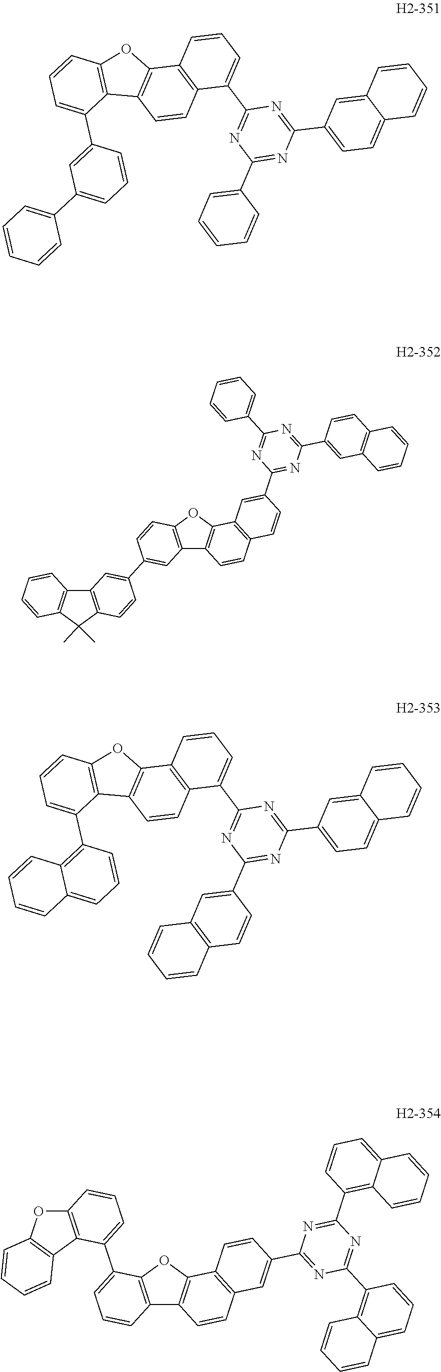 [SMILES: c1ccc(-c2cccc(-c3cccc4oc5c6cccc(-c7nc(-c8ccccc8)nc(-c8ccc9ccccc9c8)n7)c6ccc5c34)c2)cc1 ; CC1(C)c2ccccc2-c2cc(-c3ccc4oc5c6cc(-c7nc(-c8ccccc8)nc(-c8ccc9ccccc9c8)n7)ccc6ccc5c4c3)ccc21 ; c1ccc2cc(-c3nc(-c4ccc5ccccc5c4)nc(-c4cccc5c4ccc4c5oc5cccc(-c6cccc7ccccc67)c54)n3)ccc2c1 ; c1ccc2c(-c3nc(-c4ccc5c(ccc6c7cccc(-c8cccc9oc%10ccccc%10c89)c7oc56)c4)nc(-c4cccc5ccccc45)n3)cccc2c1]

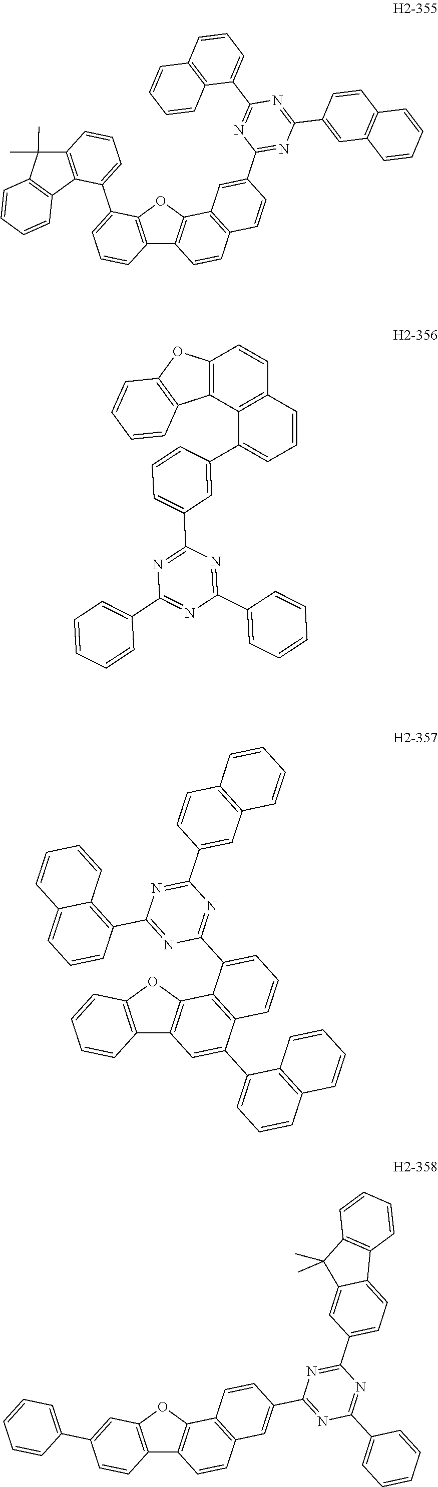 [SMILES: CC1(C)c2ccccc2-c2c(-c3cccc4c3oc3c5cc(-c6nc(-c7ccc8ccccc8c7)nc(-c7cccc8ccccc78)n6)ccc5ccc43)cccc21 ; c1ccc(-c2nc(-c3ccccc3)nc(-c3cccc(-c4cccc5ccc6oc7ccccc7c6c45)c3)n2)cc1 ; c1ccc2cc(-c3nc(-c4cccc5ccccc45)nc(-c4cccc5c(-c6cccc7ccccc67)cc6c7ccccc7oc6c45)n3)ccc2c1 ; CC1(C)c2ccccc2-c2ccc(-c3nc(-c4ccccc4)nc(-c4ccc5c(ccc6c7ccc(-c8ccccc8)cc7oc56)c4)n3)cc21]

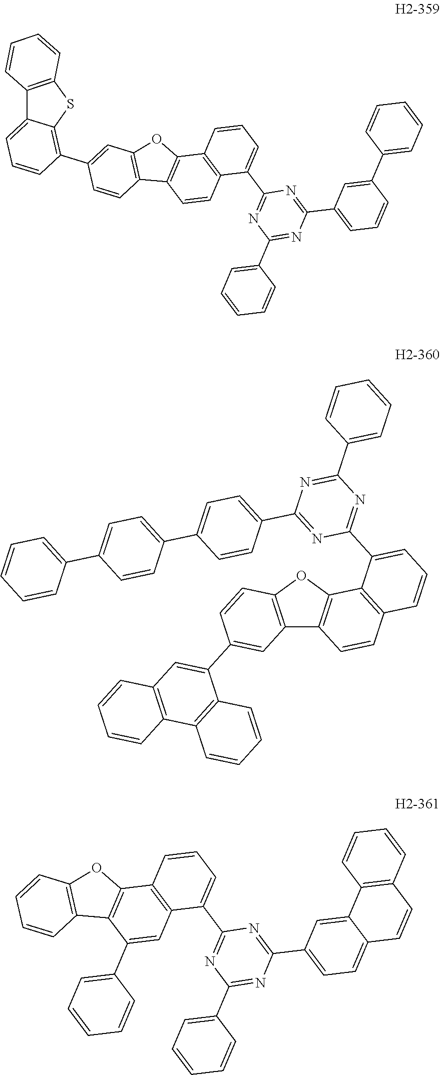 [SMILES: c1ccc(-c2cccc(-c3nc(-c4ccccc4)nc(-c4cccc5c4ccc4c6ccc(-c7cccc8c7sc7ccccc78)cc6oc54)n3)c2)cc1 ; c1ccc(-c2ccc(-c3ccc(-c4nc(-c5ccccc5)nc(-c5cccc6ccc7c8cc(-c9cc%10ccccc%10c%10ccccc9%10)ccc8oc7c56)n4)cc3)cc2)cc1 ; c1ccc(-c2nc(-c3ccc4ccc5ccccc5c4c3)nc(-c3cccc4c3cc(-c3ccccc3)c3c5ccccc5oc43)n2)cc1]

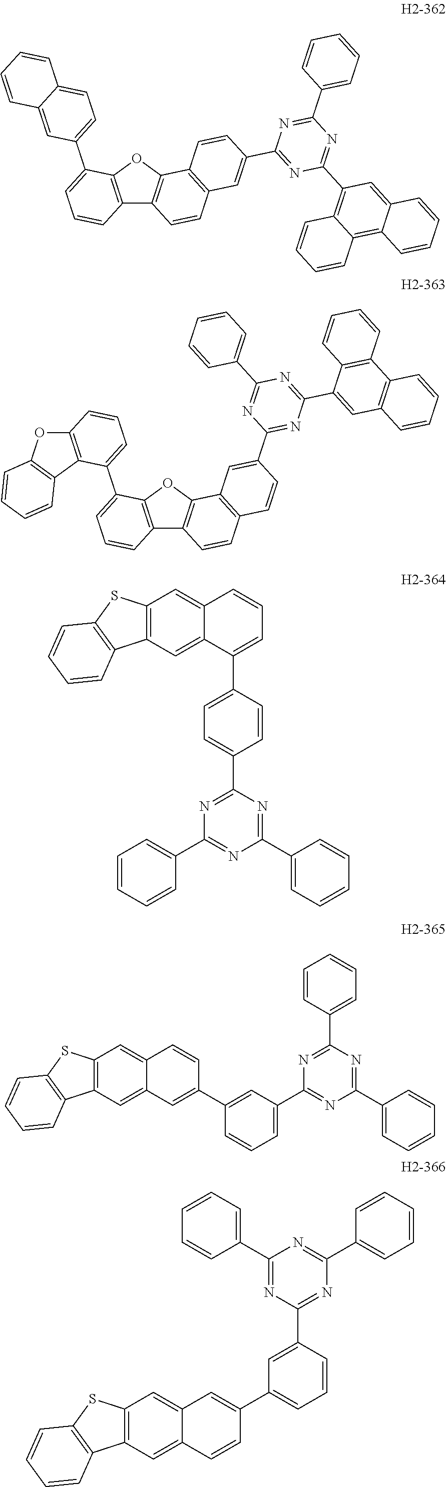 [SMILES: c1ccc(-c2nc(-c3ccc4c(ccc5c6cccc(-c7ccc8ccccc8c7)c6oc45)c3)nc(-c3cc4ccccc4c4ccccc34)n2)cc1 ; c1ccc(-c2nc(-c3ccc4ccc5c6cccc(-c7cccc8oc9ccccc9c78)c6oc5c4c3)nc(-c3cc4ccccc4c4ccccc34)n2)cc1 ; c1ccc(-c2nc(-c3ccccc3)nc(-c3ccc(-c4cccc5cc6sc7ccccc7c6cc45)cc3)n2)cc1 ; c1ccc(-c2nc(-c3ccccc3)nc(-c3cccc(-c4ccc5cc6sc7ccccc7c6cc5c4)c3)n2)cc1 ; c1ccc(-c2nc(-c3ccccc3)nc(-c3cccc(-c4ccc5cc6c(cc5c4)sc4ccccc46)c3)n2)cc1]

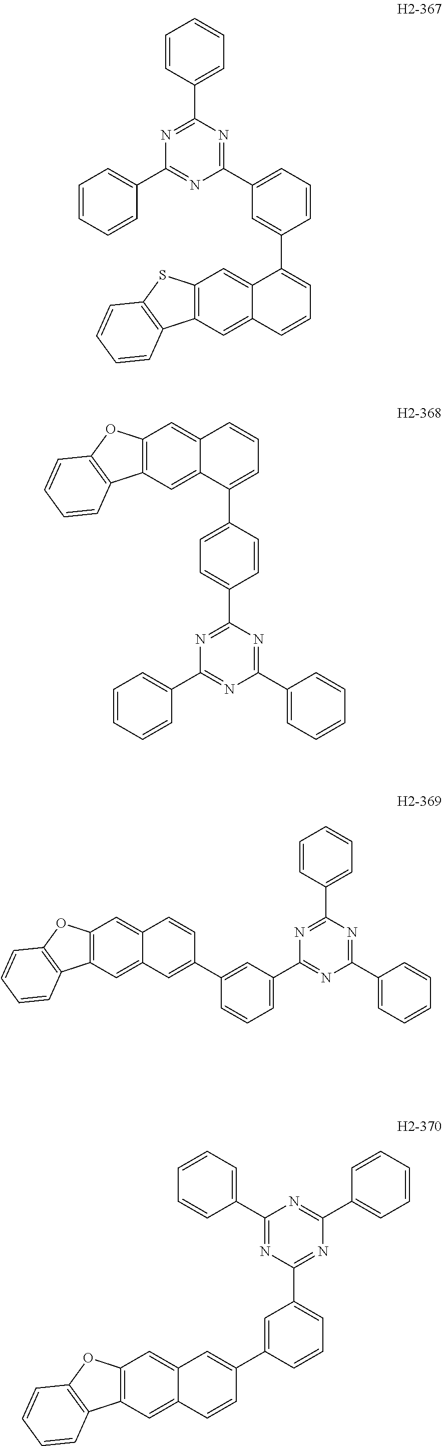 [SMILES: c1ccc(-c2nc(-c3ccccc3)nc(-c3cccc(-c4cccc5cc6c(cc45)sc4ccccc46)c3)n2)cc1 ; c1ccc(-c2nc(-c3ccccc3)nc(-c3ccc(-c4cccc5cc6oc7ccccc7c6cc45)cc3)n2)cc1 ; c1ccc(-c2nc(-c3ccccc3)nc(-c3cccc(-c4ccc5cc6oc7ccccc7c6cc5c4)c3)n2)cc1 ; c1ccc(-c2nc(-c3ccccc3)nc(-c3cccc(-c4ccc5cc6c(cc5c4)oc4ccccc46)c3)n2)cc1]

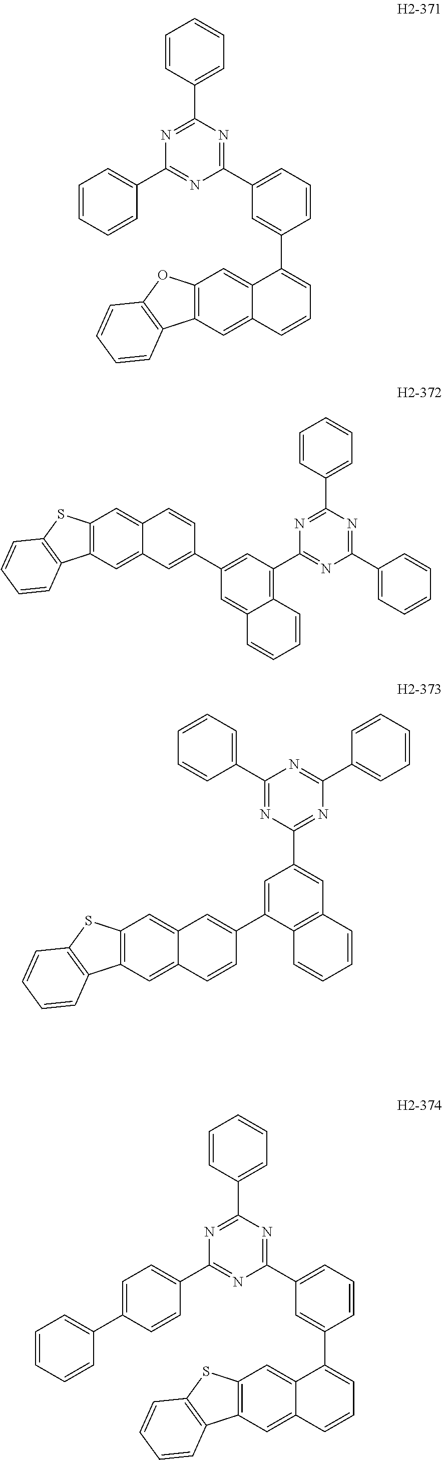 [SMILES: c1ccc(-c2nc(-c3ccccc3)nc(-c3cccc(-c4cccc5cc6c(cc45)oc4ccccc46)c3)n2)cc1 ; c1ccc(-c2nc(-c3ccccc3)nc(-c3cc(-c4ccc5cc6sc7ccccc7c6cc5c4)cc4ccccc34)n2)cc1 ; c1ccc(-c2nc(-c3ccccc3)nc(-c3cc(-c4ccc5cc6c(cc5c4)sc4ccccc46)c4ccccc4c3)n2)cc1 ; c1ccc(-c2ccc(-c3nc(-c4ccccc4)nc(-c4cccc(-c5cccc6cc7c(cc56)sc5ccccc57)c4)n3)cc2)cc1]

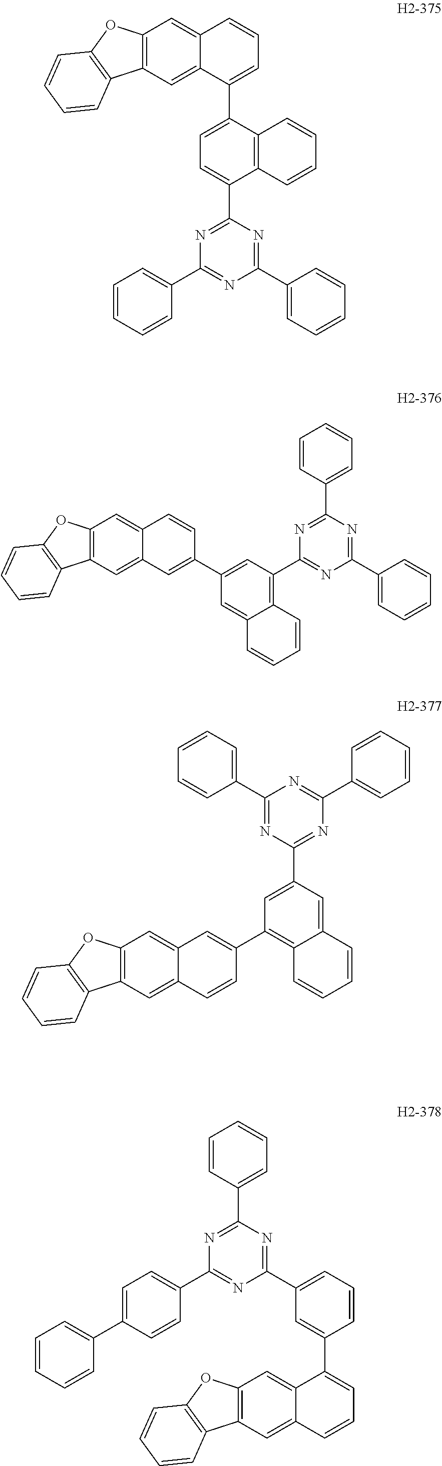 [SMILES: c1ccc(-c2nc(-c3ccccc3)nc(-c3ccc(-c4cccc5cc6oc7ccccc7c6cc45)c4ccccc34)n2)cc1 ; c1ccc(-c2nc(-c3ccccc3)nc(-c3cc(-c4ccc5cc6oc7ccccc7c6cc5c4)cc4ccccc34)n2)cc1 ; c1ccc(-c2nc(-c3ccccc3)nc(-c3cc(-c4ccc5cc6c(cc5c4)oc4ccccc46)c4ccccc4c3)n2)cc1 ; c1ccc(-c2ccc(-c3nc(-c4ccccc4)nc(-c4cccc(-c5cccc6cc7c(cc56)oc5ccccc57)c4)n3)cc2)cc1]

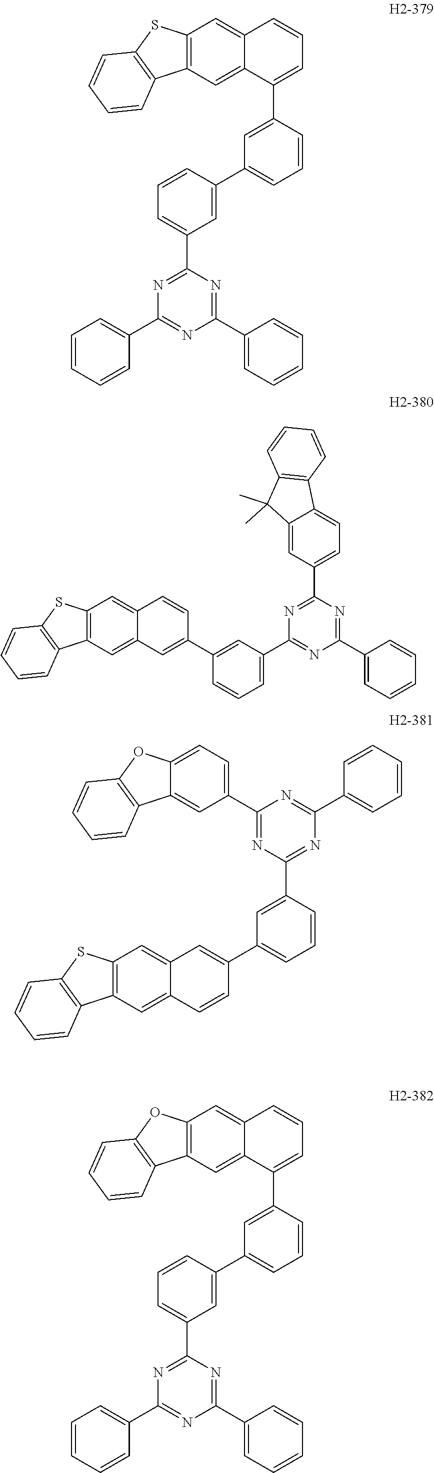

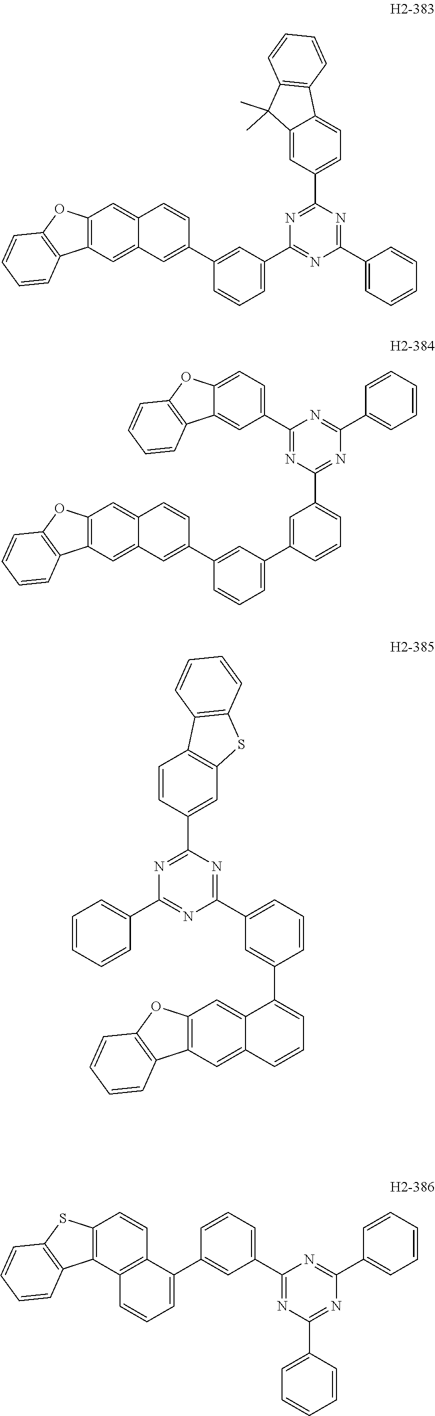 [SMILES: CC1(C)c2ccccc2-c2ccc(-c3nc(-c4ccccc4)nc(-c4cccc(-c5ccc6cc7oc8ccccc8c7cc6c5)c4)n3)cc21 ; c1ccc(-c2nc(-c3cccc(-c4cccc(-c5ccc6cc7oc8ccccc8c7cc6c5)c4)c3)nc(-c3ccc4oc5ccccc5c4c3)n2)cc1 ; c1ccc(-c2nc(-c3cccc(-c4cccc5cc6c(cc45)oc4ccccc46)c3)nc(-c3ccc4c(c3)sc3ccccc34)n2)cc1 ; c1ccc(-c2nc(-c3ccccc3)nc(-c3cccc(-c4cccc5c4ccc4sc6ccccc6c45)c3)n2)cc1]

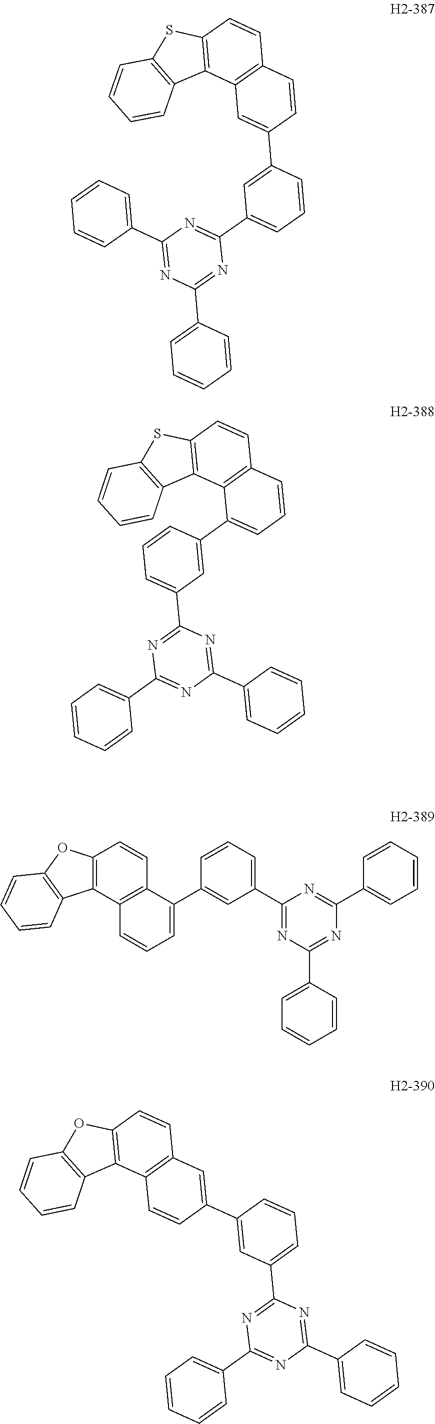

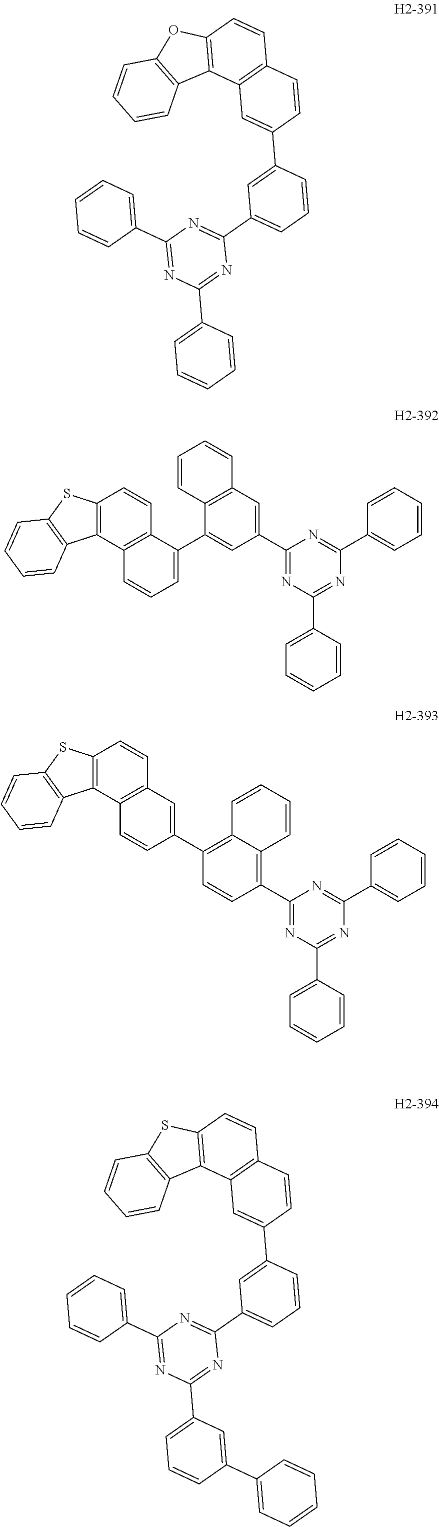 [SMILES: c1ccc(-c2nc(-c3ccccc3)nc(-c3cccc(-c4ccc5ccc6oc7ccccc7c6c5c4)c3)n2)cc1 ; c1ccc(-c2nc(-c3ccccc3)nc(-c3cc(-c4cccc5c4ccc4sc6ccccc6c45)c4ccccc4c3)n2)cc1 ; c1ccc(-c2nc(-c3ccccc3)nc(-c3ccc(-c4ccc5c(ccc6sc7ccccc7c65)c4)c4ccccc34)n2)cc1 ; c1ccc(-c2cccc(-c3nc(-c4ccccc4)nc(-c4cccc(-c5ccc6ccc7sc8ccccc8c7c6c5)c4)n3)c2)cc1]

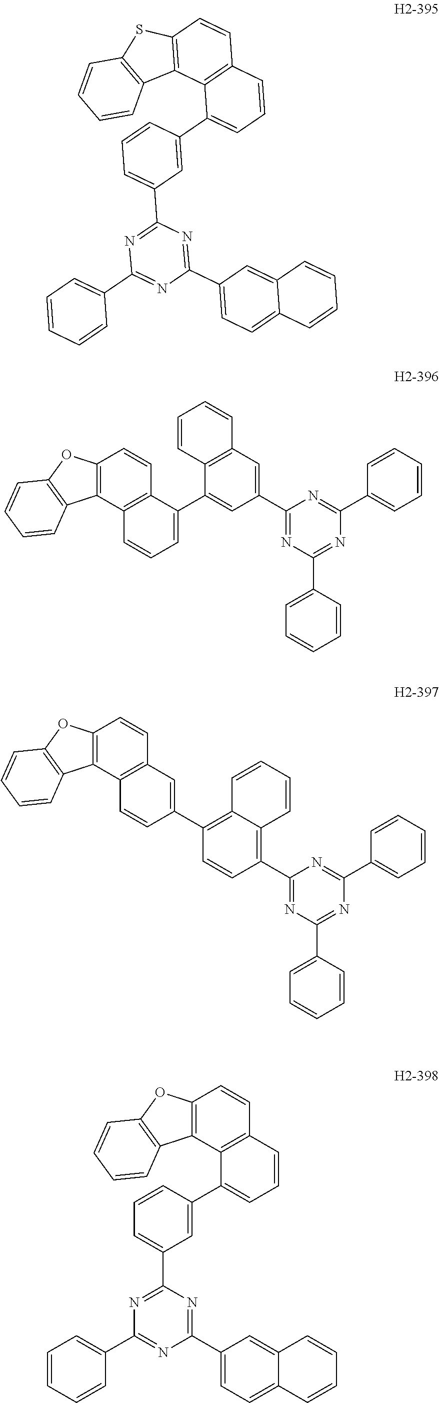 [SMILES: c1ccc(-c2nc(-c3cccc(-c4cccc5ccc6sc7ccccc7c6c45)c3)nc(-c3ccc4ccccc4c3)n2)cc1 ; c1ccc(-c2nc(-c3ccccc3)nc(-c3cc(-c4cccc5c4ccc4oc6ccccc6c45)c4ccccc4c3)n2)cc1 ; c1ccc(-c2nc(-c3ccccc3)nc(-c3ccc(-c4ccc5c(ccc6oc7ccccc7c65)c4)c4ccccc34)n2)cc1 ; c1ccc(-c2nc(-c3cccc(-c4cccc5ccc6oc7ccccc7c6c45)c3)nc(-c3ccc4ccccc4c3)n2)cc1]

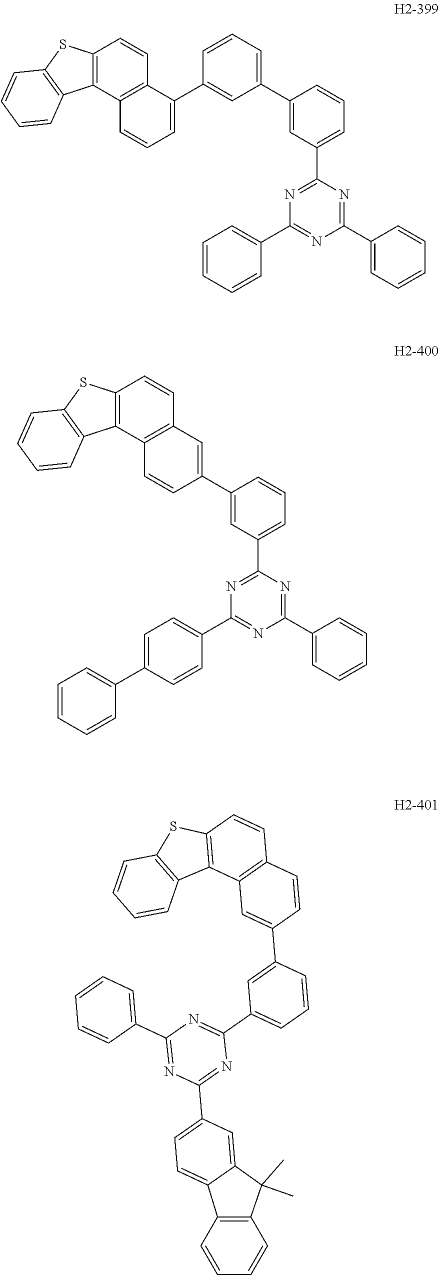 [SMILES: c1ccc(-c2nc(-c3ccccc3)nc(-c3cccc(-c4cccc(-c5cccc6c5ccc5sc7ccccc7c56)c4)c3)n2)cc1 ; c1ccc(-c2ccc(-c3nc(-c4ccccc4)nc(-c4cccc(-c5ccc6c(ccc7sc8ccccc8c76)c5)c4)n3)cc2)cc1 ; CC1(C)c2ccccc2-c2ccc(-c3nc(-c4ccccc4)nc(-c4cccc(-c5ccc6ccc7sc8ccccc8c7c6c5)c4)n3)cc21]

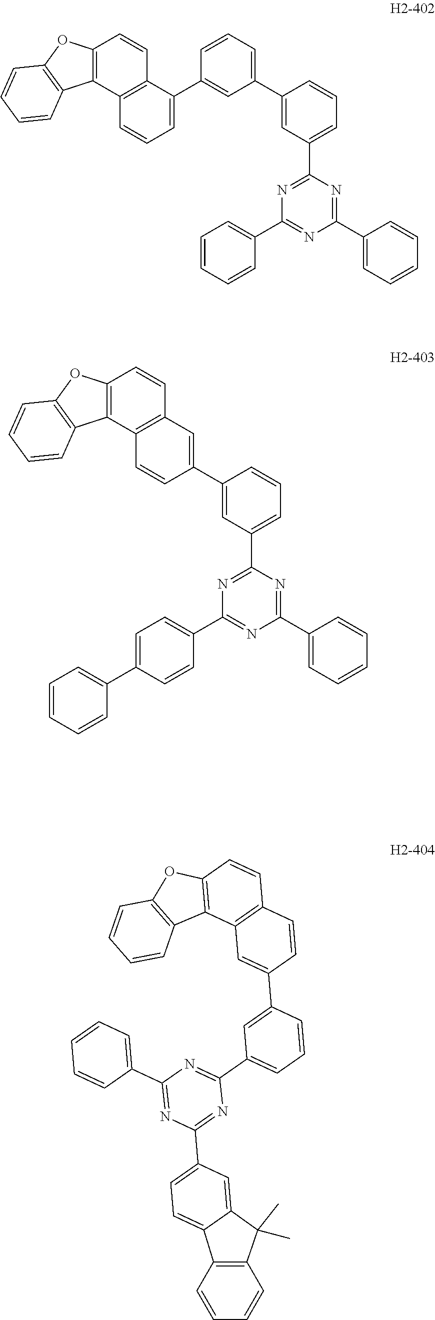 [SMILES: c1ccc(-c2nc(-c3ccccc3)nc(-c3cccc(-c4cccc(-c5cccc6c5ccc5oc7ccccc7c56)c4)c3)n2)cc1 ; c1ccc(-c2ccc(-c3nc(-c4ccccc4)nc(-c4cccc(-c5ccc6c(ccc7oc8ccccc8c76)c5)c4)n3)cc2)cc1 ; CC1(C)c2ccccc2-c2ccc(-c3nc(-c4ccccc4)nc(-c4cccc(-c5ccc6ccc7oc8ccccc8c7c6c5)c4)n3)cc21]

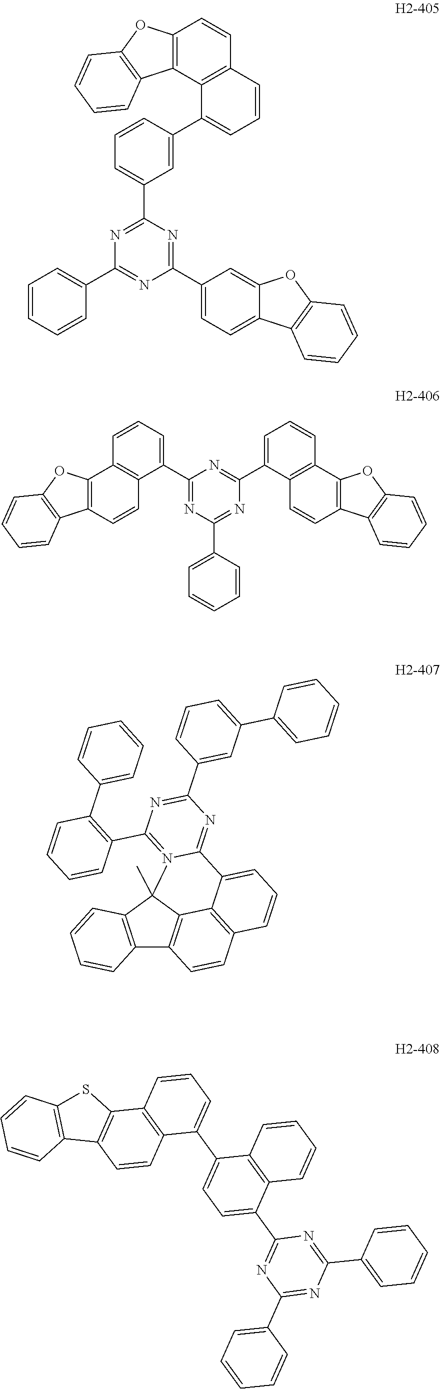 [SMILES: c1ccc(-c2nc(-c3cccc(-c4cccc5ccc6oc7ccccc7c6c45)c3)nc(-c3ccc4c(c3)oc3ccccc34)n2)cc1 ; c1ccc(-c2nc(-c3cccc4c3ccc3c5ccccc5oc43)nc(-c3cccc4c3ccc3c5ccccc5oc43)n2)cc1 ; CC1(C)c2ccccc2-c2ccc3cccc(-c4nc(-c5cccc(-c6ccccc6)c5)nc(-c5ccccc5-c5ccccc5)n4)c3c21 ; c1ccc(-c2nc(-c3ccccc3)nc(-c3ccc(-c4cccc5c4ccc4c6ccccc6sc54)c4ccccc34)n2)cc1]

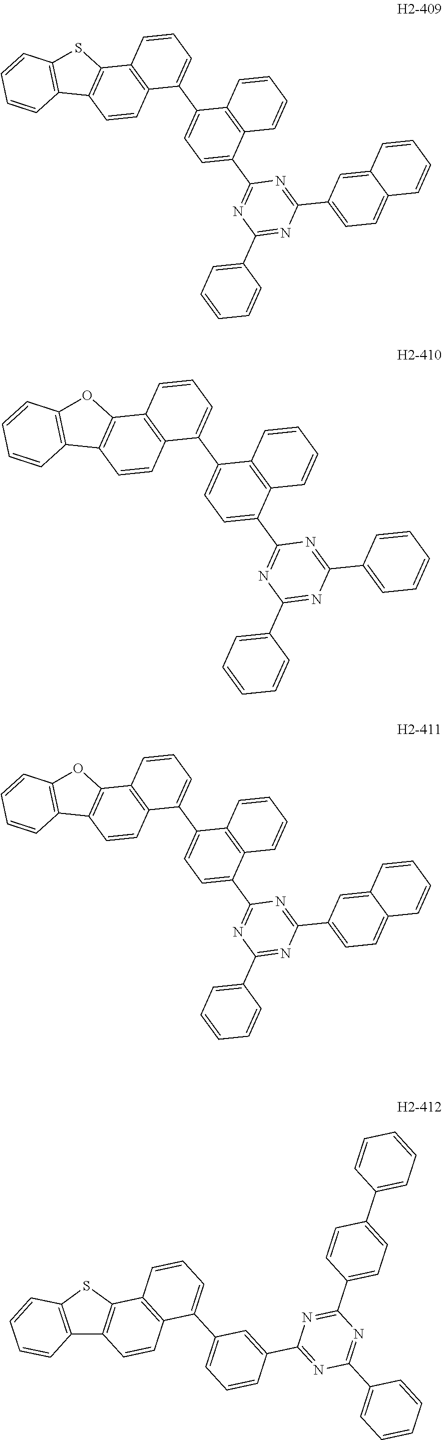 [SMILES: c1ccc(-c2nc(-c3ccc4ccccc4c3)nc(-c3ccc(-c4cccc5c4ccc4c6ccccc6sc54)c4ccccc34)n2)cc1 ; c1ccc(-c2nc(-c3ccccc3)nc(-c3ccc(-c4cccc5c4ccc4c6ccccc6oc54)c4ccccc34)n2)cc1 ; c1ccc(-c2nc(-c3ccc4ccccc4c3)nc(-c3ccc(-c4cccc5c4ccc4c6ccccc6oc54)c4ccccc34)n2)cc1 ; c1ccc(-c2ccc(-c3nc(-c4ccccc4)nc(-c4cccc(-c5cccc6c5ccc5c7ccccc7sc65)c4)n3)cc2)cc1]

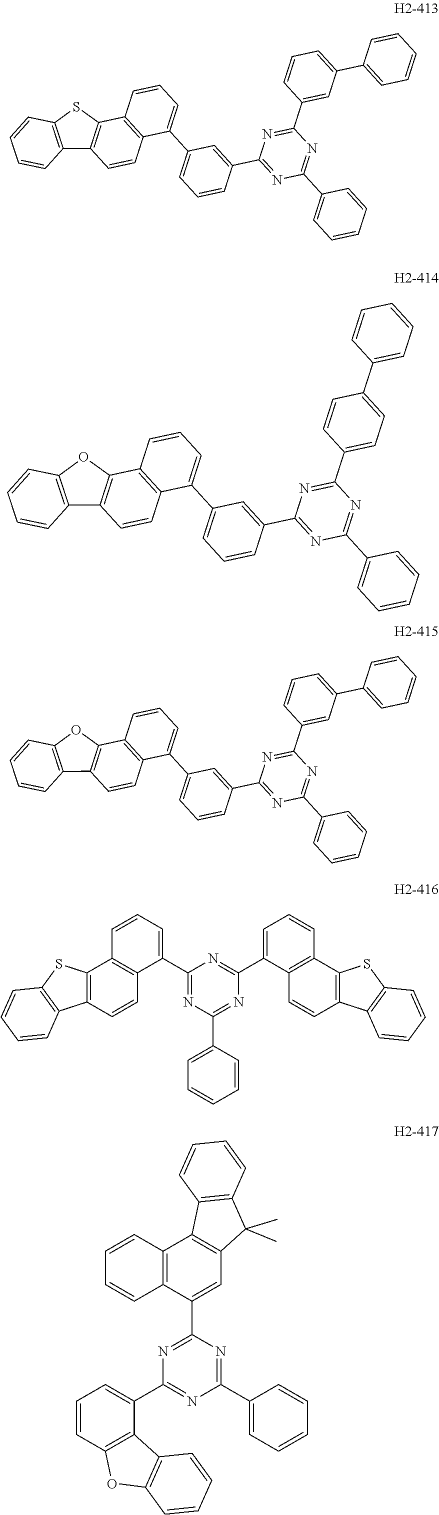

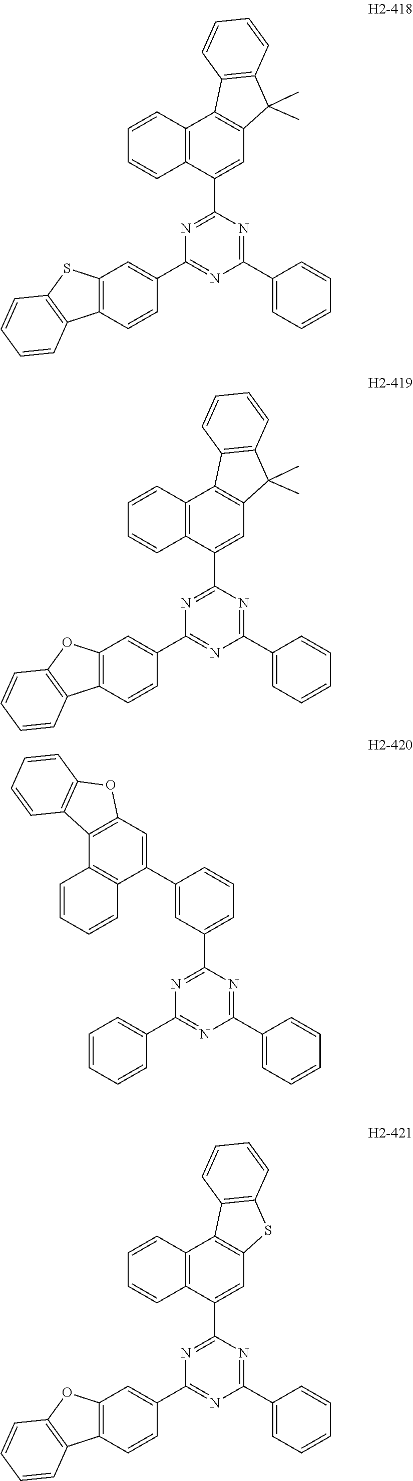

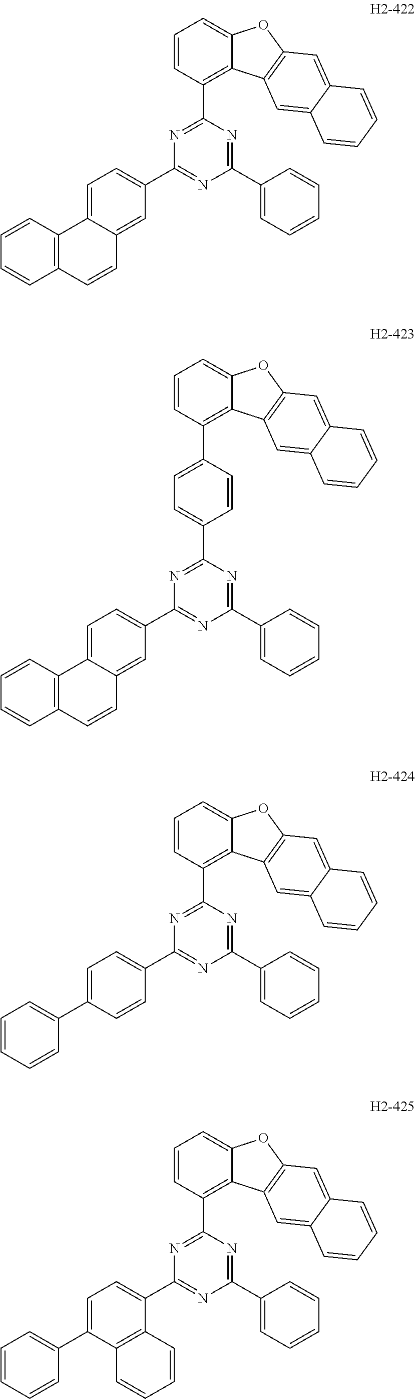 [SMILES: c1ccc(-c2nc(-c3ccc4c(ccc5ccccc54)c3)nc(-c3cccc4oc5cc6ccccc6cc5c34)n2)cc1 ; c1ccc(-c2nc(-c3ccc(-c4cccc5oc6cc7ccccc7cc6c45)cc3)nc(-c3ccc4c(ccc5ccccc54)c3)n2)cc1 ; c1ccc(-c2ccc(-c3nc(-c4ccccc4)nc(-c4cccc5oc6cc7ccccc7cc6c45)n3)cc2)cc1 ; c1ccc(-c2nc(-c3ccc(-c4ccccc4)c4ccccc34)nc(-c3cccc4oc5cc6ccccc6cc5c34)n2)cc1]

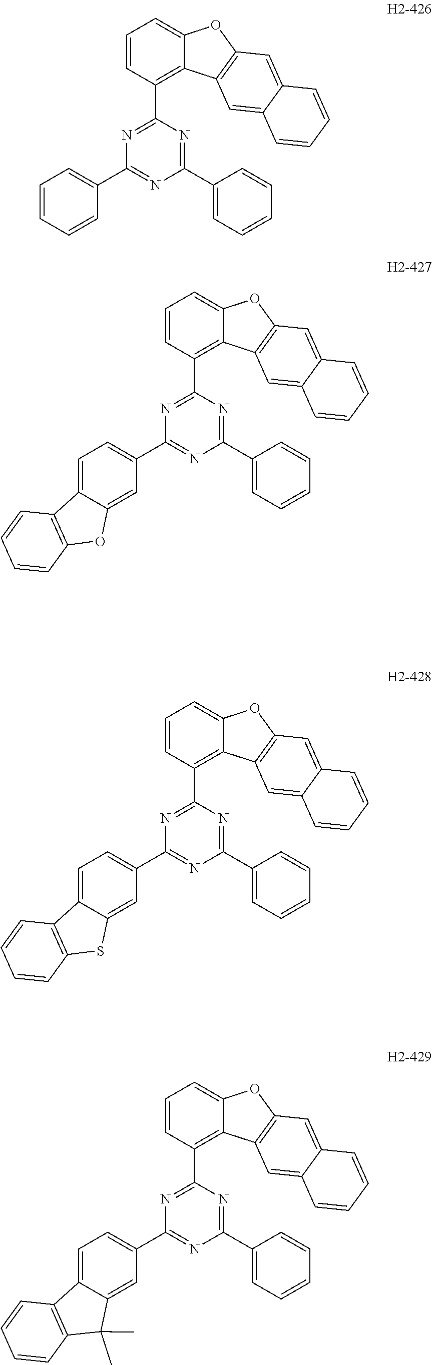 [SMILES: c1ccc(-c2nc(-c3ccccc3)nc(-c3cccc4oc5cc6ccccc6cc5c34)n2)cc1 ; c1ccc(-c2nc(-c3ccc4c(c3)oc3ccccc34)nc(-c3cccc4oc5cc6ccccc6cc5c34)n2)cc1 ; c1ccc(-c2nc(-c3ccc4c(c3)sc3ccccc34)nc(-c3cccc4oc5cc6ccccc6cc5c34)n2)cc1 ; CC1(C)c2ccccc2-c2ccc(-c3nc(-c4ccccc4)nc(-c4cccc5oc6cc7ccccc7cc6c45)n3)cc21]

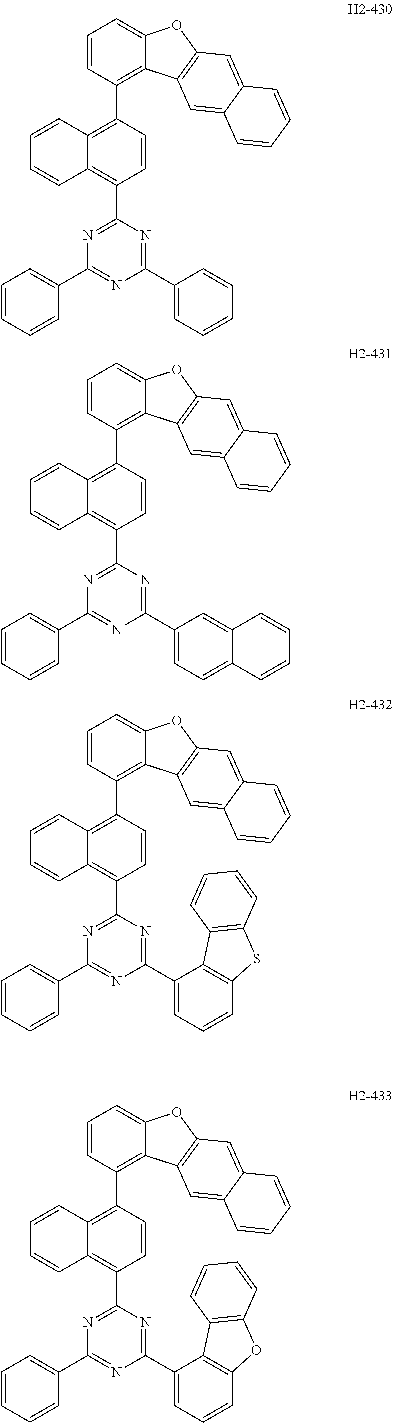

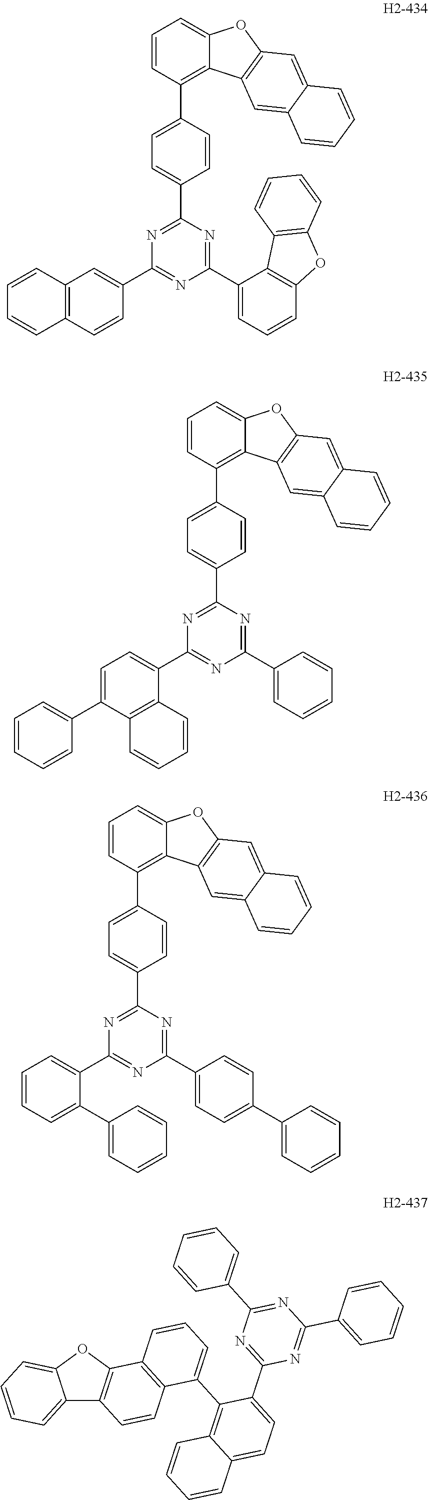

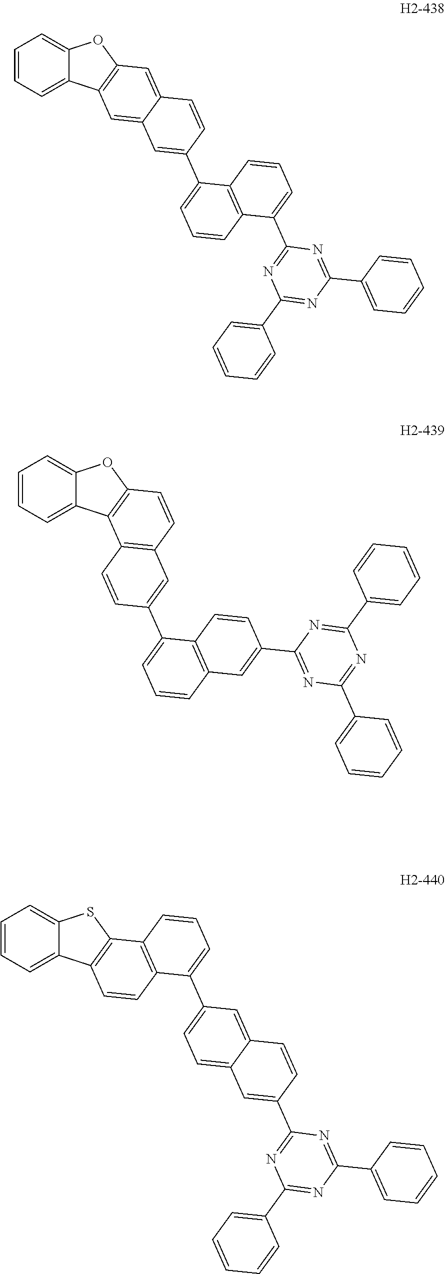 [SMILES: c1ccc(-c2nc(-c3ccccc3)nc(-c3cccc4c(-c5ccc6cc7oc8ccccc8c7cc6c5)cccc34)n2)cc1 ; c1ccc(-c2nc(-c3ccccc3)nc(-c3ccc4c(-c5ccc6c(ccc7oc8ccccc8c76)c5)cccc4c3)n2)cc1 ; c1ccc(-c2nc(-c3ccccc3)nc(-c3ccc4cc(-c5cccc6c5ccc5c7ccccc7sc65)ccc4c3)n2)cc1]

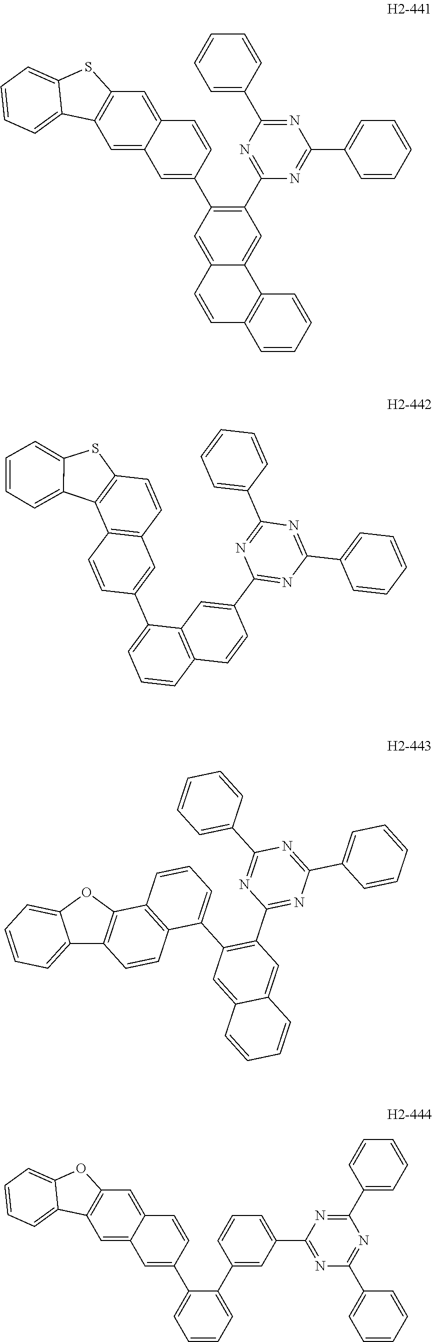 [SMILES: c1ccc(-c2nc(-c3ccccc3)nc(-c3cc4c(ccc5ccccc54)cc3-c3ccc4cc5sc6ccccc6c5cc4c3)n2)cc1 ; c1ccc(-c2nc(-c3ccccc3)nc(-c3ccc4cccc(-c5ccc6c(ccc7sc8ccccc8c76)c5)c4c3)n2)cc1 ; c1ccc(-c2nc(-c3ccccc3)nc(-c3cc4ccccc4cc3-c3cccc4c3ccc3c5ccccc5oc43)n2)cc1 ; c1ccc(-c2nc(-c3ccccc3)nc(-c3cccc(-c4ccccc4-c4ccc5cc6oc7ccccc7c6cc5c4)c3)n2)cc1]

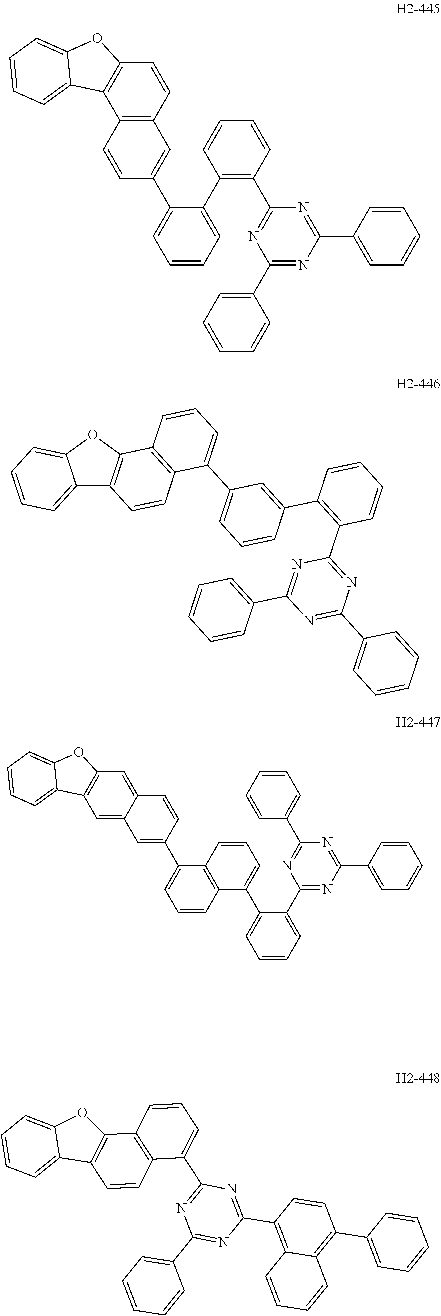 [SMILES: c1ccc(-c2nc(-c3ccccc3)nc(-c3ccccc3-c3ccccc3-c3ccc4c(ccc5oc6ccccc6c54)c3)n2)cc1 ; c1ccc(-c2nc(-c3ccccc3)nc(-c3ccccc3-c3cccc(-c4cccc5c4ccc4c6ccccc6oc54)c3)n2)cc1 ; c1ccc(-c2nc(-c3ccccc3)nc(-c3ccccc3-c3cccc4c(-c5ccc6cc7oc8ccccc8c7cc6c5)cccc34)n2)cc1 ; c1ccc(-c2nc(-c3ccc(-c4ccccc4)c4ccccc34)nc(-c3cccc4c3ccc3c5ccccc5oc43)n2)cc1]

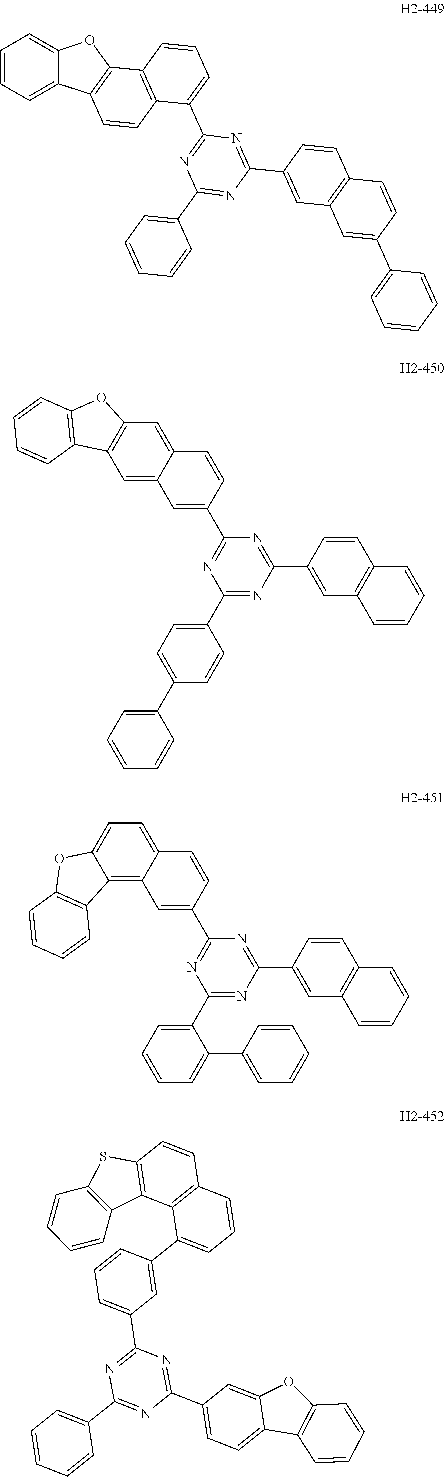 [SMILES: c1ccc(-c2ccc3ccc(-c4nc(-c5ccccc5)nc(-c5cccc6c5ccc5c7ccccc7oc65)n4)cc3c2)cc1 ; c1ccc(-c2ccc(-c3nc(-c4ccc5ccccc5c4)nc(-c4ccc5cc6oc7ccccc7c6cc5c4)n3)cc2)cc1 ; c1ccc(-c2ccccc2-c2nc(-c3ccc4ccccc4c3)nc(-c3ccc4ccc5oc6ccccc6c5c4c3)n2)cc1 ; c1ccc(-c2nc(-c3cccc(-c4cccc5ccc6sc7ccccc7c6c45)c3)nc(-c3ccc4c(c3)oc3ccccc34)n2)cc1]

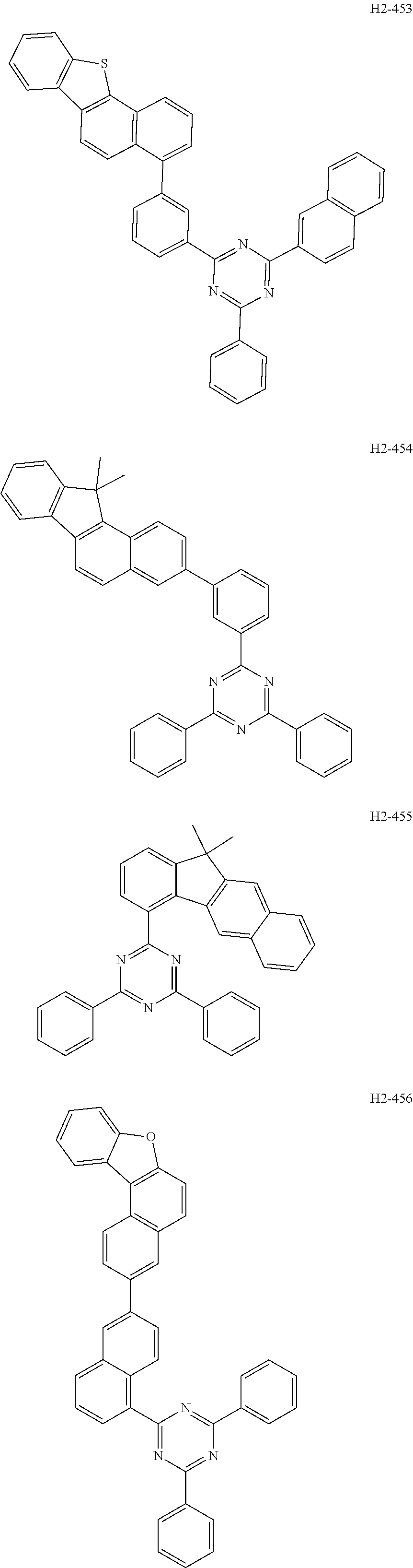 [SMILES: c1ccc(-c2nc(-c3cccc(-c4cccc5c4ccc4c6ccccc6sc54)c3)nc(-c3ccc4ccccc4c3)n2)cc1 ; CC1(C)c2ccccc2-c2ccc3cc(-c4cccc(-c5nc(-c6ccccc6)nc(-c6ccccc6)n5)c4)ccc3c21 ; CC1(C)c2cc3ccccc3cc2-c2c(-c3nc(-c4ccccc4)nc(-c4ccccc4)n3)cccc21 ; c1ccc(-c2nc(-c3ccccc3)nc(-c3cccc4cc(-c5ccc6c(ccc7oc8ccccc8c76)c5)ccc34)n2)cc1]

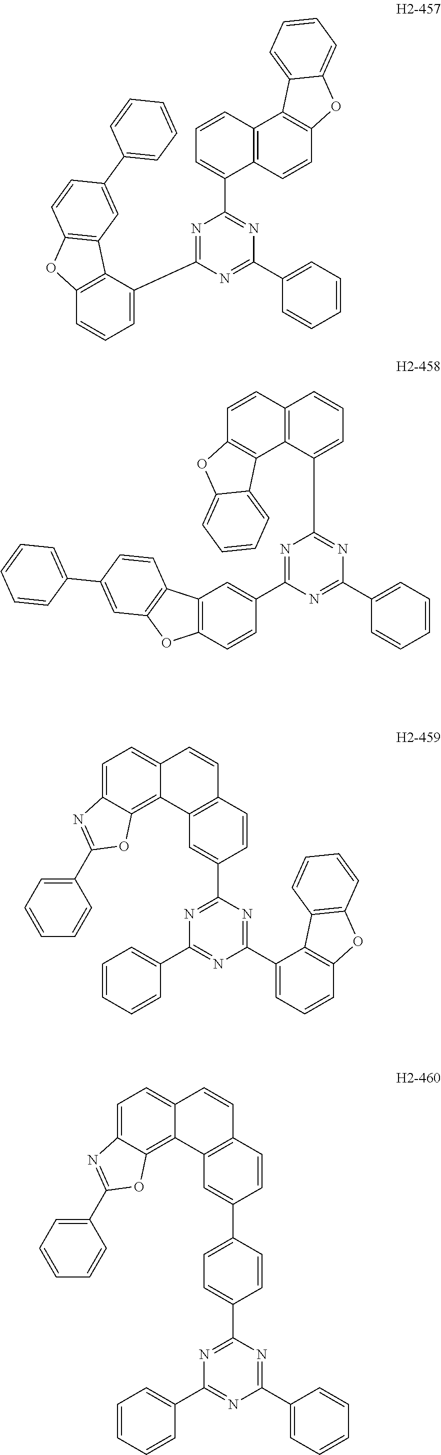 [SMILES: c1ccc(-c2ccc3oc4cccc(-c5nc(-c6ccccc6)nc(-c6cccc7c6ccc6oc8ccccc8c67)n5)c4c3c2)cc1 ; c1ccc(-c2ccc3c(c2)oc2ccc(-c4nc(-c5ccccc5)nc(-c5cccc6ccc7oc8ccccc8c7c56)n4)cc23)cc1 ; c1ccc(-c2nc(-c3ccc4ccc5ccc6nc(-c7ccccc7)oc6c5c4c3)nc(-c3cccc4oc5ccccc5c34)n2)cc1 ; c1ccc(-c2nc(-c3ccccc3)nc(-c3ccc(-c4ccc5ccc6ccc7nc(-c8ccccc8)oc7c6c5c4)cc3)n2)cc1]

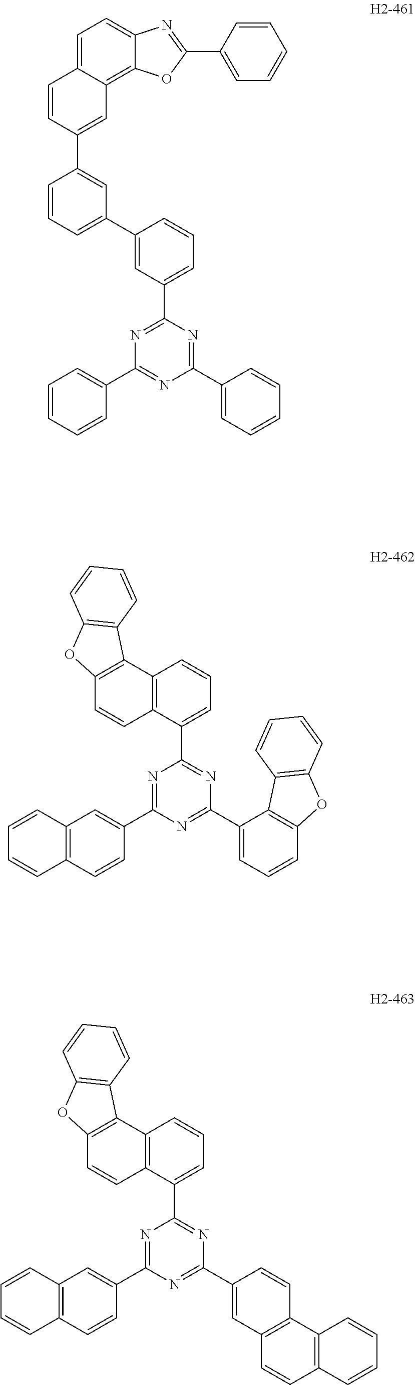 [SMILES: c1ccc(-c2nc(-c3ccccc3)nc(-c3cccc(-c4cccc(-c5ccc6ccc7nc(-c8ccccc8)oc7c6c5)c4)c3)n2)cc1 ; c1ccc2cc(-c3nc(-c4cccc5c4ccc4oc6ccccc6c45)nc(-c4cccc5oc6ccccc6c45)n3)ccc2c1 ; c1ccc2cc(-c3nc(-c4ccc5c(ccc6ccccc65)c4)nc(-c4cccc5c4ccc4oc6ccccc6c45)n3)ccc2c1]

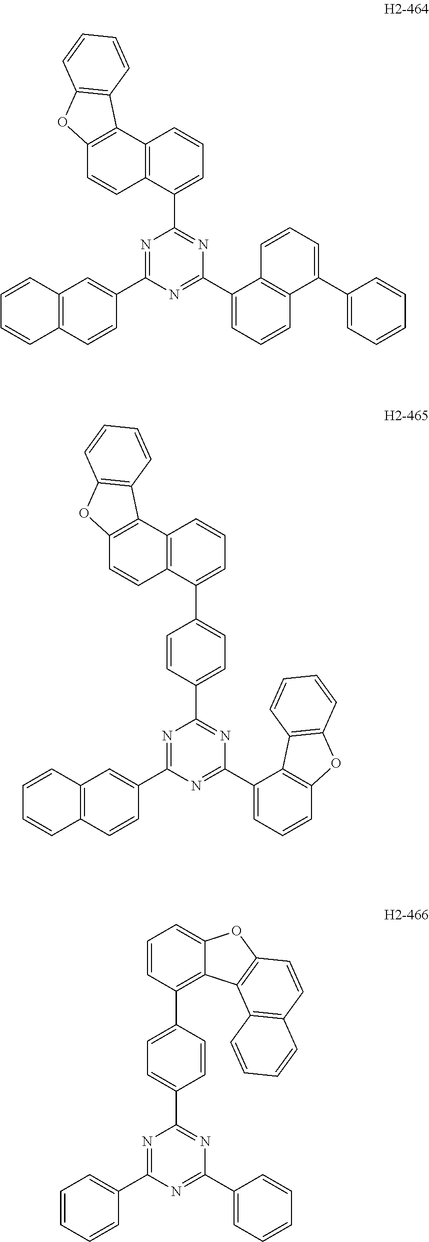 [SMILES: c1ccc(-c2cccc3c(-c4nc(-c5ccc6ccccc6c5)nc(-c5cccc6c5ccc5oc7ccccc7c56)n4)cccc23)cc1 ; c1ccc2cc(-c3nc(-c4ccc(-c5cccc6c5ccc5oc7ccccc7c56)cc4)nc(-c4cccc5oc6ccccc6c45)n3)ccc2c1 ; c1ccc(-c2nc(-c3ccccc3)nc(-c3ccc(-c4cccc5oc6ccc7ccccc7c6c45)cc3)n2)cc1]

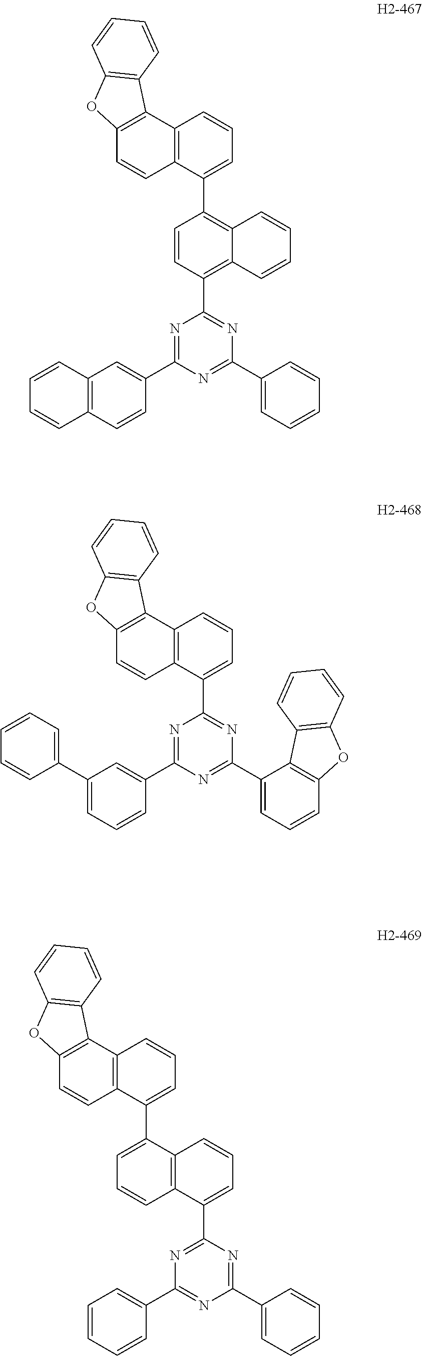 [SMILES: c1ccc(-c2nc(-c3ccc4ccccc4c3)nc(-c3ccc(-c4cccc5c4ccc4oc6ccccc6c45)c4ccccc34)n2)cc1 ; c1ccc(-c2cccc(-c3nc(-c4cccc5c4ccc4oc6ccccc6c45)nc(-c4cccc5oc6ccccc6c45)n3)c2)cc1 ; c1ccc(-c2nc(-c3ccccc3)nc(-c3cccc4c(-c5cccc6c5ccc5oc7ccccc7c56)cccc34)n2)cc1]

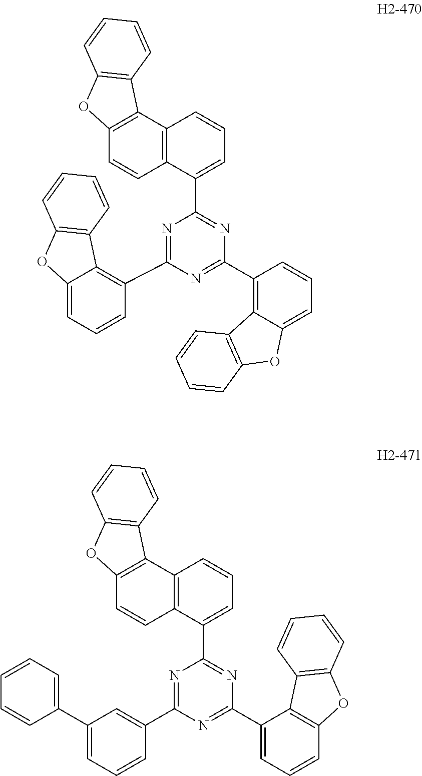 [SMILES: c1ccc2c(c1)oc1cccc(-c3nc(-c4cccc5c4ccc4oc6ccccc6c45)nc(-c4cccc5oc6ccccc6c45)n3)c12 ; c1ccc(-c2cccc(-c3nc(-c4cccc5c4ccc4oc6ccccc6c45)nc(-c4cccc5oc6ccccc6c45)n3)c2)cc1]

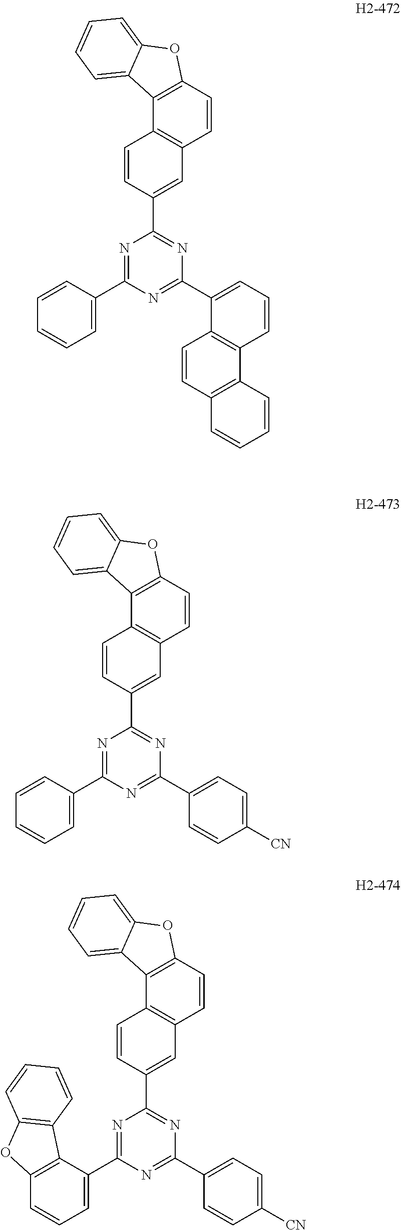 [SMILES: c1ccc(-c2nc(-c3ccc4c(ccc5oc6ccccc6c54)c3)nc(-c3cccc4c3ccc3ccccc34)n2)cc1 ; N#Cc1ccc(-c2nc(-c3ccccc3)nc(-c3ccc4c(ccc5oc6ccccc6c54)c3)n2)cc1 ; N#Cc1ccc(-c2nc(-c3ccc4c(ccc5oc6ccccc6c54)c3)nc(-c3cccc4oc5ccccc5c34)n2)cc1]

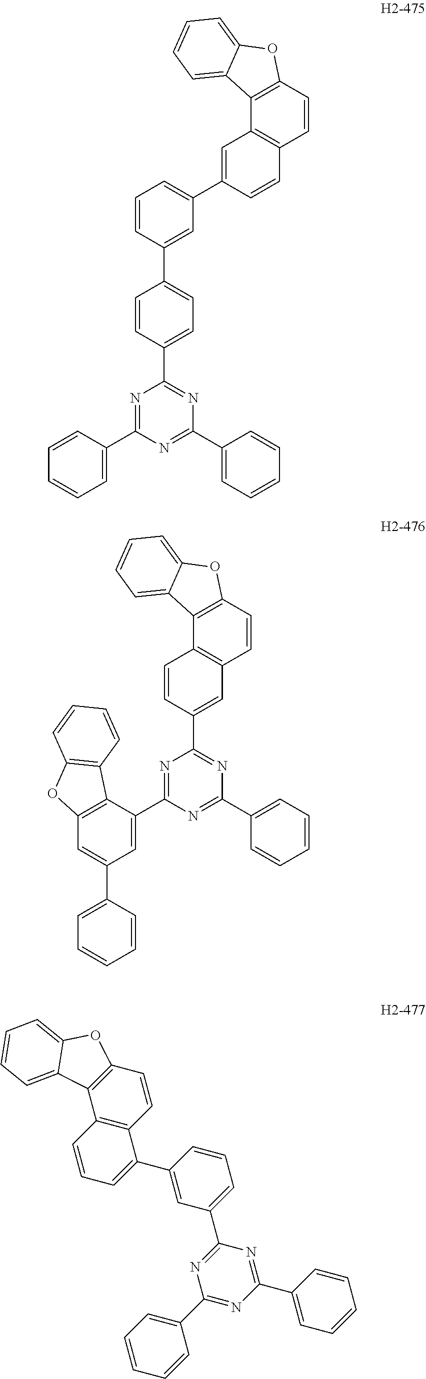 [SMILES: c1ccc(-c2nc(-c3ccccc3)nc(-c3ccc(-c4cccc(-c5ccc6ccc7oc8ccccc8c7c6c5)c4)cc3)n2)cc1 ; c1ccc(-c2cc(-c3nc(-c4ccccc4)nc(-c4ccc5c(ccc6oc7ccccc7c65)c4)n3)c3c(c2)oc2ccccc23)cc1 ; c1ccc(-c2nc(-c3ccccc3)nc(-c3cccc(-c4cccc5c4ccc4oc6ccccc6c45)c3)n2)cc1]

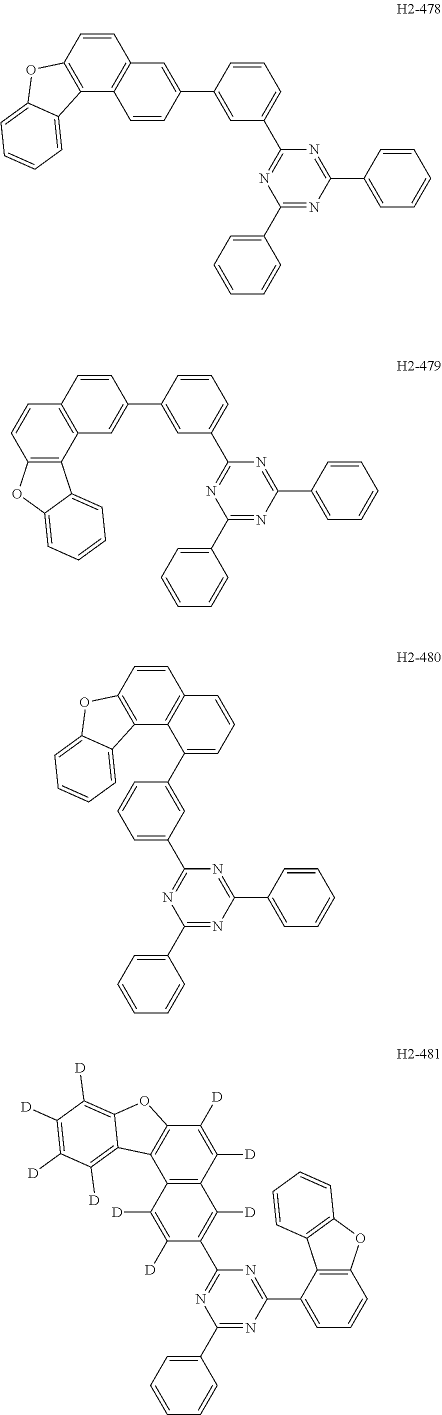 [SMILES: c1ccc(-c2nc(-c3ccccc3)nc(-c3cccc(-c4ccc5c(ccc6oc7ccccc7c65)c4)c3)n2)cc1 ; c1ccc(-c2nc(-c3ccccc3)nc(-c3cccc(-c4ccc5ccc6oc7ccccc7c6c5c4)c3)n2)cc1 ; c1ccc(-c2nc(-c3ccccc3)nc(-c3cccc(-c4cccc5ccc6oc7ccccc7c6c45)c3)n2)cc1 ; [2H]c1c([2H])c([2H])c2c(oc3c([2H])c([2H])c4c([2H])c(-c5nc(-c6ccccc6)nc(-c6cccc7oc8ccccc8c67)n5)c([2H])c([2H])c4c32)c1[2H]]

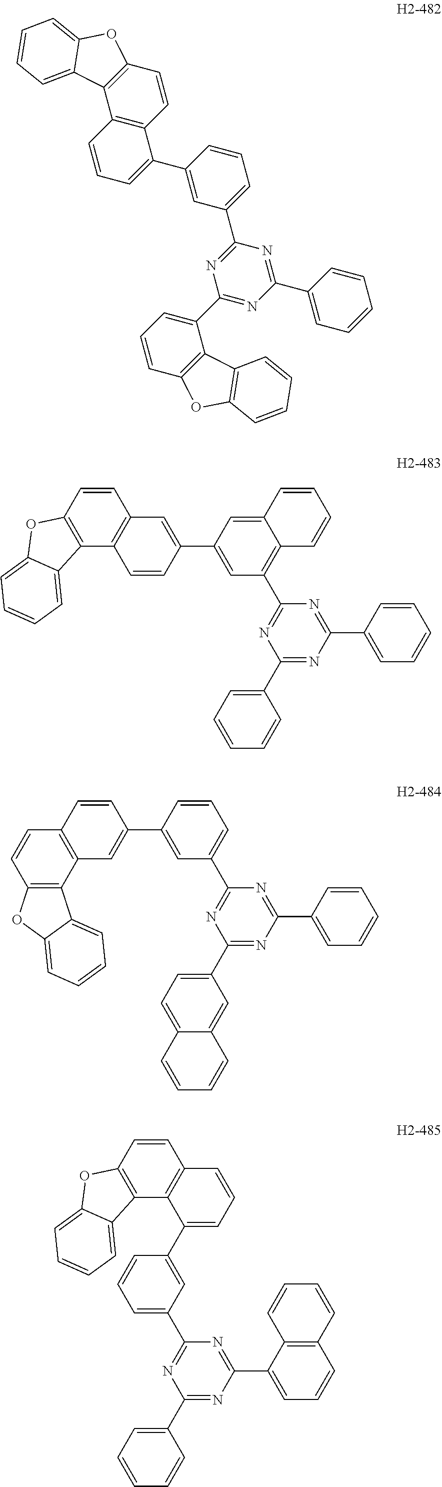 [SMILES: c1ccc(-c2nc(-c3cccc(-c4cccc5c4ccc4oc6ccccc6c45)c3)nc(-c3cccc4oc5ccccc5c34)n2)cc1 ; c1ccc(-c2nc(-c3ccccc3)nc(-c3cc(-c4ccc5c(ccc6oc7ccccc7c65)c4)cc4ccccc34)n2)cc1 ; c1ccc(-c2nc(-c3cccc(-c4ccc5ccc6oc7ccccc7c6c5c4)c3)nc(-c3ccc4ccccc4c3)n2)cc1 ; c1ccc(-c2nc(-c3cccc(-c4cccc5ccc6oc7ccccc7c6c45)c3)nc(-c3cccc4ccccc34)n2)cc1]

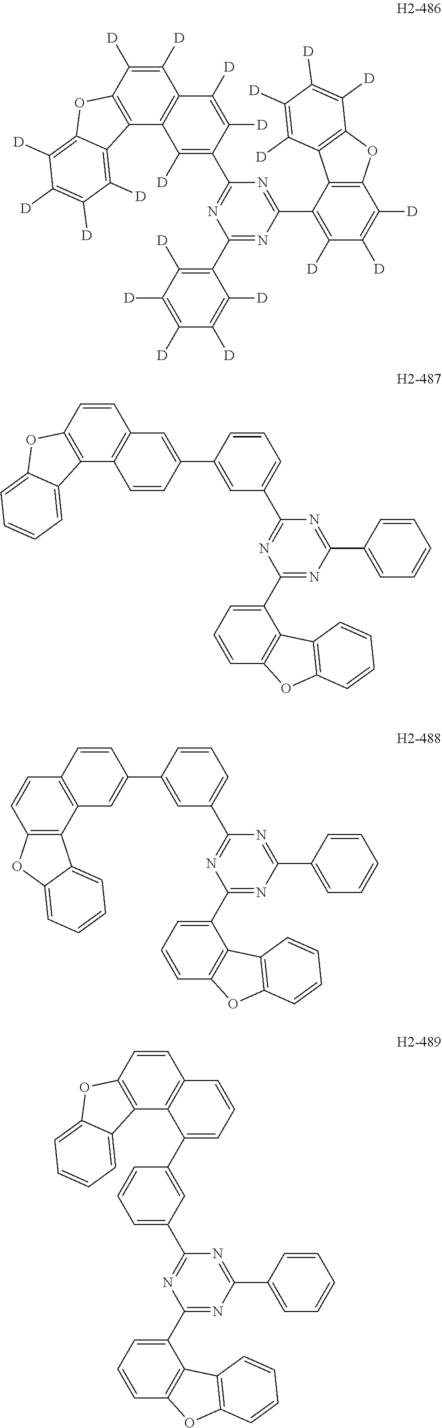 [SMILES: [2H]c1c([2H])c([2H])c(-c2nc(-c3c([2H])c([2H])c4c([2H])c([2H])c5oc6c([2H])c([2H])c([2H])c([2H])c6c5c4c3[2H])nc(-c3c([2H])c([2H])c([2H])c4oc5c([2H])c([2H])c([2H])c([2H])c5c34)n2)c([2H])c1[2H] ; c1ccc(-c2nc(-c3cccc(-c4ccc5c(ccc6oc7ccccc7c65)c4)c3)nc(-c3cccc4oc5ccccc5c34)n2)cc1 ; c1ccc(-c2nc(-c3cccc(-c4ccc5ccc6oc7ccccc7c6c5c4)c3)nc(-c3cccc4oc5ccccc5c34)n2)cc1 ; c1ccc(-c2nc(-c3cccc(-c4cccc5ccc6oc7ccccc7c6c45)c3)nc(-c3cccc4oc5ccccc5c34)n2)cc1]

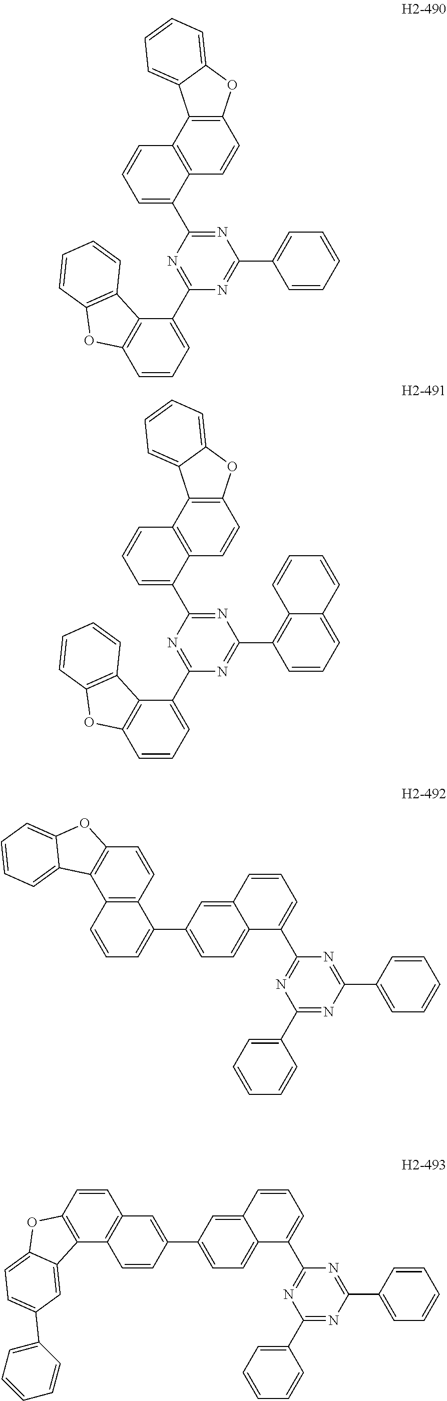

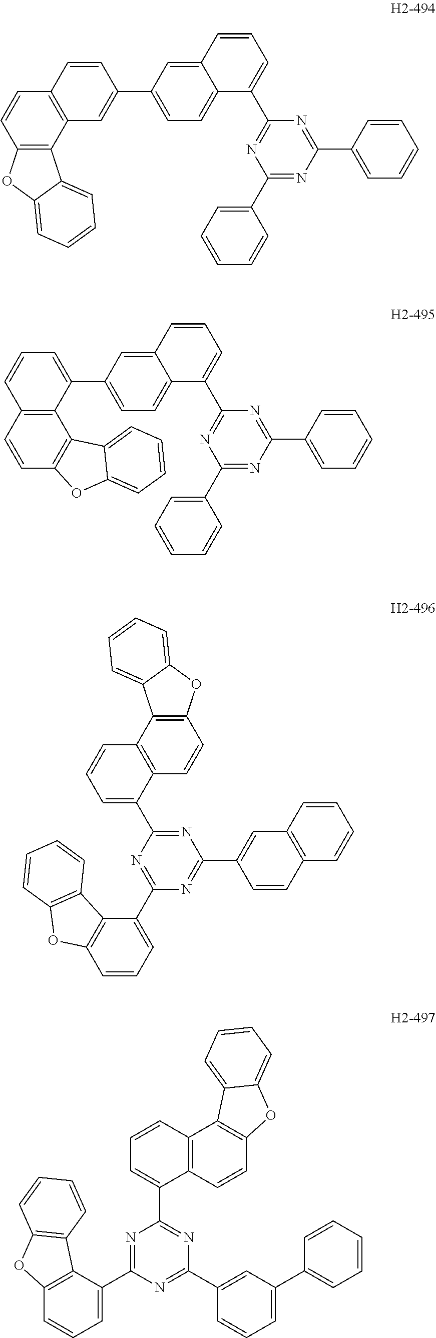 [SMILES: c1ccc(-c2nc(-c3ccccc3)nc(-c3cccc4cc(-c5ccc6ccc7oc8ccccc8c7c6c5)ccc34)n2)cc1 ; c1ccc(-c2nc(-c3ccccc3)nc(-c3cccc4cc(-c5cccc6ccc7oc8ccccc8c7c56)ccc34)n2)cc1 ; c1ccc2cc(-c3nc(-c4cccc5c4ccc4oc6ccccc6c45)nc(-c4cccc5oc6ccccc6c45)n3)ccc2c1 ; c1ccc(-c2cccc(-c3nc(-c4cccc5c4ccc4oc6ccccc6c45)nc(-c4cccc5oc6ccccc6c45)n3)c2)cc1]

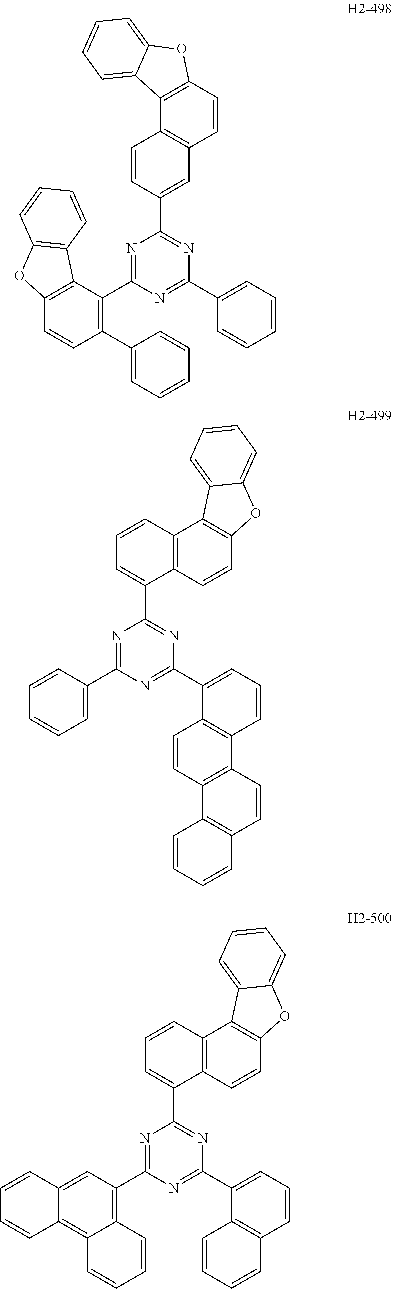 [SMILES: c1ccc(-c2nc(-c3ccc4c(ccc5oc6ccccc6c54)c3)nc(-c3c(-c4ccccc4)ccc4oc5ccccc5c34)n2)cc1 ; c1ccc(-c2nc(-c3cccc4c3ccc3c5ccccc5ccc43)nc(-c3cccc4c3ccc3oc5ccccc5c34)n2)cc1 ; c1ccc2c(-c3nc(-c4cc5ccccc5c5ccccc45)nc(-c4cccc5c4ccc4oc6ccccc6c45)n3)cccc2c1]

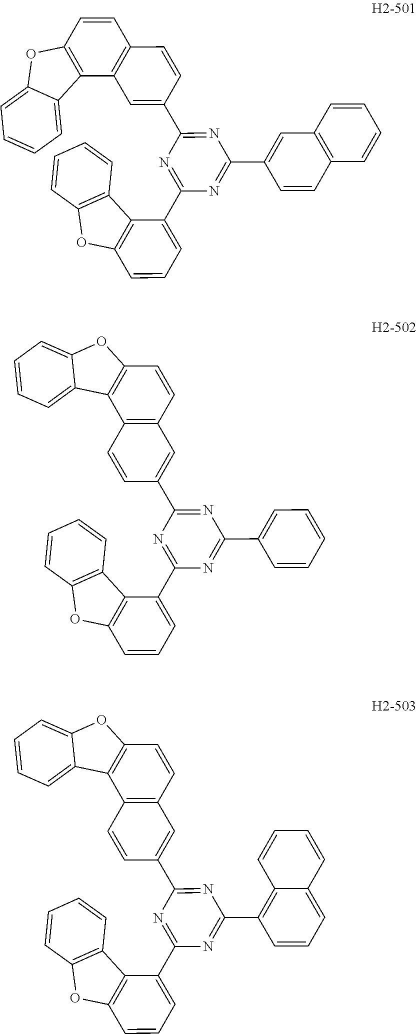 [SMILES: c1ccc2cc(-c3nc(-c4ccc5ccc6oc7ccccc7c6c5c4)nc(-c4cccc5oc6ccccc6c45)n3)ccc2c1 ; c1ccc(-c2nc(-c3ccc4c(ccc5oc6ccccc6c54)c3)nc(-c3cccc4oc5ccccc5c34)n2)cc1 ; c1ccc2c(-c3nc(-c4ccc5c(ccc6oc7ccccc7c65)c4)nc(-c4cccc5oc6ccccc6c45)n3)cccc2c1]

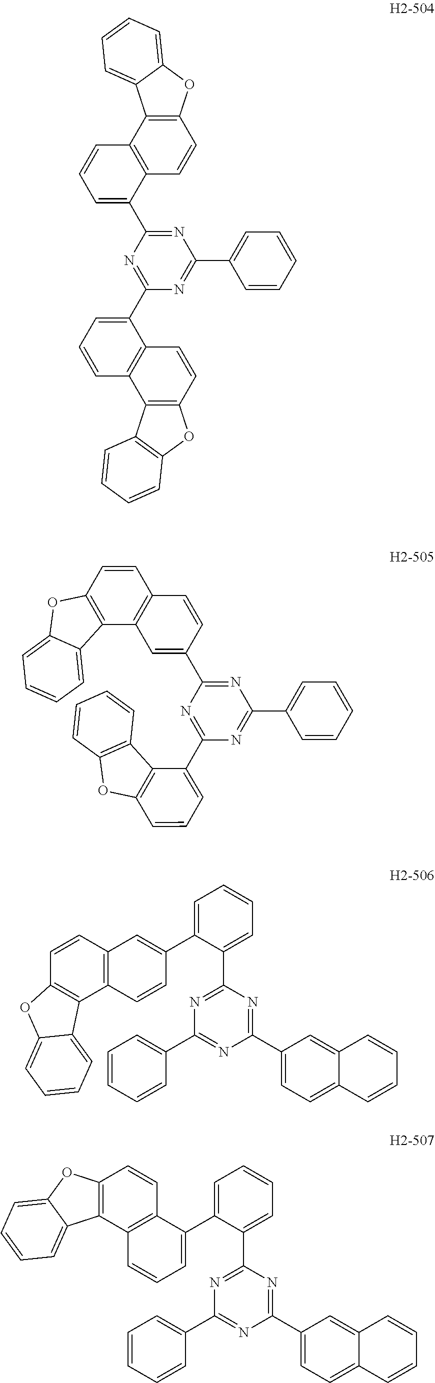 [SMILES: c1ccc(-c2nc(-c3cccc4c3ccc3oc5ccccc5c34)nc(-c3cccc4c3ccc3oc5ccccc5c34)n2)cc1 ; c1ccc(-c2nc(-c3ccc4ccc5oc6ccccc6c5c4c3)nc(-c3cccc4oc5ccccc5c34)n2)cc1 ; c1ccc(-c2nc(-c3ccc4ccccc4c3)nc(-c3ccccc3-c3ccc4c(ccc5oc6ccccc6c54)c3)n2)cc1 ; c1ccc(-c2nc(-c3ccc4ccccc4c3)nc(-c3ccccc3-c3cccc4c3ccc3oc5ccccc5c34)n2)cc1]

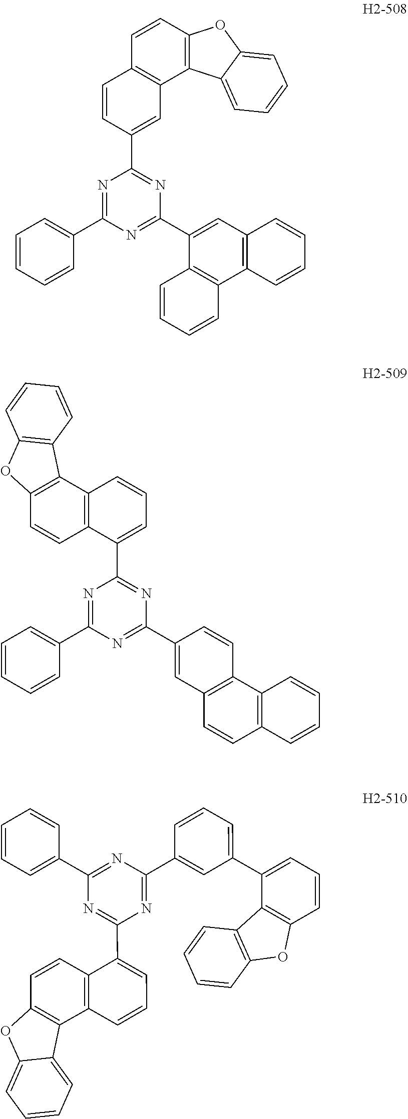 [SMILES: c1ccc(-c2nc(-c3ccc4ccc5oc6ccccc6c5c4c3)nc(-c3cc4ccccc4c4ccccc34)n2)cc1 ; c1ccc(-c2nc(-c3ccc4c(ccc5ccccc54)c3)nc(-c3cccc4c3ccc3oc5ccccc5c34)n2)cc1 ; c1ccc(-c2nc(-c3cccc(-c4cccc5oc6ccccc6c45)c3)nc(-c3cccc4c3ccc3oc5ccccc5c34)n2)cc1]

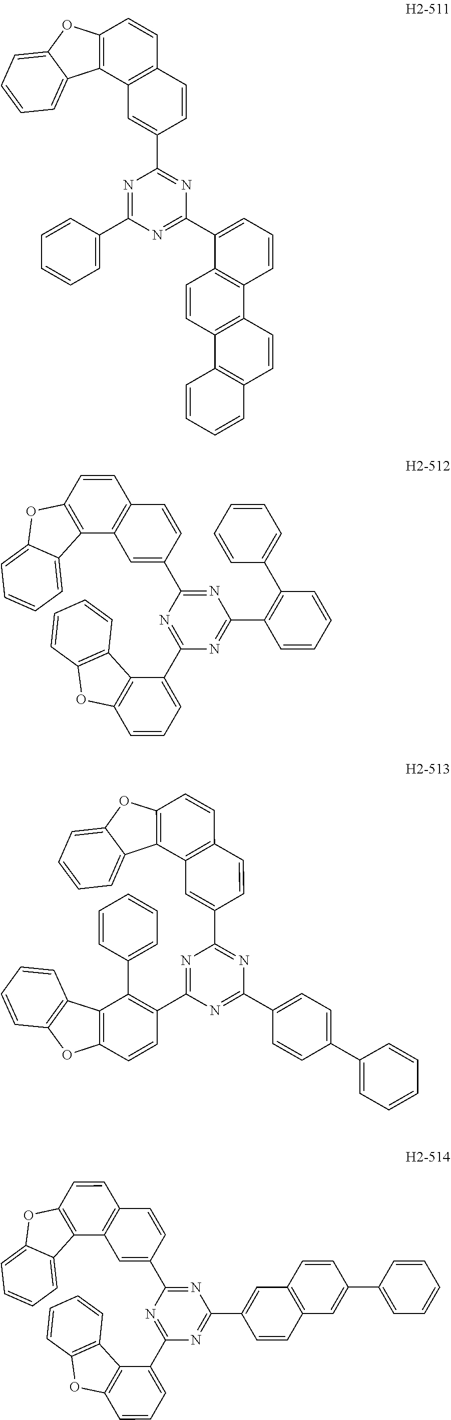 [SMILES: c1ccc(-c2nc(-c3ccc4ccc5oc6ccccc6c5c4c3)nc(-c3cccc4c3ccc3c5ccccc5ccc43)n2)cc1 ; c1ccc(-c2ccccc2-c2nc(-c3ccc4ccc5oc6ccccc6c5c4c3)nc(-c3cccc4oc5ccccc5c34)n2)cc1 ; c1ccc(-c2ccc(-c3nc(-c4ccc5ccc6oc7ccccc7c6c5c4)nc(-c4ccc5oc6ccccc6c5c4-c4ccccc4)n3)cc2)cc1 ; c1ccc(-c2ccc3cc(-c4nc(-c5ccc6ccc7oc8ccccc8c7c6c5)nc(-c5cccc6oc7ccccc7c56)n4)ccc3c2)cc1]

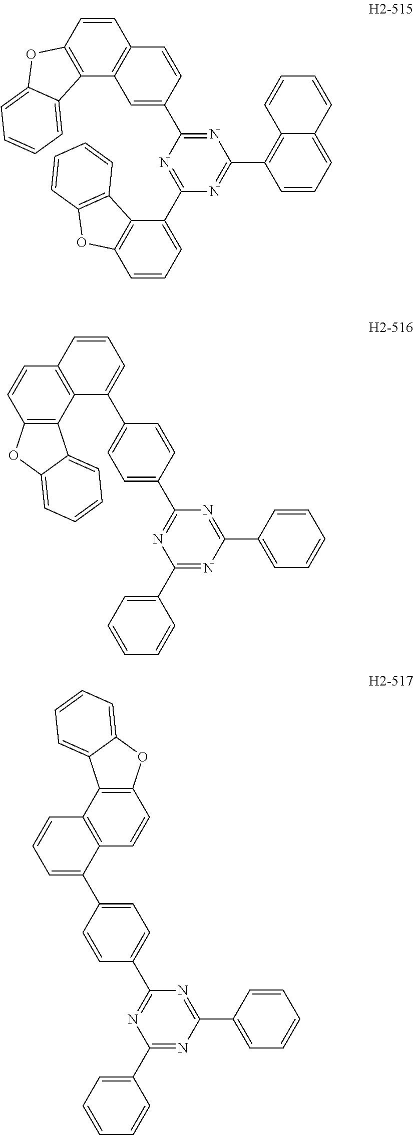 [SMILES: c1ccc2c(-c3nc(-c4ccc5ccc6oc7ccccc7c6c5c4)nc(-c4cccc5oc6ccccc6c45)n3)cccc2c1 ; c1ccc(-c2nc(-c3ccccc3)nc(-c3ccc(-c4cccc5ccc6oc7ccccc7c6c45)cc3)n2)cc1 ; c1ccc(-c2nc(-c3ccccc3)nc(-c3ccc(-c4cccc5c4ccc4oc6ccccc6c45)cc3)n2)cc1]

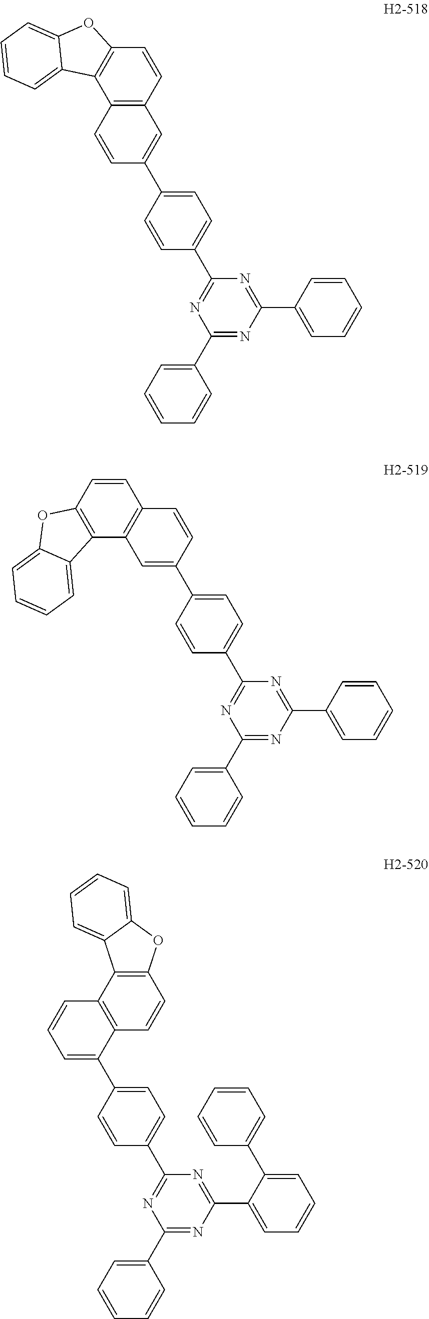 [SMILES: c1ccc(-c2nc(-c3ccccc3)nc(-c3ccc(-c4ccc5c(ccc6oc7ccccc7c65)c4)cc3)n2)cc1 ; c1ccc(-c2nc(-c3ccccc3)nc(-c3ccc(-c4ccc5ccc6oc7ccccc7c6c5c4)cc3)n2)cc1 ; c1ccc(-c2nc(-c3ccc(-c4cccc5c4ccc4oc6ccccc6c45)cc3)nc(-c3ccccc3-c3ccccc3)n2)cc1]

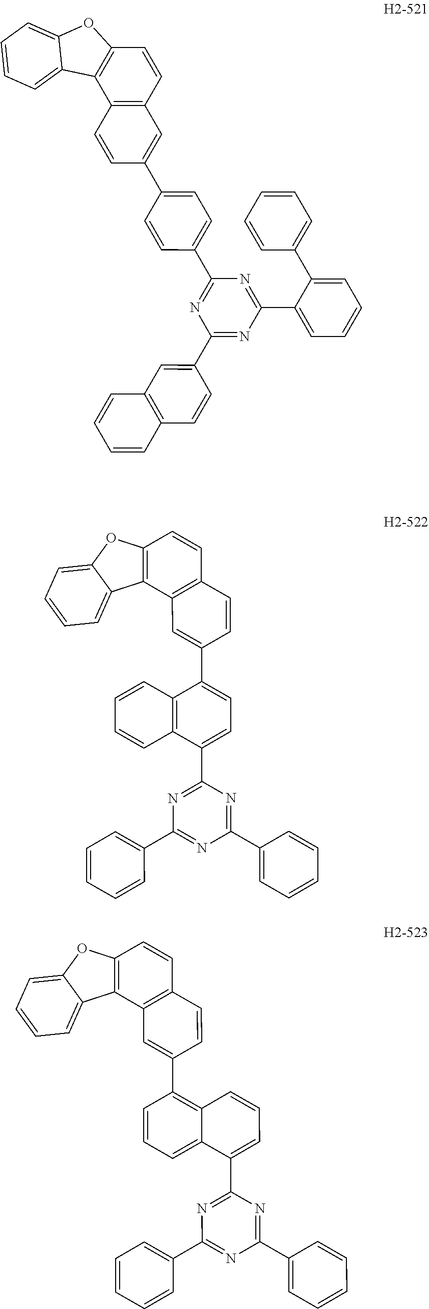 [SMILES: c1ccc(-c2ccccc2-c2nc(-c3ccc(-c4ccc5c(ccc6oc7ccccc7c65)c4)cc3)nc(-c3ccc4ccccc4c3)n2)cc1 ; c1ccc(-c2nc(-c3ccccc3)nc(-c3ccc(-c4ccc5ccc6oc7ccccc7c6c5c4)c4ccccc34)n2)cc1 ; c1ccc(-c2nc(-c3ccccc3)nc(-c3cccc4c(-c5ccc6ccc7oc8ccccc8c7c6c5)cccc34)n2)cc1]

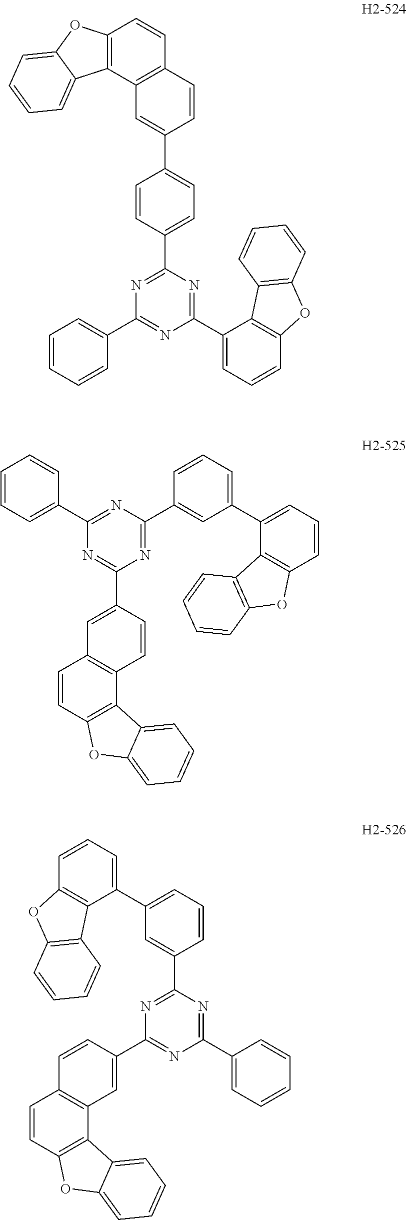 [SMILES: c1ccc(-c2nc(-c3ccc(-c4ccc5ccc6oc7ccccc7c6c5c4)cc3)nc(-c3cccc4oc5ccccc5c34)n2)cc1 ; c1ccc(-c2nc(-c3cccc(-c4cccc5oc6ccccc6c45)c3)nc(-c3ccc4c(ccc5oc6ccccc6c54)c3)n2)cc1 ; c1ccc(-c2nc(-c3cccc(-c4cccc5oc6ccccc6c45)c3)nc(-c3ccc4ccc5oc6ccccc6c5c4c3)n2)cc1]

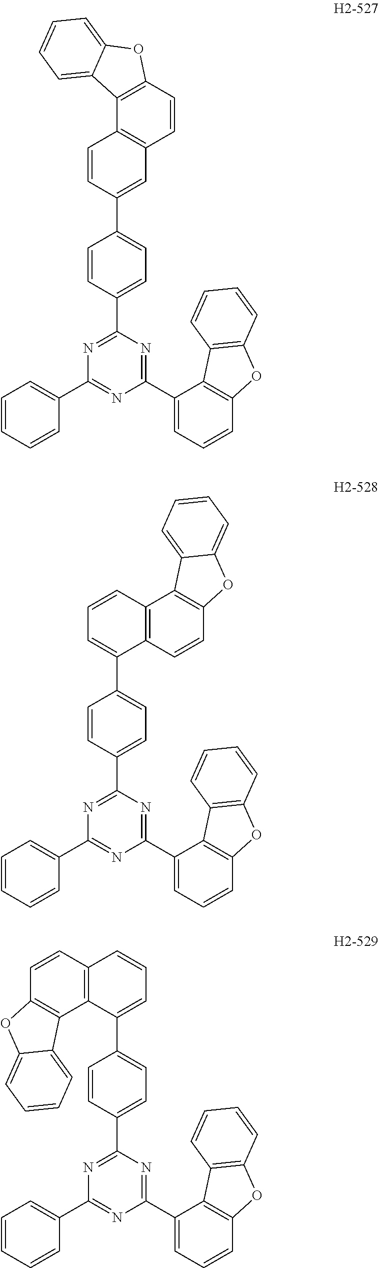 [SMILES: c1ccc(-c2nc(-c3ccc(-c4ccc5c(ccc6oc7ccccc7c65)c4)cc3)nc(-c3cccc4oc5ccccc5c34)n2)cc1 ; c1ccc(-c2nc(-c3ccc(-c4cccc5c4ccc4oc6ccccc6c45)cc3)nc(-c3cccc4oc5ccccc5c34)n2)cc1 ; c1ccc(-c2nc(-c3ccc(-c4cccc5ccc6oc7ccccc7c6c45)cc3)nc(-c3cccc4oc5ccccc5c34)n2)cc1]

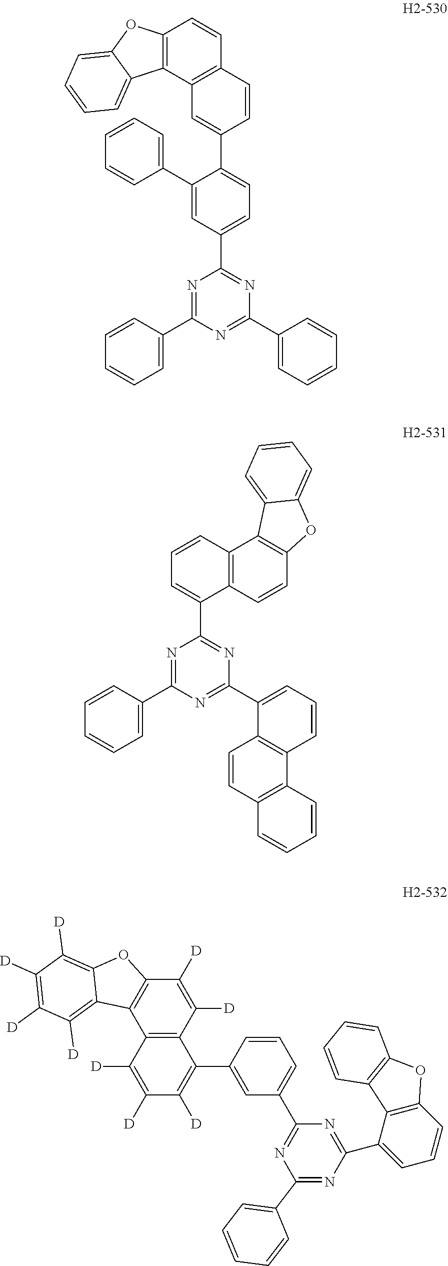 [SMILES: c1ccc(-c2nc(-c3ccccc3)nc(-c3ccc(-c4ccc5ccc6oc7ccccc7c6c5c4)c(-c4ccccc4)c3)n2)cc1 ; c1ccc(-c2nc(-c3cccc4c3ccc3ccccc34)nc(-c3cccc4c3ccc3oc5ccccc5c34)n2)cc1 ; [2H]c1c([2H])c([2H])c2c(oc3c([2H])c([2H])c4c(-c5cccc(-c6nc(-c7ccccc7)nc(-c7cccc8oc9ccccc9c78)n6)c5)c([2H])c([2H])c([2H])c4c32)c1[2H]]

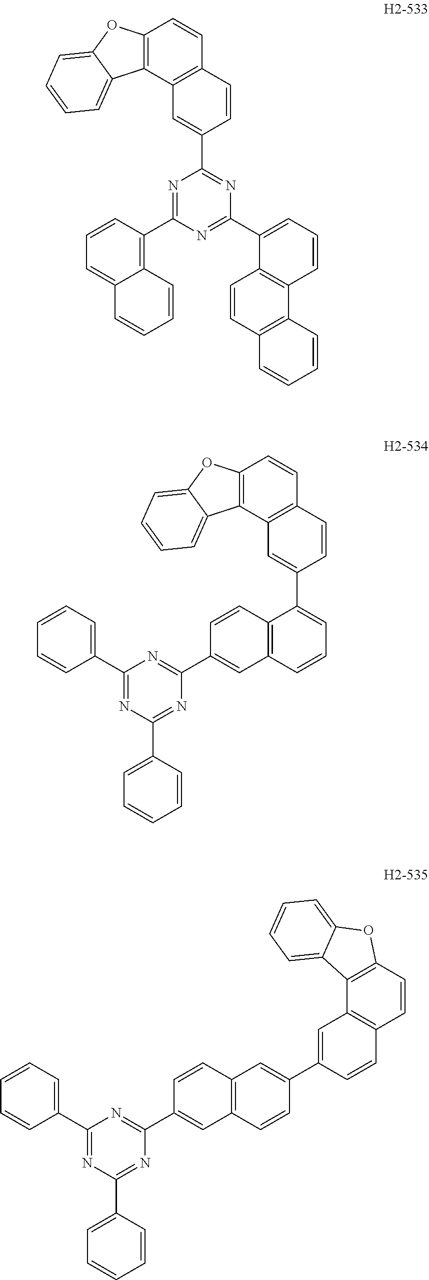 [SMILES: c1ccc2c(-c3nc(-c4ccc5ccc6oc7ccccc7c6c5c4)nc(-c4cccc5c4ccc4ccccc45)n3)cccc2c1 ; c1ccc(-c2nc(-c3ccccc3)nc(-c3ccc4c(-c5ccc6ccc7oc8ccccc8c7c6c5)cccc4c3)n2)cc1 ; c1ccc(-c2nc(-c3ccccc3)nc(-c3ccc4cc(-c5ccc6ccc7oc8ccccc8c7c6c5)ccc4c3)n2)cc1]

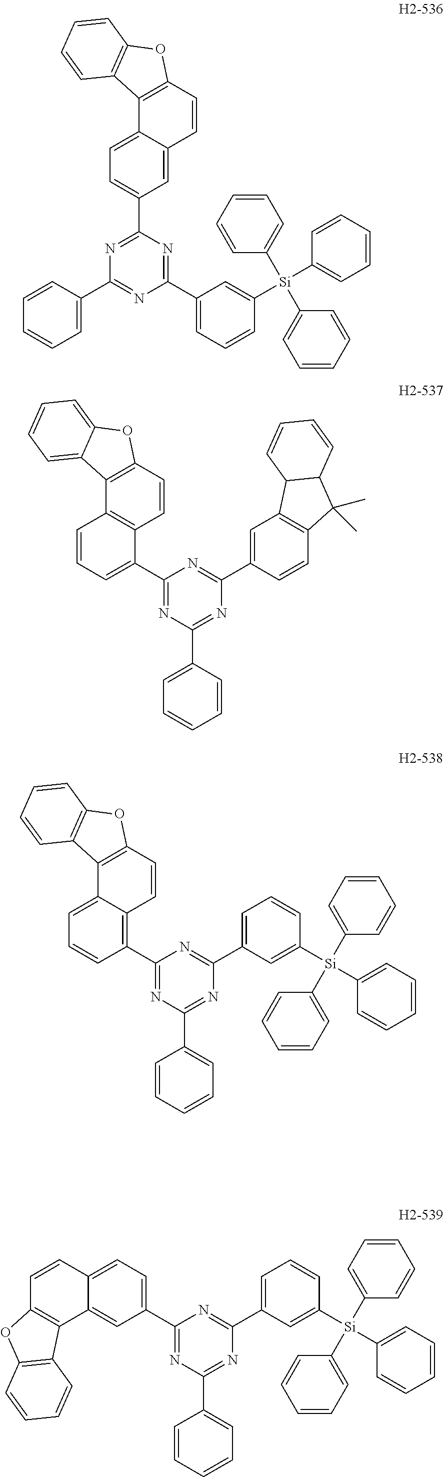 [SMILES: c1ccc(-c2nc(-c3cccc([Si](c4ccccc4)(c4ccccc4)c4ccccc4)c3)nc(-c3ccc4c(ccc5oc6ccccc6c54)c3)n2)cc1 ; CC1(C)c2ccc(-c3nc(-c4ccccc4)nc(-c4cccc5c4ccc4oc6ccccc6c45)n3)cc2C2C=CC=CC21 ; c1ccc(-c2nc(-c3cccc([Si](c4ccccc4)(c4ccccc4)c4ccccc4)c3)nc(-c3cccc4c3ccc3oc5ccccc5c34)n2)cc1 ; c1ccc(-c2nc(-c3cccc([Si](c4ccccc4)(c4ccccc4)c4ccccc4)c3)nc(-c3ccc4ccc5oc6ccccc6c5c4c3)n2)cc1]

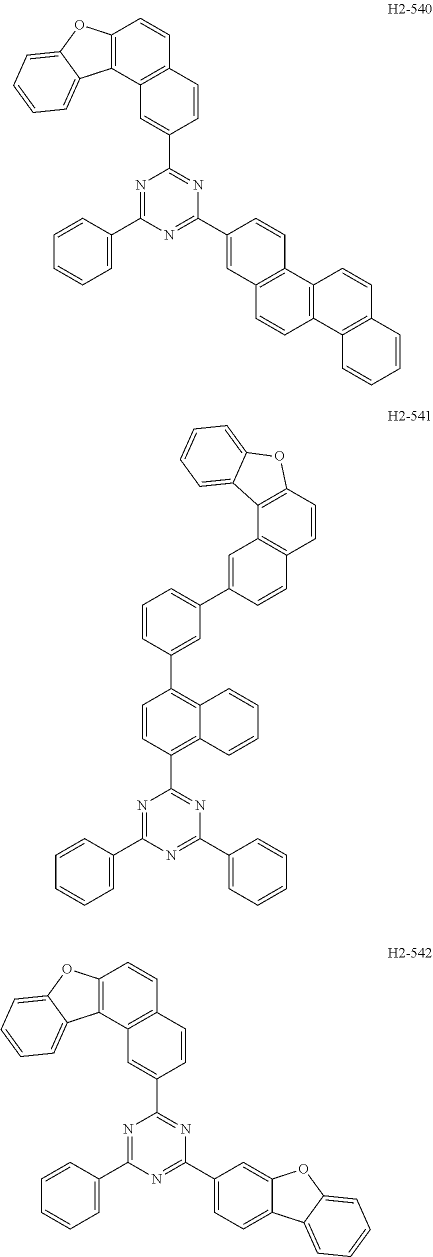 [SMILES: c1ccc(-c2nc(-c3ccc4c(ccc5c6ccccc6ccc45)c3)nc(-c3ccc4ccc5oc6ccccc6c5c4c3)n2)cc1 ; c1ccc(-c2nc(-c3ccccc3)nc(-c3ccc(-c4cccc(-c5ccc6ccc7oc8ccccc8c7c6c5)c4)c4ccccc34)n2)cc1 ; c1ccc(-c2nc(-c3ccc4c(c3)oc3ccccc34)nc(-c3ccc4ccc5oc6ccccc6c5c4c3)n2)cc1]

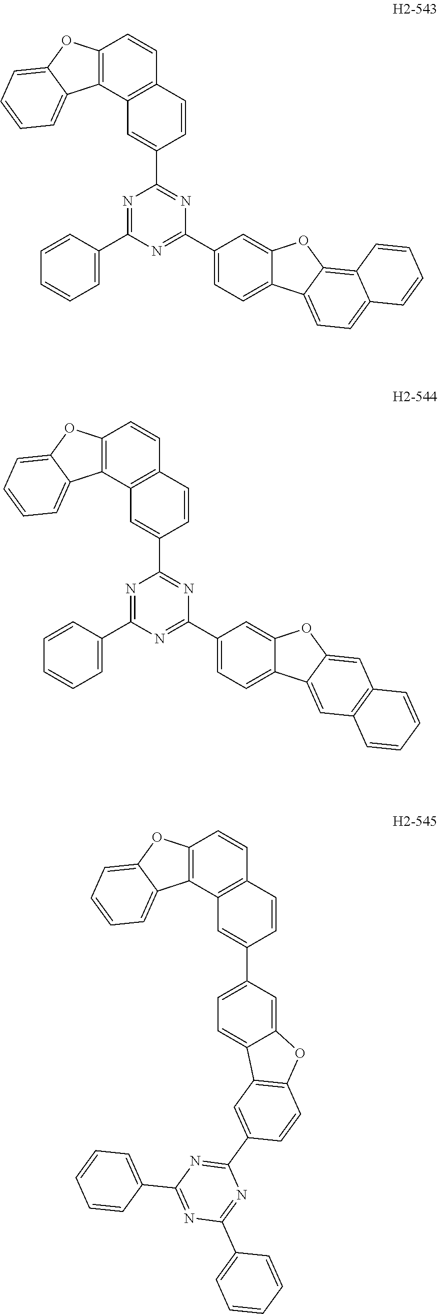 [SMILES: c1ccc(-c2nc(-c3ccc4c(c3)oc3c5ccccc5ccc43)nc(-c3ccc4ccc5oc6ccccc6c5c4c3)n2)cc1 ; c1ccc(-c2nc(-c3ccc4c(c3)oc3cc5ccccc5cc34)nc(-c3ccc4ccc5oc6ccccc6c5c4c3)n2)cc1 ; c1ccc(-c2nc(-c3ccccc3)nc(-c3ccc4oc5cc(-c6ccc7ccc8oc9ccccc9c8c7c6)ccc5c4c3)n2)cc1]

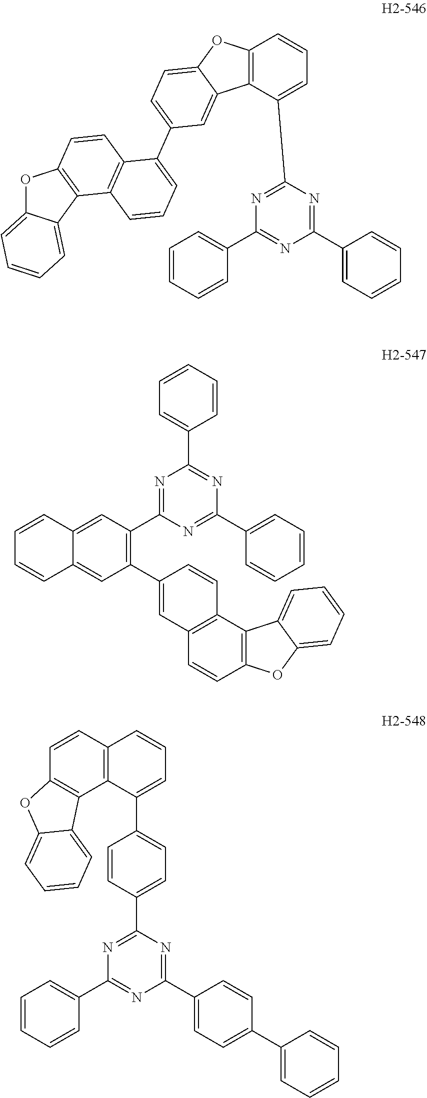 [SMILES: c1ccc(-c2nc(-c3ccccc3)nc(-c3cccc4oc5ccc(-c6cccc7c6ccc6oc8ccccc8c67)cc5c34)n2)cc1 ; c1ccc(-c2nc(-c3ccccc3)nc(-c3cc4ccccc4cc3-c3ccc4c(ccc5oc6ccccc6c54)c3)n2)cc1 ; c1ccc(-c2ccc(-c3nc(-c4ccccc4)nc(-c4ccc(-c5cccc6ccc7oc8ccccc8c7c56)cc4)n3)cc2)cc1]

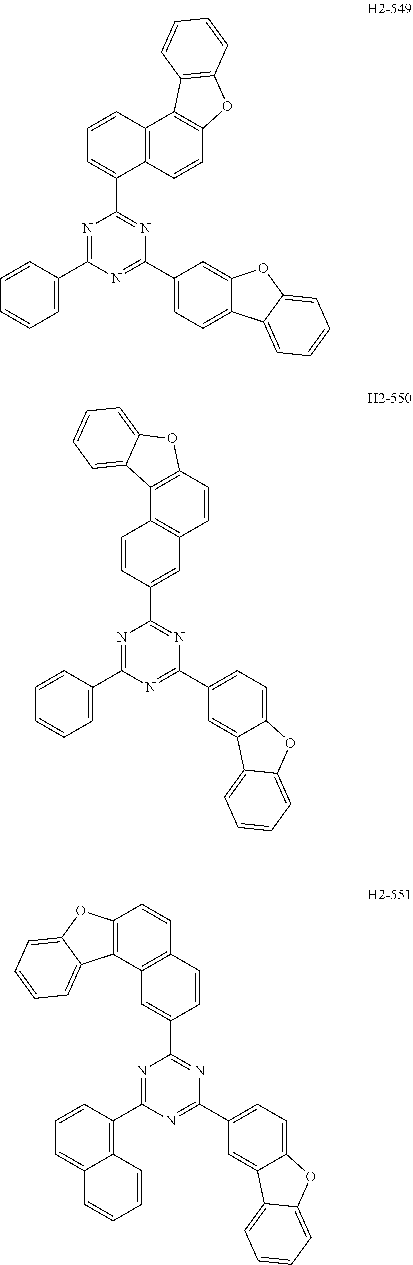 [SMILES: c1ccc(-c2nc(-c3ccc4c(c3)oc3ccccc34)nc(-c3cccc4c3ccc3oc5ccccc5c34)n2)cc1 ; c1ccc(-c2nc(-c3ccc4c(ccc5oc6ccccc6c54)c3)nc(-c3ccc4oc5ccccc5c4c3)n2)cc1 ; c1ccc2c(-c3nc(-c4ccc5oc6ccccc6c5c4)nc(-c4ccc5ccc6oc7ccccc7c6c5c4)n3)cccc2c1]

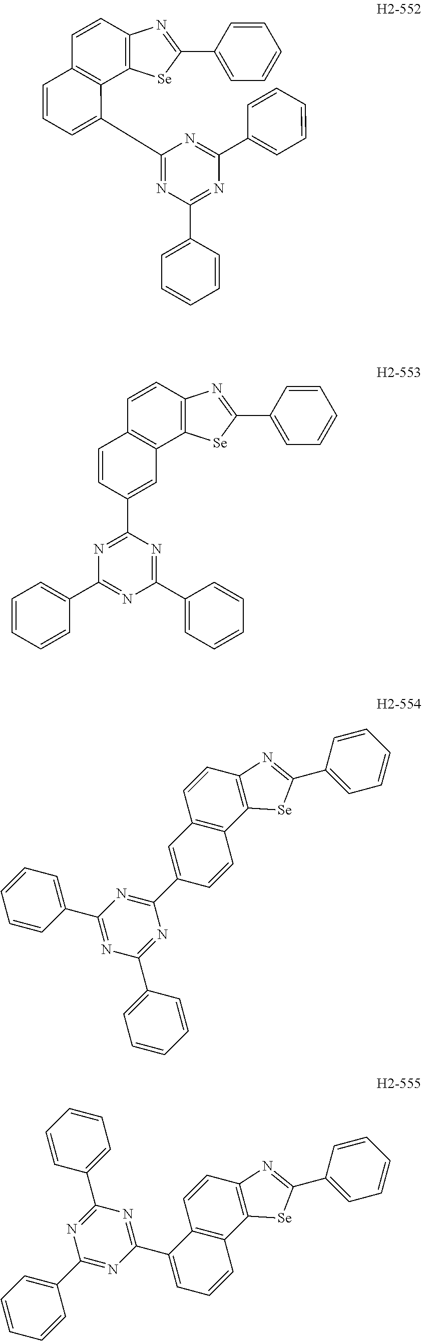 [SMILES: c1ccc(-c2nc(-c3ccccc3)nc(-c3cccc4ccc5nc(-c6ccccc6)[se]c5c34)n2)cc1 ; c1ccc(-c2nc(-c3ccccc3)nc(-c3ccc4ccc5nc(-c6ccccc6)[se]c5c4c3)n2)cc1 ; c1ccc(-c2nc(-c3ccccc3)nc(-c3ccc4c(ccc5nc(-c6ccccc6)[se]c54)c3)n2)cc1 ; c1ccc(-c2nc(-c3ccccc3)nc(-c3cccc4c3ccc3nc(-c5ccccc5)[se]c34)n2)cc1]

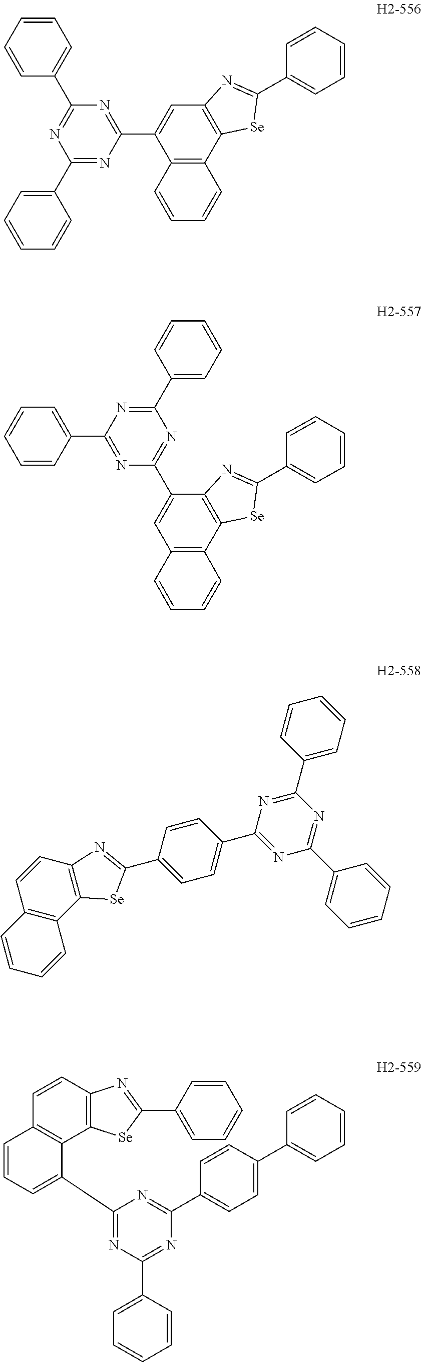 [SMILES: c1ccc(-c2nc(-c3ccccc3)nc(-c3cc4nc(-c5ccccc5)[se]c4c4ccccc34)n2)cc1 ; c1ccc(-c2nc(-c3ccccc3)nc(-c3cc4ccccc4c4[se]c(-c5ccccc5)nc34)n2)cc1 ; c1ccc(-c2nc(-c3ccccc3)nc(-c3ccc(-c4nc5ccc6ccccc6c5[se]4)cc3)n2)cc1 ; c1ccc(-c2ccc(-c3nc(-c4ccccc4)nc(-c4cccc5ccc6nc(-c7ccccc7)[se]c6c45)n3)cc2)cc1]

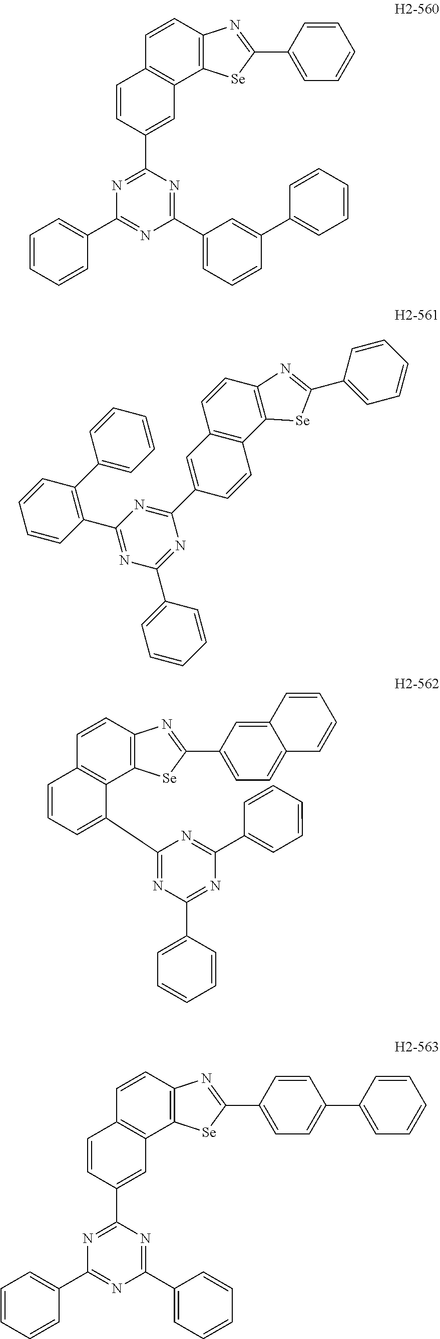 [SMILES: c1ccc(-c2cccc(-c3nc(-c4ccccc4)nc(-c4ccc5ccc6nc(-c7ccccc7)[se]c6c5c4)n3)c2)cc1 ; c1ccc(-c2nc(-c3ccc4c(ccc5nc(-c6ccccc6)[se]c54)c3)nc(-c3ccccc3-c3ccccc3)n2)cc1 ; c1ccc(-c2nc(-c3ccccc3)nc(-c3cccc4ccc5nc(-c6ccc7ccccc7c6)[se]c5c34)n2)cc1 ; c1ccc(-c2ccc(-c3nc4ccc5ccc(-c6nc(-c7ccccc7)nc(-c7ccccc7)n6)cc5c4[se]3)cc2)cc1]

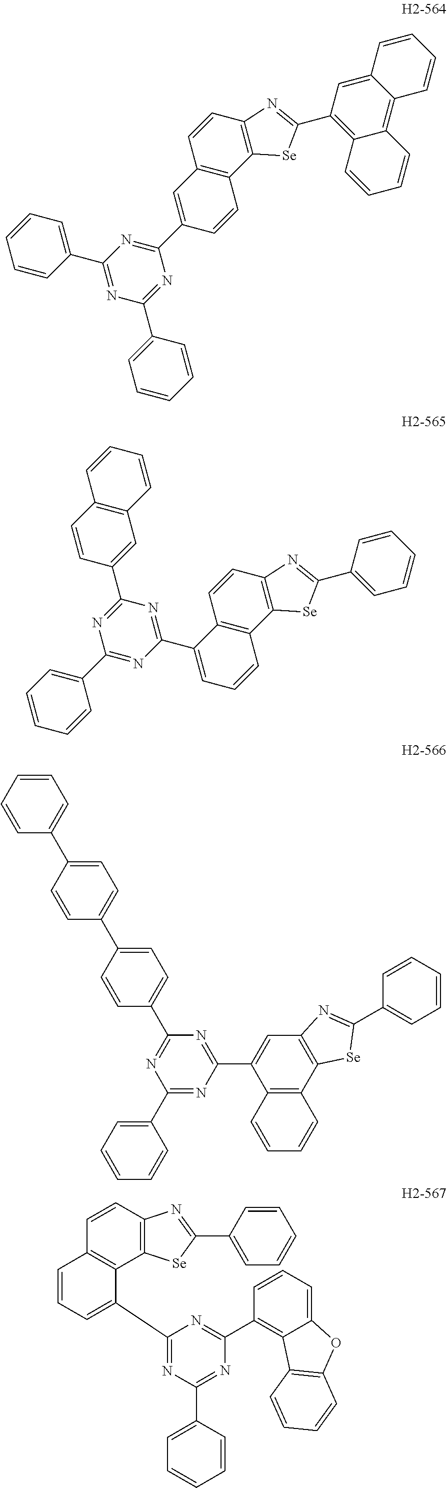

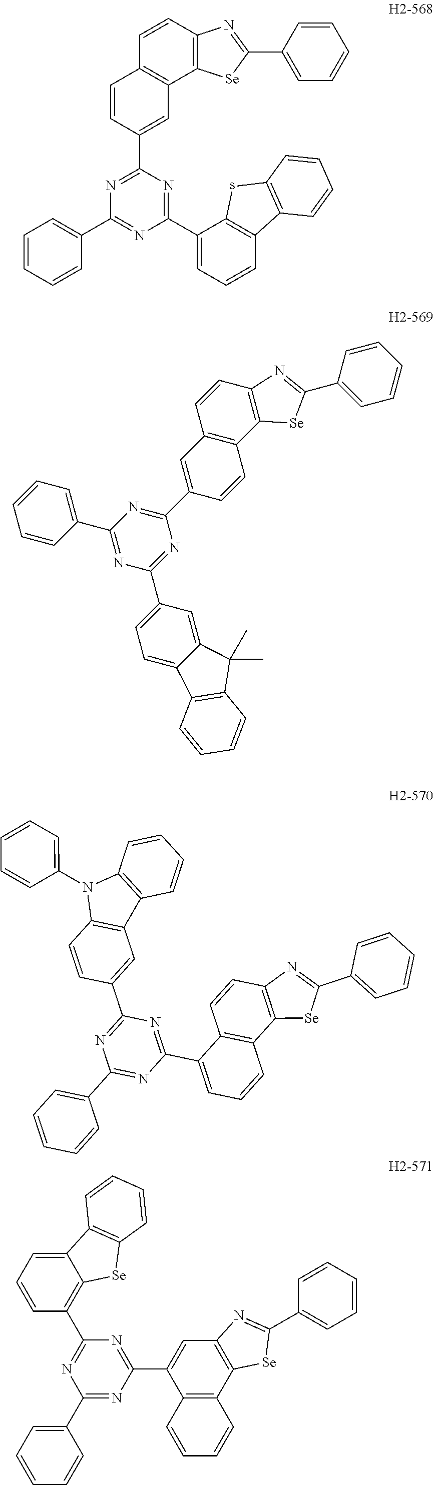

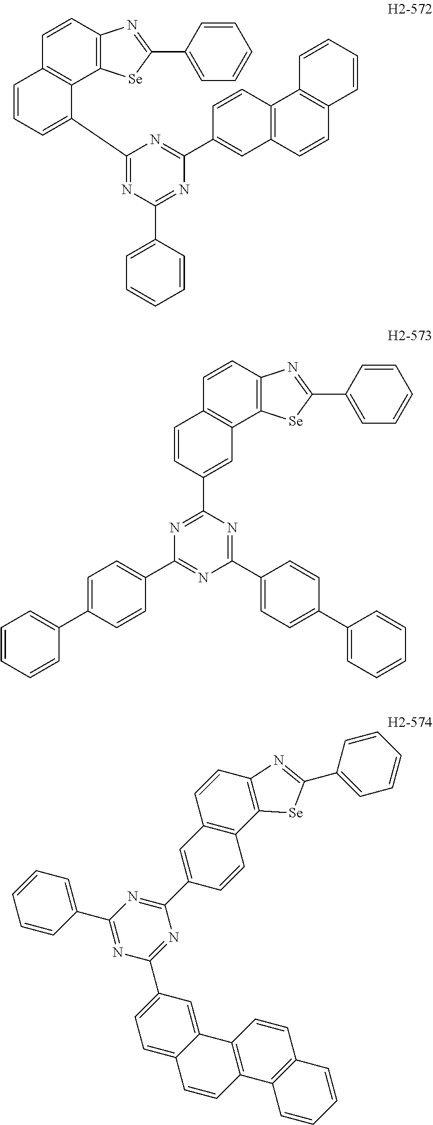 [SMILES: c1ccc(-c2nc(-c3ccc4c(ccc5ccccc54)c3)nc(-c3cccc4ccc5nc(-c6ccccc6)[se]c5c34)n2)cc1 ; c1ccc(-c2ccc(-c3nc(-c4ccc(-c5ccccc5)cc4)nc(-c4ccc5ccc6nc(-c7ccccc7)[se]c6c5c4)n3)cc2)cc1 ; c1ccc(-c2nc(-c3ccc4c(ccc5nc(-c6ccccc6)[se]c54)c3)nc(-c3ccc4ccc5c6ccccc6ccc5c4c3)n2)cc1]

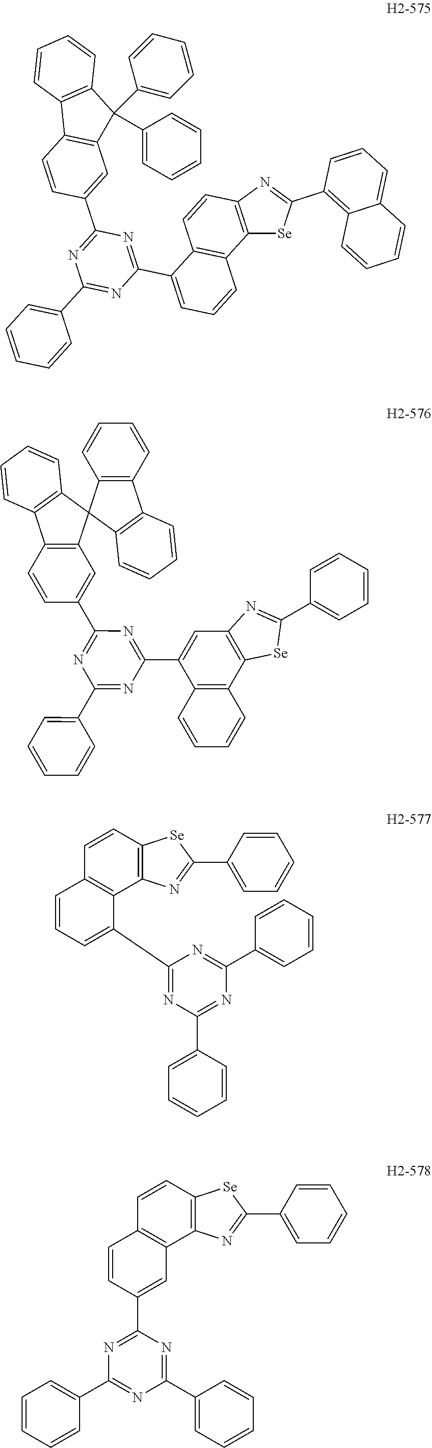 [SMILES: c1ccc(-c2nc(-c3ccc4c(c3)C(c3ccccc3)(c3ccccc3)c3ccccc3-4)nc(-c3cccc4c3ccc3nc(-c5cccc6ccccc56)[se]c34)n2)cc1 ; c1ccc(-c2nc(-c3ccc4c(c3)C3(c5ccccc5-c5ccccc53)c3ccccc3-4)nc(-c3cc4nc(-c5ccccc5)[se]c4c4ccccc34)n2)cc1 ; c1ccc(-c2nc(-c3ccccc3)nc(-c3cccc4ccc5[se]c(-c6ccccc6)nc5c34)n2)cc1 ; c1ccc(-c2nc(-c3ccccc3)nc(-c3ccc4ccc5[se]c(-c6ccccc6)nc5c4c3)n2)cc1]

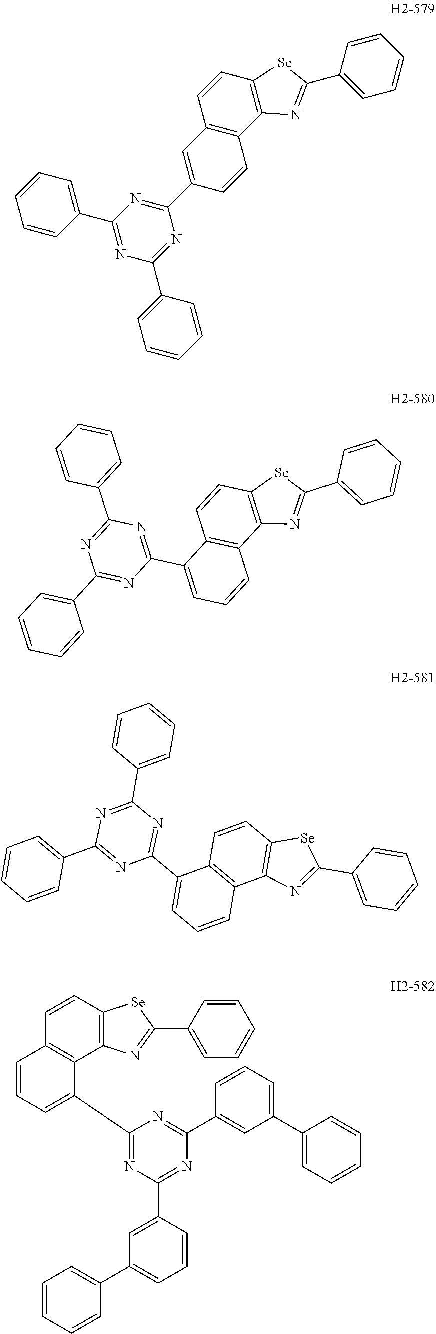 [SMILES: c1ccc(-c2nc(-c3ccccc3)nc(-c3ccc4c(ccc5[se]c(-c6ccccc6)nc54)c3)n2)cc1 ; c1ccc(-c2nc(-c3ccccc3)nc(-c3cccc4c3ccc3[se]c(-c5ccccc5)nc34)n2)cc1 ; c1ccc(-c2nc(-c3ccccc3)nc(-c3cccc4c3ccc3[se]c(-c5ccccc5)nc34)n2)cc1 ; c1ccc(-c2cccc(-c3nc(-c4cccc(-c5ccccc5)c4)nc(-c4cccc5ccc6[se]c(-c7ccccc7)nc6c45)n3)c2)cc1]

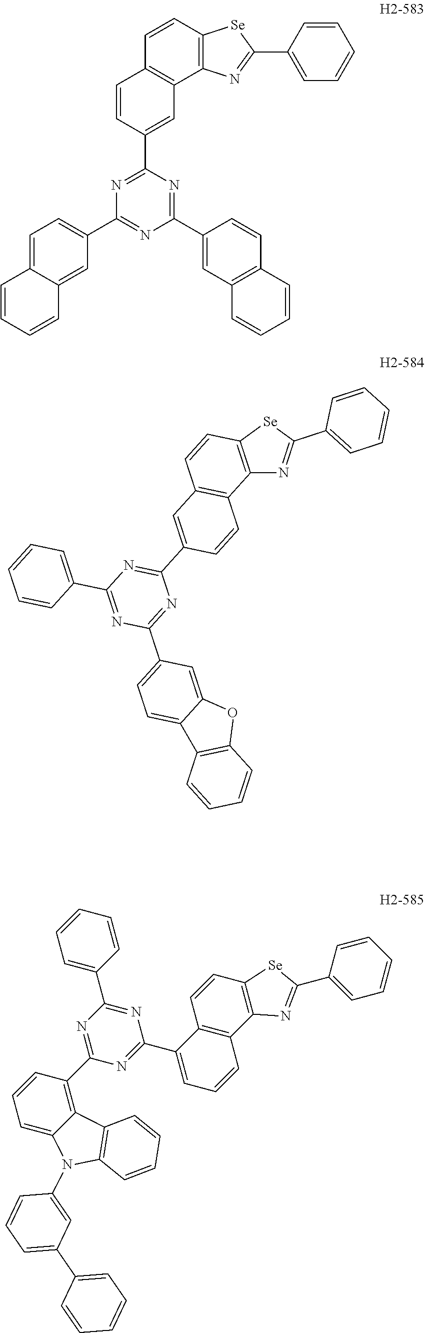 [SMILES: c1ccc(-c2nc3c(ccc4ccc(-c5nc(-c6ccc7ccccc7c6)nc(-c6ccc7ccccc7c6)n5)cc43)[se]2)cc1 ; c1ccc(-c2nc(-c3ccc4c(ccc5[se]c(-c6ccccc6)nc54)c3)nc(-c3ccc4c(c3)oc3ccccc34)n2)cc1 ; c1ccc(-c2cccc(-n3c4ccccc4c4c(-c5nc(-c6ccccc6)nc(-c6cccc7c6ccc6[se]c(-c8ccccc8)nc67)n5)cccc43)c2)cc1]

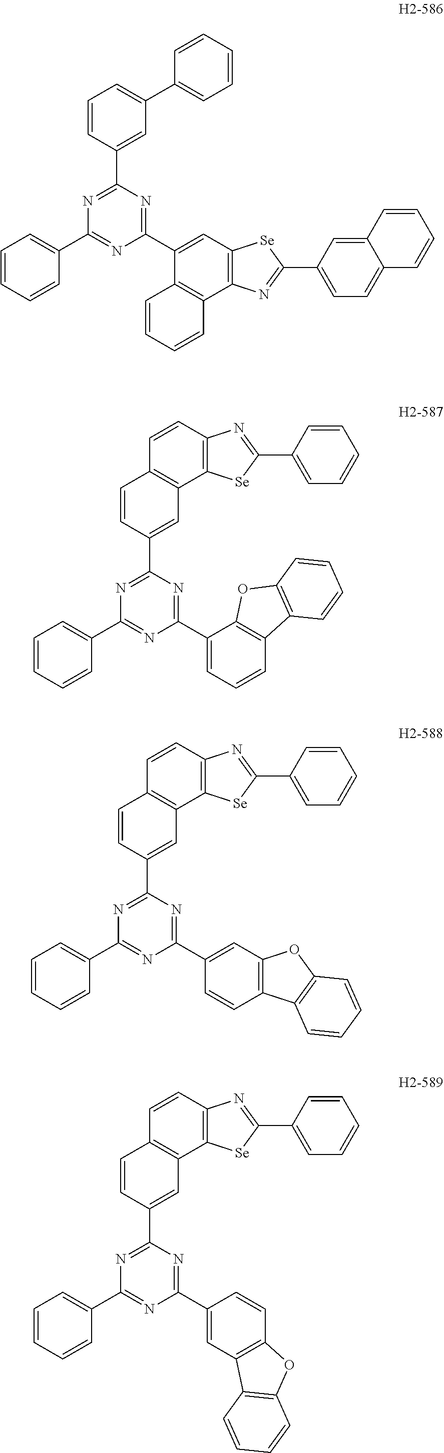 [SMILES: c1ccc(-c2cccc(-c3nc(-c4ccccc4)nc(-c4cc5[se]c(-c6ccc7ccccc7c6)nc5c5ccccc45)n3)c2)cc1 ; c1ccc(-c2nc(-c3ccc4ccc5nc(-c6ccccc6)[se]c5c4c3)nc(-c3cccc4c3oc3ccccc34)n2)cc1 ; c1ccc(-c2nc(-c3ccc4c(c3)oc3ccccc34)nc(-c3ccc4ccc5nc(-c6ccccc6)[se]c5c4c3)n2)cc1 ; c1ccc(-c2nc(-c3ccc4oc5ccccc5c4c3)nc(-c3ccc4ccc5nc(-c6ccccc6)[se]c5c4c3)n2)cc1]

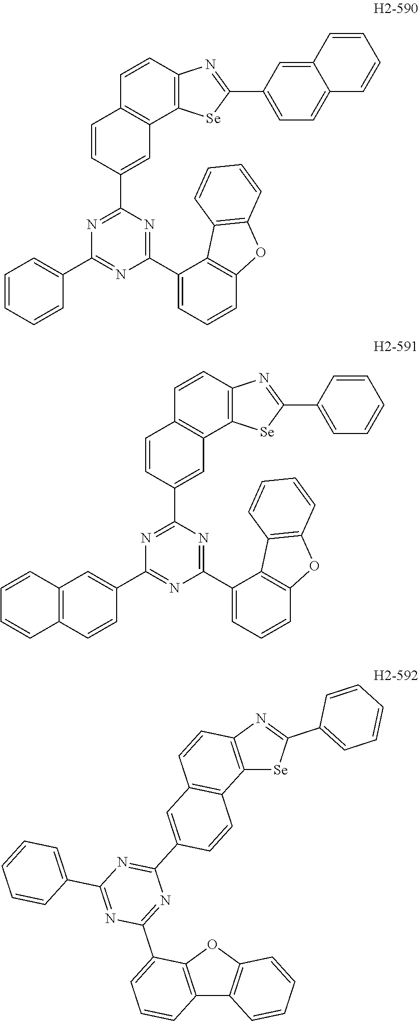 [SMILES: c1ccc(-c2nc(-c3ccc4ccc5nc(-c6ccc7ccccc7c6)[se]c5c4c3)nc(-c3cccc4oc5ccccc5c34)n2)cc1 ; c1ccc(-c2nc3ccc4ccc(-c5nc(-c6ccc7ccccc7c6)nc(-c6cccc7oc8ccccc8c67)n5)cc4c3[se]2)cc1 ; c1ccc(-c2nc(-c3ccc4c(ccc5nc(-c6ccccc6)[se]c54)c3)nc(-c3cccc4c3oc3ccccc34)n2)cc1]

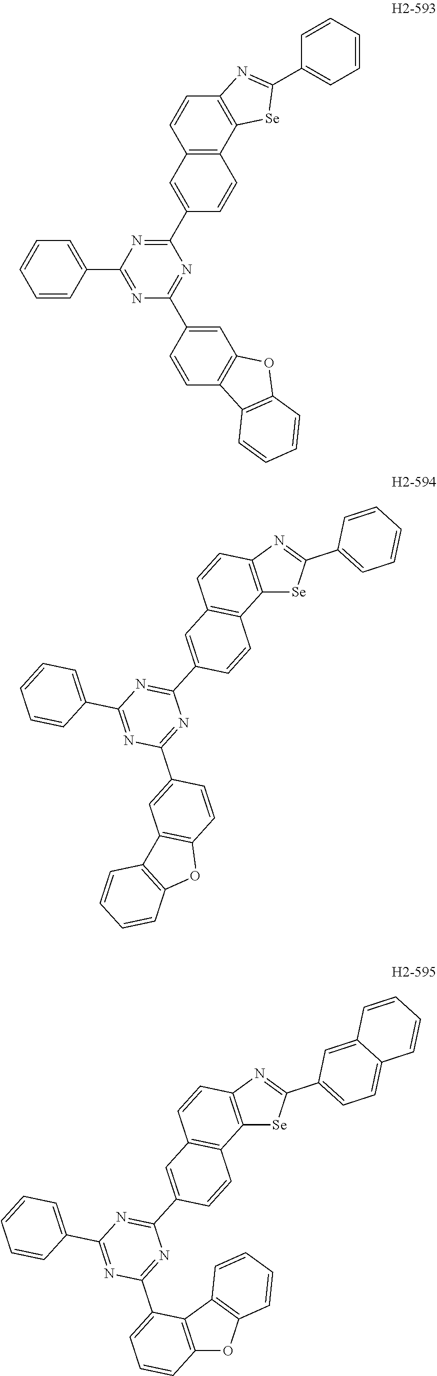 [SMILES: c1ccc(-c2nc(-c3ccc4c(ccc5nc(-c6ccccc6)[se]c54)c3)nc(-c3ccc4c(c3)oc3ccccc34)n2)cc1 ; c1ccc(-c2nc(-c3ccc4c(ccc5nc(-c6ccccc6)[se]c54)c3)nc(-c3ccc4oc5ccccc5c4c3)n2)cc1 ; c1ccc(-c2nc(-c3ccc4c(ccc5nc(-c6ccc7ccccc7c6)[se]c54)c3)nc(-c3cccc4oc5ccccc5c34)n2)cc1]

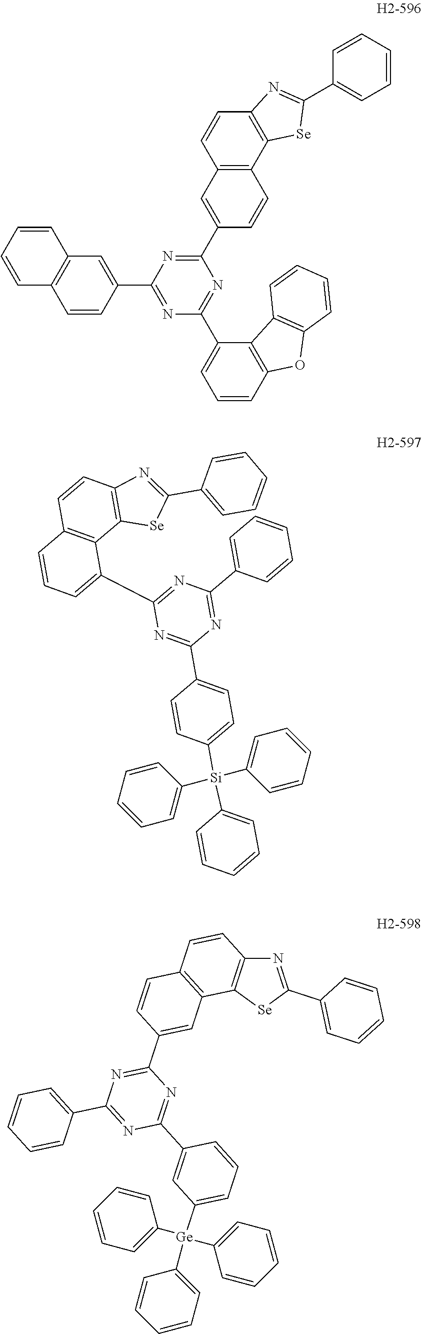

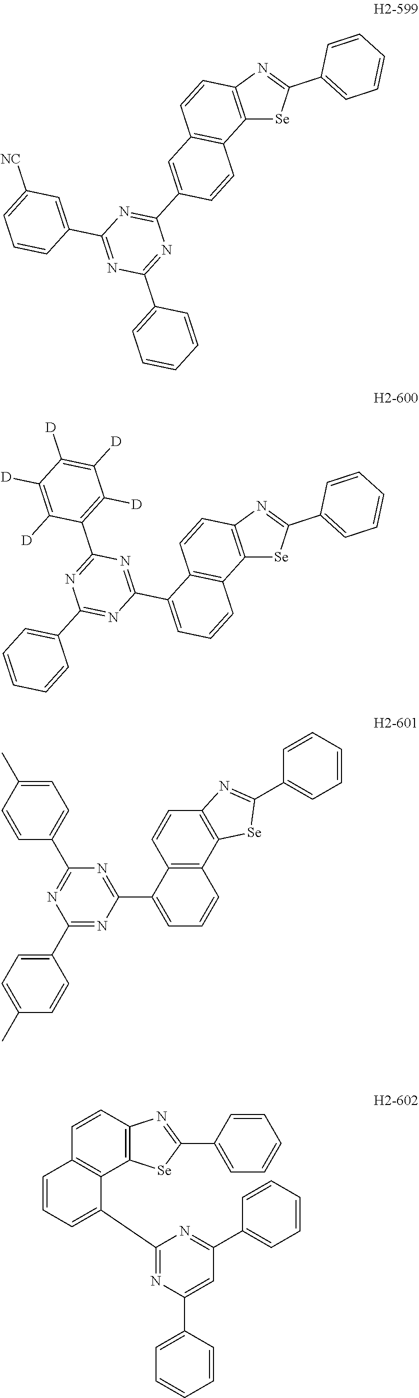 [SMILES: N#Cc1cccc(-c2nc(-c3ccccc3)nc(-c3ccc4c(ccc5nc(-c6ccccc6)[se]c54)c3)n2)c1 ; [2H]c1c([2H])c([2H])c(-c2nc(-c3ccccc3)nc(-c3cccc4c3ccc3nc(-c5ccccc5)[se]c34)n2)c([2H])c1[2H] ; Cc1ccc(-c2nc(-c3ccc(C)cc3)nc(-c3cccc4c3ccc3nc(-c5ccccc5)[se]c34)n2)cc1 ; c1ccc(-c2cc(-c3ccccc3)nc(-c3cccc4ccc5nc(-c6ccccc6)[se]c5c34)n2)cc1]

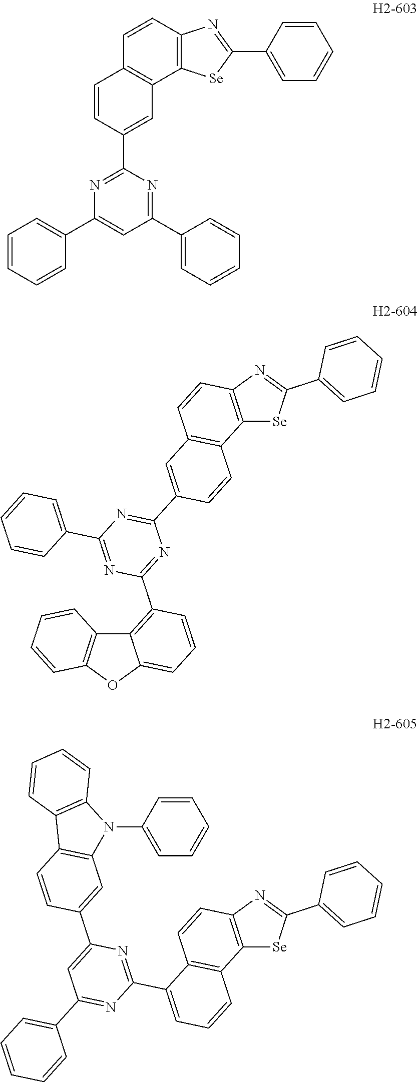 [SMILES: c1ccc(-c2cc(-c3ccccc3)nc(-c3ccc4ccc5nc(-c6ccccc6)[se]c5c4c3)n2)cc1 ; c1ccc(-c2nc(-c3ccc4c(ccc5nc(-c6ccccc6)[se]c54)c3)nc(-c3cccc4oc5ccccc5c34)n2)cc1 ; c1ccc(-c2cc(-c3ccc4c5ccccc5n(-c5ccccc5)c4c3)nc(-c3cccc4c3ccc3nc(-c5ccccc5)[se]c34)n2)cc1]

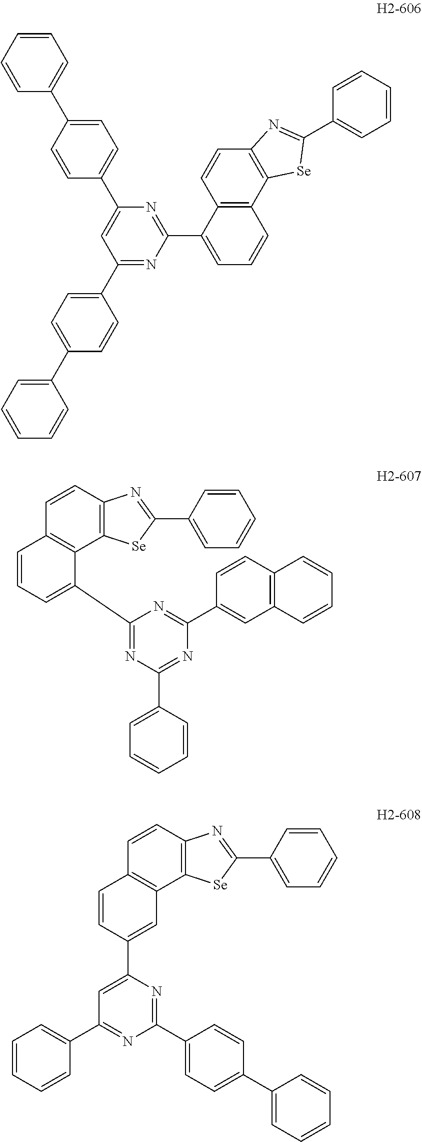 [SMILES: c1ccc(-c2ccc(-c3cc(-c4ccc(-c5ccccc5)cc4)nc(-c4cccc5c4ccc4nc(-c6ccccc6)[se]c45)n3)cc2)cc1 ; c1ccc(-c2nc(-c3ccc4ccccc4c3)nc(-c3cccc4ccc5nc(-c6ccccc6)[se]c5c34)n2)cc1 ; c1ccc(-c2ccc(-c3nc(-c4ccccc4)cc(-c4ccc5ccc6nc(-c7ccccc7)[se]c6c5c4)n3)cc2)cc1]

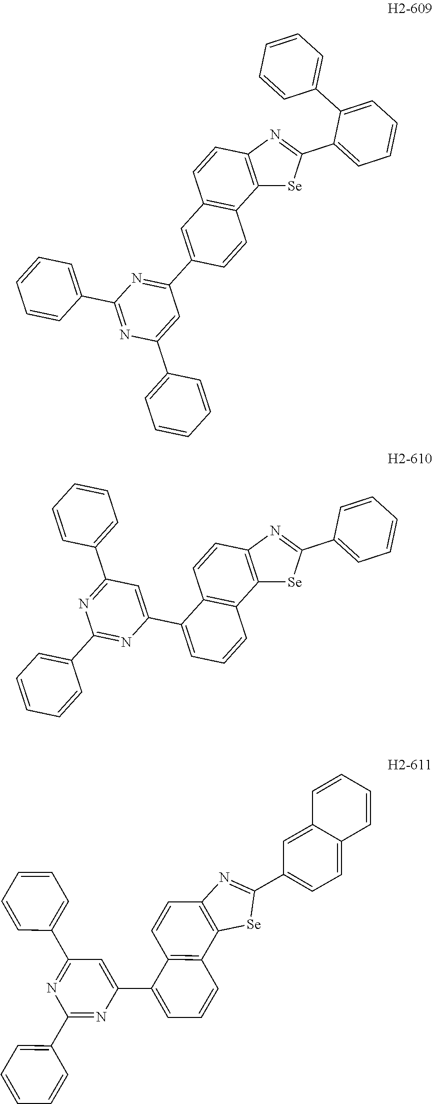 [SMILES: c1ccc(-c2cc(-c3ccc4c(ccc5nc(-c6ccccc6-c6ccccc6)[se]c54)c3)nc(-c3ccccc3)n2)cc1 ; c1ccc(-c2cc(-c3cccc4c3ccc3nc(-c5ccccc5)[se]c34)nc(-c3ccccc3)n2)cc1 ; c1ccc(-c2cc(-c3cccc4c3ccc3nc(-c5ccc6ccccc6c5)[se]c34)nc(-c3ccccc3)n2)cc1]

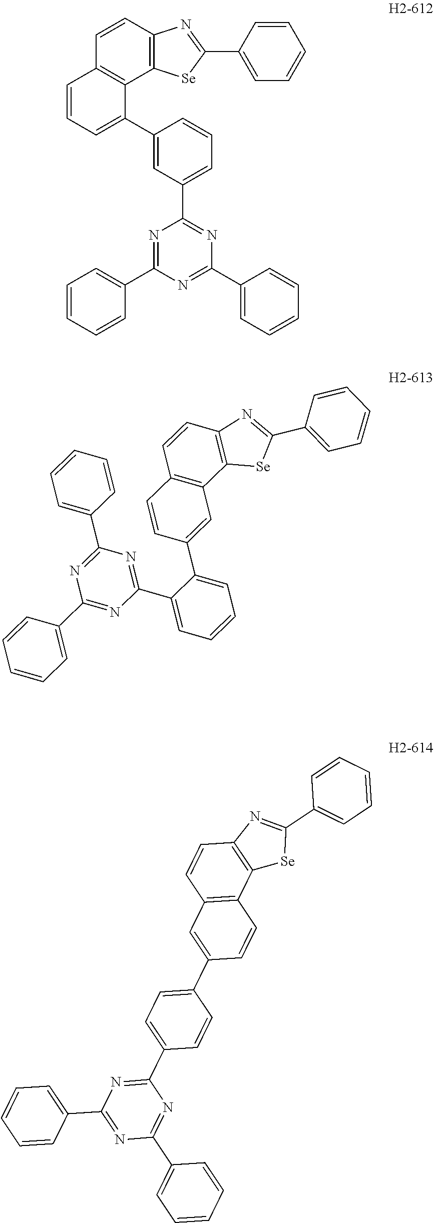 [SMILES: c1ccc(-c2nc(-c3ccccc3)nc(-c3cccc(-c4cccc5ccc6nc(-c7ccccc7)[se]c6c45)c3)n2)cc1 ; c1ccc(-c2nc(-c3ccccc3)nc(-c3ccccc3-c3ccc4ccc5nc(-c6ccccc6)[se]c5c4c3)n2)cc1 ; c1ccc(-c2nc(-c3ccccc3)nc(-c3ccc(-c4ccc5c(ccc6nc(-c7ccccc7)[se]c65)c4)cc3)n2)cc1]

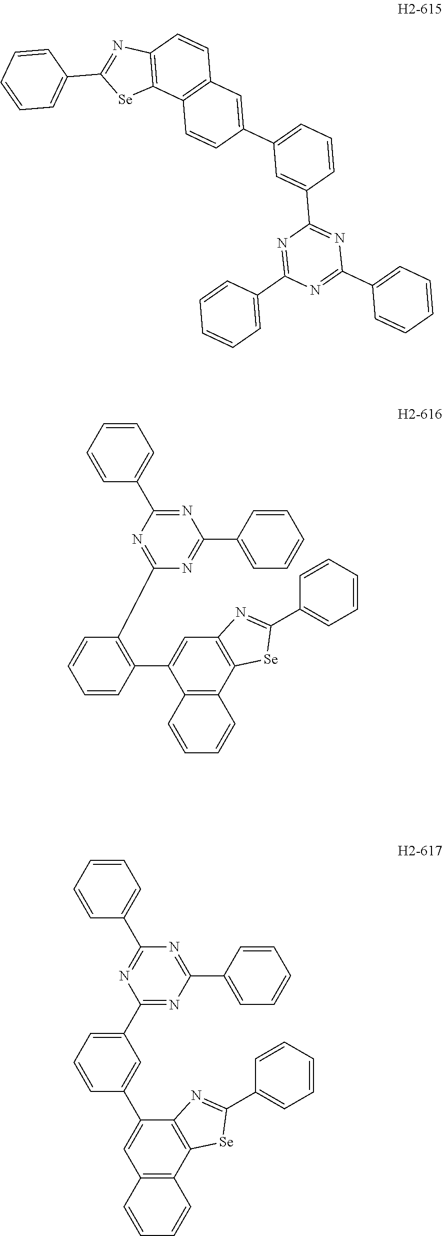 [SMILES: c1ccc(-c2nc(-c3ccccc3)nc(-c3cccc(-c4ccc5c(ccc6nc(-c7ccccc7)[se]c65)c4)c3)n2)cc1 ; c1ccc(-c2nc(-c3ccccc3)nc(-c3ccccc3-c3cc4nc(-c5ccccc5)[se]c4c4ccccc34)n2)cc1 ; c1ccc(-c2nc(-c3ccccc3)nc(-c3cccc(-c4cc5ccccc5c5[se]c(-c6ccccc6)nc45)c3)n2)cc1]

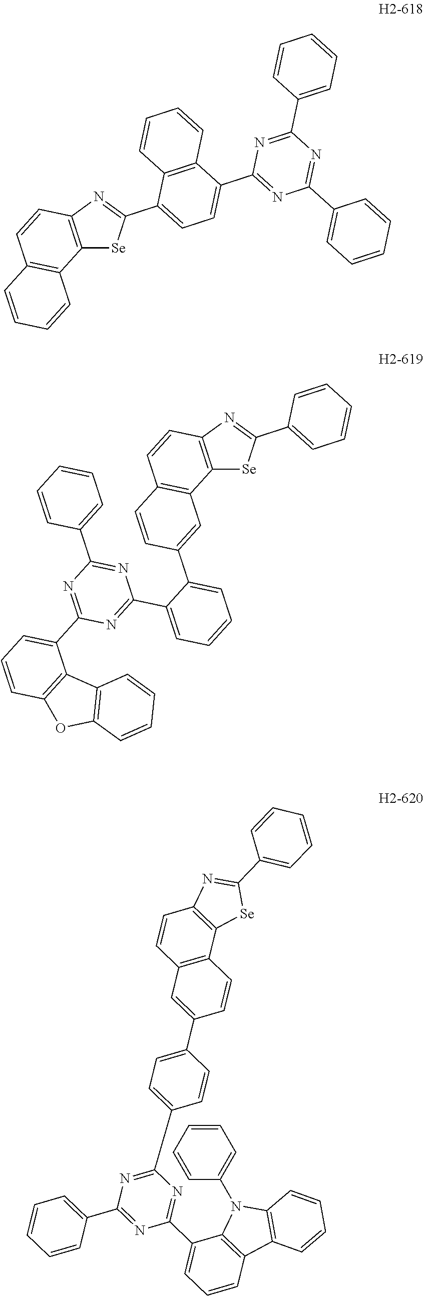 [SMILES: c1ccc(-c2nc(-c3ccccc3)nc(-c3ccc(-c4nc5ccc6ccccc6c5[se]4)c4ccccc34)n2)cc1 ; c1ccc(-c2nc(-c3ccccc3-c3ccc4ccc5nc(-c6ccccc6)[se]c5c4c3)nc(-c3cccc4oc5ccccc5c34)n2)cc1 ; c1ccc(-c2nc(-c3ccc(-c4ccc5c(ccc6nc(-c7ccccc7)[se]c65)c4)cc3)nc(-c3cccc4c5ccccc5n(-c5ccccc5)c34)n2)cc1]

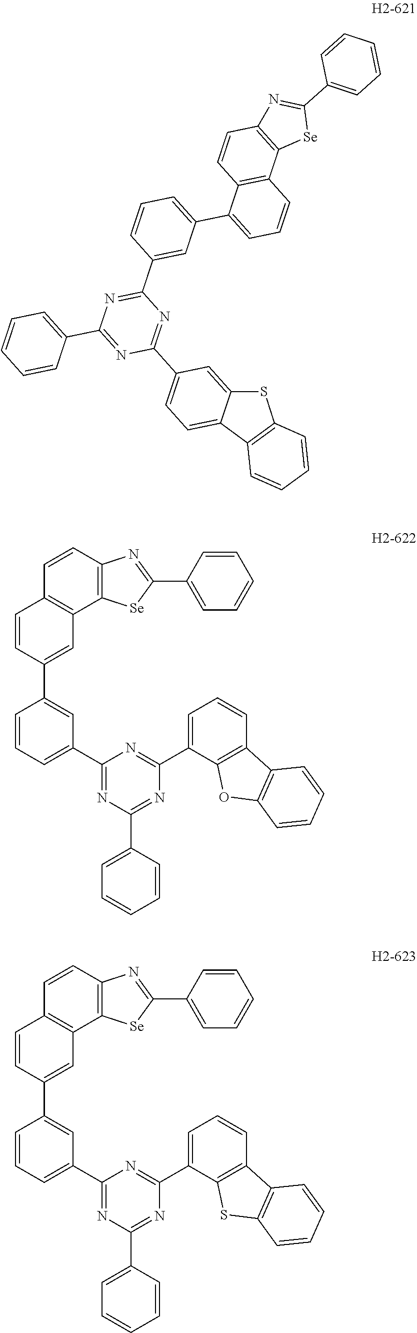 [SMILES: c1ccc(-c2nc(-c3cccc(-c4cccc5c4ccc4nc(-c6ccccc6)[se]c45)c3)nc(-c3ccc4c(c3)sc3ccccc34)n2)cc1 ; c1ccc(-c2nc(-c3cccc(-c4ccc5ccc6nc(-c7ccccc7)[se]c6c5c4)c3)nc(-c3cccc4c3oc3ccccc34)n2)cc1 ; c1ccc(-c2nc(-c3cccc(-c4ccc5ccc6nc(-c7ccccc7)[se]c6c5c4)c3)nc(-c3cccc4c3sc3ccccc34)n2)cc1]

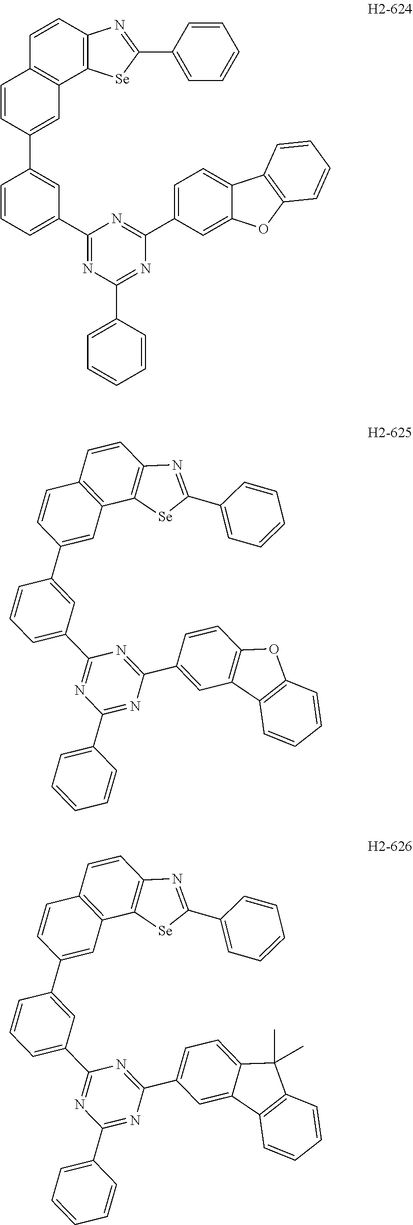 [SMILES: c1ccc(-c2nc(-c3cccc(-c4ccc5ccc6nc(-c7ccccc7)[se]c6c5c4)c3)nc(-c3ccc4c(c3)oc3ccccc34)n2)cc1 ; c1ccc(-c2nc(-c3cccc(-c4ccc5ccc6nc(-c7ccccc7)[se]c6c5c4)c3)nc(-c3ccc4oc5ccccc5c4c3)n2)cc1 ; CC1(C)c2ccccc2-c2cc(-c3nc(-c4ccccc4)nc(-c4cccc(-c5ccc6ccc7nc(-c8ccccc8)[se]c7c6c5)c4)n3)ccc21]

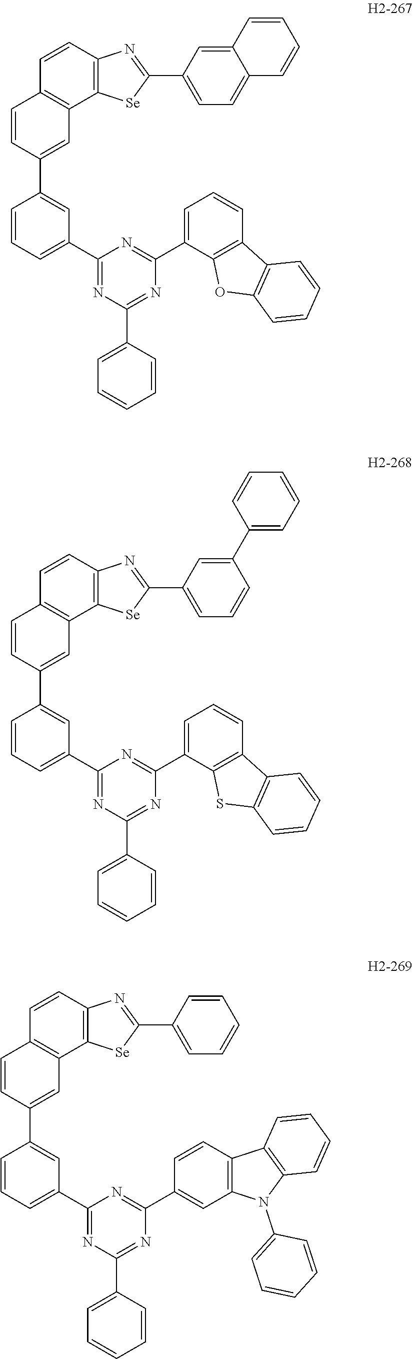 [SMILES: c1ccc(-c2nc(-c3cccc(-c4ccc5ccc6nc(-c7ccc8ccccc8c7)[se]c6c5c4)c3)nc(-c3cccc4c3oc3ccccc34)n2)cc1 ; c1ccc(-c2cccc(-c3nc4ccc5ccc(-c6cccc(-c7nc(-c8ccccc8)nc(-c8cccc9c8sc8ccccc89)n7)c6)cc5c4[se]3)c2)cc1 ; c1ccc(-c2nc(-c3cccc(-c4ccc5ccc6nc(-c7ccccc7)[se]c6c5c4)c3)nc(-c3ccc4c5ccccc5n(-c5ccccc5)c4c3)n2)cc1]

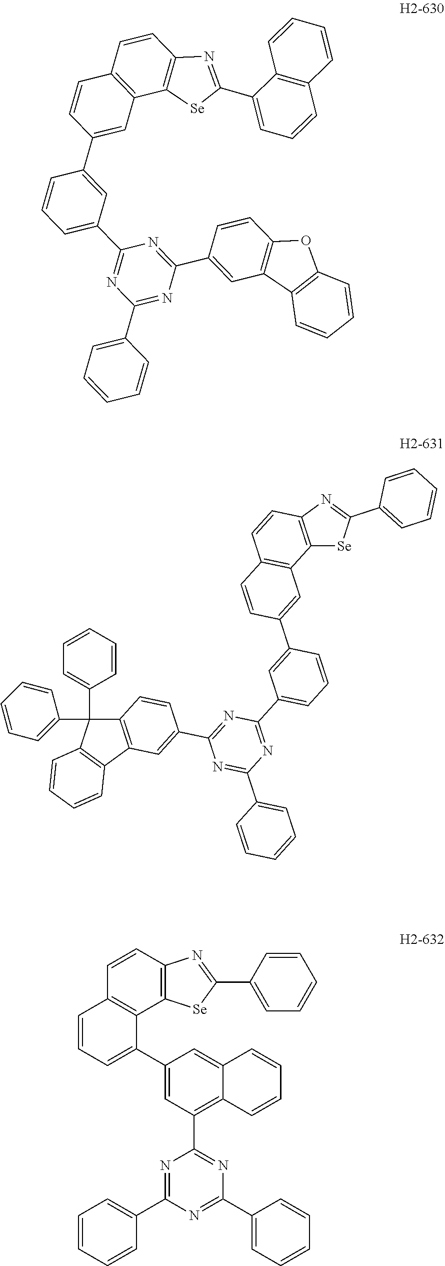 [SMILES: c1ccc(-c2nc(-c3cccc(-c4ccc5ccc6nc(-c7cccc8ccccc78)[se]c6c5c4)c3)nc(-c3ccc4oc5ccccc5c4c3)n2)cc1 ; c1ccc(-c2nc(-c3cccc(-c4ccc5ccc6nc(-c7ccccc7)[se]c6c5c4)c3)nc(-c3ccc4c(c3)-c3ccccc3C4(c3ccccc3)c3ccccc3)n2)cc1 ; c1ccc(-c2nc(-c3ccccc3)nc(-c3cc(-c4cccc5ccc6nc(-c7ccccc7)[se]c6c45)cc4ccccc34)n2)cc1]

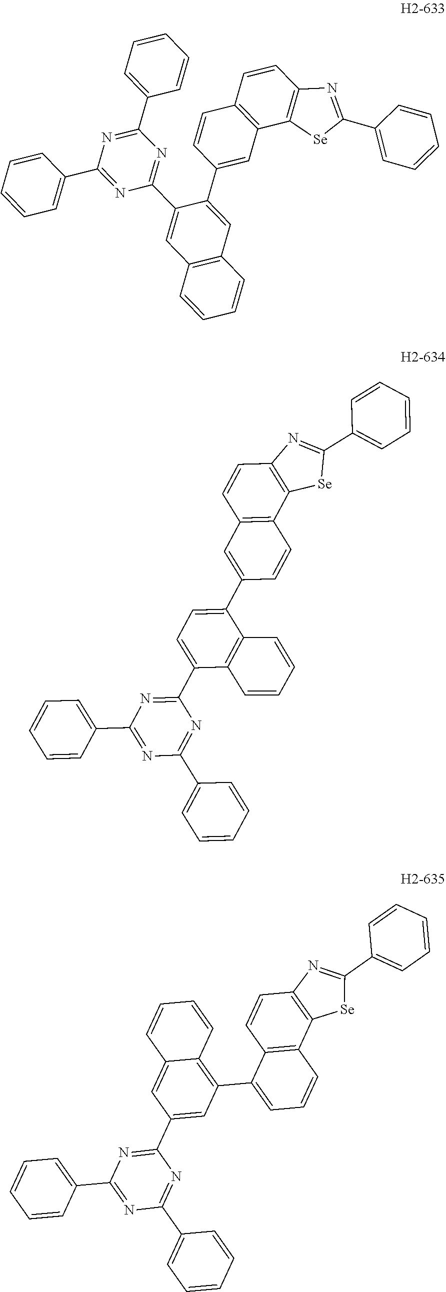 [SMILES: c1ccc(-c2nc(-c3ccccc3)nc(-c3cc4ccccc4cc3-c3ccc4ccc5nc(-c6ccccc6)[se]c5c4c3)n2)cc1 ; c1ccc(-c2nc(-c3ccccc3)nc(-c3ccc(-c4ccc5c(ccc6nc(-c7ccccc7)[se]c65)c4)c4ccccc34)n2)cc1 ; c1ccc(-c2nc(-c3ccccc3)nc(-c3cc(-c4cccc5c4ccc4nc(-c6ccccc6)[se]c45)c4ccccc4c3)n2)cc1]

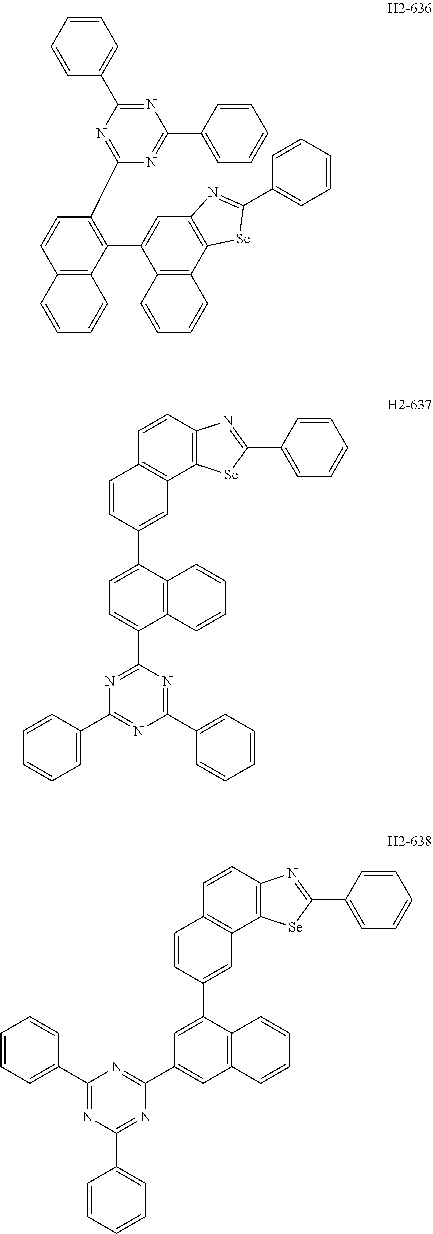 [SMILES: c1ccc(-c2nc(-c3ccccc3)nc(-c3ccc4ccccc4c3-c3cc4nc(-c5ccccc5)[se]c4c4ccccc34)n2)cc1 ; c1ccc(-c2nc(-c3ccccc3)nc(-c3ccc(-c4ccc5ccc6nc(-c7ccccc7)[se]c6c5c4)c4ccccc34)n2)cc1 ; c1ccc(-c2nc(-c3ccccc3)nc(-c3cc(-c4ccc5ccc6nc(-c7ccccc7)[se]c6c5c4)c4ccccc4c3)n2)cc1]

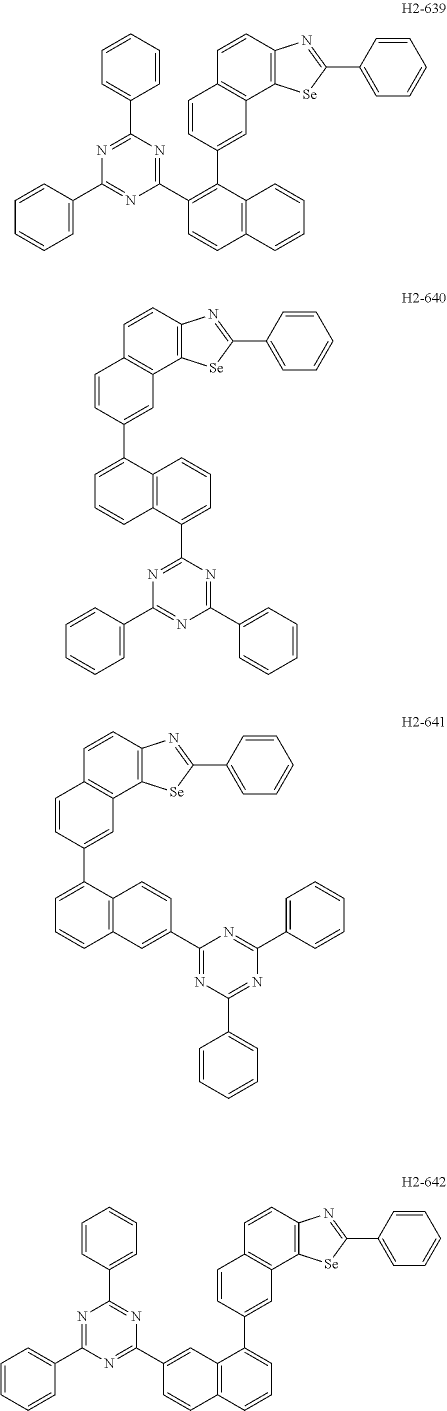 [SMILES: c1ccc(-c2nc(-c3ccccc3)nc(-c3ccc4ccccc4c3-c3ccc4ccc5nc(-c6ccccc6)[se]c5c4c3)n2)cc1 ; c1ccc(-c2nc(-c3ccccc3)nc(-c3cccc4c(-c5ccc6ccc7nc(-c8ccccc8)[se]c7c6c5)cccc34)n2)cc1 ; c1ccc(-c2nc(-c3ccccc3)nc(-c3ccc4c(-c5ccc6ccc7nc(-c8ccccc8)[se]c7c6c5)cccc4c3)n2)cc1 ; c1ccc(-c2nc(-c3ccccc3)nc(-c3ccc4cccc(-c5ccc6ccc7nc(-c8ccccc8)[se]c7c6c5)c4c3)n2)cc1]

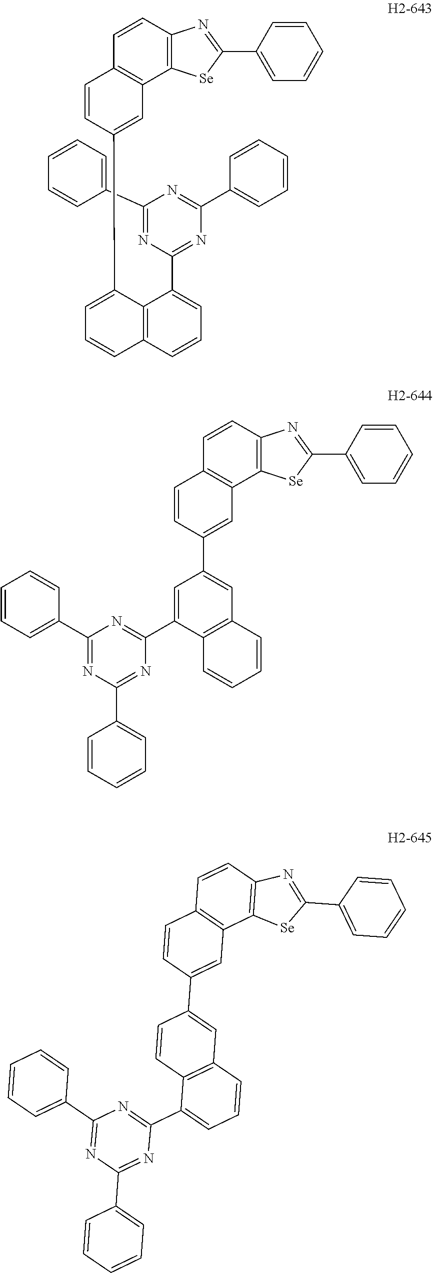 [SMILES: c1ccc(-c2nc(-c3ccccc3)nc(-c3cccc4cccc(-c5ccc6ccc7nc(-c8ccccc8)[se]c7c6c5)c34)n2)cc1 ; c1ccc(-c2nc(-c3ccccc3)nc(-c3cc(-c4ccc5ccc6nc(-c7ccccc7)[se]c6c5c4)cc4ccccc34)n2)cc1 ; c1ccc(-c2nc(-c3ccccc3)nc(-c3cccc4cc(-c5ccc6ccc7nc(-c8ccccc8)[se]c7c6c5)ccc34)n2)cc1]

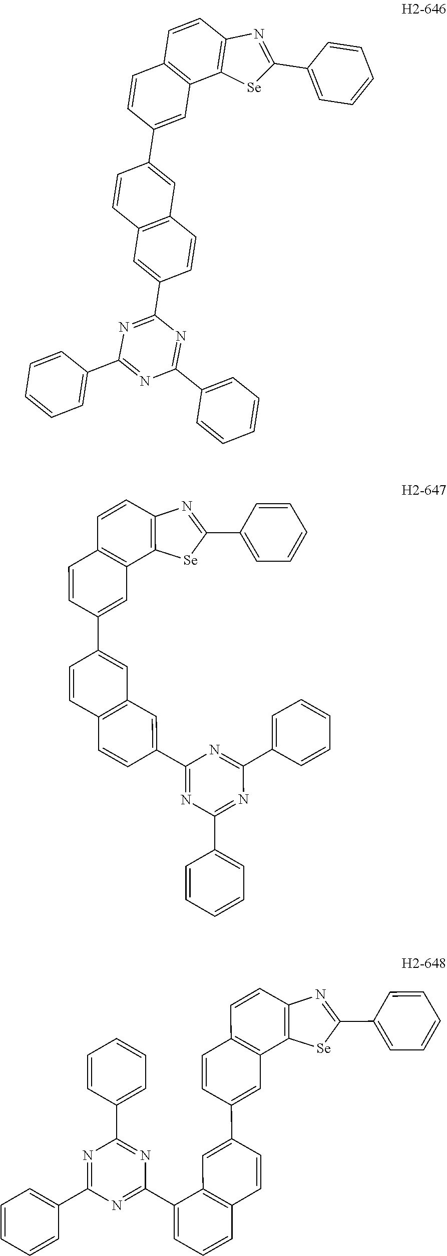 [SMILES: c1ccc(-c2nc(-c3ccccc3)nc(-c3ccc4cc(-c5ccc6ccc7nc(-c8ccccc8)[se]c7c6c5)ccc4c3)n2)cc1 ; c1ccc(-c2nc(-c3ccccc3)nc(-c3ccc4ccc(-c5ccc6ccc7nc(-c8ccccc8)[se]c7c6c5)cc4c3)n2)cc1 ; c1ccc(-c2nc(-c3ccccc3)nc(-c3cccc4ccc(-c5ccc6ccc7nc(-c8ccccc8)[se]c7c6c5)cc34)n2)cc1]

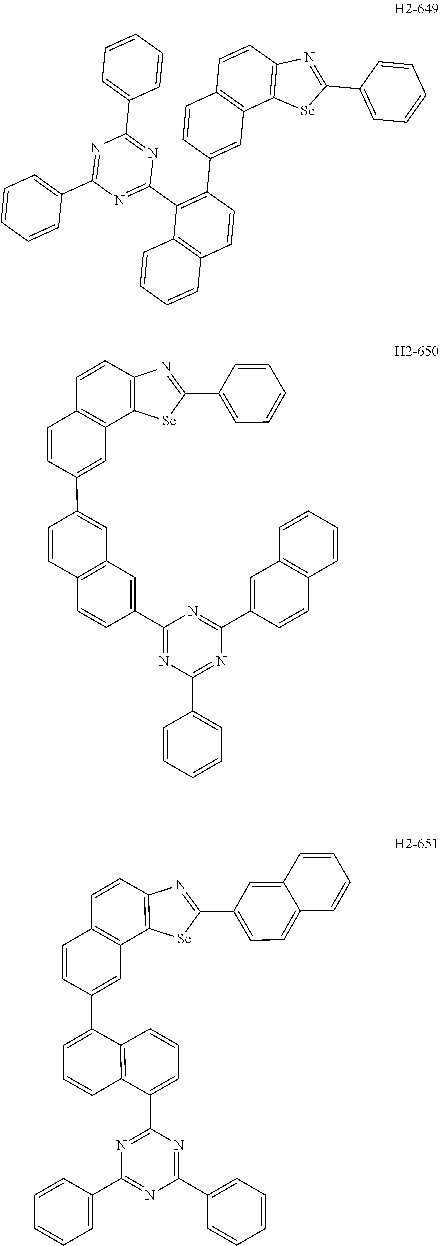 [SMILES: c1ccc(-c2nc(-c3ccccc3)nc(-c3c(-c4ccc5ccc6nc(-c7ccccc7)[se]c6c5c4)ccc4ccccc34)n2)cc1 ; c1ccc(-c2nc(-c3ccc4ccccc4c3)nc(-c3ccc4ccc(-c5ccc6ccc7nc(-c8ccccc8)[se]c7c6c5)cc4c3)n2)cc1 ; c1ccc(-c2nc(-c3ccccc3)nc(-c3cccc4c(-c5ccc6ccc7nc(-c8ccc9ccccc9c8)[se]c7c6c5)cccc34)n2)cc1]

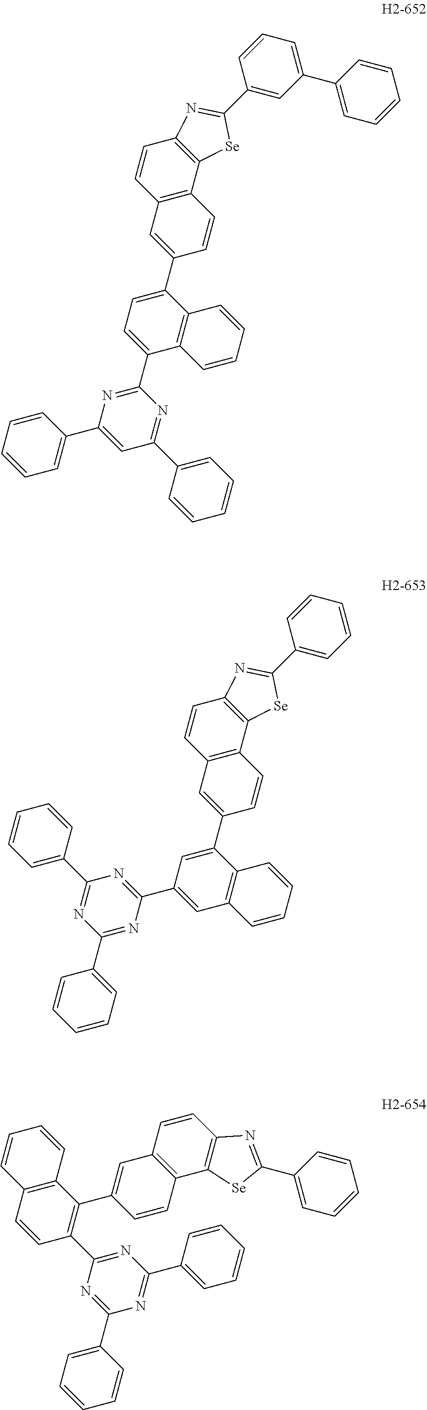 [SMILES: c1ccc(-c2cccc(-c3nc4ccc5cc(-c6ccc(-c7nc(-c8ccccc8)cc(-c8ccccc8)n7)c7ccccc67)ccc5c4[se]3)c2)cc1 ; c1ccc(-c2nc(-c3ccccc3)nc(-c3cc(-c4ccc5c(ccc6nc(-c7ccccc7)[se]c65)c4)c4ccccc4c3)n2)cc1 ; c1ccc(-c2nc(-c3ccccc3)nc(-c3ccc4ccccc4c3-c3ccc4c(ccc5nc(-c6ccccc6)[se]c54)c3)n2)cc1]

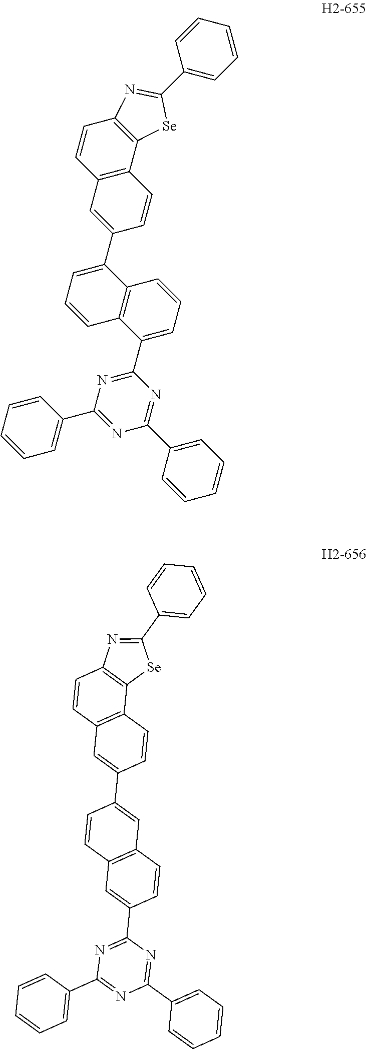 [SMILES: c1ccc(-c2nc(-c3ccccc3)nc(-c3cccc4c(-c5ccc6c(ccc7nc(-c8ccccc8)[se]c76)c5)cccc34)n2)cc1 ; c1ccc(-c2nc(-c3ccccc3)nc(-c3ccc4cc(-c5ccc6c(ccc7nc(-c8ccccc8)[se]c76)c5)ccc4c3)n2)cc1]

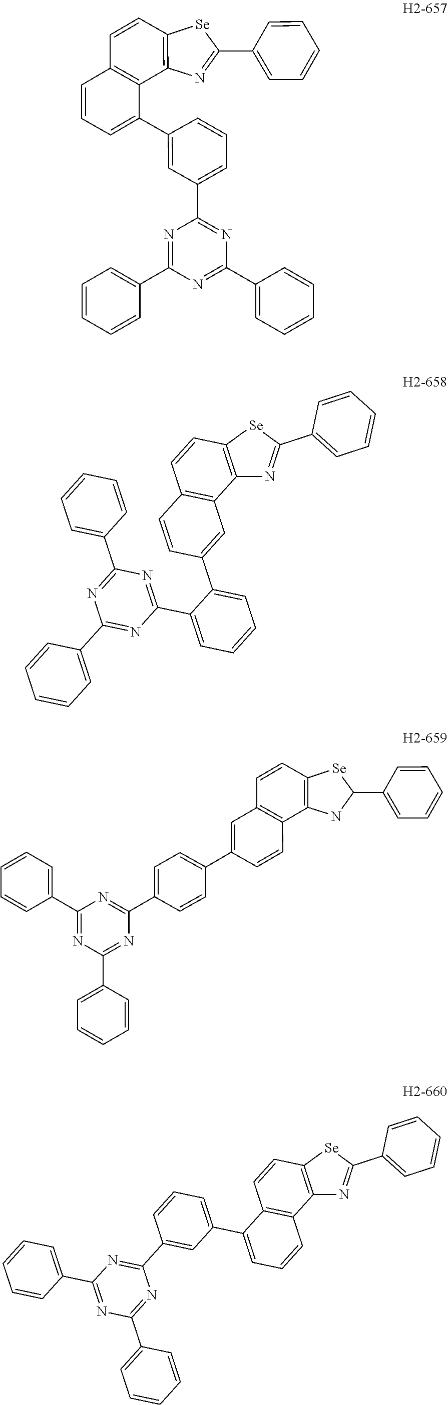 [SMILES: c1ccc(-c2nc(-c3ccccc3)nc(-c3cccc(-c4cccc5ccc6[se]c(-c7ccccc7)nc6c45)c3)n2)cc1 ; c1ccc(-c2nc(-c3ccccc3)nc(-c3ccccc3-c3ccc4ccc5[se]c(-c6ccccc6)nc5c4c3)n2)cc1 ; c1ccc(-c2nc(-c3ccccc3)nc(-c3ccc(-c4ccc5c6c(ccc5c4)[Se]C(c4ccccc4)N6)cc3)n2)cc1 ; c1ccc(-c2nc(-c3ccccc3)nc(-c3cccc(-c4cccc5c4ccc4[se]c(-c6ccccc6)nc45)c3)n2)cc1]

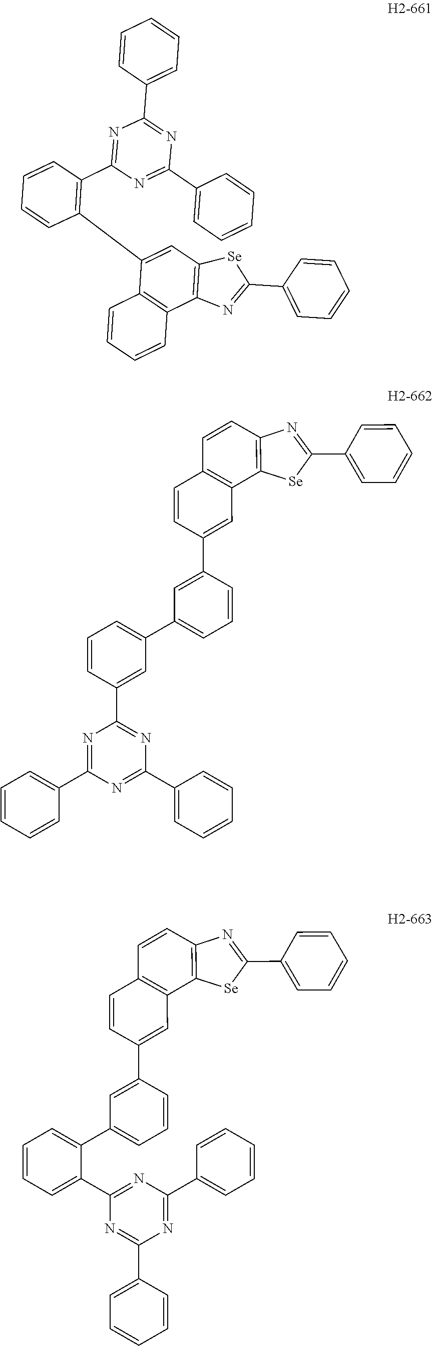 [SMILES: c1ccc(-c2nc(-c3ccccc3)nc(-c3ccccc3-c3cc4[se]c(-c5ccccc5)nc4c4ccccc34)n2)cc1 ; c1ccc(-c2nc(-c3ccccc3)nc(-c3cccc(-c4cccc(-c5ccc6ccc7nc(-c8ccccc8)[se]c7c6c5)c4)c3)n2)cc1 ; c1ccc(-c2nc(-c3ccccc3)nc(-c3ccccc3-c3cccc(-c4ccc5ccc6nc(-c7ccccc7)[se]c6c5c4)c3)n2)cc1]

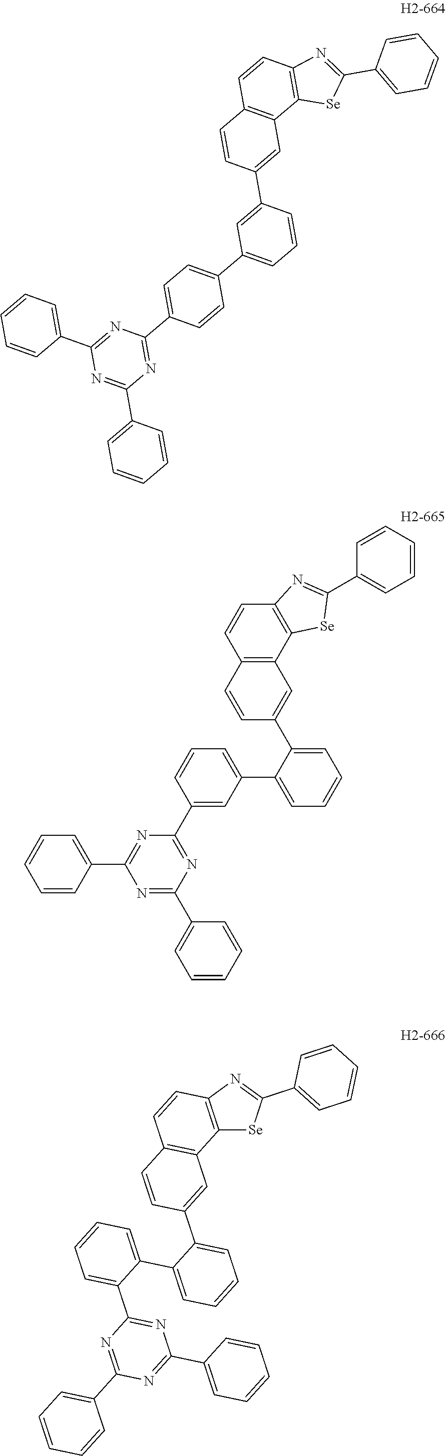 [SMILES: c1ccc(-c2nc(-c3ccccc3)nc(-c3ccc(-c4cccc(-c5ccc6ccc7nc(-c8ccccc8)[se]c7c6c5)c4)cc3)n2)cc1 ; c1ccc(-c2nc(-c3ccccc3)nc(-c3cccc(-c4ccccc4-c4ccc5ccc6nc(-c7ccccc7)[se]c6c5c4)c3)n2)cc1 ; c1ccc(-c2nc(-c3ccccc3)nc(-c3ccccc3-c3ccccc3-c3ccc4ccc5nc(-c6ccccc6)[se]c5c4c3)n2)cc1]

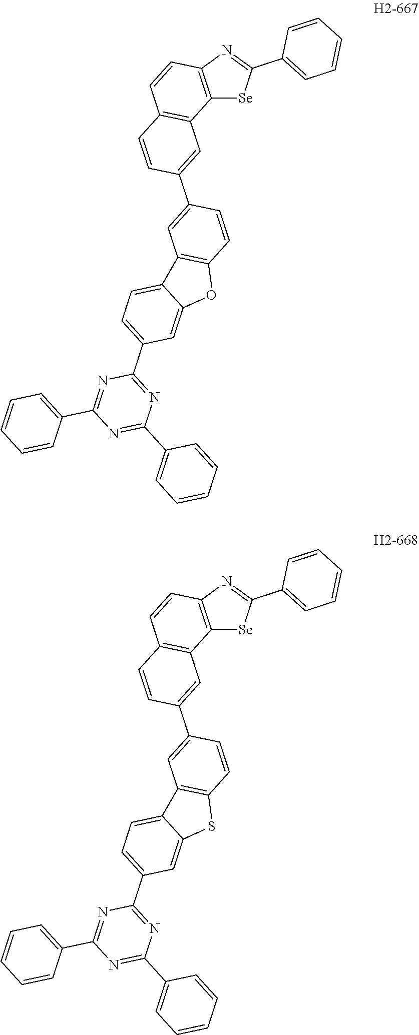 [SMILES: c1ccc(-c2nc(-c3ccccc3)nc(-c3ccc4c(c3)oc3ccc(-c5ccc6ccc7nc(-c8ccccc8)[se]c7c6c5)cc34)n2)cc1 ; c1ccc(-c2nc(-c3ccccc3)nc(-c3ccc4c(c3)sc3ccc(-c5ccc6ccc7nc(-c8ccccc8)[se]c7c6c5)cc34)n2)cc1]

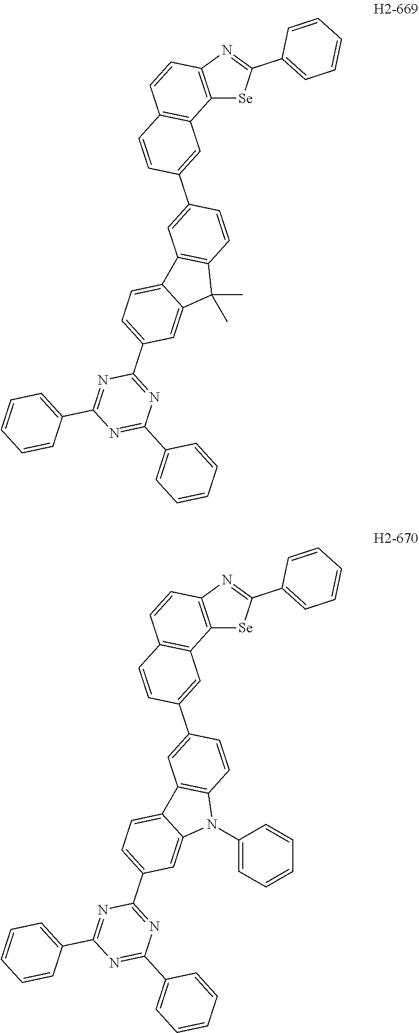 [SMILES: CC1(C)c2ccc(-c3ccc4ccc5nc(-c6ccccc6)[se]c5c4c3)cc2-c2ccc(-c3nc(-c4ccccc4)nc(-c4ccccc4)n3)cc21 ; c1ccc(-c2nc(-c3ccccc3)nc(-c3ccc4c5cc(-c6ccc7ccc8nc(-c9ccccc9)[se]c8c7c6)ccc5n(-c5ccccc5)c4c3)n2)cc1]

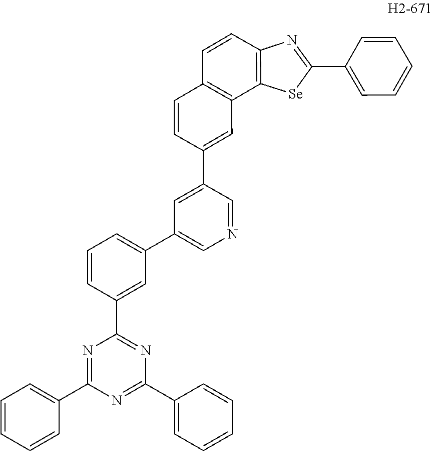 [SMILES: c1ccc(-c2nc(-c3ccccc3)nc(-c3cccc(-c4cncc(-c5ccc6ccc7nc(-c8ccccc8)[se]c7c6c5)c4)c3)n2)cc1]

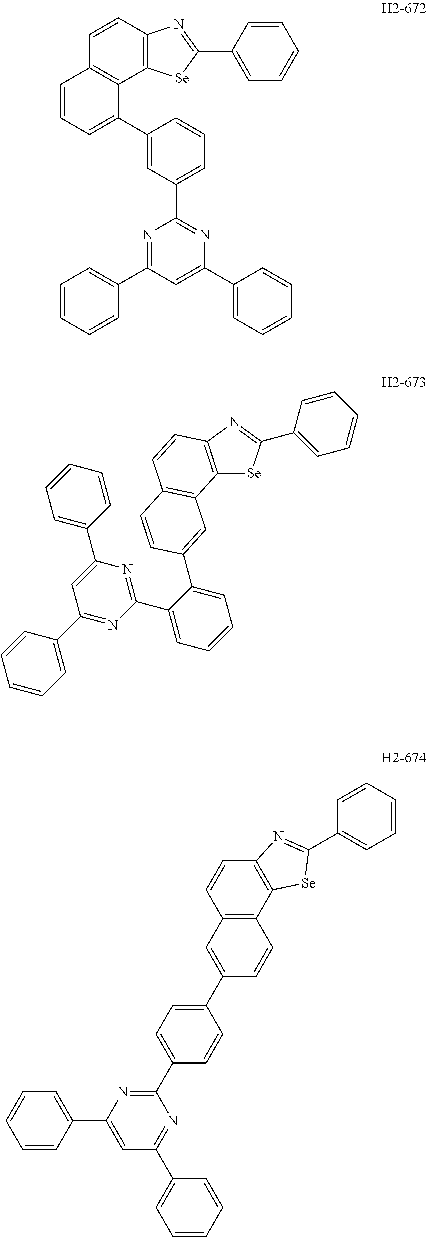 [SMILES: c1ccc(-c2cc(-c3ccccc3)nc(-c3cccc(-c4cccc5ccc6nc(-c7ccccc7)[se]c6c45)c3)n2)cc1 ; c1ccc(-c2cc(-c3ccccc3)nc(-c3ccccc3-c3ccc4ccc5nc(-c6ccccc6)[se]c5c4c3)n2)cc1 ; c1ccc(-c2cc(-c3ccccc3)nc(-c3ccc(-c4ccc5c(ccc6nc(-c7ccccc7)[se]c65)c4)cc3)n2)cc1]

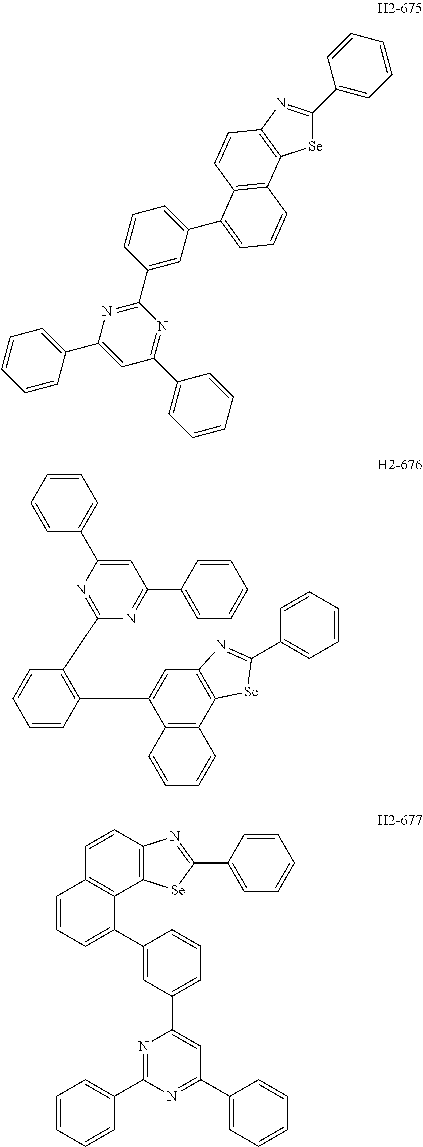 [SMILES: c1ccc(-c2cc(-c3ccccc3)nc(-c3cccc(-c4cccc5c4ccc4nc(-c6ccccc6)[se]c45)c3)n2)cc1 ; c1ccc(-c2cc(-c3ccccc3)nc(-c3ccccc3-c3cc4nc(-c5ccccc5)[se]c4c4ccccc34)n2)cc1 ; c1ccc(-c2cc(-c3cccc(-c4cccc5ccc6nc(-c7ccccc7)[se]c6c45)c3)nc(-c3ccccc3)n2)cc1]

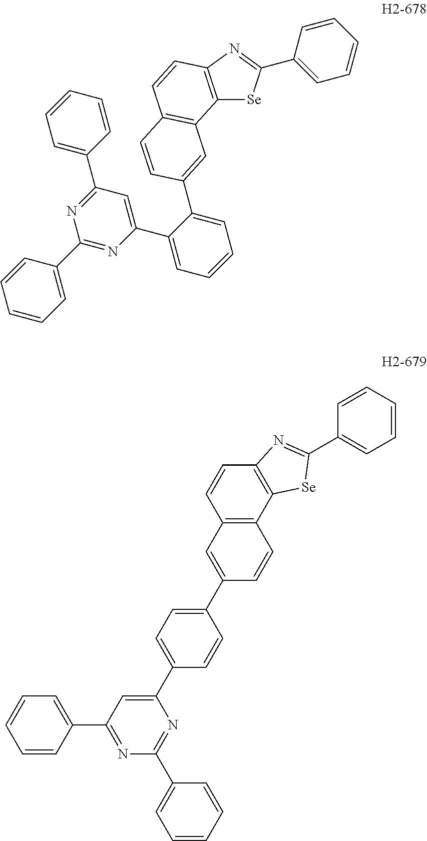 [SMILES: c1ccc(-c2cc(-c3ccccc3-c3ccc4ccc5nc(-c6ccccc6)[se]c5c4c3)nc(-c3ccccc3)n2)cc1 ; c1ccc(-c2cc(-c3ccc(-c4ccc5c(ccc6nc(-c7ccccc7)[se]c65)c4)cc3)nc(-c3ccccc3)n2)cc1]

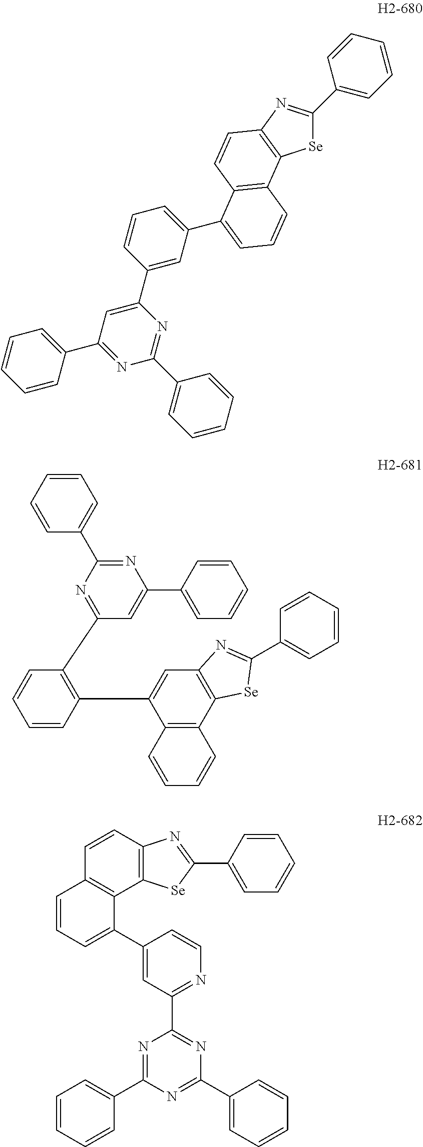 [SMILES: c1ccc(-c2cc(-c3cccc(-c4cccc5c4ccc4nc(-c6ccccc6)[se]c45)c3)nc(-c3ccccc3)n2)cc1 ; c1ccc(-c2cc(-c3ccccc3-c3cc4nc(-c5ccccc5)[se]c4c4ccccc34)nc(-c3ccccc3)n2)cc1 ; c1ccc(-c2nc(-c3ccccc3)nc(-c3cc(-c4cccc5ccc6nc(-c7ccccc7)[se]c6c45)ccn3)n2)cc1]

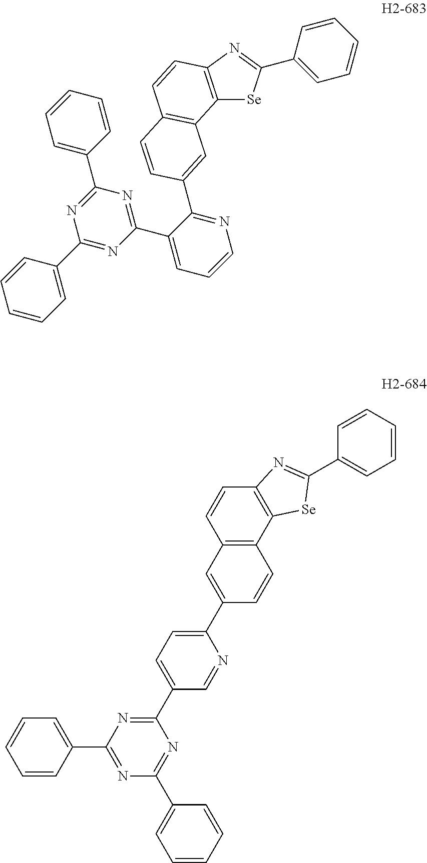 [SMILES: c1ccc(-c2nc(-c3ccccc3)nc(-c3cccnc3-c3ccc4ccc5nc(-c6ccccc6)[se]c5c4c3)n2)cc1 ; c1ccc(-c2nc(-c3ccccc3)nc(-c3ccc(-c4ccc5c(ccc6nc(-c7ccccc7)[se]c65)c4)nc3)n2)cc1]

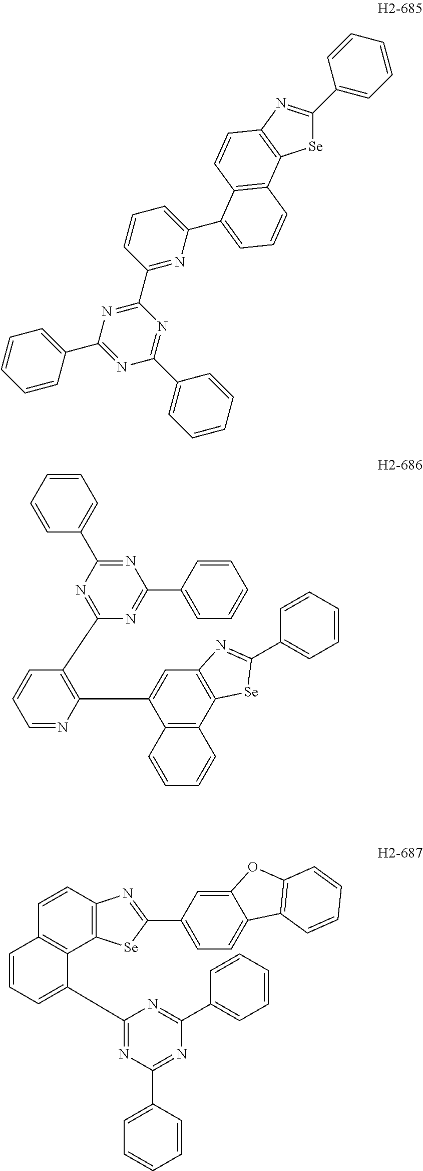 [SMILES: c1ccc(-c2nc(-c3ccccc3)nc(-c3cccc(-c4cccc5c4ccc4nc(-c6ccccc6)[se]c45)n3)n2)cc1 ; c1ccc(-c2nc(-c3ccccc3)nc(-c3cccnc3-c3cc4nc(-c5ccccc5)[se]c4c4ccccc34)n2)cc1 ; c1ccc(-c2nc(-c3ccccc3)nc(-c3cccc4ccc5nc(-c6ccc7c(c6)oc6ccccc67)[se]c5c34)n2)cc1]

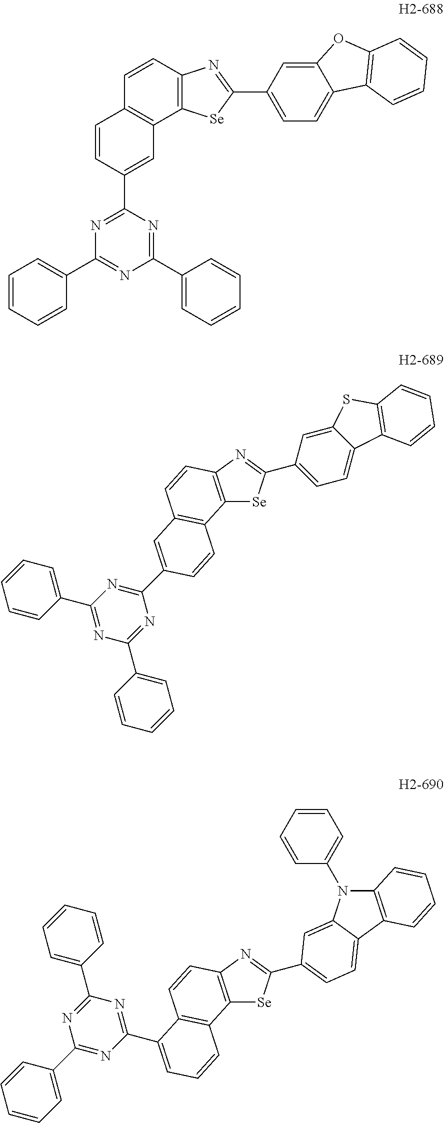 [SMILES: c1ccc(-c2nc(-c3ccccc3)nc(-c3ccc4ccc5nc(-c6ccc7c(c6)oc6ccccc67)[se]c5c4c3)n2)cc1 ; c1ccc(-c2nc(-c3ccccc3)nc(-c3ccc4c(ccc5nc(-c6ccc7c(c6)sc6ccccc67)[se]c54)c3)n2)cc1 ; c1ccc(-c2nc(-c3ccccc3)nc(-c3cccc4c3ccc3nc(-c5ccc6c7ccccc7n(-c7ccccc7)c6c5)[se]c34)n2)cc1]

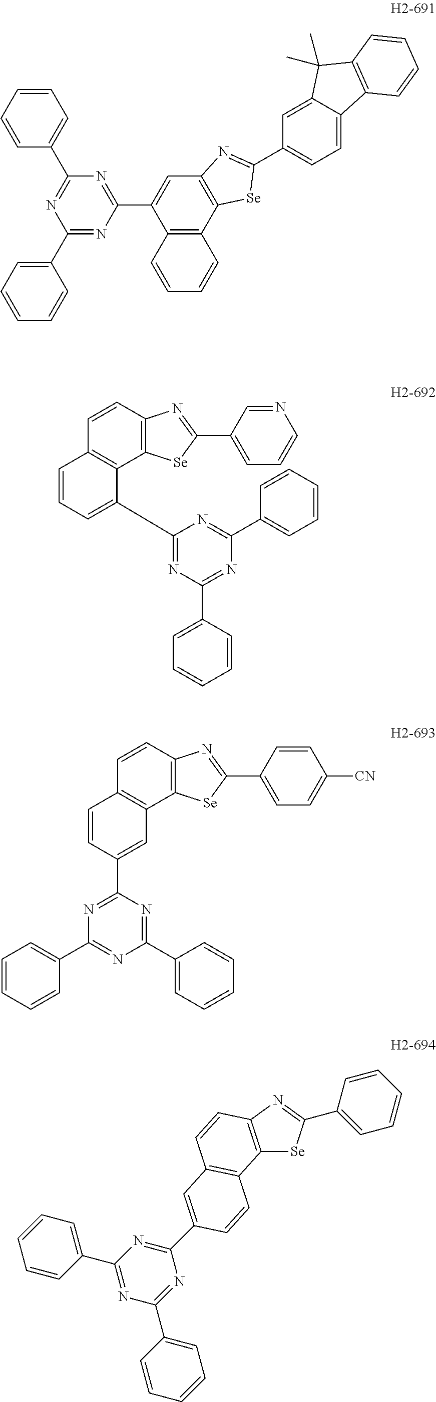 [SMILES: CC1(C)c2ccccc2-c2ccc(-c3nc4cc(-c5nc(-c6ccccc6)nc(-c6ccccc6)n5)c5ccccc5c4[se]3)cc21 ; c1ccc(-c2nc(-c3ccccc3)nc(-c3cccc4ccc5nc(-c6cccnc6)[se]c5c34)n2)cc1 ; N#Cc1ccc(-c2nc3ccc4ccc(-c5nc(-c6ccccc6)nc(-c6ccccc6)n5)cc4c3[se]2)cc1 ; c1ccc(-c2nc(-c3ccccc3)nc(-c3ccc4c(ccc5nc(-c6ccccc6)[se]c54)c3)n2)cc1]

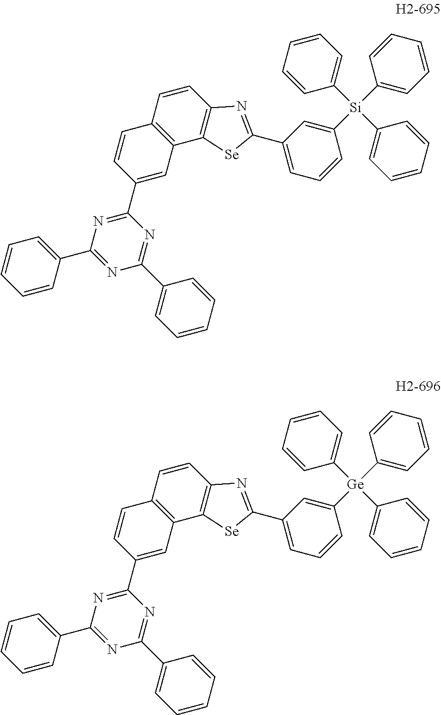

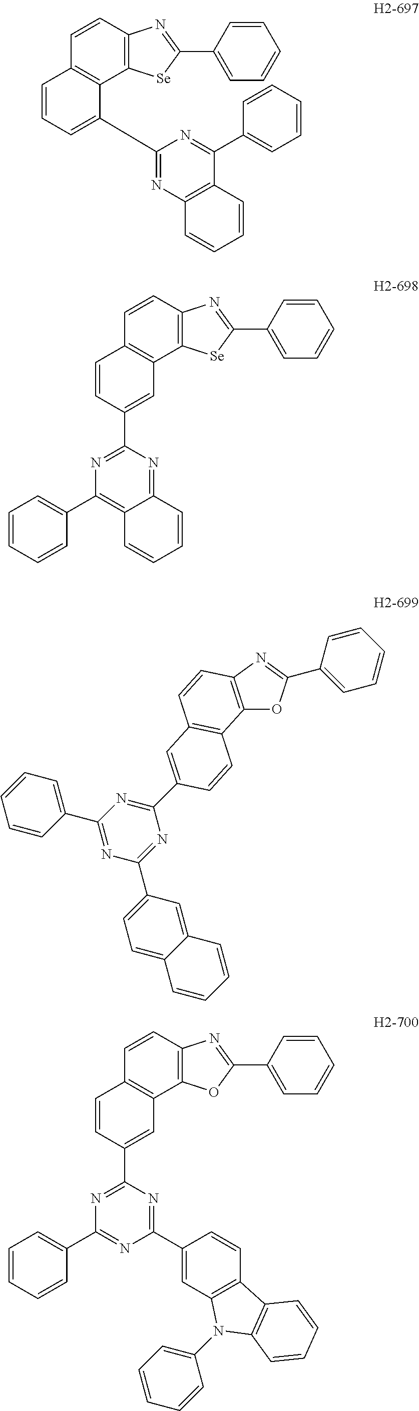

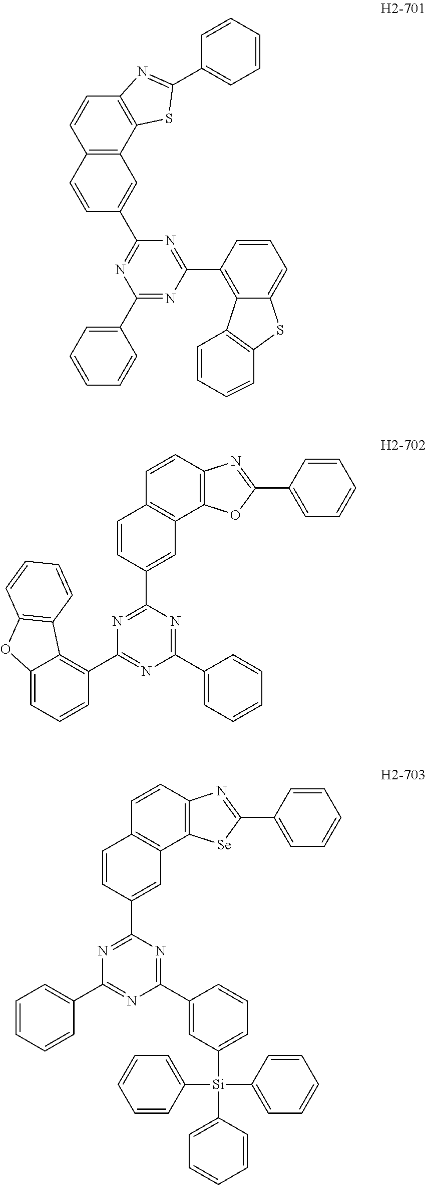 [SMILES: c1ccc(-c2nc(-c3ccc4ccc5nc(-c6ccccc6)sc5c4c3)nc(-c3cccc4sc5ccccc5c34)n2)cc1 ; c1ccc(-c2nc(-c3ccc4ccc5nc(-c6ccccc6)oc5c4c3)nc(-c3cccc4oc5ccccc5c34)n2)cc1 ; c1ccc(-c2nc(-c3cccc([Si](c4ccccc4)(c4ccccc4)c4ccccc4)c3)nc(-c3ccc4ccc5nc(-c6ccccc6)[se]c5c4c3)n2)cc1]

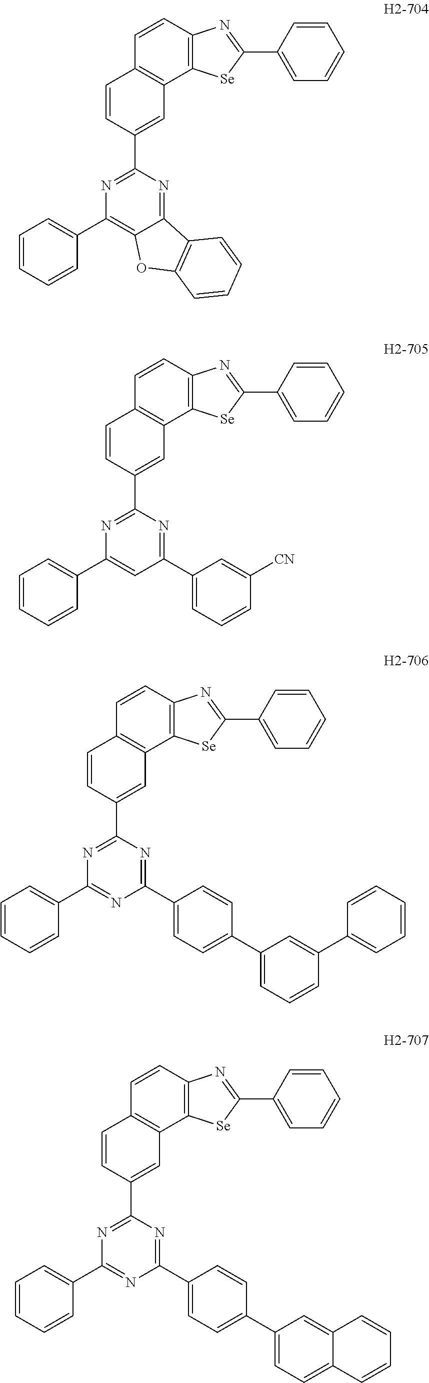 [SMILES: c1ccc(-c2nc3ccc4ccc(-c5nc(-c6ccccc6)c6oc7ccccc7c6n5)cc4c3[se]2)cc1 ; N#Cc1cccc(-c2cc(-c3ccccc3)nc(-c3ccc4ccc5nc(-c6ccccc6)[se]c5c4c3)n2)c1 ; c1ccc(-c2cccc(-c3ccc(-c4nc(-c5ccccc5)nc(-c5ccc6ccc7nc(-c8ccccc8)[se]c7c6c5)n4)cc3)c2)cc1 ; c1ccc(-c2nc(-c3ccc(-c4ccc5ccccc5c4)cc3)nc(-c3ccc4ccc5nc(-c6ccccc6)[se]c5c4c3)n2)cc1]

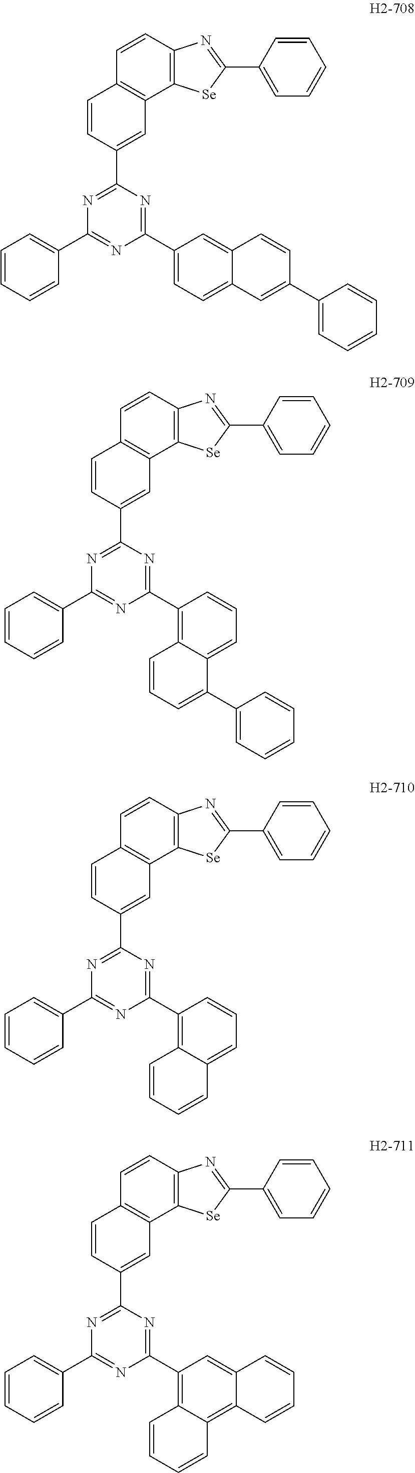

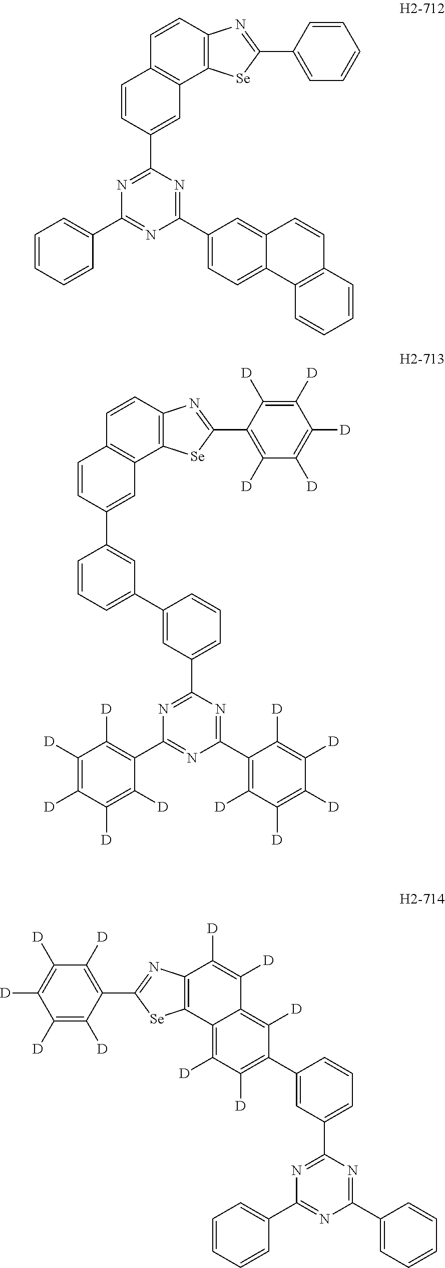

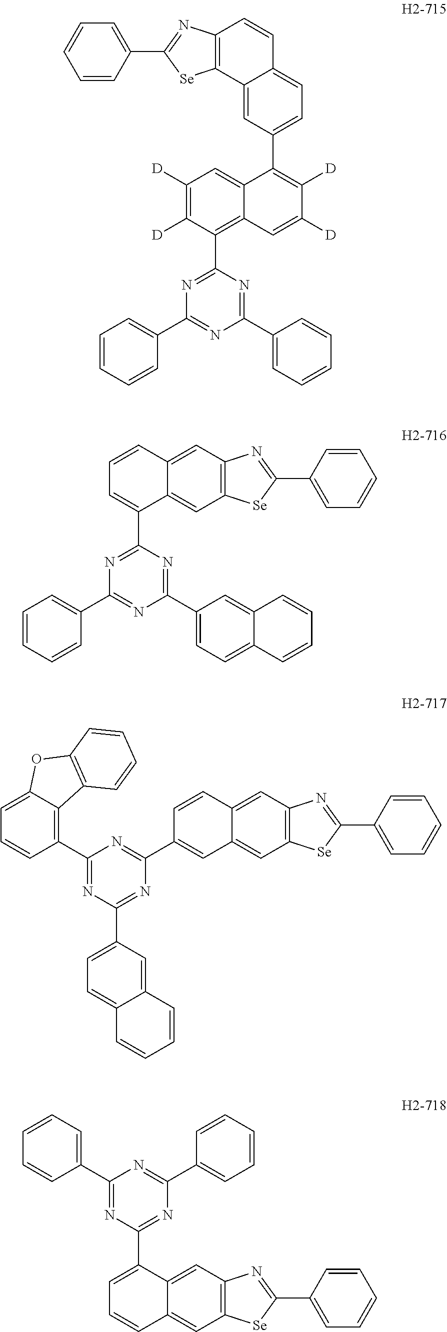 [SMILES: [2H]c1cc2c(-c3nc(-c4ccccc4)nc(-c4ccccc4)n3)c([2H])c([2H])cc2c(-c2ccc3ccc4nc(-c5ccccc5)[se]c4c3c2)c1[2H] ; c1ccc(-c2nc(-c3ccc4ccccc4c3)nc(-c3cccc4cc5nc(-c6ccccc6)[se]c5cc34)n2)cc1 ; c1ccc(-c2nc3cc4ccc(-c5nc(-c6ccc7ccccc7c6)nc(-c6cccc7oc8ccccc8c67)n5)cc4cc3[se]2)cc1 ; c1ccc(-c2nc(-c3ccccc3)nc(-c3cccc4cc5[se]c(-c6ccccc6)nc5cc34)n2)cc1]

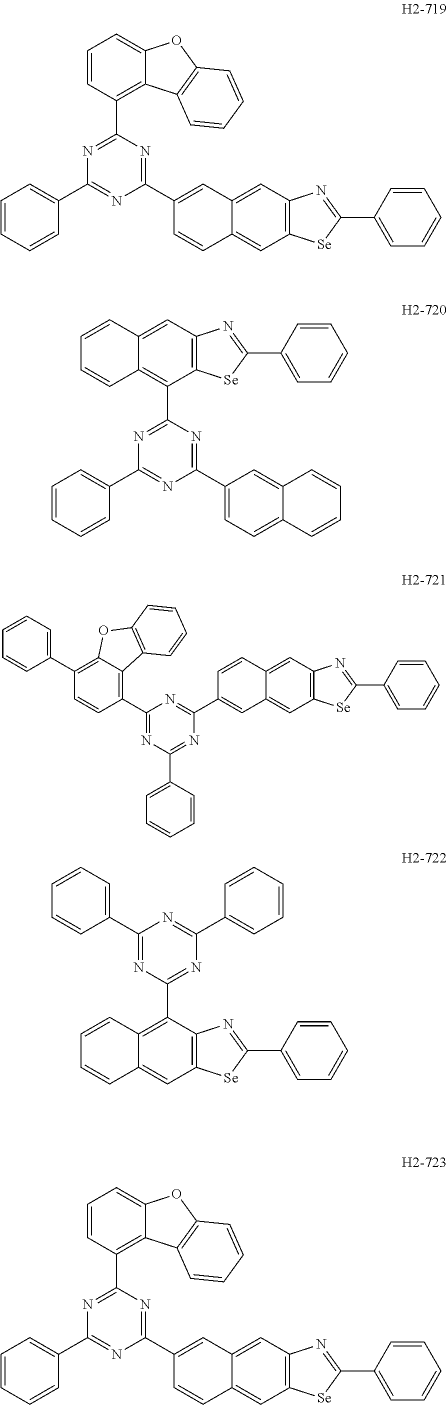 [SMILES: c1ccc(-c2nc(-c3ccc4cc5[se]c(-c6ccccc6)nc5cc4c3)nc(-c3cccc4oc5ccccc5c34)n2)cc1 ; c1ccc(-c2nc(-c3ccc4ccccc4c3)nc(-c3c4ccccc4cc4nc(-c5ccccc5)[se]c34)n2)cc1 ; c1ccc(-c2nc(-c3ccc4cc5nc(-c6ccccc6)[se]c5cc4c3)nc(-c3ccc(-c4ccccc4)c4oc5ccccc5c34)n2)cc1 ; c1ccc(-c2nc(-c3ccccc3)nc(-c3c4ccccc4cc4[se]c(-c5ccccc5)nc34)n2)cc1 ; c1ccc(-c2nc(-c3ccc4cc5[se]c(-c6ccccc6)nc5cc4c3)nc(-c3cccc4oc5ccccc5c34)n2)cc1]

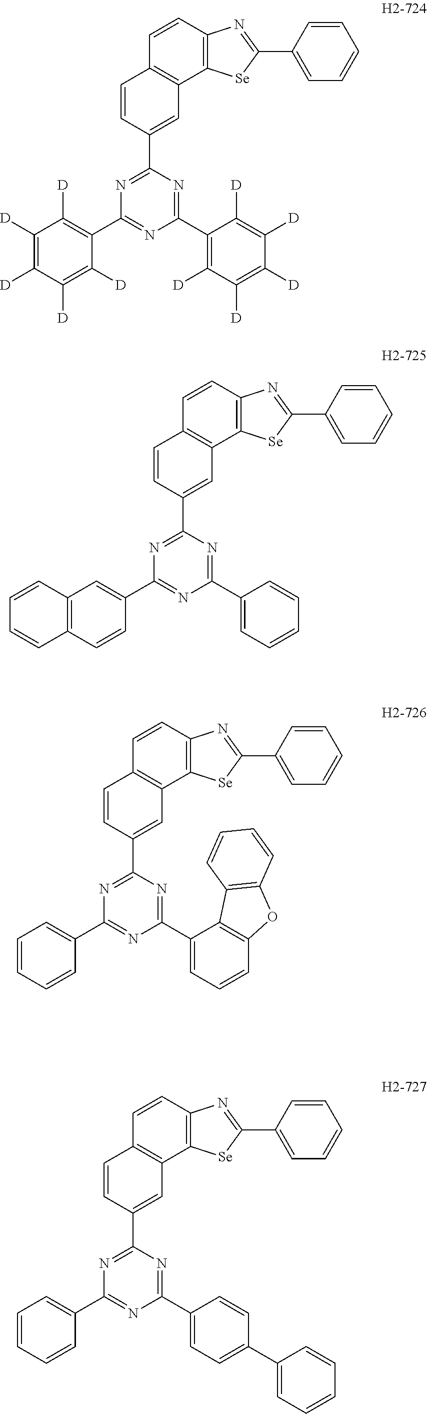 [SMILES: [2H]c1c([2H])c([2H])c(-c2nc(-c3ccc4ccc5nc(-c6ccccc6)[se]c5c4c3)nc(-c3c([2H])c([2H])c([2H])c([2H])c3[2H])n2)c([2H])c1[2H] ; c1ccc(-c2nc(-c3ccc4ccccc4c3)nc(-c3ccc4ccc5nc(-c6ccccc6)[se]c5c4c3)n2)cc1 ; c1ccc(-c2nc(-c3ccc4ccc5nc(-c6ccccc6)[se]c5c4c3)nc(-c3cccc4oc5ccccc5c34)n2)cc1 ; c1ccc(-c2ccc(-c3nc(-c4ccccc4)nc(-c4ccc5ccc6nc(-c7ccccc7)[se]c6c5c4)n3)cc2)cc1]

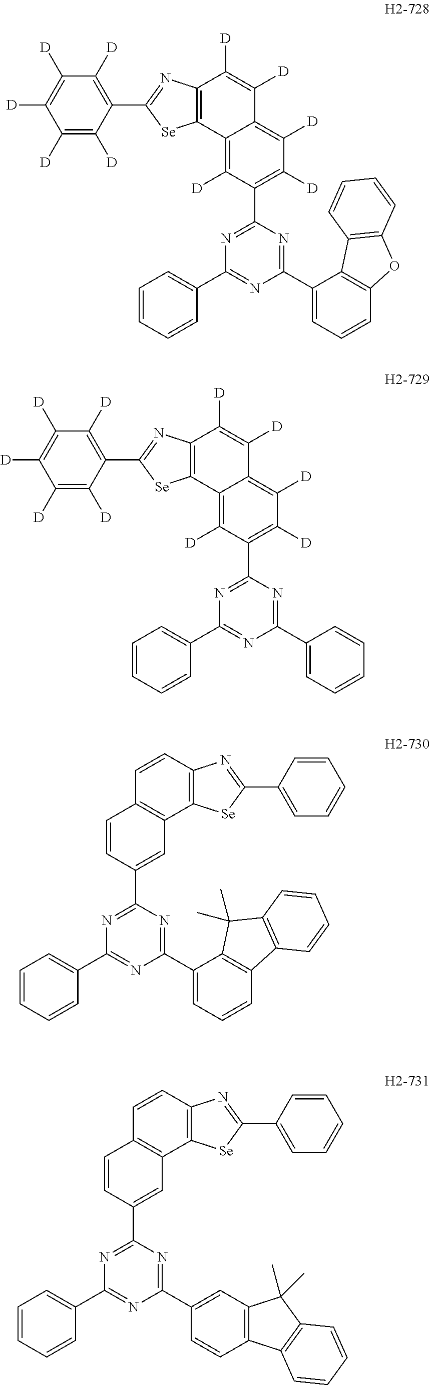 [SMILES: [2H]c1c([2H])c([2H])c(-c2nc3c([2H])c([2H])c4c([2H])c([2H])c(-c5nc(-c6ccccc6)nc(-c6cccc7oc8ccccc8c67)n5)c([2H])c4c3[se]2)c([2H])c1[2H] ; [2H]c1c([2H])c([2H])c(-c2nc3c([2H])c([2H])c4c([2H])c([2H])c(-c5nc(-c6ccccc6)nc(-c6ccccc6)n5)c([2H])c4c3[se]2)c([2H])c1[2H] ; CC1(C)c2ccccc2-c2cccc(-c3nc(-c4ccccc4)nc(-c4ccc5ccc6nc(-c7ccccc7)[se]c6c5c4)n3)c21 ; CC1(C)c2ccccc2-c2ccc(-c3nc(-c4ccccc4)nc(-c4ccc5ccc6nc(-c7ccccc7)[se]c6c5c4)n3)cc21]

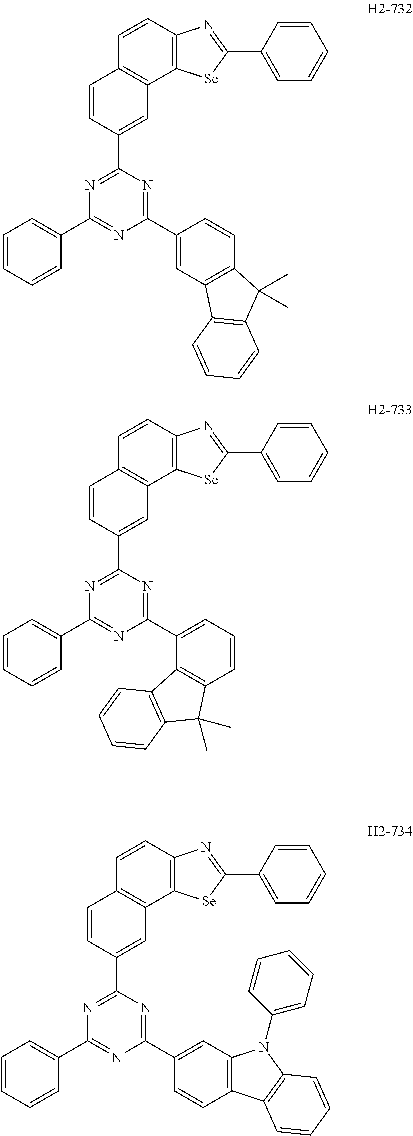 [SMILES: CC1(C)c2ccccc2-c2cc(-c3nc(-c4ccccc4)nc(-c4ccc5ccc6nc(-c7ccccc7)[se]c6c5c4)n3)ccc21 ; CC1(C)c2ccccc2-c2c(-c3nc(-c4ccccc4)nc(-c4ccc5ccc6nc(-c7ccccc7)[se]c6c5c4)n3)cccc21 ; c1ccc(-c2nc(-c3ccc4ccc5nc(-c6ccccc6)[se]c5c4c3)nc(-c3ccc4c5ccccc5n(-c5ccccc5)c4c3)n2)cc1]

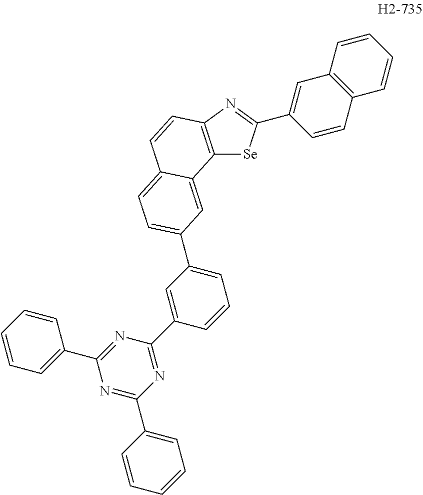 [SMILES: c1ccc(-c2nc(-c3ccccc3)nc(-c3cccc(-c4ccc5ccc6nc(-c7ccc8ccccc8c7)[se]c6c5c4)c3)n2)cc1]

The compound represented by any one of formulas 2′-1 to 2′-4 may be at least one selected from the group consisting of the following compounds, but is not limited thereto.

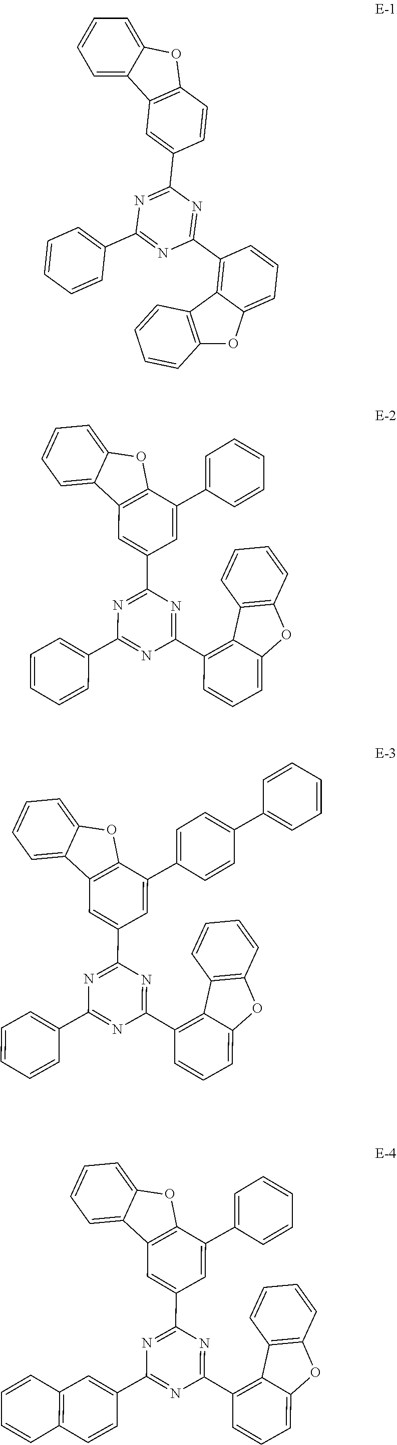 [SMILES: c1ccc(-c2nc(-c3ccc4oc5ccccc5c4c3)nc(-c3cccc4oc5ccccc5c34)n2)cc1 ; c1ccc(-c2nc(-c3cc(-c4ccccc4)c4oc5ccccc5c4c3)nc(-c3cccc4oc5ccccc5c34)n2)cc1 ; c1ccc(-c2ccc(-c3cc(-c4nc(-c5ccccc5)nc(-c5cccc6oc7ccccc7c56)n4)cc4c3oc3ccccc34)cc2)cc1 ; c1ccc(-c2cc(-c3nc(-c4ccc5ccccc5c4)nc(-c4cccc5oc6ccccc6c45)n3)cc3c2oc2ccccc23)cc1]

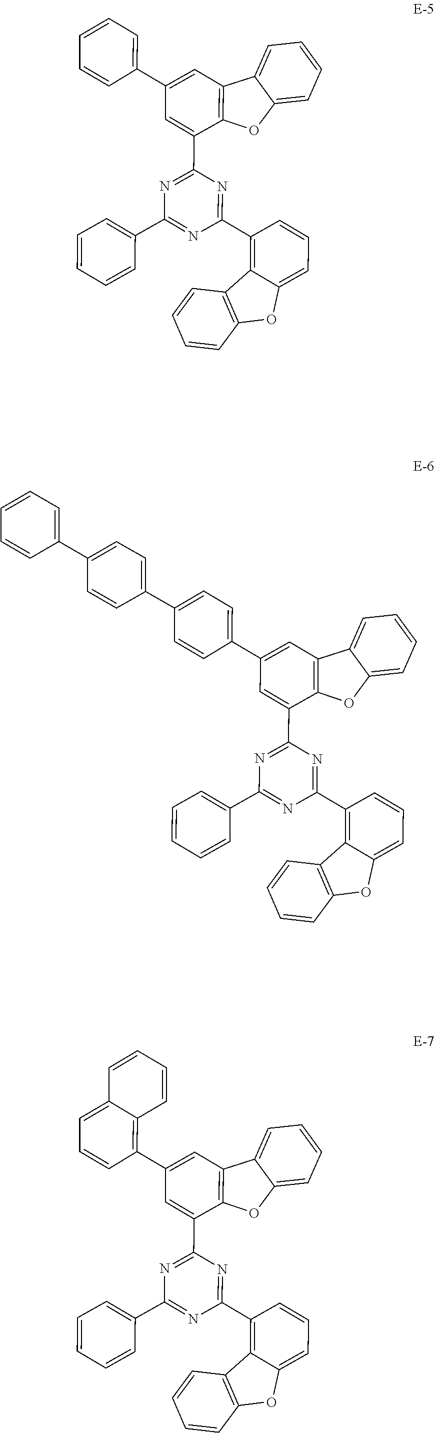 [SMILES: c1ccc(-c2cc(-c3nc(-c4ccccc4)nc(-c4cccc5oc6ccccc6c45)n3)c3oc4ccccc4c3c2)cc1 ; c1ccc(-c2ccc(-c3ccc(-c4cc(-c5nc(-c6ccccc6)nc(-c6cccc7oc8ccccc8c67)n5)c5oc6ccccc6c5c4)cc3)cc2)cc1 ; c1ccc(-c2nc(-c3cc(-c4cccc5ccccc45)cc4c3oc3ccccc34)nc(-c3cccc4oc5ccccc5c34)n2)cc1]

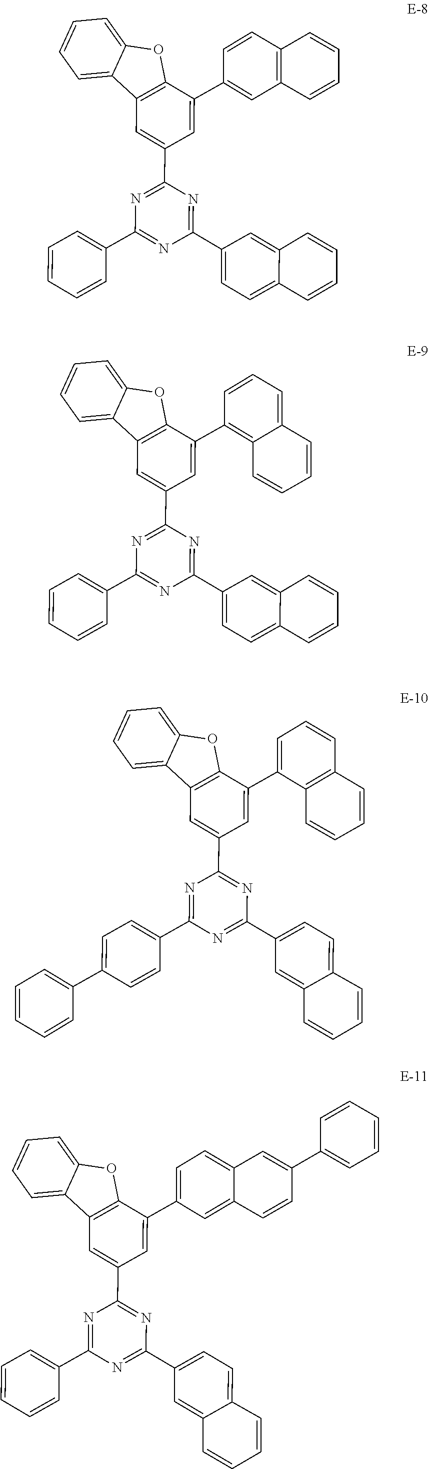 [SMILES: c1ccc(-c2nc(-c3ccc4ccccc4c3)nc(-c3cc(-c4ccc5ccccc5c4)c4oc5ccccc5c4c3)n2)cc1 ; c1ccc(-c2nc(-c3ccc4ccccc4c3)nc(-c3cc(-c4cccc5ccccc45)c4oc5ccccc5c4c3)n2)cc1 ; c1ccc(-c2ccc(-c3nc(-c4ccc5ccccc5c4)nc(-c4cc(-c5cccc6ccccc56)c5oc6ccccc6c5c4)n3)cc2)cc1 ; c1ccc(-c2ccc3cc(-c4cc(-c5nc(-c6ccccc6)nc(-c6ccc7ccccc7c6)n5)cc5c4oc4ccccc45)ccc3c2)cc1]

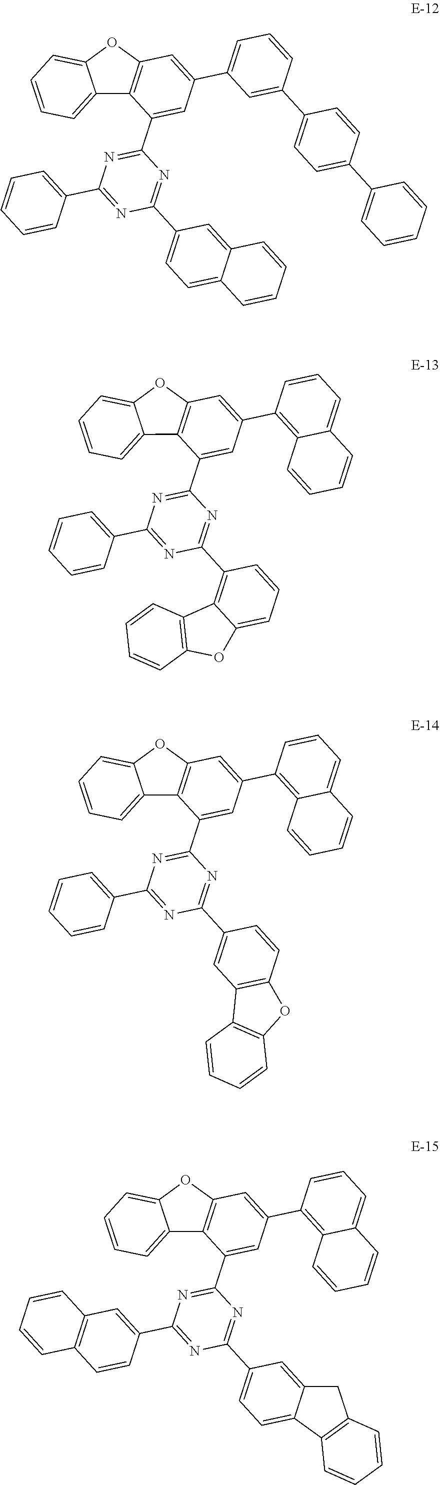 [SMILES: c1ccc(-c2ccc(-c3cccc(-c4cc(-c5nc(-c6ccccc6)nc(-c6ccc7ccccc7c6)n5)c5c(c4)oc4ccccc45)c3)cc2)cc1 ; c1ccc(-c2nc(-c3cccc4oc5ccccc5c34)nc(-c3cc(-c4cccc5ccccc45)cc4oc5ccccc5c34)n2)cc1 ; c1ccc(-c2nc(-c3ccc4oc5ccccc5c4c3)nc(-c3cc(-c4cccc5ccccc45)cc4oc5ccccc5c34)n2)cc1 ; c1ccc2c(c1)Cc1cc(-c3nc(-c4ccc5ccccc5c4)nc(-c4cc(-c5cccc6ccccc56)cc5oc6ccccc6c45)n3)ccc1-2]

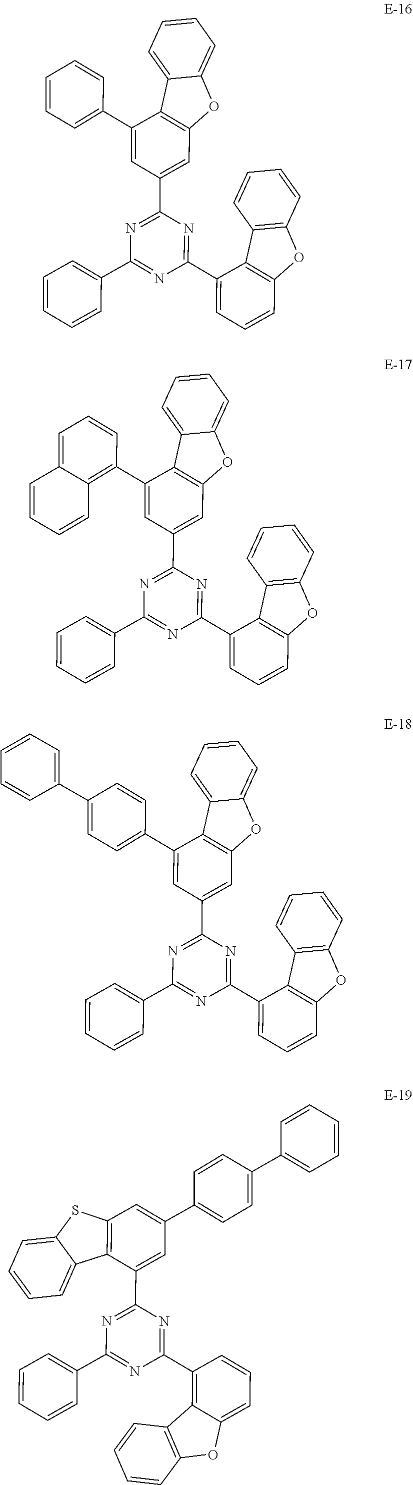 [SMILES: c1ccc(-c2nc(-c3cc(-c4ccccc4)c4c(c3)oc3ccccc34)nc(-c3cccc4oc5ccccc5c34)n2)cc1 ; c1ccc(-c2nc(-c3cc(-c4cccc5ccccc45)c4c(c3)oc3ccccc34)nc(-c3cccc4oc5ccccc5c34)n2)cc1 ; c1ccc(-c2ccc(-c3cc(-c4nc(-c5ccccc5)nc(-c5cccc6oc7ccccc7c56)n4)cc4oc5ccccc5c34)cc2)cc1 ; c1ccc(-c2ccc(-c3cc(-c4nc(-c5ccccc5)nc(-c5cccc6oc7ccccc7c56)n4)c4c(c3)sc3ccccc34)cc2)cc1]

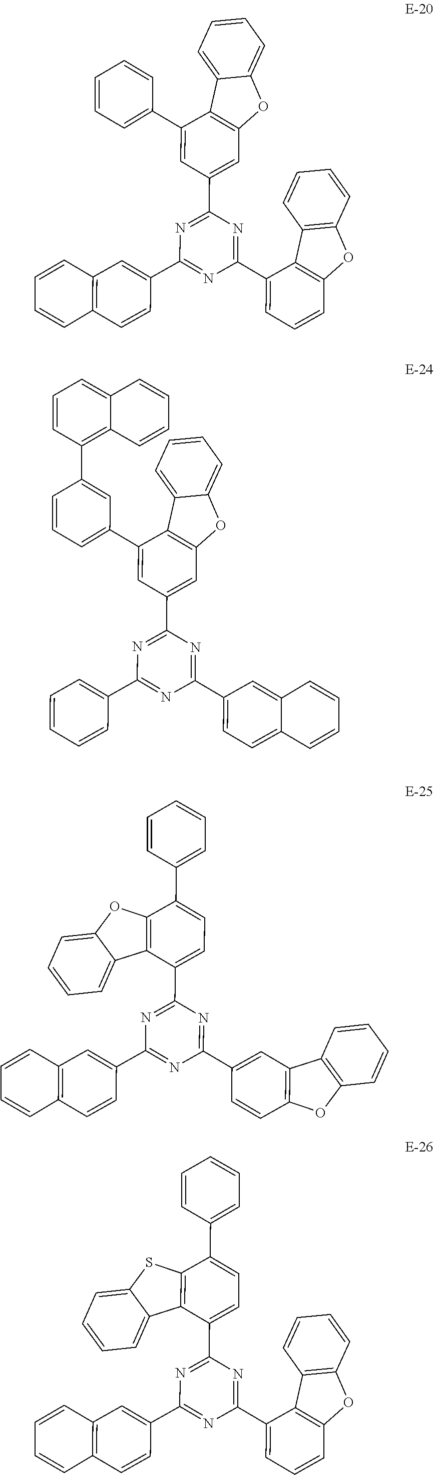 [SMILES: c1ccc(-c2cc(-c3nc(-c4ccc5ccccc5c4)nc(-c4cccc5oc6ccccc6c45)n3)cc3oc4ccccc4c23)cc1 ; c1ccc(-c2nc(-c3ccc4ccccc4c3)nc(-c3cc(-c4cccc(-c5cccc6ccccc56)c4)c4c(c3)oc3ccccc34)n2)cc1 ; c1ccc(-c2ccc(-c3nc(-c4ccc5ccccc5c4)nc(-c4ccc5oc6ccccc6c5c4)n3)c3c2oc2ccccc23)cc1 ; c1ccc(-c2ccc(-c3nc(-c4ccc5ccccc5c4)nc(-c4cccc5oc6ccccc6c45)n3)c3c2sc2ccccc23)cc1]

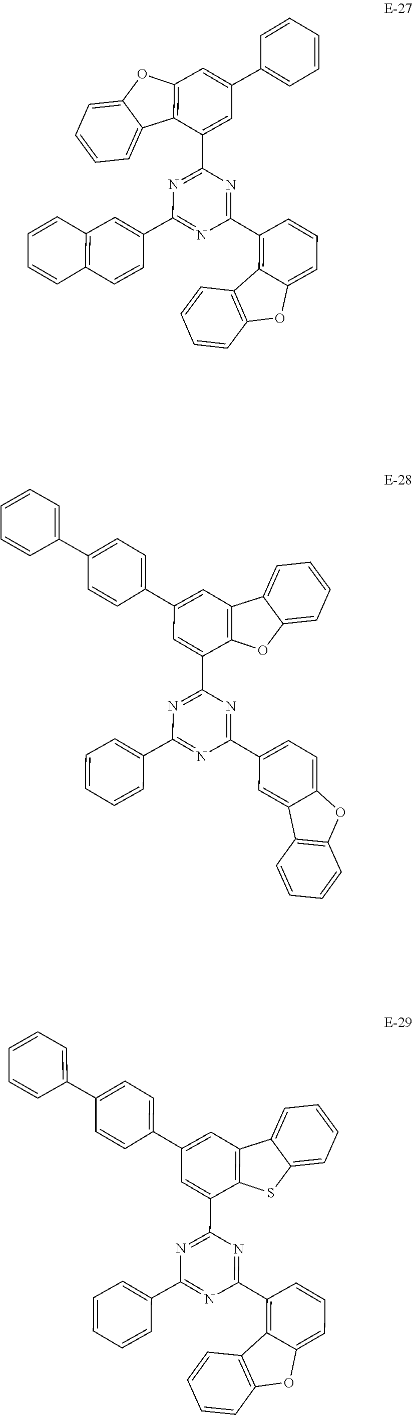 [SMILES: c1ccc(-c2cc(-c3nc(-c4ccc5ccccc5c4)nc(-c4cccc5oc6ccccc6c45)n3)c3c(c2)oc2ccccc23)cc1 ; c1ccc(-c2ccc(-c3cc(-c4nc(-c5ccccc5)nc(-c5ccc6oc7ccccc7c6c5)n4)c4oc5ccccc5c4c3)cc2)cc1 ; c1ccc(-c2ccc(-c3cc(-c4nc(-c5ccccc5)nc(-c5cccc6oc7ccccc7c56)n4)c4sc5ccccc5c4c3)cc2)cc1]

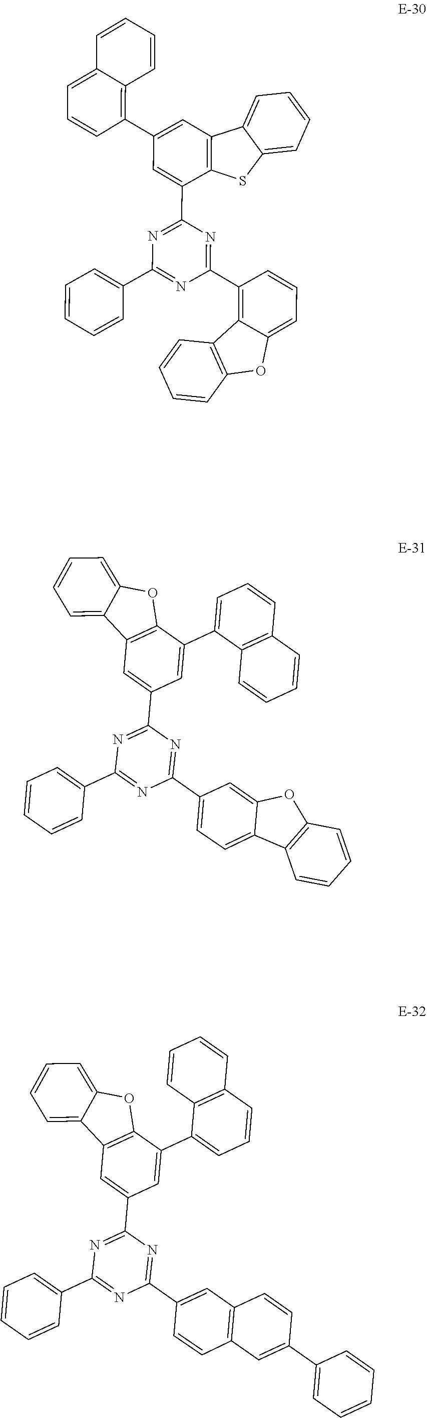 [SMILES: c1ccc(-c2nc(-c3cc(-c4cccc5ccccc45)cc4c3sc3ccccc34)nc(-c3cccc4oc5ccccc5c34)n2)cc1 ; c1ccc(-c2nc(-c3ccc4c(c3)oc3ccccc34)nc(-c3cc(-c4cccc5ccccc45)c4oc5ccccc5c4c3)n2)cc1 ; c1ccc(-c2ccc3cc(-c4nc(-c5ccccc5)nc(-c5cc(-c6cccc7ccccc67)c6oc7ccccc7c6c5)n4)ccc3c2)cc1]

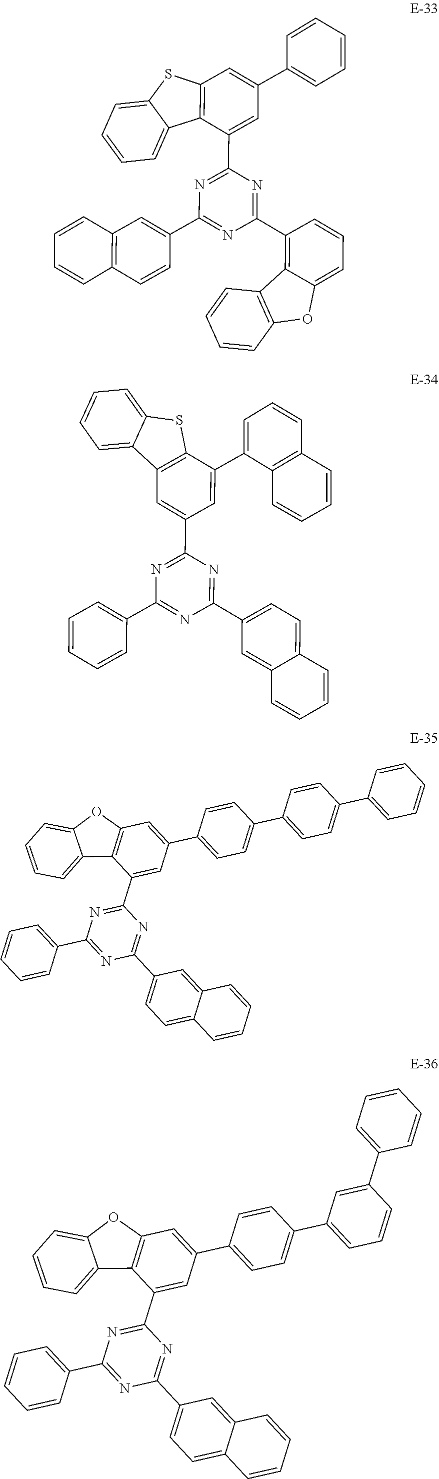 [SMILES: c1ccc(-c2cc(-c3nc(-c4ccc5ccccc5c4)nc(-c4cccc5oc6ccccc6c45)n3)c3c(c2)sc2ccccc23)cc1 ; c1ccc(-c2nc(-c3ccc4ccccc4c3)nc(-c3cc(-c4cccc5ccccc45)c4sc5ccccc5c4c3)n2)cc1 ; c1ccc(-c2ccc(-c3ccc(-c4cc(-c5nc(-c6ccccc6)nc(-c6ccc7ccccc7c6)n5)c5c(c4)oc4ccccc45)cc3)cc2)cc1 ; c1ccc(-c2cccc(-c3ccc(-c4cc(-c5nc(-c6ccccc6)nc(-c6ccc7ccccc7c6)n5)c5c(c4)oc4ccccc45)cc3)c2)cc1]

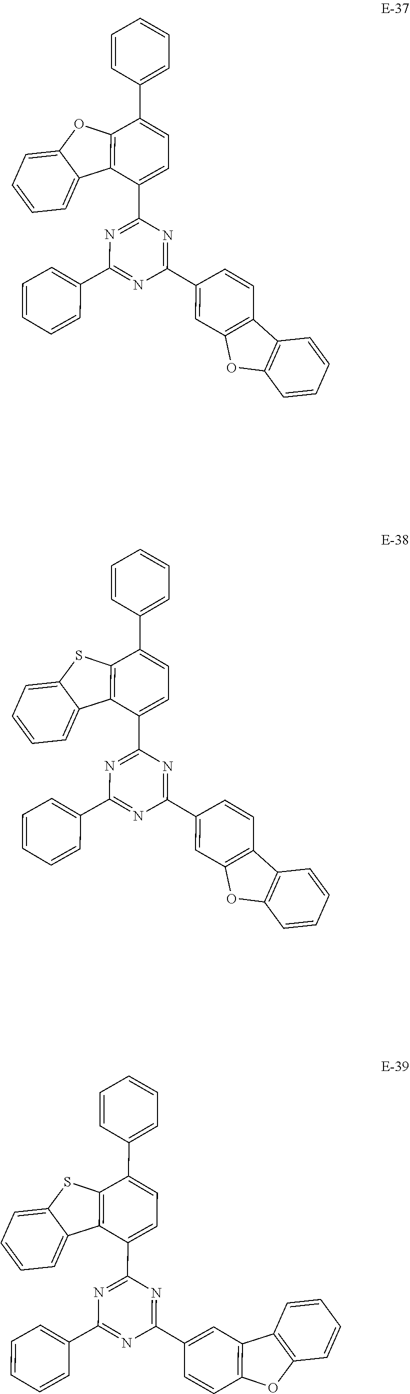 [SMILES: c1ccc(-c2nc(-c3ccc4c(c3)oc3ccccc34)nc(-c3ccc(-c4ccccc4)c4oc5ccccc5c34)n2)cc1 ; c1ccc(-c2nc(-c3ccc4c(c3)oc3ccccc34)nc(-c3ccc(-c4ccccc4)c4sc5ccccc5c34)n2)cc1 ; c1ccc(-c2nc(-c3ccc4oc5ccccc5c4c3)nc(-c3ccc(-c4ccccc4)c4sc5ccccc5c34)n2)cc1]

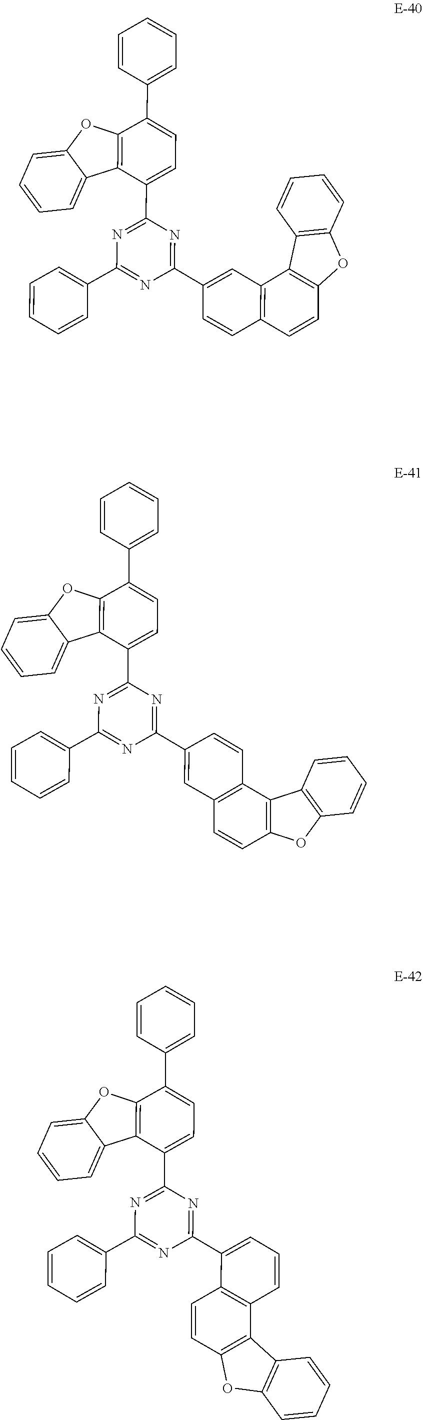 [SMILES: c1ccc(-c2nc(-c3ccc4ccc5oc6ccccc6c5c4c3)nc(-c3ccc(-c4ccccc4)c4oc5ccccc5c34)n2)cc1 ; c1ccc(-c2nc(-c3ccc4c(ccc5oc6ccccc6c54)c3)nc(-c3ccc(-c4ccccc4)c4oc5ccccc5c34)n2)cc1 ; c1ccc(-c2nc(-c3cccc4c3ccc3oc5ccccc5c34)nc(-c3ccc(-c4ccccc4)c4oc5ccccc5c34)n2)cc1]

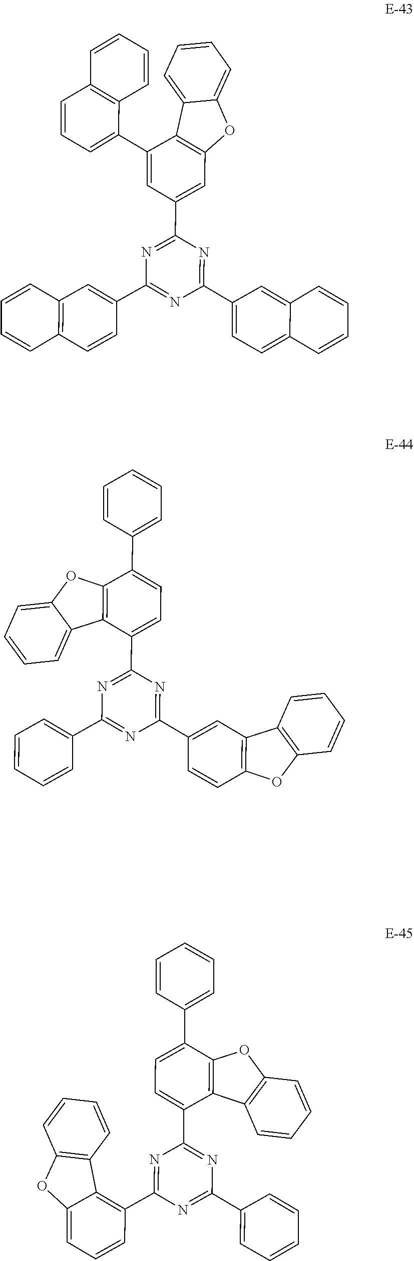 [SMILES: c1ccc2cc(-c3nc(-c4ccc5ccccc5c4)nc(-c4cc(-c5cccc6ccccc56)c5c(c4)oc4ccccc45)n3)ccc2c1 ; c1ccc(-c2nc(-c3ccc4oc5ccccc5c4c3)nc(-c3ccc(-c4ccccc4)c4oc5ccccc5c34)n2)cc1 ; c1ccc(-c2nc(-c3cccc4oc5ccccc5c34)nc(-c3ccc(-c4ccccc4)c4oc5ccccc5c34)n2)cc1]

The compounds represented by formula 1 or 1′ according to the present disclosure may be produced by a synthetic method known to one skilled in the art, for example, may be produced by referring to the following reaction schemes 1 to 6, but are not limited thereto.

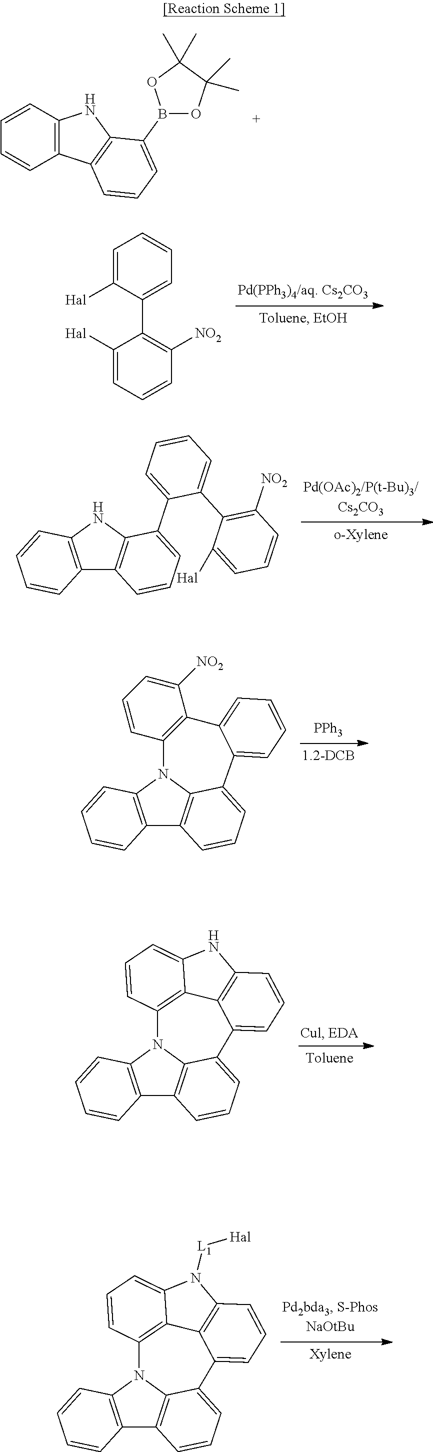

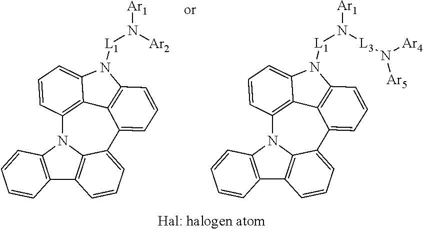

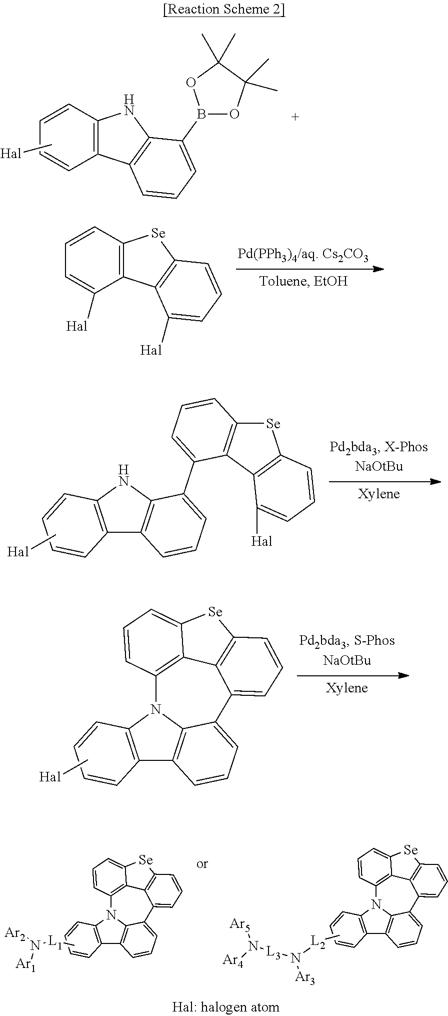

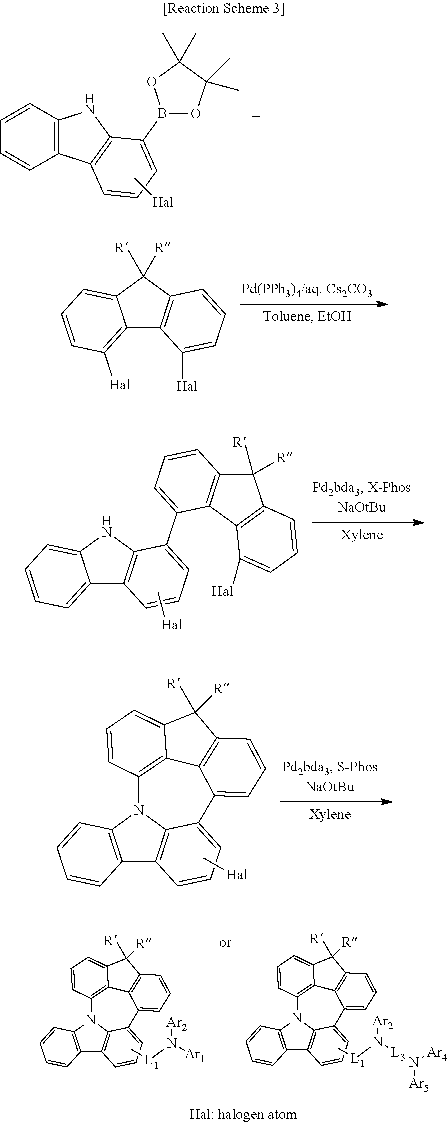

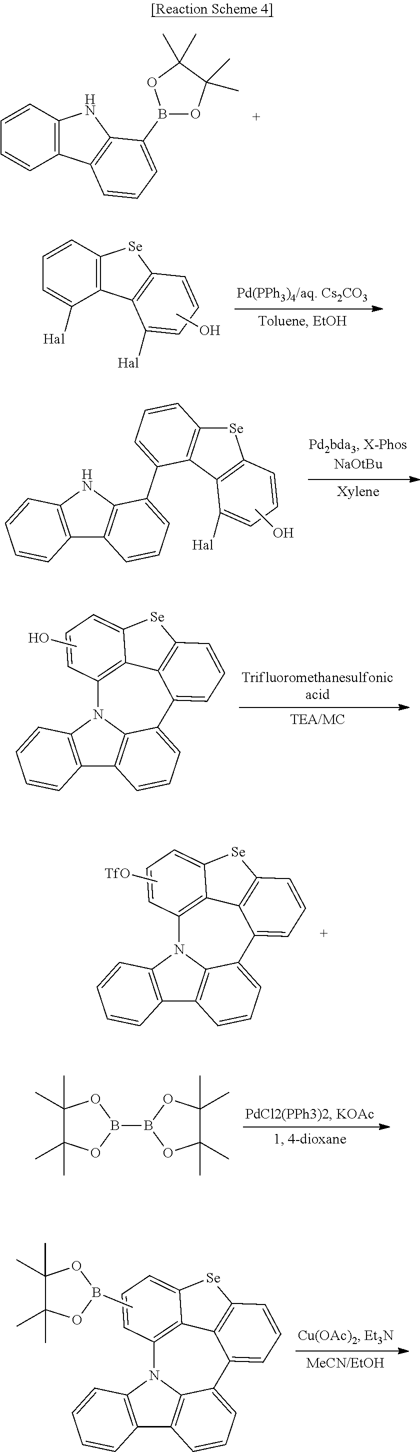

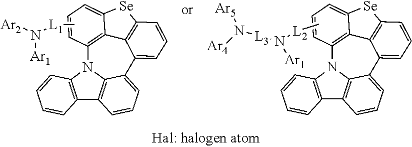

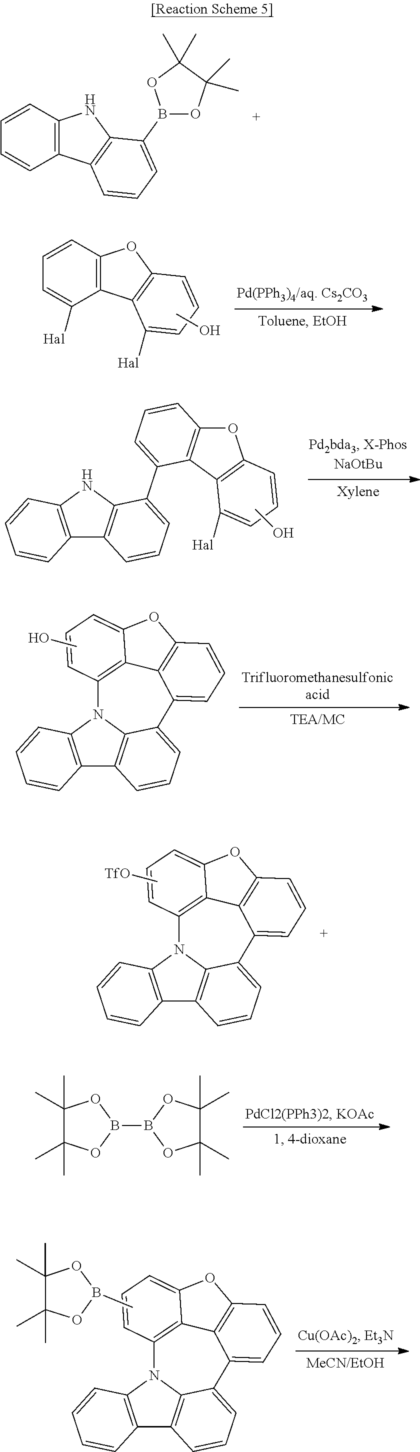

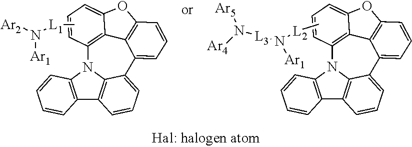

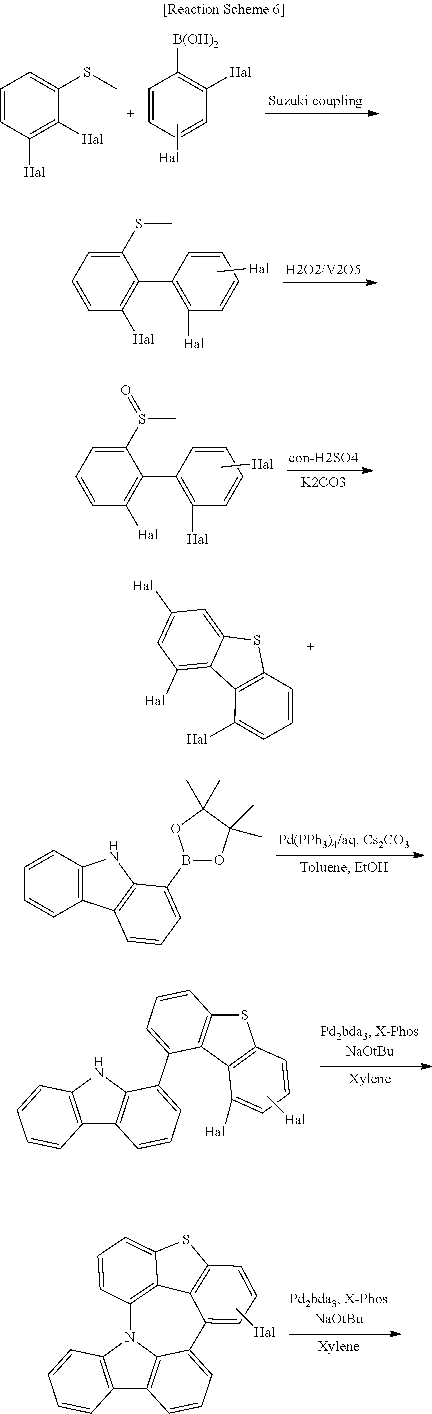

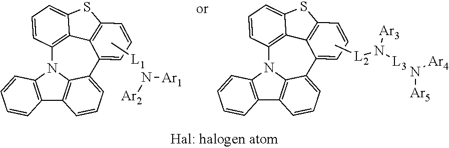

In reaction schemes 1 to 6, R′, R″, L₁ to L₃, and Ar₁ to Ar₅ are as defined in formula 1.

Although illustrative synthesis examples of the compounds represented by formula 1 or 1′ of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(Il)-catalyzed oxidative cyclization reaction, a Grignard reaction, a Heck reaction, a Cyclic Dehydration reaction, an SN1 substitution reaction, an SN2 substitution reaction, and a Phosphine-mediated reductive cyclization reaction, etc., and the reactions above proceed even if substituents which are defined in formula 1 or 1′ above, but are not specified in the specific synthesis examples, are bonded.

The host compound represented by formula 2 according to the present disclosure may be produced by a synthetic method known to one skilled in the art, for example, by referring to the method disclosed in Korean Patent Application Laying-Open No. 10-2020-0092879, etc., but is not limited thereto.

The host compounds represented by formulas 2′-1 to 2′-4 according to the present disclosure may be produced, for example, by referring to the method disclosed in Korean Patent Application Laying-Open No. 10-2014-0099082, International Publication No. 2015/169412 A1, Korean Patent Application Laying-Open No. 10-2018-0112962, etc., but are not limited thereto.

Hereinafter, an organic electroluminescent device to which the plurality of host materials and/or the organic electroluminescent compounds are applied will be described.

An organic electroluminescent device according to the one embodiment comprises an anode; a cathode; and at least one light-emitting layer between the anode and cathode, in which at least one light-emitting layer may comprise the plurality of host materials comprising at least one first host compound represented by formula 1 and at least one second host compound represented by formula 2. In this case, the weight ratio of the first host compound and the second host compound may be comprised in the light-emitting layer in the range of about 1:99 to about 99:1, preferably about 10:90 to about 90:10, more preferably about 30:70 to about 70:30, more preferably about 40:60 to about 60:40, and further more preferably about 50:50.

The plurality of host materials of the present disclosure may be comprised in the same organic layer, for example, in the same light-emitting layer, or may be comprised in different light-emitting layers.

According to another embodiment of the present disclosure, an organic electroluminescent compound represented by formula 1′ or any one of formulas 2′-1 to 2′-4 of the present disclosure may be comprised as a host material of the light-emitting layer, a hole injection layer material, a hole transport layer material, a hole auxiliary layer material, a light-emitting auxiliary layer material, or an electron blocking layer material.

The organic layer may further comprise at least one layer selected from a hole injection layer, a hole transport layer, a hole auxiliary layer, a light-emitting auxiliary layer, an electron transport layer, an electron injection layer, an interlayer, a hole blocking layer, an electron blocking layer and an electron buffer layer in addition to the light-emitting layer. The organic layer may further comprise an amine-based compound and/or an azine-based compound in addition to the light-emitting material of the present disclosure. Specifically, the hole injection layer, the hole transport layer, the hole auxiliary layer, the light-emitting layer, the light-emitting auxiliary layer, or the electron blocking layer may comprise an amine-based compound, for example, arylamine-based compounds, styrylarylamine-based compounds, etc., as a hole injection material, a hole transport material, a hole auxiliary material, a light-emitting material, a light-emitting auxiliary material, and an electron blocking material. In addition, the electron transport layer, the electron injection layer, the electron buffer layer, and the hole blocking layer may comprise an azine-based compound as an electron transport material, an electron injection material, an electron buffer material, and a hole blocking material. Further, the organic layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising the metal.

The plurality of host materials according to the one embodiment may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The plurality of host materials according to the one embodiment may also be used in an organic electroluminescent device comprising a quantum dot (QD).

The anode and the cathode may be respectively formed with a transparent conductive material, or a transflective or reflective conductive material. The organic electroluminescent device may be a top emission type, a bottom emission type, or a both-sides emission type, depending on the materials forming the first electrode and the second electrode.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. Further, the hole injection layer may be doped with a p-dopant. The electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or electron blocking layer may also be multi-layers, wherein each of the multi-layers may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer is a layer that is placed between the electron transport layer (or electron injection layer) and the light-emitting layer and prevents holes from reaching the cathode, thereby improving the recombination probability of electrons and holes in the light-emitting layer. Multi-layers may be used for the hole blocking layer or the electron transport layer, and a plurality of compounds may be used for each layer. Also, the electron injection layer may be doped with an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In the organic electroluminescent device of the present disclosure, preferably, at least one layer selected from the group consisting of a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiOₓ(1≤X≤2), AlOₓ(1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to produce an organic electroluminescent device having two or more light-emitting layers and emitting white light.

An organic electroluminescent device according to one embodiment may further comprise at least one dopant in the light-emitting layer.

The dopant comprised in the organic electroluminescent device of the present disclosure may be at least one phosphorescent or fluorescent dopant, and is preferably a phosphorescent dopant. The phosphorescent dopant material applied to the organic electroluminescent device of the present disclosure is not particularly limited, but may be preferably selected from the group consisting of the metallated complex compounds of iridium (Ir), osmium (Os), copper (Cu), and platinum (Pt), more preferably selected from the group consisting of ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may be a compound represented by the following formula 101, but is not limited thereto.

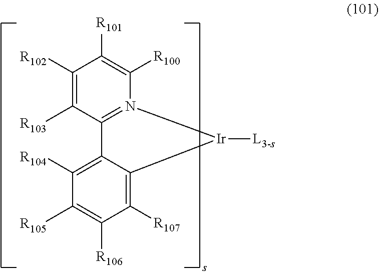

In formula 101,
L is selected from the following structures 1 to 3:

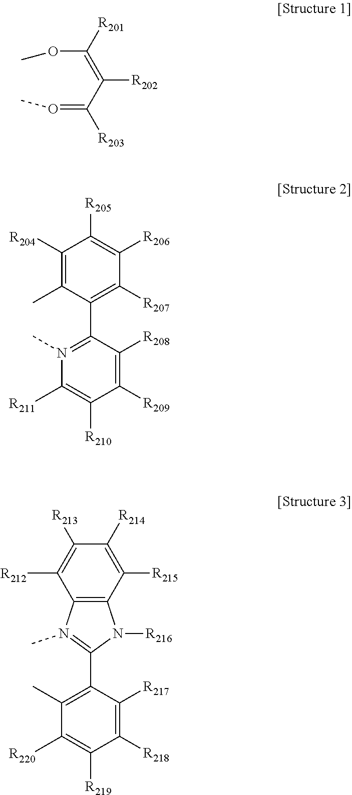

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a substituted or unsubstituted ring(s), e.g., a substituted or unsubstituted, quinoline, benzofuropyridine, benzothienopyridine, indenopyridine, benzofuroquinoline, benzothienoquinoline, or indenoquinoline, together with pyridine;
R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a substituted or unsubstituted ring(s), e.g., a substituted or unsubstituted, naphthalene, fluorene, dibenzothiophene, dibenzofuran, indenopyridine, benzofuropyridine, or benzothienopyridine, together with benzene;
R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent to form a substituted or unsubstituted ring(s); and
s represents an integer of 1 to 3.

The specific examples of the dopant compound are as follows, but are not limited thereto.

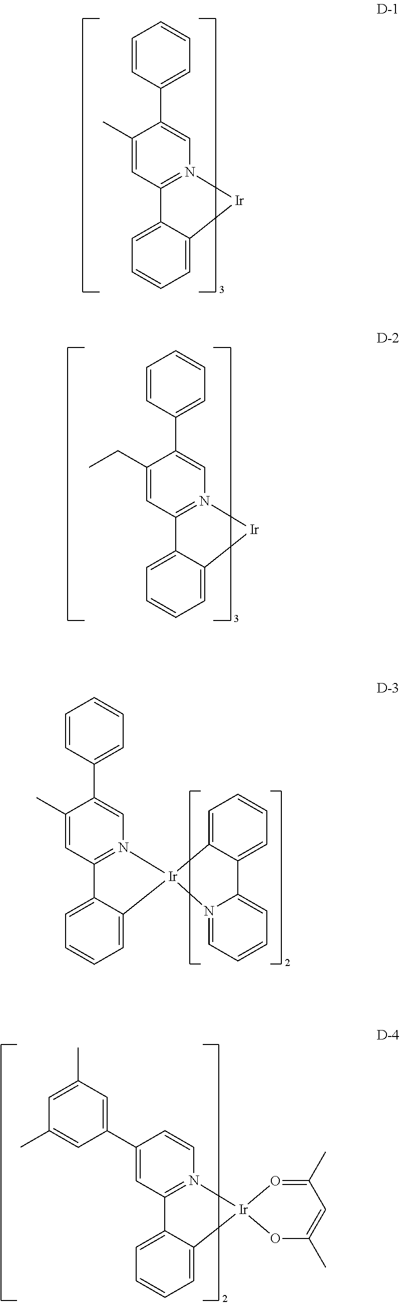

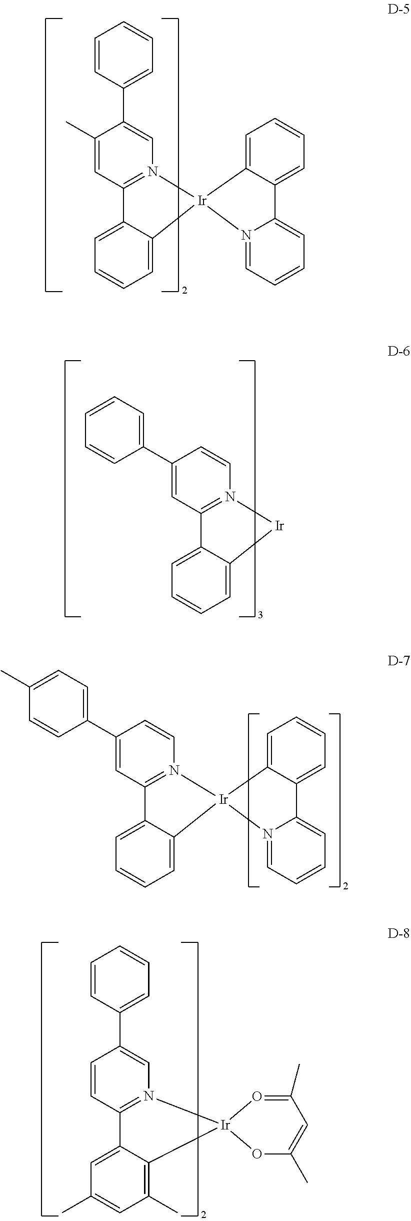

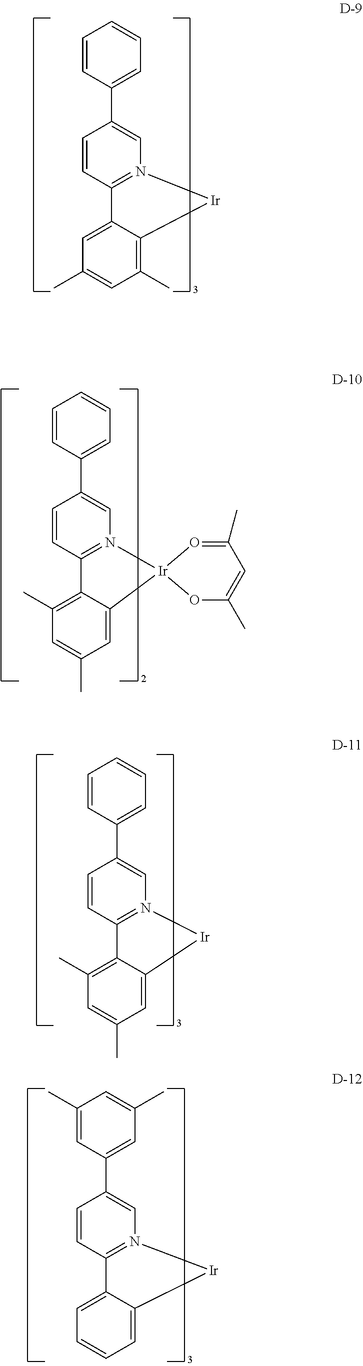

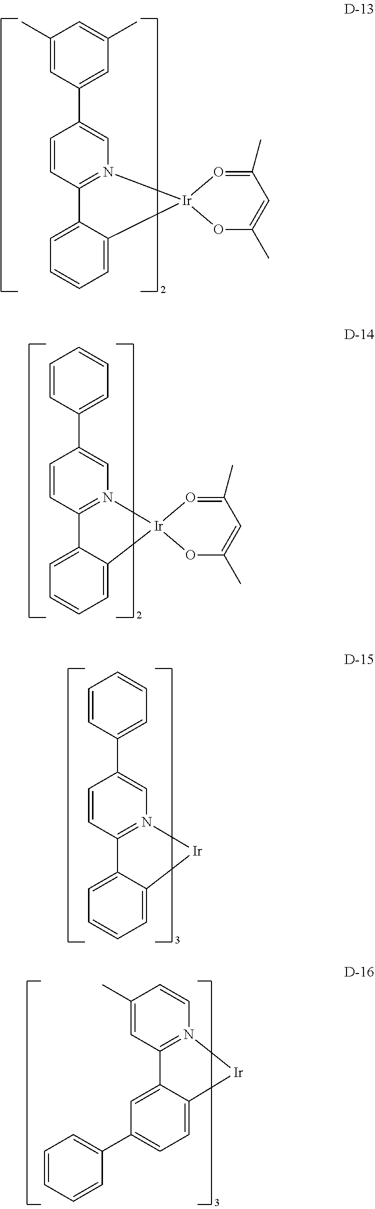

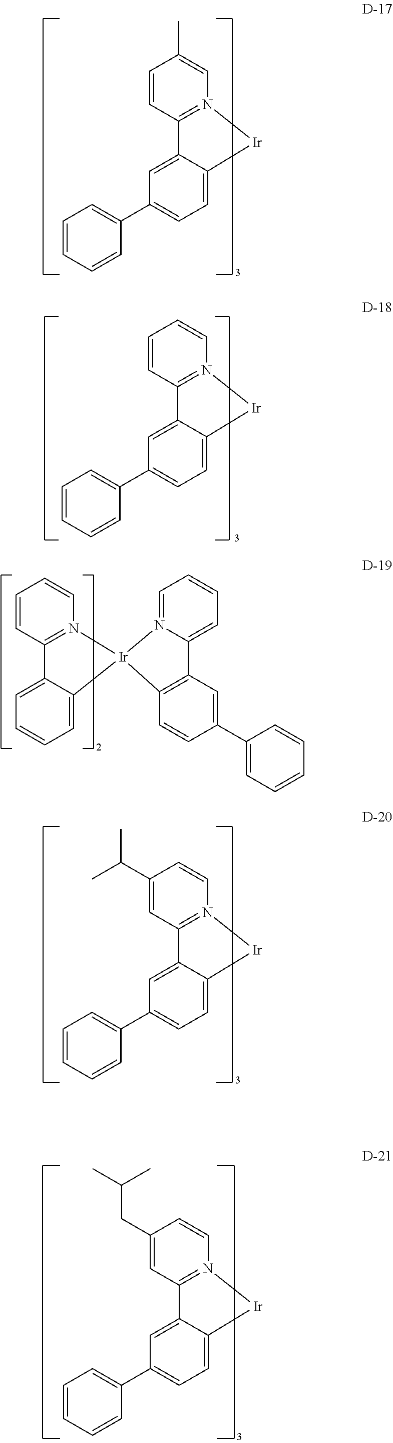

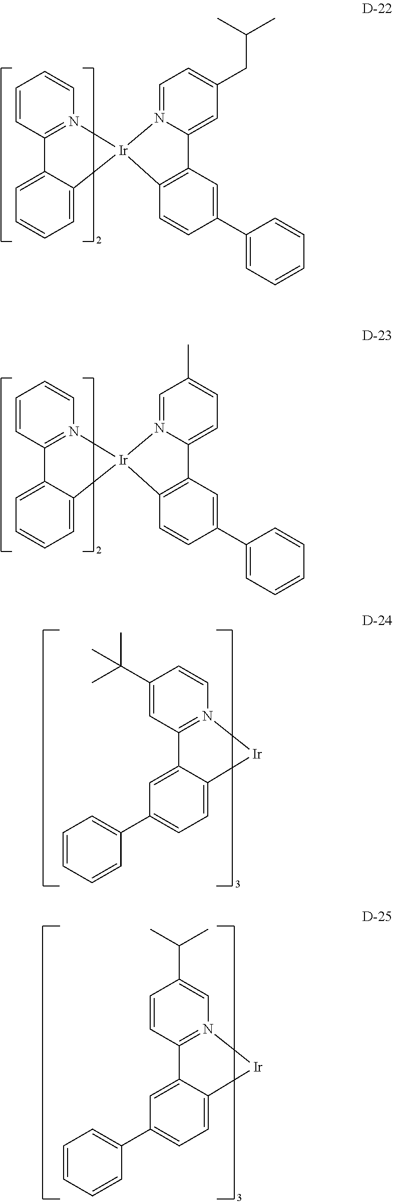

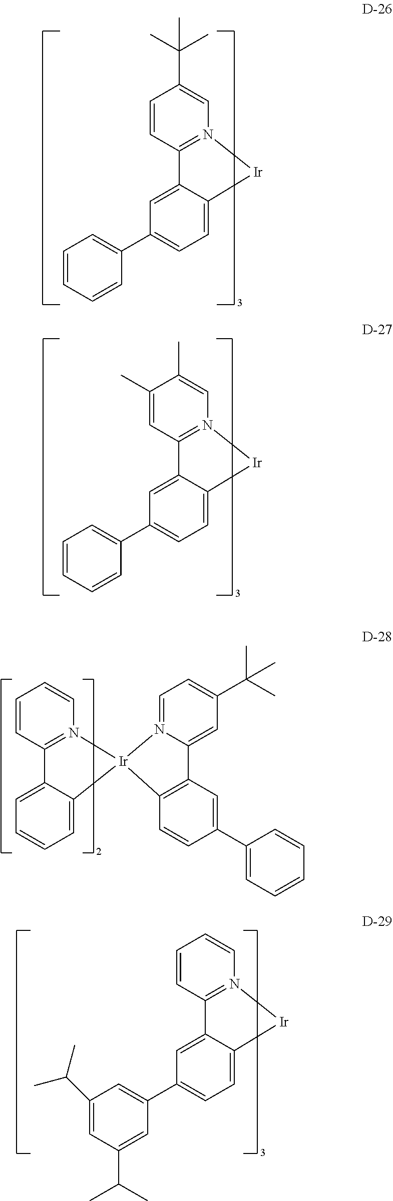

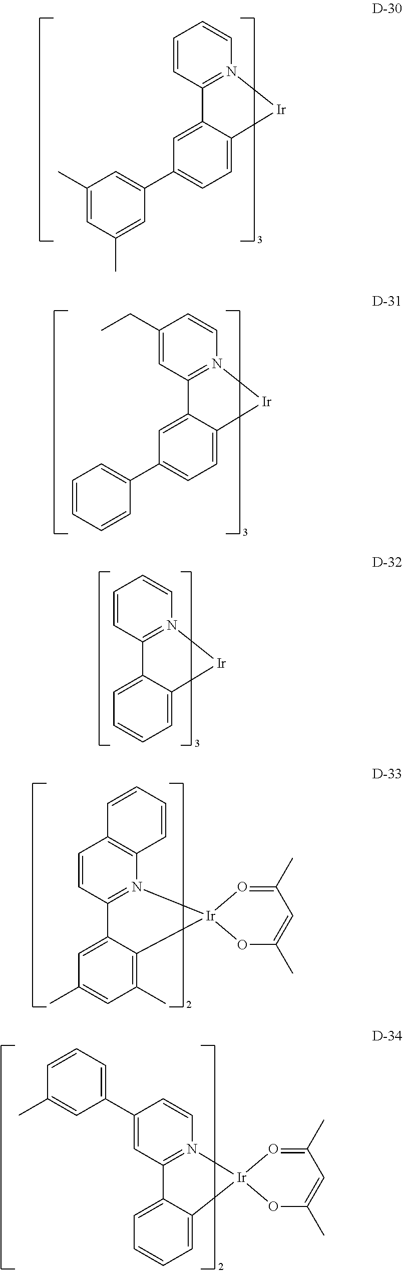

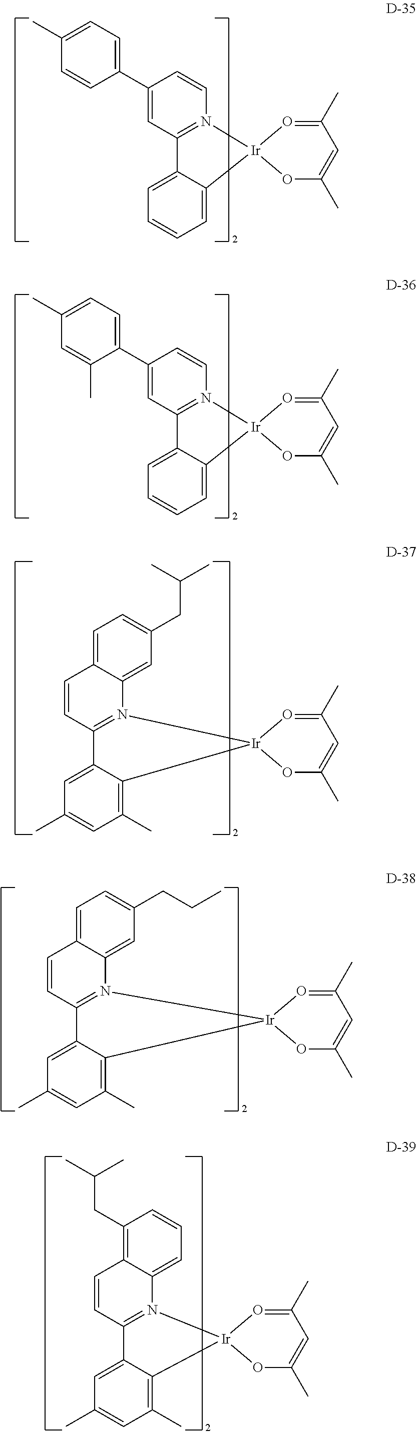

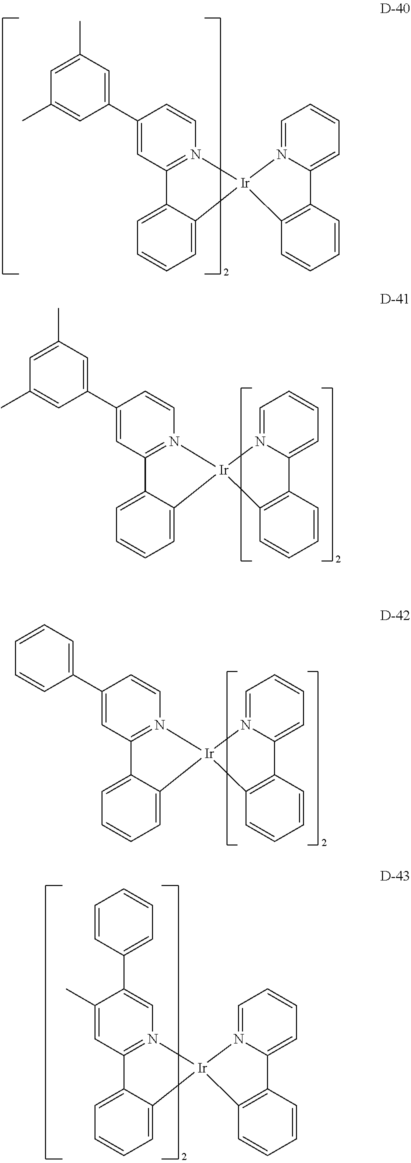

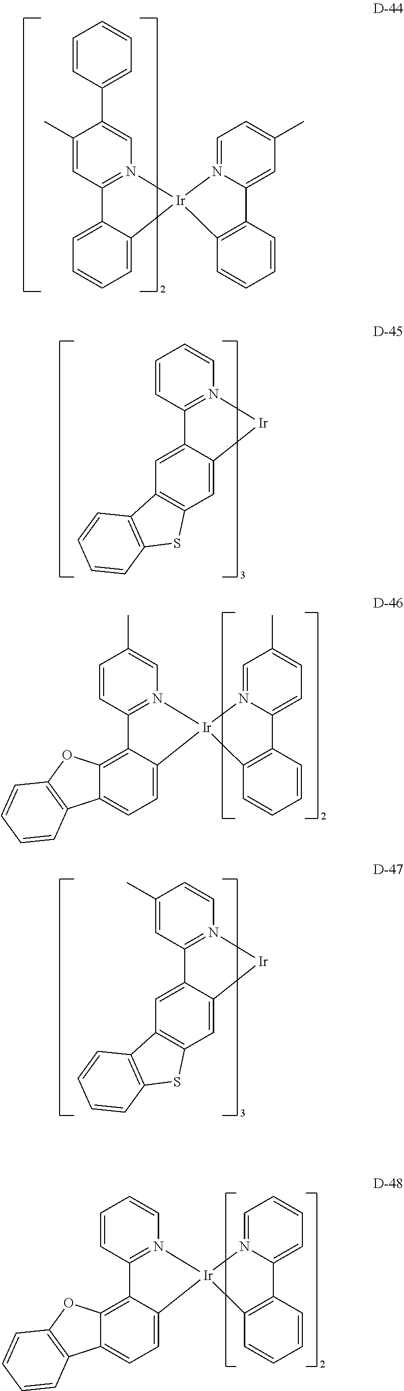

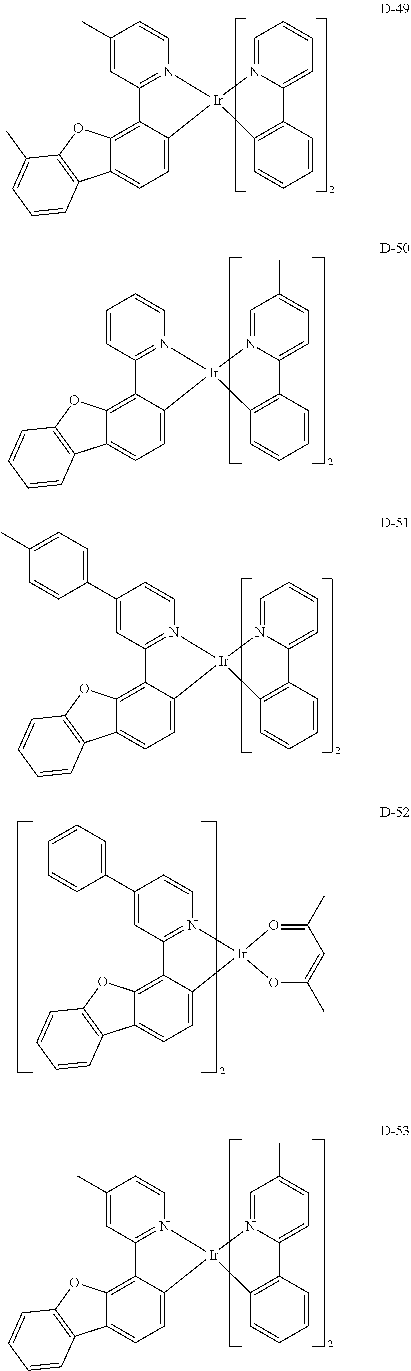

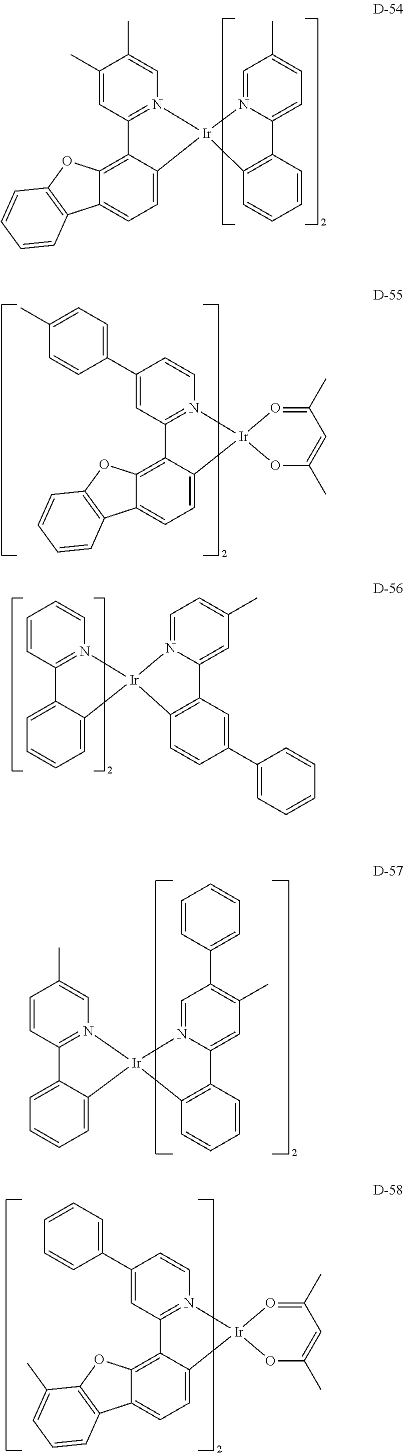

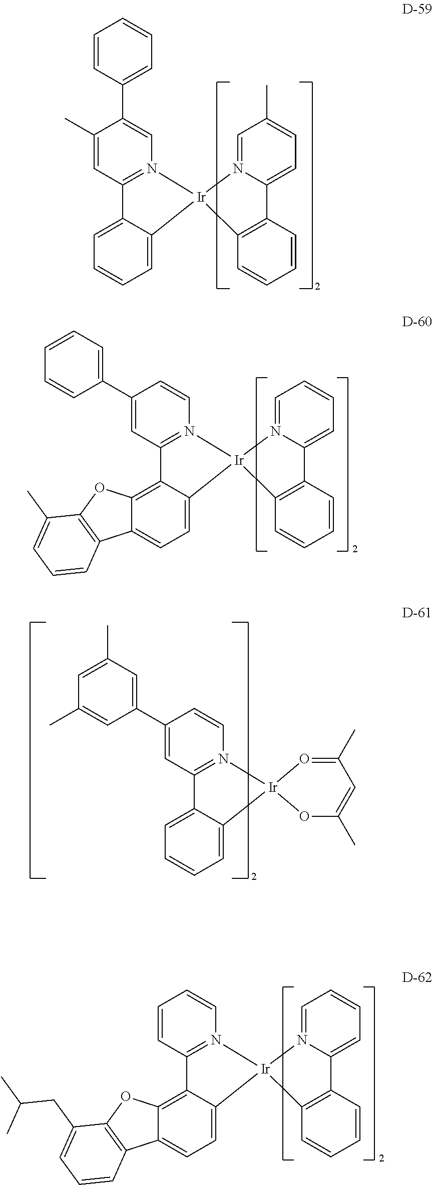

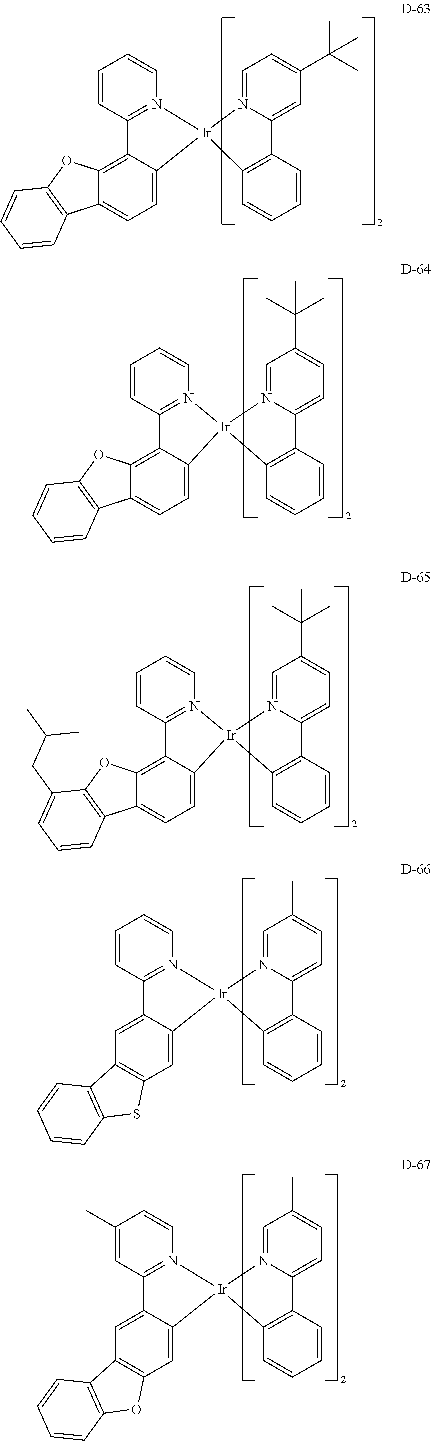

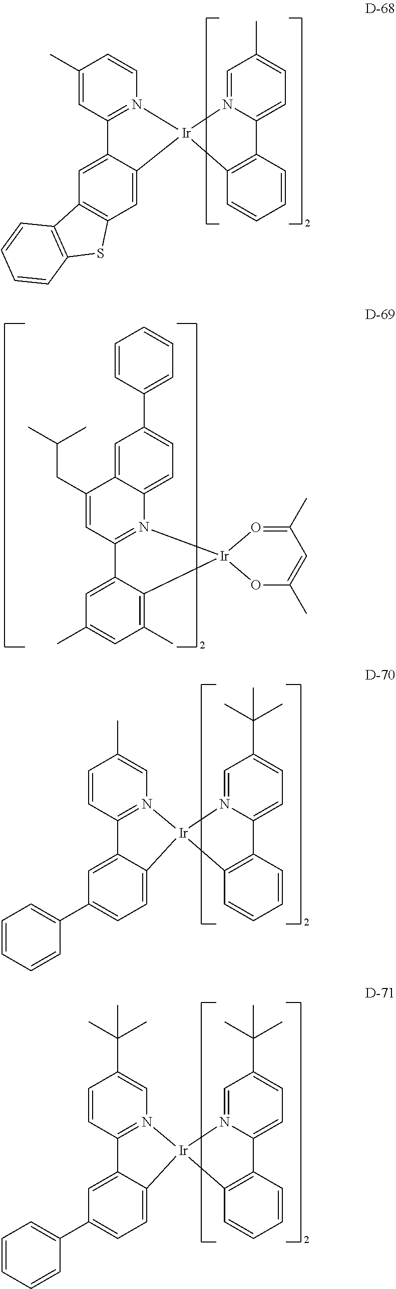

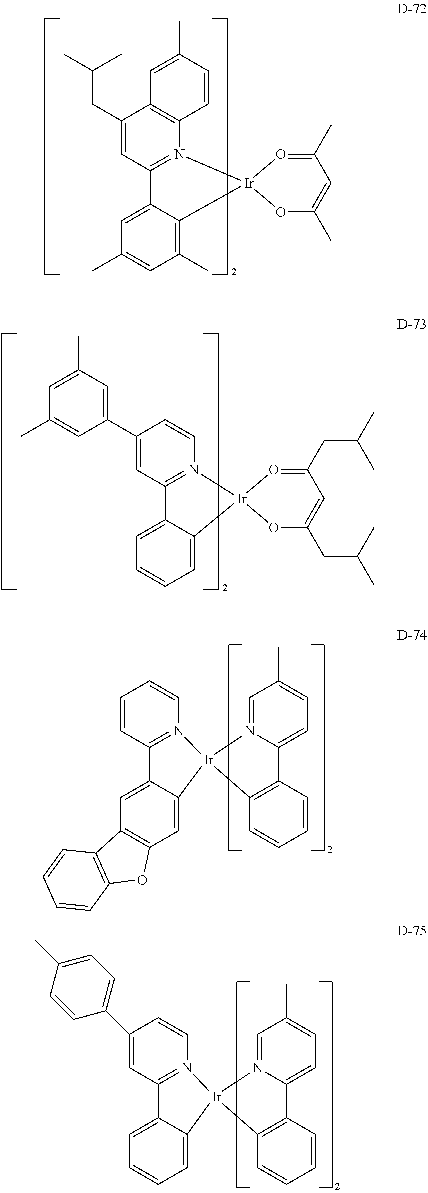

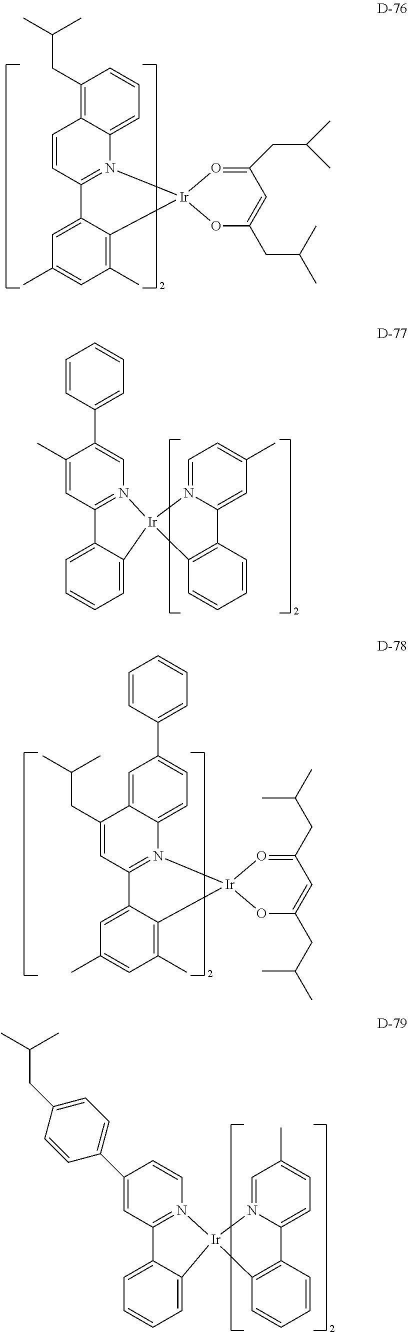

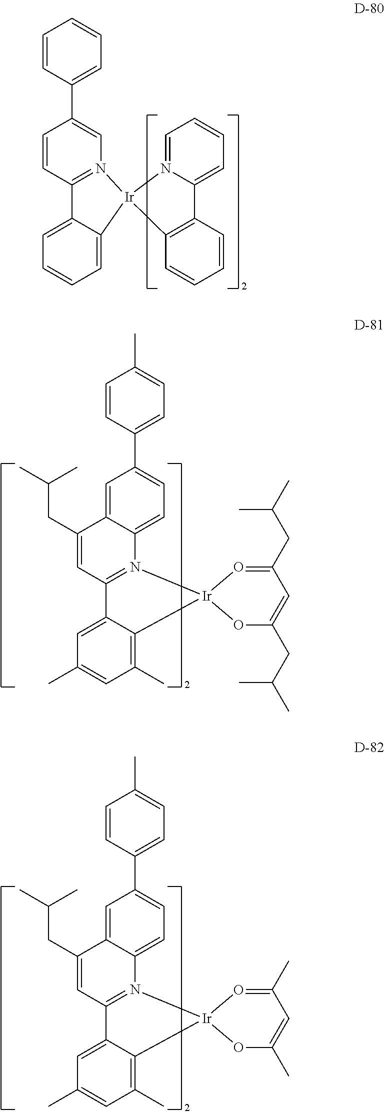

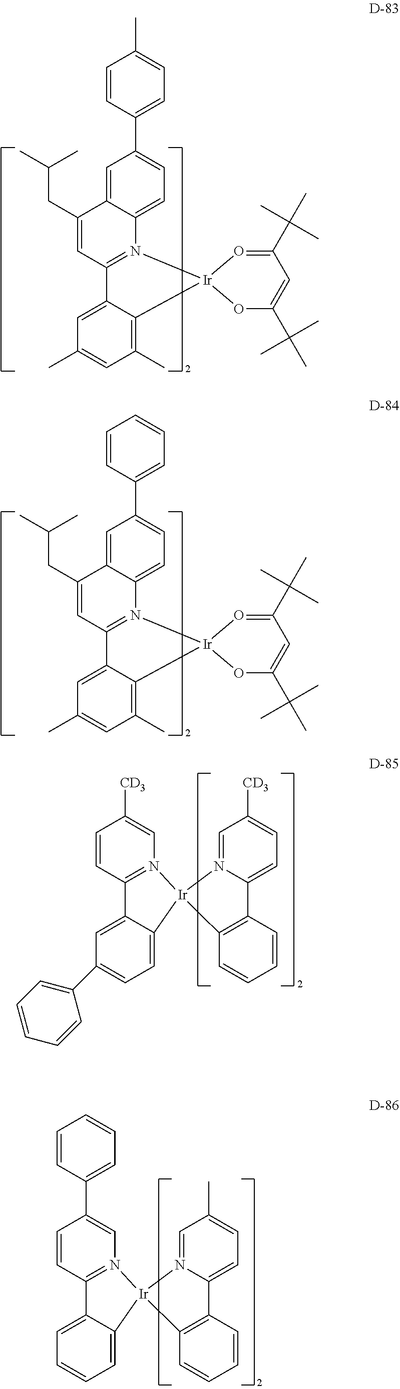

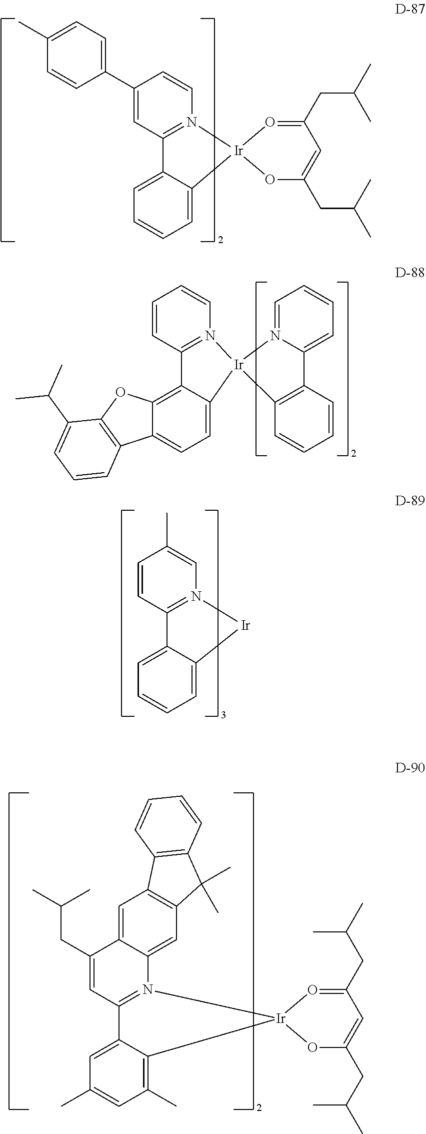

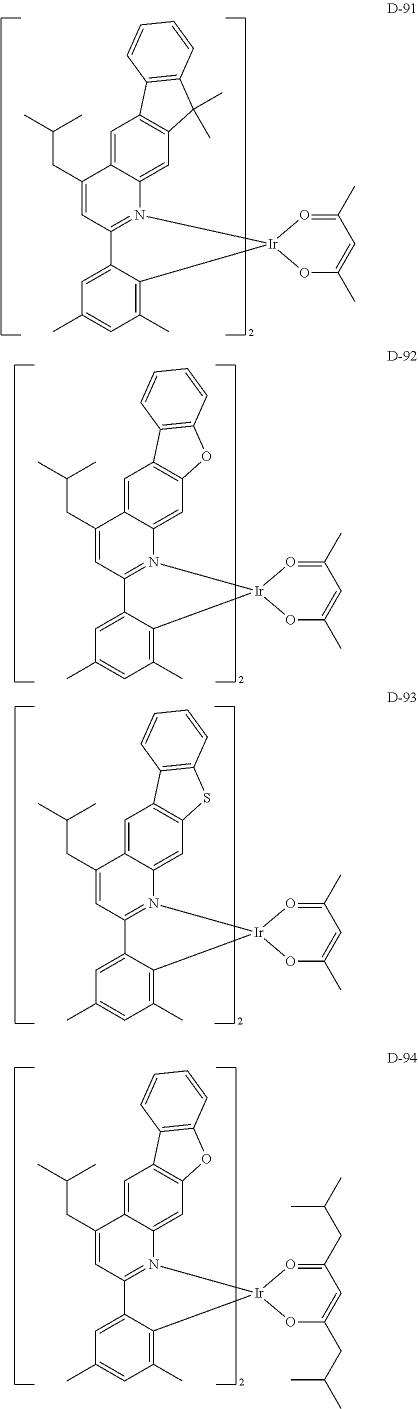

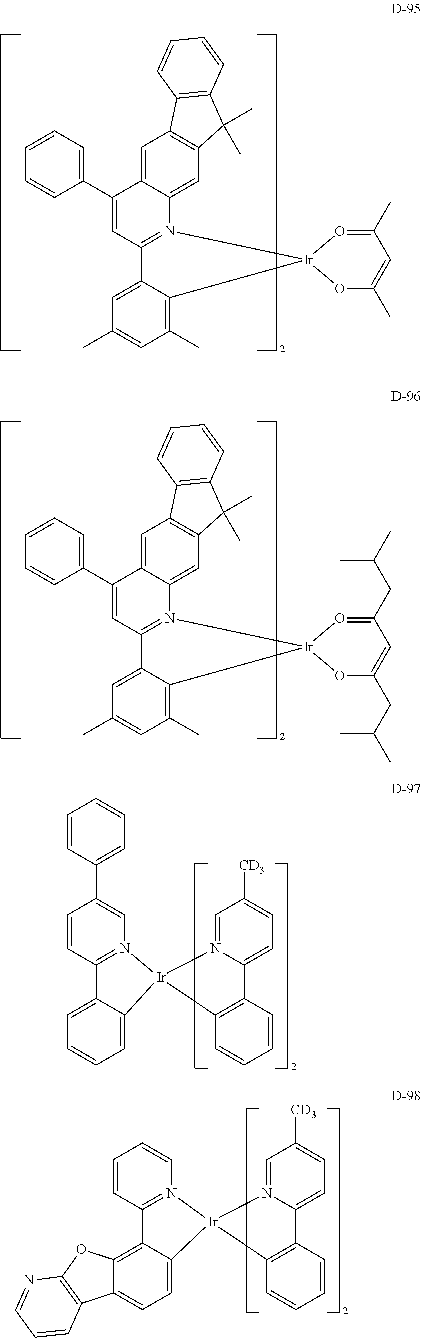

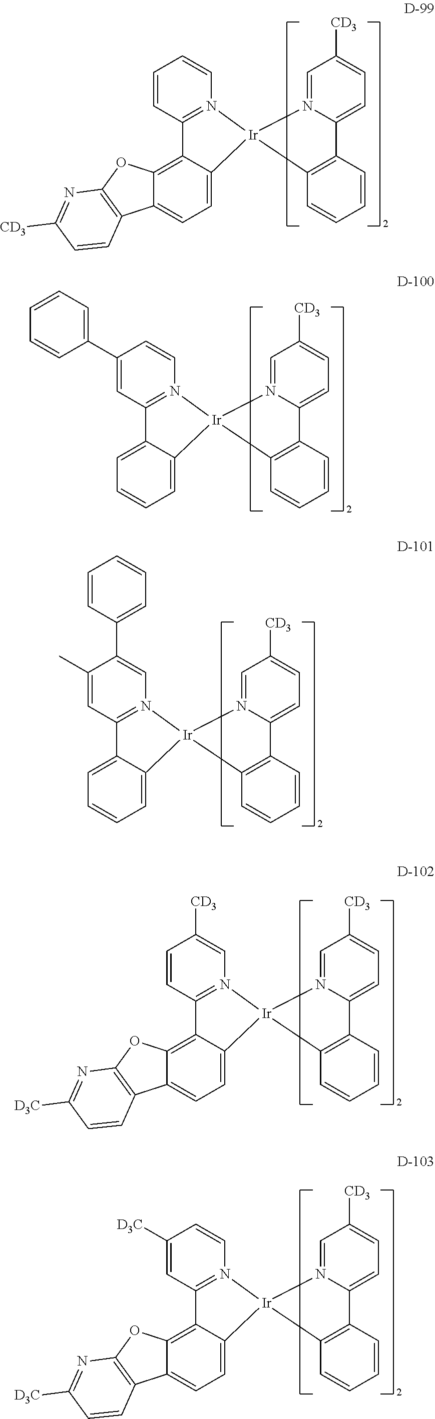

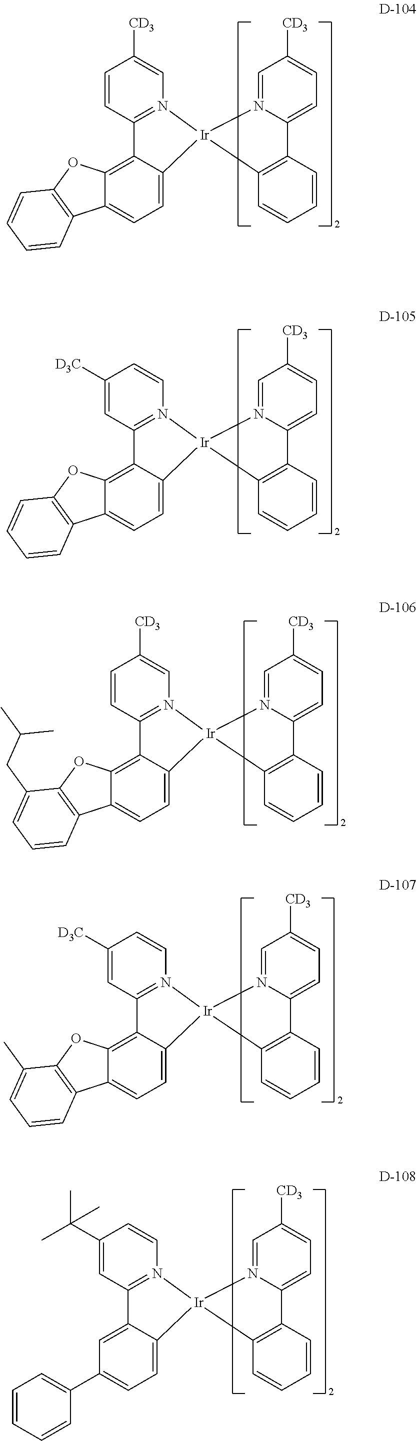

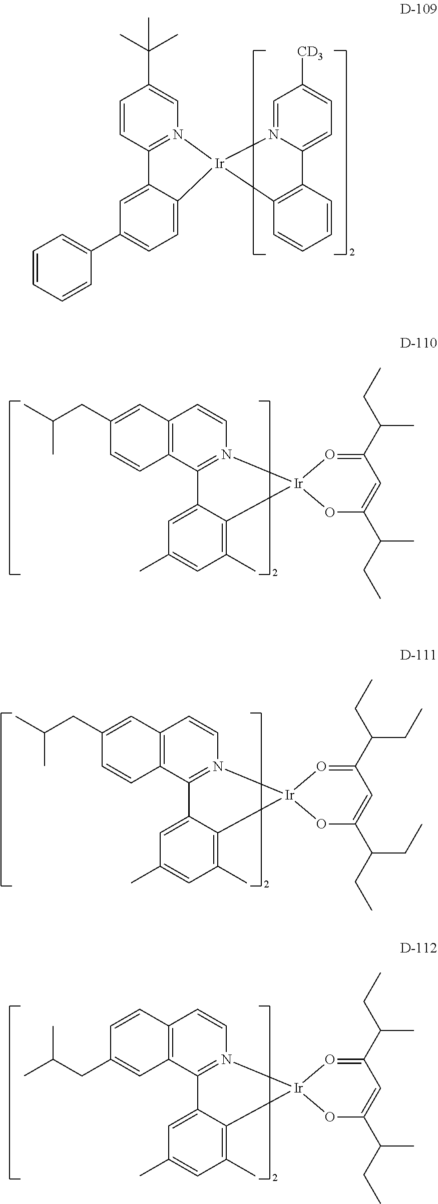

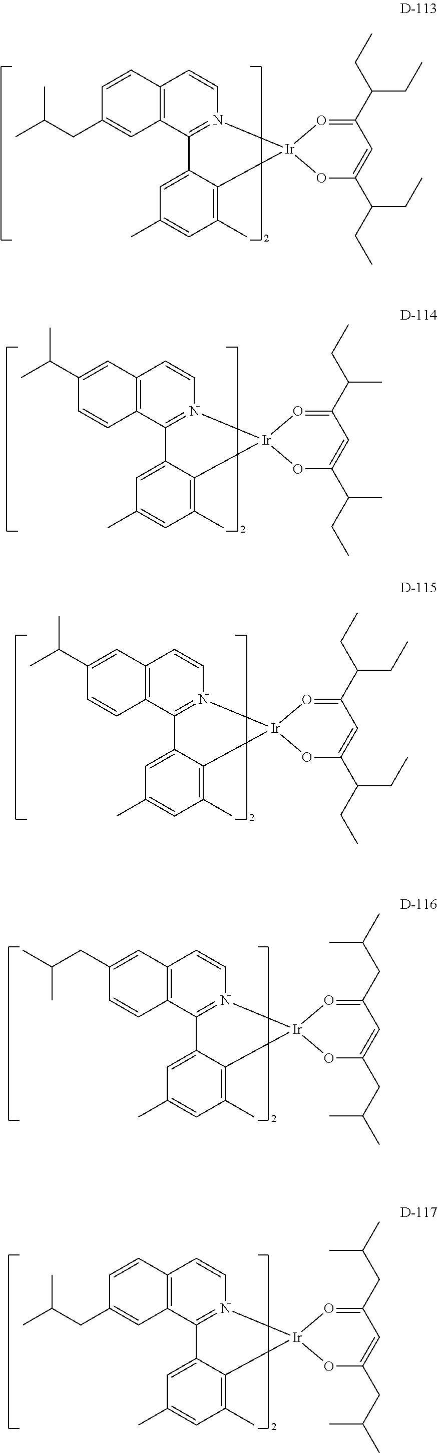

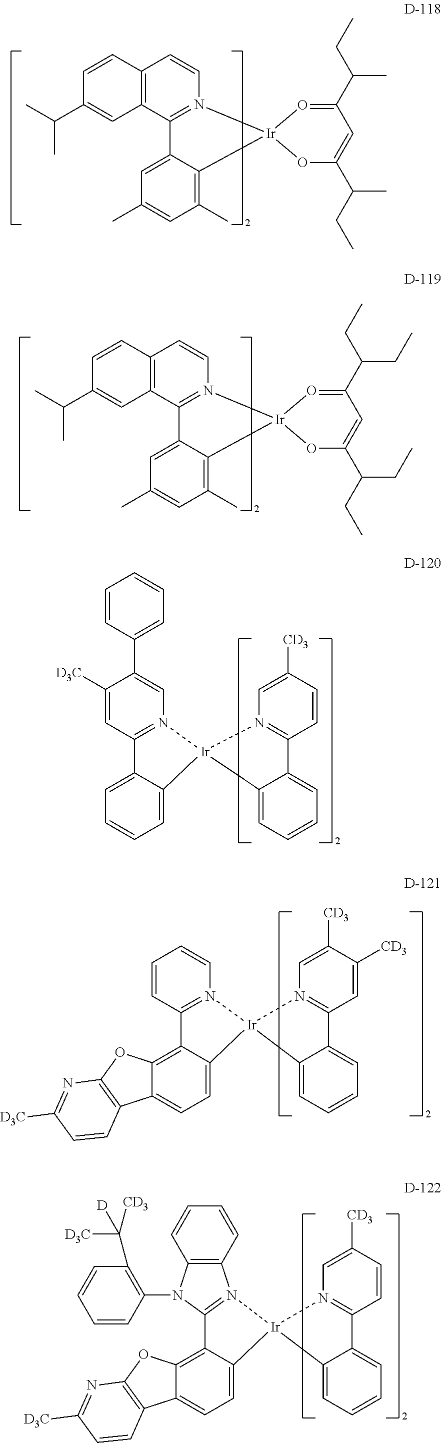

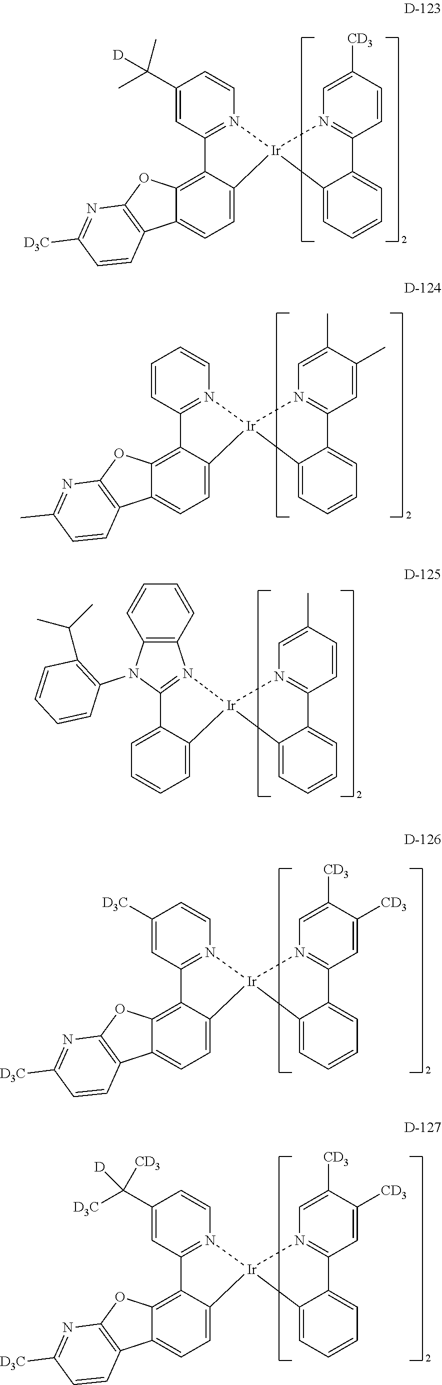

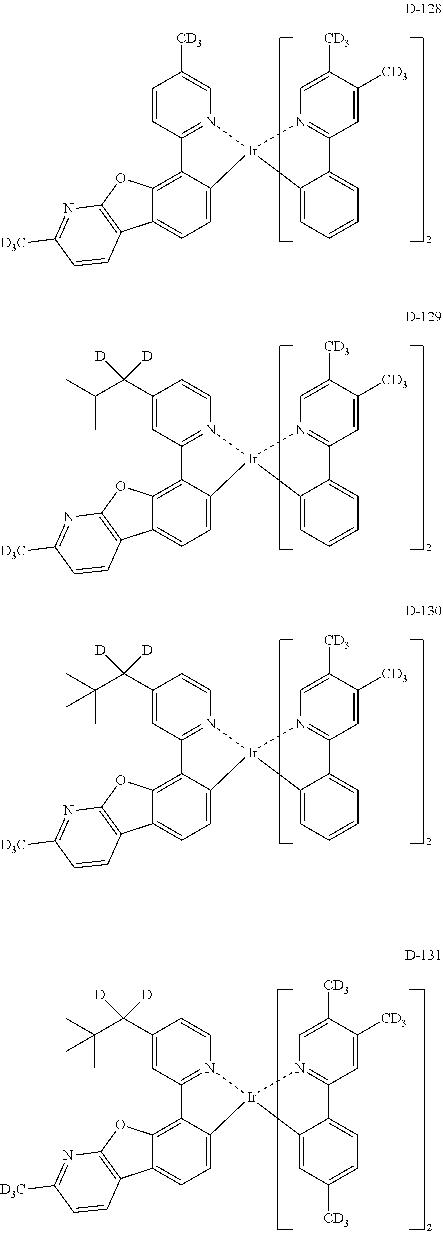

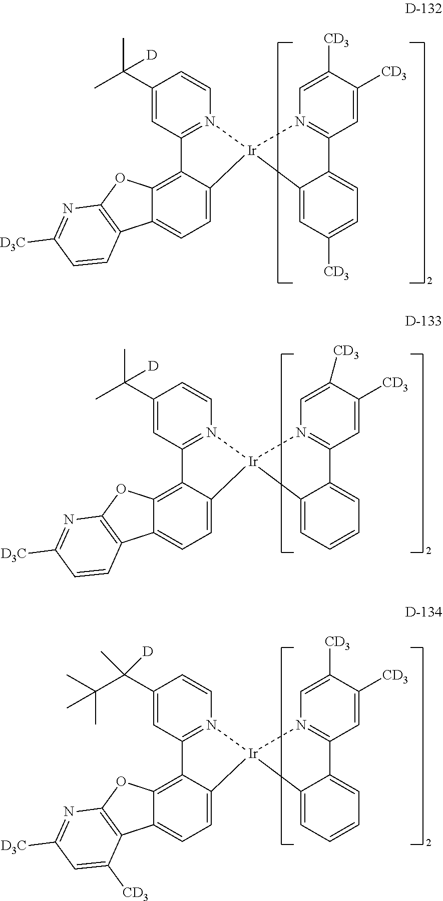

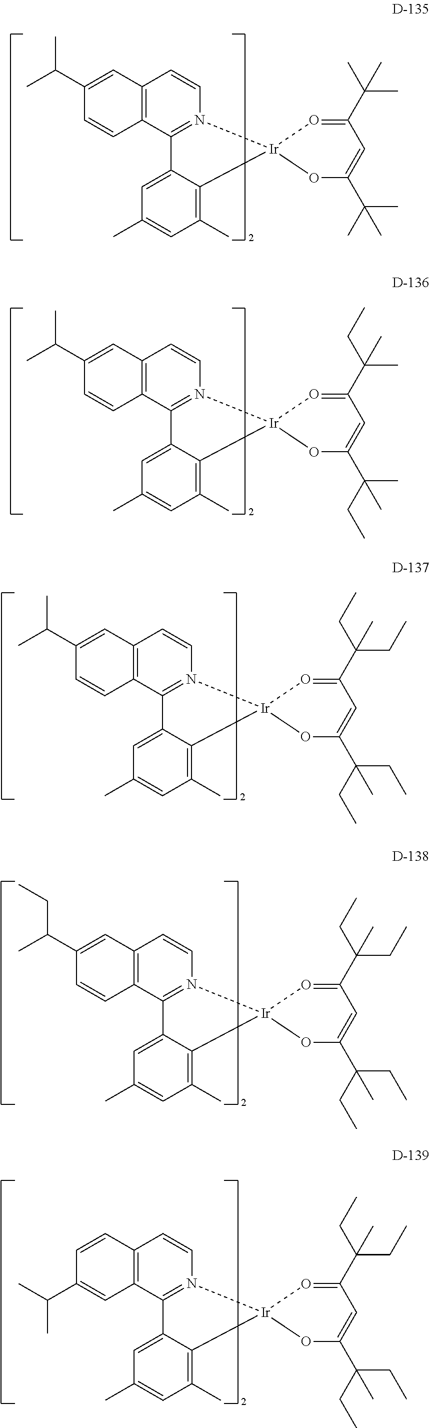

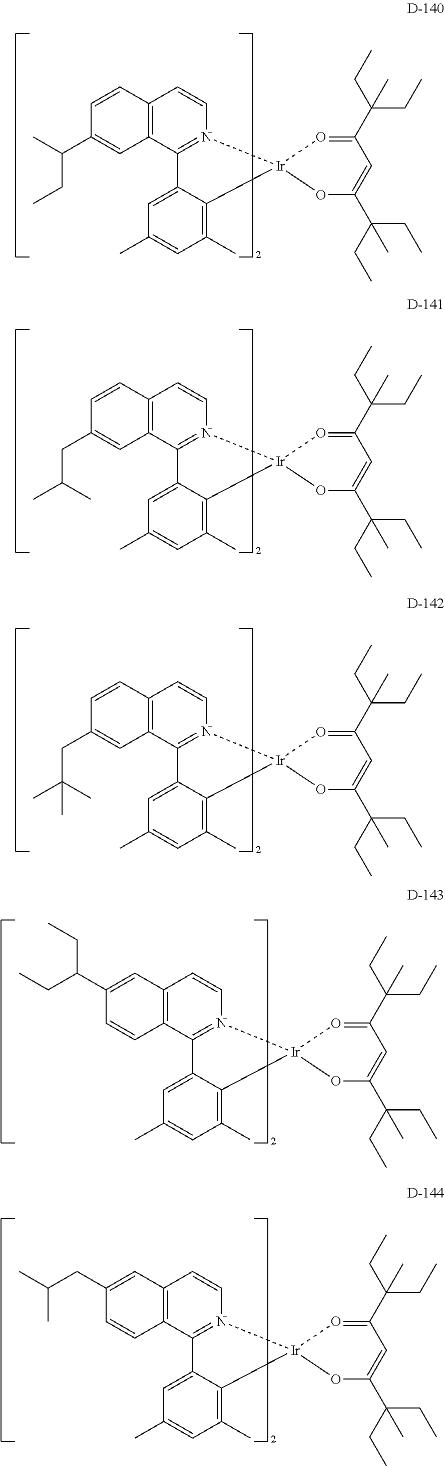

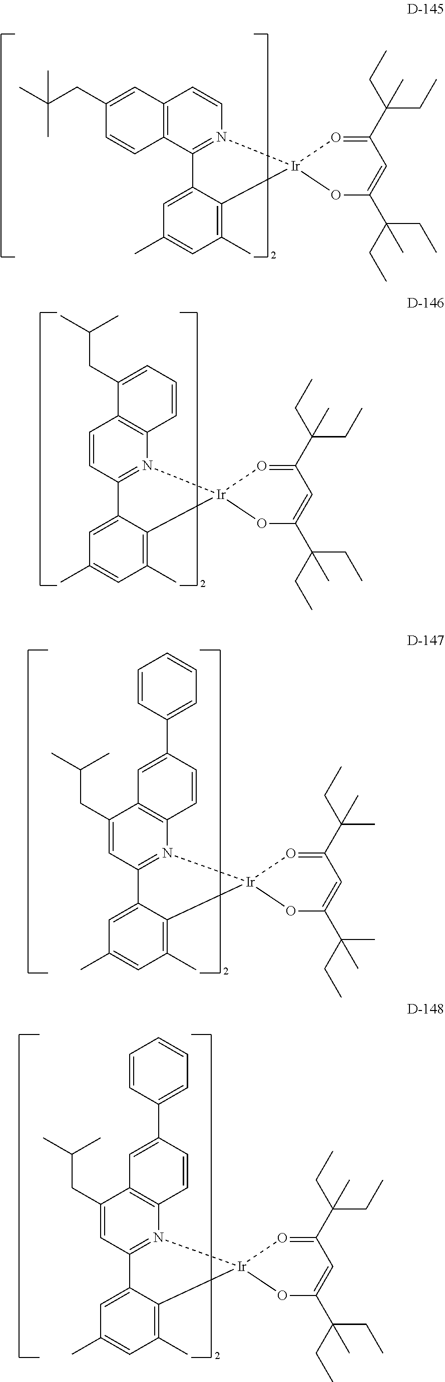

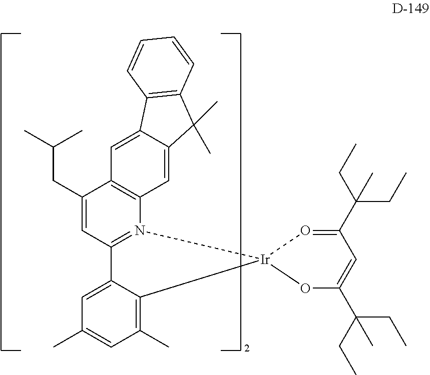

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used. When the organic electroluminescent compound or the first and second host compounds of the present disclosure are used to form a film, a co-evaporation process or a mixture-evaporation process is carried out.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

In addition, it is possible to produce a display system, for example, a display system for smart phones, tablets, notebooks, PCs, TVs, or cars; or a lighting system, for example an outdoor or indoor lighting system, by using the organic electroluminescent device of the present disclosure.

Hereinafter, the preparation method of the compounds according to the present disclosure and the properties thereof, and the light-emitting properties of an organic electroluminescent device (OLED) comprising an organic electroluminescent compound and a plurality of host materials according to the present disclosure will be explained in detail with reference to the representative compounds of the present disclosure. However, the following examples only describe the properties of the OLED comprising the organic electroluminescent compound or the plurality of host materials according to the present disclosure, and the present disclosure is not limited to the following examples.

### Example 1: Preparation of Compound-C-11

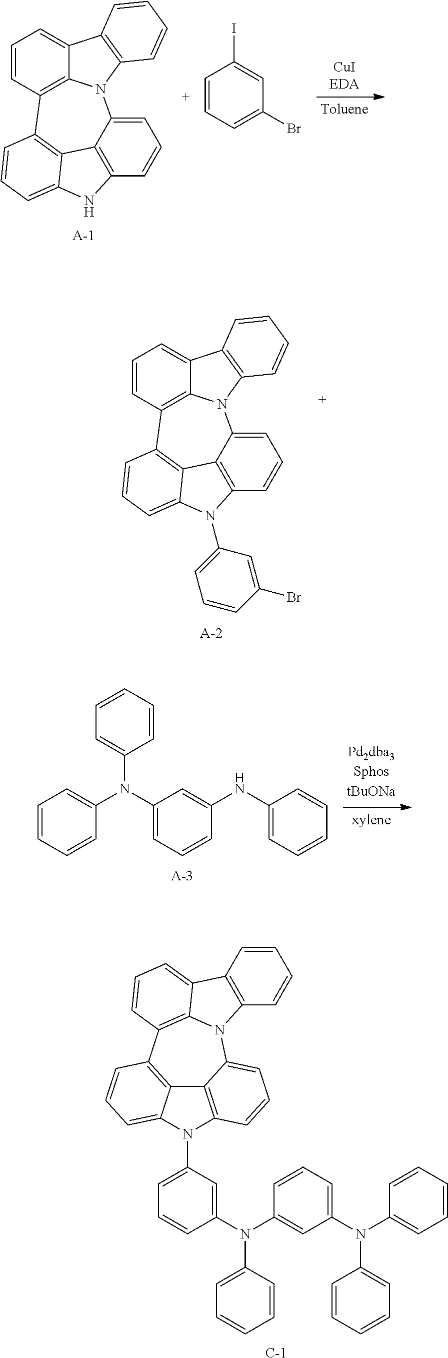

1) Synthesis of Compound A-2

Compound A-1 (4 g, 12 mmol), 4-bromo-2-iodobenzene (9.9 g, 34.8 mmol), CuI (1.15 g, 6 mmol), ethylenediamine (EDA) (1.4 g, 23 mmol), K₃PO₄ (7.6 g, 35.8 mmol), and 60 mL of toluene were added to a flask, and stirred at 130° C. for 12 hours. After the reaction was completed, the reactant was cooled to room temperature, and the organic layer was extracted with ethyl acetate. After the residual moisture was removed using magnesium sulfate, the residue was dried and separated by column chromatography to obtain Compound A-2 (5 g, yield: 85%).

2) Synthesis of Compound C-1

Compound A-2 (3 g, 6.2 mmol), Compound A-3 (2.5 g, 7.4 mmol), Pd₂(dba)₃ (283 mg, 0.31 mmol), S-Phos (203 mg, 0.496 mmol), NaOtBu (1.2 g, 124 mmol) and 126 mL of o-xylene were added to a flask, and stirred at 160° C. for 12 hours. After the reaction was completed, the reactant was cooled to room temperature, and the organic layer was extracted with ethyl acetate. After the residual moisture was removed using magnesium sulfate, the residue was dried and separated by column chromatography to obtain Compound C-1 (2 g, yield: 44%).

| Compound | MW | M.P. |
|---|---|---|
| C-1 | 740.91 | 136.9° C. |

### Example 2: Preparation of Compound C-2

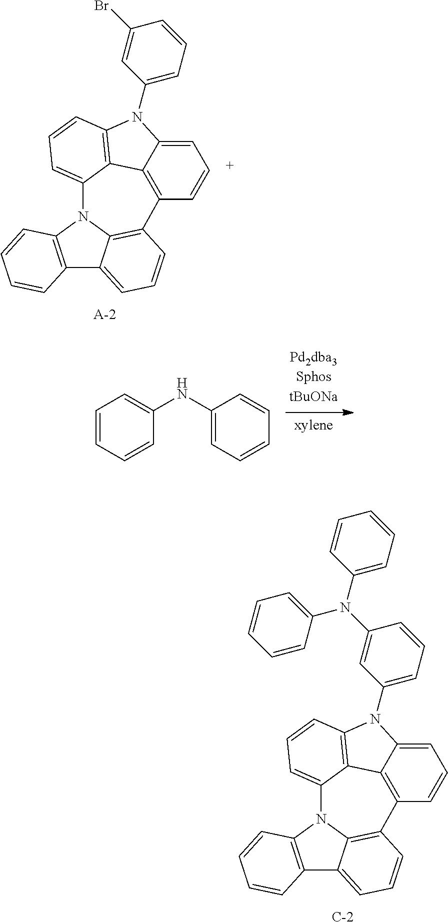

Compound A-2 (2.0 g, 4.121 mol), diphenylamine (0.83 g, 4.944 mmol), NaOtBu (0.79 g, 8.242 mmol), S-Phos (135 mg, 0.329 mmol), and Pd₂(dba)₃ (188 mg, 0.206 mmol) were added to a flask, dissolved in 20 mL of xylene, and stirred under reflux at 130° C. for 12 hours. After the reaction was completed, the organic layer was extracted with ethyl acetate. After the residual moisture was removed using magnesium sulfate, the residue was dried and separated by column chromatography to obtain Compound C-2 (1.4 g, yield: 66%).

| Compound | MW | M.P. |
|---|---|---|
| C-2 | 573.70 | 246.6° C. |

### Example 3: Preparation of Compound E-27

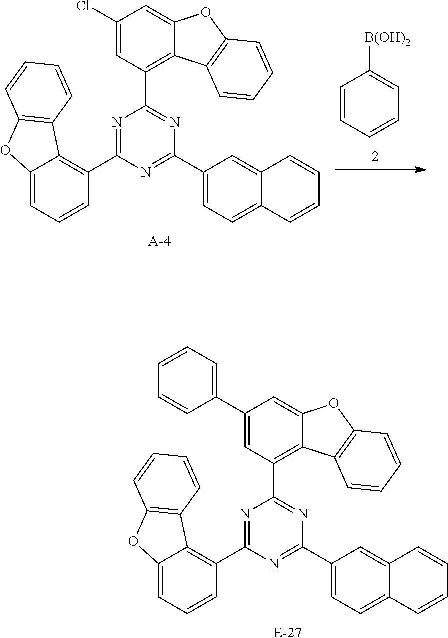 [SMILES: Clc1cc(-c2nc(-c3ccc4ccccc4c3)nc(-c3cccc4oc5ccccc5c34)n2)c2c(c1)oc1ccccc12 ; OB(O)c1ccccc1 ; "A-4" ; c1ccc(-c2cc(-c3nc(-c4ccc5ccccc5c4)nc(-c4cccc5oc6ccccc6c45)n3)c3c(c2)oc2ccccc23)cc1 ; "E-27"]

Compound A-4 (8.3 g, 14.45 mmol), Compound 2 (4.6 g, 36.14 mmol), Pd(OAc)₂ (0.32 g, 1.445 mmol), S-Phos (1.2 g, 2.891 mmol), and Cs₂CO₃ (14.1 g, 43.37 mmol) were added to a flask, dissolved in 80 mL of o-xylene, 20 mL of 1,4-dioxane, and 20 mL of H₂O, and stirred under reflux for 3 hours. After the reaction was completed, the reactant was cooled to room temperature, and MeOH was added thereto. The solid was filtered, dissolved in chlorobenzene, and filtered through silica to obtain Compound E-27 (2.5 g, yield: 28%).

| Compound | MW | M.P. |
|---|---|---|
| E-27 | 615.6 | 211.4° C. |

### Example 4: Preparation of Compound E-43

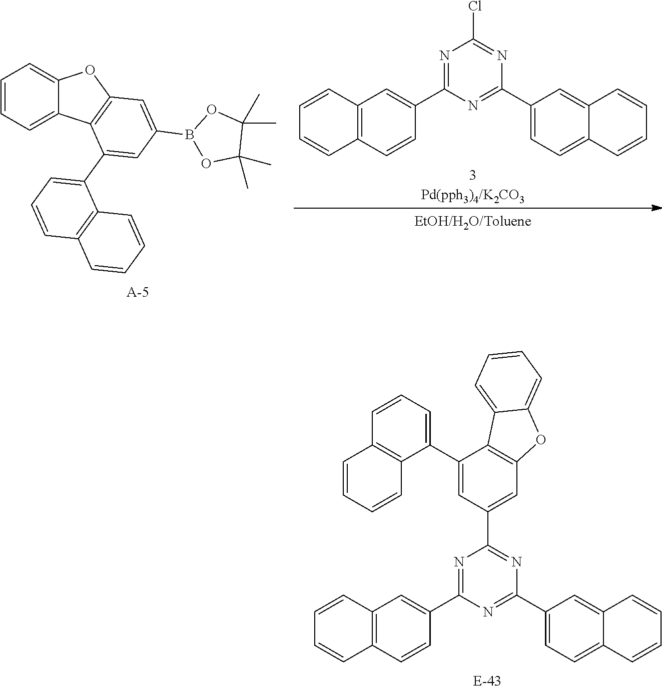

Compound A-5 (15.7 g, 37.35 mmol), Compound 3 (11 g, 29.88 mmol), Pd(pph₃)₄ (2.1 g, 1.867 mmol), and K₂CO₃ (15.4 g, 112.0 mmol) were added to a flask, dissolved in 55 mL of EtOH, 55 mL of H₂O, and 220 mL of toluene, and stirred under reflux for 2 hours. After the reaction was completed, the reactant was cooled to room temperature, and MeOH was added thereto. The solid was filtered, dissolved in chlorobenzene, and filtered through silica to obtain Compound E-43 (4 g, yield: 17%).

| Compound | MW | M.P. |
|---|---|---|
| E-43 | 625.7 | 248.5° C. |

### Device Examples 1 and 2: Producing OLEDs by Co-Depositing the First Host Compound and the Second Host Compound According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and Compound HT-1 was introduced into another cell of the vacuum vapor deposition apparatus. The two materials were evaporated at different rates, and Compound HI-1 was deposited in a doping amount of 3 wt % based on the total amount of Compound HI-1 and Compound HT-1 to form a hole injection layer having a thickness of 10 nm on the ITO substrate. Next, Compound HT-1 was deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: each of the first host compound and the second host compound shown in Table 1 below were introduced into two cells of the vacuum vapor deposition apparatus as hosts, and Compound D-39 was introduced into another cell as a dopant. The two host materials were evaporated at a rate of 1:1 and the dopant material was simultaneously evaporated at a different rate, and the dopant was deposited in a doping amount of 3 wt % based on the total amount of the host and the dopant to form a light-emitting layer having a thickness of 40 nm on the second hole transport layer. Compound ET-1 and Compound EI-1 were evaporated in a weight ratio of 50:50 to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Example 1: Producing an OLED Comprising a Single Host

An OLED was produced in the same manner as in Device Example 1, except that the second host compound shown in Table 1 below was used alone as a host of the light-emitting layer.

The driving voltage, luminous efficiency, and light-emitting color at a luminance of 1,000 nit, and the time taken for luminance to decrease from 100% to 95% at a luminance of 10,000 nit (lifetime; T95) of the OLEDs produced in Device Examples 1 and 2 and Comparative Example 1 are provided in Table 1 below.

TABLE 1

| | First | Second | Driving | Luminous | Light- | Lifetime |
|---|---|---|---|---|---|---|
| | Host | Host | Voltage | Efficiency | Emitting | T95 |
| | Compound | Compound | (V) | (cd/A) | Color | (hr) |
| Device | C-1 | H2-77 | 3.0 | 34.2 | Red | 75 |
| Example 1 | | | | | | |
| Device | C-2 | H2-77 | 2.9 | 32.2 | Red | 48 |
| Example 2 | | | | | | |
| Comparative | — | H2-77 | 3.5 | 31.8 | Red | 18 |
| Example 1 | | | | | | |

As shown in Table 1 above, it can be confirmed that the OLEDs using a plurality of host materials comprising a compound represented by formula 1 of the present disclosure and a compound represented by formula 2 of the present disclosure exhibit low driving voltage, high luminous efficiency, and long lifetime properties, compared to the OLED using the compound represented by formula 2 as a single host material.

### Device Examples 3 and 4: Producing OLEDs by Depositing the Host Compound According to the Present Disclosure

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 was introduced into a cell of the vacuum vapor deposition apparatus, and Compound HT-1 was introduced into another cell of the vacuum vapor deposition apparatus. The two materials were evaporated at different rates, and Compound HI-1 was deposited in a doping amount of 3 wt % based on the total amount of Compound HI-1 and Compound HT-1 to form a hole injection layer having a thickness of 10 nm on the ITO substrate. Next, Compound HT-1 was deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: the host compound shown in Table 2 below was introduced into the cell of the vacuum vapor deposition apparatus as a host, and Compound D-39 was introduced into another cell as a dopant. The dopant material was evaporated at a different rate, and the dopant was deposited in a doping amount of 3 wt % based on the total amount of the host and the dopant to form a light-emitting layer having a thickness of 40 nm on the second hole transport layer. Compound ET-1 and Compound EI-1 were evaporated in a weight ratio of 50:50 to form an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Example 2: Producing an OLED Comprising a Comparative Compound as a Host

An OLED was produced in the same manner as in Device Example 3, except that the host compound shown in Table 2 below was used as a host of the light-emitting layer.

The driving voltage, luminous efficiency, and light-emitting color at a luminance of 1,000 nit, and the time taken for luminance to decrease from 100% to 95% at a luminance of 10,000 nit (lifetime; T95) of the OLEDs produced in Device Examples 3 and 4 and Comparative Example 2 are provided in Table 2 below.

TABLE 2

| | | Driving | Luminous | Light- | |
|---|---|---|---|---|---|
| | | Voltage | Efficiency | Emitting | Lifetime |
| | Host | (V) | (cd/A) | Color | T95 (hr) |
| Device Example 3 | E-27 | 3.2 | 31.0 | Red | 34 |
| Device Example 4 | E-45 | 2.8 | 30.3 | Red | 54 |
| Comparative | T-1 | 3.7 | 25.0 | Red | 11 |
| Example 2 | | | | | |

As shown in Table 2 above, it can be confirmed that the OLEDs using a compound represented by any one of formulas 2′-1 to 2′-4 of the present disclosure as a host material exhibit low driving voltage, high luminous efficiency, and long lifetime properties, compared to the OLED comprising the comparative compound as a host material.

The compounds used in the Device Examples and the Comparative Examples are shown in Table 3 below.

TABLE 3

| | |
|---|---|
| Hole Injection Layer/ Hole Transport Layer | 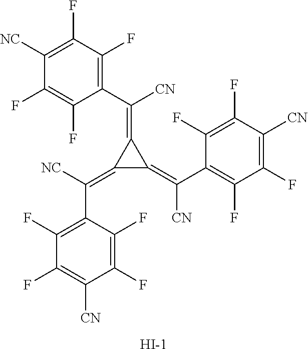 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 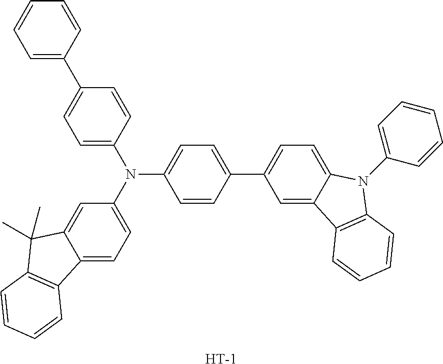 |
| | 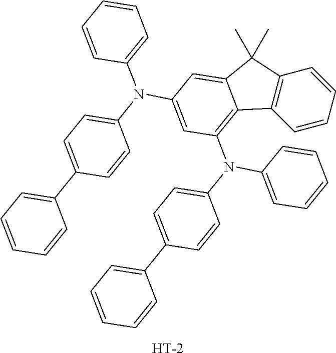 |
| Light-Emitting Layer | 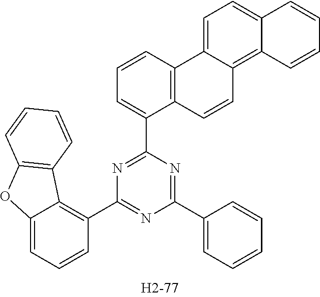 [SMILES: c1ccc(-c2nc(-c3cccc4c3ccc3c5ccccc5ccc43)nc(-c3cccc4oc5ccccc5c34)n2)cc1] |
| | 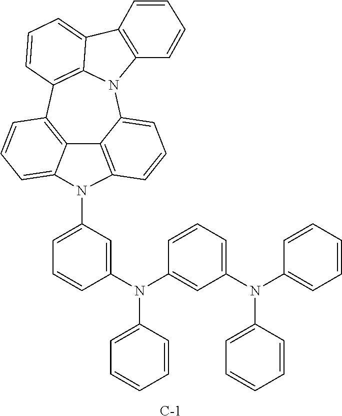 |
| | 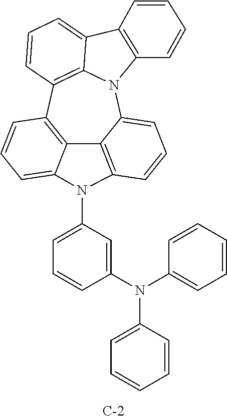 |
| | 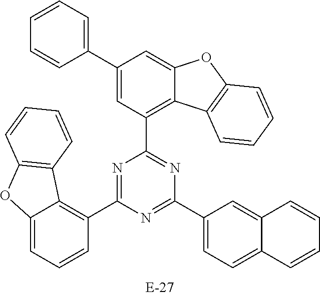 [SMILES: c1ccc(-c2cc(-c3nc(-c4ccc5ccccc5c4)nc(-c4cccc5oc6ccccc6c45)n3)c3c(c2)oc2ccccc23)cc1] |
| | 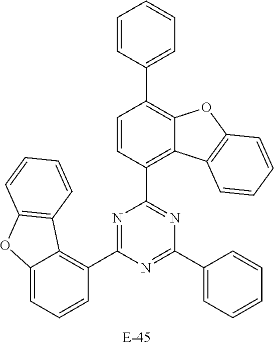 [SMILES: c1ccc(-c2nc(-c3cccc4oc5ccccc5c34)nc(-c3ccc(-c4ccccc4)c4oc5ccccc5c34)n2)cc1] |
| | 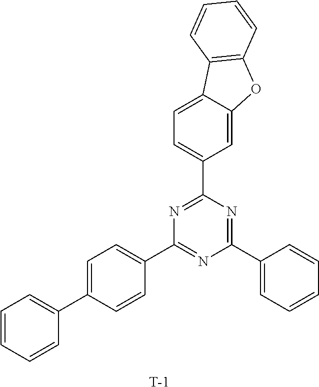 |
| | 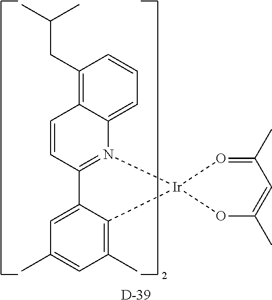 |
| Electron Transport Layer/ Electron Injection Layer | 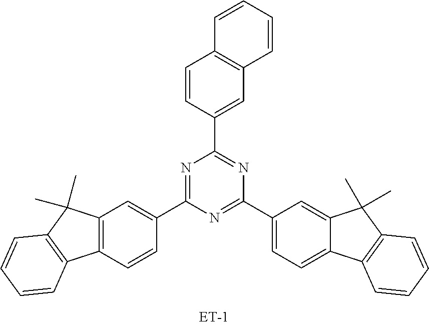 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 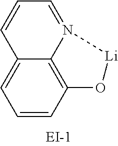 |

1. A plurality of host materials comprising at least one first host compound and at least one second host compound, wherein the first host compound is represented by the following formula 1, and the second host compound is represented by the following formula 2: 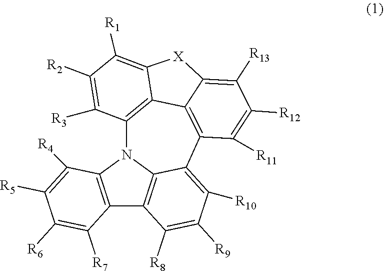
in formula 1,
X represents —O—, —S—, —N(R)—, C(R′)(R″)— or —Se—;
R, R′, and R″, each independently, represent a substituted or unsubstituted (C1-C30)alkyl, a substituted or unsubstituted (C6-C30)aryl, or a substituted or unsubstituted (3- to 30-membered)heteroaryl, -L₁-N—(Ar₁)(Ar₂) or -L₂-N(Ar₃)-L₃-N—(Ar₄)(Ar₅); or R′ and R″ may be linked to each other to form a ring(s), and R′ and R″ may be the same or different;
R₁ to R₁₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), -L₁-N—(Ar₁)(Ar₂) or -L₂-N(Ar₃)-L₃-N—(Ar₄)(Ar₅); or may be linked to an adjacent substituent(s) to form a ring(s);
with the proviso that at least one of R, R′, R″, and R₁ to R₁₃ represents -L₁-N—(Ar₁)(Ar₂) or -L₂-N(Ar₃)-L₃-N—(Ar₄)(Ar₅);
L₁ and L₂, each independently, represent a single bond, a substituted or unsubstituted (C6-C30)arylene, or a substituted or unsubstituted (3- to 30-membered)heteroarylene;
L₃ represents a substituted or unsubstituted (C6-C30)arylene, or a substituted or unsubstituted (3- to 30-membered)heteroarylene; and
Ar₁ to Ar₅, each independently, represent a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl; 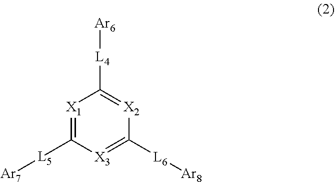
in formula 2,
X₁ to X₃, each independently, represent —N═ or —C(R₂₀)═, with the proviso that at least one of X₁ to X₃ represents N;
R₂₀ represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or a substituted or unsubstituted fused ring group of a (C3-C30) aliphatic ring(s) and a (C6-C30) aromatic ring(s);
L₄ to L₆, each independently, represent a single bond, a substituted or unsubstituted (C6-C30)arylene, a substituted or unsubstituted (C3-C30)cycloalkylene, or a substituted or unsubstituted (3- to 30-membered)heteroarylene;
Ar₆ to Ar₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or *—N—(R₂₁)(R₂₂); or may be linked to an adjacent substituent(s) to form a ring(s); with the proviso that at least one of Ar₆ to Ar₈ represents a substituted or unsubstituted (C6-C30)aryl or a substituted or unsubstituted (3- to 30-membered)heteroaryl; and
R₂₁ and R₂₂, each independently, represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl.

2. The plurality of host materials according to claim 1, wherein the substituent(s) of the substituted alkyl, the substituted alkenyl, the substituted aryl, the substituted arylene, the substituted heteroaryl, the substituted heteroarylene, the substituted cycloalkyl, the substituted cycloalkylene, the substituted alkoxy, the substituted trialkylsilyl, the substituted dialkylarylsilyl, the substituted alkyldiarylsilyl, the substituted triarylsilyl, and the substituted fused ring group of a aliphatic ring(s) and a aromatic ring(s), each independently, are at least one selected from the group consisting of deuterium; a halogen; a cyano; a carboxyl; a nitro; a hydroxyl; a phosphine oxide; a (C1-C30)alkyl; a halo(C1-C30)alkyl; a (C2-C30)alkenyl unsubstituted or substituted with a (C6-C30)aryl(s); a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of a (C1-C30)alkyl(s) and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a substituted or unsubstituted mono- or di-(C6-C30)arylamimo; a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of host materials according to claim 1, wherein formula 1 is represented by at least one of the following formulas 1-1 to 1-28: 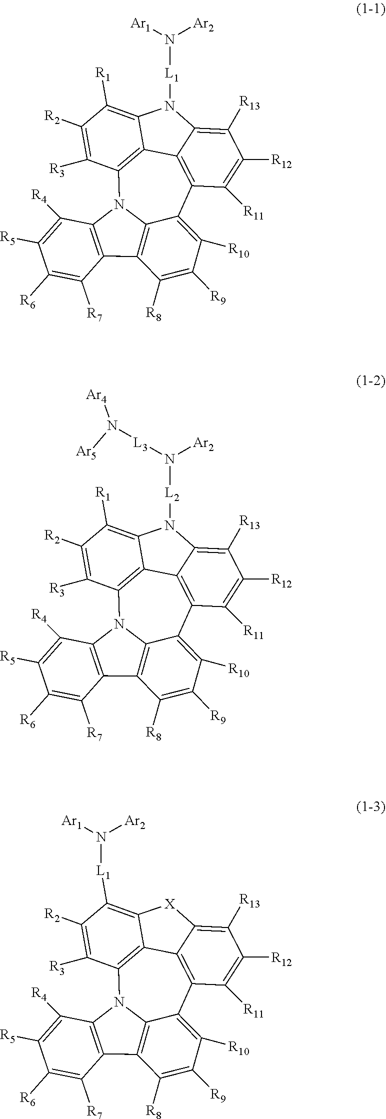 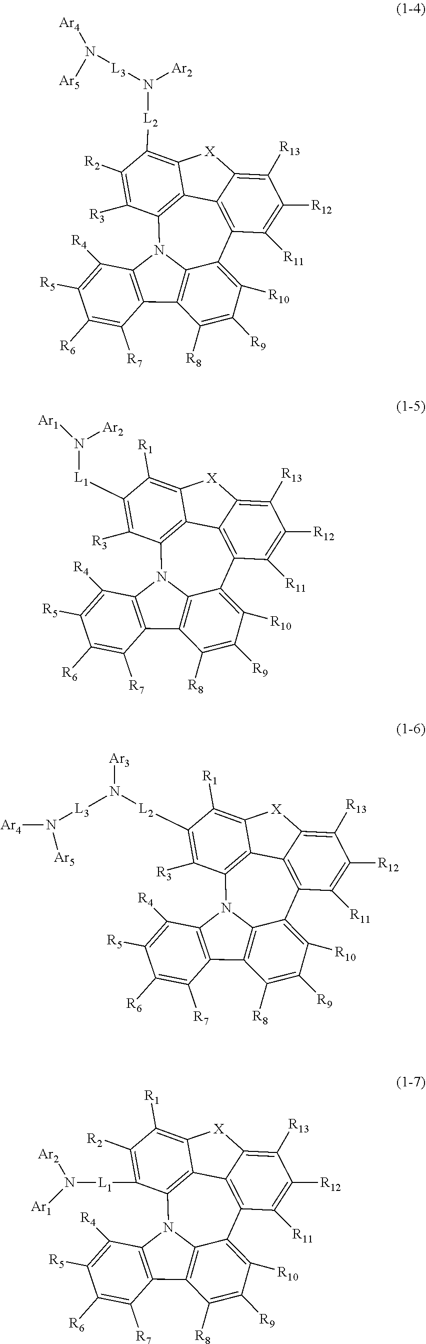 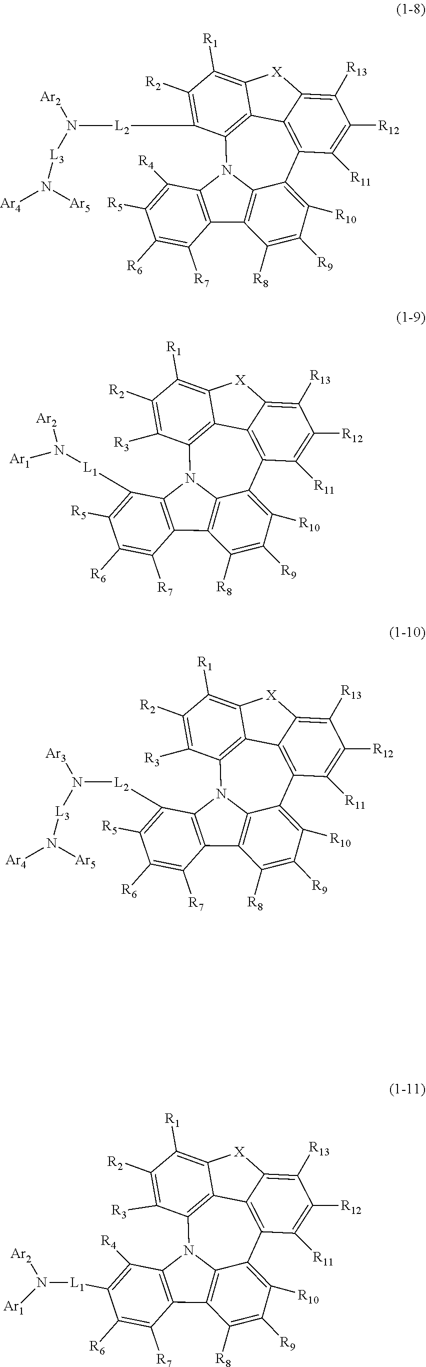 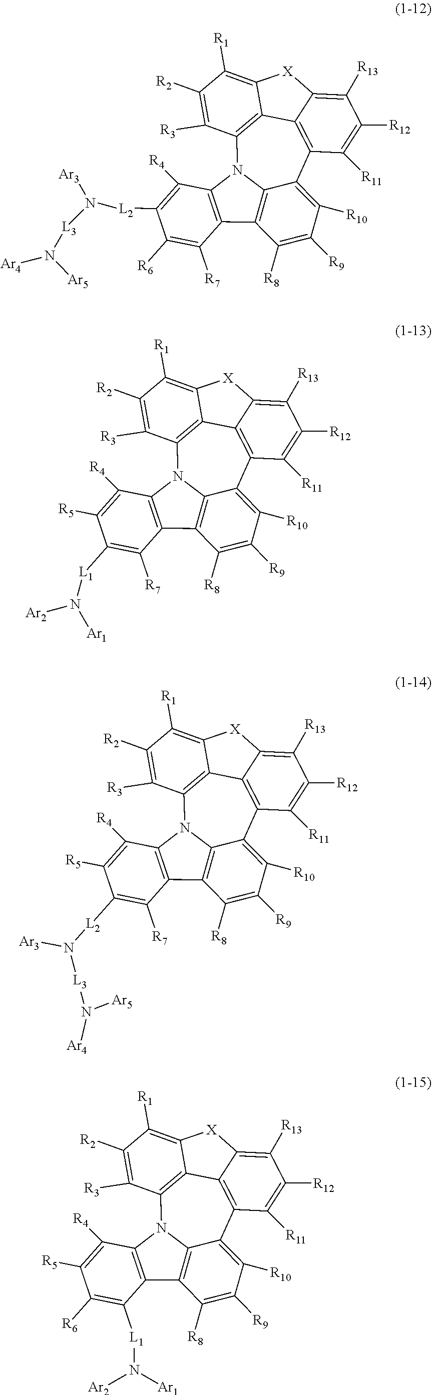 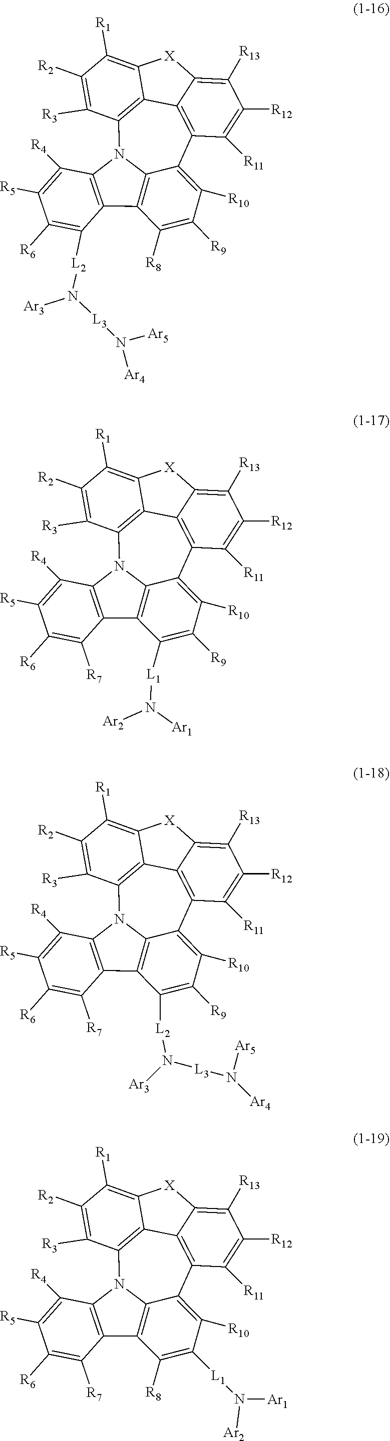 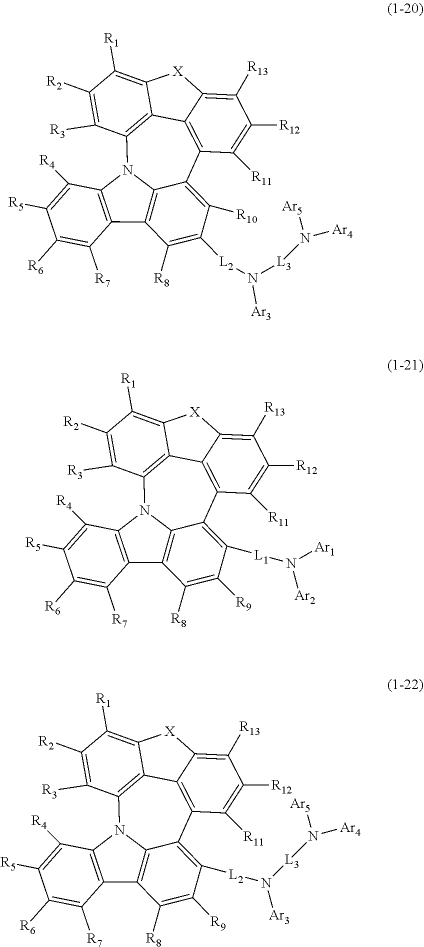 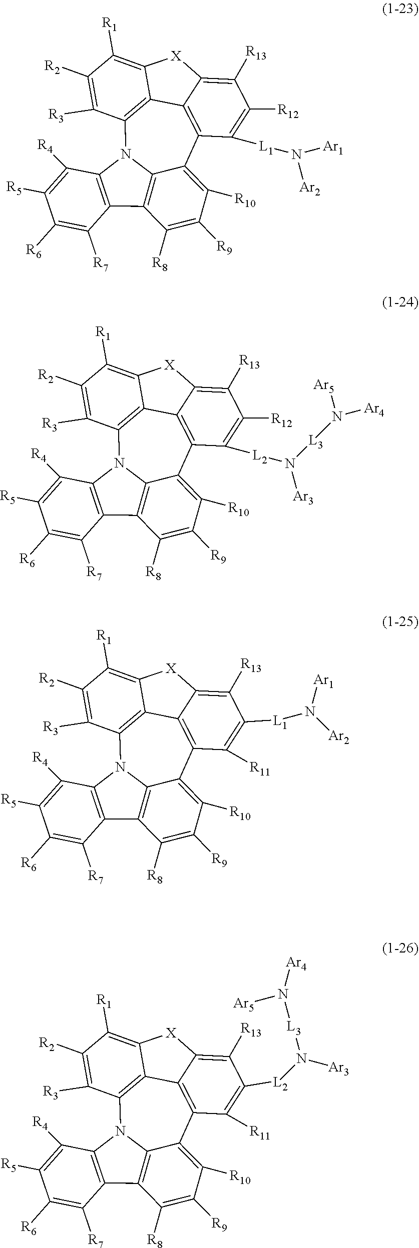 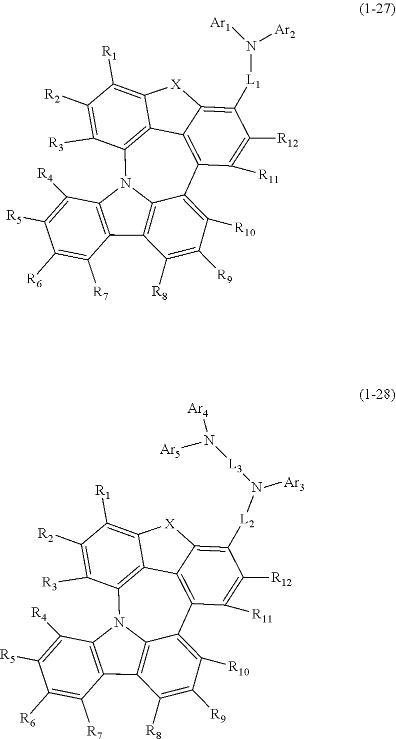
in formulas 1-1 to 1-28,
X, R₁ to R₁₃, L₁ to L₃, and Ar₁ to Ar₅ are as defined in claim 1.

4. The plurality of host materials according to claim 1, wherein Ar₁ to Ar₅ in formula 1 and Ar₆ to Ar₈ in formula 2, each independently, represent a substituted or unsubstituted phenyl, a substituted or unsubstituted naphthyl, a substituted or unsubstituted biphenyl, a substituted or unsubstituted terphenyl, a substituted or unsubstituted dibenzofuranyl, a substituted or unsubstituted dibenzothiophenyl, a substituted or unsubstituted fluorenyl, a substituted or unsubstituted spirobifluorenyl, a substituted or unsubstituted quaterphenyl, a substituted or unsubstituted cumenyl, a substituted or unsubstituted carbazolyl, a substituted or unsubstituted phenanthrenyl, a substituted or unsubstituted benzophenanthrenyl, a substituted or unsubstituted chrysenyl, a substituted or unsubstituted triphenylenyl, a substituted or unsubstituted benzocarbazolyl, a substituted or unsubstituted benzonaphthofuranyl, a substituted or unsubstituted benzonaphthothiophenyl, a substituted or unsubstituted benzofluorenyl, a substituted or unsubstituted benzothiazolyl, a substituted or unsubstituted benzoxazolyl, a substituted or unsubstituted benzimidazolyl, a substituted or unsubstituted naphthoxazolyl, a substituted or unsubstituted benzonaphthoxazolyl, a substituted or unsubstituted naphthothiazolyl, a substituted or unsubstituted benzonaphthothiazolyl, or a substituted or unsubstituted naphthoimidazolyl.

5. The plurality of host materials according to claim 1, wherein the compound represented by formula 1 is at least one selected from the group consisting of the following compounds:

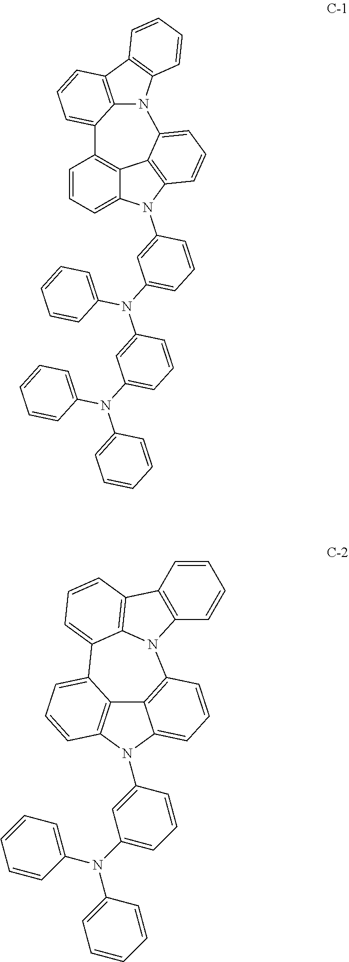 [SMILES: c1ccc(N(c2ccccc2)c2cccc(N(c3ccccc3)c3cccc(-n4c5cccc6c7cccc8c9ccccc9n(c9cccc4c9c65)c78)c3)c2)cc1 ; c1ccc(N(c2ccccc2)c2cccc(-n3c4cccc5c6cccc7c8ccccc8n(c8cccc3c8c54)c67)c2)cc1]

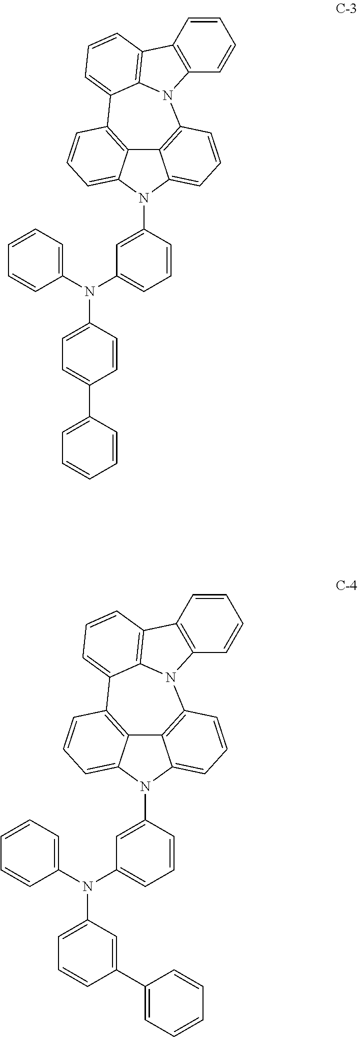 [SMILES: c1ccc(-c2ccc(N(c3ccccc3)c3cccc(-n4c5cccc6c7cccc8c9ccccc9n(c9cccc4c9c65)c78)c3)cc2)cc1 ; c1ccc(-c2cccc(N(c3ccccc3)c3cccc(-n4c5cccc6c7cccc8c9ccccc9n(c9cccc4c9c65)c78)c3)c2)cc1]

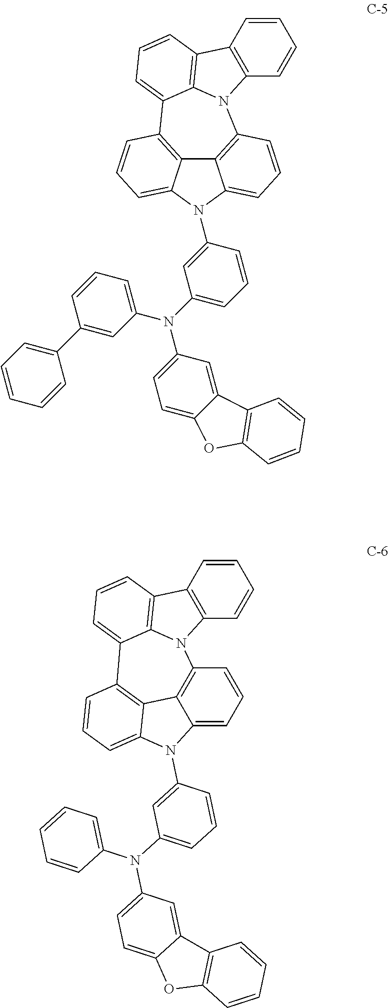 [SMILES: c1ccc(-c2cccc(N(c3cccc(-n4c5cccc6c7cccc8c9ccccc9n(c9cccc4c9c65)c78)c3)c3ccc4oc5ccccc5c4c3)c2)cc1 ; c1ccc(N(c2cccc(-n3c4cccc5c6cccc7c8ccccc8n(c8cccc3c8c54)c67)c2)c2ccc3oc4ccccc4c3c2)cc1]

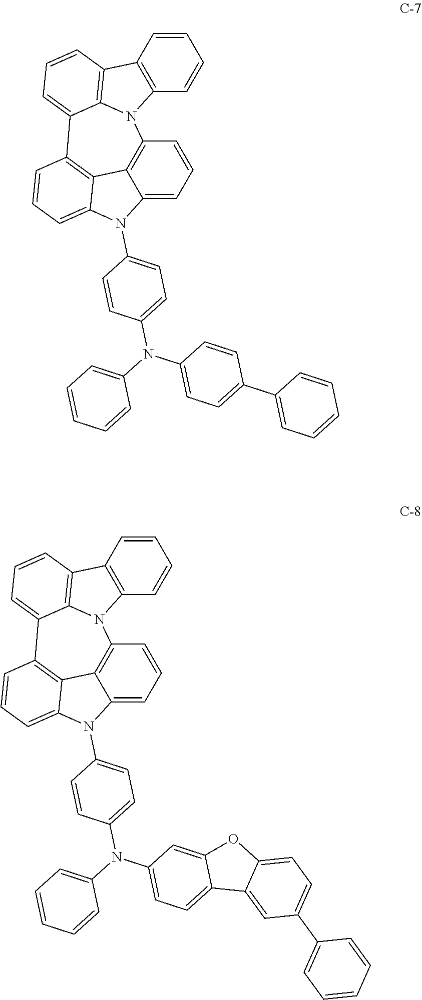 [SMILES: c1ccc(-c2ccc(N(c3ccccc3)c3ccc(-n4c5cccc6c7cccc8c9ccccc9n(c9cccc4c9c65)c78)cc3)cc2)cc1 ; c1ccc(-c2ccc3oc4cc(N(c5ccccc5)c5ccc(-n6c7cccc8c9cccc%10c%11ccccc%11n(c%11cccc6c%11c87)c9%10)cc5)ccc4c3c2)cc1]

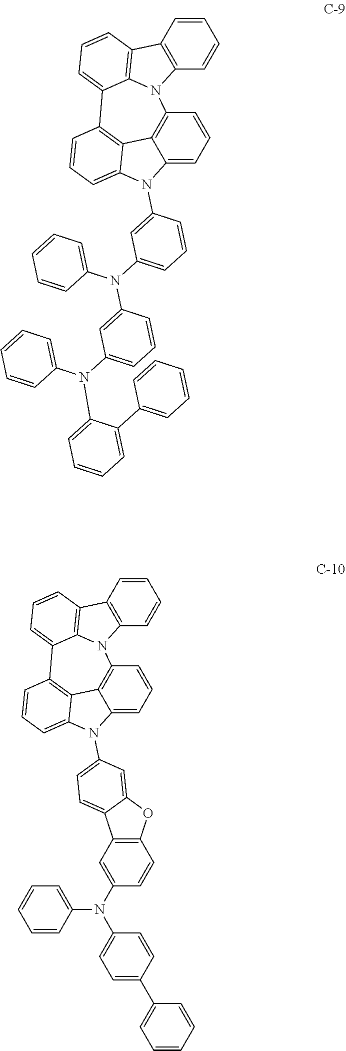 [SMILES: c1ccc(-c2ccccc2N(c2ccccc2)c2cccc(N(c3ccccc3)c3cccc(-n4c5cccc6c7cccc8c9ccccc9n(c9cccc4c9c65)c78)c3)c2)cc1 ; c1ccc(-c2ccc(N(c3ccccc3)c3ccc4oc5cc(-n6c7cccc8c9cccc%10c%11ccccc%11n(c%11cccc6c%11c87)c9%10)ccc5c4c3)cc2)cc1]

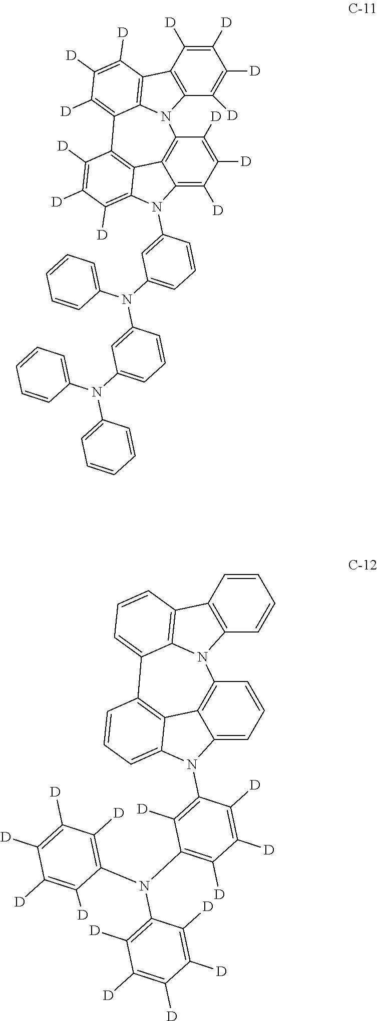 [SMILES: [2H]c1c([2H])c([2H])c2c(c1[2H])c1c([2H])c([2H])c([2H])c3c4c([2H])c([2H])c([2H])c5c4c4c(c([2H])c([2H])c([2H])c4n2c31)n5-c1cccc(N(c2ccccc2)c2cccc(N(c3ccccc3)c3ccccc3)c2)c1 ; [2H]c1c([2H])c([2H])c(N(c2c([2H])c([2H])c([2H])c([2H])c2[2H])c2c([2H])c([2H])c([2H])c(-n3c4cccc5c6cccc7c8ccccc8n(c8cccc3c8c54)c67)c2[2H])c([2H])c1[2H]]

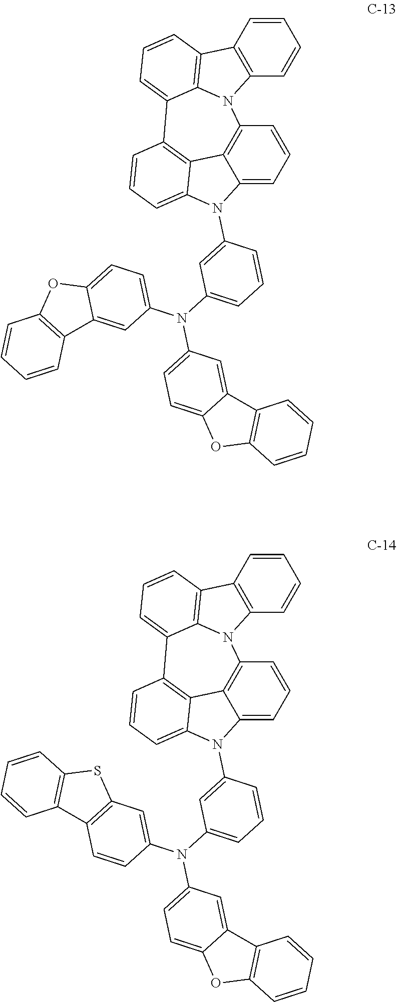 [SMILES: c1cc(N(c2ccc3oc4ccccc4c3c2)c2ccc3oc4ccccc4c3c2)cc(-n2c3cccc4c5cccc6c7ccccc7n(c7cccc2c7c43)c56)c1 ; c1cc(N(c2ccc3c(c2)sc2ccccc23)c2ccc3oc4ccccc4c3c2)cc(-n2c3cccc4c5cccc6c7ccccc7n(c7cccc2c7c43)c56)c1]

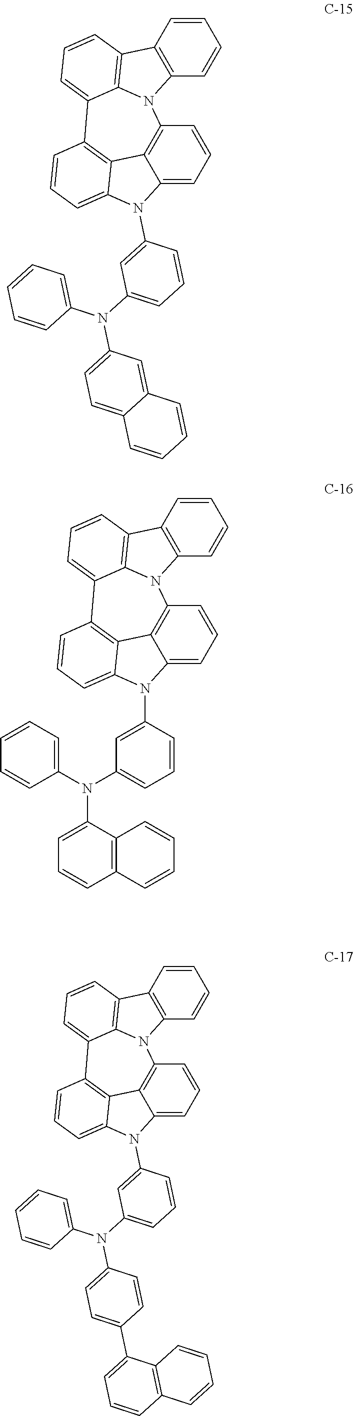

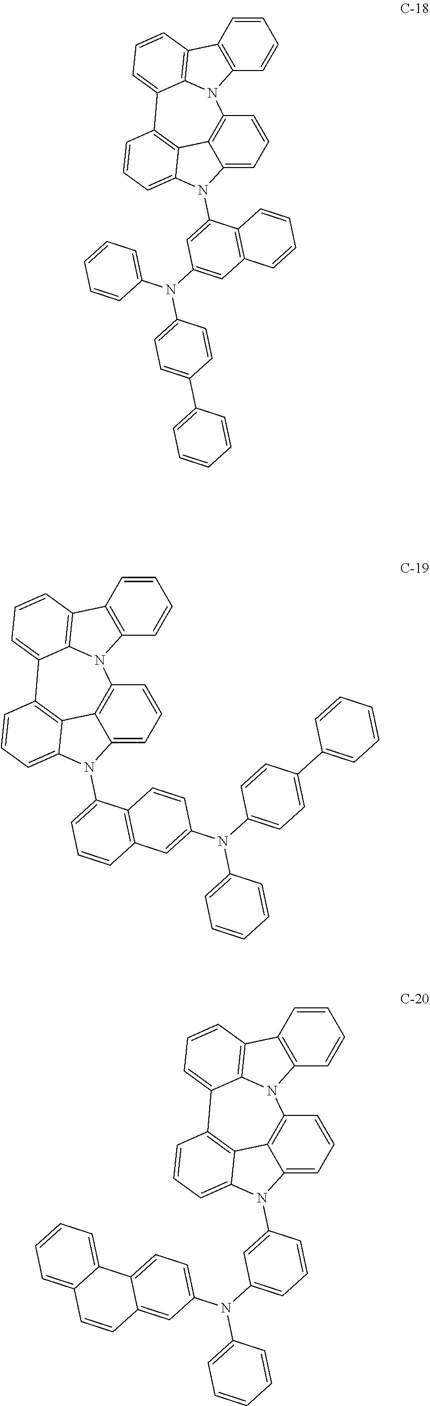 [SMILES: c1ccc(-c2ccc(N(c3ccccc3)c3cc(-n4c5cccc6c7cccc8c9ccccc9n(c9cccc4c9c65)c78)c4ccccc4c3)cc2)cc1 ; c1ccc(-c2ccc(N(c3ccccc3)c3ccc4c(-n5c6cccc7c8cccc9c%10ccccc%10n(c%10cccc5c%10c76)c89)cccc4c3)cc2)cc1 ; c1ccc(N(c2cccc(-n3c4cccc5c6cccc7c8ccccc8n(c8cccc3c8c54)c67)c2)c2ccc3c(ccc4ccccc43)c2)cc1]

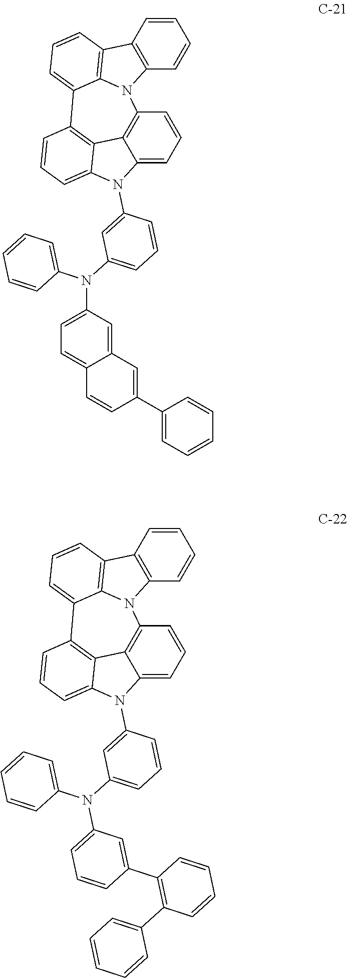 [SMILES: c1ccc(-c2ccc3ccc(N(c4ccccc4)c4cccc(-n5c6cccc7c8cccc9c%10ccccc%10n(c%10cccc5c%10c76)c89)c4)cc3c2)cc1 ; c1ccc(-c2ccccc2-c2cccc(N(c3ccccc3)c3cccc(-n4c5cccc6c7cccc8c9ccccc9n(c9cccc4c9c65)c78)c3)c2)cc1]

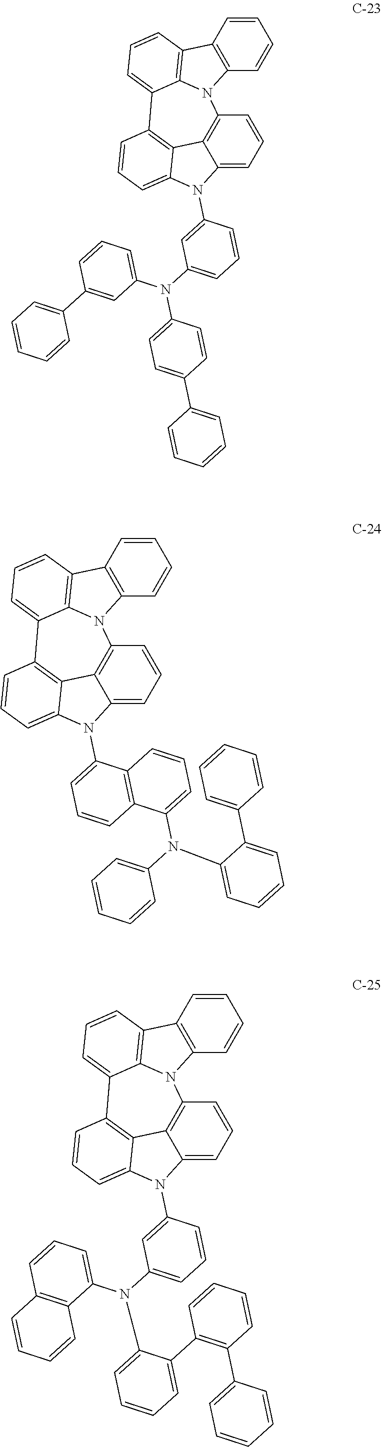 [SMILES: c1ccc(-c2ccc(N(c3cccc(-c4ccccc4)c3)c3cccc(-n4c5cccc6c7cccc8c9ccccc9n(c9cccc4c9c65)c78)c3)cc2)cc1 ; c1ccc(-c2ccccc2N(c2ccccc2)c2cccc3c(-n4c5cccc6c7cccc8c9ccccc9n(c9cccc4c9c65)c78)cccc23)cc1 ; c1ccc(-c2ccccc2-c2ccccc2N(c2cccc(-n3c4cccc5c6cccc7c8ccccc8n(c8cccc3c8c54)c67)c2)c2cccc3ccccc23)cc1]

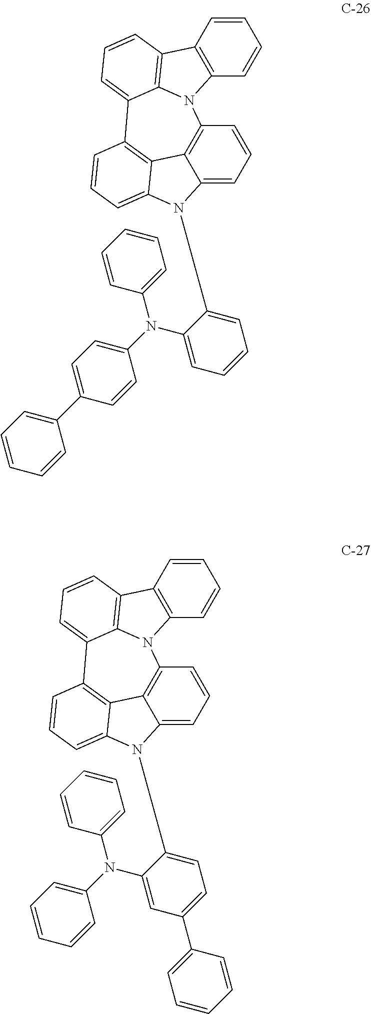 [SMILES: c1ccc(-c2ccc(N(c3ccccc3)c3ccccc3-n3c4cccc5c6cccc7c8ccccc8n(c8cccc3c8c54)c67)cc2)cc1 ; c1ccc(-c2ccc(-n3c4cccc5c6cccc7c8ccccc8n(c8cccc3c8c54)c67)c(N(c3ccccc3)c3ccccc3)c2)cc1]

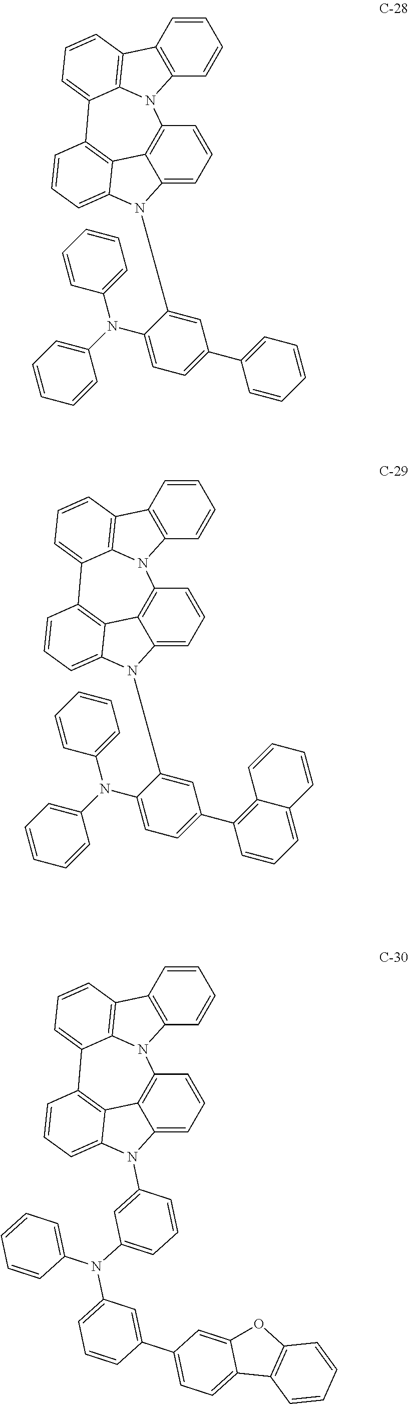 [SMILES: c1ccc(-c2ccc(N(c3ccccc3)c3ccccc3)c(-n3c4cccc5c6cccc7c8ccccc8n(c8cccc3c8c54)c67)c2)cc1 ; c1ccc(N(c2ccccc2)c2ccc(-c3cccc4ccccc34)cc2-n2c3cccc4c5cccc6c7ccccc7n(c7cccc2c7c43)c56)cc1 ; c1ccc(N(c2cccc(-c3ccc4c(c3)oc3ccccc34)c2)c2cccc(-n3c4cccc5c6cccc7c8ccccc8n(c8cccc3c8c54)c67)c2)cc1]

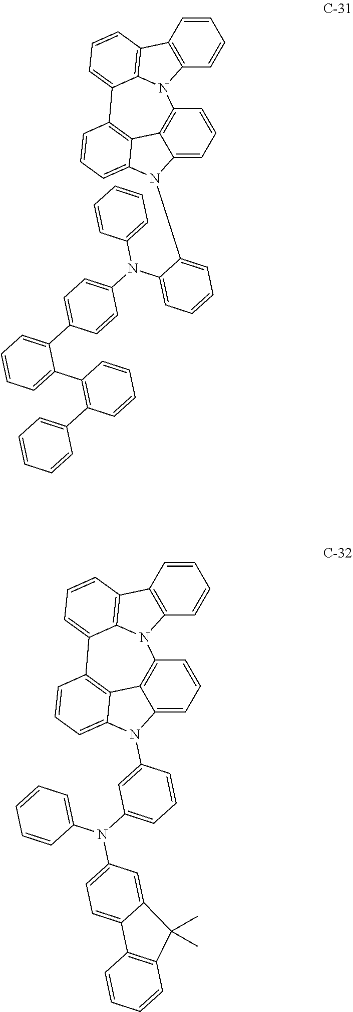 [SMILES: c1ccc(-c2ccccc2-c2ccccc2-c2ccc(N(c3ccccc3)c3ccccc3-n3c4cccc5c6cccc7c8ccccc8n(c8cccc3c8c54)c67)cc2)cc1 ; CC1(C)c2ccccc2-c2ccc(N(c3ccccc3)c3cccc(-n4c5cccc6c7cccc8c9ccccc9n(c9cccc4c9c65)c78)c3)cc21]

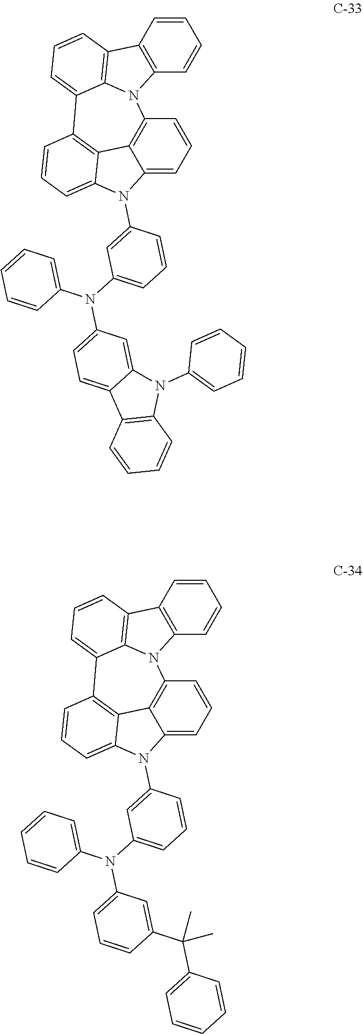 [SMILES: c1ccc(N(c2cccc(-n3c4cccc5c6cccc7c8ccccc8n(c8cccc3c8c54)c67)c2)c2ccc3c4ccccc4n(-c4ccccc4)c3c2)cc1 ; CC(C)(c1ccccc1)c1cccc(N(c2ccccc2)c2cccc(-n3c4cccc5c6cccc7c8ccccc8n(c8cccc3c8c54)c67)c2)c1]

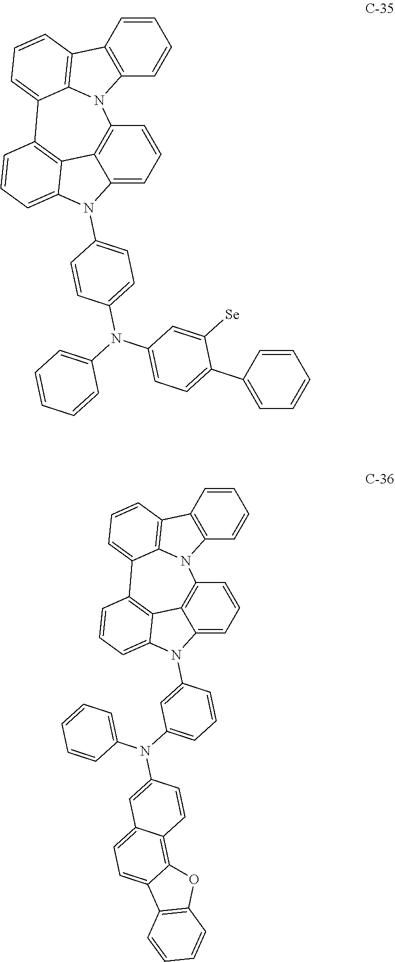

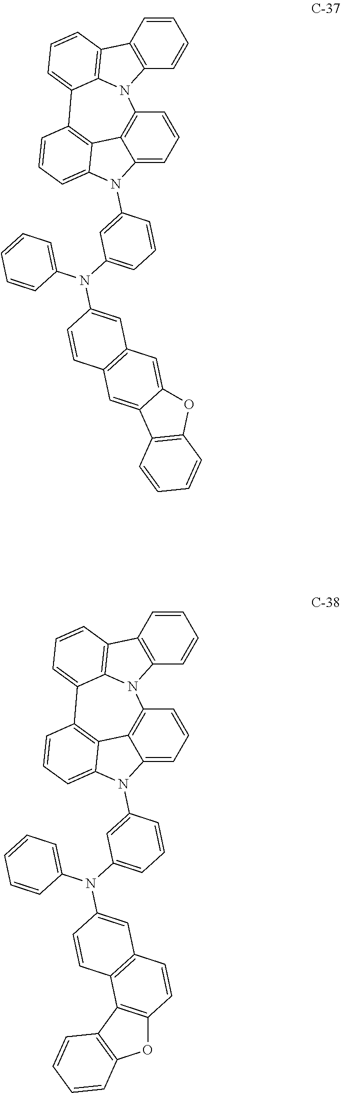 [SMILES: c1ccc(N(c2cccc(-n3c4cccc5c6cccc7c8ccccc8n(c8cccc3c8c54)c67)c2)c2ccc3cc4c(cc3c2)oc2ccccc24)cc1 ; c1ccc(N(c2cccc(-n3c4cccc5c6cccc7c8ccccc8n(c8cccc3c8c54)c67)c2)c2ccc3c(ccc4oc5ccccc5c43)c2)cc1]

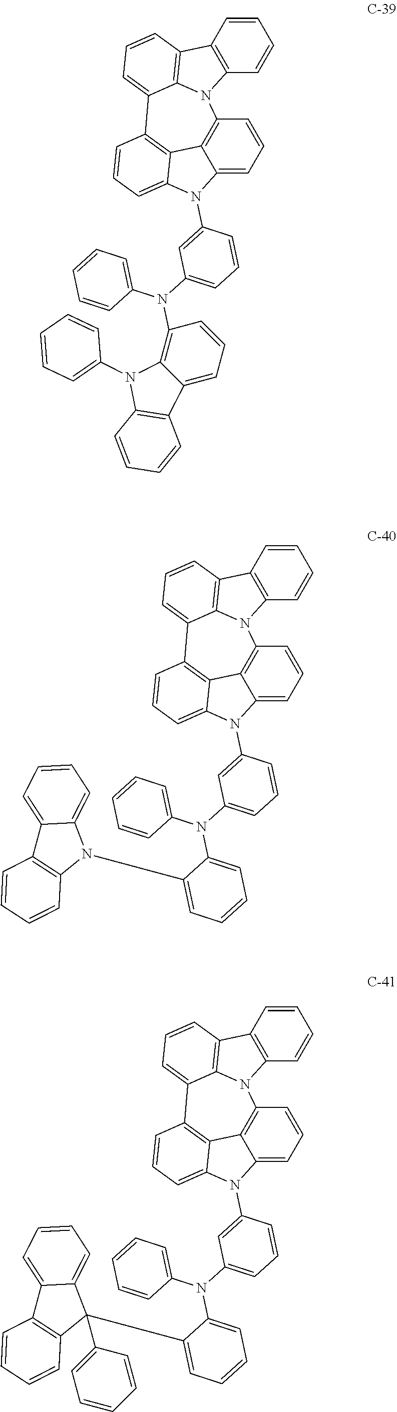 [SMILES: c1ccc(N(c2cccc(-n3c4cccc5c6cccc7c8ccccc8n(c8cccc3c8c54)c67)c2)c2cccc3c4ccccc4n(-c4ccccc4)c23)cc1 ; c1ccc(N(c2cccc(-n3c4cccc5c6cccc7c8ccccc8n(c8cccc3c8c54)c67)c2)c2ccccc2-n2c3ccccc3c3ccccc32)cc1 ; c1ccc(N(c2cccc(-n3c4cccc5c6cccc7c8ccccc8n(c8cccc3c8c54)c67)c2)c2ccccc2C2(c3ccccc3)c3ccccc3-c3ccccc32)cc1]

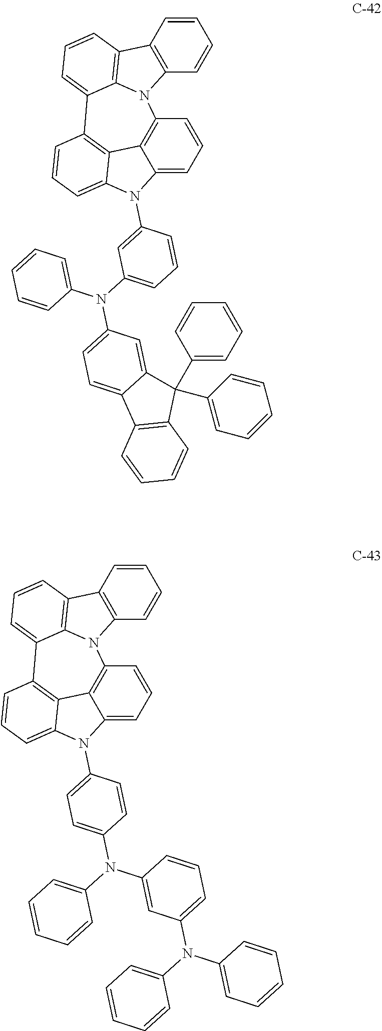 [SMILES: c1ccc(N(c2cccc(-n3c4cccc5c6cccc7c8ccccc8n(c8cccc3c8c54)c67)c2)c2ccc3c(c2)C(c2ccccc2)(c2ccccc2)c2ccccc2-3)cc1 ; c1ccc(N(c2ccccc2)c2cccc(N(c3ccccc3)c3ccc(-n4c5cccc6c7cccc8c9ccccc9n(c9cccc4c9c65)c78)cc3)c2)cc1]

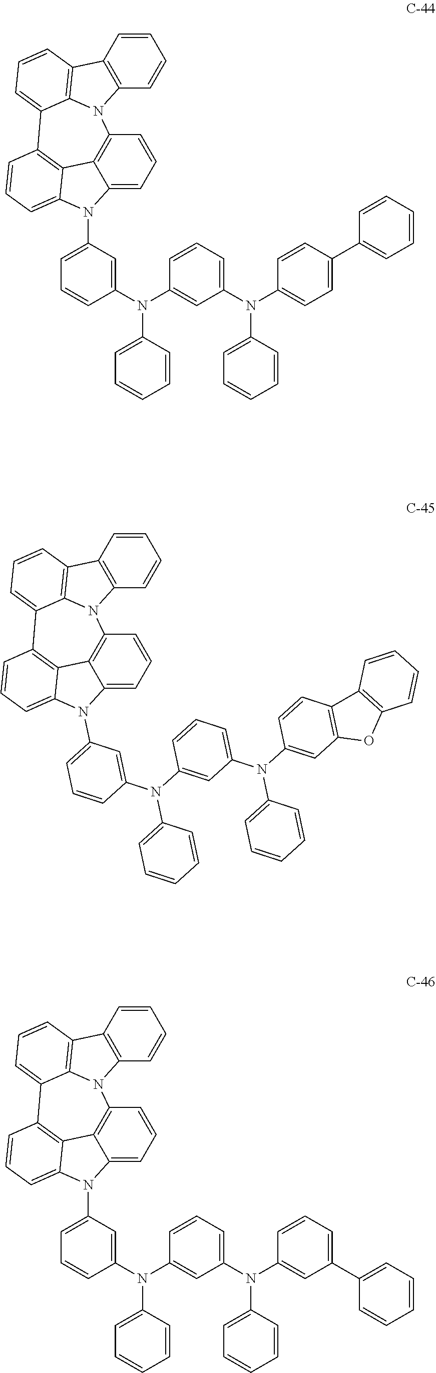 [SMILES: c1ccc(-c2ccc(N(c3ccccc3)c3cccc(N(c4ccccc4)c4cccc(-n5c6cccc7c8cccc9c%10ccccc%10n(c%10cccc5c%10c76)c89)c4)c3)cc2)cc1 ; c1ccc(N(c2cccc(N(c3ccccc3)c3ccc4c(c3)oc3ccccc34)c2)c2cccc(-n3c4cccc5c6cccc7c8ccccc8n(c8cccc3c8c54)c67)c2)cc1 ; c1ccc(-c2cccc(N(c3ccccc3)c3cccc(N(c4ccccc4)c4cccc(-n5c6cccc7c8cccc9c%10ccccc%10n(c%10cccc5c%10c76)c89)c4)c3)c2)cc1]

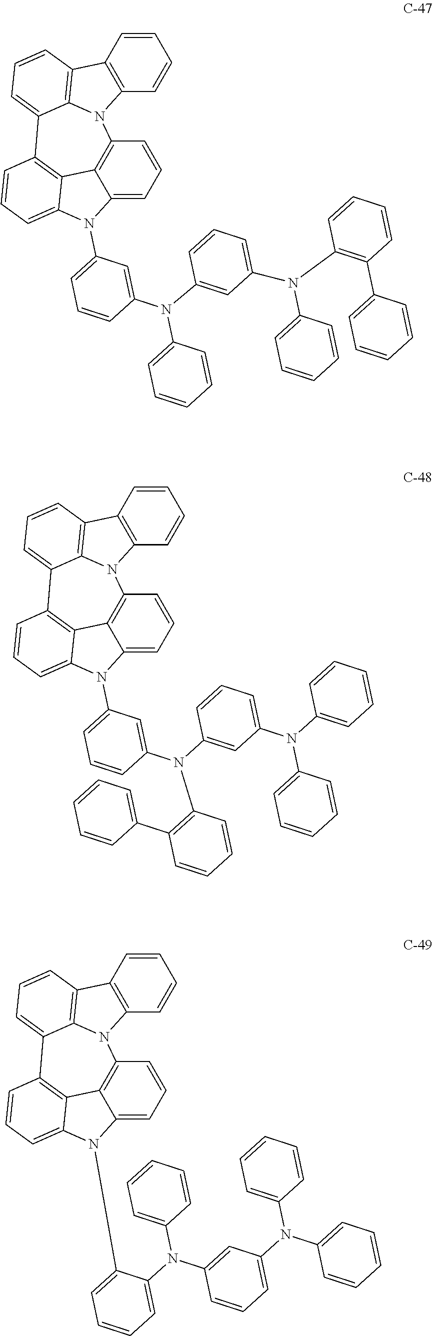 [SMILES: c1ccc(-c2ccccc2N(c2ccccc2)c2cccc(N(c3ccccc3)c3cccc(-n4c5cccc6c7cccc8c9ccccc9n(c9cccc4c9c65)c78)c3)c2)cc1 ; c1ccc(-c2ccccc2N(c2cccc(N(c3ccccc3)c3ccccc3)c2)c2cccc(-n3c4cccc5c6cccc7c8ccccc8n(c8cccc3c8c54)c67)c2)cc1 ; c1ccc(N(c2ccccc2)c2cccc(N(c3ccccc3)c3ccccc3-n3c4cccc5c6cccc7c8ccccc8n(c8cccc3c8c54)c67)c2)cc1]

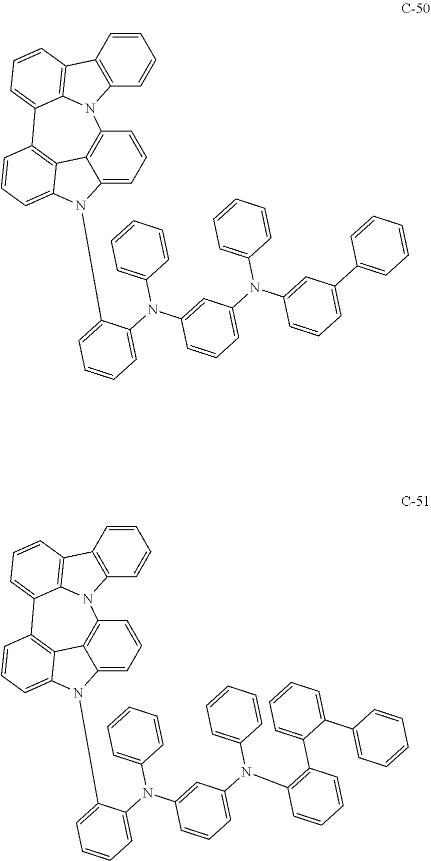 [SMILES: c1ccc(-c2cccc(N(c3ccccc3)c3cccc(N(c4ccccc4)c4ccccc4-n4c5cccc6c7cccc8c9ccccc9n(c9cccc4c9c65)c78)c3)c2)cc1 ; c1ccc(-c2ccccc2-c2ccccc2N(c2ccccc2)c2cccc(N(c3ccccc3)c3ccccc3-n3c4cccc5c6cccc7c8ccccc8n(c8cccc3c8c54)c67)c2)cc1]

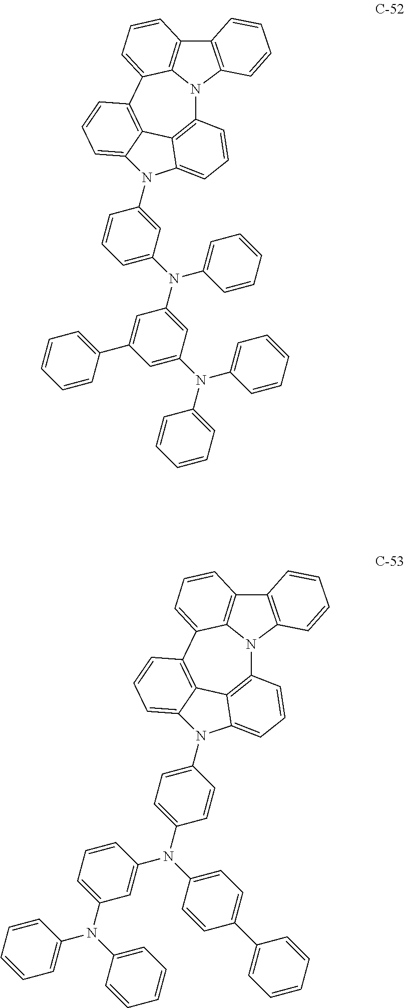 [SMILES: c1ccc(-c2cc(N(c3ccccc3)c3ccccc3)cc(N(c3ccccc3)c3cccc(-n4c5cccc6c7cccc8c9ccccc9n(c9cccc4c9c65)c78)c3)c2)cc1 ; c1ccc(-c2ccc(N(c3ccc(-n4c5cccc6c7cccc8c9ccccc9n(c9cccc4c9c65)c78)cc3)c3cccc(N(c4ccccc4)c4ccccc4)c3)cc2)cc1]

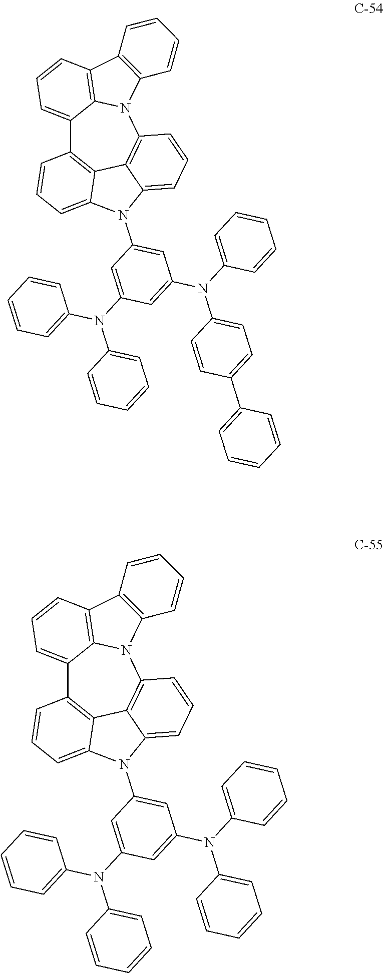 [SMILES: c1ccc(-c2ccc(N(c3ccccc3)c3cc(N(c4ccccc4)c4ccccc4)cc(-n4c5cccc6c7cccc8c9ccccc9n(c9cccc4c9c65)c78)c3)cc2)cc1 ; c1ccc(N(c2ccccc2)c2cc(N(c3ccccc3)c3ccccc3)cc(-n3c4cccc5c6cccc7c8ccccc8n(c8cccc3c8c54)c67)c2)cc1]

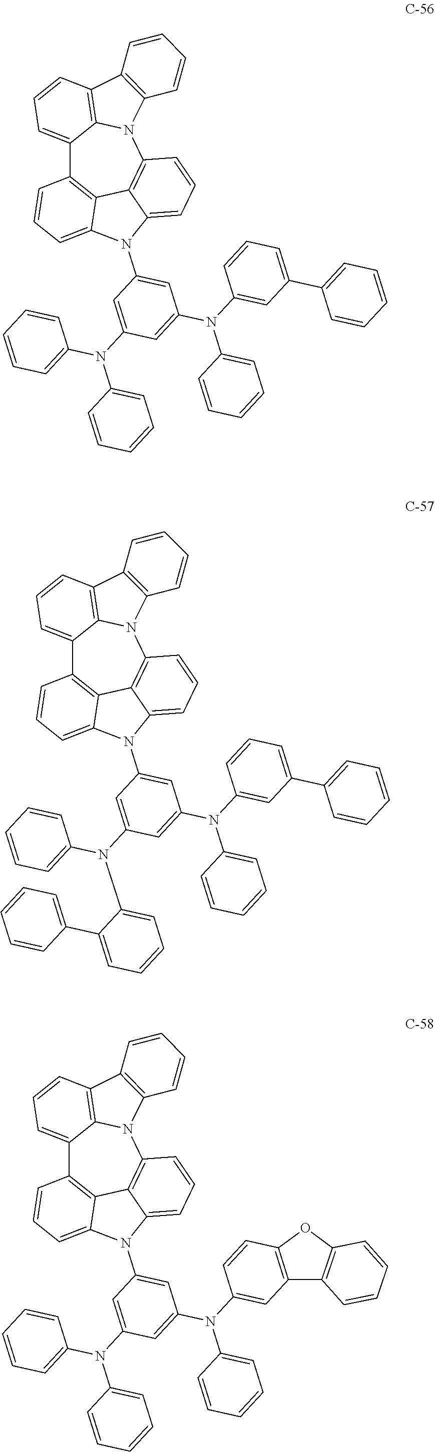 [SMILES: c1ccc(-c2cccc(N(c3ccccc3)c3cc(N(c4ccccc4)c4ccccc4)cc(-n4c5cccc6c7cccc8c9ccccc9n(c9cccc4c9c65)c78)c3)c2)cc1 ; c1ccc(-c2cccc(N(c3ccccc3)c3cc(N(c4ccccc4)c4ccccc4-c4ccccc4)cc(-n4c5cccc6c7cccc8c9ccccc9n(c9cccc4c9c65)c78)c3)c2)cc1 ; c1ccc(N(c2ccccc2)c2cc(N(c3ccccc3)c3ccc4oc5ccccc5c4c3)cc(-n3c4cccc5c6cccc7c8ccccc8n(c8cccc3c8c54)c67)c2)cc1]

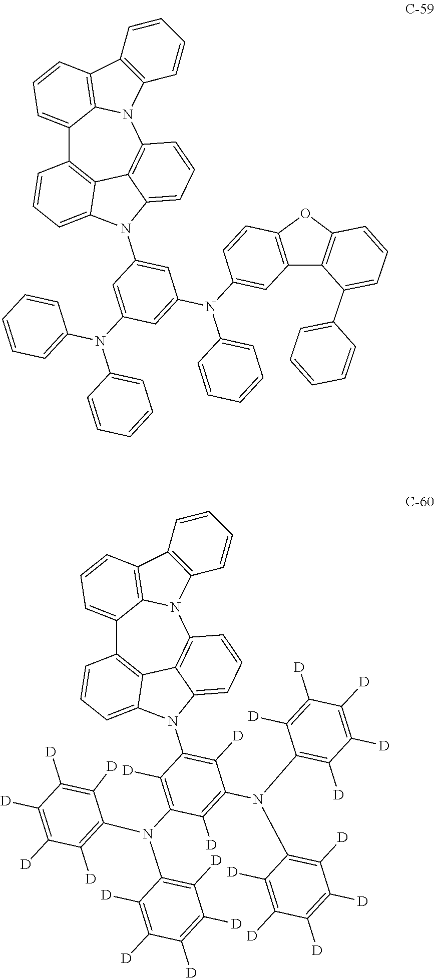 [SMILES: c1ccc(-c2cccc3oc4ccc(N(c5ccccc5)c5cc(N(c6ccccc6)c6ccccc6)cc(-n6c7cccc8c9cccc%10c%11ccccc%11n(c%11cccc6c%11c87)c9%10)c5)cc4c23)cc1 ; [2H]c1c([2H])c([2H])c(N(c2c([2H])c([2H])c([2H])c([2H])c2[2H])c2c([2H])c(N(c3c([2H])c([2H])c([2H])c([2H])c3[2H])c3c([2H])c([2H])c([2H])c([2H])c3[2H])c([2H])c(-n3c4cccc5c6cccc7c8ccccc8n(c8cccc3c8c54)c67)c2[2H])c([2H])c1[2H]]

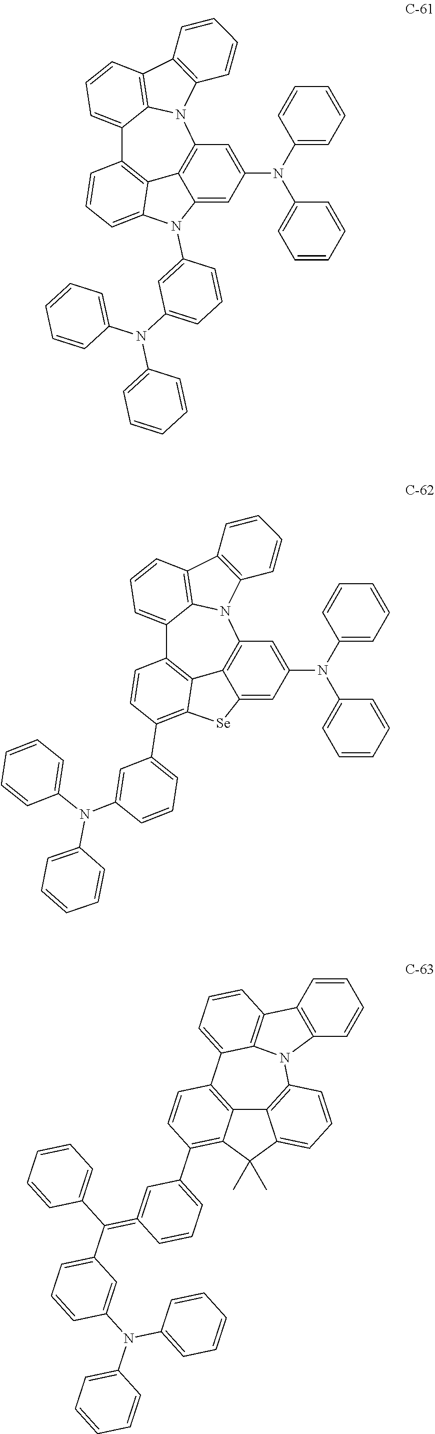

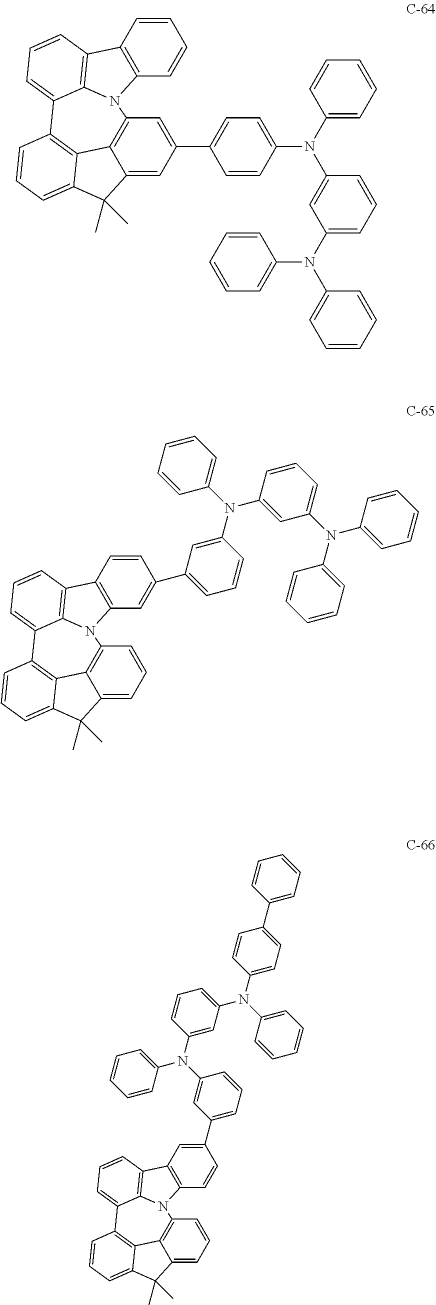 [SMILES: CC1(C)c2cccc3c2-c2c(cc(-c4ccc(N(c5ccccc5)c5cccc(N(c6ccccc6)c6ccccc6)c5)cc4)cc21)-n1c2ccccc2c2cccc-3c21 ; CC1(C)c2cccc3c2-c2c(cccc21)-n1c2cc(-c4cccc(N(c5ccccc5)c5cccc(N(c6ccccc6)c6ccccc6)c5)c4)ccc2c2cccc-3c21 ; CC1(C)c2cccc3c2-c2c(cccc21)-n1c2ccc(-c4cccc(N(c5ccccc5)c5cccc(N(c6ccccc6)c6ccc(-c7ccccc7)cc6)c5)c4)cc2c2cccc-3c21]

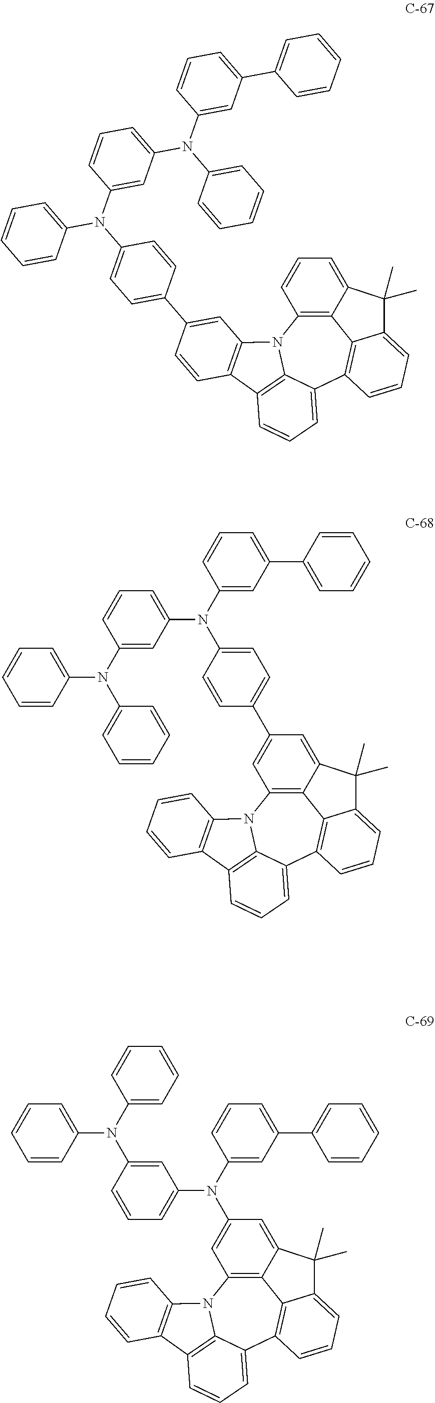 [SMILES: CC1(C)c2cccc3c2-c2c(cccc21)-n1c2cc(-c4ccc(N(c5ccccc5)c5cccc(N(c6ccccc6)c6cccc(-c7ccccc7)c6)c5)cc4)ccc2c2cccc-3c21 ; CC1(C)c2cccc3c2-c2c(cc(-c4ccc(N(c5cccc(-c6ccccc6)c5)c5cccc(N(c6ccccc6)c6ccccc6)c5)cc4)cc21)-n1c2ccccc2c2cccc-3c21 ; CC1(C)c2cccc3c2-c2c(cc(N(c4cccc(-c5ccccc5)c4)c4cccc(N(c5ccccc5)c5ccccc5)c4)cc21)-n1c2ccccc2c2cccc-3c21]

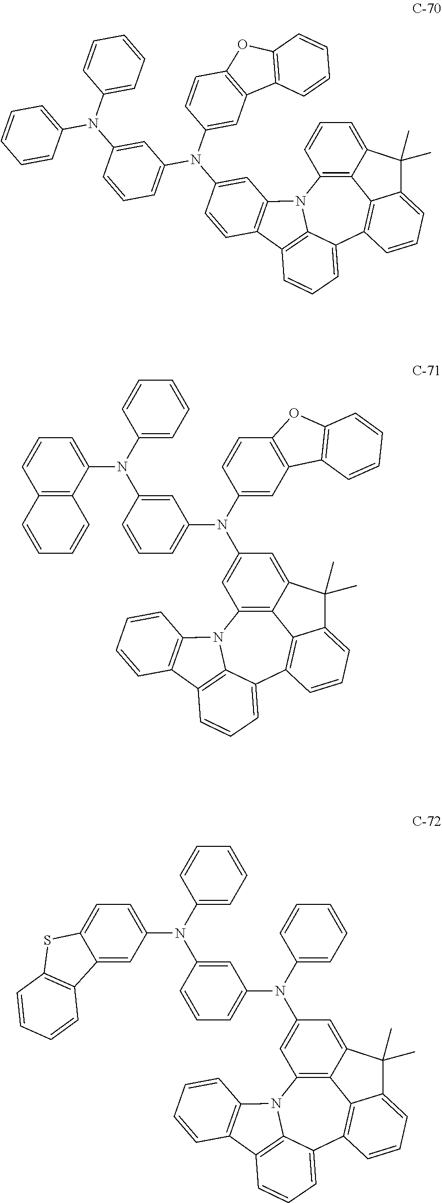 [SMILES: CC1(C)c2cccc3c2-c2c(cccc21)-n1c2cc(N(c4cccc(N(c5ccccc5)c5ccccc5)c4)c4ccc5oc6ccccc6c5c4)ccc2c2cccc-3c21 ; CC1(C)c2cccc3c2-c2c(cc(N(c4cccc(N(c5ccccc5)c5cccc6ccccc56)c4)c4ccc5oc6ccccc6c5c4)cc21)-n1c2ccccc2c2cccc-3c21 ; CC1(C)c2cccc3c2-c2c(cc(N(c4ccccc4)c4cccc(N(c5ccccc5)c5ccc6sc7ccccc7c6c5)c4)cc21)-n1c2ccccc2c2cccc-3c21]

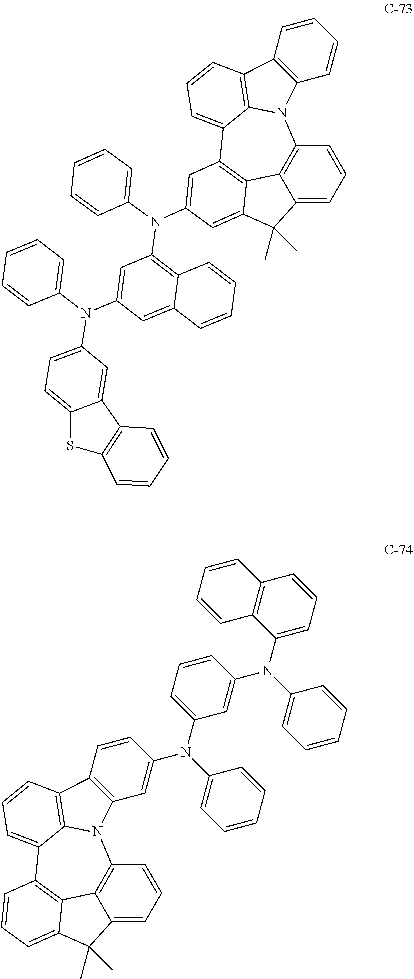 [SMILES: CC1(C)c2cc(N(c3ccccc3)c3cc(N(c4ccccc4)c4ccc5sc6ccccc6c5c4)cc4ccccc34)cc3c2-c2c(cccc21)-n1c2ccccc2c2cccc-3c21 ; CC1(C)c2cccc3c2-c2c(cccc21)-n1c2cc(N(c4ccccc4)c4cccc(N(c5ccccc5)c5cccc6ccccc56)c4)ccc2c2cccc-3c21]

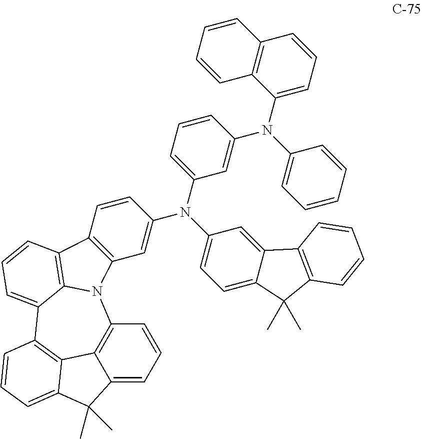 [SMILES: CC1(C)c2ccccc2-c2cc(N(c3cccc(N(c4ccccc4)c4cccc5ccccc45)c3)c3ccc4c5cccc6c5n(c4c3)-c3cccc4c3-c3c-6cccc3C4(C)C)ccc21]

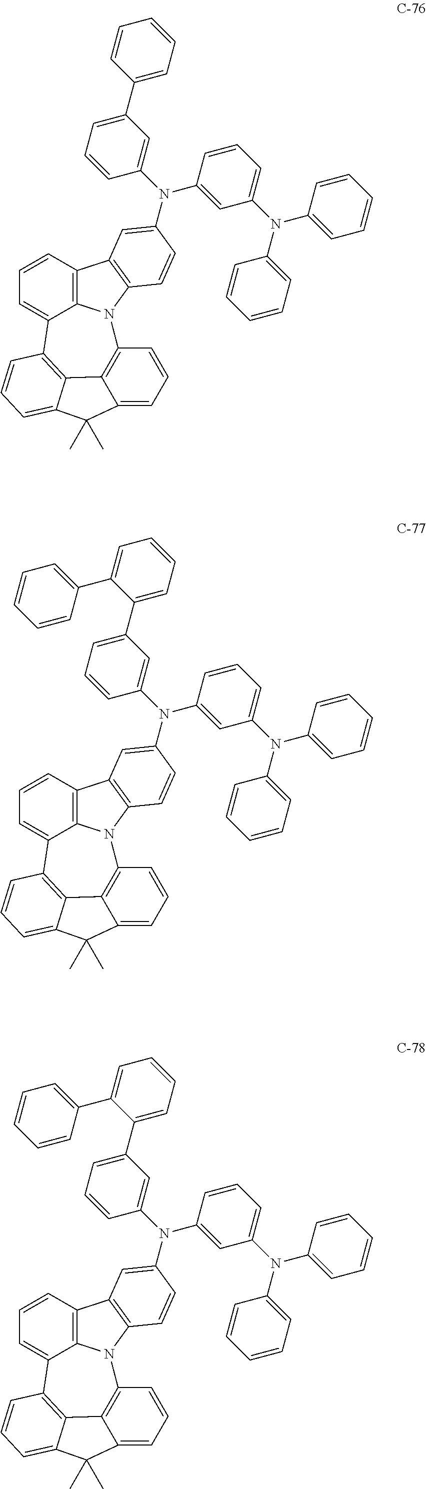 [SMILES: CC1(C)c2cccc3c2-c2c(cccc21)-n1c2ccc(N(c4cccc(-c5ccccc5)c4)c4cccc(N(c5ccccc5)c5ccccc5)c4)cc2c2cccc-3c21 ; CC1(C)c2cccc3c2-c2c(cccc21)-n1c2ccc(N(c4cccc(-c5ccccc5-c5ccccc5)c4)c4cccc(N(c5ccccc5)c5ccccc5)c4)cc2c2cccc-3c21 ; CC1(C)c2cccc3c2-c2c(cccc21)-n1c2ccc(N(c4cccc(-c5ccccc5-c5ccccc5)c4)c4cccc(N(c5ccccc5)c5ccccc5)c4)cc2c2cccc-3c21]

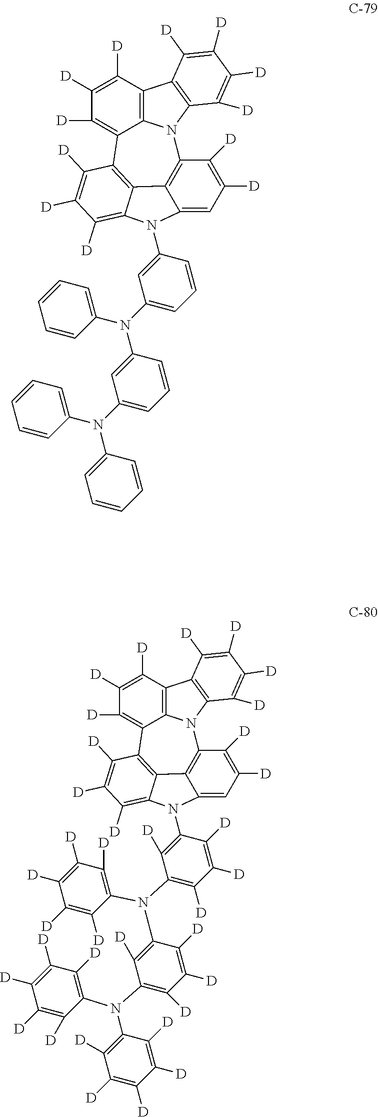 [SMILES: [2H]c1cc2c3c4c(c([2H])c([2H])c([2H])c4n2-c2cccc(N(c4ccccc4)c4cccc(N(c5ccccc5)c5ccccc5)c4)c2)c2c([2H])c([2H])c([2H])c4c5c([2H])c([2H])c([2H])c([2H])c5n(c3c1[2H])c24 ; [2H]c1cc2c3c4c(c([2H])c([2H])c([2H])c4n2-c2c([2H])c([2H])c([2H])c(N(c4c([2H])c([2H])c([2H])c([2H])c4[2H])c4c([2H])c([2H])c([2H])c(N(c5c([2H])c([2H])c([2H])c([2H])c5[2H])c5c([2H])c([2H])c([2H])c([2H])c5[2H])c4[2H])c2[2H])c2c([2H])c([2H])c([2H])c4c5c([2H])c([2H])c([2H])c([2H])c5n(c3c1[2H])c24]

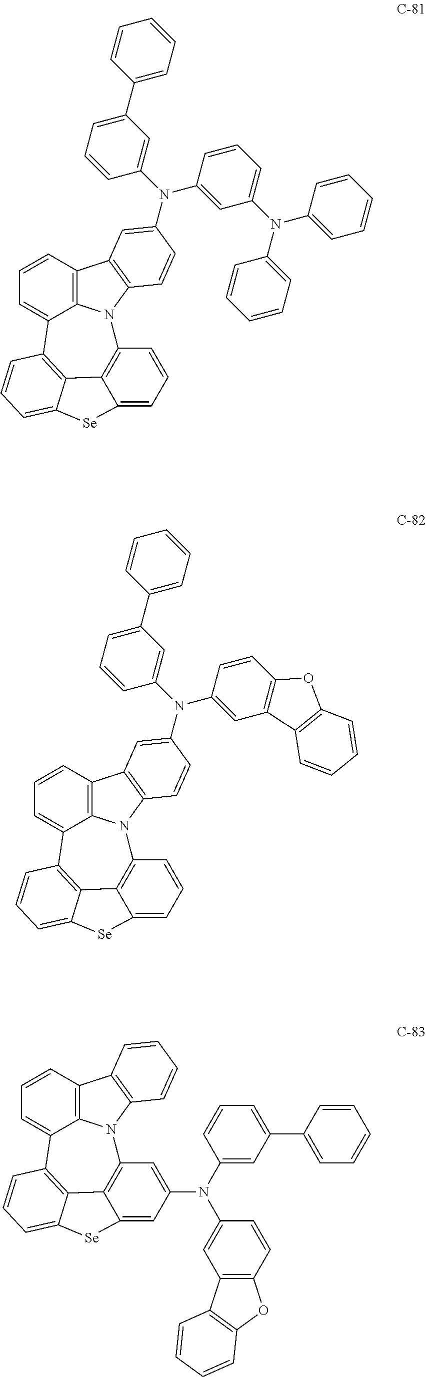 [SMILES: c1ccc(-c2cccc(N(c3cccc(N(c4ccccc4)c4ccccc4)c3)c3ccc4c(c3)c3cccc5c6cccc7[se]c8cccc(c8c76)n4c53)c2)cc1 ; c1ccc(-c2cccc(N(c3ccc4oc5ccccc5c4c3)c3ccc4c(c3)c3cccc5c6cccc7[se]c8cccc(c8c76)n4c53)c2)cc1 ; c1ccc(-c2cccc(N(c3ccc4oc5ccccc5c4c3)c3cc4[se]c5cccc6c7cccc8c9ccccc9n(c(c3)c4c56)c78)c2)cc1]

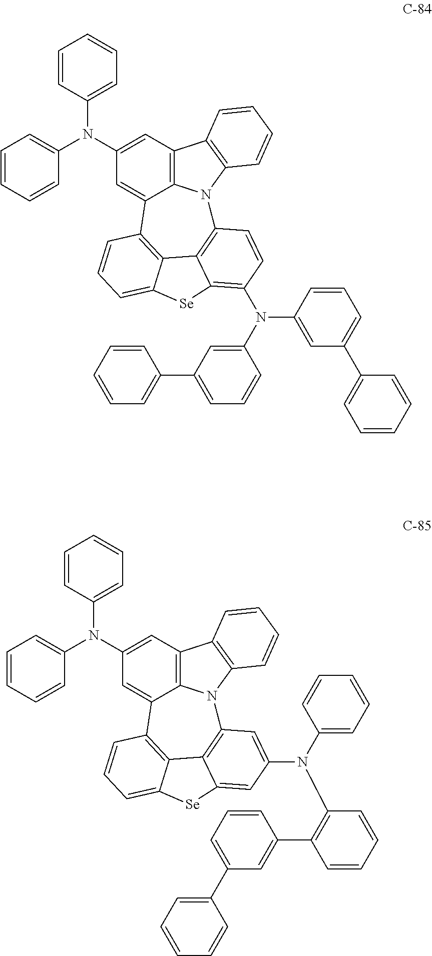 [SMILES: c1ccc(-c2cccc(N(c3cccc(-c4ccccc4)c3)c3ccc4c5c3[se]c3cccc(c6cc(N(c7ccccc7)c7ccccc7)cc7c8ccccc8n4c67)c35)c2)cc1 ; c1ccc(-c2cccc(-c3ccccc3N(c3ccccc3)c3cc4[se]c5cccc6c7cc(N(c8ccccc8)c8ccccc8)cc8c9ccccc9n(c(c3)c4c56)c78)c2)cc1]

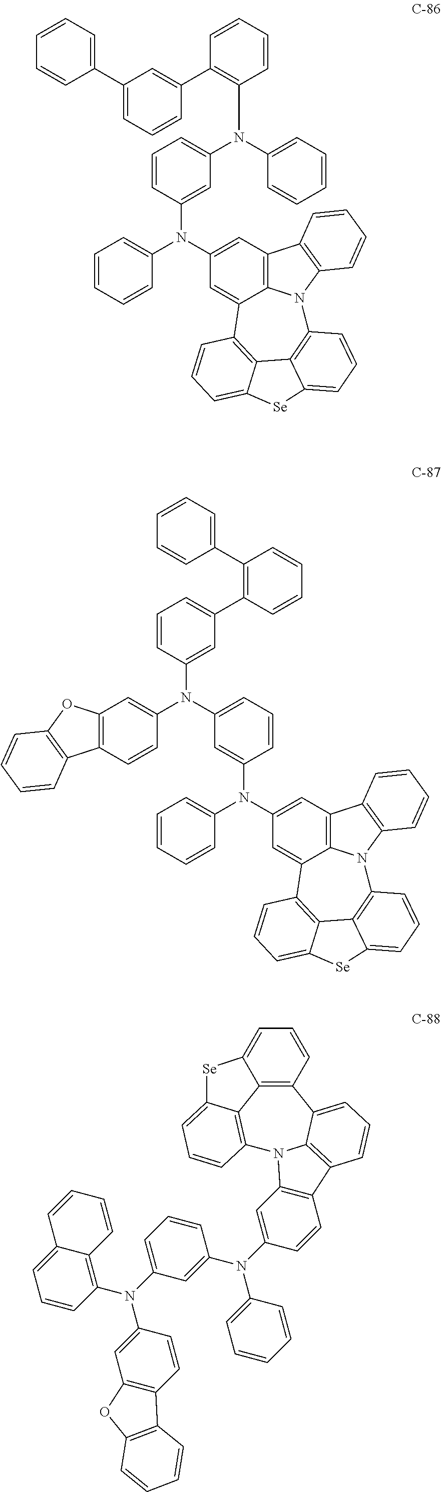 [SMILES: c1ccc(-c2cccc(-c3ccccc3N(c3ccccc3)c3cccc(N(c4ccccc4)c4cc5c6cccc7[se]c8cccc(c8c76)n6c7ccccc7c(c4)c56)c3)c2)cc1 ; c1ccc(-c2ccccc2-c2cccc(N(c3cccc(N(c4ccccc4)c4cc5c6cccc7[se]c8cccc(c8c76)n6c7ccccc7c(c4)c56)c3)c3ccc4c(c3)oc3ccccc34)c2)cc1 ; c1ccc(N(c2cccc(N(c3ccc4c(c3)oc3ccccc34)c3cccc4ccccc34)c2)c2ccc3c4cccc5c6cccc7[se]c8cccc(c8c76)n(c3c2)c54)cc1]

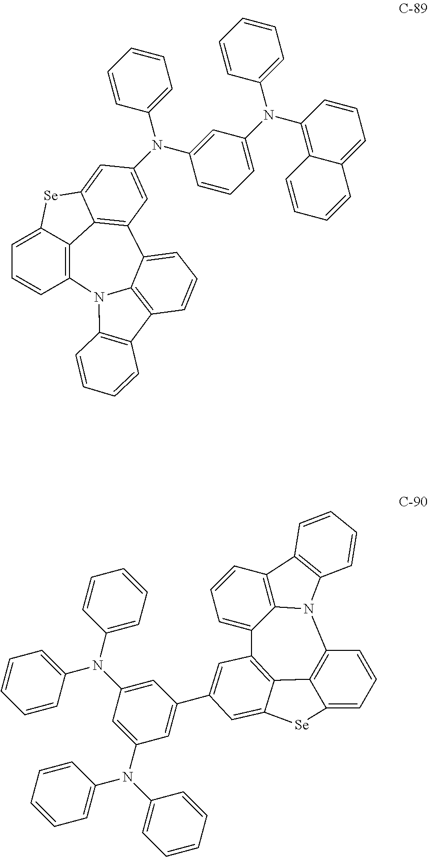 [SMILES: c1ccc(N(c2cccc(N(c3ccccc3)c3cccc4ccccc34)c2)c2cc3[se]c4cccc5c4c3c(c2)c2cccc3c4ccccc4n5c23)cc1 ; c1ccc(N(c2ccccc2)c2cc(-c3cc4[se]c5cccc6c5c4c(c3)c3cccc4c5ccccc5n6c34)cc(N(c3ccccc3)c3ccccc3)c2)cc1]

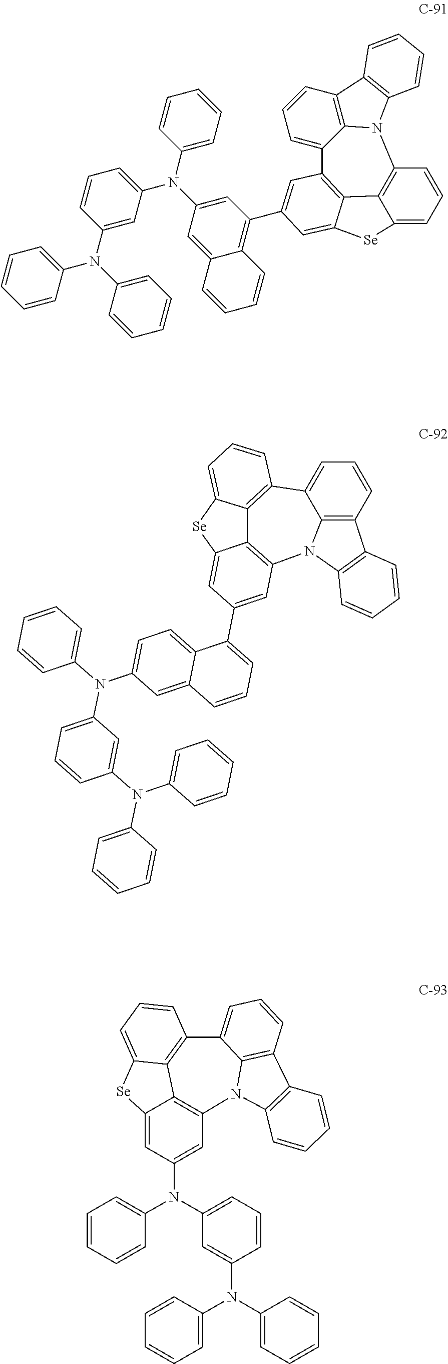 [SMILES: c1ccc(N(c2ccccc2)c2cccc(N(c3ccccc3)c3cc(-c4cc5[se]c6cccc7c6c5c(c4)c4cccc5c6ccccc6n7c45)c4ccccc4c3)c2)cc1 ; c1ccc(N(c2ccccc2)c2cccc(N(c3ccccc3)c3ccc4c(-c5cc6[se]c7cccc8c9cccc%10c%11ccccc%11n(c(c5)c6c78)c9%10)cccc4c3)c2)cc1 ; c1ccc(N(c2ccccc2)c2cccc(N(c3ccccc3)c3cc4[se]c5cccc6c7cccc8c9ccccc9n(c(c3)c4c56)c78)c2)cc1]

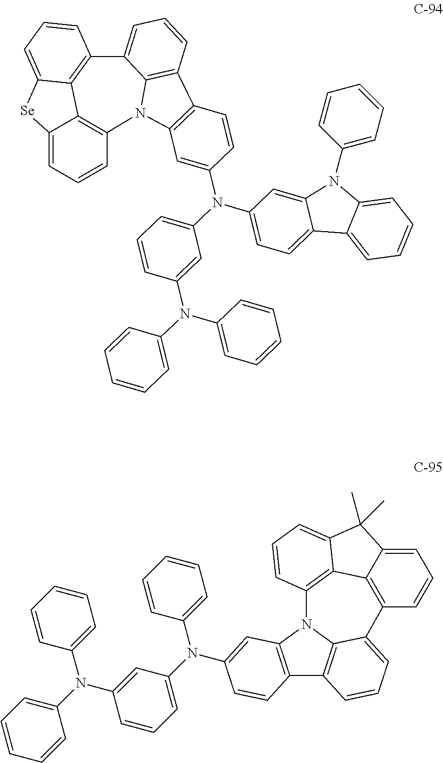 [SMILES: c1ccc(N(c2ccccc2)c2cccc(N(c3ccc4c5ccccc5n(-c5ccccc5)c4c3)c3ccc4c5cccc6c7cccc8[se]c9cccc(c9c87)n(c4c3)c65)c2)cc1 ; CC1(C)c2cccc3c2-c2c(cccc21)-n1c2cc(N(c4ccccc4)c4cccc(N(c5ccccc5)c5ccccc5)c4)ccc2c2cccc-3c21]

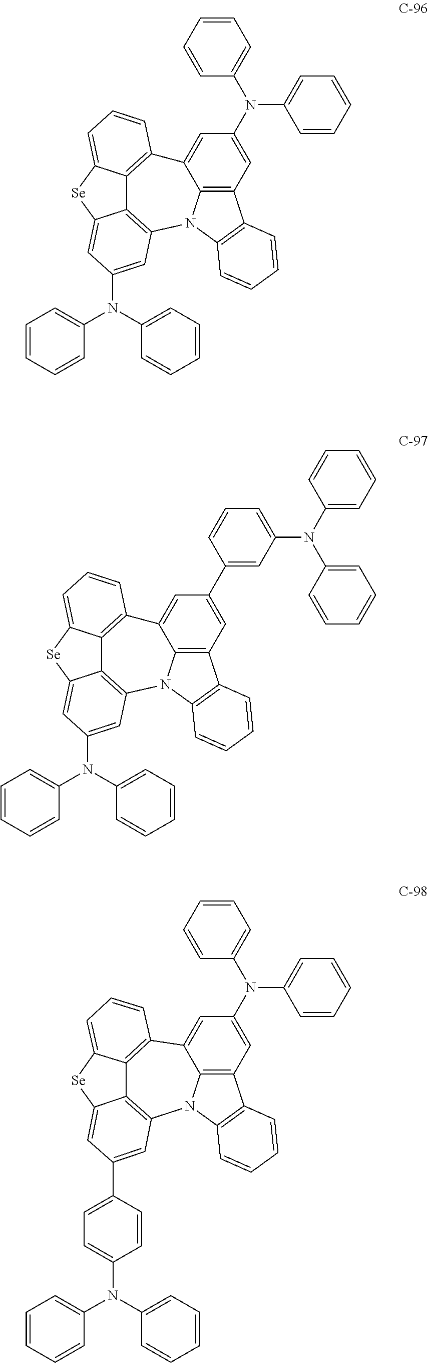 [SMILES: c1ccc(N(c2ccccc2)c2cc3c4cccc5[se]c6cc(N(c7ccccc7)c7ccccc7)cc(c6c54)n4c5ccccc5c(c2)c34)cc1 ; c1ccc(N(c2ccccc2)c2cccc(-c3cc4c5cccc6[se]c7cc(N(c8ccccc8)c8ccccc8)cc(c7c65)n5c6ccccc6c(c3)c45)c2)cc1 ; c1ccc(N(c2ccccc2)c2ccc(-c3cc4[se]c5cccc6c7cc(N(c8ccccc8)c8ccccc8)cc8c9ccccc9n(c(c3)c4c56)c78)cc2)cc1]

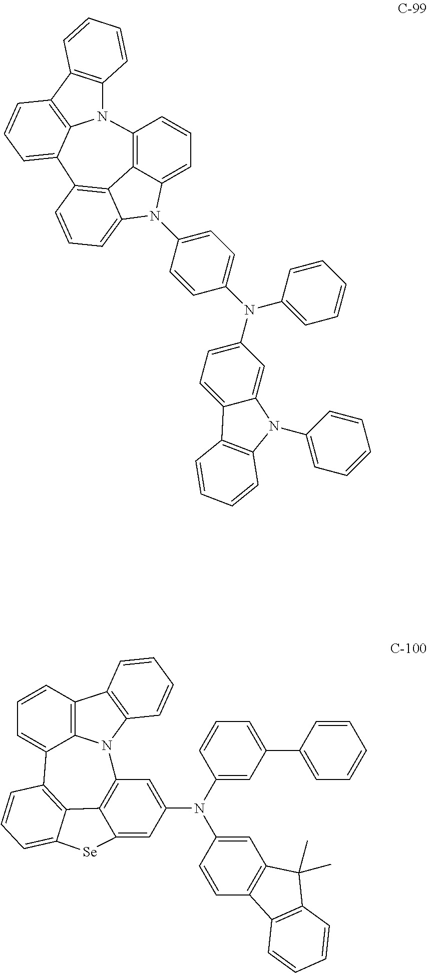 [SMILES: c1ccc(N(c2ccc(-n3c4cccc5c6cccc7c8ccccc8n(c8cccc3c8c54)c67)cc2)c2ccc3c4ccccc4n(-c4ccccc4)c3c2)cc1 ; CC1(C)c2ccccc2-c2ccc(N(c3cccc(-c4ccccc4)c3)c3cc4[se]c5cccc6c7cccc8c9ccccc9n(c(c3)c4c56)c78)cc21]

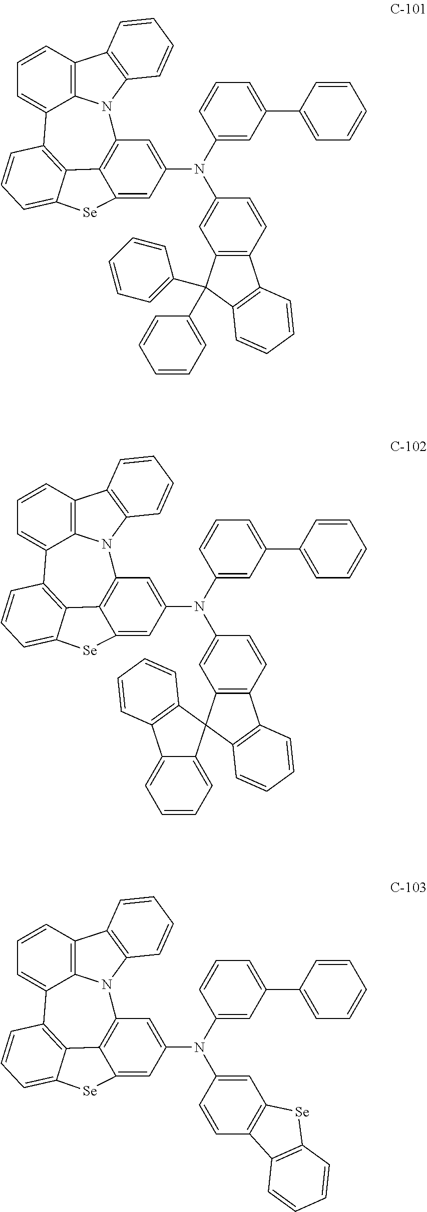 [SMILES: c1ccc(-c2cccc(N(c3ccc4c(c3)C(c3ccccc3)(c3ccccc3)c3ccccc3-4)c3cc4[se]c5cccc6c7cccc8c9ccccc9n(c(c3)c4c56)c78)c2)cc1 ; c1ccc(-c2cccc(N(c3ccc4c(c3)C3(c5ccccc5-c5ccccc53)c3ccccc3-4)c3cc4[se]c5cccc6c7cccc8c9ccccc9n(c(c3)c4c56)c78)c2)cc1 ; c1ccc(-c2cccc(N(c3ccc4c(c3)[se]c3ccccc34)c3cc4[se]c5cccc6c7cccc8c9ccccc9n(c(c3)c4c56)c78)c2)cc1]

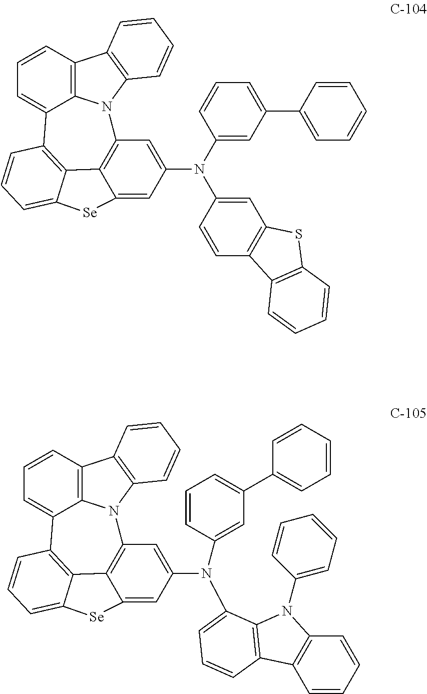 [SMILES: c1ccc(-c2cccc(N(c3ccc4c(c3)sc3ccccc34)c3cc4[se]c5cccc6c7cccc8c9ccccc9n(c(c3)c4c56)c78)c2)cc1 ; c1ccc(-c2cccc(N(c3cc4[se]c5cccc6c7cccc8c9ccccc9n(c(c3)c4c56)c78)c3cccc4c5ccccc5n(-c5ccccc5)c34)c2)cc1]

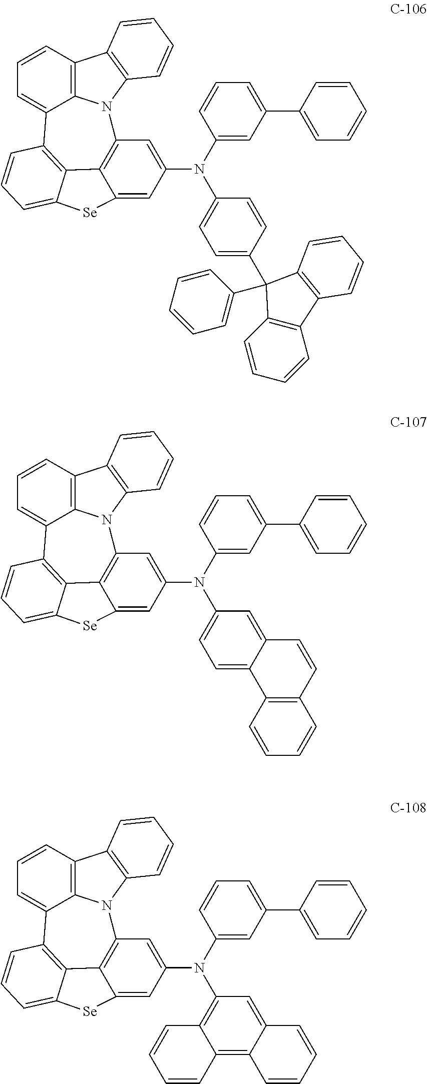 [SMILES: c1ccc(-c2cccc(N(c3ccc(C4(c5ccccc5)c5ccccc5-c5ccccc54)cc3)c3cc4[se]c5cccc6c7cccc8c9ccccc9n(c(c3)c4c56)c78)c2)cc1 ; c1ccc(-c2cccc(N(c3ccc4c(ccc5ccccc54)c3)c3cc4[se]c5cccc6c7cccc8c9ccccc9n(c(c3)c4c56)c78)c2)cc1 ; c1ccc(-c2cccc(N(c3cc4[se]c5cccc6c7cccc8c9ccccc9n(c(c3)c4c56)c78)c3cc4ccccc4c4ccccc34)c2)cc1]

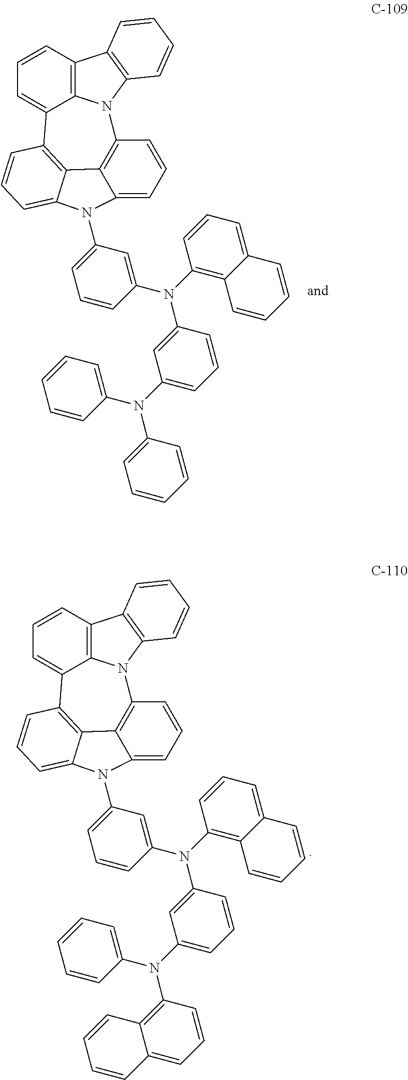 [SMILES: c1ccc(N(c2ccccc2)c2cccc(N(c3cccc(-n4c5cccc6c7cccc8c9ccccc9n(c9cccc4c9c65)c78)c3)c3cccc4ccccc34)c2)cc1 ; c1ccc(N(c2cccc(N(c3cccc(-n4c5cccc6c7cccc8c9ccccc9n(c9cccc4c9c65)c78)c3)c3cccc4ccccc34)c2)c2cccc3ccccc23)cc1]

6. The plurality of host materials according to claim 1, wherein the compound represented by formula 2 is at least one selected from the group consisting of the following compounds:

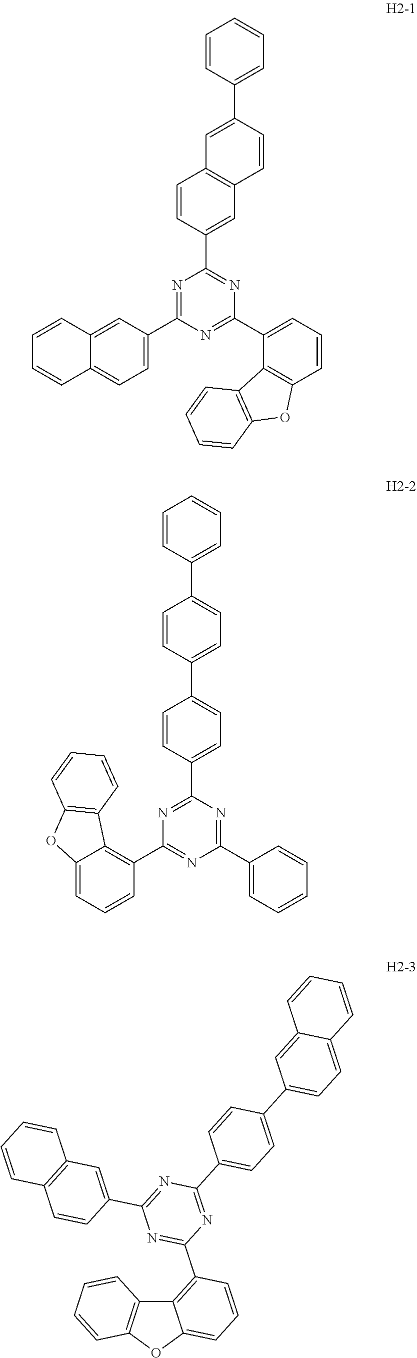 [SMILES: c1ccc(-c2ccc3cc(-c4nc(-c5ccc6ccccc6c5)nc(-c5cccc6oc7ccccc7c56)n4)ccc3c2)cc1 ; c1ccc(-c2ccc(-c3ccc(-c4nc(-c5ccccc5)nc(-c5cccc6oc7ccccc7c56)n4)cc3)cc2)cc1 ; c1ccc2cc(-c3ccc(-c4nc(-c5ccc6ccccc6c5)nc(-c5cccc6oc7ccccc7c56)n4)cc3)ccc2c1]

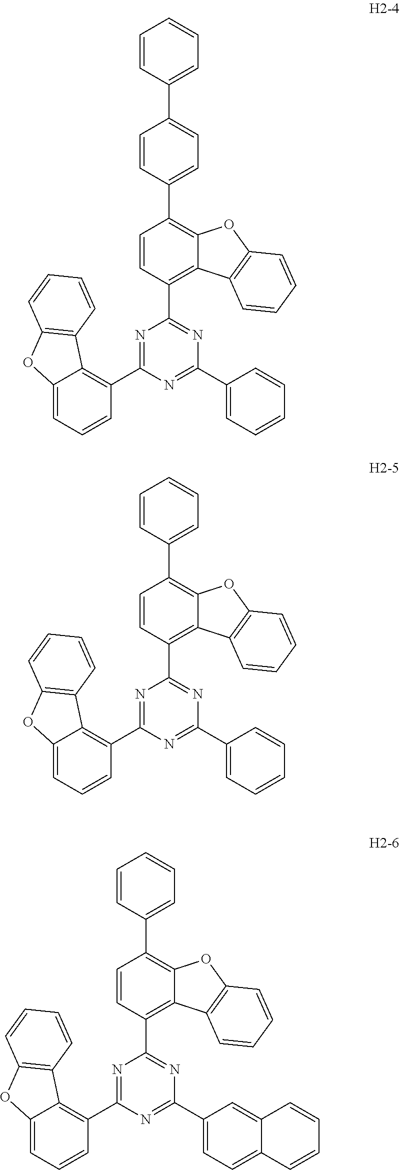 [SMILES: c1ccc(-c2ccc(-c3ccc(-c4nc(-c5ccccc5)nc(-c5cccc6oc7ccccc7c56)n4)c4c3oc3ccccc34)cc2)cc1 ; c1ccc(-c2nc(-c3cccc4oc5ccccc5c34)nc(-c3ccc(-c4ccccc4)c4oc5ccccc5c34)n2)cc1 ; c1ccc(-c2ccc(-c3nc(-c4ccc5ccccc5c4)nc(-c4cccc5oc6ccccc6c45)n3)c3c2oc2ccccc23)cc1]

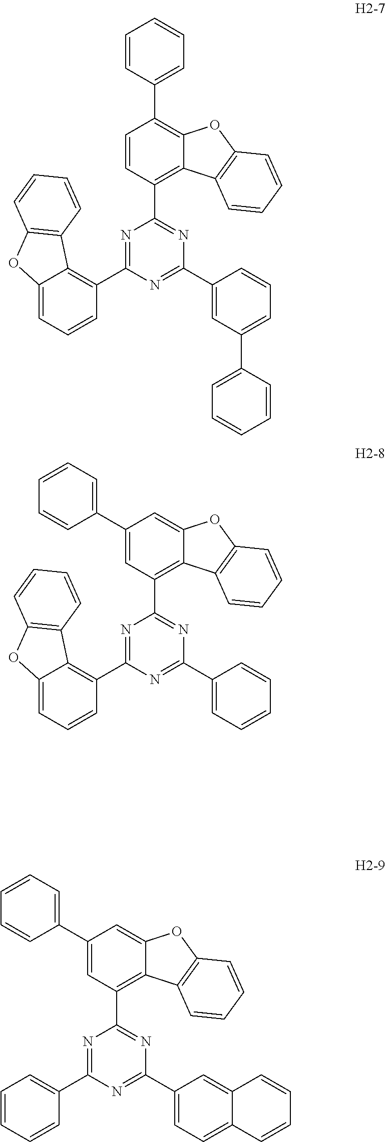 [SMILES: c1ccc(-c2cccc(-c3nc(-c4cccc5oc6ccccc6c45)nc(-c4ccc(-c5ccccc5)c5oc6ccccc6c45)n3)c2)cc1 ; c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccccc4)nc(-c4ccc5ccccc5c4)n3)c3c(c2)oc2ccccc23)cc1]

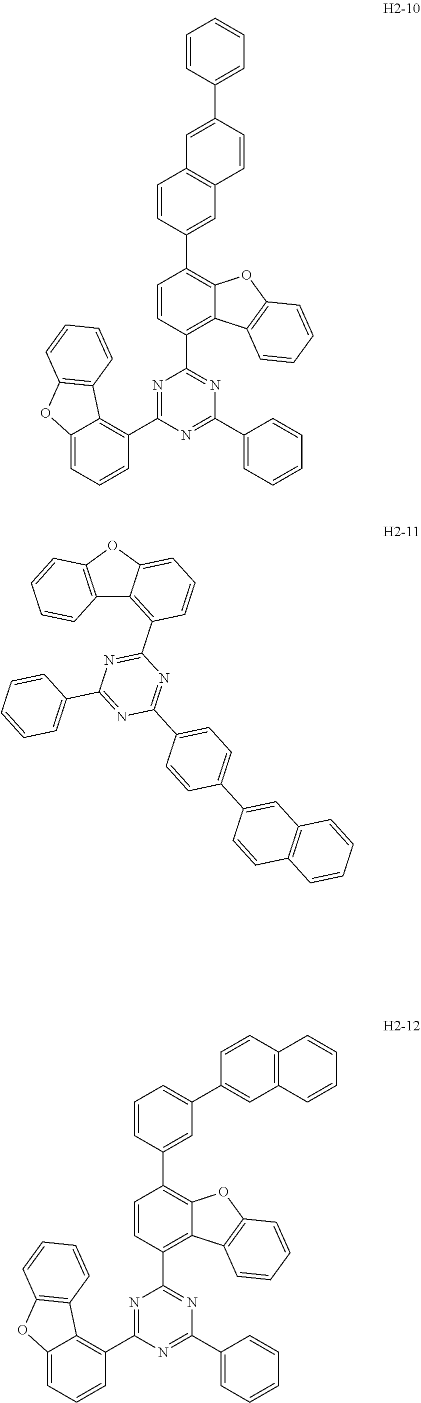 [SMILES: c1ccc(-c2ccc3cc(-c4ccc(-c5nc(-c6ccccc6)nc(-c6cccc7oc8ccccc8c67)n5)c5c4oc4ccccc45)ccc3c2)cc1 ; c1ccc(-c2nc(-c3ccc(-c4ccc5ccccc5c4)cc3)nc(-c3cccc4oc5ccccc5c34)n2)cc1 ; c1ccc(-c2nc(-c3cccc4oc5ccccc5c34)nc(-c3ccc(-c4cccc(-c5ccc6ccccc6c5)c4)c4oc5ccccc5c34)n2)cc1]

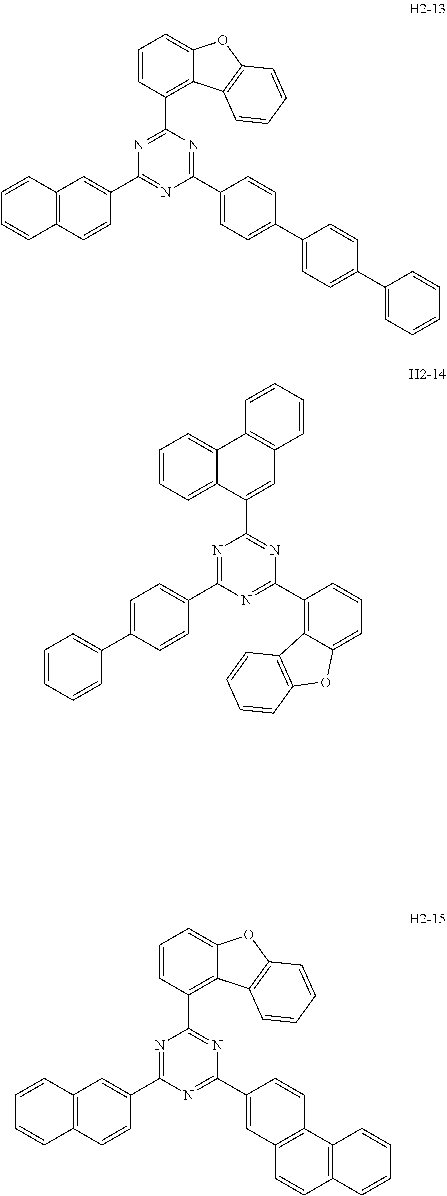 [SMILES: c1ccc(-c2ccc(-c3ccc(-c4nc(-c5ccc6ccccc6c5)nc(-c5cccc6oc7ccccc7c56)n4)cc3)cc2)cc1 ; c1ccc(-c2ccc(-c3nc(-c4cc5ccccc5c5ccccc45)nc(-c4cccc5oc6ccccc6c45)n3)cc2)cc1 ; c1ccc2cc(-c3nc(-c4ccc5c(ccc6ccccc65)c4)nc(-c4cccc5oc6ccccc6c45)n3)ccc2c1]

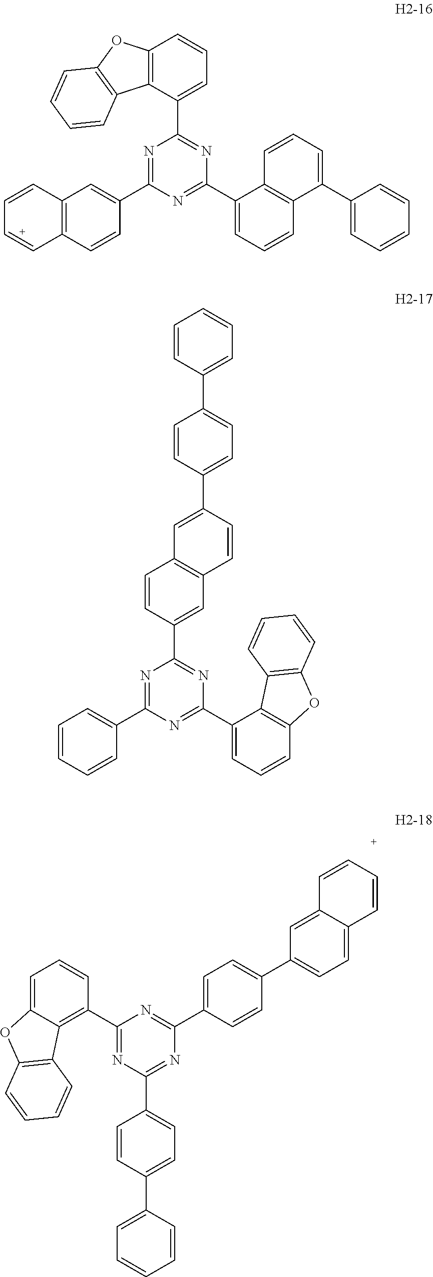 [SMILES: c1ccc(-c2cccc3c(-c4nc(-c5ccc6ccccc6c5)nc(-c5cccc6oc7ccccc7c56)n4)cccc23)cc1 ; c1ccc(-c2ccc(-c3ccc4cc(-c5nc(-c6ccccc6)nc(-c6cccc7oc8ccccc8c67)n5)ccc4c3)cc2)cc1 ; c1ccc(-c2ccc(-c3nc(-c4ccc(-c5ccc6ccccc6c5)cc4)nc(-c4cccc5oc6ccccc6c45)n3)cc2)cc1]

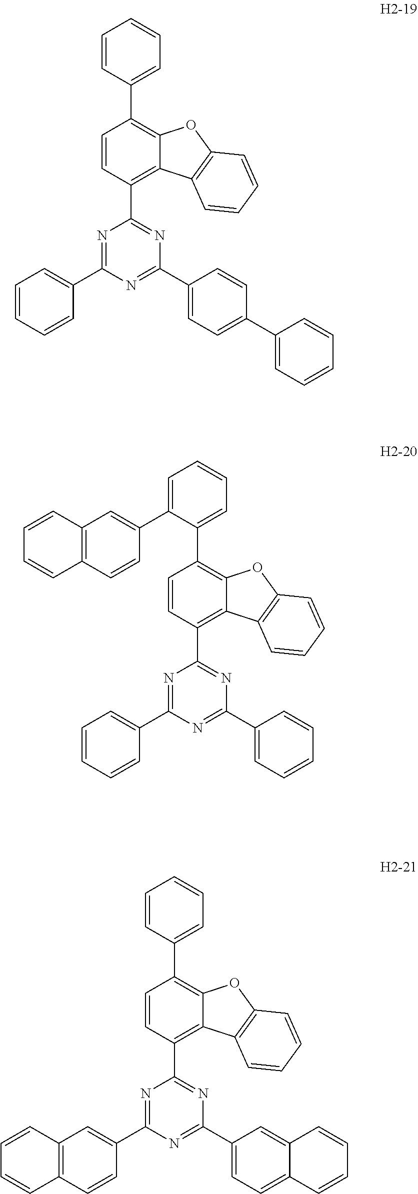 [SMILES: c1ccc(-c2ccc(-c3nc(-c4ccccc4)nc(-c4ccc(-c5ccccc5)c5oc6ccccc6c45)n3)cc2)cc1 ; c1ccc(-c2nc(-c3ccccc3)nc(-c3ccc(-c4ccccc4-c4ccc5ccccc5c4)c4oc5ccccc5c34)n2)cc1 ; c1ccc(-c2ccc(-c3nc(-c4ccc5ccccc5c4)nc(-c4ccc5ccccc5c4)n3)c3c2oc2ccccc23)cc1]

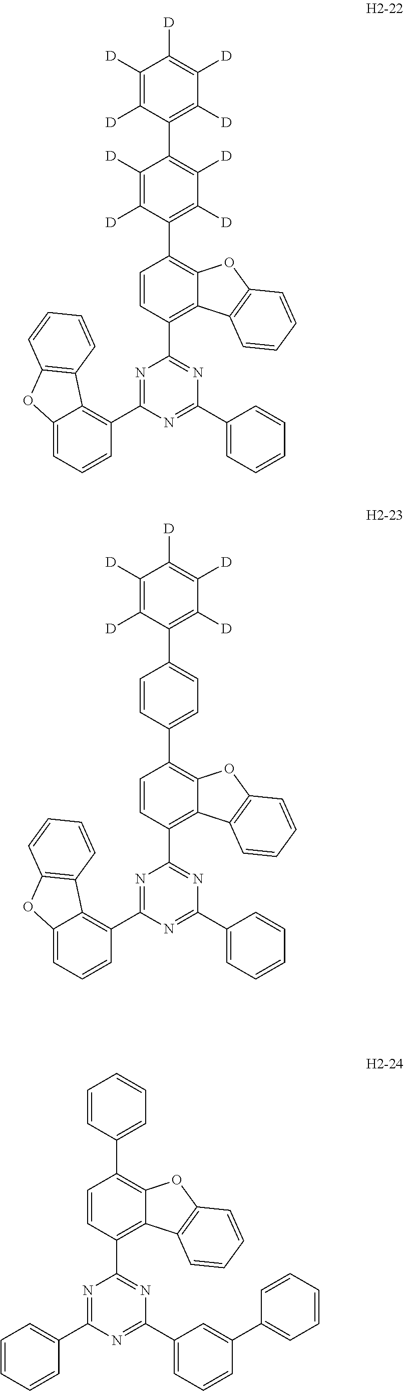 [SMILES: [2H]c1c([2H])c([2H])c(-c2c([2H])c([2H])c(-c3ccc(-c4nc(-c5ccccc5)nc(-c5cccc6oc7ccccc7c56)n4)c4c3oc3ccccc34)c([2H])c2[2H])c([2H])c1[2H] ; [2H]c1c([2H])c([2H])c(-c2ccc(-c3ccc(-c4nc(-c5ccccc5)nc(-c5cccc6oc7ccccc7c56)n4)c4c3oc3ccccc34)cc2)c([2H])c1[2H] ; c1ccc(-c2cccc(-c3nc(-c4ccccc4)nc(-c4ccc(-c5ccccc5)c5oc6ccccc6c45)n3)c2)cc1]

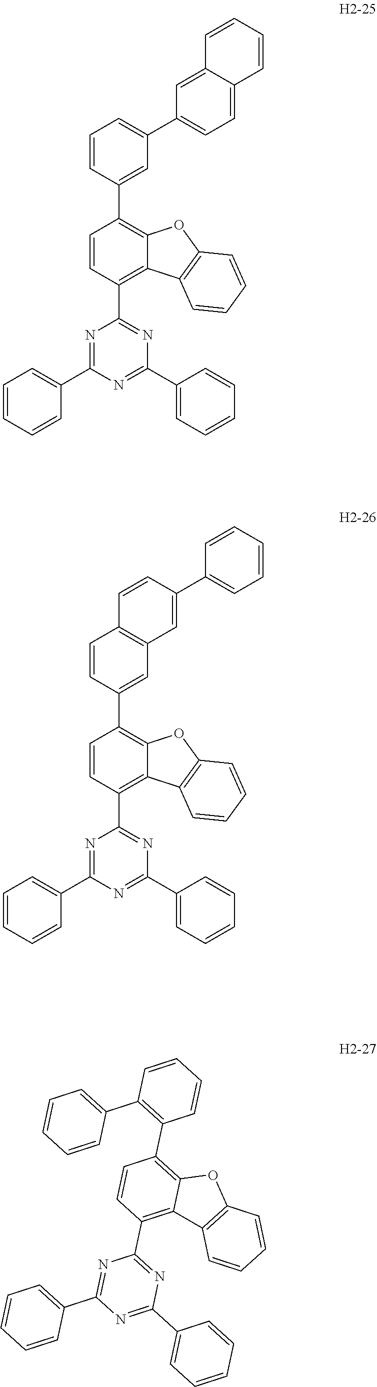 [SMILES: c1ccc(-c2nc(-c3ccccc3)nc(-c3ccc(-c4cccc(-c5ccc6ccccc6c5)c4)c4oc5ccccc5c34)n2)cc1 ; c1ccc(-c2ccc3ccc(-c4ccc(-c5nc(-c6ccccc6)nc(-c6ccccc6)n5)c5c4oc4ccccc45)cc3c2)cc1 ; c1ccc(-c2nc(-c3ccccc3)nc(-c3ccc(-c4ccccc4-c4ccccc4)c4oc5ccccc5c34)n2)cc1]

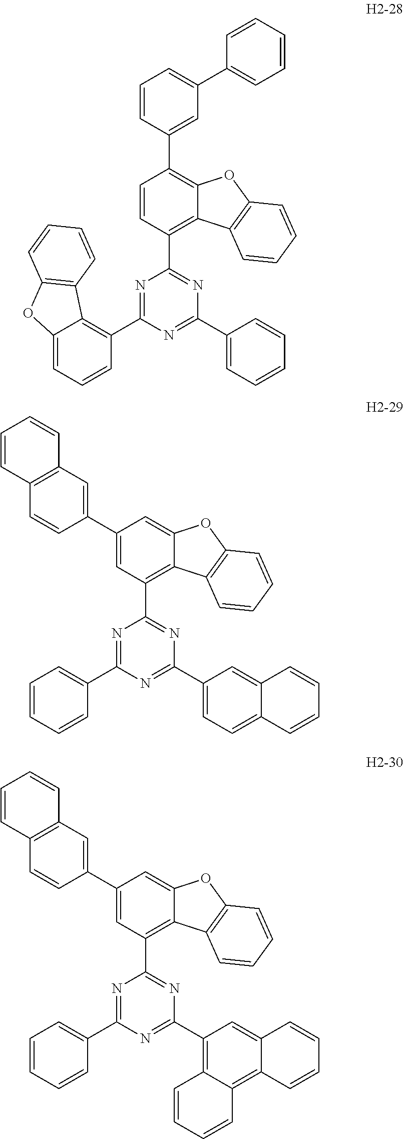 [SMILES: c1ccc(-c2cccc(-c3ccc(-c4nc(-c5ccccc5)nc(-c5cccc6oc7ccccc7c56)n4)c4c3oc3ccccc34)c2)cc1 ; c1ccc(-c2nc(-c3ccc4ccccc4c3)nc(-c3cc(-c4ccc5ccccc5c4)cc4oc5ccccc5c34)n2)cc1 ; c1ccc(-c2nc(-c3cc4ccccc4c4ccccc34)nc(-c3cc(-c4ccc5ccccc5c4)cc4oc5ccccc5c34)n2)cc1]

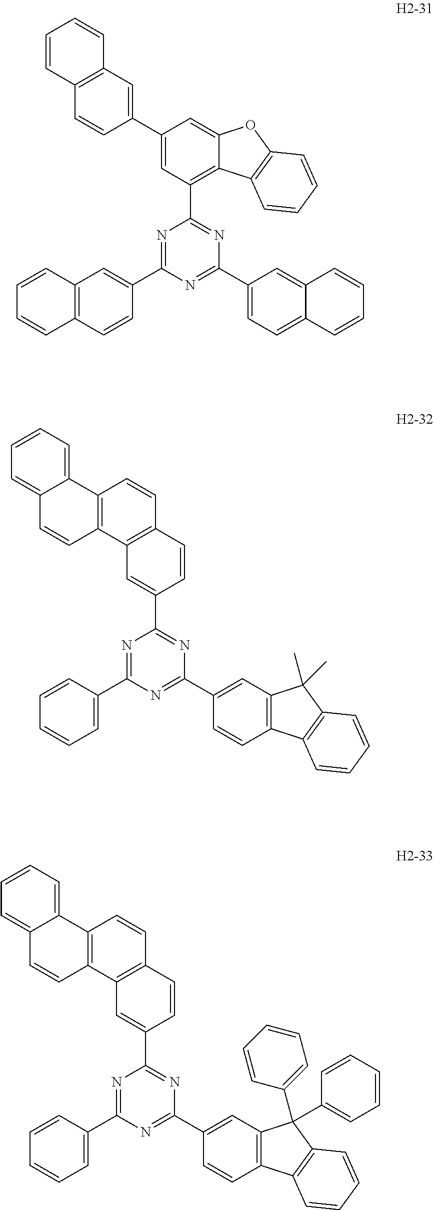 [SMILES: c1ccc2cc(-c3cc(-c4nc(-c5ccc6ccccc6c5)nc(-c5ccc6ccccc6c5)n4)c4c(c3)oc3ccccc34)ccc2c1 ; CC1(C)c2ccccc2-c2ccc(-c3nc(-c4ccccc4)nc(-c4ccc5ccc6c7ccccc7ccc6c5c4)n3)cc21 ; c1ccc(-c2nc(-c3ccc4c(c3)C(c3ccccc3)(c3ccccc3)c3ccccc3-4)nc(-c3ccc4ccc5c6ccccc6ccc5c4c3)n2)cc1]

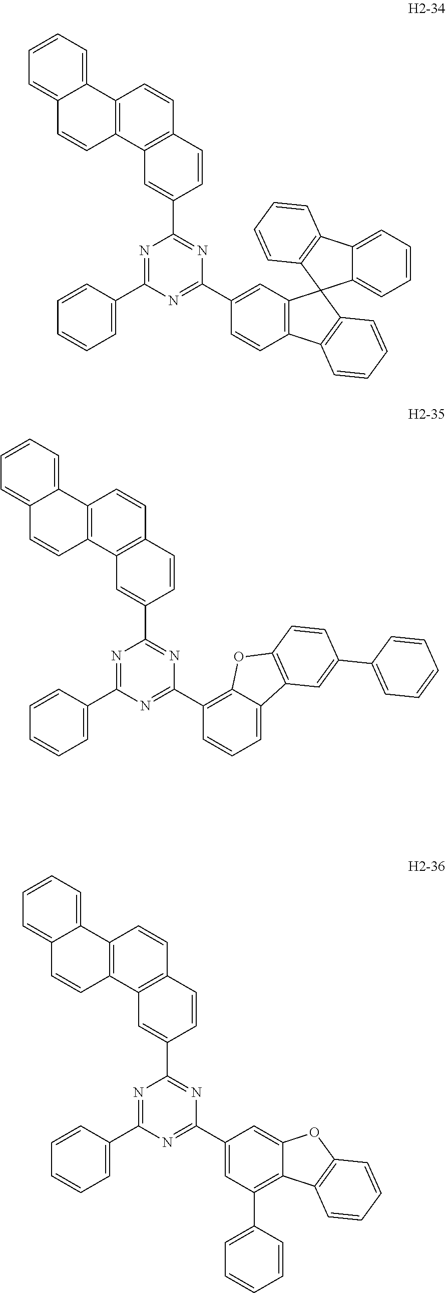 [SMILES: c1ccc(-c2nc(-c3ccc4c(c3)C3(c5ccccc5-c5ccccc53)c3ccccc3-4)nc(-c3ccc4ccc5c6ccccc6ccc5c4c3)n2)cc1 ; c1ccc(-c2ccc3oc4c(-c5nc(-c6ccccc6)nc(-c6ccc7ccc8c9ccccc9ccc8c7c6)n5)cccc4c3c2)cc1 ; c1ccc(-c2nc(-c3cc(-c4ccccc4)c4c(c3)oc3ccccc34)nc(-c3ccc4ccc5c6ccccc6ccc5c4c3)n2)cc1]

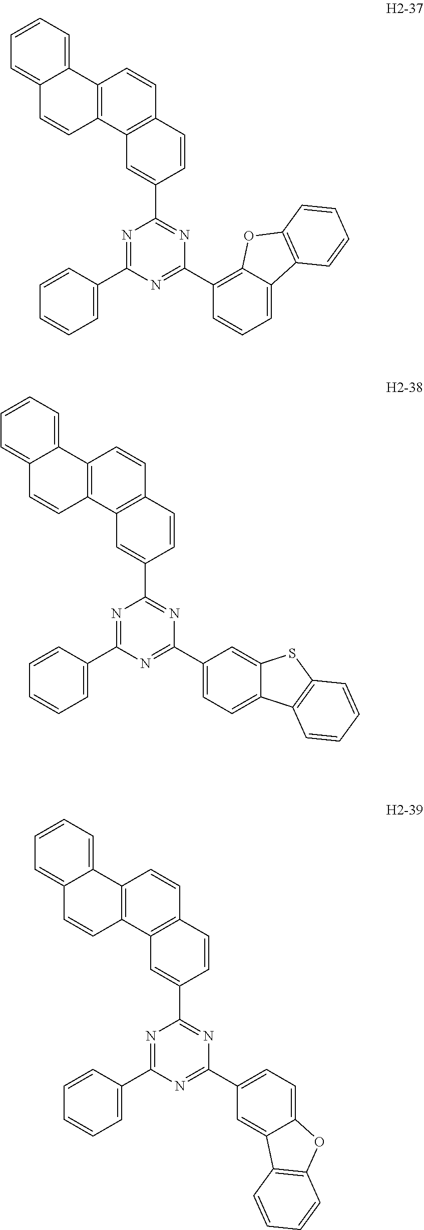 [SMILES: c1ccc(-c2nc(-c3ccc4ccc5c6ccccc6ccc5c4c3)nc(-c3cccc4c3oc3ccccc34)n2)cc1 ; c1ccc(-c2nc(-c3ccc4c(c3)sc3ccccc34)nc(-c3ccc4ccc5c6ccccc6ccc5c4c3)n2)cc1 ; c1ccc(-c2nc(-c3ccc4ccc5c6ccccc6ccc5c4c3)nc(-c3ccc4oc5ccccc5c4c3)n2)cc1]

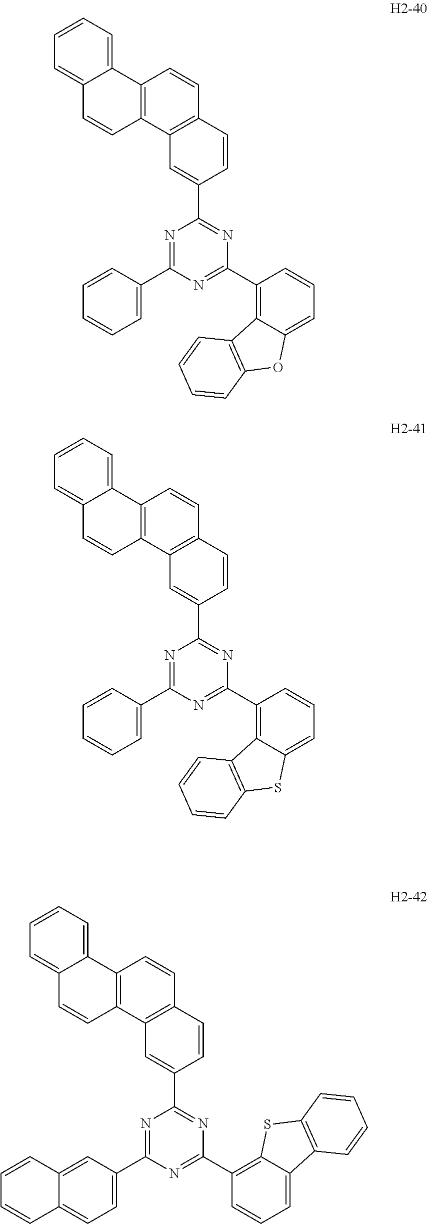 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1 ; c1ccc2cc(-c3nc(-c4ccc5ccc6c7ccccc7ccc6c5c4)nc(-c4cccc5c4sc4ccccc45)n3)ccc2c1]

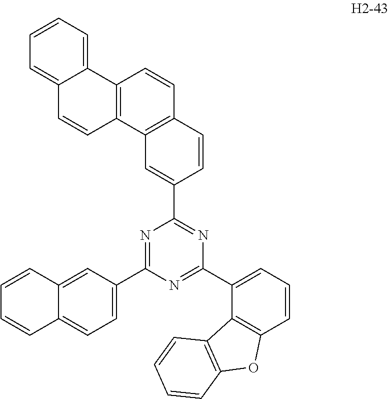 [SMILES: c1ccc2cc(-c3nc(-c4ccc5ccc6c7ccccc7ccc6c5c4)nc(-c4cccc5oc6ccccc6c45)n3)ccc2c1]

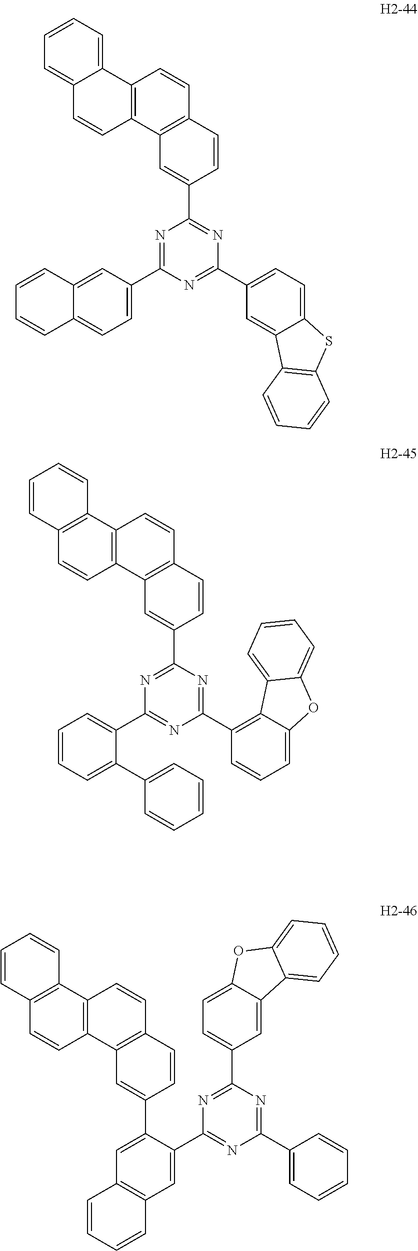 [SMILES: c1ccc2cc(-c3nc(-c4ccc5ccc6c7ccccc7ccc6c5c4)nc(-c4ccc5sc6ccccc6c5c4)n3)ccc2c1 ; c1ccc(-c2ccccc2-c2nc(-c3ccc4ccc5c6ccccc6ccc5c4c3)nc(-c3cccc4oc5ccccc5c34)n2)cc1 ; c1ccc(-c2nc(-c3ccc4oc5ccccc5c4c3)nc(-c3cc4ccccc4cc3-c3ccc4ccc5c6ccccc6ccc5c4c3)n2)cc1]

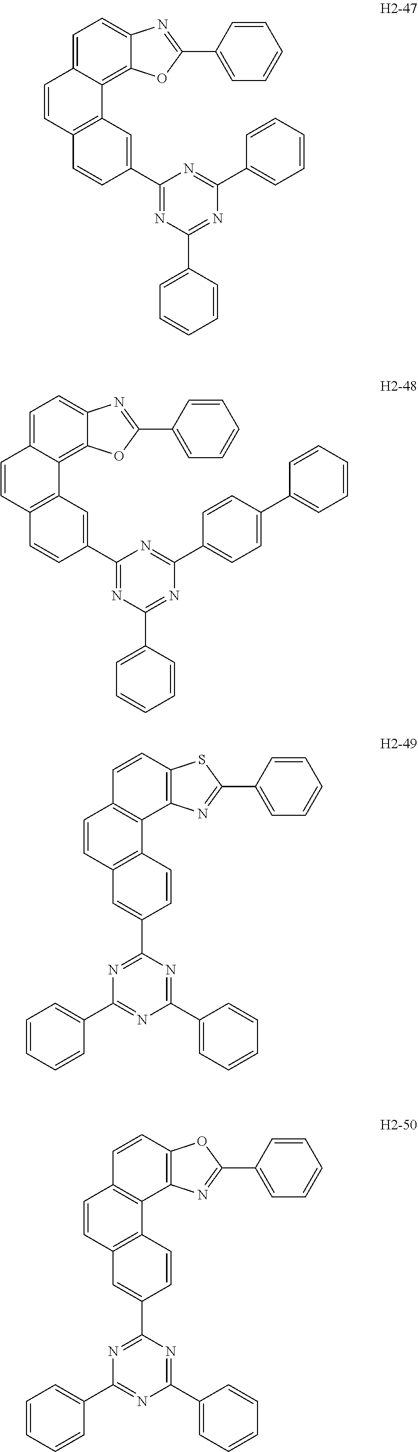

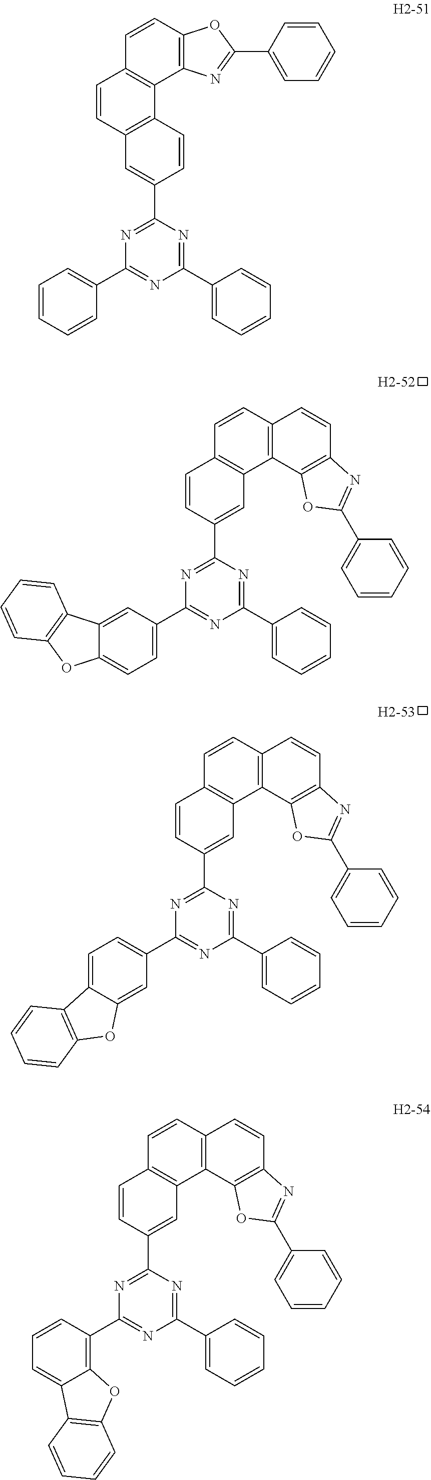 [SMILES: c1ccc(-c2nc(-c3ccccc3)nc(-c3ccc4c(ccc5ccc6oc(-c7ccccc7)nc6c54)c3)n2)cc1 ; c1ccc(-c2nc(-c3ccc4oc5ccccc5c4c3)nc(-c3ccc4ccc5ccc6nc(-c7ccccc7)oc6c5c4c3)n2)cc1 ; c1ccc(-c2nc(-c3ccc4c(c3)oc3ccccc34)nc(-c3ccc4ccc5ccc6nc(-c7ccccc7)oc6c5c4c3)n2)cc1 ; c1ccc(-c2nc(-c3ccc4ccc5ccc6nc(-c7ccccc7)oc6c5c4c3)nc(-c3cccc4c3oc3ccccc34)n2)cc1]

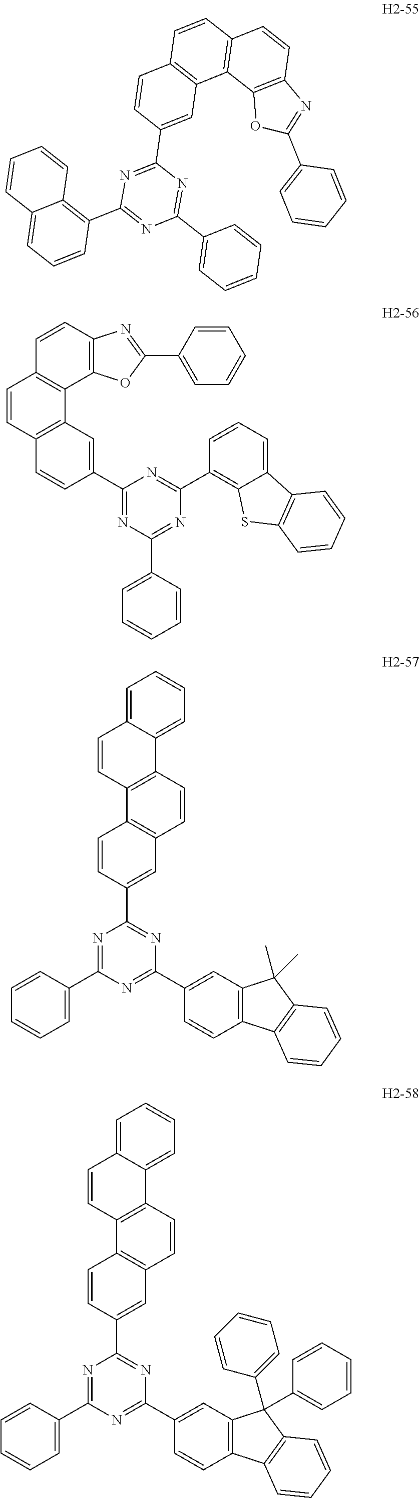

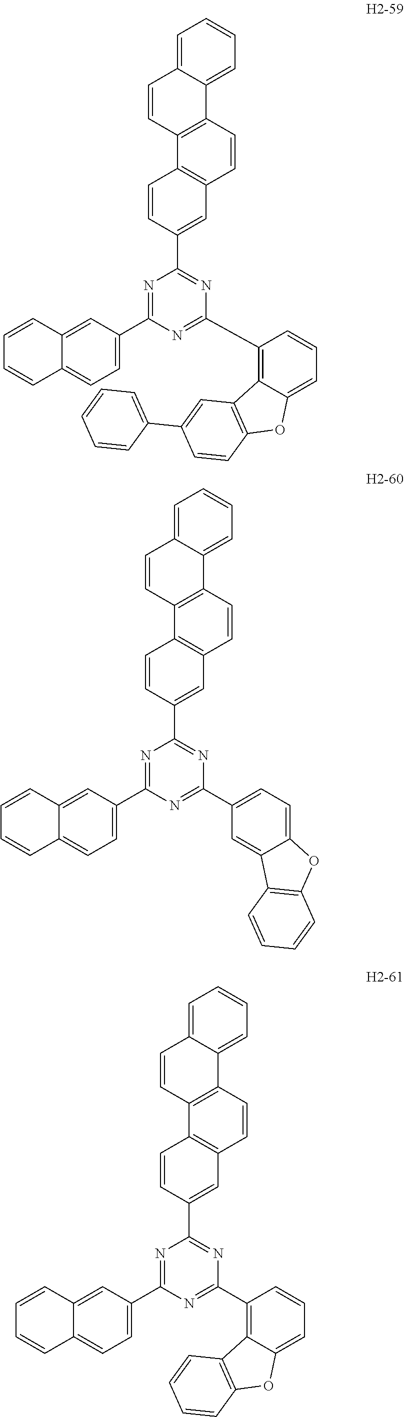

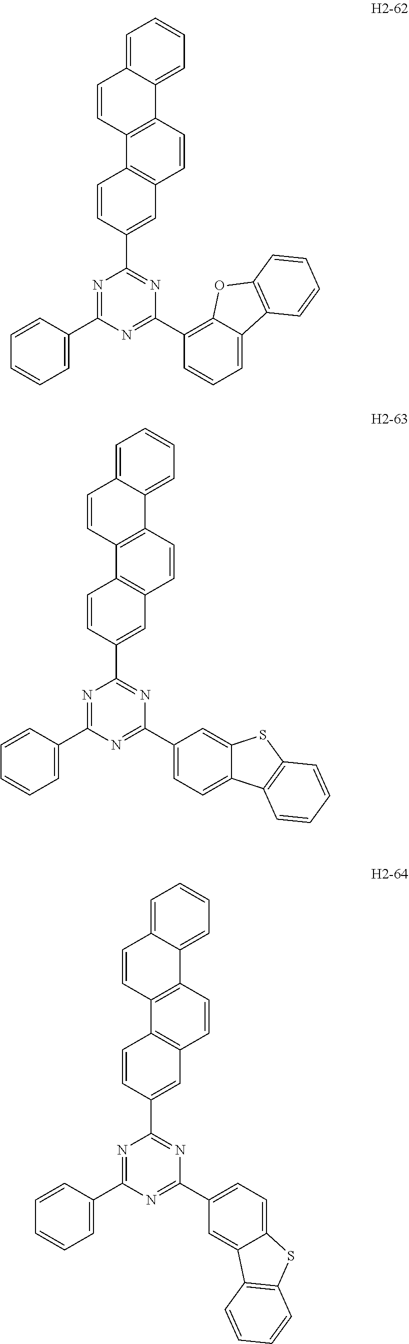 [SMILES: c1ccc(-c2nc(-c3ccc4c(ccc5c6ccccc6ccc45)c3)nc(-c3cccc4c3oc3ccccc34)n2)cc1 ; c1ccc(-c2nc(-c3ccc4c(ccc5c6ccccc6ccc45)c3)nc(-c3ccc4c(c3)sc3ccccc34)n2)cc1 ; c1ccc(-c2nc(-c3ccc4c(ccc5c6ccccc6ccc45)c3)nc(-c3ccc4sc5ccccc5c4c3)n2)cc1]

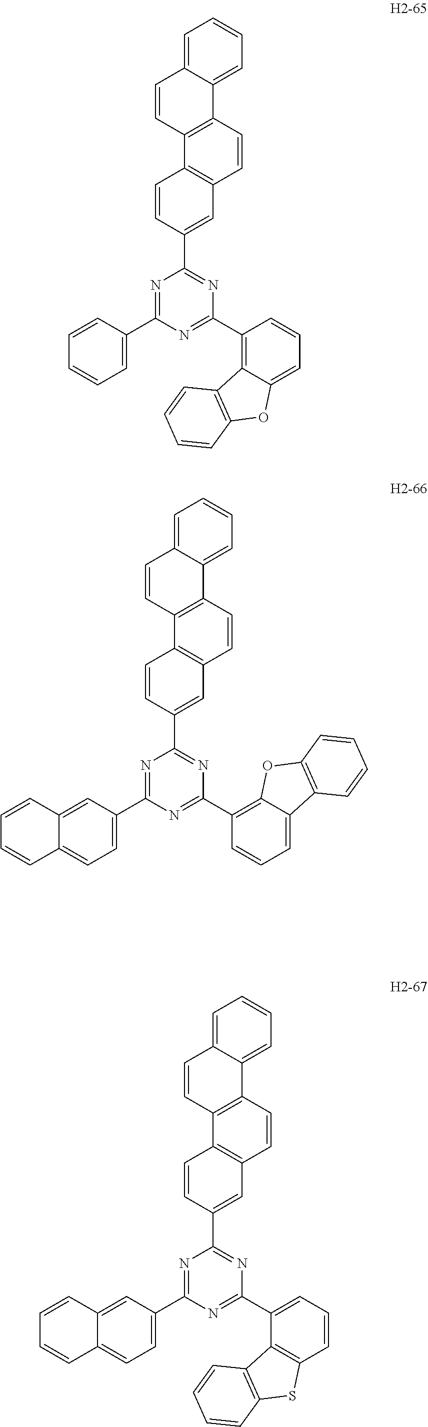 [SMILES: c1ccc(-c2nc(-c3ccc4c(ccc5c6ccccc6ccc45)c3)nc(-c3cccc4oc5ccccc5c34)n2)cc1 ; c1ccc2cc(-c3nc(-c4ccc5c(ccc6c7ccccc7ccc56)c4)nc(-c4cccc5c4oc4ccccc45)n3)ccc2c1 ; c1ccc2cc(-c3nc(-c4ccc5c(ccc6c7ccccc7ccc56)c4)nc(-c4cccc5sc6ccccc6c45)n3)ccc2c1]

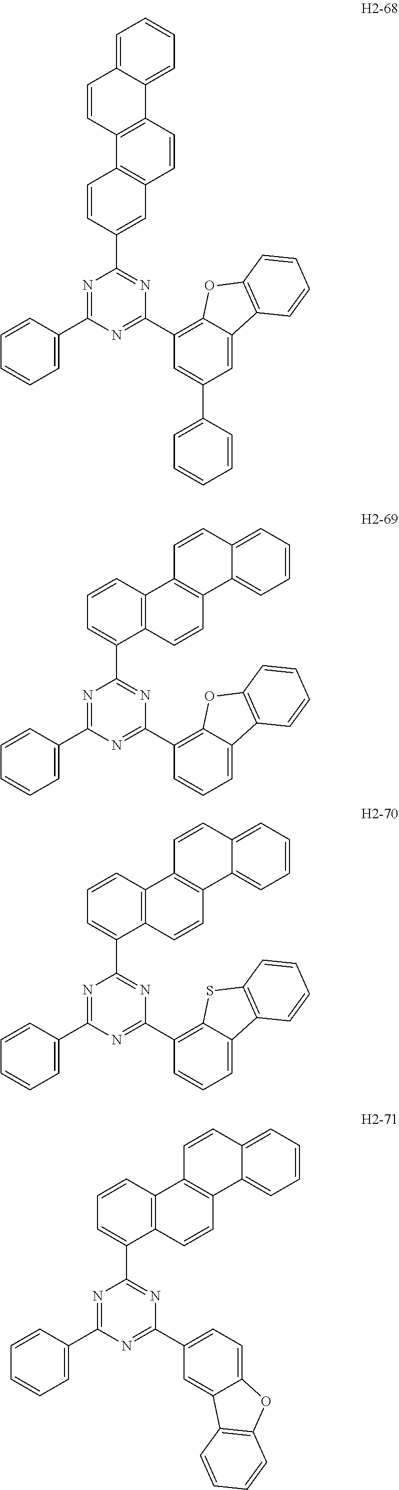

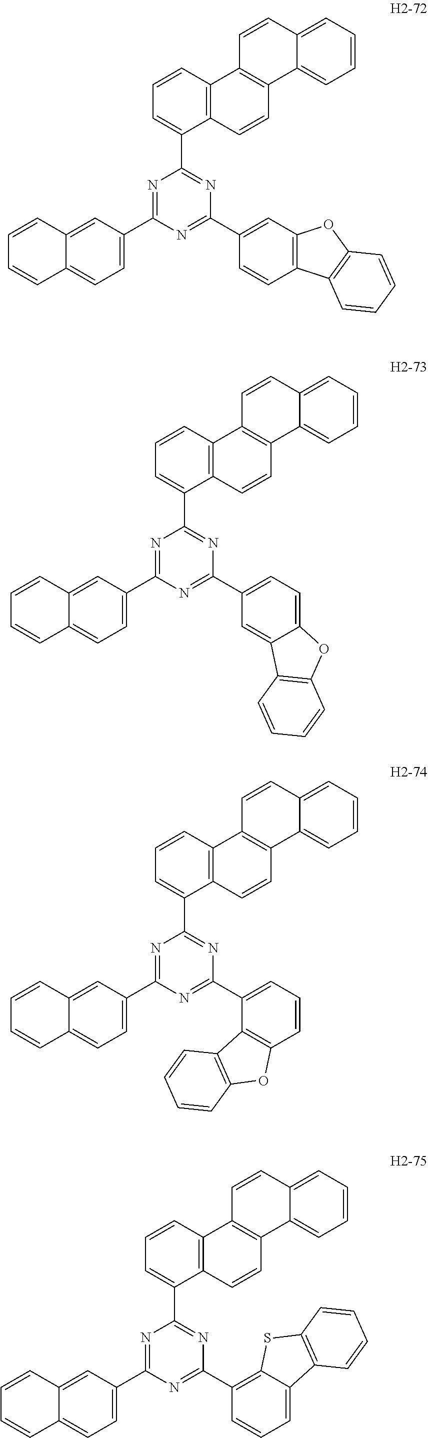 [SMILES: c1ccc2cc(-c3nc(-c4ccc5c(c4)oc4ccccc45)nc(-c4cccc5c4ccc4c6ccccc6ccc54)n3)ccc2c1 ; c1ccc2cc(-c3nc(-c4ccc5oc6ccccc6c5c4)nc(-c4cccc5c4ccc4c6ccccc6ccc54)n3)ccc2c1 ; c1ccc2cc(-c3nc(-c4cccc5c4ccc4c6ccccc6ccc54)nc(-c4cccc5oc6ccccc6c45)n3)ccc2c1 ; c1ccc2cc(-c3nc(-c4cccc5c4ccc4c6ccccc6ccc54)nc(-c4cccc5c4sc4ccccc45)n3)ccc2c1]

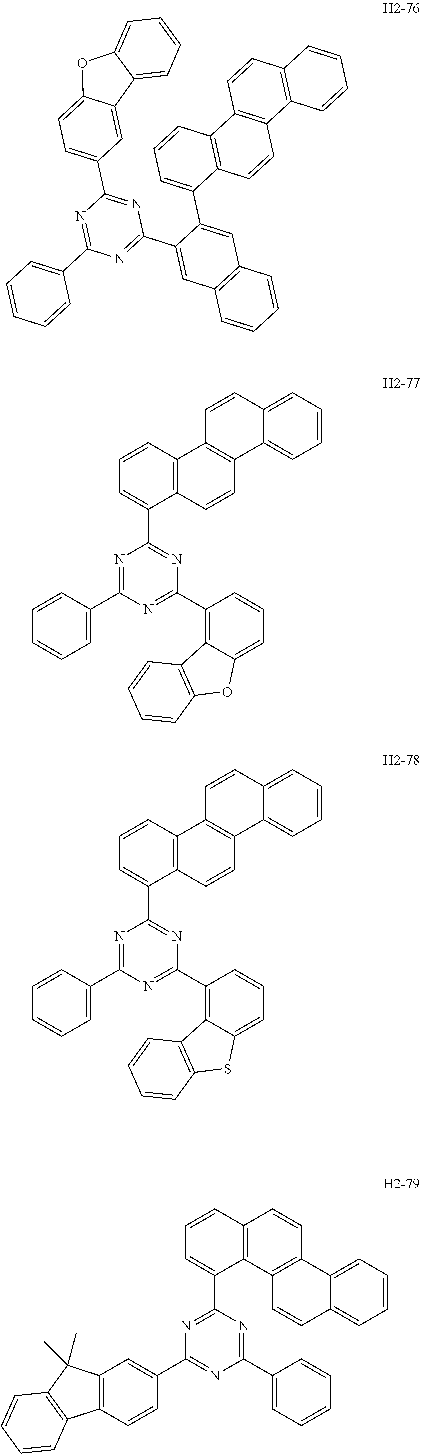 [SMILES: c1ccc(-c2nc(-c3ccc4oc5ccccc5c4c3)nc(-c3cc4ccccc4cc3-c3cccc4c3ccc3c5ccccc5ccc43)n2)cc1 ; c1ccc(-c2nc(-c3cccc4c3ccc3c5ccccc5ccc43)nc(-c3cccc4oc5ccccc5c34)n2)cc1 ; c1ccc(-c2nc(-c3cccc4c3ccc3c5ccccc5ccc43)nc(-c3cccc4sc5ccccc5c34)n2)cc1 ; CC1(C)c2ccccc2-c2ccc(-c3nc(-c4ccccc4)nc(-c4cccc5ccc6c7ccccc7ccc6c45)n3)cc21]

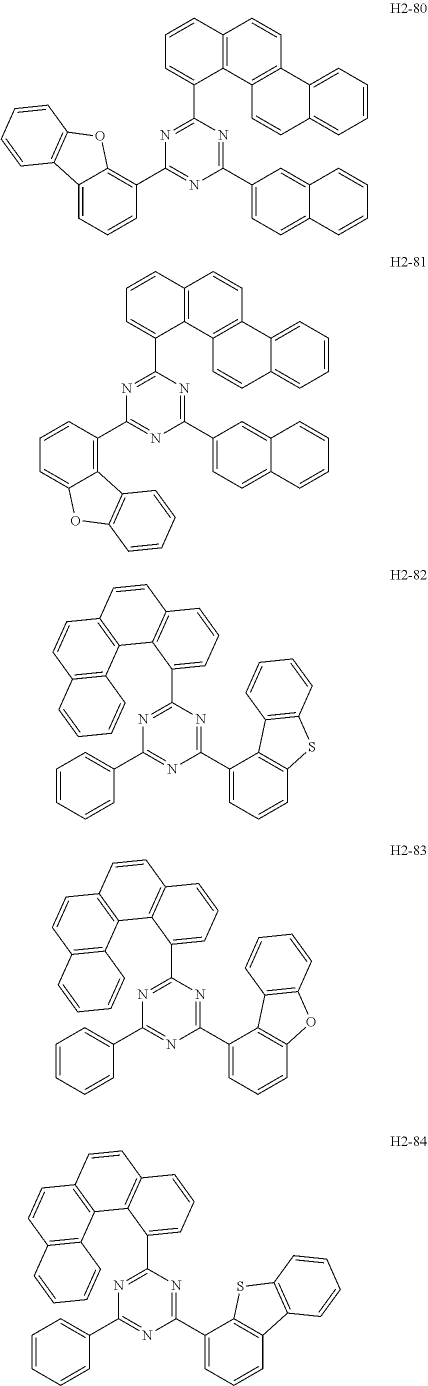

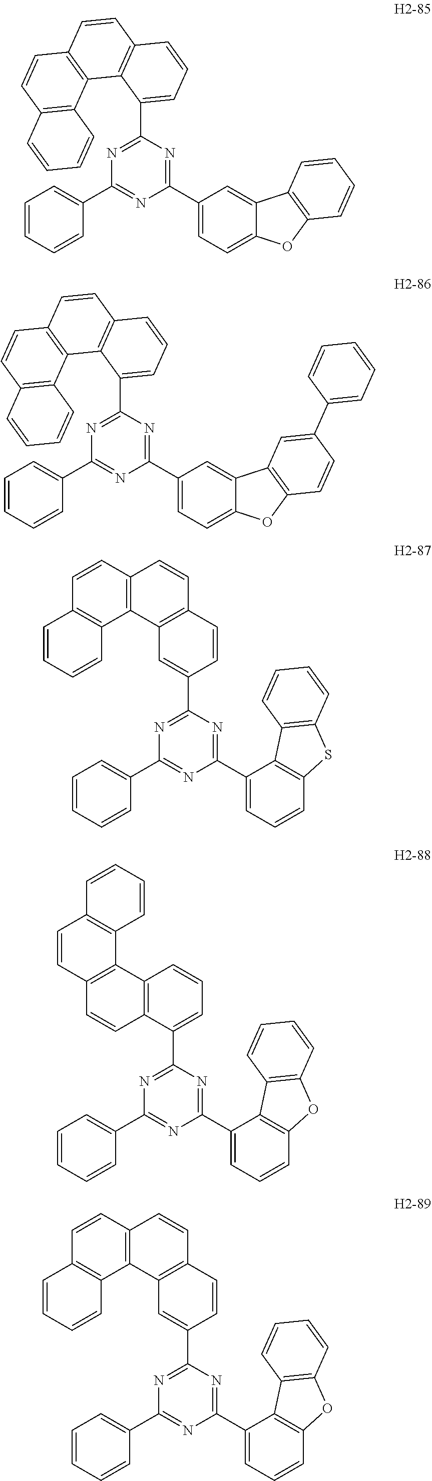

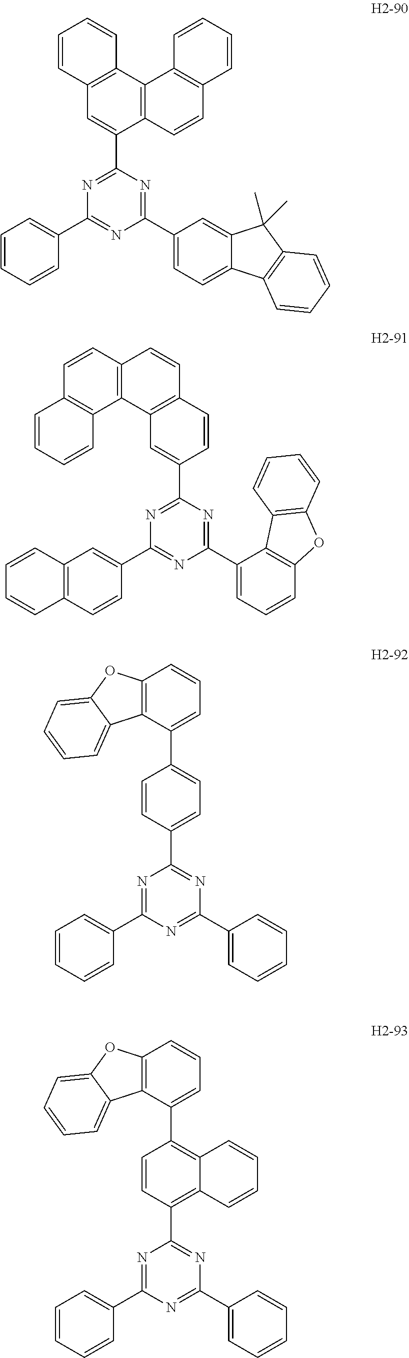 [SMILES: CC1(C)c2ccccc2-c2ccc(-c3nc(-c4ccccc4)nc(-c4cc5ccccc5c5c4ccc4ccccc45)n3)cc21 ; c1ccc2cc(-c3nc(-c4ccc5ccc6ccc7ccccc7c6c5c4)nc(-c4cccc5oc6ccccc6c45)n3)ccc2c1 ; c1ccc(-c2nc(-c3ccccc3)nc(-c3ccc(-c4cccc5oc6ccccc6c45)cc3)n2)cc1 ; c1ccc(-c2nc(-c3ccccc3)nc(-c3ccc(-c4cccc5oc6ccccc6c45)c4ccccc34)n2)cc1]

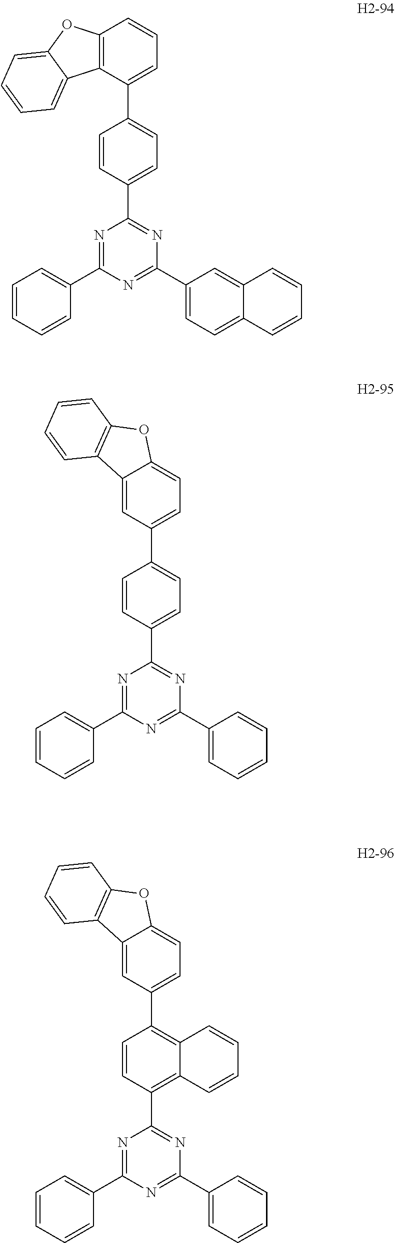 [SMILES: c1ccc(-c2nc(-c3ccc(-c4cccc5oc6ccccc6c45)cc3)nc(-c3ccc4ccccc4c3)n2)cc1 ; c1ccc(-c2nc(-c3ccccc3)nc(-c3ccc(-c4ccc5oc6ccccc6c5c4)cc3)n2)cc1 ; c1ccc(-c2nc(-c3ccccc3)nc(-c3ccc(-c4ccc5oc6ccccc6c5c4)c4ccccc34)n2)cc1]

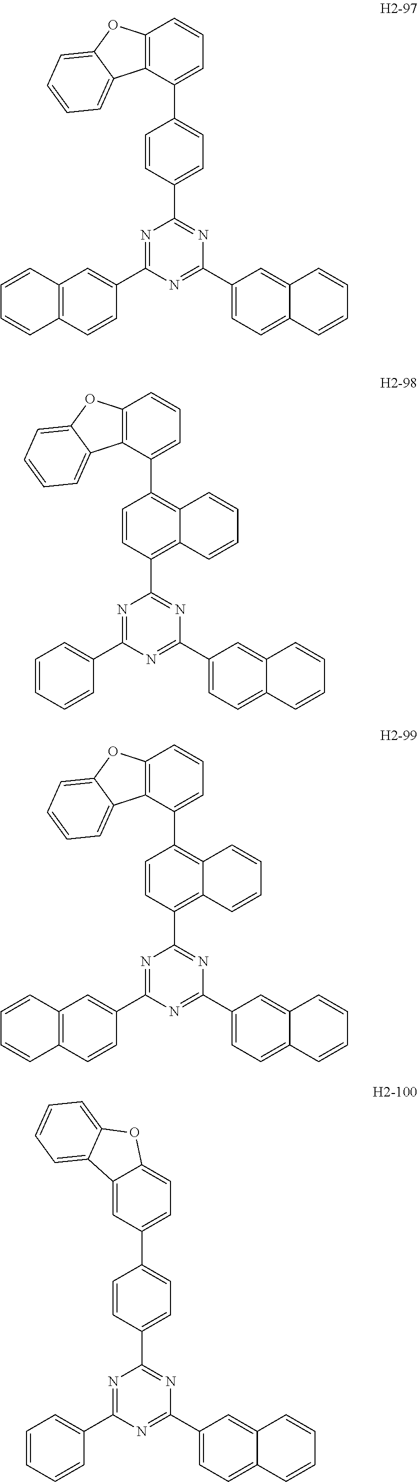

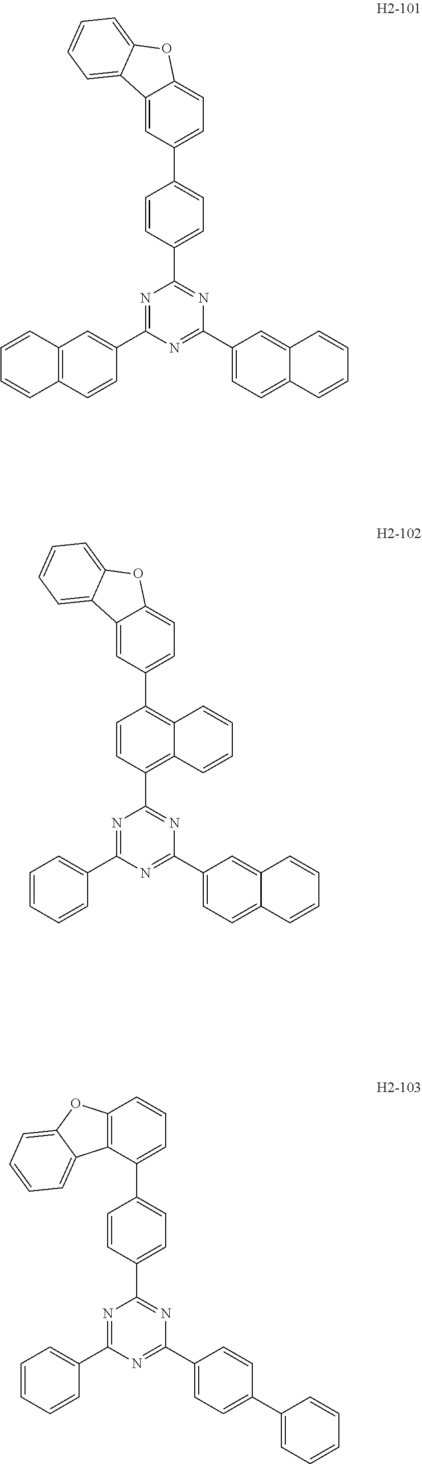 [SMILES: c1ccc2cc(-c3nc(-c4ccc(-c5ccc6oc7ccccc7c6c5)cc4)nc(-c4ccc5ccccc5c4)n3)ccc2c1 ; c1ccc(-c2nc(-c3ccc4ccccc4c3)nc(-c3ccc(-c4ccc5oc6ccccc6c5c4)c4ccccc34)n2)cc1 ; c1ccc(-c2ccc(-c3nc(-c4ccccc4)nc(-c4ccc(-c5cccc6oc7ccccc7c56)cc4)n3)cc2)cc1]

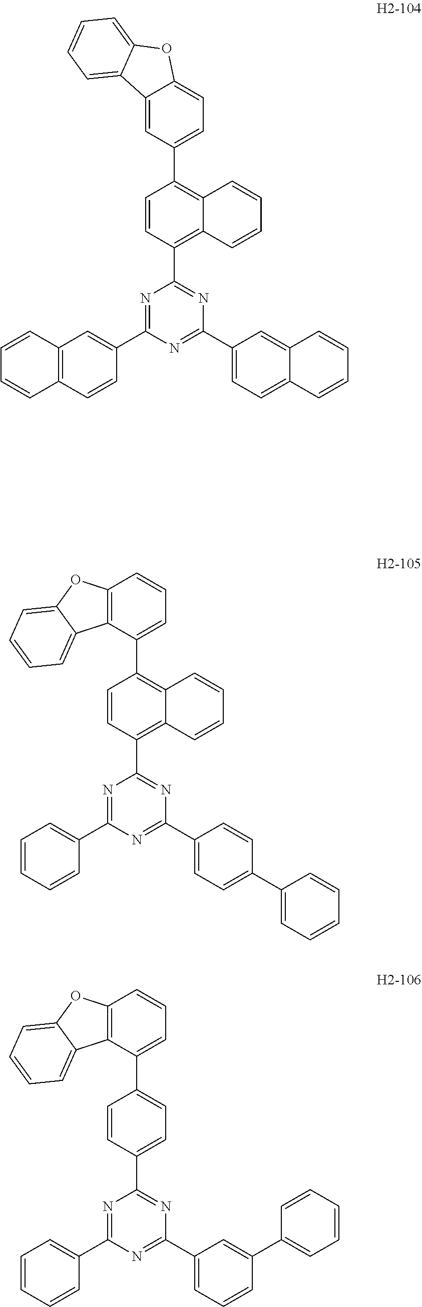 [SMILES: c1ccc2cc(-c3nc(-c4ccc5ccccc5c4)nc(-c4ccc(-c5ccc6oc7ccccc7c6c5)c5ccccc45)n3)ccc2c1 ; c1ccc(-c2ccc(-c3nc(-c4ccccc4)nc(-c4ccc(-c5cccc6oc7ccccc7c56)c5ccccc45)n3)cc2)cc1 ; c1ccc(-c2cccc(-c3nc(-c4ccccc4)nc(-c4ccc(-c5cccc6oc7ccccc7c56)cc4)n3)c2)cc1]

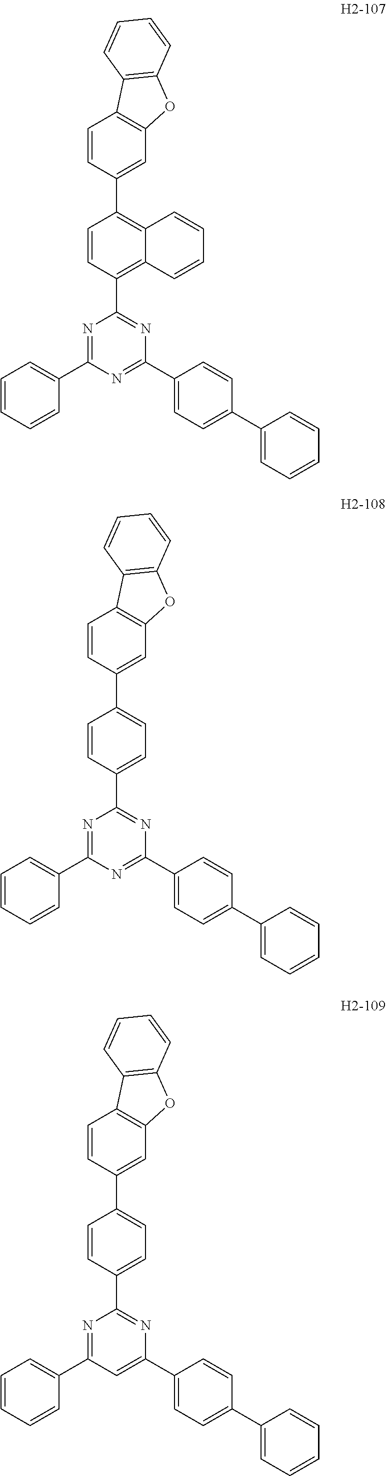 [SMILES: c1ccc(-c2ccc(-c3nc(-c4ccccc4)nc(-c4ccc(-c5ccc6c(c5)oc5ccccc56)c5ccccc45)n3)cc2)cc1 ; c1ccc(-c2ccc(-c3nc(-c4ccccc4)nc(-c4ccc(-c5ccc6c(c5)oc5ccccc56)cc4)n3)cc2)cc1 ; c1ccc(-c2ccc(-c3cc(-c4ccccc4)nc(-c4ccc(-c5ccc6c(c5)oc5ccccc56)cc4)n3)cc2)cc1]

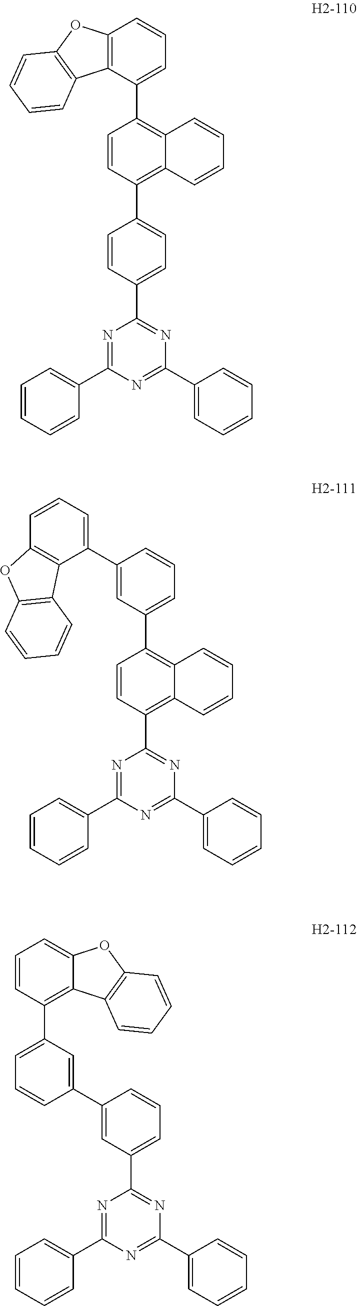 [SMILES: c1ccc(-c2nc(-c3ccccc3)nc(-c3ccc(-c4ccc(-c5cccc6oc7ccccc7c56)c5ccccc45)cc3)n2)cc1 ; c1ccc(-c2nc(-c3ccccc3)nc(-c3ccc(-c4cccc(-c5cccc6oc7ccccc7c56)c4)c4ccccc34)n2)cc1 ; c1ccc(-c2nc(-c3ccccc3)nc(-c3cccc(-c4cccc(-c5cccc6oc7ccccc7c56)c4)c3)n2)cc1]

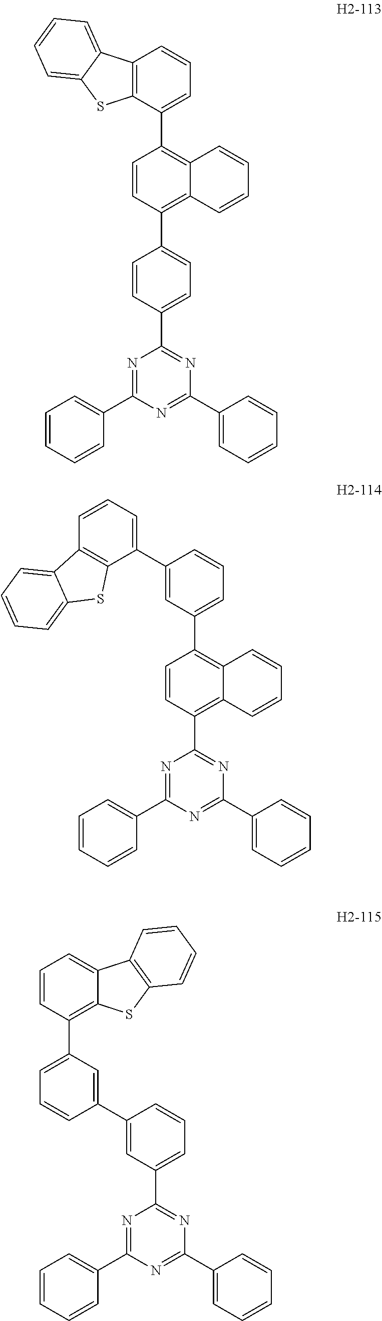 [SMILES: c1ccc(-c2nc(-c3ccccc3)nc(-c3ccc(-c4ccc(-c5cccc6c5sc5ccccc56)c5ccccc45)cc3)n2)cc1 ; c1ccc(-c2nc(-c3ccccc3)nc(-c3ccc(-c4cccc(-c5cccc6c5sc5ccccc56)c4)c4ccccc34)n2)cc1 ; c1ccc(-c2nc(-c3ccccc3)nc(-c3cccc(-c4cccc(-c5cccc6c5sc5ccccc56)c4)c3)n2)cc1]

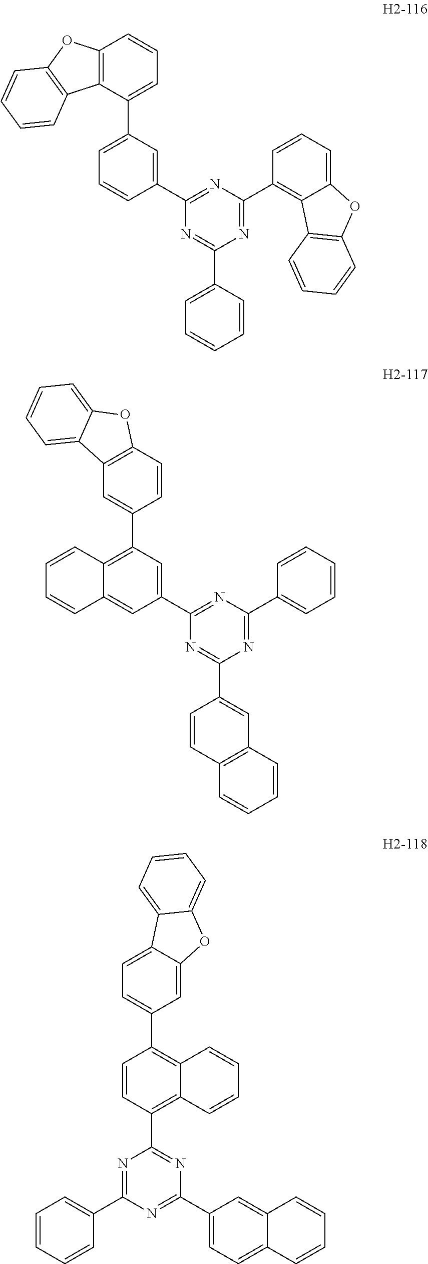 [SMILES: c1ccc(-c2nc(-c3cccc(-c4cccc5oc6ccccc6c45)c3)nc(-c3cccc4oc5ccccc5c34)n2)cc1 ; c1ccc(-c2nc(-c3ccc4ccccc4c3)nc(-c3cc(-c4ccc5oc6ccccc6c5c4)c4ccccc4c3)n2)cc1 ; c1ccc(-c2nc(-c3ccc4ccccc4c3)nc(-c3ccc(-c4ccc5c(c4)oc4ccccc45)c4ccccc34)n2)cc1]

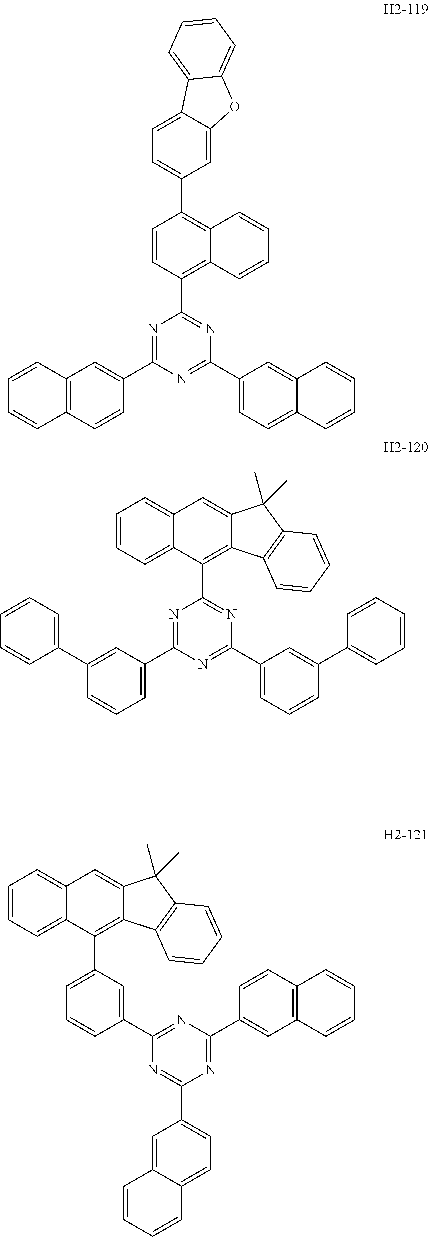

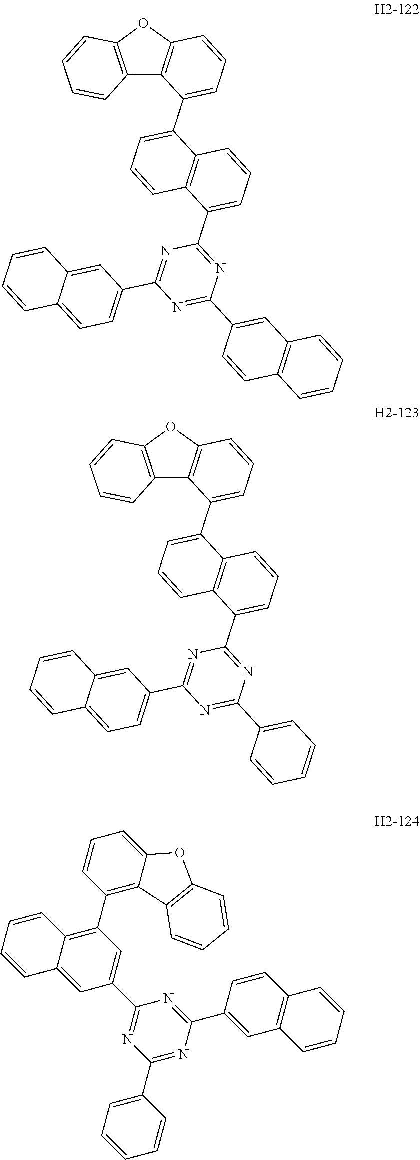 [SMILES: c1ccc2cc(-c3nc(-c4ccc5ccccc5c4)nc(-c4cccc5c(-c6cccc7oc8ccccc8c67)cccc45)n3)ccc2c1 ; c1ccc(-c2nc(-c3ccc4ccccc4c3)nc(-c3cccc4c(-c5cccc6oc7ccccc7c56)cccc34)n2)cc1 ; c1ccc(-c2nc(-c3ccc4ccccc4c3)nc(-c3cc(-c4cccc5oc6ccccc6c45)c4ccccc4c3)n2)cc1]

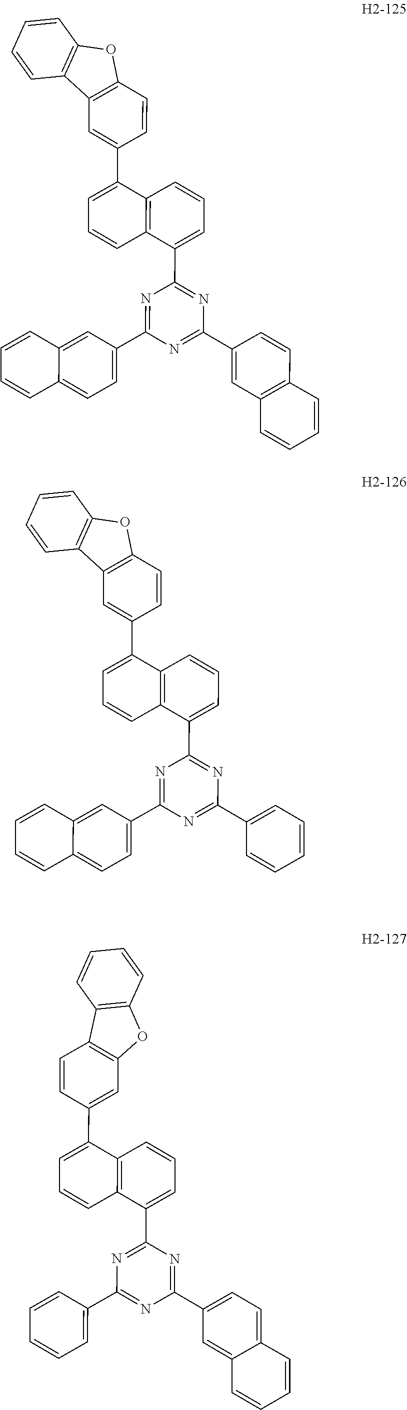 [SMILES: c1ccc2cc(-c3nc(-c4ccc5ccccc5c4)nc(-c4cccc5c(-c6ccc7oc8ccccc8c7c6)cccc45)n3)ccc2c1 ; c1ccc(-c2nc(-c3ccc4ccccc4c3)nc(-c3cccc4c(-c5ccc6oc7ccccc7c6c5)cccc34)n2)cc1 ; c1ccc(-c2nc(-c3ccc4ccccc4c3)nc(-c3cccc4c(-c5ccc6c(c5)oc5ccccc56)cccc34)n2)cc1]

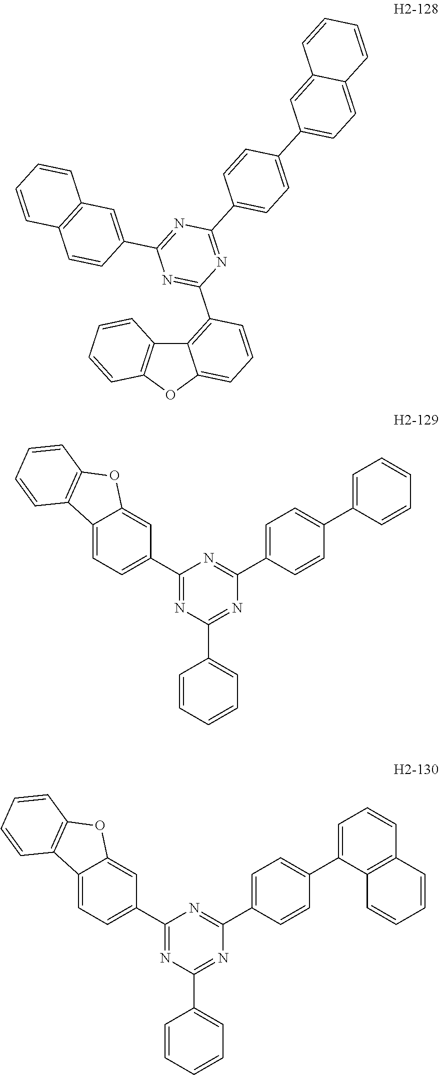 [SMILES: c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3nc(-c4ccccc4)nc(-c4ccc5c(c4)oc4ccccc45)n3)cc2)cc1 ; c1ccc(-c2nc(-c3ccc(-c4cccc5ccccc45)cc3)nc(-c3ccc4c(c3)oc3ccccc34)n2)cc1]

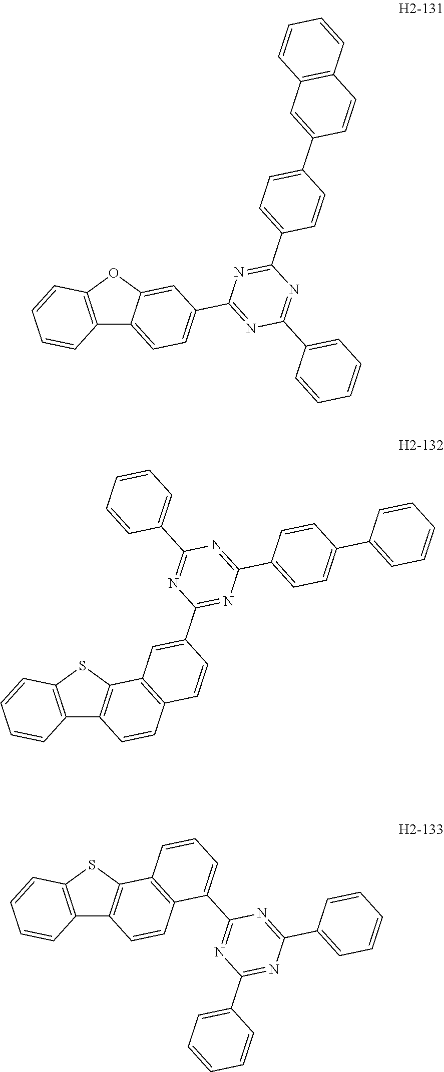 [SMILES: c1ccc(-c2nc(-c3ccc(-c4ccc5ccccc5c4)cc3)nc(-c3ccc4c(c3)oc3ccccc34)n2)cc1 ; c1ccc(-c2ccc(-c3nc(-c4ccccc4)nc(-c4ccc5ccc6c7ccccc7sc6c5c4)n3)cc2)cc1 ; c1ccc(-c2nc(-c3ccccc3)nc(-c3cccc4c3ccc3c5ccccc5sc43)n2)cc1]

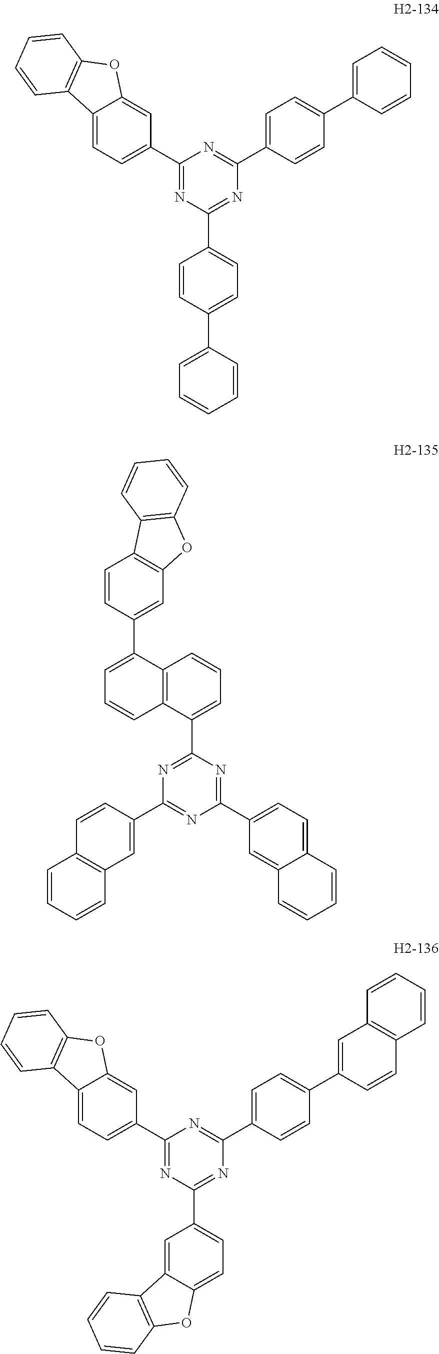 [SMILES: c1ccc(-c2ccc(-c3nc(-c4ccc(-c5ccccc5)cc4)nc(-c4ccc5c(c4)oc4ccccc45)n3)cc2)cc1 ; c1ccc2cc(-c3nc(-c4ccc5ccccc5c4)nc(-c4cccc5c(-c6ccc7c(c6)oc6ccccc67)cccc45)n3)ccc2c1 ; c1ccc2cc(-c3ccc(-c4nc(-c5ccc6c(c5)oc5ccccc56)nc(-c5ccc6oc7ccccc7c6c5)n4)cc3)ccc2c1]

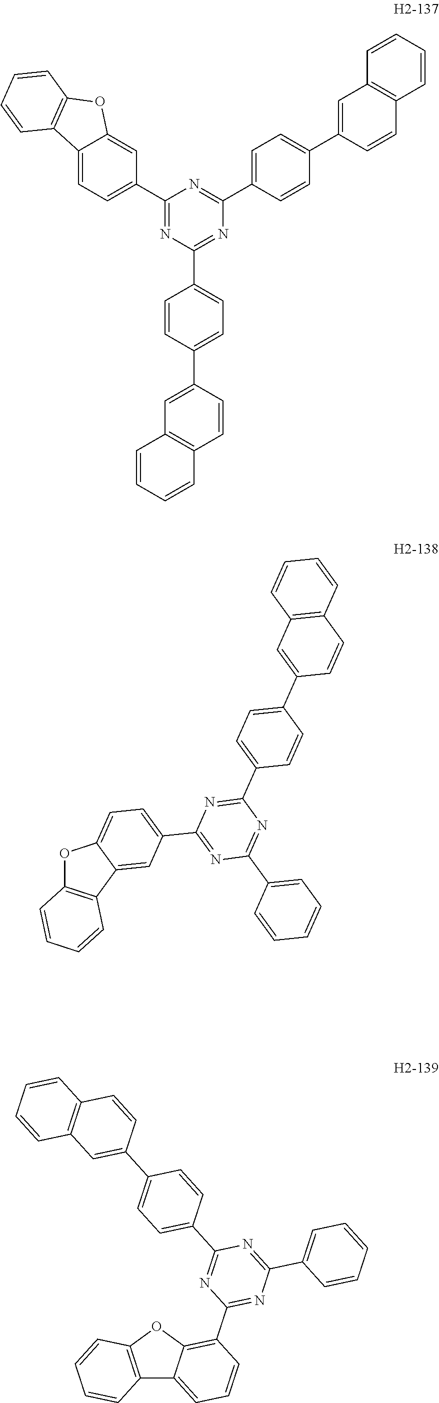 [SMILES: c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1 ; c1ccc(-c2nc(-c3ccc(-c4ccc5ccccc5c4)cc3)nc(-c3cccc4c3oc3ccccc34)n2)cc1]

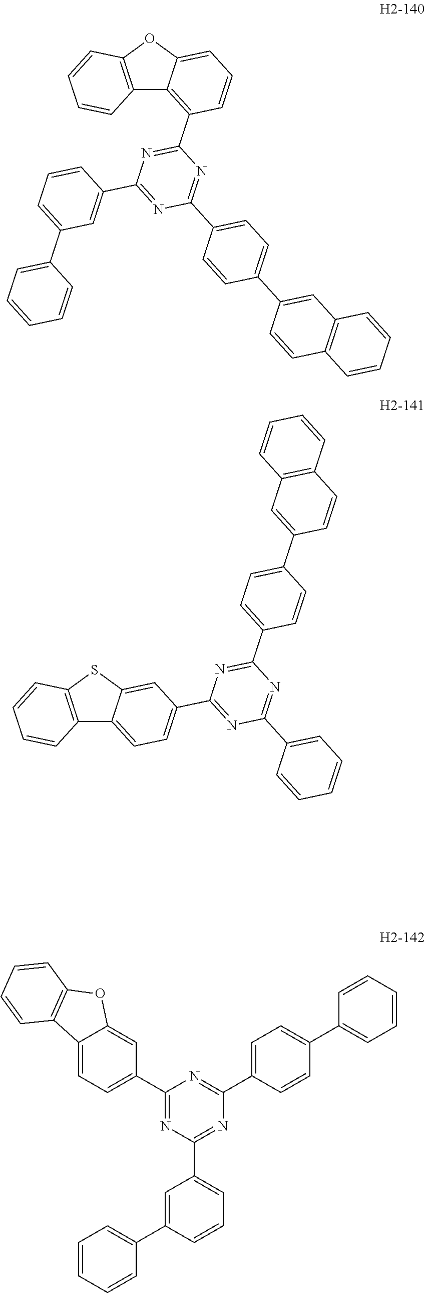 [SMILES: c1ccc(-c2cccc(-c3nc(-c4ccc(-c5ccc6ccccc6c5)cc4)nc(-c4cccc5oc6ccccc6c45)n3)c2)cc1 ; c1ccc(-c2nc(-c3ccc(-c4ccc5ccccc5c4)cc3)nc(-c3ccc4c(c3)sc3ccccc34)n2)cc1 ; c1ccc(-c2ccc(-c3nc(-c4cccc(-c5ccccc5)c4)nc(-c4ccc5c(c4)oc4ccccc45)n3)cc2)cc1]

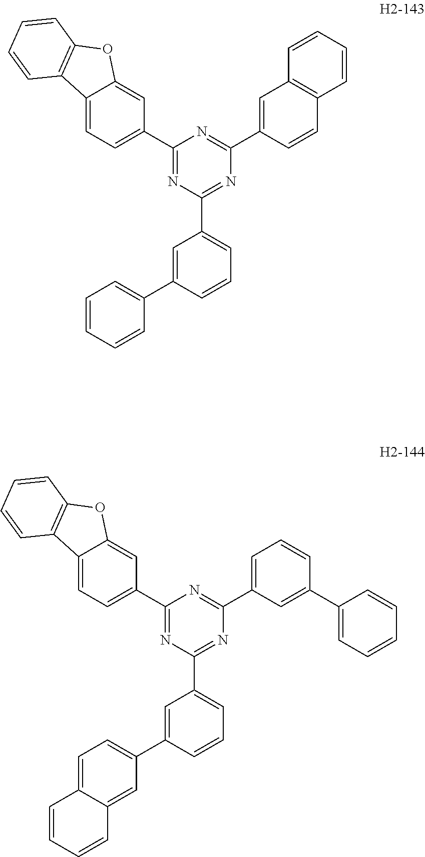 [SMILES: c1ccc(-c2cccc(-c3nc(-c4ccc5ccccc5c4)nc(-c4ccc5c(c4)oc4ccccc45)n3)c2)cc1 ; c1ccc(-c2cccc(-c3nc(-c4cccc(-c5ccc6ccccc6c5)c4)nc(-c4ccc5c(c4)oc4ccccc45)n3)c2)cc1]

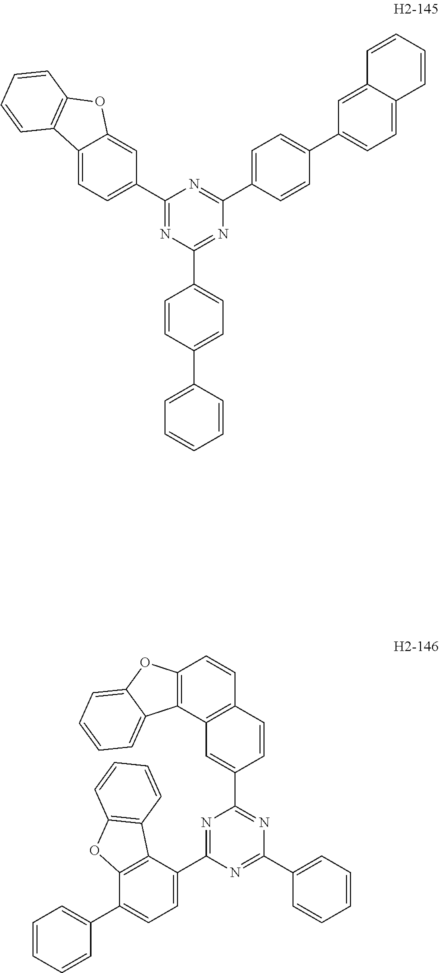 [SMILES: c1ccc(-c2ccc(-c3nc(-c4ccc(-c5ccc6ccccc6c5)cc4)nc(-c4ccc5c(c4)oc4ccccc45)n3)cc2)cc1 ; c1ccc(-c2nc(-c3ccc4ccc5oc6ccccc6c5c4c3)nc(-c3ccc(-c4ccccc4)c4oc5ccccc5c34)n2)cc1]

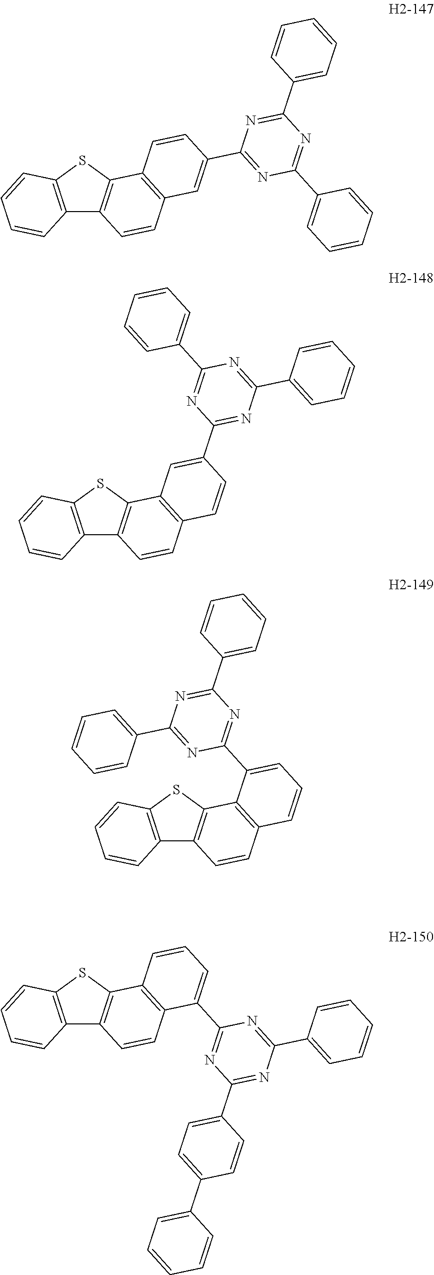 [SMILES: c1ccc(-c2nc(-c3ccccc3)nc(-c3ccc4c(ccc5c6ccccc6sc45)c3)n2)cc1 ; c1ccc(-c2nc(-c3ccccc3)nc(-c3ccc4ccc5c6ccccc6sc5c4c3)n2)cc1 ; c1ccc(-c2nc(-c3ccccc3)nc(-c3cccc4ccc5c6ccccc6sc5c34)n2)cc1 ; c1ccc(-c2ccc(-c3nc(-c4ccccc4)nc(-c4cccc5c4ccc4c6ccccc6sc54)n3)cc2)cc1]

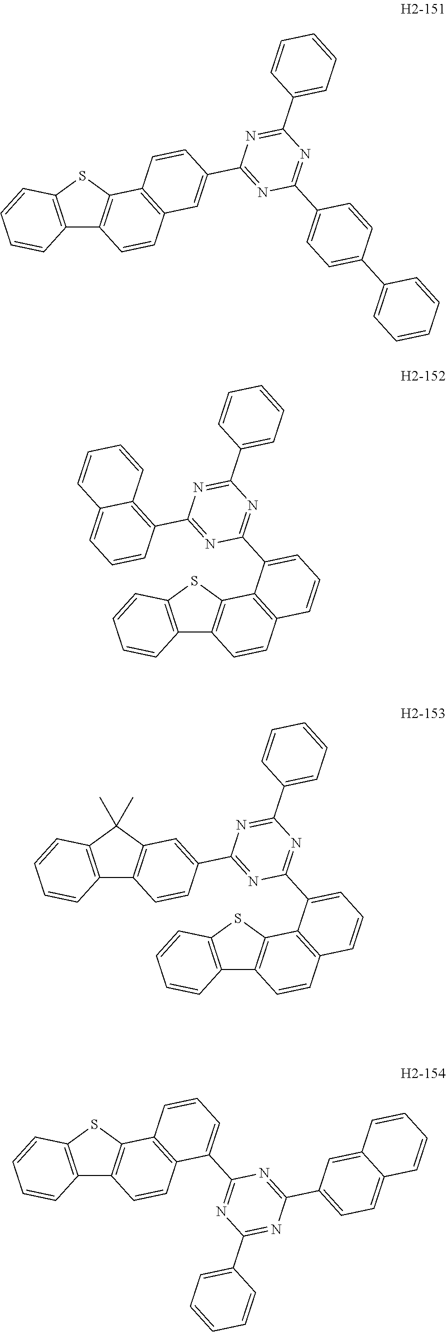 [SMILES: c1ccc(-c2ccc(-c3nc(-c4ccccc4)nc(-c4ccc5c(ccc6c7ccccc7sc56)c4)n3)cc2)cc1 ; c1ccc(-c2nc(-c3cccc4ccccc34)nc(-c3cccc4ccc5c6ccccc6sc5c34)n2)cc1 ; CC1(C)c2ccccc2-c2ccc(-c3nc(-c4ccccc4)nc(-c4cccc5ccc6c7ccccc7sc6c45)n3)cc21 ; c1ccc(-c2nc(-c3ccc4ccccc4c3)nc(-c3cccc4c3ccc3c5ccccc5sc43)n2)cc1]

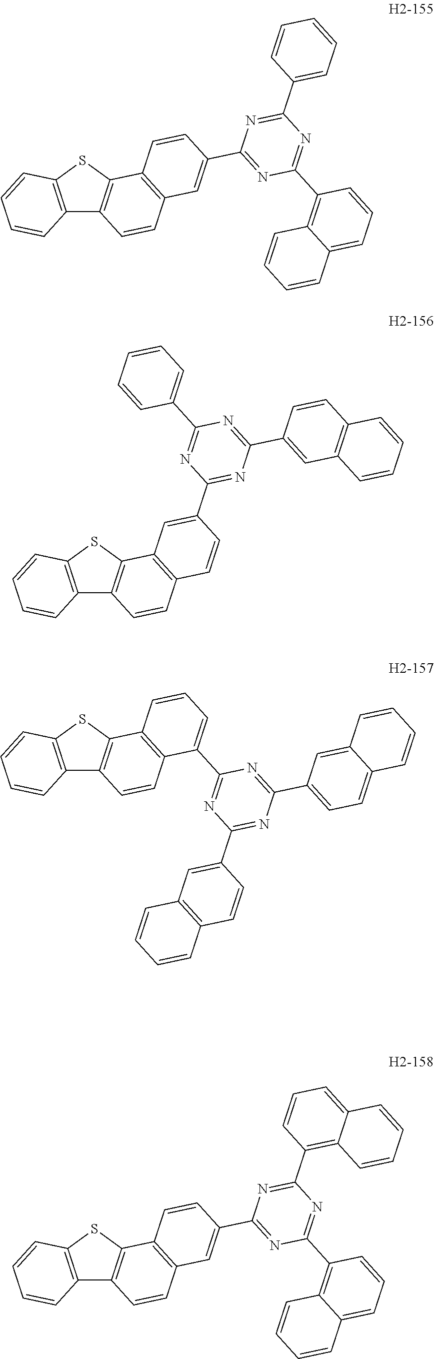 [SMILES: c1ccc(-c2nc(-c3ccc4c(ccc5c6ccccc6sc45)c3)nc(-c3cccc4ccccc34)n2)cc1 ; c1ccc(-c2nc(-c3ccc4ccccc4c3)nc(-c3ccc4ccc5c6ccccc6sc5c4c3)n2)cc1 ; c1ccc2cc(-c3nc(-c4ccc5ccccc5c4)nc(-c4cccc5c4ccc4c6ccccc6sc54)n3)ccc2c1 ; c1ccc2c(-c3nc(-c4ccc5c(ccc6c7ccccc7sc56)c4)nc(-c4cccc5ccccc45)n3)cccc2c1]

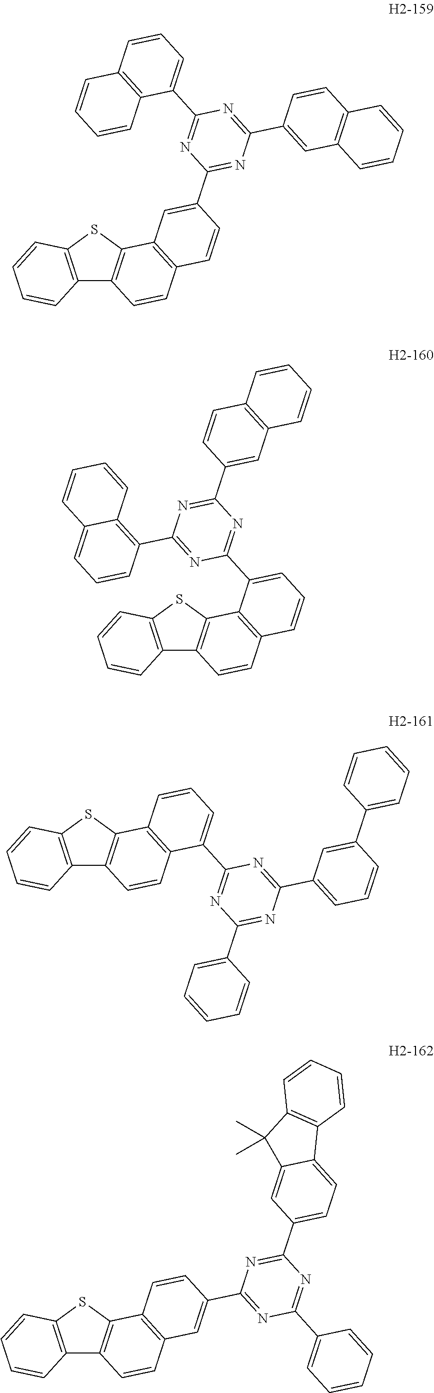 [SMILES: c1ccc2cc(-c3nc(-c4ccc5ccc6c7ccccc7sc6c5c4)nc(-c4cccc5ccccc45)n3)ccc2c1 ; c1ccc2cc(-c3nc(-c4cccc5ccccc45)nc(-c4cccc5ccc6c7ccccc7sc6c45)n3)ccc2c1 ; c1ccc(-c2cccc(-c3nc(-c4ccccc4)nc(-c4cccc5c4ccc4c6ccccc6sc54)n3)c2)cc1 ; CC1(C)c2ccccc2-c2ccc(-c3nc(-c4ccccc4)nc(-c4ccc5c(ccc6c7ccccc7sc56)c4)n3)cc21]

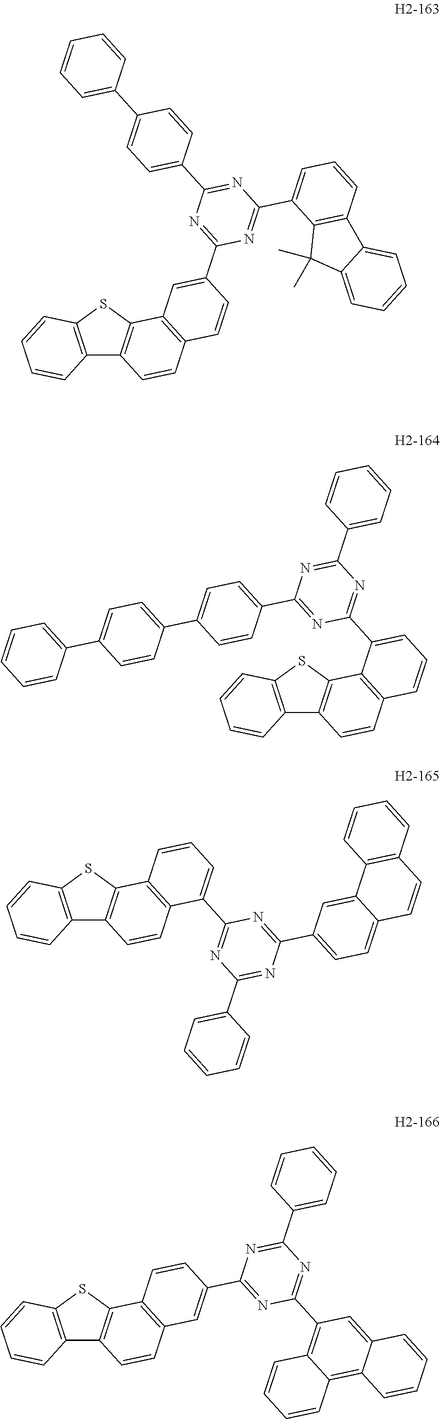 [SMILES: CC1(C)c2ccccc2-c2cccc(-c3nc(-c4ccc(-c5ccccc5)cc4)nc(-c4ccc5ccc6c7ccccc7sc6c5c4)n3)c21 ; c1ccc(-c2ccc(-c3ccc(-c4nc(-c5ccccc5)nc(-c5cccc6ccc7c8ccccc8sc7c56)n4)cc3)cc2)cc1 ; c1ccc(-c2nc(-c3ccc4ccc5ccccc5c4c3)nc(-c3cccc4c3ccc3c5ccccc5sc43)n2)cc1 ; c1ccc(-c2nc(-c3ccc4c(ccc5c6ccccc6sc45)c3)nc(-c3cc4ccccc4c4ccccc34)n2)cc1]

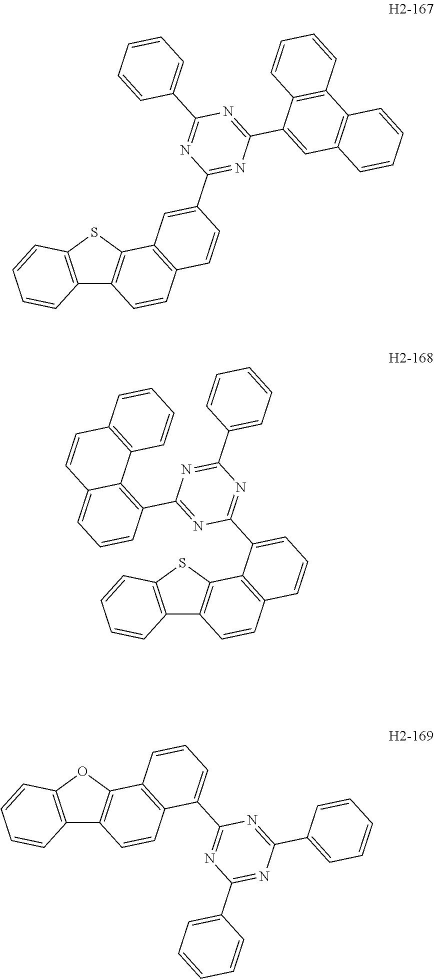 [SMILES: c1ccc(-c2nc(-c3ccc4ccc5c6ccccc6sc5c4c3)nc(-c3cc4ccccc4c4ccccc34)n2)cc1 ; c1ccc(-c2nc(-c3cccc4ccc5ccccc5c34)nc(-c3cccc4ccc5c6ccccc6sc5c34)n2)cc1 ; c1ccc(-c2nc(-c3ccccc3)nc(-c3cccc4c3ccc3c5ccccc5oc43)n2)cc1]

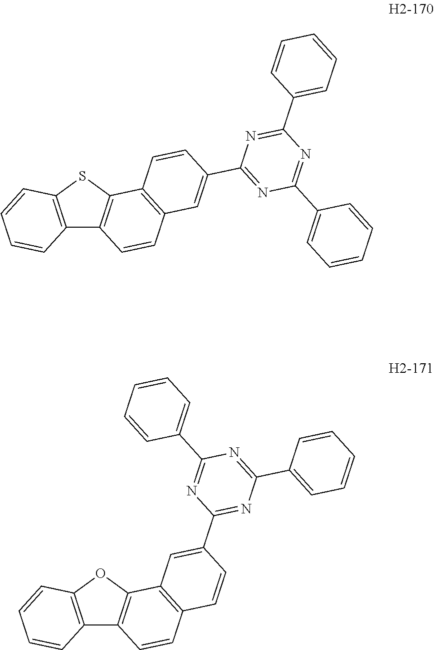 [SMILES: c1ccc(-c2nc(-c3ccccc3)nc(-c3ccc4c(ccc5c6ccccc6sc45)c3)n2)cc1 ; c1ccc(-c2nc(-c3ccccc3)nc(-c3ccc4ccc5c6ccccc6oc5c4c3)n2)cc1]

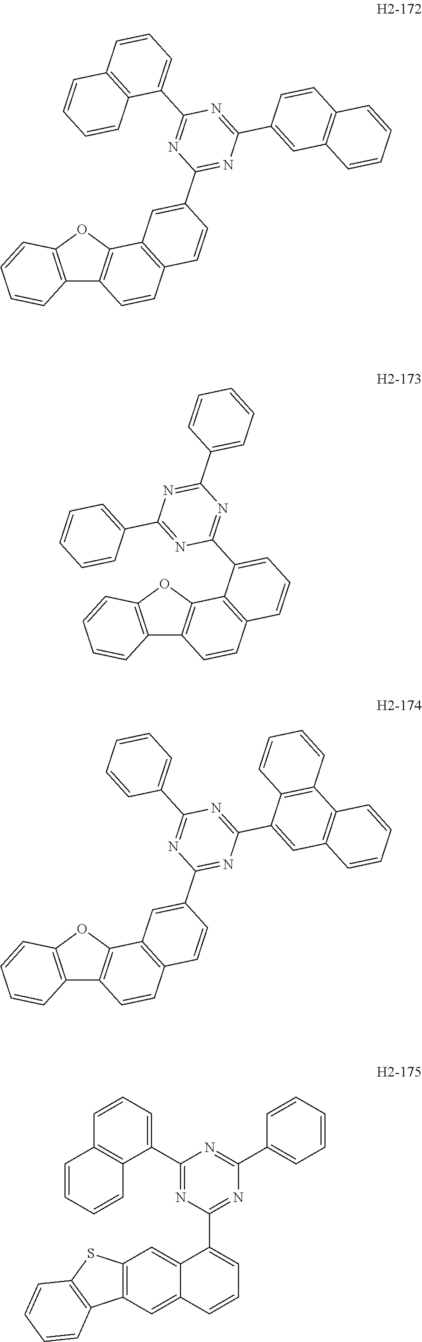 [SMILES: c1ccc2cc(-c3nc(-c4ccc5ccc6c7ccccc7oc6c5c4)nc(-c4cccc5ccccc45)n3)ccc2c1 ; c1ccc(-c2nc(-c3ccccc3)nc(-c3cccc4ccc5c6ccccc6oc5c34)n2)cc1 ; c1ccc(-c2nc(-c3ccc4ccc5c6ccccc6oc5c4c3)nc(-c3cc4ccccc4c4ccccc34)n2)cc1 ; c1ccc(-c2nc(-c3cccc4ccccc34)nc(-c3cccc4cc5c(cc34)sc3ccccc35)n2)cc1]

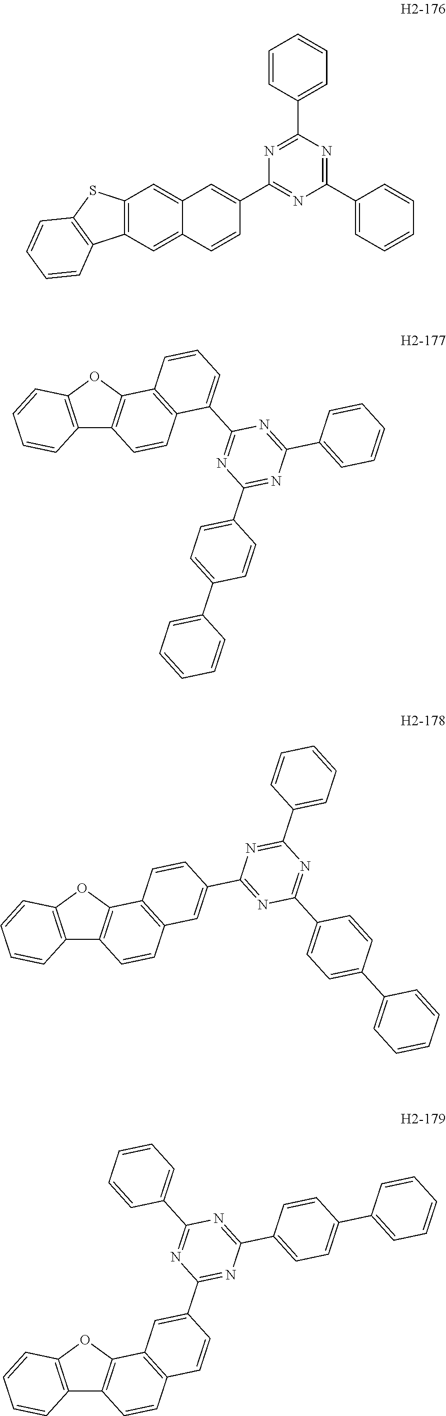 [SMILES: c1ccc(-c2nc(-c3ccccc3)nc(-c3ccc4cc5c(cc4c3)sc3ccccc35)n2)cc1 ; c1ccc(-c2ccc(-c3nc(-c4ccccc4)nc(-c4cccc5c4ccc4c6ccccc6oc54)n3)cc2)cc1 ; c1ccc(-c2ccc(-c3nc(-c4ccccc4)nc(-c4ccc5c(ccc6c7ccccc7oc56)c4)n3)cc2)cc1 ; c1ccc(-c2ccc(-c3nc(-c4ccccc4)nc(-c4ccc5ccc6c7ccccc7oc6c5c4)n3)cc2)cc1]

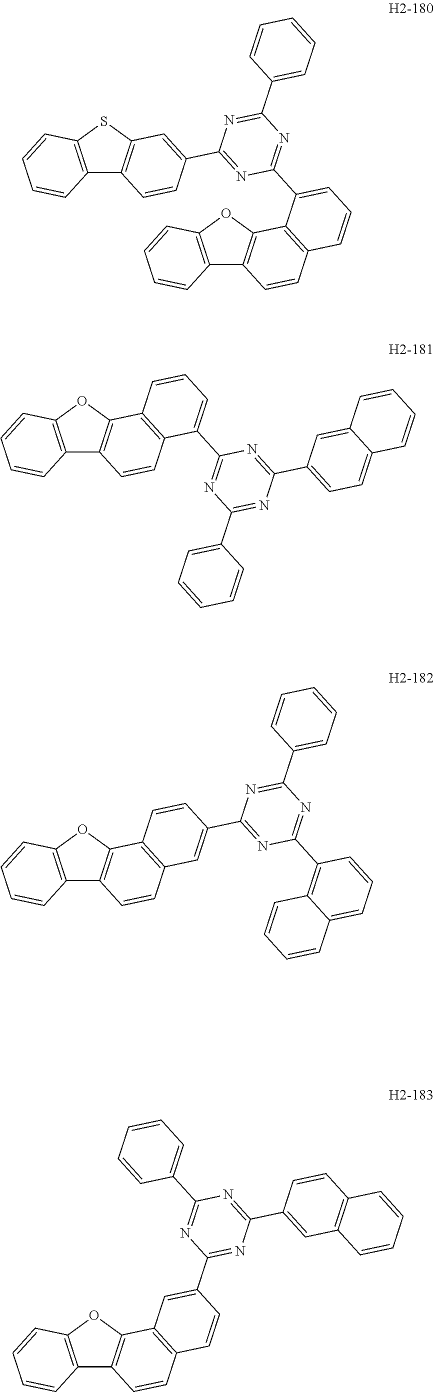 [SMILES: c1ccc(-c2nc(-c3ccc4c(c3)sc3ccccc34)nc(-c3cccc4ccc5c6ccccc6oc5c34)n2)cc1 ; c1ccc(-c2nc(-c3ccc4ccccc4c3)nc(-c3cccc4c3ccc3c5ccccc5oc43)n2)cc1 ; c1ccc(-c2nc(-c3ccc4c(ccc5c6ccccc6oc45)c3)nc(-c3cccc4ccccc34)n2)cc1 ; c1ccc(-c2nc(-c3ccc4ccccc4c3)nc(-c3ccc4ccc5c6ccccc6oc5c4c3)n2)cc1]

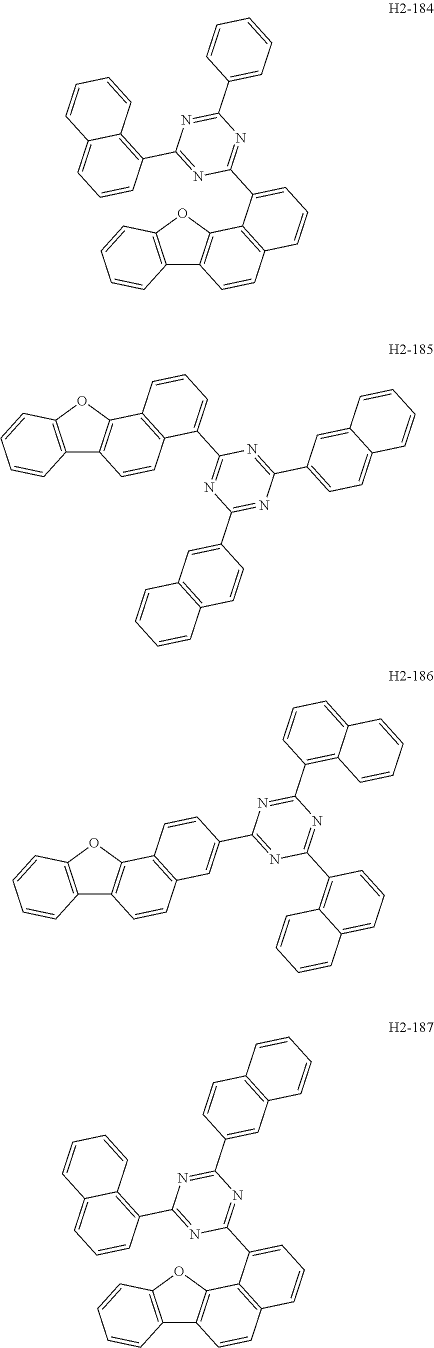 [SMILES: c1ccc(-c2nc(-c3cccc4ccccc34)nc(-c3cccc4ccc5c6ccccc6oc5c34)n2)cc1 ; c1ccc2cc(-c3nc(-c4ccc5ccccc5c4)nc(-c4cccc5c4ccc4c6ccccc6oc54)n3)ccc2c1 ; c1ccc2c(-c3nc(-c4ccc5c(ccc6c7ccccc7oc56)c4)nc(-c4cccc5ccccc45)n3)cccc2c1 ; c1ccc2cc(-c3nc(-c4cccc5ccccc45)nc(-c4cccc5ccc6c7ccccc7oc6c45)n3)ccc2c1]

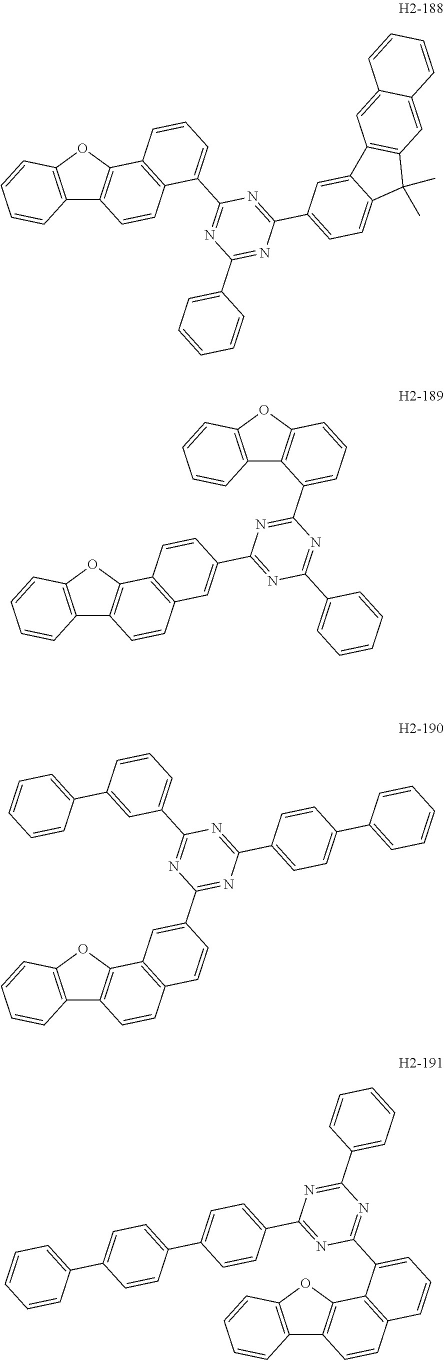 [SMILES: CC1(C)c2ccc(-c3nc(-c4ccccc4)nc(-c4cccc5c4ccc4c6ccccc6oc54)n3)cc2-c2cc3ccccc3cc21 ; c1ccc(-c2nc(-c3ccc4c(ccc5c6ccccc6oc45)c3)nc(-c3cccc4oc5ccccc5c34)n2)cc1 ; c1ccc(-c2ccc(-c3nc(-c4cccc(-c5ccccc5)c4)nc(-c4ccc5ccc6c7ccccc7oc6c5c4)n3)cc2)cc1 ; c1ccc(-c2ccc(-c3ccc(-c4nc(-c5ccccc5)nc(-c5cccc6ccc7c8ccccc8oc7c56)n4)cc3)cc2)cc1]

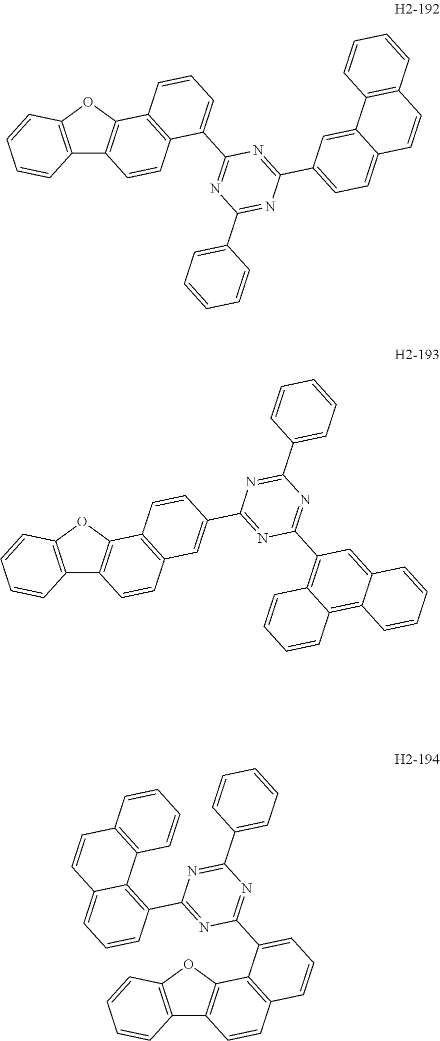 [SMILES: c1ccc(-c2nc(-c3ccc4ccc5ccccc5c4c3)nc(-c3cccc4c3ccc3c5ccccc5oc43)n2)cc1 ; c1ccc(-c2nc(-c3ccc4c(ccc5c6ccccc6oc45)c3)nc(-c3cc4ccccc4c4ccccc34)n2)cc1 ; c1ccc(-c2nc(-c3cccc4ccc5ccccc5c34)nc(-c3cccc4ccc5c6ccccc6oc5c34)n2)cc1]

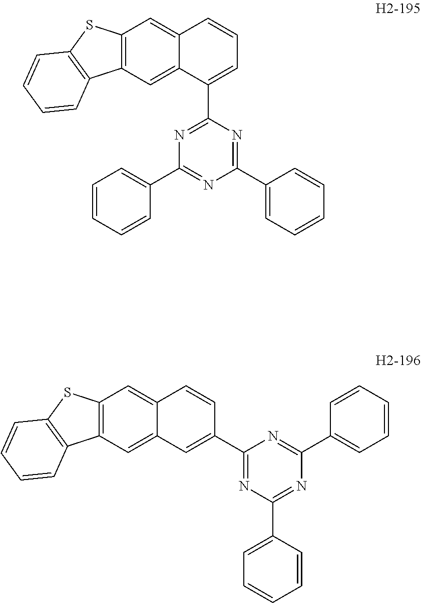 [SMILES: c1ccc(-c2nc(-c3ccccc3)nc(-c3cccc4cc5sc6ccccc6c5cc34)n2)cc1 ; c1ccc(-c2nc(-c3ccccc3)nc(-c3ccc4cc5sc6ccccc6c5cc4c3)n2)cc1]

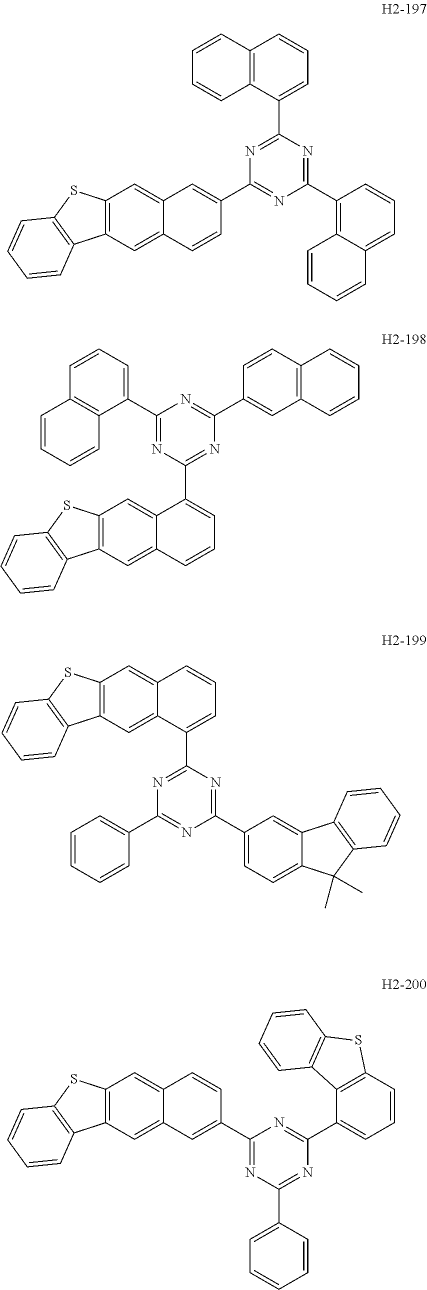 [SMILES: c1ccc2c(-c3nc(-c4ccc5cc6c(cc5c4)sc4ccccc46)nc(-c4cccc5ccccc45)n3)cccc2c1 ; c1ccc2cc(-c3nc(-c4cccc5ccccc45)nc(-c4cccc5cc6c(cc45)sc4ccccc46)n3)ccc2c1 ; CC1(C)c2ccccc2-c2cc(-c3nc(-c4ccccc4)nc(-c4cccc5cc6sc7ccccc7c6cc45)n3)ccc21 ; c1ccc(-c2nc(-c3ccc4cc5sc6ccccc6c5cc4c3)nc(-c3cccc4sc5ccccc5c34)n2)cc1]

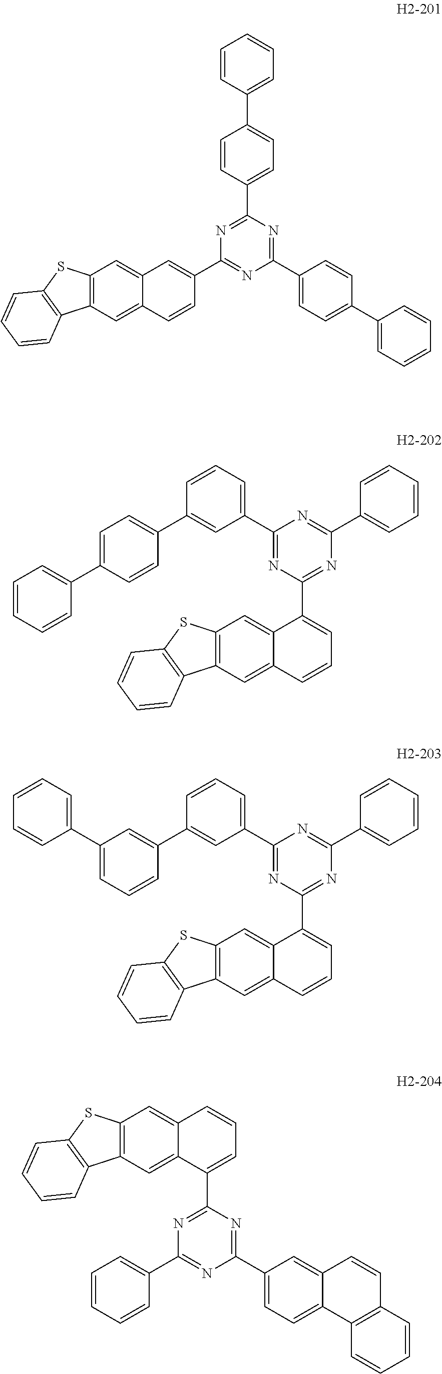 [SMILES: c1ccc(-c2ccc(-c3nc(-c4ccc(-c5ccccc5)cc4)nc(-c4ccc5cc6c(cc5c4)sc4ccccc46)n3)cc2)cc1 ; c1ccc(-c2ccc(-c3cccc(-c4nc(-c5ccccc5)nc(-c5cccc6cc7c(cc56)sc5ccccc57)n4)c3)cc2)cc1 ; c1ccc(-c2cccc(-c3cccc(-c4nc(-c5ccccc5)nc(-c5cccc6cc7c(cc56)sc5ccccc57)n4)c3)c2)cc1 ; c1ccc(-c2nc(-c3ccc4c(ccc5ccccc54)c3)nc(-c3cccc4cc5sc6ccccc6c5cc34)n2)cc1]

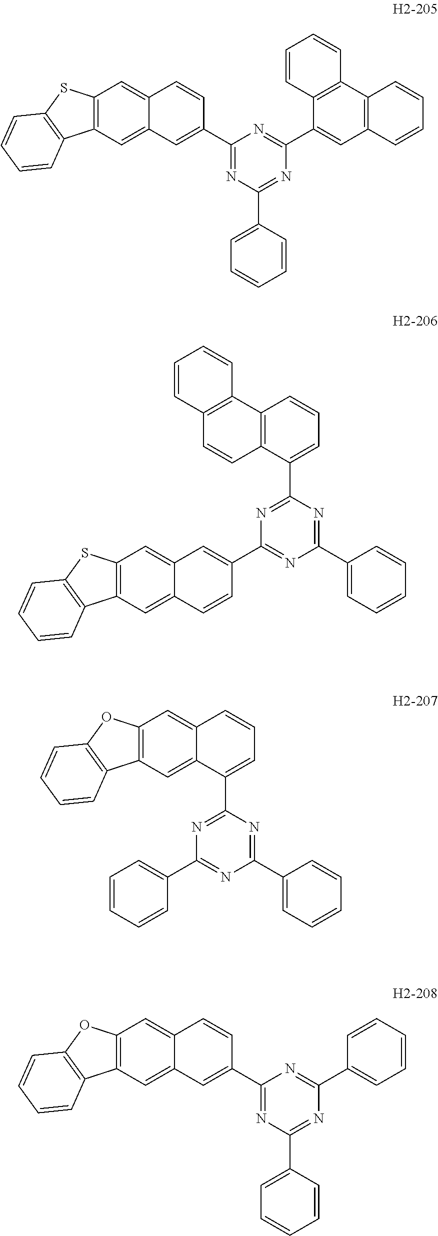 [SMILES: c1ccc(-c2nc(-c3ccc4cc5sc6ccccc6c5cc4c3)nc(-c3cc4ccccc4c4ccccc34)n2)cc1 ; c1ccc(-c2nc(-c3ccc4cc5c(cc4c3)sc3ccccc35)nc(-c3cccc4c3ccc3ccccc34)n2)cc1 ; c1ccc(-c2nc(-c3ccccc3)nc(-c3cccc4cc5oc6ccccc6c5cc34)n2)cc1 ; c1ccc(-c2nc(-c3ccccc3)nc(-c3ccc4cc5oc6ccccc6c5cc4c3)n2)cc1]

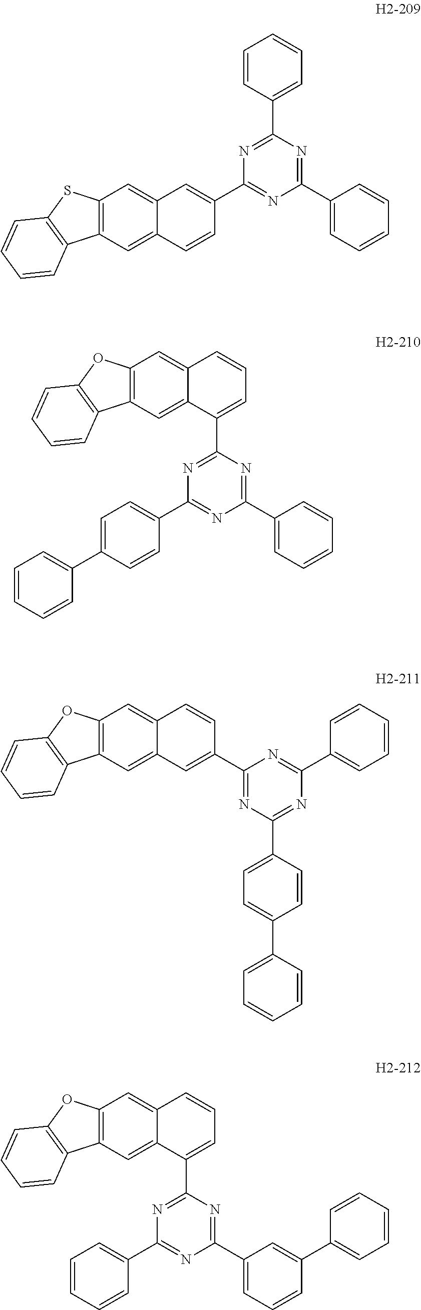 [SMILES: c1ccc(-c2nc(-c3ccccc3)nc(-c3ccc4cc5c(cc4c3)sc3ccccc35)n2)cc1 ; c1ccc(-c2ccc(-c3nc(-c4ccccc4)nc(-c4cccc5cc6oc7ccccc7c6cc45)n3)cc2)cc1 ; c1ccc(-c2ccc(-c3nc(-c4ccccc4)nc(-c4ccc5cc6oc7ccccc7c6cc5c4)n3)cc2)cc1 ; c1ccc(-c2cccc(-c3nc(-c4ccccc4)nc(-c4cccc5cc6oc7ccccc7c6cc45)n3)c2)cc1]

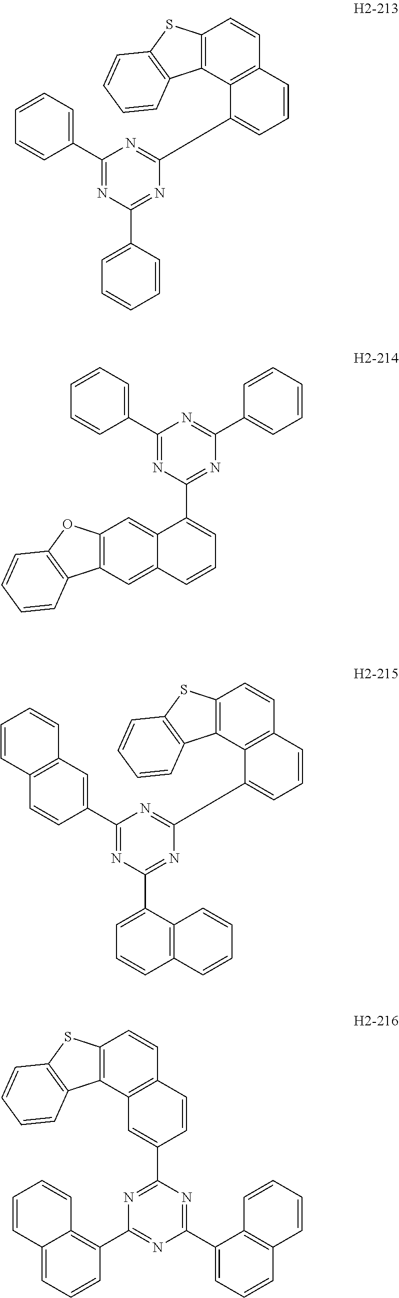 [SMILES: c1ccc(-c2nc(-c3ccccc3)nc(-c3cccc4ccc5sc6ccccc6c5c34)n2)cc1 ; c1ccc(-c2nc(-c3ccccc3)nc(-c3cccc4cc5c(cc34)oc3ccccc35)n2)cc1 ; c1ccc2cc(-c3nc(-c4cccc5ccccc45)nc(-c4cccc5ccc6sc7ccccc7c6c45)n3)ccc2c1 ; c1ccc2c(-c3nc(-c4ccc5ccc6sc7ccccc7c6c5c4)nc(-c4cccc5ccccc45)n3)cccc2c1]

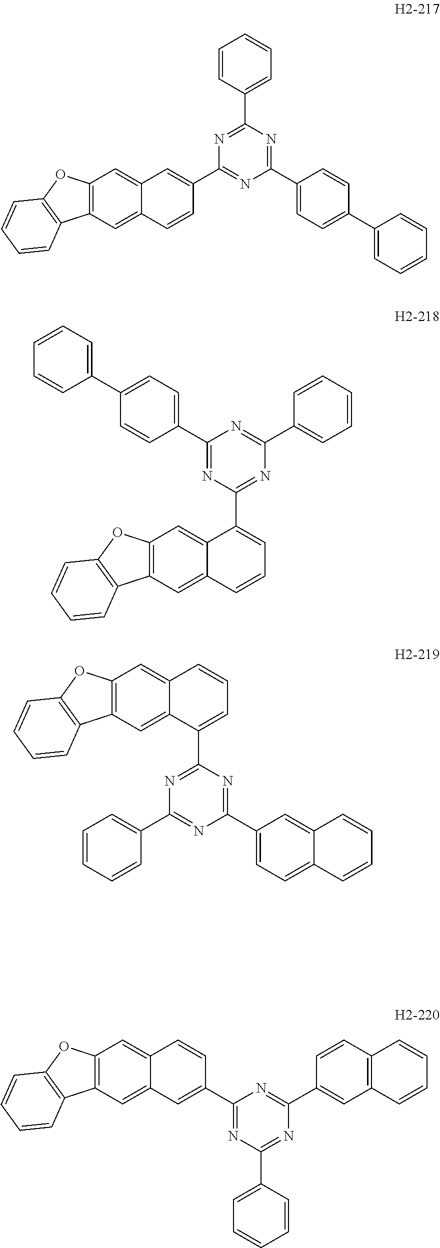 [SMILES: c1ccc(-c2ccc(-c3nc(-c4ccccc4)nc(-c4ccc5cc6c(cc5c4)oc4ccccc46)n3)cc2)cc1 ; c1ccc(-c2ccc(-c3nc(-c4ccccc4)nc(-c4cccc5cc6c(cc45)oc4ccccc46)n3)cc2)cc1 ; c1ccc(-c2nc(-c3ccc4ccccc4c3)nc(-c3cccc4cc5oc6ccccc6c5cc34)n2)cc1 ; c1ccc(-c2nc(-c3ccc4ccccc4c3)nc(-c3ccc4cc5oc6ccccc6c5cc4c3)n2)cc1]

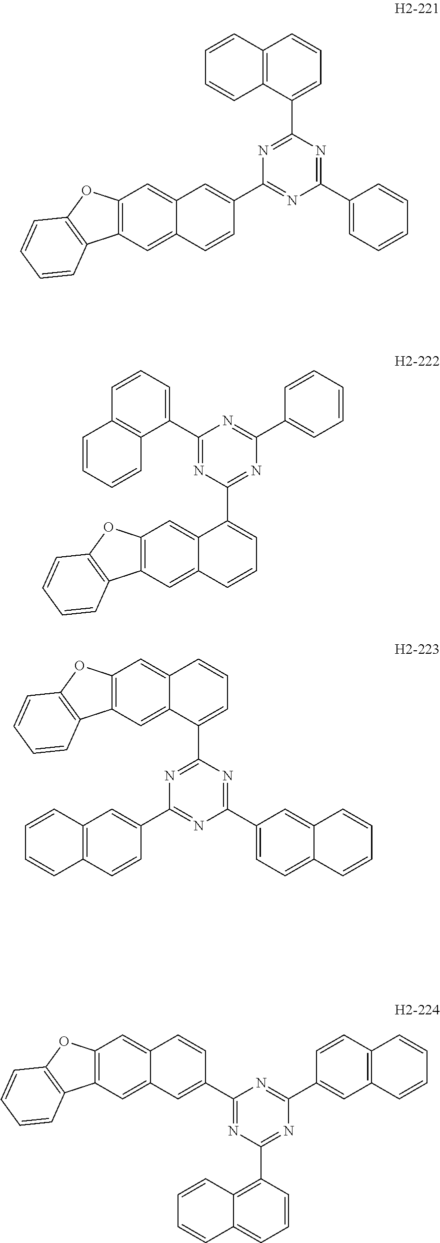 [SMILES: c1ccc(-c2nc(-c3ccc4cc5c(cc4c3)oc3ccccc35)nc(-c3cccc4ccccc34)n2)cc1 ; c1ccc(-c2nc(-c3cccc4ccccc34)nc(-c3cccc4cc5c(cc34)oc3ccccc35)n2)cc1 ; c1ccc2cc(-c3nc(-c4ccc5ccccc5c4)nc(-c4cccc5cc6oc7ccccc7c6cc45)n3)ccc2c1 ; c1ccc2cc(-c3nc(-c4ccc5cc6oc7ccccc7c6cc5c4)nc(-c4cccc5ccccc45)n3)ccc2c1]

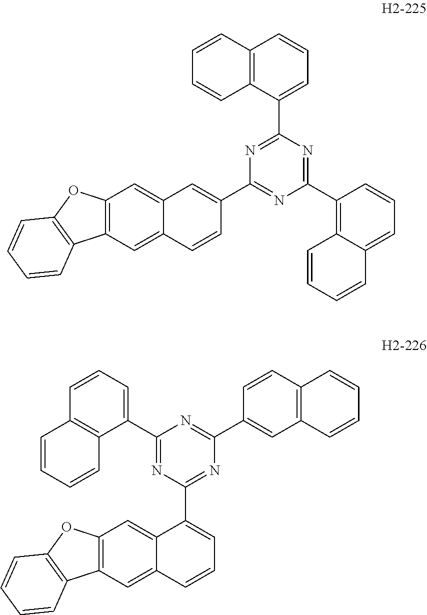 [SMILES: c1ccc2c(-c3nc(-c4ccc5cc6c(cc5c4)oc4ccccc46)nc(-c4cccc5ccccc45)n3)cccc2c1 ; c1ccc2cc(-c3nc(-c4cccc5ccccc45)nc(-c4cccc5cc6c(cc45)oc4ccccc46)n3)ccc2c1]

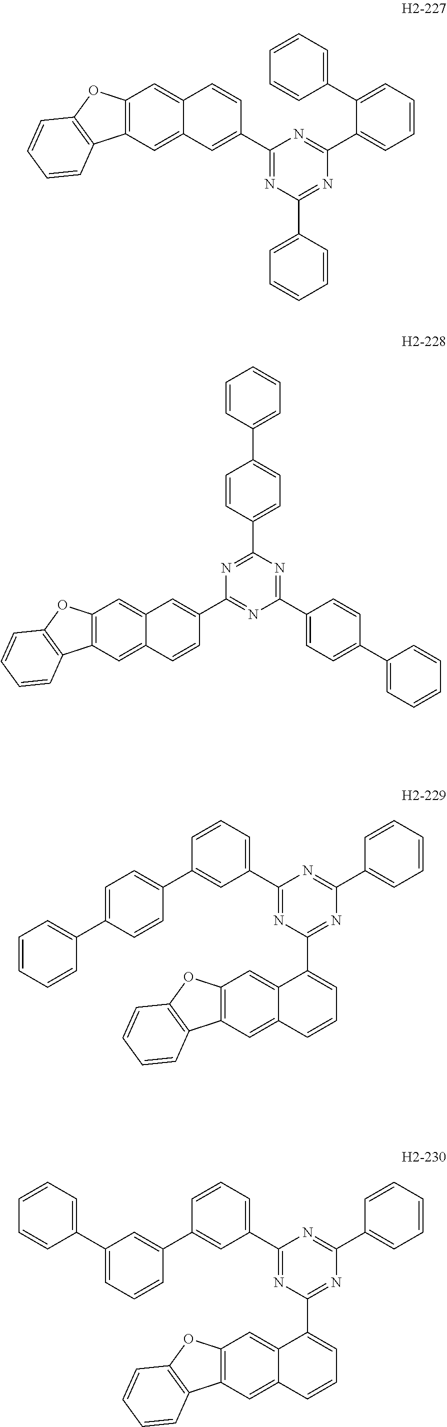 [SMILES: c1ccc(-c2nc(-c3ccc4cc5oc6ccccc6c5cc4c3)nc(-c3ccccc3-c3ccccc3)n2)cc1 ; c1ccc(-c2ccc(-c3nc(-c4ccc(-c5ccccc5)cc4)nc(-c4ccc5cc6c(cc5c4)oc4ccccc46)n3)cc2)cc1 ; c1ccc(-c2ccc(-c3cccc(-c4nc(-c5ccccc5)nc(-c5cccc6cc7c(cc56)oc5ccccc57)n4)c3)cc2)cc1 ; c1ccc(-c2cccc(-c3cccc(-c4nc(-c5ccccc5)nc(-c5cccc6cc7c(cc56)oc5ccccc57)n4)c3)c2)cc1]

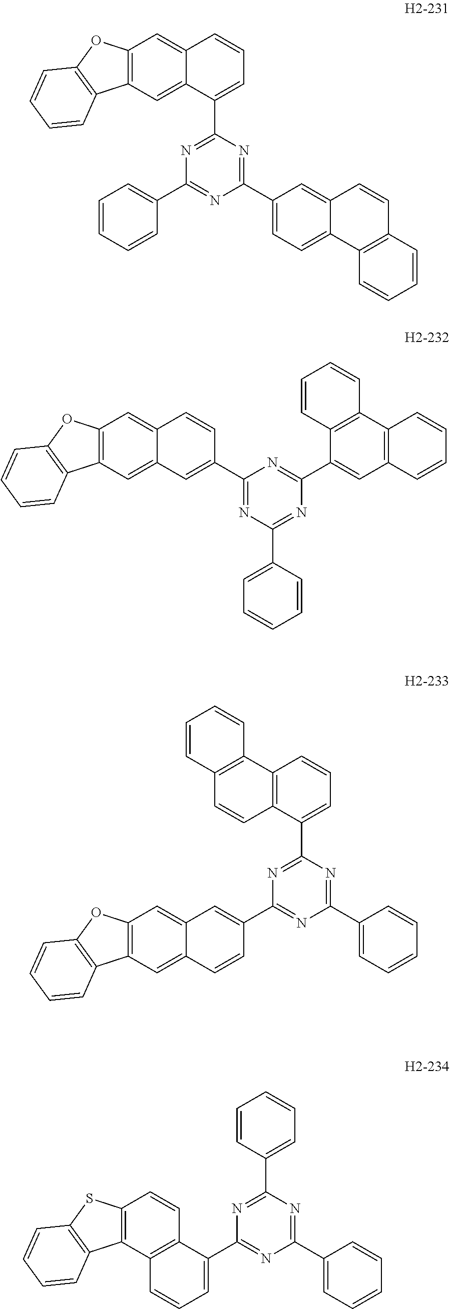 [SMILES: c1ccc(-c2nc(-c3ccc4c(ccc5ccccc54)c3)nc(-c3cccc4cc5oc6ccccc6c5cc34)n2)cc1 ; c1ccc(-c2nc(-c3ccc4cc5oc6ccccc6c5cc4c3)nc(-c3cc4ccccc4c4ccccc34)n2)cc1 ; c1ccc(-c2nc(-c3ccc4cc5c(cc4c3)oc3ccccc35)nc(-c3cccc4c3ccc3ccccc34)n2)cc1 ; c1ccc(-c2nc(-c3ccccc3)nc(-c3cccc4c3ccc3sc5ccccc5c34)n2)cc1]

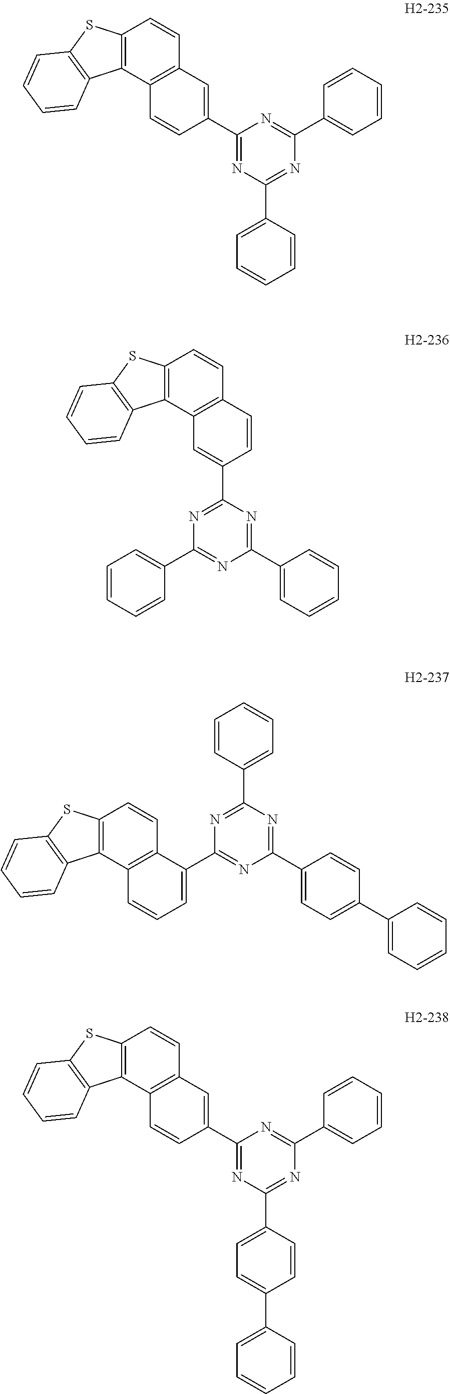 [SMILES: c1ccc(-c2nc(-c3ccccc3)nc(-c3ccc4c(ccc5sc6ccccc6c54)c3)n2)cc1 ; c1ccc(-c2nc(-c3ccccc3)nc(-c3ccc4ccc5sc6ccccc6c5c4c3)n2)cc1 ; c1ccc(-c2ccc(-c3nc(-c4ccccc4)nc(-c4cccc5c4ccc4sc6ccccc6c45)n3)cc2)cc1 ; c1ccc(-c2ccc(-c3nc(-c4ccccc4)nc(-c4ccc5c(ccc6sc7ccccc7c65)c4)n3)cc2)cc1]

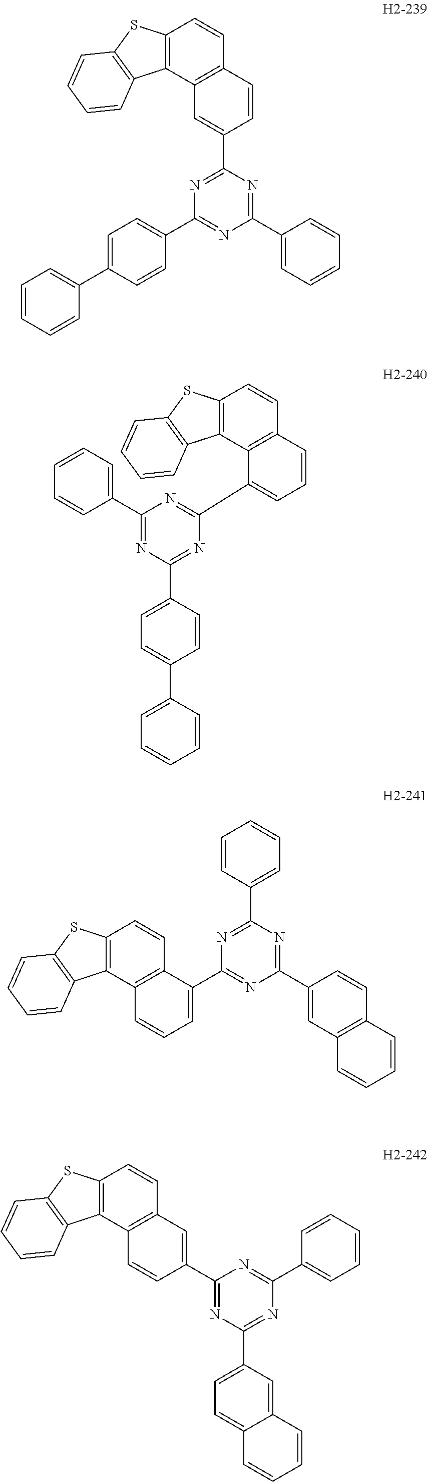 [SMILES: c1ccc(-c2ccc(-c3nc(-c4ccccc4)nc(-c4ccc5ccc6sc7ccccc7c6c5c4)n3)cc2)cc1 ; c1ccc(-c2ccc(-c3nc(-c4ccccc4)nc(-c4cccc5ccc6sc7ccccc7c6c45)n3)cc2)cc1 ; c1ccc(-c2nc(-c3ccc4ccccc4c3)nc(-c3cccc4c3ccc3sc5ccccc5c34)n2)cc1 ; c1ccc(-c2nc(-c3ccc4ccccc4c3)nc(-c3ccc4c(ccc5sc6ccccc6c54)c3)n2)cc1]

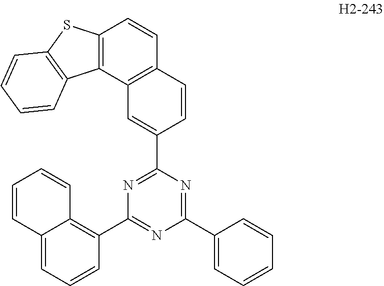 [SMILES: c1ccc(-c2nc(-c3ccc4ccc5sc6ccccc6c5c4c3)nc(-c3cccc4ccccc34)n2)cc1]

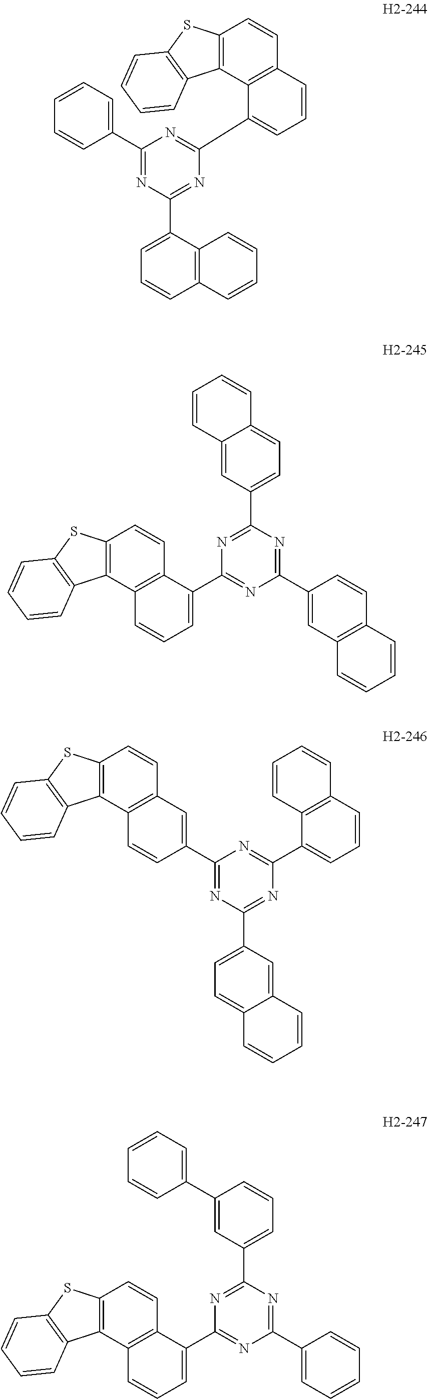 [SMILES: c1ccc(-c2nc(-c3cccc4ccccc34)nc(-c3cccc4ccc5sc6ccccc6c5c34)n2)cc1 ; c1ccc2cc(-c3nc(-c4ccc5ccccc5c4)nc(-c4cccc5c4ccc4sc6ccccc6c45)n3)ccc2c1 ; c1ccc2cc(-c3nc(-c4ccc5c(ccc6sc7ccccc7c65)c4)nc(-c4cccc5ccccc45)n3)ccc2c1 ; c1ccc(-c2cccc(-c3nc(-c4ccccc4)nc(-c4cccc5c4ccc4sc6ccccc6c45)n3)c2)cc1]

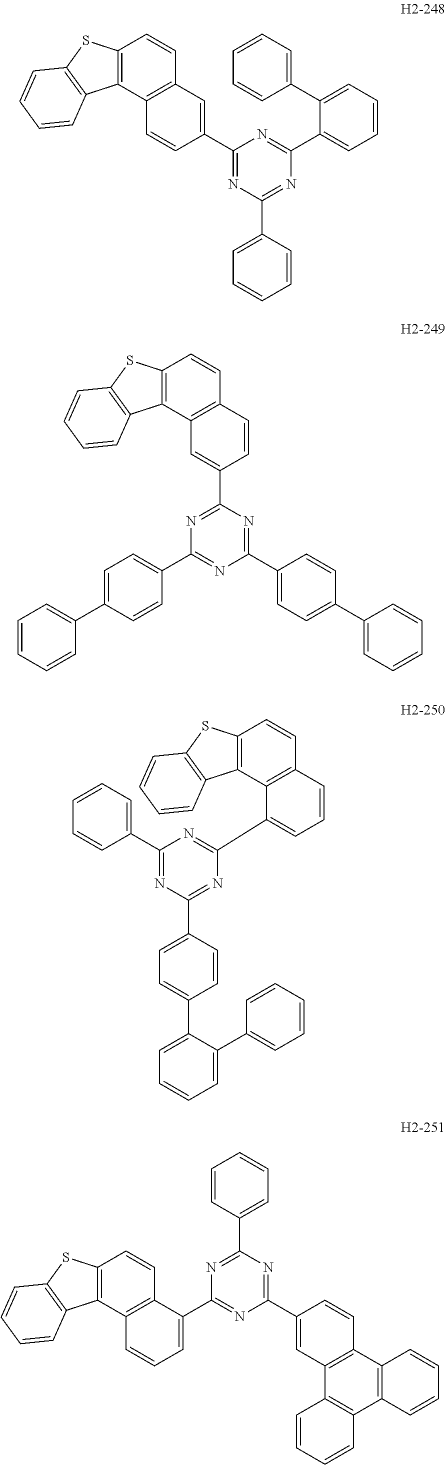 [SMILES: c1ccc(-c2nc(-c3ccc4c(ccc5sc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6sc7ccccc7c6c5c4)n3)cc2)cc1 ; c1ccc(-c2nc(-c3ccc(-c4ccccc4-c4ccccc4)cc3)nc(-c3cccc4ccc5sc6ccccc6c5c34)n2)cc1 ; c1ccc(-c2nc(-c3ccc4c5ccccc5c5ccccc5c4c3)nc(-c3cccc4c3ccc3sc5ccccc5c34)n2)cc1]

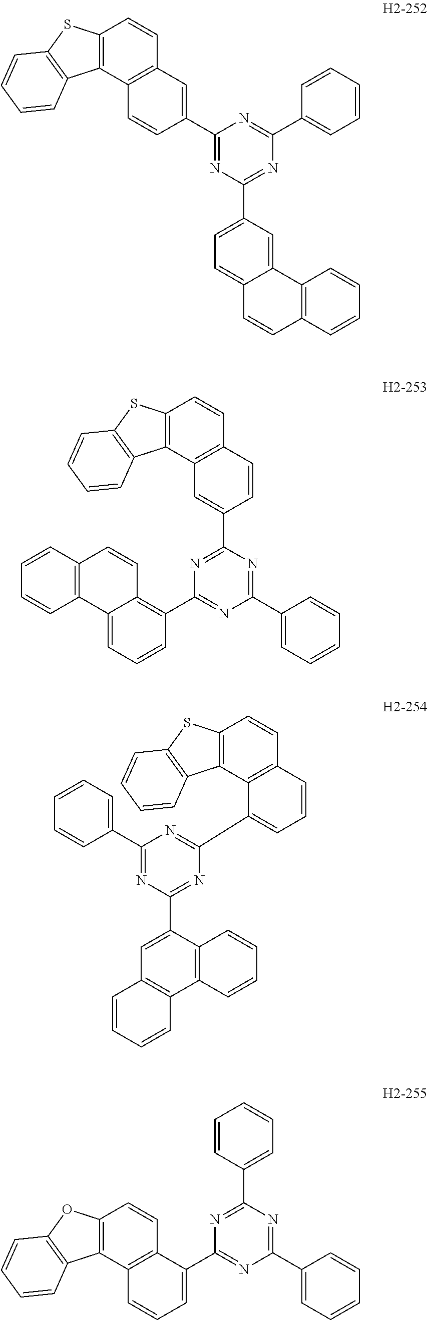 [SMILES: c1ccc(-c2nc(-c3ccc4c(ccc5sc6ccccc6c54)c3)nc(-c3ccc4ccc5ccccc5c4c3)n2)cc1 ; c1ccc(-c2nc(-c3ccc4ccc5sc6ccccc6c5c4c3)nc(-c3cccc4c3ccc3ccccc34)n2)cc1 ; c1ccc(-c2nc(-c3cc4ccccc4c4ccccc34)nc(-c3cccc4ccc5sc6ccccc6c5c34)n2)cc1 ; c1ccc(-c2nc(-c3ccccc3)nc(-c3cccc4c3ccc3oc5ccccc5c34)n2)cc1]

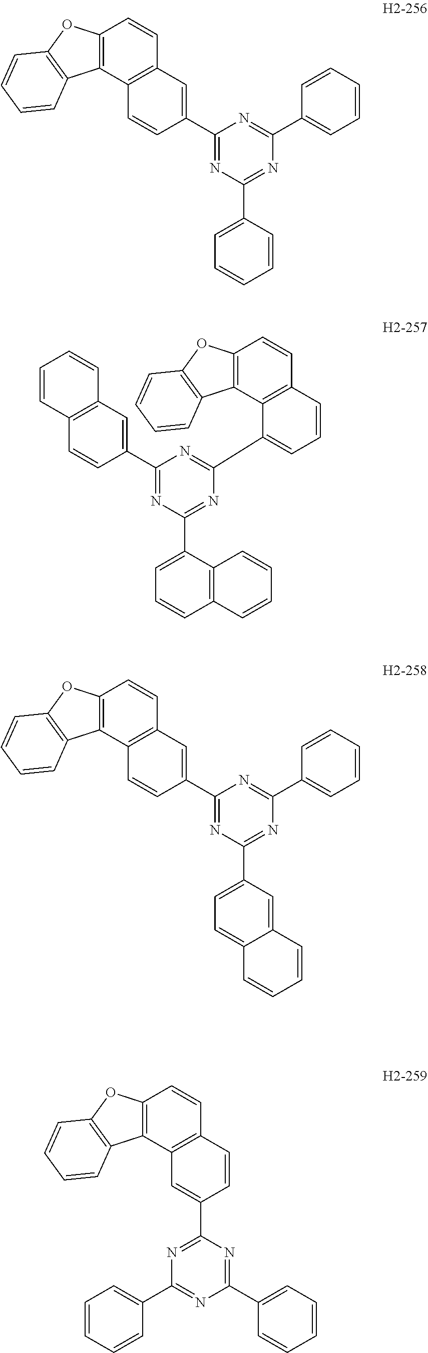 [SMILES: c1ccc(-c2nc(-c3ccccc3)nc(-c3ccc4c(ccc5oc6ccccc6c54)c3)n2)cc1 ; c1ccc2cc(-c3nc(-c4cccc5ccccc45)nc(-c4cccc5ccc6oc7ccccc7c6c45)n3)ccc2c1 ; c1ccc(-c2nc(-c3ccc4ccccc4c3)nc(-c3ccc4c(ccc5oc6ccccc6c54)c3)n2)cc1 ; c1ccc(-c2nc(-c3ccccc3)nc(-c3ccc4ccc5oc6ccccc6c5c4c3)n2)cc1]

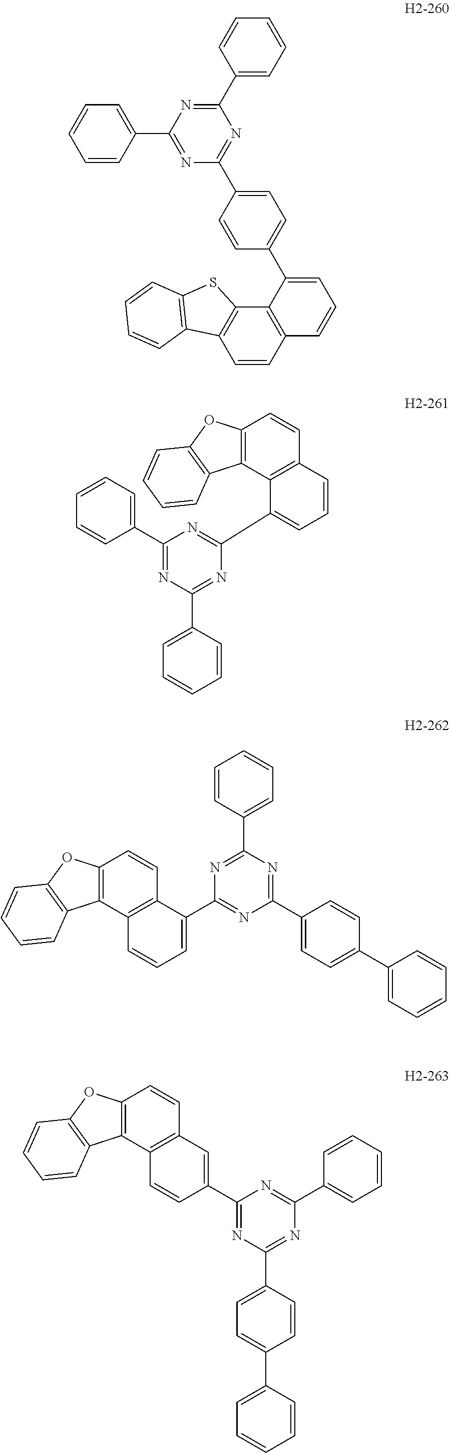 [SMILES: c1ccc(-c2nc(-c3ccccc3)nc(-c3ccc(-c4cccc5ccc6c7ccccc7sc6c45)cc3)n2)cc1 ; c1ccc(-c2nc(-c3ccccc3)nc(-c3cccc4ccc5oc6ccccc6c5c34)n2)cc1 ; c1ccc(-c2ccc(-c3nc(-c4ccccc4)nc(-c4cccc5c4ccc4oc6ccccc6c45)n3)cc2)cc1 ; c1ccc(-c2ccc(-c3nc(-c4ccccc4)nc(-c4ccc5c(ccc6oc7ccccc7c65)c4)n3)cc2)cc1]

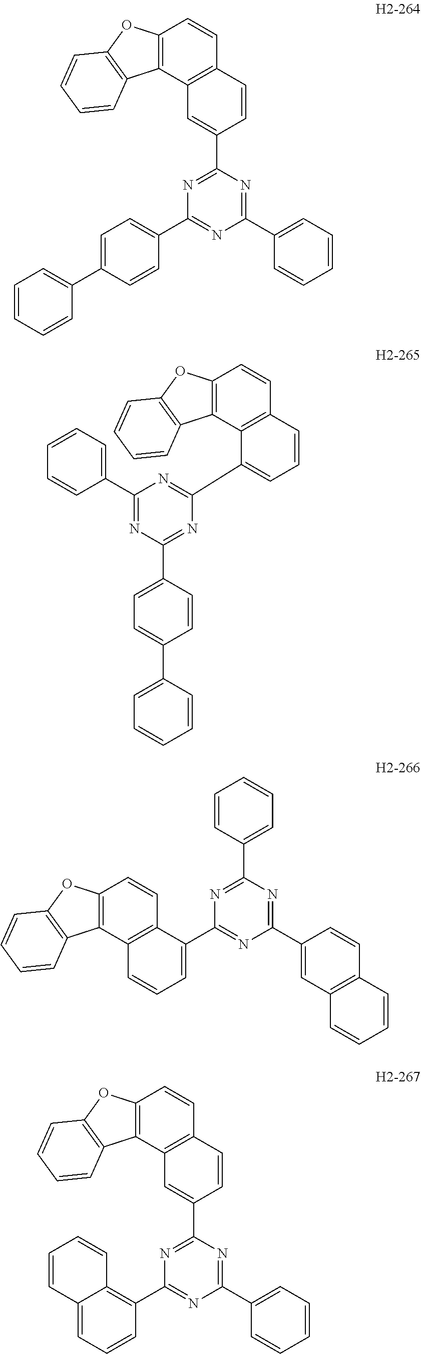 [SMILES: c1ccc(-c2ccc(-c3nc(-c4ccccc4)nc(-c4ccc5ccc6oc7ccccc7c6c5c4)n3)cc2)cc1 ; c1ccc(-c2ccc(-c3nc(-c4ccccc4)nc(-c4cccc5ccc6oc7ccccc7c6c45)n3)cc2)cc1 ; c1ccc(-c2nc(-c3ccc4ccccc4c3)nc(-c3cccc4c3ccc3oc5ccccc5c34)n2)cc1 ; c1ccc(-c2nc(-c3ccc4ccc5oc6ccccc6c5c4c3)nc(-c3cccc4ccccc34)n2)cc1]

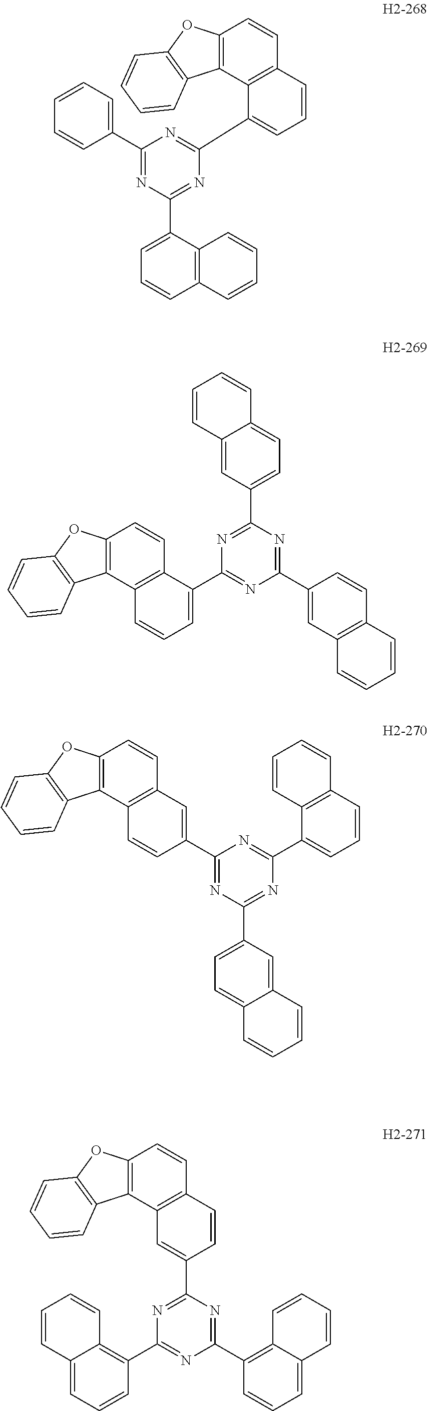 [SMILES: c1ccc(-c2nc(-c3cccc4ccccc34)nc(-c3cccc4ccc5oc6ccccc6c5c34)n2)cc1 ; c1ccc2cc(-c3nc(-c4ccc5ccccc5c4)nc(-c4cccc5c4ccc4oc6ccccc6c45)n3)ccc2c1 ; c1ccc2cc(-c3nc(-c4ccc5c(ccc6oc7ccccc7c65)c4)nc(-c4cccc5ccccc45)n3)ccc2c1 ; c1ccc2c(-c3nc(-c4ccc5ccc6oc7ccccc7c6c5c4)nc(-c4cccc5ccccc45)n3)cccc2c1]

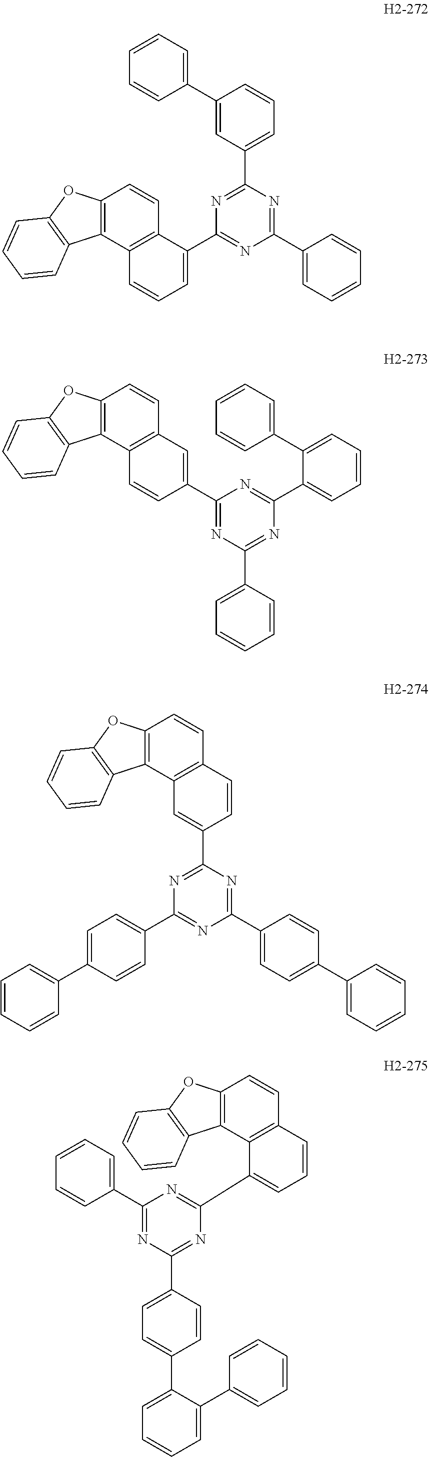 [SMILES: c1ccc(-c2cccc(-c3nc(-c4ccccc4)nc(-c4cccc5c4ccc4oc6ccccc6c45)n3)c2)cc1 ; c1ccc(-c2nc(-c3ccc4c(ccc5oc6ccccc6c54)c3)nc(-c3ccccc3-c3ccccc3)n2)cc1 ; c1ccc(-c2ccc(-c3nc(-c4ccc(-c5ccccc5)cc4)nc(-c4ccc5ccc6oc7ccccc7c6c5c4)n3)cc2)cc1 ; c1ccc(-c2nc(-c3ccc(-c4ccccc4-c4ccccc4)cc3)nc(-c3cccc4ccc5oc6ccccc6c5c34)n2)cc1]

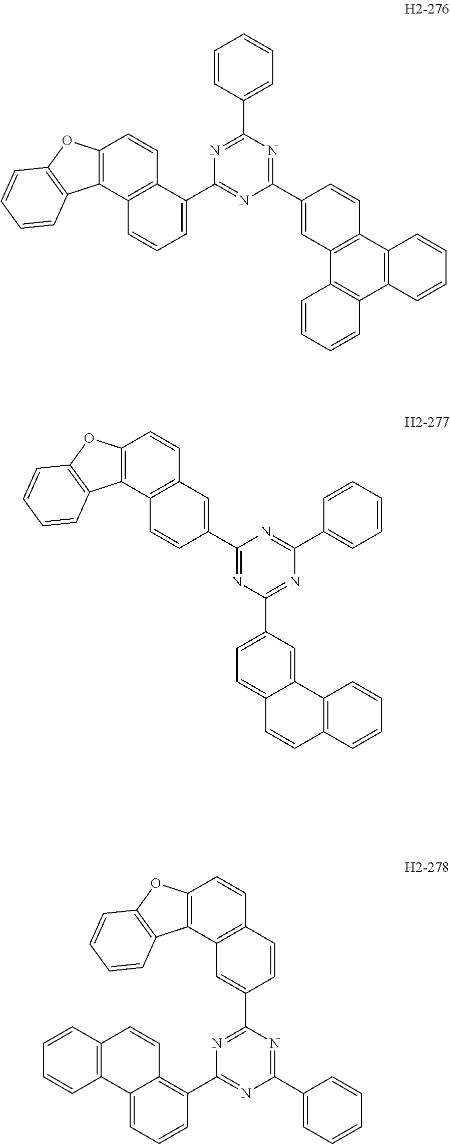 [SMILES: c1ccc(-c2nc(-c3ccc4c5ccccc5c5ccccc5c4c3)nc(-c3cccc4c3ccc3oc5ccccc5c34)n2)cc1 ; c1ccc(-c2nc(-c3ccc4c(ccc5oc6ccccc6c54)c3)nc(-c3ccc4ccc5ccccc5c4c3)n2)cc1 ; c1ccc(-c2nc(-c3ccc4ccc5oc6ccccc6c5c4c3)nc(-c3cccc4c3ccc3ccccc34)n2)cc1]

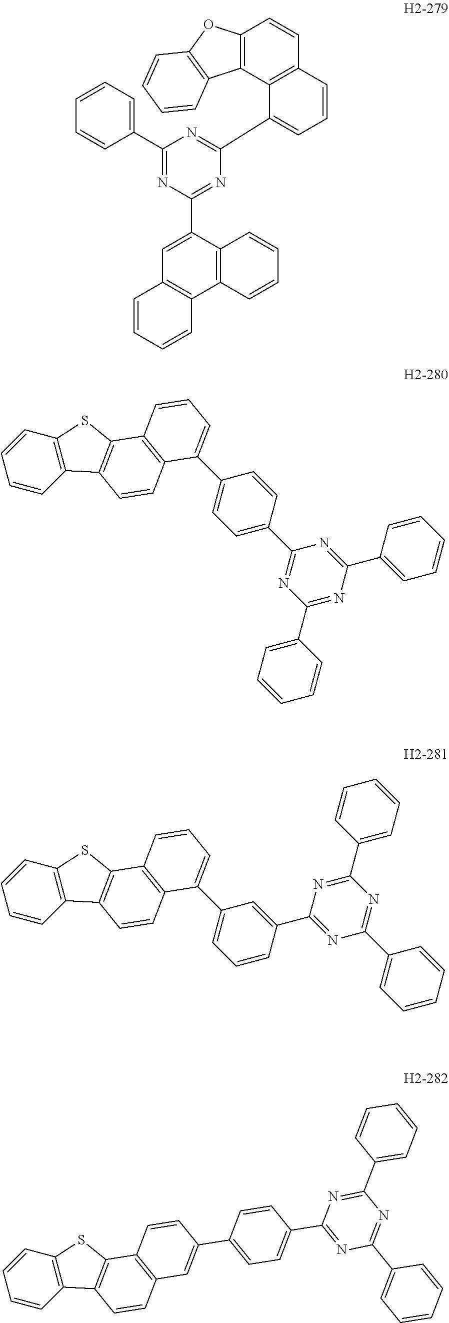 [SMILES: c1ccc(-c2nc(-c3cc4ccccc4c4ccccc34)nc(-c3cccc4ccc5oc6ccccc6c5c34)n2)cc1 ; c1ccc(-c2nc(-c3ccccc3)nc(-c3ccc(-c4cccc5c4ccc4c6ccccc6sc54)cc3)n2)cc1 ; c1ccc(-c2nc(-c3ccccc3)nc(-c3cccc(-c4cccc5c4ccc4c6ccccc6sc54)c3)n2)cc1 ; c1ccc(-c2nc(-c3ccccc3)nc(-c3ccc(-c4ccc5c(ccc6c7ccccc7sc56)c4)cc3)n2)cc1]

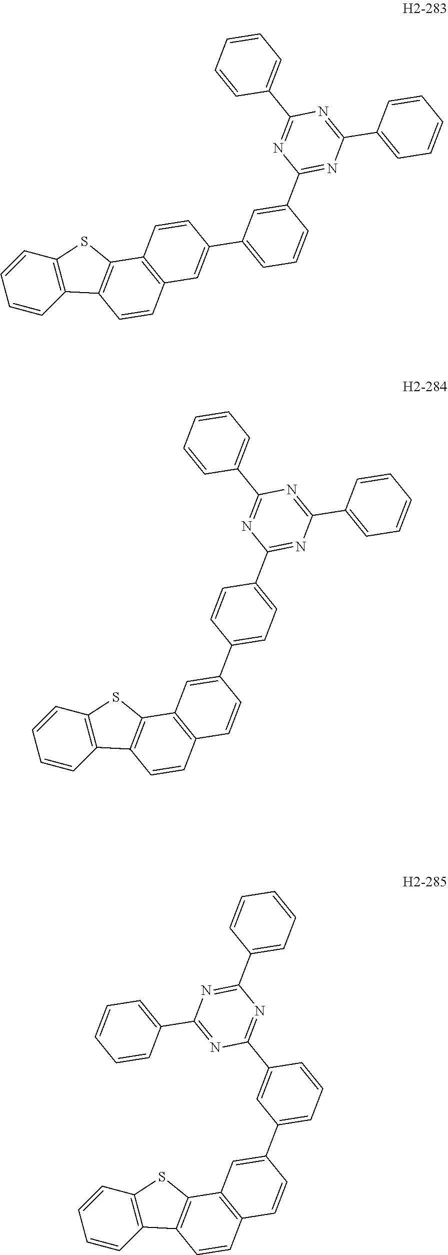 [SMILES: c1ccc(-c2nc(-c3ccccc3)nc(-c3cccc(-c4ccc5c(ccc6c7ccccc7sc56)c4)c3)n2)cc1 ; c1ccc(-c2nc(-c3ccccc3)nc(-c3ccc(-c4ccc5ccc6c7ccccc7sc6c5c4)cc3)n2)cc1 ; c1ccc(-c2nc(-c3ccccc3)nc(-c3cccc(-c4ccc5ccc6c7ccccc7sc6c5c4)c3)n2)cc1]

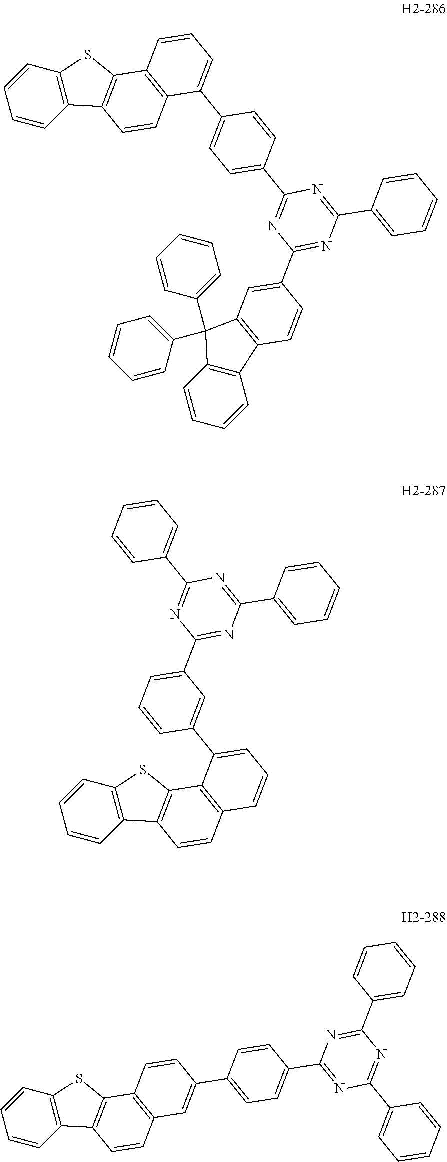 [SMILES: c1ccc(-c2nc(-c3ccc(-c4cccc5c4ccc4c6ccccc6sc54)cc3)nc(-c3ccc4c(c3)C(c3ccccc3)(c3ccccc3)c3ccccc3-4)n2)cc1 ; c1ccc(-c2nc(-c3ccccc3)nc(-c3cccc(-c4cccc5ccc6c7ccccc7sc6c45)c3)n2)cc1 ; c1ccc(-c2nc(-c3ccccc3)nc(-c3ccc(-c4ccc5c(ccc6c7ccccc7sc56)c4)cc3)n2)cc1]

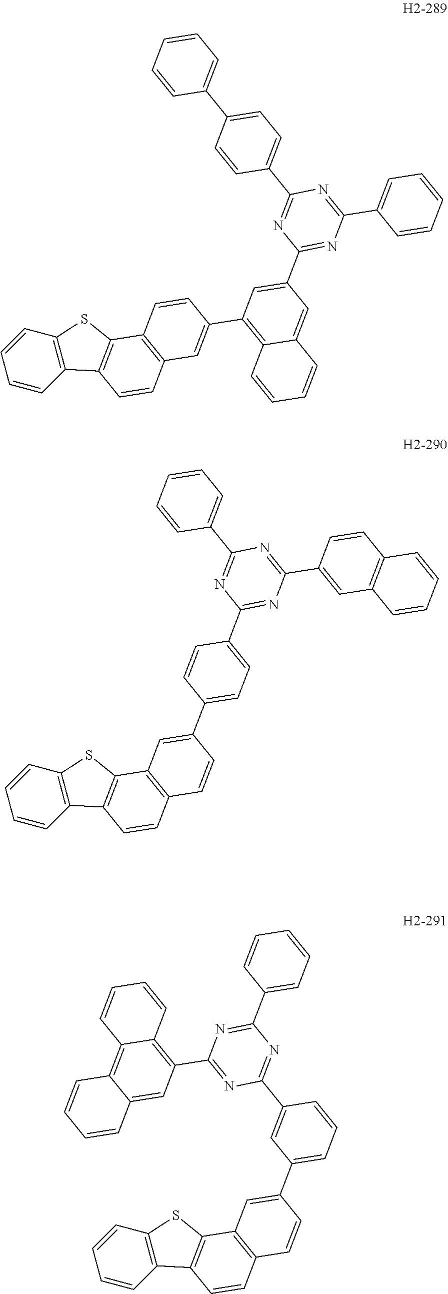 [SMILES: c1ccc(-c2ccc(-c3nc(-c4ccccc4)nc(-c4cc(-c5ccc6c(ccc7c8ccccc8sc67)c5)c5ccccc5c4)n3)cc2)cc1 ; c1ccc(-c2nc(-c3ccc(-c4ccc5ccc6c7ccccc7sc6c5c4)cc3)nc(-c3ccc4ccccc4c3)n2)cc1 ; c1ccc(-c2nc(-c3cccc(-c4ccc5ccc6c7ccccc7sc6c5c4)c3)nc(-c3cc4ccccc4c4ccccc34)n2)cc1]

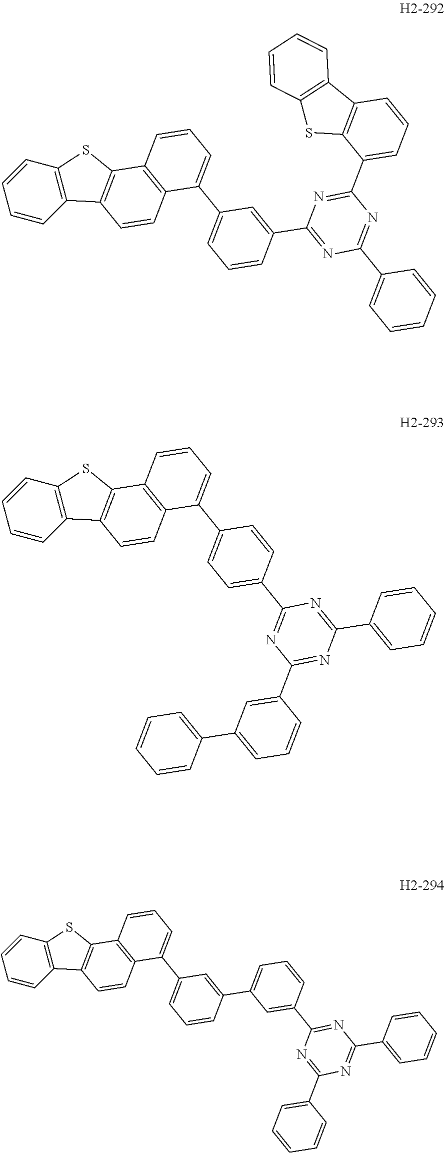 [SMILES: c1ccc(-c2nc(-c3cccc(-c4cccc5c4ccc4c6ccccc6sc54)c3)nc(-c3cccc4c3sc3ccccc34)n2)cc1 ; c1ccc(-c2cccc(-c3nc(-c4ccccc4)nc(-c4ccc(-c5cccc6c5ccc5c7ccccc7sc65)cc4)n3)c2)cc1 ; c1ccc(-c2nc(-c3ccccc3)nc(-c3cccc(-c4cccc(-c5cccc6c5ccc5c7ccccc7sc65)c4)c3)n2)cc1]

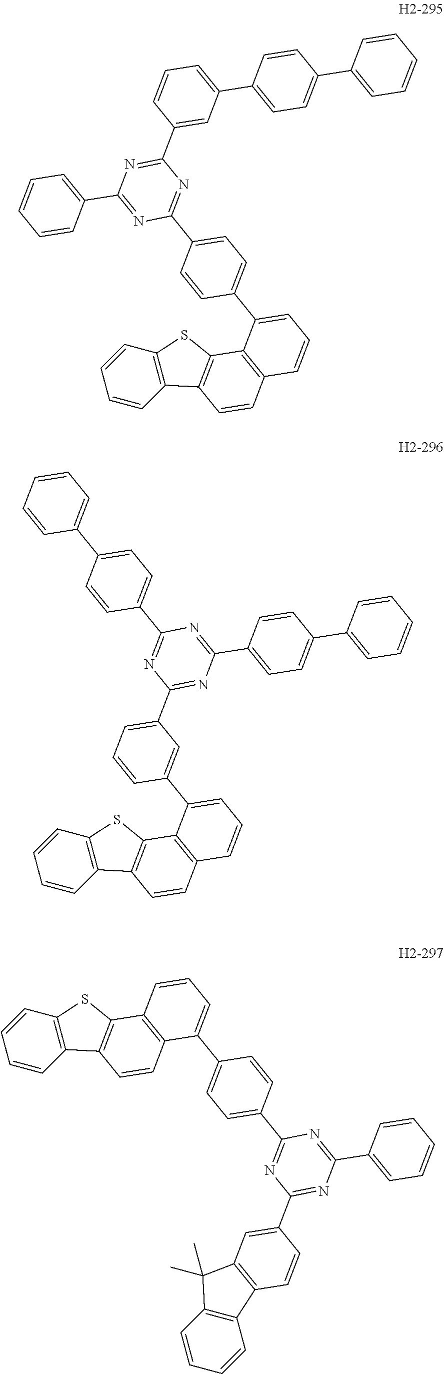 [SMILES: c1ccc(-c2ccc(-c3cccc(-c4nc(-c5ccccc5)nc(-c5ccc(-c6cccc7ccc8c9ccccc9sc8c67)cc5)n4)c3)cc2)cc1 ; c1ccc(-c2ccc(-c3nc(-c4ccc(-c5ccccc5)cc4)nc(-c4cccc(-c5cccc6ccc7c8ccccc8sc7c56)c4)n3)cc2)cc1 ; CC1(C)c2ccccc2-c2ccc(-c3nc(-c4ccccc4)nc(-c4ccc(-c5cccc6c5ccc5c7ccccc7sc65)cc4)n3)cc21]

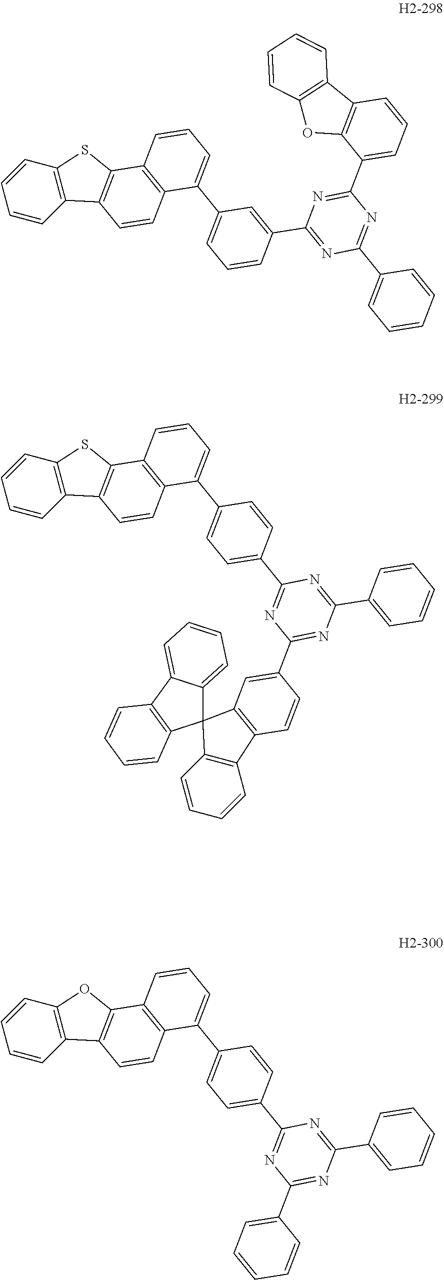 [SMILES: c1ccc(-c2nc(-c3cccc(-c4cccc5c4ccc4c6ccccc6sc54)c3)nc(-c3cccc4c3oc3ccccc34)n2)cc1 ; c1ccc(-c2nc(-c3ccc(-c4cccc5c4ccc4c6ccccc6sc54)cc3)nc(-c3ccc4c(c3)C3(c5ccccc5-c5ccccc53)c3ccccc3-4)n2)cc1 ; c1ccc(-c2nc(-c3ccccc3)nc(-c3ccc(-c4cccc5c4ccc4c6ccccc6oc54)cc3)n2)cc1]

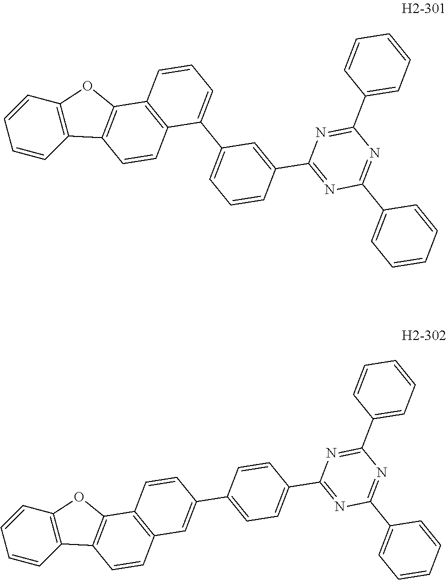 [SMILES: c1ccc(-c2nc(-c3ccccc3)nc(-c3cccc(-c4cccc5c4ccc4c6ccccc6oc54)c3)n2)cc1 ; c1ccc(-c2nc(-c3ccccc3)nc(-c3ccc(-c4ccc5c(ccc6c7ccccc7oc56)c4)cc3)n2)cc1]

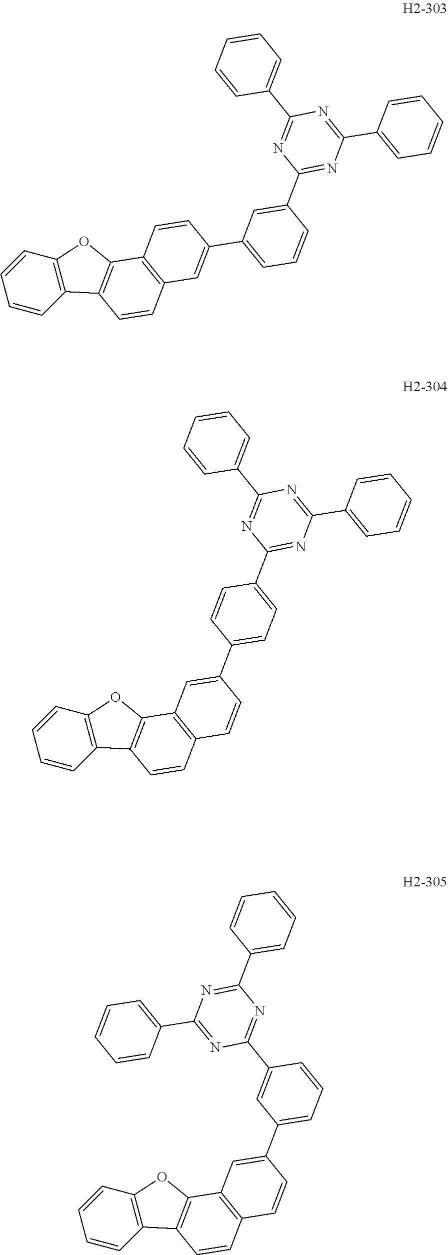 [SMILES: c1ccc(-c2nc(-c3ccccc3)nc(-c3cccc(-c4ccc5c(ccc6c7ccccc7oc56)c4)c3)n2)cc1 ; c1ccc(-c2nc(-c3ccccc3)nc(-c3ccc(-c4ccc5ccc6c7ccccc7oc6c5c4)cc3)n2)cc1 ; c1ccc(-c2nc(-c3ccccc3)nc(-c3cccc(-c4ccc5ccc6c7ccccc7oc6c5c4)c3)n2)cc1]

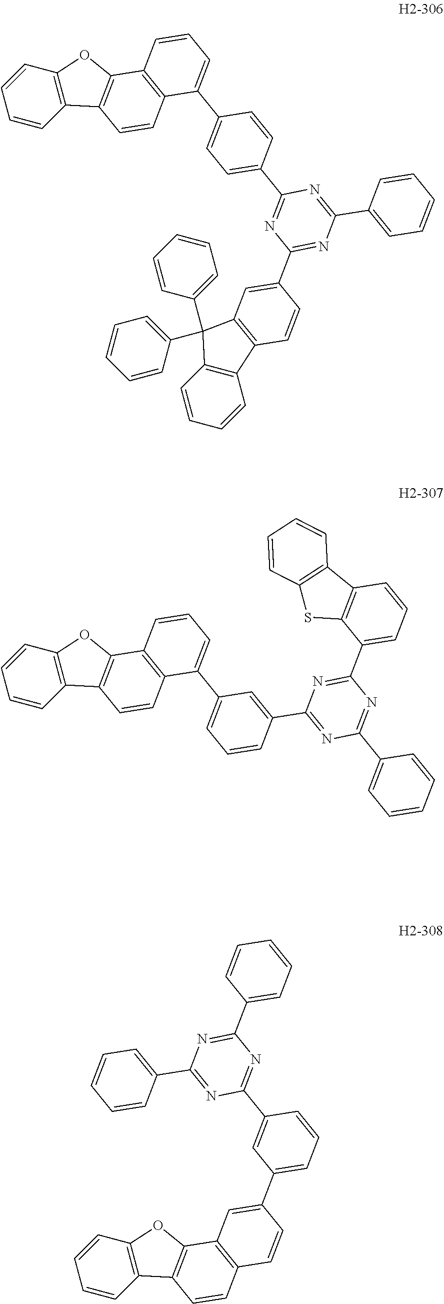 [SMILES: c1ccc(-c2nc(-c3ccc(-c4cccc5c4ccc4c6ccccc6oc54)cc3)nc(-c3ccc4c(c3)C(c3ccccc3)(c3ccccc3)c3ccccc3-4)n2)cc1 ; c1ccc(-c2nc(-c3cccc(-c4cccc5c4ccc4c6ccccc6oc54)c3)nc(-c3cccc4c3sc3ccccc34)n2)cc1 ; c1ccc(-c2nc(-c3ccccc3)nc(-c3cccc(-c4ccc5ccc6c7ccccc7oc6c5c4)c3)n2)cc1]

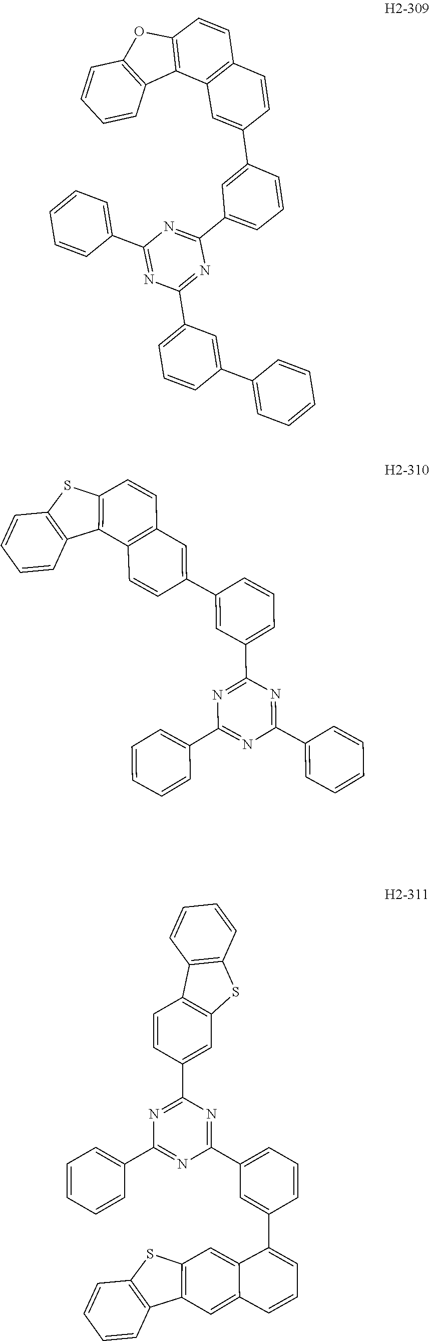

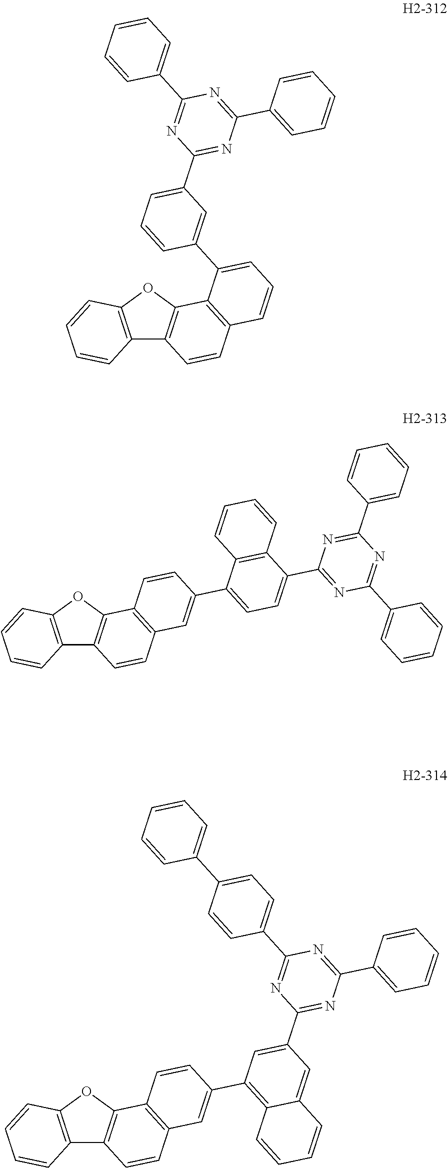 [SMILES: c1ccc(-c2nc(-c3ccccc3)nc(-c3cccc(-c4cccc5ccc6c7ccccc7oc6c45)c3)n2)cc1 ; c1ccc(-c2nc(-c3ccccc3)nc(-c3ccc(-c4ccc5c(ccc6c7ccccc7oc56)c4)c4ccccc34)n2)cc1 ; c1ccc(-c2ccc(-c3nc(-c4ccccc4)nc(-c4cc(-c5ccc6c(ccc7c8ccccc8oc67)c5)c5ccccc5c4)n3)cc2)cc1]

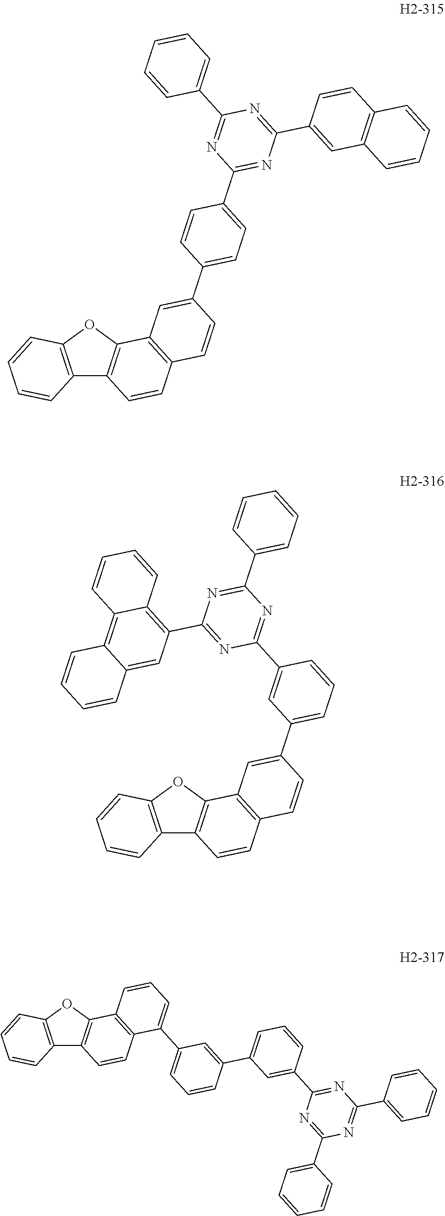 [SMILES: c1ccc(-c2nc(-c3ccc(-c4ccc5ccc6c7ccccc7oc6c5c4)cc3)nc(-c3ccc4ccccc4c3)n2)cc1 ; c1ccc(-c2nc(-c3cccc(-c4ccc5ccc6c7ccccc7oc6c5c4)c3)nc(-c3cc4ccccc4c4ccccc34)n2)cc1 ; c1ccc(-c2nc(-c3ccccc3)nc(-c3cccc(-c4cccc(-c5cccc6c5ccc5c7ccccc7oc65)c4)c3)n2)cc1]

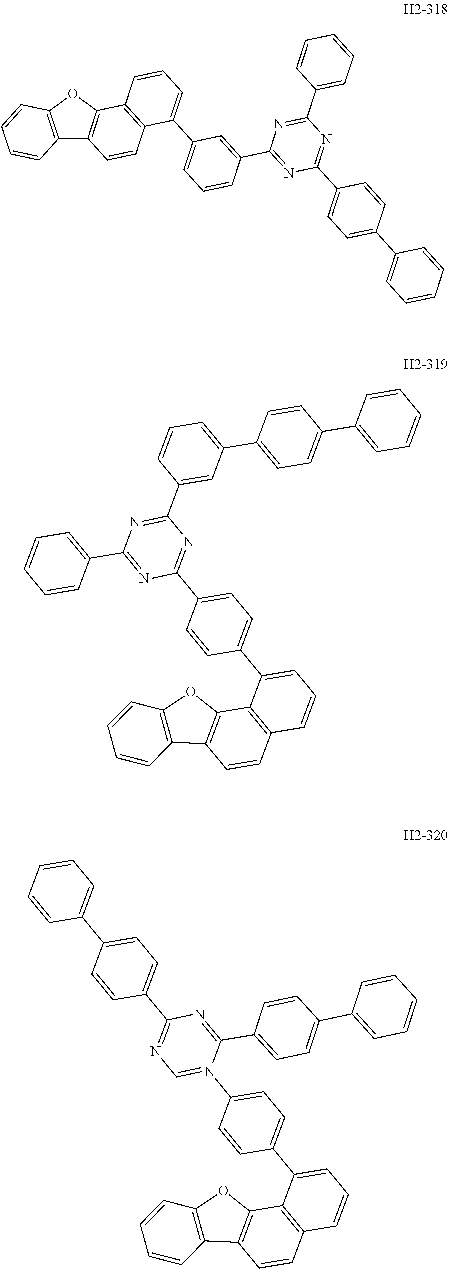

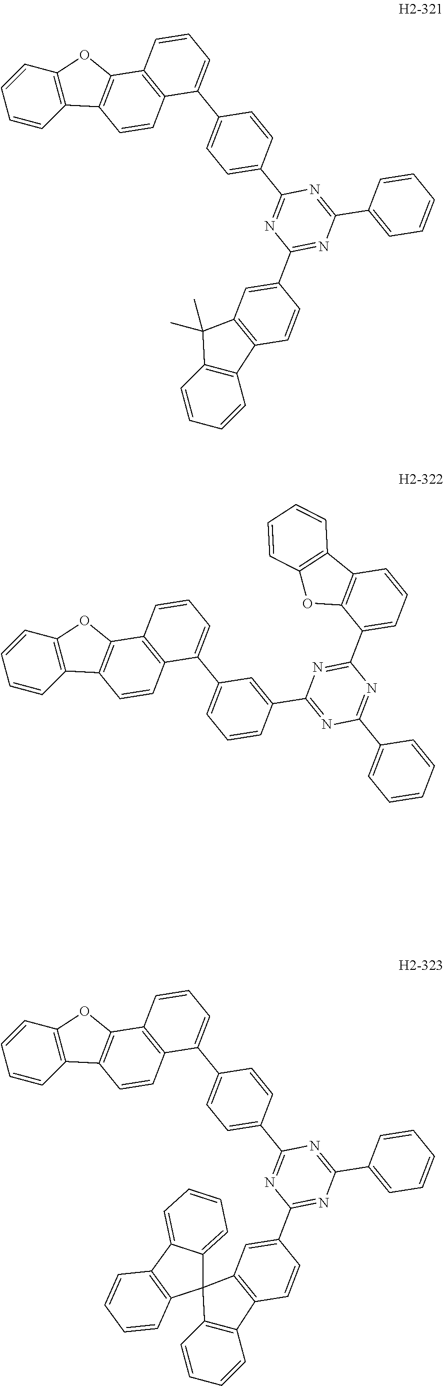 [SMILES: CC1(C)c2ccccc2-c2ccc(-c3nc(-c4ccccc4)nc(-c4ccc(-c5cccc6c5ccc5c7ccccc7oc65)cc4)n3)cc21 ; c1ccc(-c2nc(-c3cccc(-c4cccc5c4ccc4c6ccccc6oc54)c3)nc(-c3cccc4c3oc3ccccc34)n2)cc1 ; c1ccc(-c2nc(-c3ccc(-c4cccc5c4ccc4c6ccccc6oc54)cc3)nc(-c3ccc4c(c3)C3(c5ccccc5-c5ccccc53)c3ccccc3-4)n2)cc1]

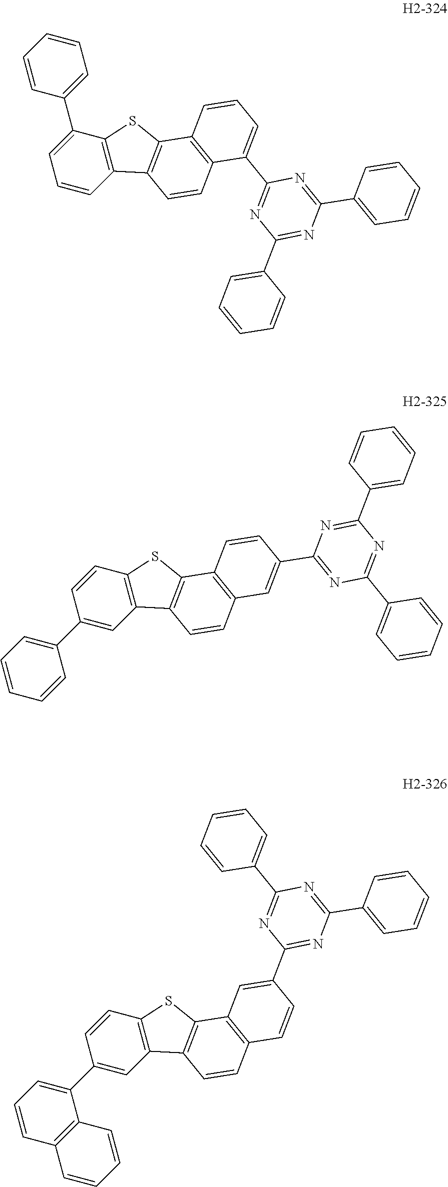 [SMILES: c1ccc(-c2nc(-c3ccccc3)nc(-c3cccc4c3ccc3c5cccc(-c6ccccc6)c5sc43)n2)cc1 ; c1ccc(-c2ccc3sc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6sc5c4c3)n2)cc1]

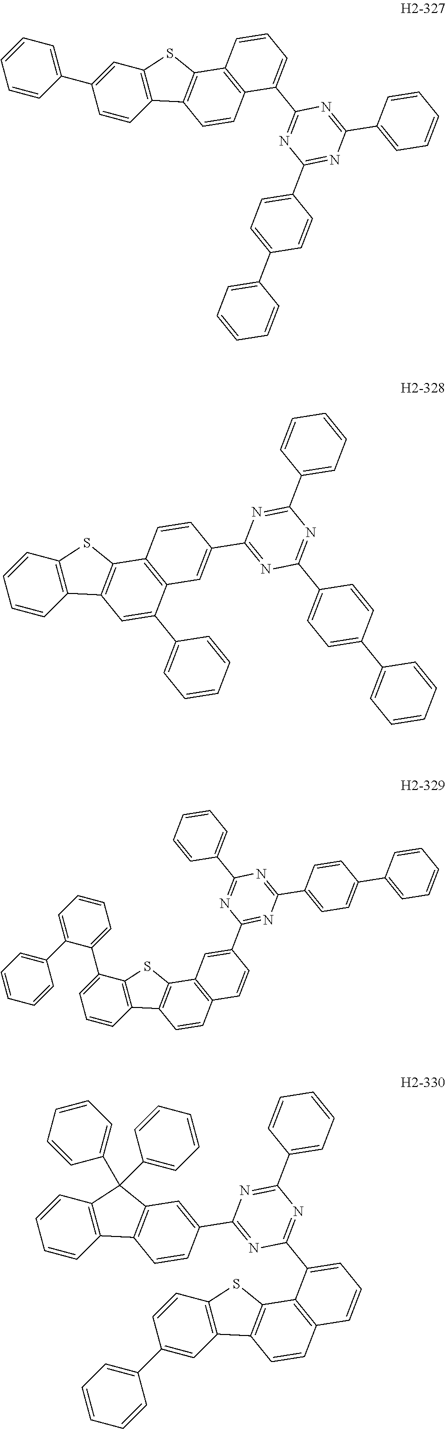 [SMILES: c1ccc(-c2ccc(-c3nc(-c4ccccc4)nc(-c4cccc5c4ccc4c6ccc(-c7ccccc7)cc6sc54)n3)cc2)cc1 ; c1ccc(-c2ccc(-c3nc(-c4ccccc4)nc(-c4ccc5c(c4)c(-c4ccccc4)cc4c6ccccc6sc54)n3)cc2)cc1 ; c1ccc(-c2ccc(-c3nc(-c4ccccc4)nc(-c4ccc5ccc6c7cccc(-c8ccccc8-c8ccccc8)c7sc6c5c4)n3)cc2)cc1 ; c1ccc(-c2ccc3sc4c(ccc5cccc(-c6nc(-c7ccccc7)nc(-c7ccc8c(c7)C(c7ccccc7)(c7ccccc7)c7ccccc7-8)n6)c54)c3c2)cc1]

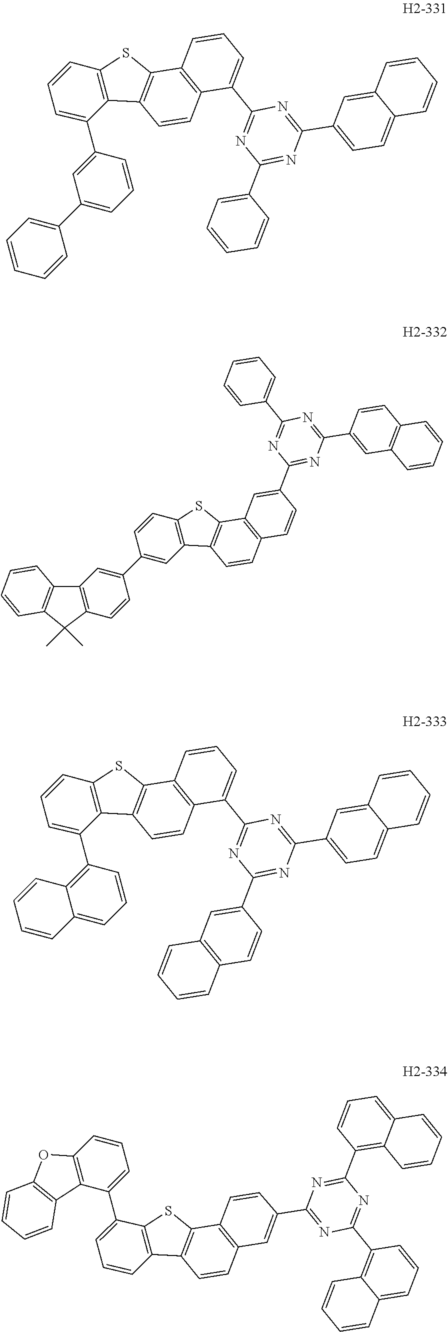 [SMILES: c1ccc(-c2cccc(-c3cccc4sc5c6cccc(-c7nc(-c8ccccc8)nc(-c8ccc9ccccc9c8)n7)c6ccc5c34)c2)cc1 ; CC1(C)c2ccccc2-c2cc(-c3ccc4sc5c6cc(-c7nc(-c8ccccc8)nc(-c8ccc9ccccc9c8)n7)ccc6ccc5c4c3)ccc21 ; c1ccc2cc(-c3nc(-c4ccc5ccccc5c4)nc(-c4cccc5c4ccc4c5sc5cccc(-c6cccc7ccccc67)c54)n3)ccc2c1 ; c1ccc2c(-c3nc(-c4ccc5c(ccc6c7cccc(-c8cccc9oc%10ccccc%10c89)c7sc56)c4)nc(-c4cccc5ccccc45)n3)cccc2c1]

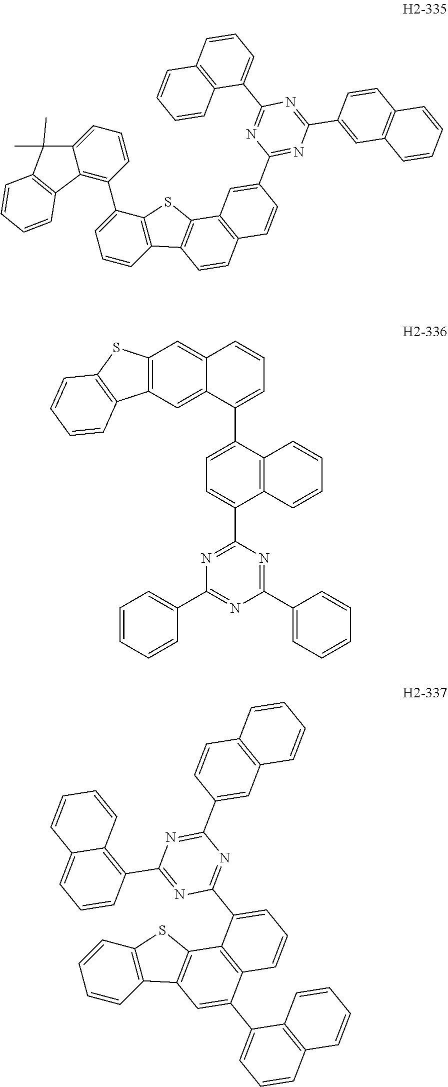 [SMILES: CC1(C)c2ccccc2-c2c(-c3cccc4c3sc3c5cc(-c6nc(-c7ccc8ccccc8c7)nc(-c7cccc8ccccc78)n6)ccc5ccc43)cccc21 ; c1ccc(-c2nc(-c3ccccc3)nc(-c3ccc(-c4cccc5cc6sc7ccccc7c6cc45)c4ccccc34)n2)cc1 ; c1ccc2cc(-c3nc(-c4cccc5ccccc45)nc(-c4cccc5c(-c6cccc7ccccc67)cc6c7ccccc7sc6c45)n3)ccc2c1]

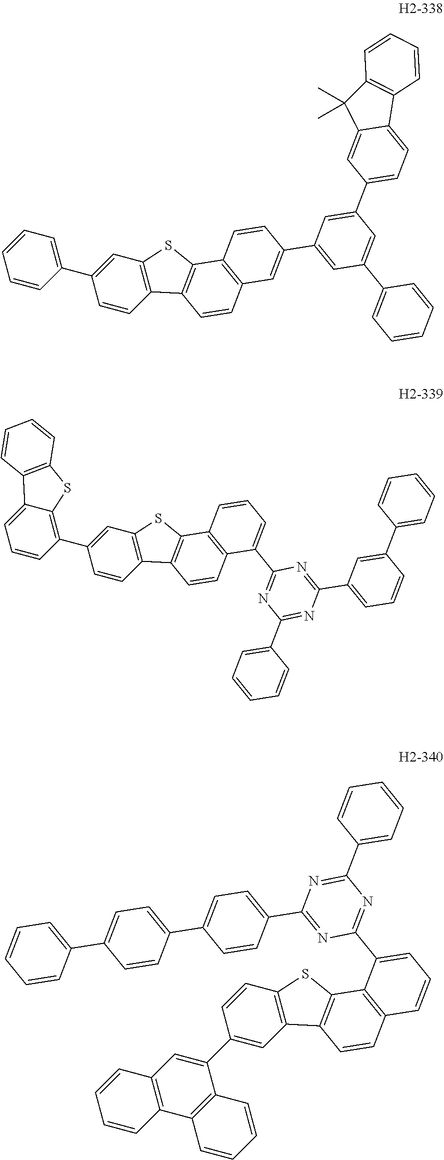 [SMILES: CC1(C)c2ccccc2-c2ccc(-c3cc(-c4ccccc4)cc(-c4ccc5c(ccc6c7ccc(-c8ccccc8)cc7sc56)c4)c3)cc21 ; c1ccc(-c2cccc(-c3nc(-c4ccccc4)nc(-c4cccc5c4ccc4c6ccc(-c7cccc8c7sc7ccccc78)cc6sc54)n3)c2)cc1 ; c1ccc(-c2ccc(-c3ccc(-c4nc(-c5ccccc5)nc(-c5cccc6ccc7c8cc(-c9cc%10ccccc%10c%10ccccc9%10)ccc8sc7c56)n4)cc3)cc2)cc1]

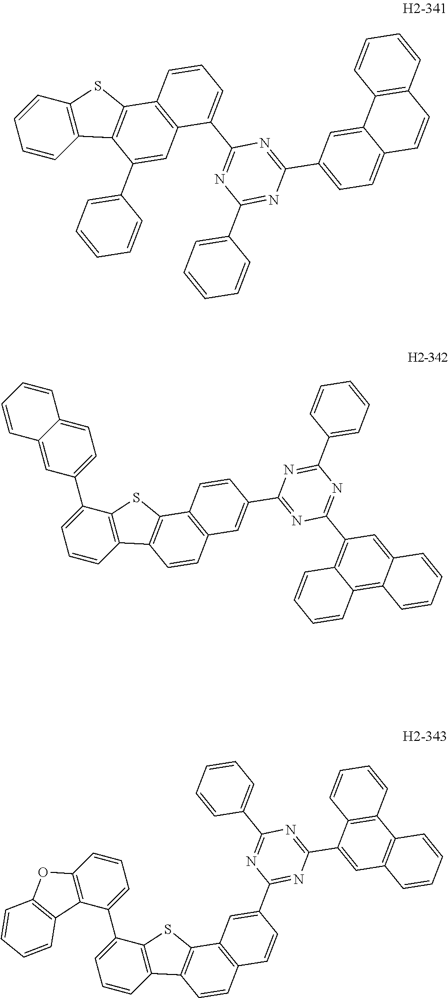 [SMILES: c1ccc(-c2nc(-c3ccc4ccc5ccccc5c4c3)nc(-c3cccc4c3cc(-c3ccccc3)c3c5ccccc5sc43)n2)cc1 ; c1ccc(-c2nc(-c3ccc4c(ccc5c6cccc(-c7ccc8ccccc8c7)c6sc45)c3)nc(-c3cc4ccccc4c4ccccc34)n2)cc1 ; c1ccc(-c2nc(-c3ccc4ccc5c6cccc(-c7cccc8oc9ccccc9c78)c6sc5c4c3)nc(-c3cc4ccccc4c4ccccc34)n2)cc1]

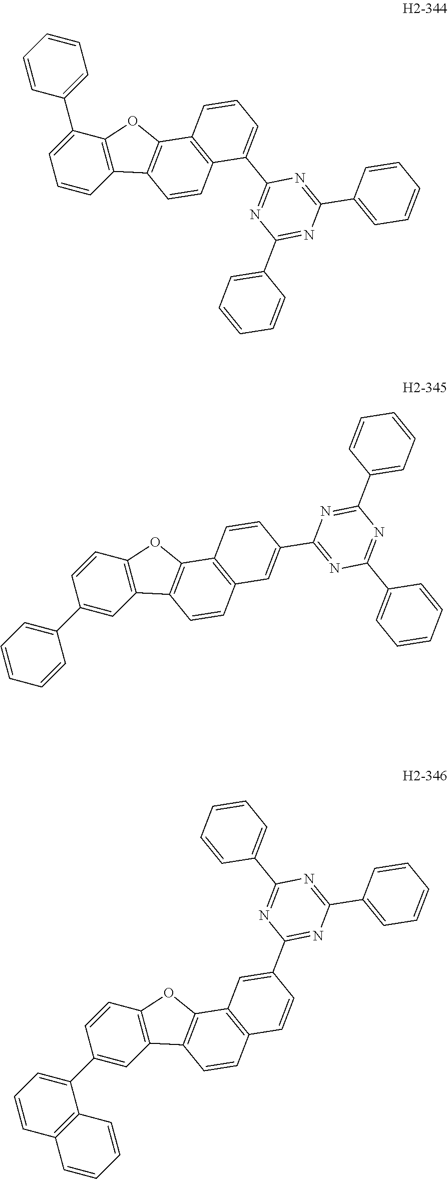 [SMILES: c1ccc(-c2nc(-c3ccccc3)nc(-c3cccc4c3ccc3c5cccc(-c6ccccc6)c5oc43)n2)cc1 ; c1ccc(-c2ccc3oc4c5ccc(-c6nc(-c7ccccc7)nc(-c7ccccc7)n6)cc5ccc4c3c2)cc1 ; c1ccc(-c2nc(-c3ccccc3)nc(-c3ccc4ccc5c6cc(-c7cccc8ccccc78)ccc6oc5c4c3)n2)cc1]

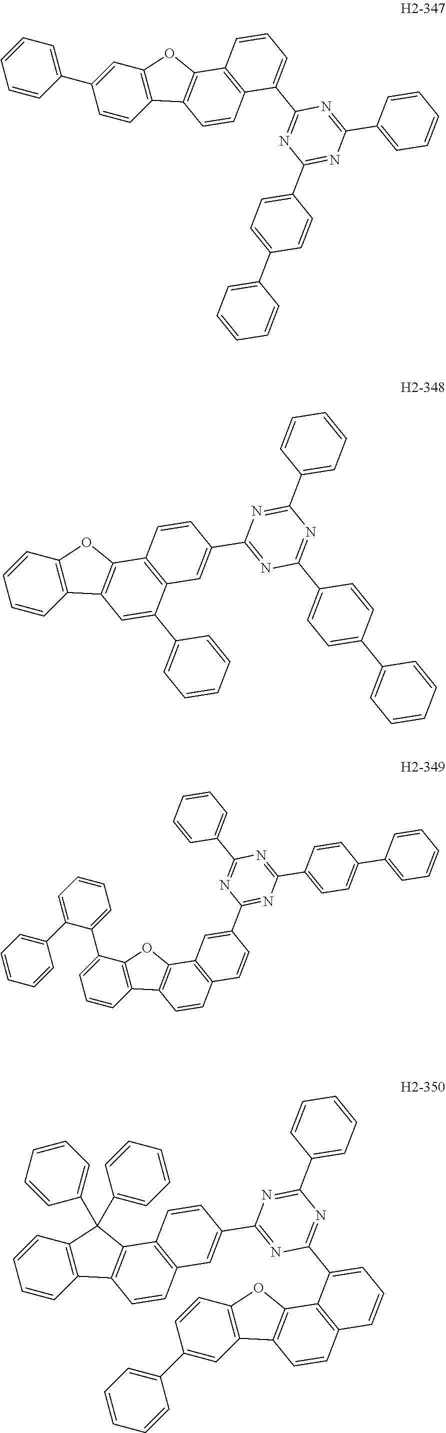 [SMILES: c1ccc(-c2ccc(-c3nc(-c4ccccc4)nc(-c4cccc5c4ccc4c6ccc(-c7ccccc7)cc6oc54)n3)cc2)cc1 ; c1ccc(-c2ccc(-c3nc(-c4ccccc4)nc(-c4ccc5c(c4)c(-c4ccccc4)cc4c6ccccc6oc54)n3)cc2)cc1 ; c1ccc(-c2ccc(-c3nc(-c4ccccc4)nc(-c4ccc5ccc6c7cccc(-c8ccccc8-c8ccccc8)c7oc6c5c4)n3)cc2)cc1 ; c1ccc(-c2ccc3oc4c(ccc5cccc(-c6nc(-c7ccccc7)nc(-c7ccc8c9c(ccc8c7)-c7ccccc7C9(c7ccccc7)c7ccccc7)n6)c54)c3c2)cc1]

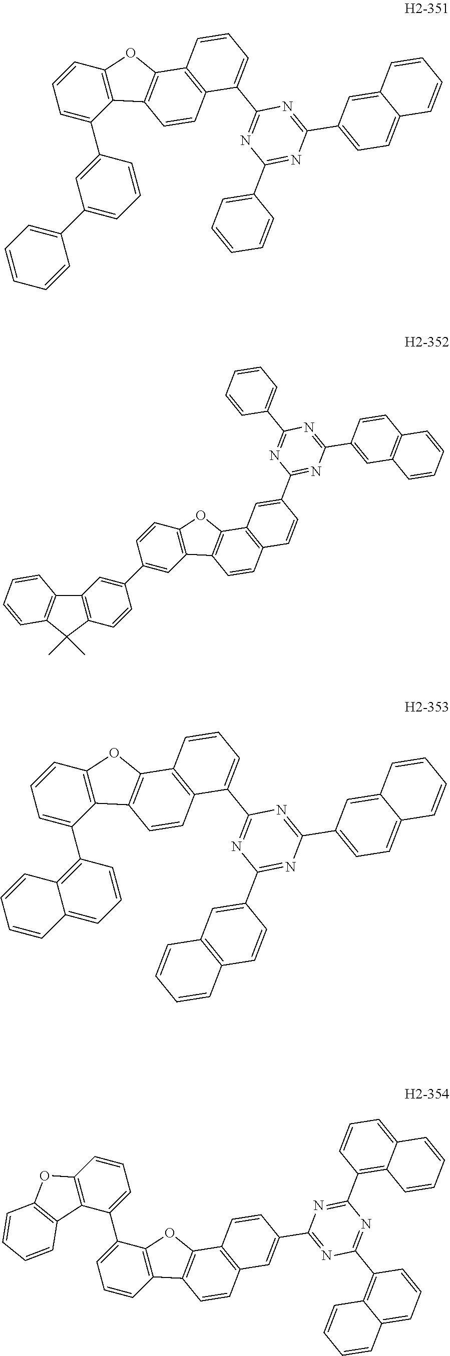 [SMILES: c1ccc(-c2cccc(-c3cccc4oc5c6cccc(-c7nc(-c8ccccc8)nc(-c8ccc9ccccc9c8)n7)c6ccc5c34)c2)cc1 ; CC1(C)c2ccccc2-c2cc(-c3ccc4oc5c6cc(-c7nc(-c8ccccc8)nc(-c8ccc9ccccc9c8)n7)ccc6ccc5c4c3)ccc21 ; c1ccc2cc(-c3nc(-c4ccc5ccccc5c4)nc(-c4cccc5c4ccc4c5oc5cccc(-c6cccc7ccccc67)c54)n3)ccc2c1 ; c1ccc2c(-c3nc(-c4ccc5c(ccc6c7cccc(-c8cccc9oc%10ccccc%10c89)c7oc56)c4)nc(-c4cccc5ccccc45)n3)cccc2c1]

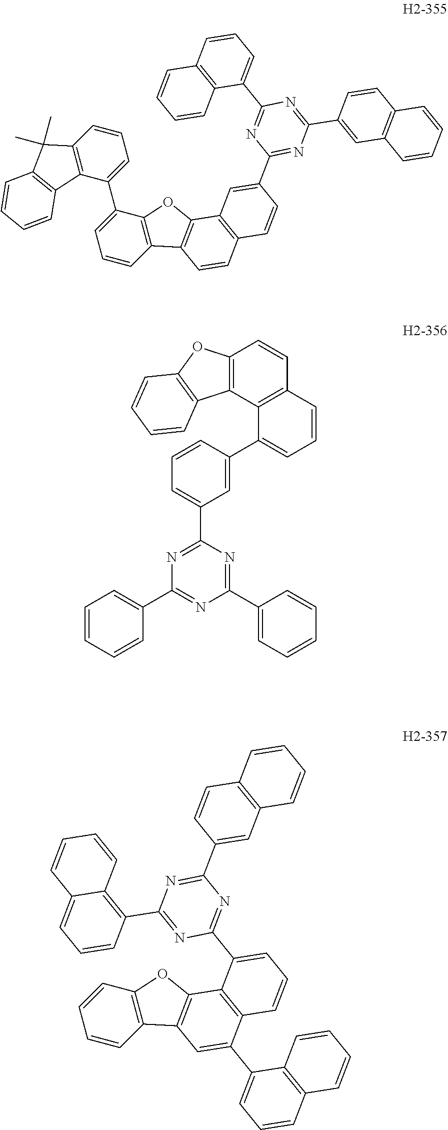 [SMILES: CC1(C)c2ccccc2-c2c(-c3cccc4c3oc3c5cc(-c6nc(-c7ccc8ccccc8c7)nc(-c7cccc8ccccc78)n6)ccc5ccc43)cccc21 ; c1ccc(-c2nc(-c3ccccc3)nc(-c3cccc(-c4cccc5ccc6oc7ccccc7c6c45)c3)n2)cc1 ; c1ccc2cc(-c3nc(-c4cccc5ccccc45)nc(-c4cccc5c(-c6cccc7ccccc67)cc6c7ccccc7oc6c45)n3)ccc2c1]

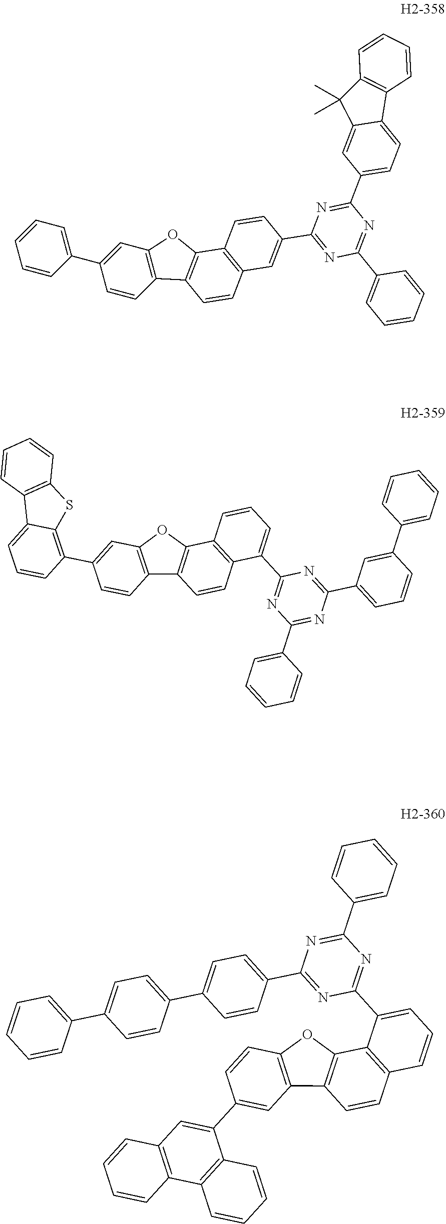 [SMILES: CC1(C)c2ccccc2-c2ccc(-c3nc(-c4ccccc4)nc(-c4ccc5c(ccc6c7ccc(-c8ccccc8)cc7oc56)c4)n3)cc21 ; c1ccc(-c2cccc(-c3nc(-c4ccccc4)nc(-c4cccc5c4ccc4c6ccc(-c7cccc8c7sc7ccccc78)cc6oc54)n3)c2)cc1 ; c1ccc(-c2ccc(-c3ccc(-c4nc(-c5ccccc5)nc(-c5cccc6ccc7c8cc(-c9cc%10ccccc%10c%10ccccc9%10)ccc8oc7c56)n4)cc3)cc2)cc1]

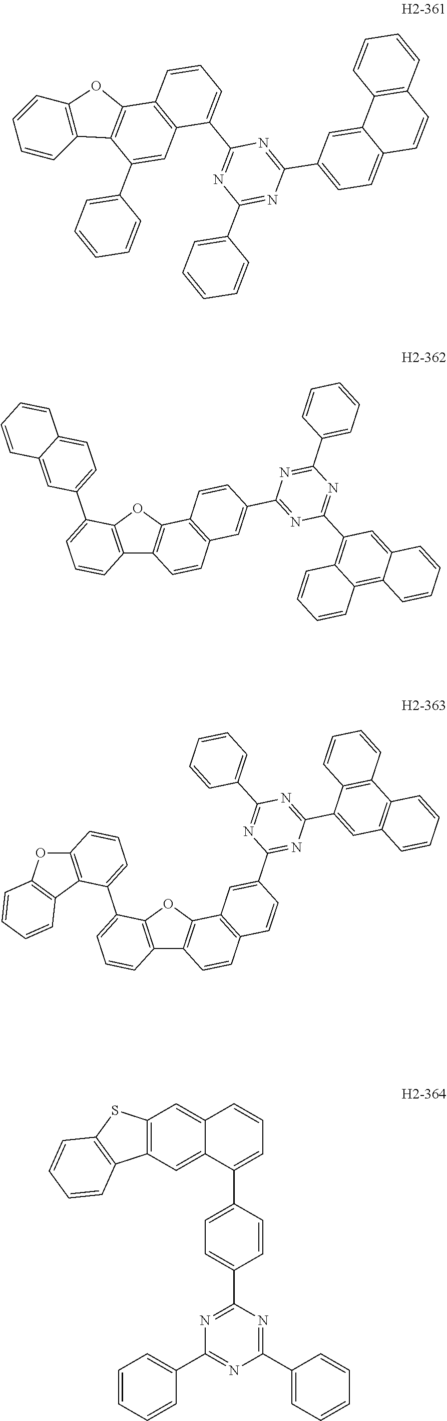 [SMILES: c1ccc(-c2nc(-c3ccc4ccc5ccccc5c4c3)nc(-c3cccc4c3cc(-c3ccccc3)c3c5ccccc5oc43)n2)cc1 ; c1ccc(-c2nc(-c3ccc4c(ccc5c6cccc(-c7ccc8ccccc8c7)c6oc45)c3)nc(-c3cc4ccccc4c4ccccc34)n2)cc1 ; c1ccc(-c2nc(-c3ccc4ccc5c6cccc(-c7cccc8oc9ccccc9c78)c6oc5c4c3)nc(-c3cc4ccccc4c4ccccc34)n2)cc1 ; c1ccc(-c2nc(-c3ccccc3)nc(-c3ccc(-c4cccc5cc6sc7ccccc7c6cc45)cc3)n2)cc1]

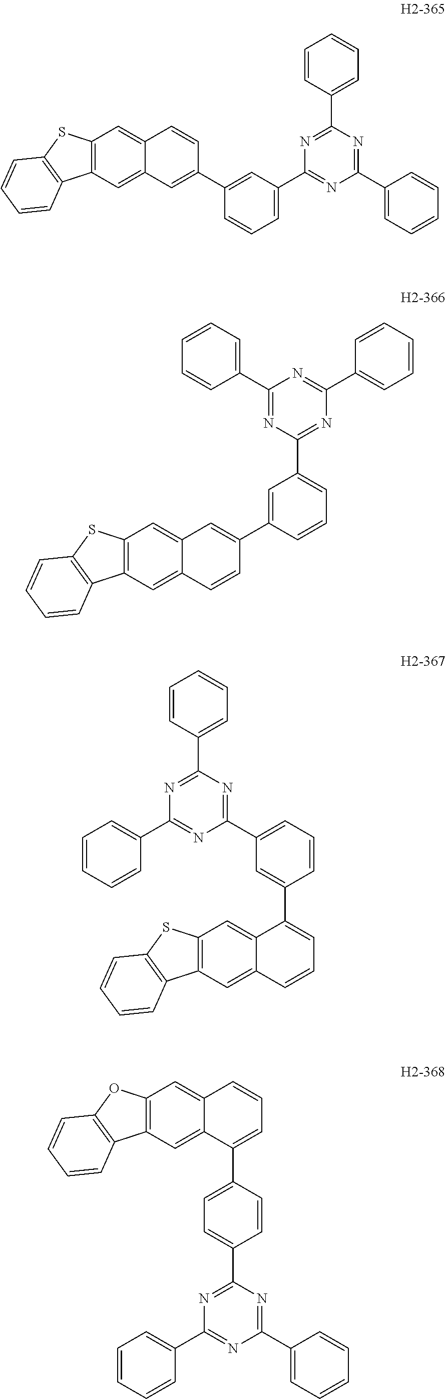 [SMILES: c1ccc(-c2nc(-c3ccccc3)nc(-c3cccc(-c4ccc5cc6sc7ccccc7c6cc5c4)c3)n2)cc1 ; c1ccc(-c2nc(-c3ccccc3)nc(-c3cccc(-c4ccc5cc6c(cc5c4)sc4ccccc46)c3)n2)cc1 ; c1ccc(-c2nc(-c3ccccc3)nc(-c3cccc(-c4cccc5cc6c(cc45)sc4ccccc46)c3)n2)cc1 ; c1ccc(-c2nc(-c3ccccc3)nc(-c3ccc(-c4cccc5cc6oc7ccccc7c6cc45)cc3)n2)cc1]

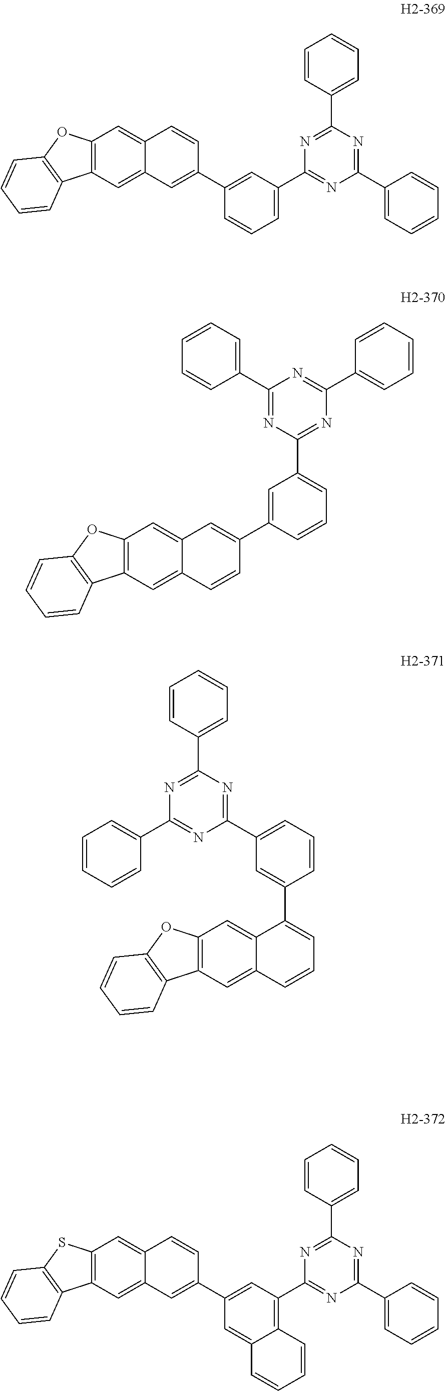 [SMILES: c1ccc(-c2nc(-c3ccccc3)nc(-c3cccc(-c4ccc5cc6oc7ccccc7c6cc5c4)c3)n2)cc1 ; c1ccc(-c2nc(-c3ccccc3)nc(-c3cccc(-c4ccc5cc6c(cc5c4)oc4ccccc46)c3)n2)cc1 ; c1ccc(-c2nc(-c3ccccc3)nc(-c3cccc(-c4cccc5cc6c(cc45)oc4ccccc46)c3)n2)cc1 ; c1ccc(-c2nc(-c3ccccc3)nc(-c3cc(-c4ccc5cc6sc7ccccc7c6cc5c4)cc4ccccc34)n2)cc1]

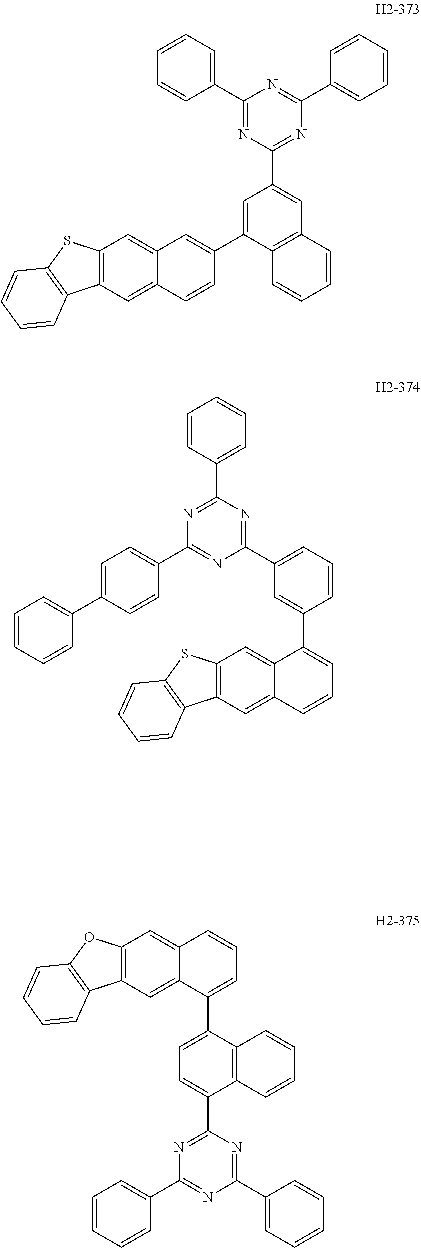 [SMILES: c1ccc(-c2nc(-c3ccccc3)nc(-c3cc(-c4ccc5cc6c(cc5c4)sc4ccccc46)c4ccccc4c3)n2)cc1 ; c1ccc(-c2ccc(-c3nc(-c4ccccc4)nc(-c4cccc(-c5cccc6cc7c(cc56)sc5ccccc57)c4)n3)cc2)cc1 ; c1ccc(-c2nc(-c3ccccc3)nc(-c3ccc(-c4cccc5cc6oc7ccccc7c6cc45)c4ccccc34)n2)cc1]

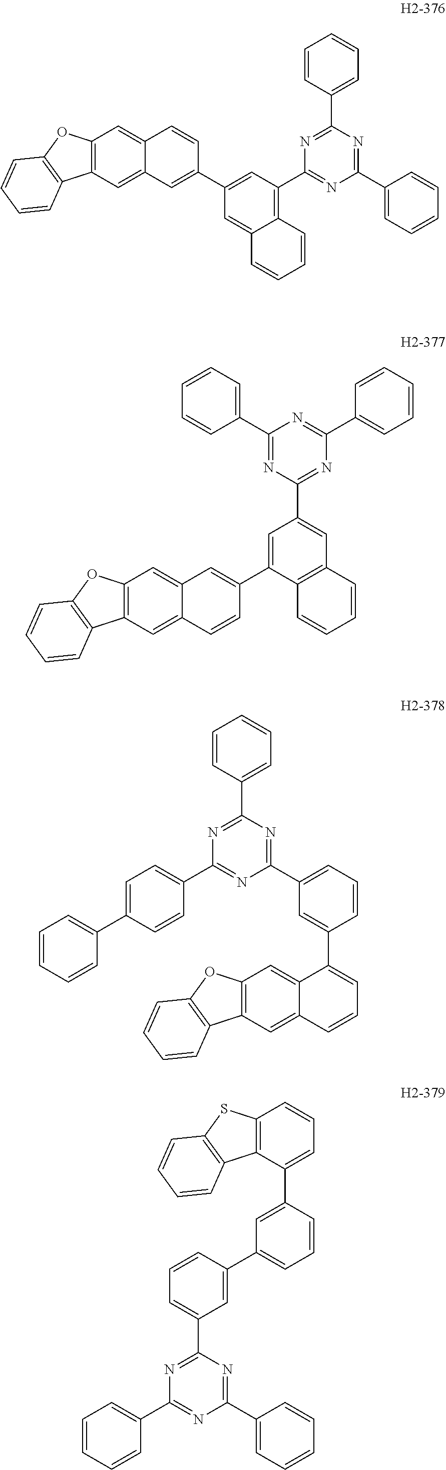 [SMILES: c1ccc(-c2nc(-c3ccccc3)nc(-c3cc(-c4ccc5cc6oc7ccccc7c6cc5c4)cc4ccccc34)n2)cc1 ; c1ccc(-c2nc(-c3ccccc3)nc(-c3cc(-c4ccc5cc6c(cc5c4)oc4ccccc46)c4ccccc4c3)n2)cc1 ; c1ccc(-c2ccc(-c3nc(-c4ccccc4)nc(-c4cccc(-c5cccc6cc7c(cc56)oc5ccccc57)c4)n3)cc2)cc1 ; c1ccc(-c2nc(-c3ccccc3)nc(-c3cccc(-c4cccc(-c5cccc6sc7ccccc7c56)c4)c3)n2)cc1]

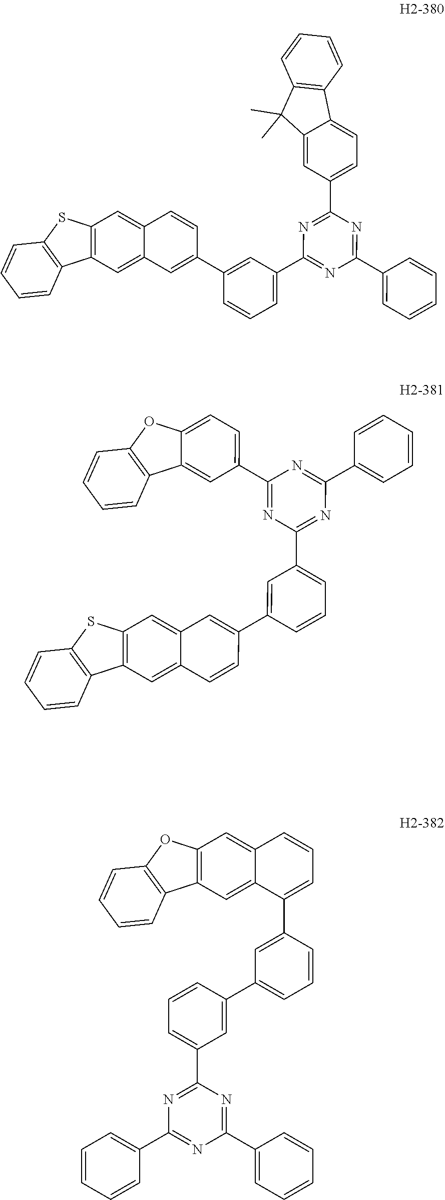 [SMILES: CC1(C)c2ccccc2-c2ccc(-c3nc(-c4ccccc4)nc(-c4cccc(-c5ccc6cc7sc8ccccc8c7cc6c5)c4)n3)cc21 ; c1ccc(-c2nc(-c3cccc(-c4ccc5cc6c(cc5c4)sc4ccccc46)c3)nc(-c3ccc4oc5ccccc5c4c3)n2)cc1 ; c1ccc(-c2nc(-c3ccccc3)nc(-c3cccc(-c4cccc(-c5cccc6cc7oc8ccccc8c7cc56)c4)c3)n2)cc1]

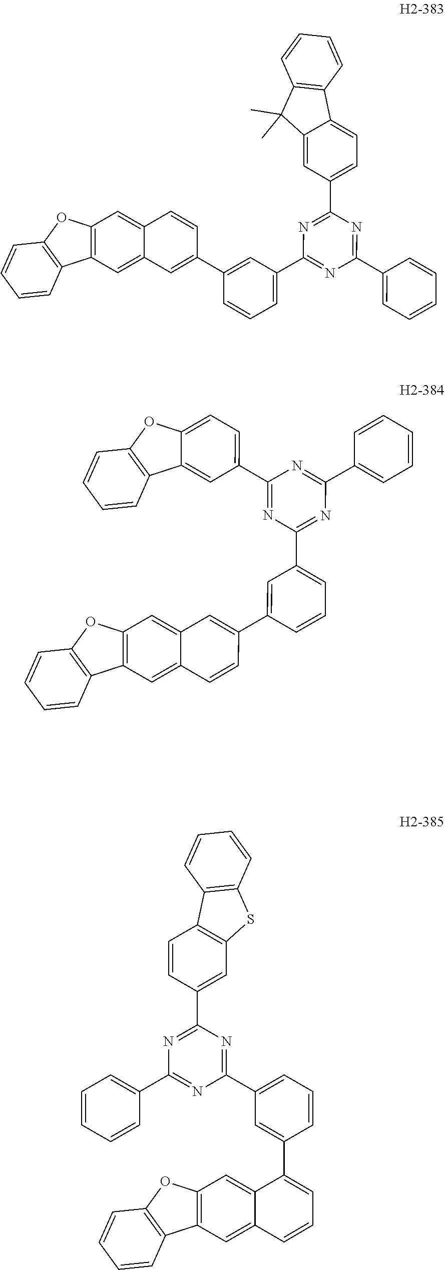 [SMILES: CC1(C)c2ccccc2-c2ccc(-c3nc(-c4ccccc4)nc(-c4cccc(-c5ccc6cc7oc8ccccc8c7cc6c5)c4)n3)cc21 ; c1ccc(-c2nc(-c3cccc(-c4ccc5cc6c(cc5c4)oc4ccccc46)c3)nc(-c3ccc4oc5ccccc5c4c3)n2)cc1 ; c1ccc(-c2nc(-c3cccc(-c4cccc5cc6c(cc45)oc4ccccc46)c3)nc(-c3ccc4c(c3)sc3ccccc34)n2)cc1]

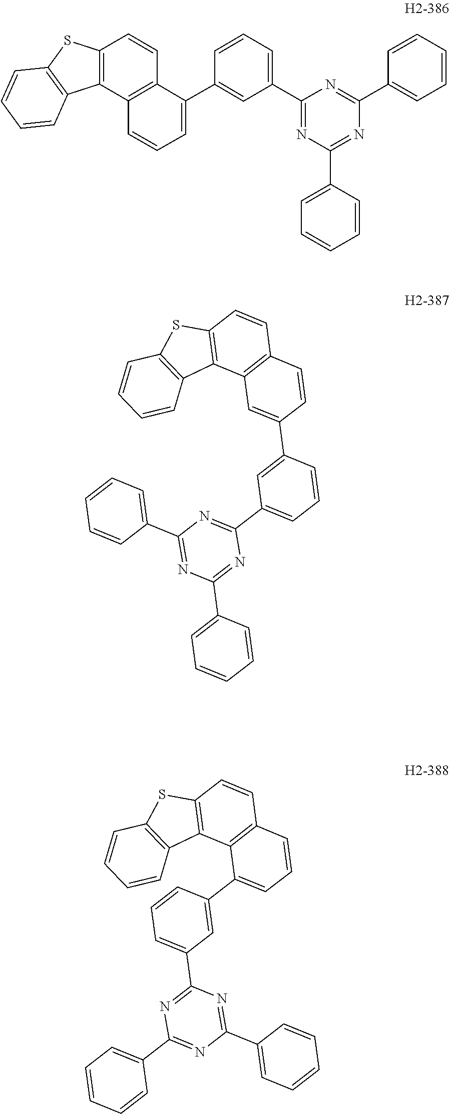 [SMILES: c1ccc(-c2nc(-c3ccccc3)nc(-c3cccc(-c4cccc5c4ccc4sc6ccccc6c45)c3)n2)cc1 ; c1ccc(-c2nc(-c3ccccc3)nc(-c3cccc(-c4ccc5ccc6sc7ccccc7c6c5c4)c3)n2)cc1 ; c1ccc(-c2nc(-c3ccccc3)nc(-c3cccc(-c4cccc5ccc6sc7ccccc7c6c45)c3)n2)cc1]

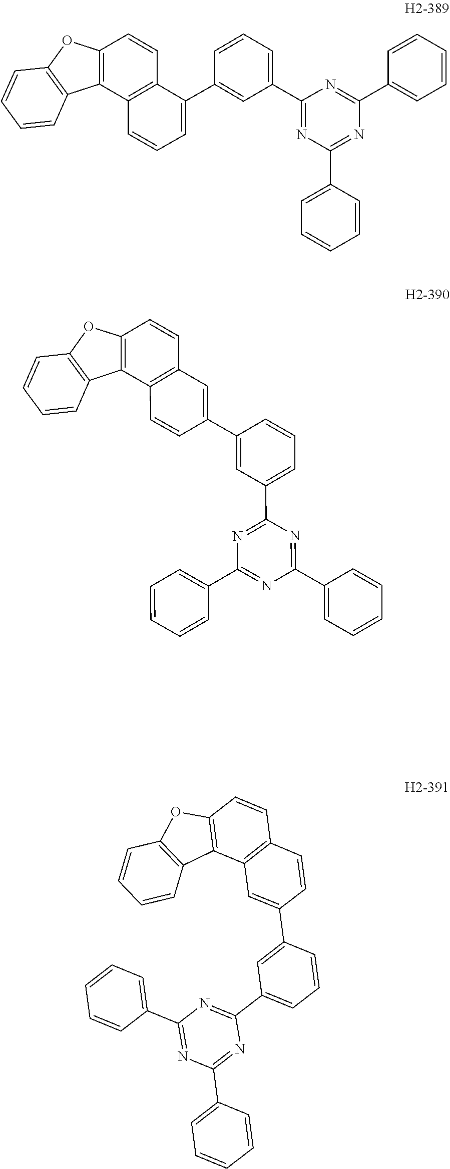 [SMILES: c1ccc(-c2nc(-c3ccccc3)nc(-c3cccc(-c4cccc5c4ccc4oc6ccccc6c45)c3)n2)cc1 ; c1ccc(-c2nc(-c3ccccc3)nc(-c3cccc(-c4ccc5c(ccc6oc7ccccc7c65)c4)c3)n2)cc1 ; c1ccc(-c2nc(-c3ccccc3)nc(-c3cccc(-c4ccc5ccc6oc7ccccc7c6c5c4)c3)n2)cc1]

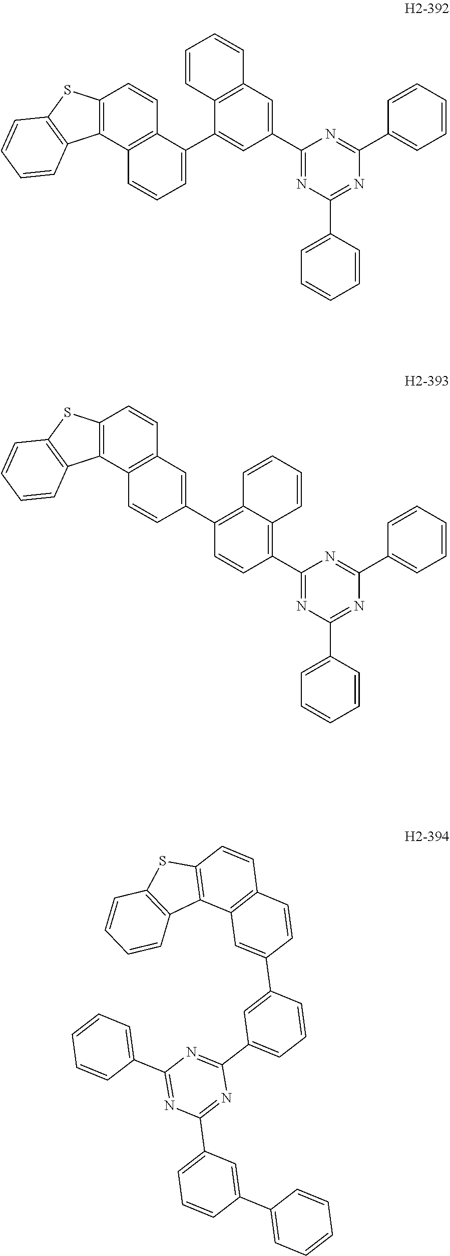 [SMILES: c1ccc(-c2nc(-c3ccccc3)nc(-c3cc(-c4cccc5c4ccc4sc6ccccc6c45)c4ccccc4c3)n2)cc1 ; c1ccc(-c2nc(-c3ccccc3)nc(-c3ccc(-c4ccc5c(ccc6sc7ccccc7c65)c4)c4ccccc34)n2)cc1 ; c1ccc(-c2cccc(-c3nc(-c4ccccc4)nc(-c4cccc(-c5ccc6ccc7sc8ccccc8c7c6c5)c4)n3)c2)cc1]

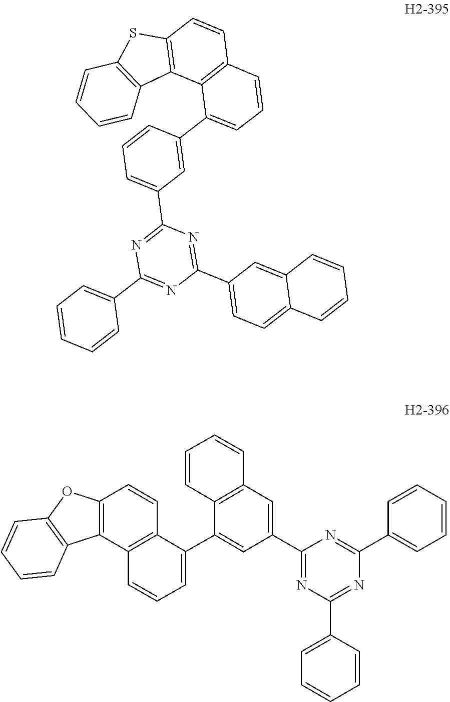 [SMILES: c1ccc(-c2nc(-c3cccc(-c4cccc5ccc6sc7ccccc7c6c45)c3)nc(-c3ccc4ccccc4c3)n2)cc1 ; c1ccc(-c2nc(-c3ccccc3)nc(-c3cc(-c4cccc5c4ccc4oc6ccccc6c45)c4ccccc4c3)n2)cc1]

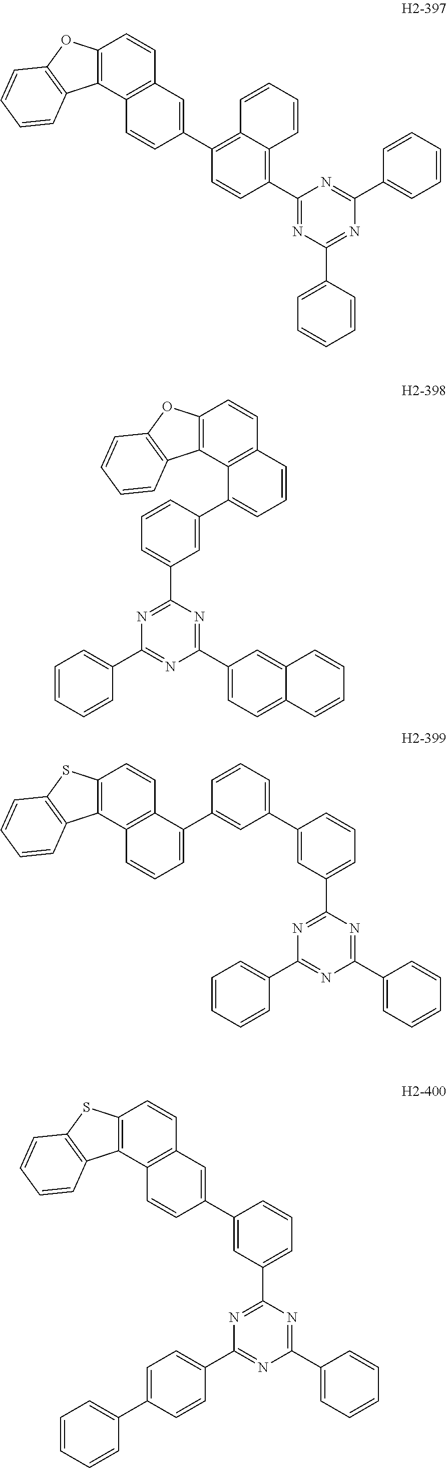 [SMILES: c1ccc(-c2nc(-c3ccccc3)nc(-c3ccc(-c4ccc5c(ccc6oc7ccccc7c65)c4)c4ccccc34)n2)cc1 ; c1ccc(-c2nc(-c3cccc(-c4cccc5ccc6oc7ccccc7c6c45)c3)nc(-c3ccc4ccccc4c3)n2)cc1 ; c1ccc(-c2nc(-c3ccccc3)nc(-c3cccc(-c4cccc(-c5cccc6c5ccc5sc7ccccc7c56)c4)c3)n2)cc1 ; c1ccc(-c2ccc(-c3nc(-c4ccccc4)nc(-c4cccc(-c5ccc6c(ccc7sc8ccccc8c76)c5)c4)n3)cc2)cc1]

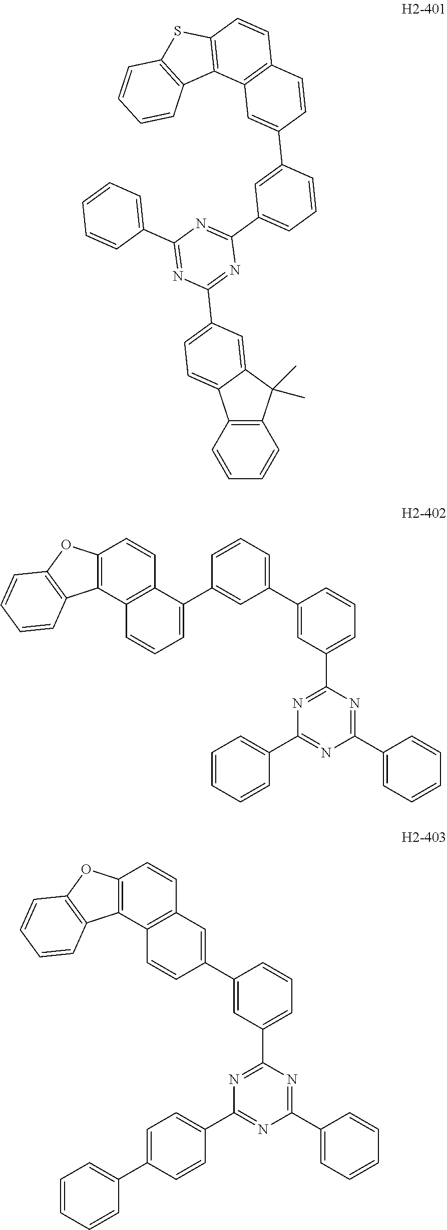 [SMILES: CC1(C)c2ccccc2-c2ccc(-c3nc(-c4ccccc4)nc(-c4cccc(-c5ccc6ccc7sc8ccccc8c7c6c5)c4)n3)cc21 ; c1ccc(-c2nc(-c3ccccc3)nc(-c3cccc(-c4cccc(-c5cccc6c5ccc5oc7ccccc7c56)c4)c3)n2)cc1 ; c1ccc(-c2ccc(-c3nc(-c4ccccc4)nc(-c4cccc(-c5ccc6c(ccc7oc8ccccc8c76)c5)c4)n3)cc2)cc1]

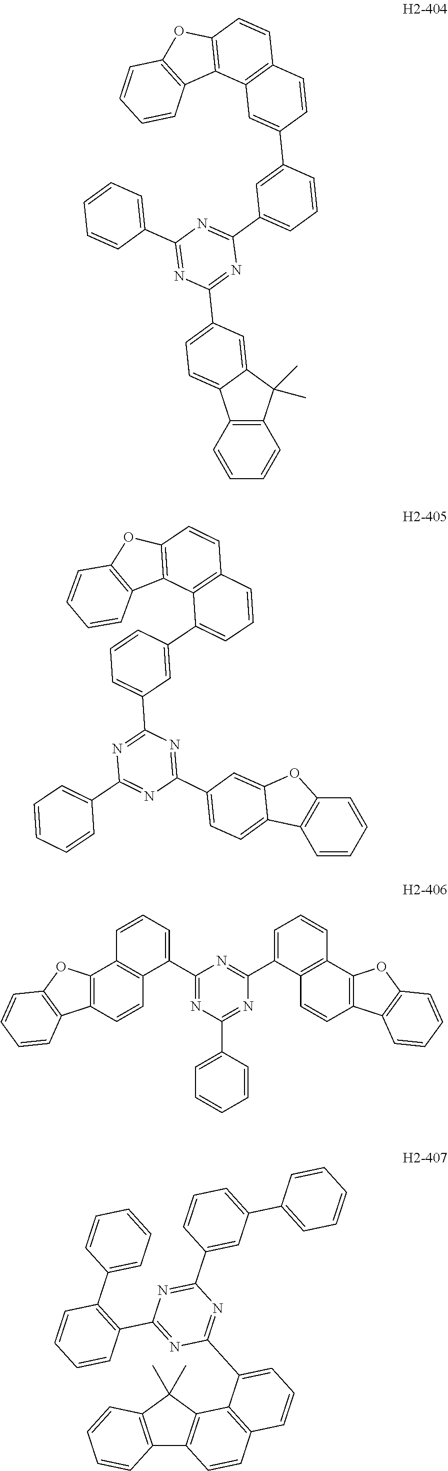 [SMILES: CC1(C)c2ccccc2-c2ccc(-c3nc(-c4ccccc4)nc(-c4cccc(-c5ccc6ccc7oc8ccccc8c7c6c5)c4)n3)cc21 ; c1ccc(-c2nc(-c3cccc(-c4cccc5ccc6oc7ccccc7c6c45)c3)nc(-c3ccc4c(c3)oc3ccccc34)n2)cc1 ; c1ccc(-c2nc(-c3cccc4c3ccc3c5ccccc5oc43)nc(-c3cccc4c3ccc3c5ccccc5oc43)n2)cc1 ; CC1(C)c2ccccc2-c2ccc3cccc(-c4nc(-c5cccc(-c6ccccc6)c5)nc(-c5ccccc5-c5ccccc5)n4)c3c21]

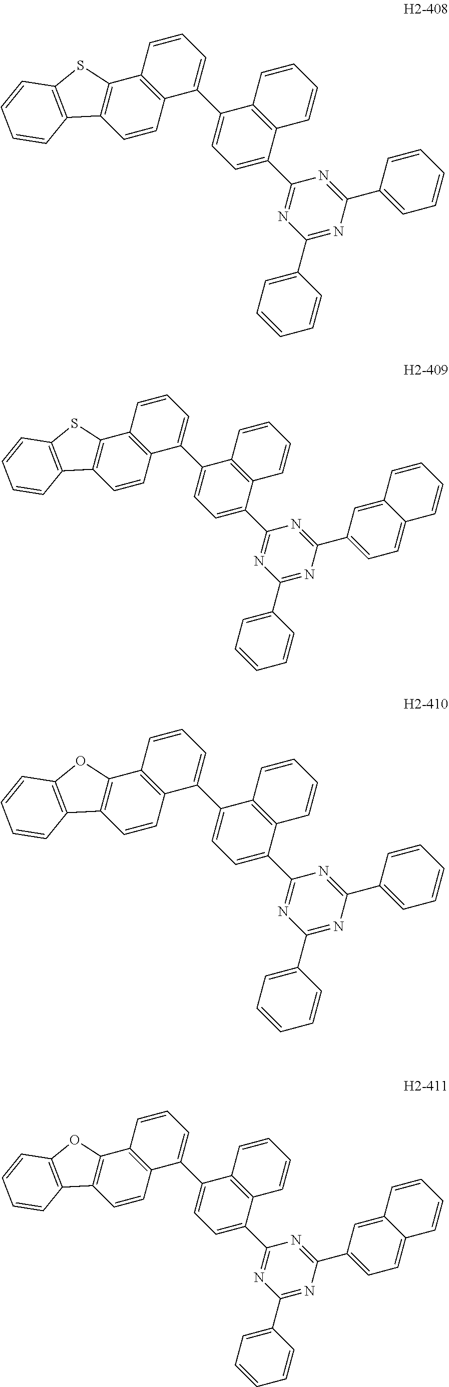 [SMILES: c1ccc(-c2nc(-c3ccccc3)nc(-c3ccc(-c4cccc5c4ccc4c6ccccc6sc54)c4ccccc34)n2)cc1 ; c1ccc(-c2nc(-c3ccc4ccccc4c3)nc(-c3ccc(-c4cccc5c4ccc4c6ccccc6sc54)c4ccccc34)n2)cc1 ; c1ccc(-c2nc(-c3ccccc3)nc(-c3ccc(-c4cccc5c4ccc4c6ccccc6oc54)c4ccccc34)n2)cc1 ; c1ccc(-c2nc(-c3ccc4ccccc4c3)nc(-c3ccc(-c4cccc5c4ccc4c6ccccc6oc54)c4ccccc34)n2)cc1]

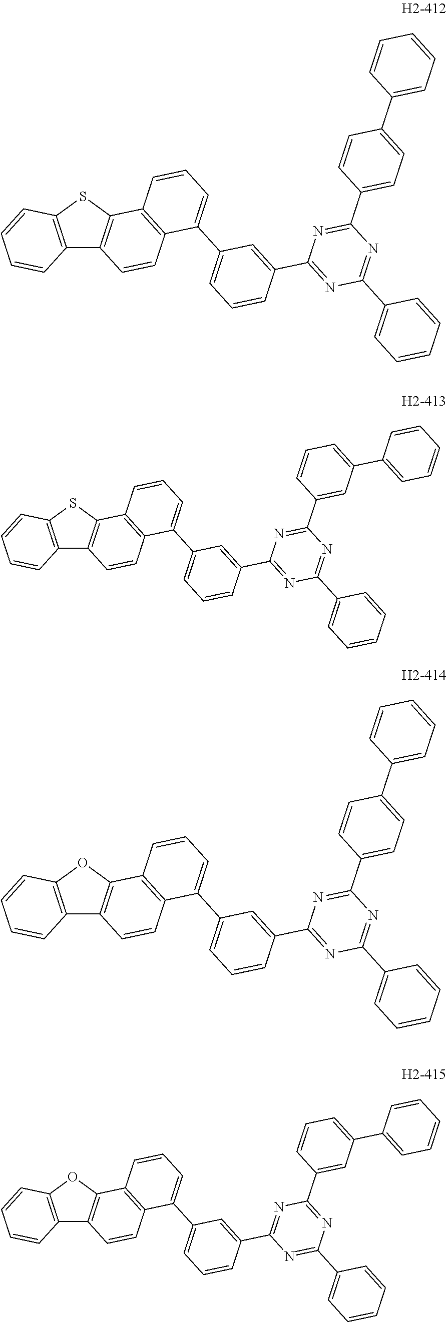 [SMILES: c1ccc(-c2ccc(-c3nc(-c4ccccc4)nc(-c4cccc(-c5cccc6c5ccc5c7ccccc7sc65)c4)n3)cc2)cc1 ; c1ccc(-c2cccc(-c3nc(-c4ccccc4)nc(-c4cccc(-c5cccc6c5ccc5c7ccccc7sc65)c4)n3)c2)cc1 ; c1ccc(-c2ccc(-c3nc(-c4ccccc4)nc(-c4cccc(-c5cccc6c5ccc5c7ccccc7oc65)c4)n3)cc2)cc1 ; c1ccc(-c2cccc(-c3nc(-c4ccccc4)nc(-c4cccc(-c5cccc6c5ccc5c7ccccc7oc65)c4)n3)c2)cc1]

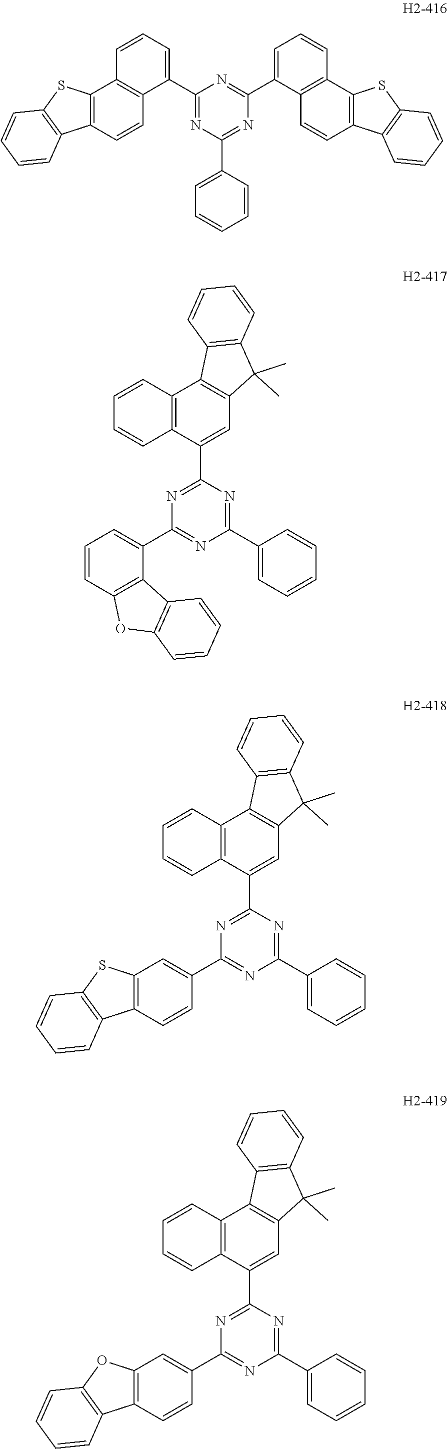 [SMILES: c1ccc(-c2nc(-c3cccc4c3ccc3c5ccccc5sc43)nc(-c3cccc4c3ccc3c5ccccc5sc43)n2)cc1 ; CC1(C)c2ccccc2-c2c1cc(-c1nc(-c3ccccc3)nc(-c3cccc4oc5ccccc5c34)n1)c1ccccc21 ; CC1(C)c2ccccc2-c2c1cc(-c1nc(-c3ccccc3)nc(-c3ccc4c(c3)sc3ccccc34)n1)c1ccccc21 ; CC1(C)c2ccccc2-c2c1cc(-c1nc(-c3ccccc3)nc(-c3ccc4c(c3)oc3ccccc34)n1)c1ccccc21]

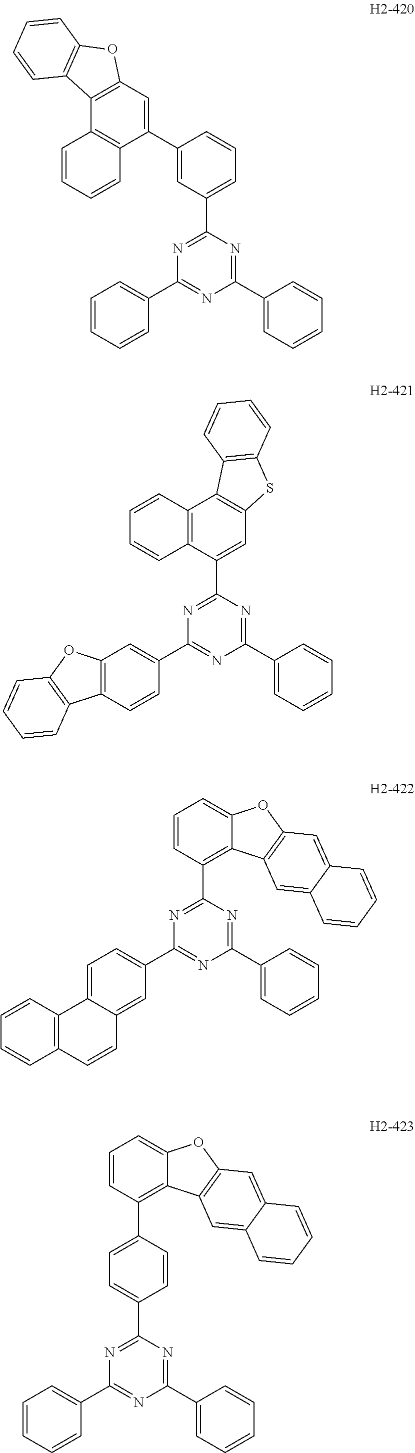 [SMILES: c1ccc(-c2nc(-c3ccccc3)nc(-c3cccc(-c4cc5oc6ccccc6c5c5ccccc45)c3)n2)cc1 ; c1ccc(-c2nc(-c3ccc4c(c3)oc3ccccc34)nc(-c3cc4sc5ccccc5c4c4ccccc34)n2)cc1 ; c1ccc(-c2nc(-c3ccc4c(ccc5ccccc54)c3)nc(-c3cccc4oc5cc6ccccc6cc5c34)n2)cc1 ; c1ccc(-c2nc(-c3ccccc3)nc(-c3ccc(-c4cccc5oc6cc7ccccc7cc6c45)cc3)n2)cc1]

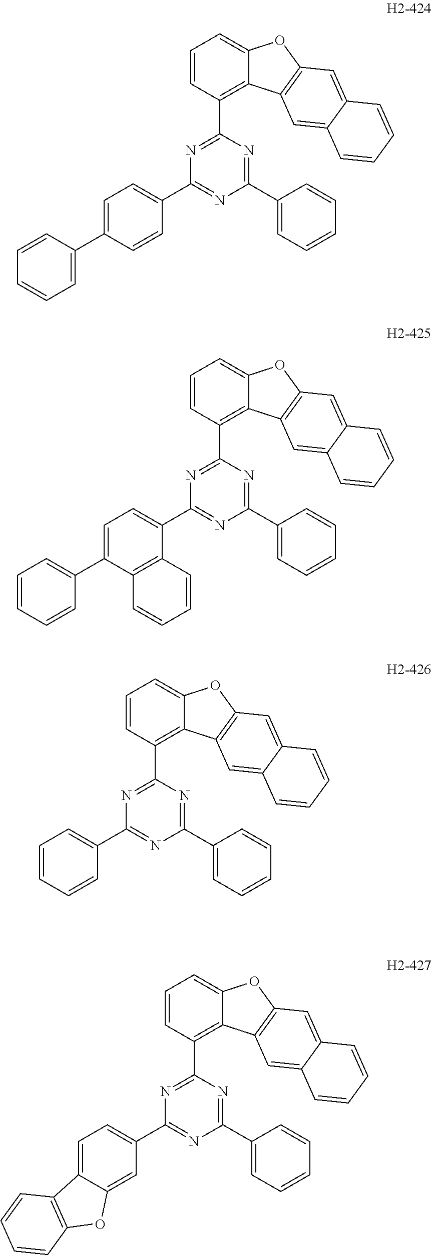 [SMILES: c1ccc(-c2ccc(-c3nc(-c4ccccc4)nc(-c4cccc5oc6cc7ccccc7cc6c45)n3)cc2)cc1 ; c1ccc(-c2nc(-c3ccc(-c4ccccc4)c4ccccc34)nc(-c3cccc4oc5cc6ccccc6cc5c34)n2)cc1 ; c1ccc(-c2nc(-c3ccccc3)nc(-c3cccc4oc5cc6ccccc6cc5c34)n2)cc1 ; c1ccc(-c2nc(-c3ccc4c(c3)oc3ccccc34)nc(-c3cccc4oc5cc6ccccc6cc5c34)n2)cc1]

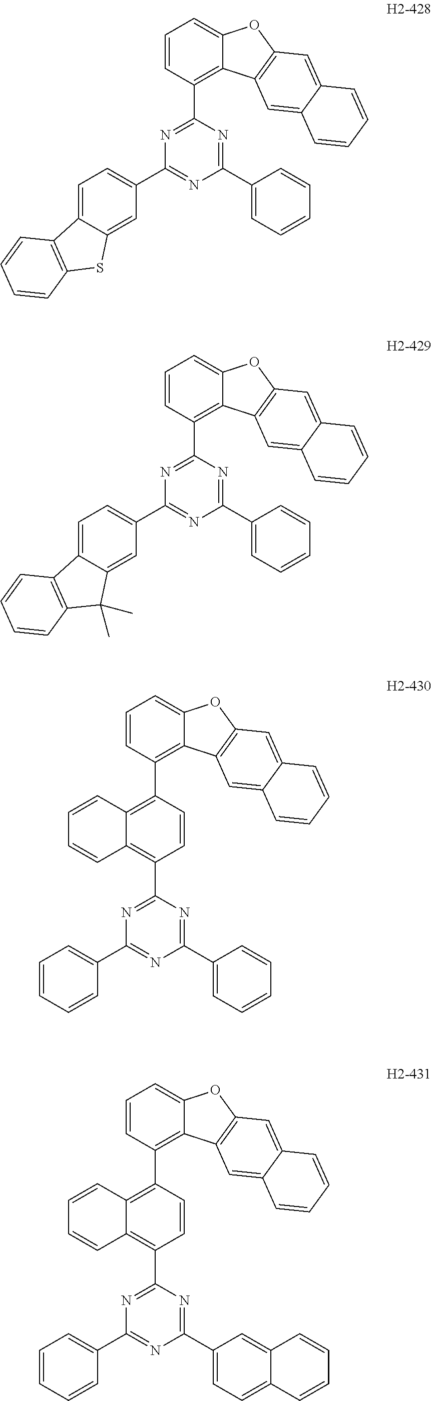 [SMILES: c1ccc(-c2nc(-c3ccc4c(c3)sc3ccccc34)nc(-c3cccc4oc5cc6ccccc6cc5c34)n2)cc1 ; CC1(C)c2ccccc2-c2ccc(-c3nc(-c4ccccc4)nc(-c4cccc5oc6cc7ccccc7cc6c45)n3)cc21 ; c1ccc(-c2nc(-c3ccccc3)nc(-c3ccc(-c4cccc5oc6cc7ccccc7cc6c45)c4ccccc34)n2)cc1 ; c1ccc(-c2nc(-c3ccc4ccccc4c3)nc(-c3ccc(-c4cccc5oc6cc7ccccc7cc6c45)c4ccccc34)n2)cc1]

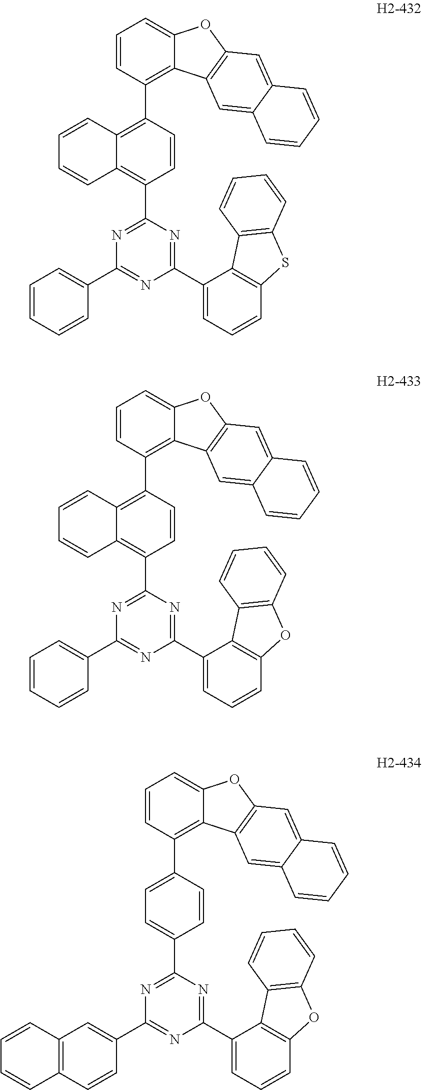 [SMILES: c1ccc(-c2nc(-c3ccc(-c4cccc5oc6cc7ccccc7cc6c45)c4ccccc34)nc(-c3cccc4sc5ccccc5c34)n2)cc1 ; c1ccc(-c2nc(-c3ccc(-c4cccc5oc6cc7ccccc7cc6c45)c4ccccc34)nc(-c3cccc4oc5ccccc5c34)n2)cc1 ; c1ccc2cc(-c3nc(-c4ccc(-c5cccc6oc7cc8ccccc8cc7c56)cc4)nc(-c4cccc5oc6ccccc6c45)n3)ccc2c1]

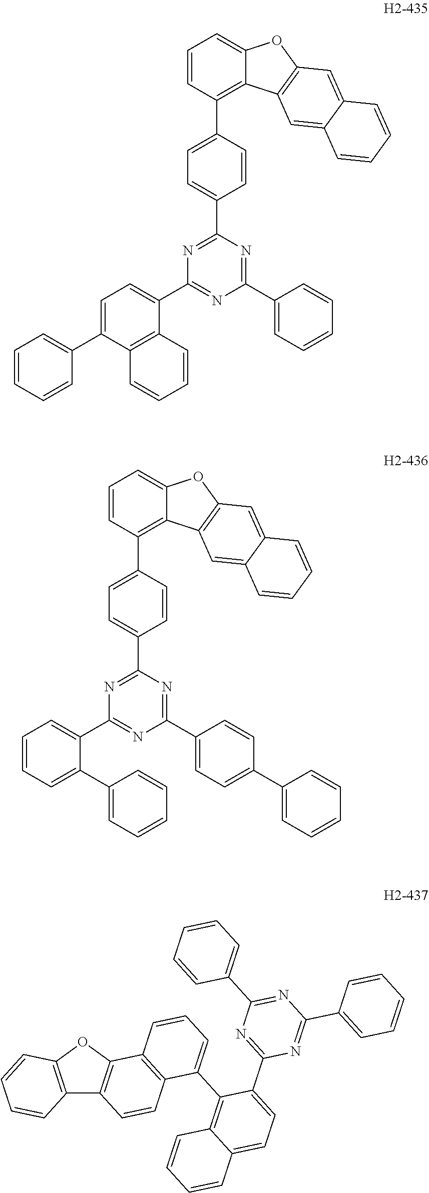 [SMILES: c1ccc(-c2nc(-c3ccc(-c4cccc5oc6cc7ccccc7cc6c45)cc3)nc(-c3ccc(-c4ccccc4)c4ccccc34)n2)cc1 ; c1ccc(-c2ccc(-c3nc(-c4ccc(-c5cccc6oc7cc8ccccc8cc7c56)cc4)nc(-c4ccccc4-c4ccccc4)n3)cc2)cc1 ; c1ccc(-c2nc(-c3ccccc3)nc(-c3ccc4ccccc4c3-c3cccc4c3ccc3c5ccccc5oc43)n2)cc1]

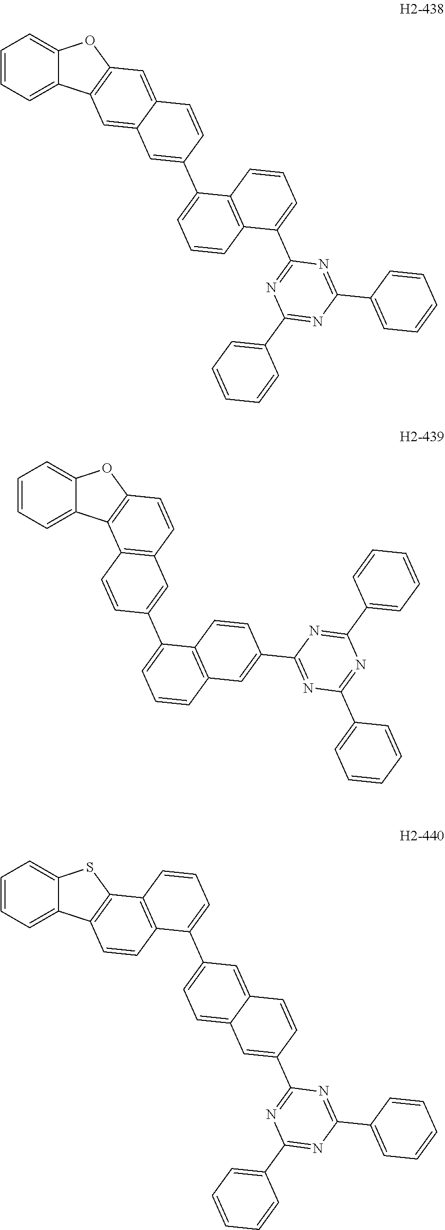 [SMILES: c1ccc(-c2nc(-c3ccccc3)nc(-c3cccc4c(-c5ccc6cc7oc8ccccc8c7cc6c5)cccc34)n2)cc1 ; c1ccc(-c2nc(-c3ccccc3)nc(-c3ccc4c(-c5ccc6c(ccc7oc8ccccc8c76)c5)cccc4c3)n2)cc1 ; c1ccc(-c2nc(-c3ccccc3)nc(-c3ccc4cc(-c5cccc6c5ccc5c7ccccc7sc65)ccc4c3)n2)cc1]

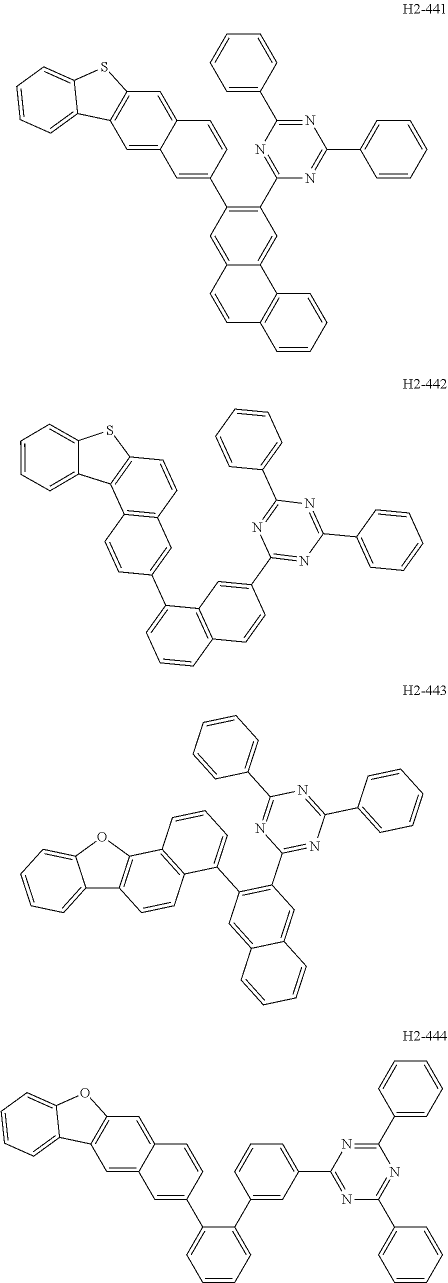 [SMILES: c1ccc(-c2nc(-c3ccccc3)nc(-c3cc4c(ccc5ccccc54)cc3-c3ccc4cc5sc6ccccc6c5cc4c3)n2)cc1 ; c1ccc(-c2nc(-c3ccccc3)nc(-c3ccc4cccc(-c5ccc6c(ccc7sc8ccccc8c76)c5)c4c3)n2)cc1 ; c1ccc(-c2nc(-c3ccccc3)nc(-c3cc4ccccc4cc3-c3cccc4c3ccc3c5ccccc5oc43)n2)cc1 ; c1ccc(-c2nc(-c3ccccc3)nc(-c3cccc(-c4ccccc4-c4ccc5cc6oc7ccccc7c6cc5c4)c3)n2)cc1]

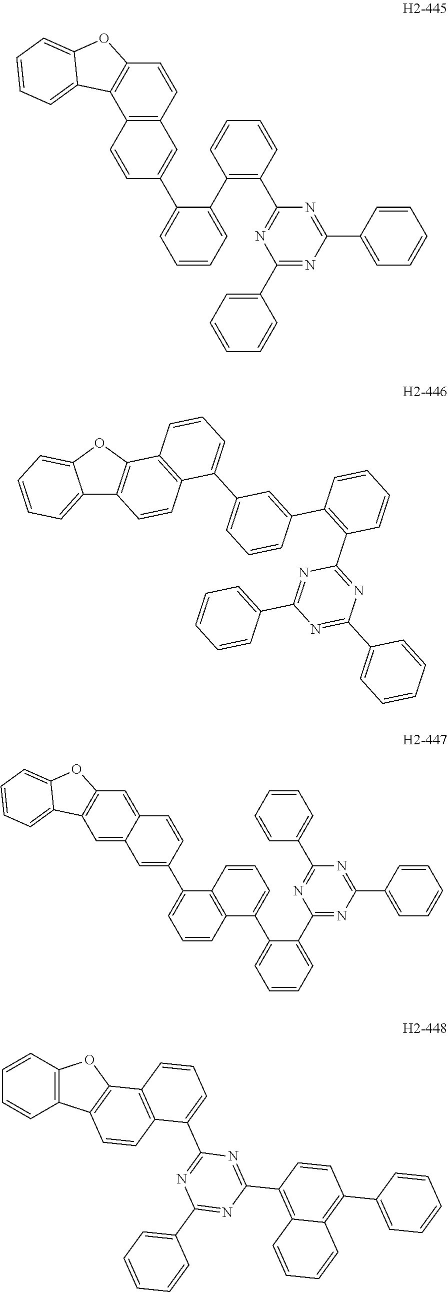 [SMILES: c1ccc(-c2nc(-c3ccccc3)nc(-c3ccccc3-c3ccccc3-c3ccc4c(ccc5oc6ccccc6c54)c3)n2)cc1 ; c1ccc(-c2nc(-c3ccccc3)nc(-c3ccccc3-c3cccc(-c4cccc5c4ccc4c6ccccc6oc54)c3)n2)cc1 ; c1ccc(-c2nc(-c3ccccc3)nc(-c3ccccc3-c3cccc4c(-c5ccc6cc7oc8ccccc8c7cc6c5)cccc34)n2)cc1 ; c1ccc(-c2nc(-c3ccc(-c4ccccc4)c4ccccc34)nc(-c3cccc4c3ccc3c5ccccc5oc43)n2)cc1]

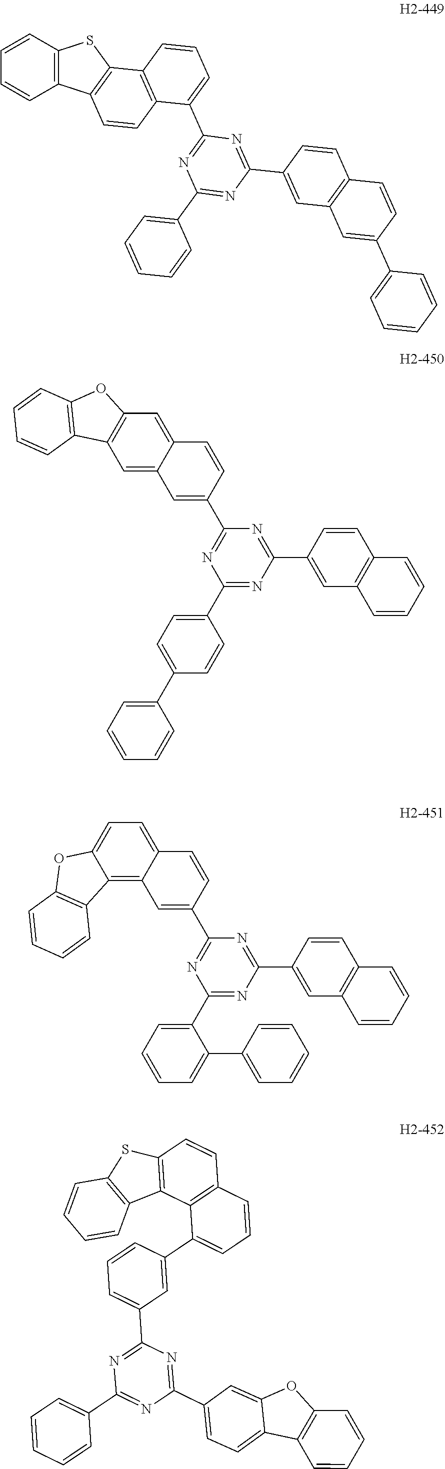 [SMILES: c1ccc(-c2ccc3ccc(-c4nc(-c5ccccc5)nc(-c5cccc6c5ccc5c7ccccc7sc65)n4)cc3c2)cc1 ; c1ccc(-c2ccc(-c3nc(-c4ccc5ccccc5c4)nc(-c4ccc5cc6oc7ccccc7c6cc5c4)n3)cc2)cc1 ; c1ccc(-c2ccccc2-c2nc(-c3ccc4ccccc4c3)nc(-c3ccc4ccc5oc6ccccc6c5c4c3)n2)cc1 ; c1ccc(-c2nc(-c3cccc(-c4cccc5ccc6sc7ccccc7c6c45)c3)nc(-c3ccc4c(c3)oc3ccccc34)n2)cc1]

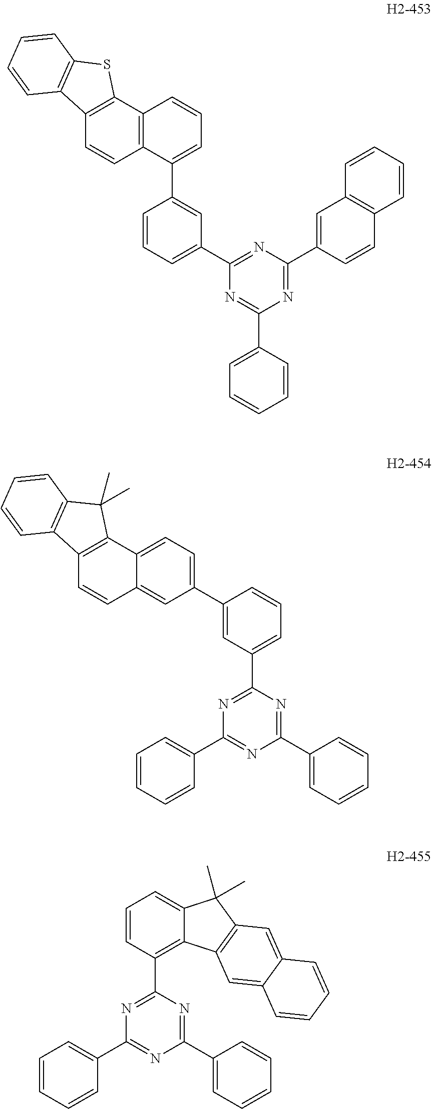 [SMILES: c1ccc(-c2nc(-c3cccc(-c4cccc5c4ccc4c6ccccc6sc54)c3)nc(-c3ccc4ccccc4c3)n2)cc1 ; CC1(C)c2ccccc2-c2ccc3cc(-c4cccc(-c5nc(-c6ccccc6)nc(-c6ccccc6)n5)c4)ccc3c21 ; CC1(C)c2cc3ccccc3cc2-c2c(-c3nc(-c4ccccc4)nc(-c4ccccc4)n3)cccc21]

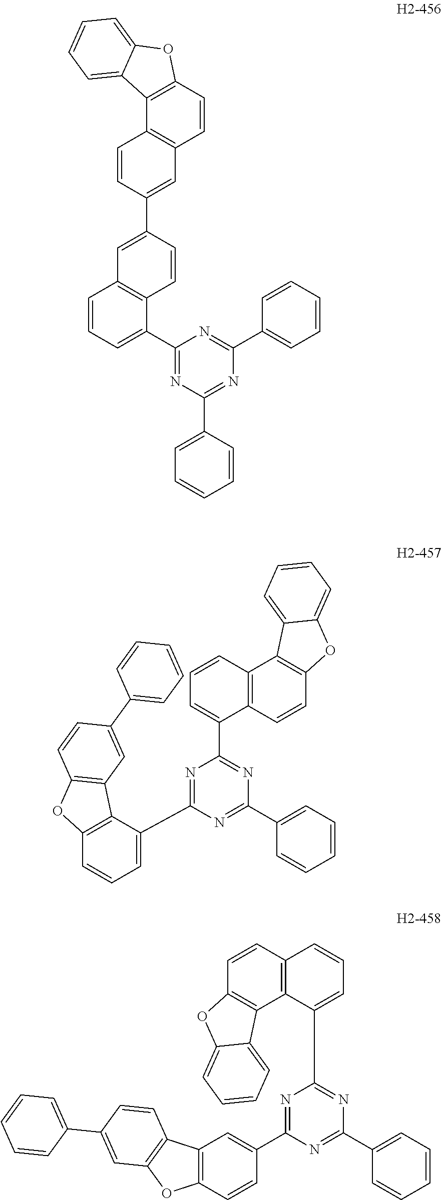 [SMILES: c1ccc(-c2nc(-c3ccccc3)nc(-c3cccc4cc(-c5ccc6c(ccc7oc8ccccc8c76)c5)ccc34)n2)cc1 ; c1ccc(-c2ccc3oc4cccc(-c5nc(-c6ccccc6)nc(-c6cccc7c6ccc6oc8ccccc8c67)n5)c4c3c2)cc1 ; c1ccc(-c2ccc3c(c2)oc2ccc(-c4nc(-c5ccccc5)nc(-c5cccc6ccc7oc8ccccc8c7c56)n4)cc23)cc1]

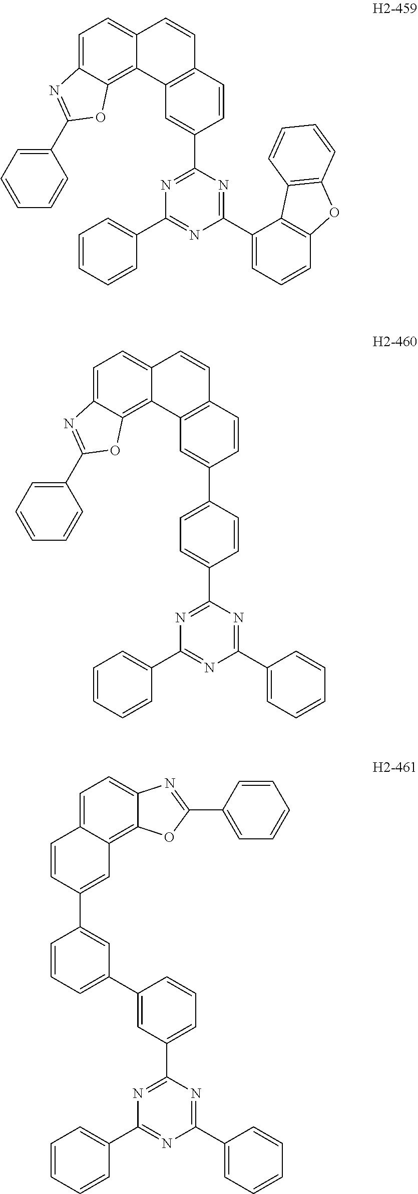 [SMILES: c1ccc(-c2nc(-c3ccc4ccc5ccc6nc(-c7ccccc7)oc6c5c4c3)nc(-c3cccc4oc5ccccc5c34)n2)cc1 ; c1ccc(-c2nc(-c3ccccc3)nc(-c3ccc(-c4ccc5ccc6ccc7nc(-c8ccccc8)oc7c6c5c4)cc3)n2)cc1 ; c1ccc(-c2nc(-c3ccccc3)nc(-c3cccc(-c4cccc(-c5ccc6ccc7nc(-c8ccccc8)oc7c6c5)c4)c3)n2)cc1]

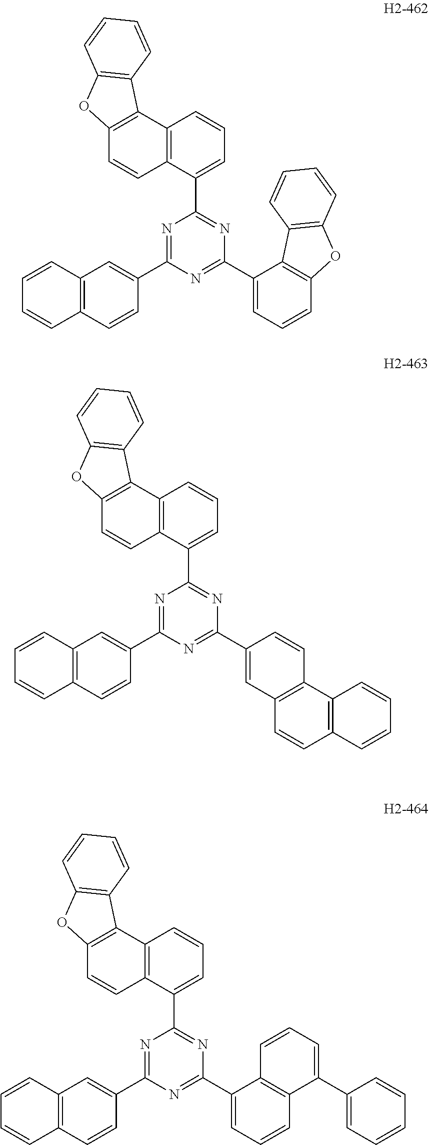 [SMILES: c1ccc2cc(-c3nc(-c4cccc5c4ccc4oc6ccccc6c45)nc(-c4cccc5oc6ccccc6c45)n3)ccc2c1 ; c1ccc2cc(-c3nc(-c4ccc5c(ccc6ccccc65)c4)nc(-c4cccc5c4ccc4oc6ccccc6c45)n3)ccc2c1 ; c1ccc(-c2cccc3c(-c4nc(-c5ccc6ccccc6c5)nc(-c5cccc6c5ccc5oc7ccccc7c56)n4)cccc23)cc1]

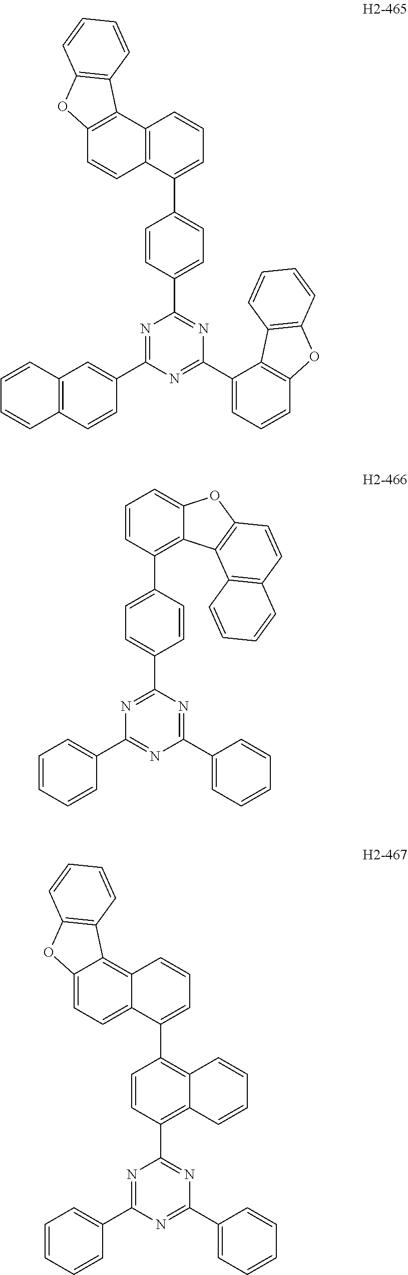 [SMILES: c1ccc2cc(-c3nc(-c4ccc(-c5cccc6c5ccc5oc7ccccc7c56)cc4)nc(-c4cccc5oc6ccccc6c45)n3)ccc2c1 ; c1ccc(-c2nc(-c3ccccc3)nc(-c3ccc(-c4cccc5oc6ccc7ccccc7c6c45)cc3)n2)cc1 ; c1ccc(-c2nc(-c3ccccc3)nc(-c3ccc(-c4cccc5c4ccc4oc6ccccc6c45)c4ccccc34)n2)cc1]

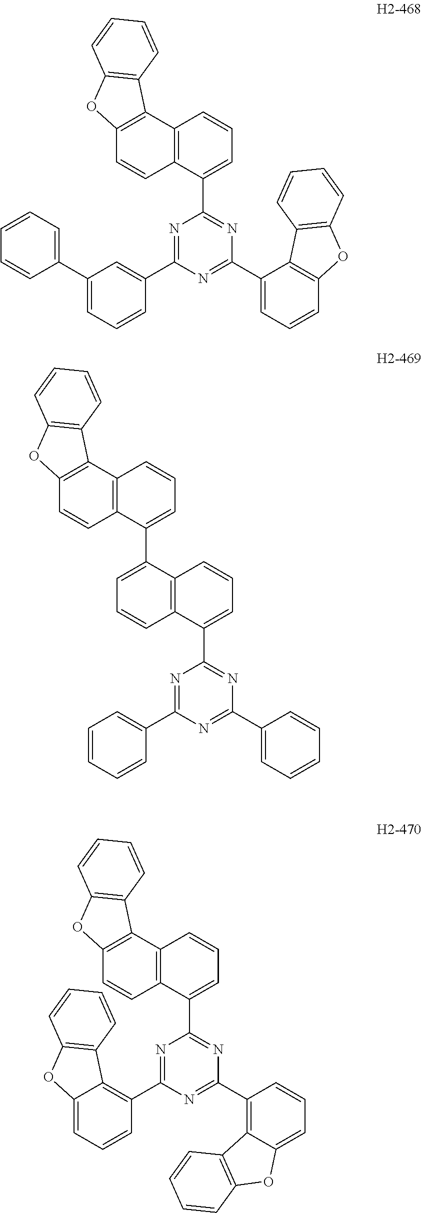 [SMILES: c1ccc(-c2cccc(-c3nc(-c4cccc5c4ccc4oc6ccccc6c45)nc(-c4cccc5oc6ccccc6c45)n3)c2)cc1 ; c1ccc(-c2nc(-c3ccccc3)nc(-c3cccc4c(-c5cccc6c5ccc5oc7ccccc7c56)cccc34)n2)cc1 ; c1ccc2c(c1)oc1cccc(-c3nc(-c4cccc5c4ccc4oc6ccccc6c45)nc(-c4cccc5oc6ccccc6c45)n3)c12]

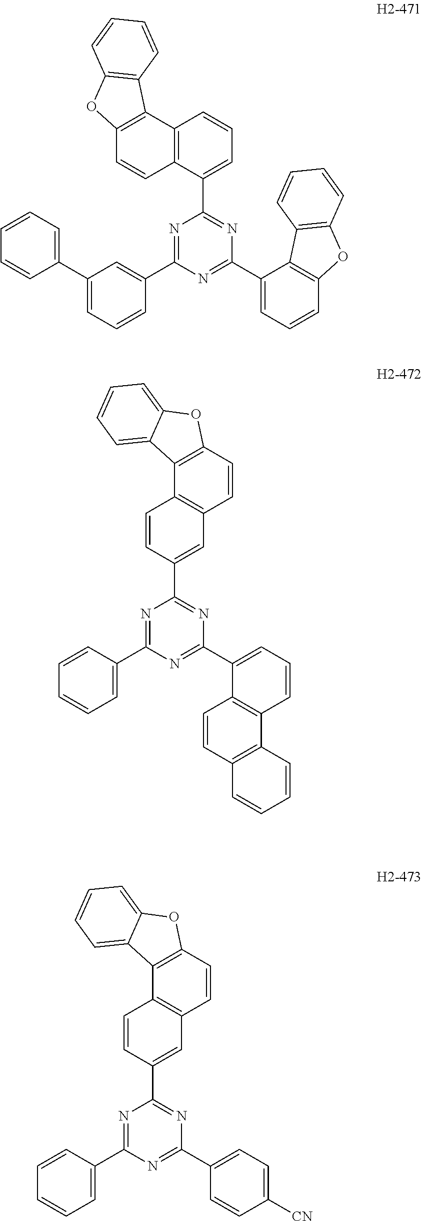 [SMILES: c1ccc(-c2cccc(-c3nc(-c4cccc5c4ccc4oc6ccccc6c45)nc(-c4cccc5oc6ccccc6c45)n3)c2)cc1 ; c1ccc(-c2nc(-c3ccc4c(ccc5oc6ccccc6c54)c3)nc(-c3cccc4c3ccc3ccccc34)n2)cc1 ; N#Cc1ccc(-c2nc(-c3ccccc3)nc(-c3ccc4c(ccc5oc6ccccc6c54)c3)n2)cc1]

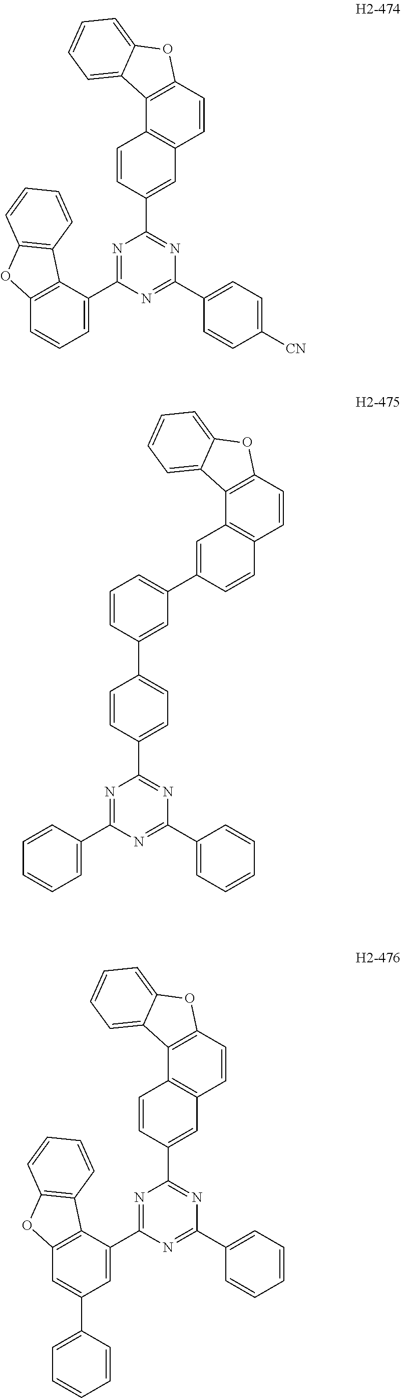 [SMILES: N#Cc1ccc(-c2nc(-c3ccc4c(ccc5oc6ccccc6c54)c3)nc(-c3cccc4oc5ccccc5c34)n2)cc1 ; c1ccc(-c2nc(-c3ccccc3)nc(-c3ccc(-c4cccc(-c5ccc6ccc7oc8ccccc8c7c6c5)c4)cc3)n2)cc1 ; c1ccc(-c2cc(-c3nc(-c4ccccc4)nc(-c4ccc5c(ccc6oc7ccccc7c65)c4)n3)c3c(c2)oc2ccccc23)cc1]

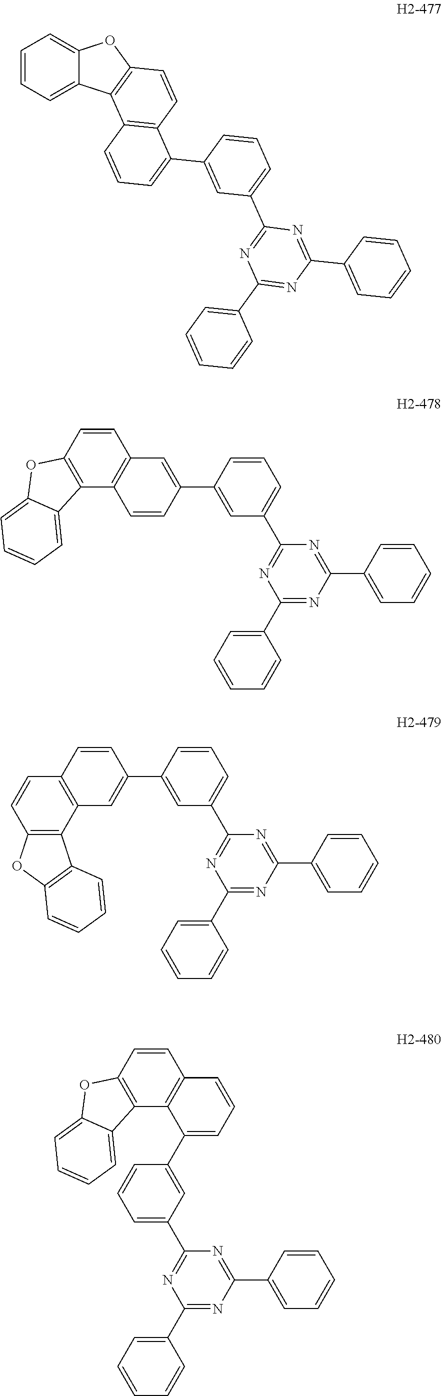 [SMILES: c1ccc(-c2nc(-c3ccccc3)nc(-c3cccc(-c4cccc5c4ccc4oc6ccccc6c45)c3)n2)cc1 ; c1ccc(-c2nc(-c3ccccc3)nc(-c3cccc(-c4ccc5c(ccc6oc7ccccc7c65)c4)c3)n2)cc1 ; c1ccc(-c2nc(-c3ccccc3)nc(-c3cccc(-c4ccc5ccc6oc7ccccc7c6c5c4)c3)n2)cc1 ; c1ccc(-c2nc(-c3ccccc3)nc(-c3cccc(-c4cccc5ccc6oc7ccccc7c6c45)c3)n2)cc1]

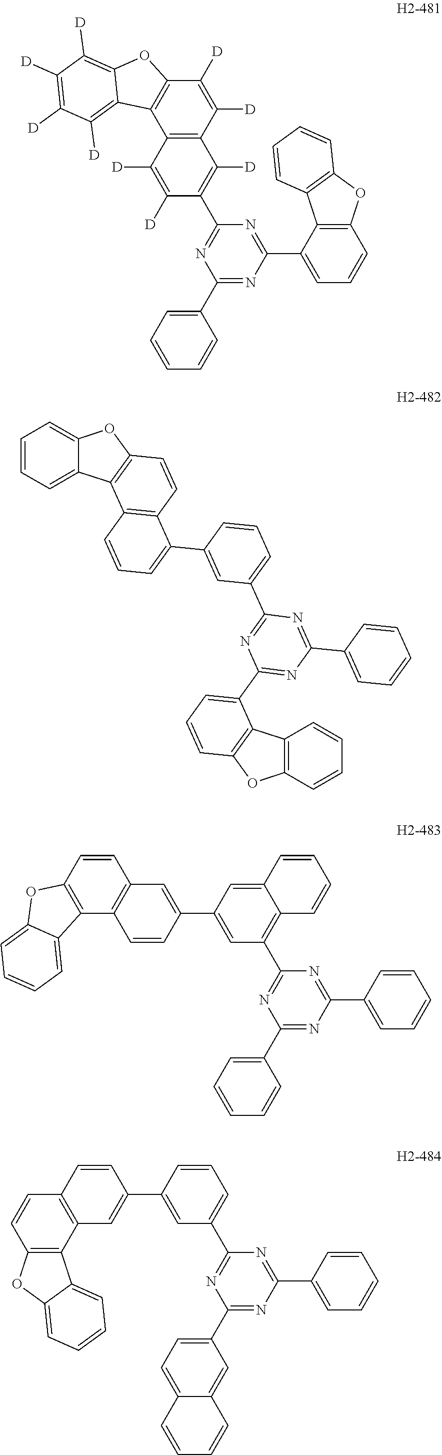 [SMILES: [2H]c1c([2H])c([2H])c2c(oc3c([2H])c([2H])c4c([2H])c(-c5nc(-c6ccccc6)nc(-c6cccc7oc8ccccc8c67)n5)c([2H])c([2H])c4c32)c1[2H] ; c1ccc(-c2nc(-c3cccc(-c4cccc5c4ccc4oc6ccccc6c45)c3)nc(-c3cccc4oc5ccccc5c34)n2)cc1 ; c1ccc(-c2nc(-c3ccccc3)nc(-c3cc(-c4ccc5c(ccc6oc7ccccc7c65)c4)cc4ccccc34)n2)cc1 ; c1ccc(-c2nc(-c3cccc(-c4ccc5ccc6oc7ccccc7c6c5c4)c3)nc(-c3ccc4ccccc4c3)n2)cc1]

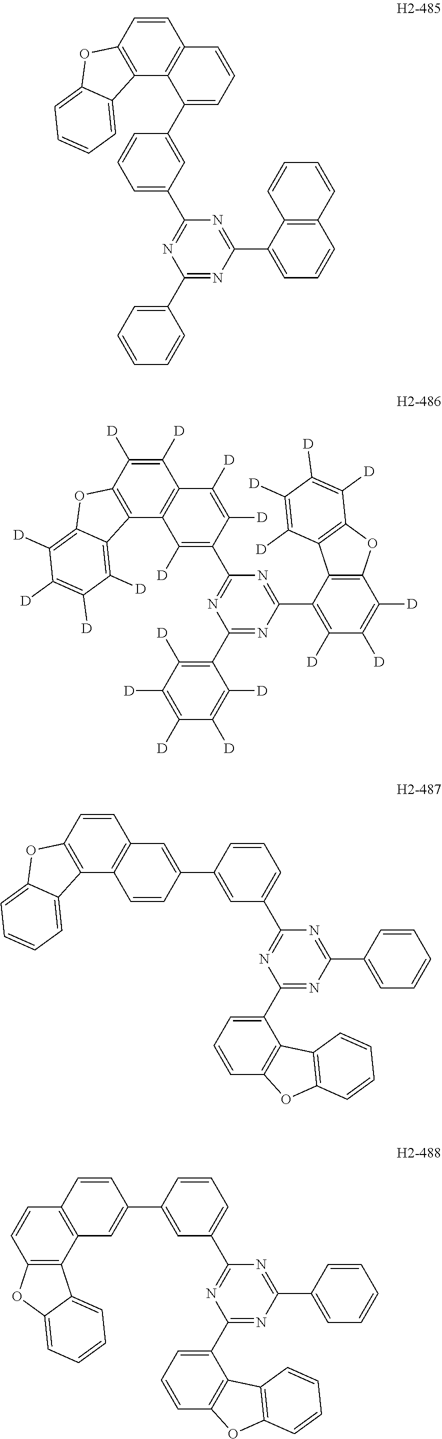 [SMILES: c1ccc(-c2nc(-c3cccc(-c4cccc5ccc6oc7ccccc7c6c45)c3)nc(-c3cccc4ccccc34)n2)cc1 ; [2H]c1c([2H])c([2H])c(-c2nc(-c3c([2H])c([2H])c4c([2H])c([2H])c5oc6c([2H])c([2H])c([2H])c([2H])c6c5c4c3[2H])nc(-c3c([2H])c([2H])c([2H])c4oc5c([2H])c([2H])c([2H])c([2H])c5c34)n2)c([2H])c1[2H] ; c1ccc(-c2nc(-c3cccc(-c4ccc5c(ccc6oc7ccccc7c65)c4)c3)nc(-c3cccc4oc5ccccc5c34)n2)cc1 ; c1ccc(-c2nc(-c3cccc(-c4ccc5ccc6oc7ccccc7c6c5c4)c3)nc(-c3cccc4oc5ccccc5c34)n2)cc1]

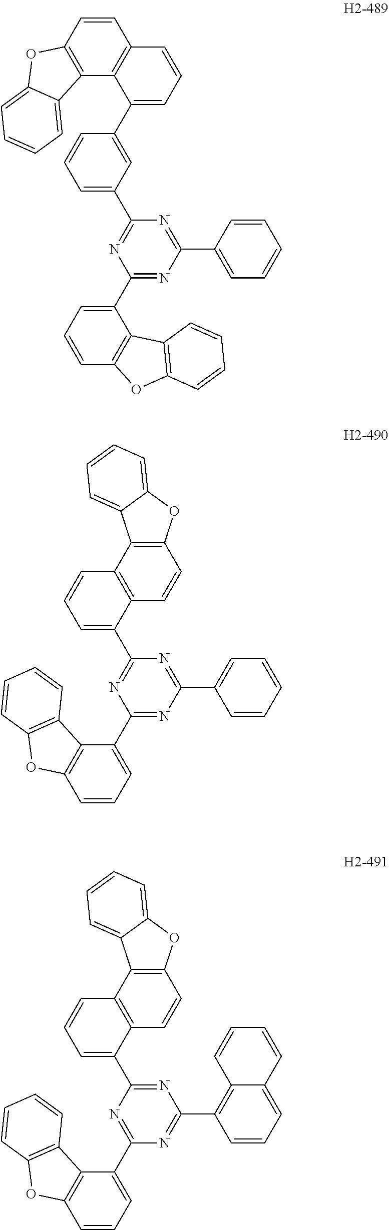 [SMILES: c1ccc(-c2nc(-c3cccc(-c4cccc5ccc6oc7ccccc7c6c45)c3)nc(-c3cccc4oc5ccccc5c34)n2)cc1 ; c1ccc(-c2nc(-c3cccc4c3ccc3oc5ccccc5c34)nc(-c3cccc4oc5ccccc5c34)n2)cc1 ; c1ccc2c(-c3nc(-c4cccc5c4ccc4oc6ccccc6c45)nc(-c4cccc5oc6ccccc6c45)n3)cccc2c1]

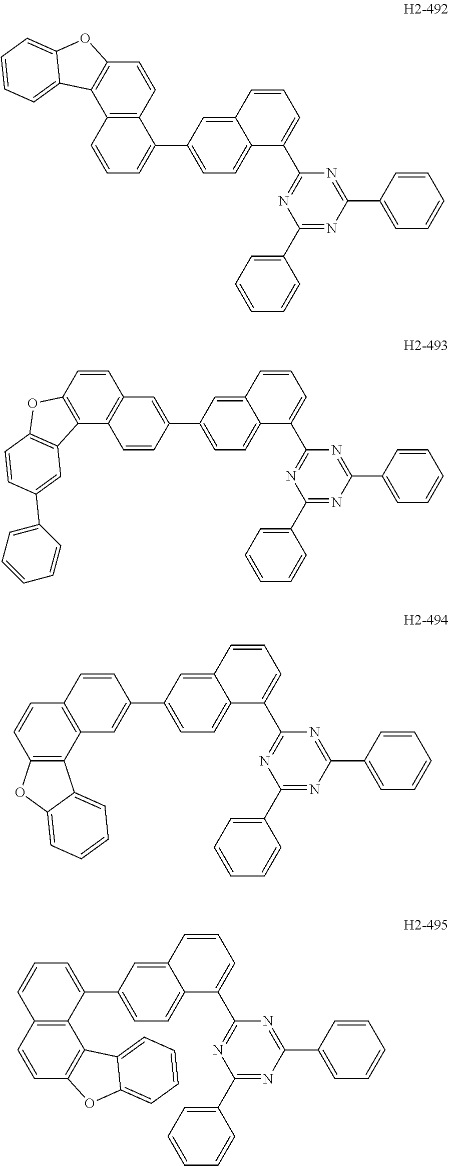 [SMILES: c1ccc(-c2nc(-c3ccccc3)nc(-c3cccc4cc(-c5cccc6c5ccc5oc7ccccc7c56)ccc34)n2)cc1 ; c1ccc(-c2ccc3oc4ccc5cc(-c6ccc7c(-c8nc(-c9ccccc9)nc(-c9ccccc9)n8)cccc7c6)ccc5c4c3c2)cc1 ; c1ccc(-c2nc(-c3ccccc3)nc(-c3cccc4cc(-c5ccc6ccc7oc8ccccc8c7c6c5)ccc34)n2)cc1 ; c1ccc(-c2nc(-c3ccccc3)nc(-c3cccc4cc(-c5cccc6ccc7oc8ccccc8c7c56)ccc34)n2)cc1]

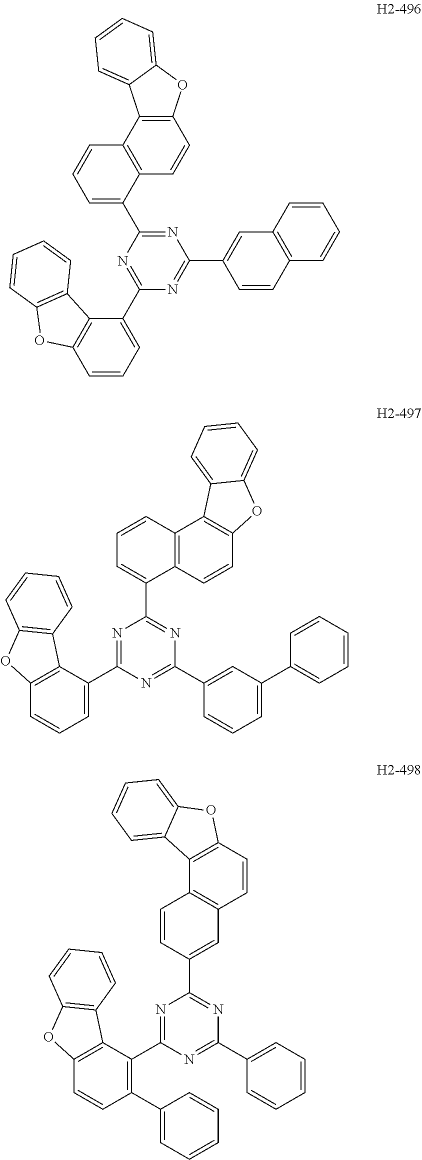 [SMILES: c1ccc2cc(-c3nc(-c4cccc5c4ccc4oc6ccccc6c45)nc(-c4cccc5oc6ccccc6c45)n3)ccc2c1 ; c1ccc(-c2cccc(-c3nc(-c4cccc5c4ccc4oc6ccccc6c45)nc(-c4cccc5oc6ccccc6c45)n3)c2)cc1 ; c1ccc(-c2nc(-c3ccc4c(ccc5oc6ccccc6c54)c3)nc(-c3c(-c4ccccc4)ccc4oc5ccccc5c34)n2)cc1]

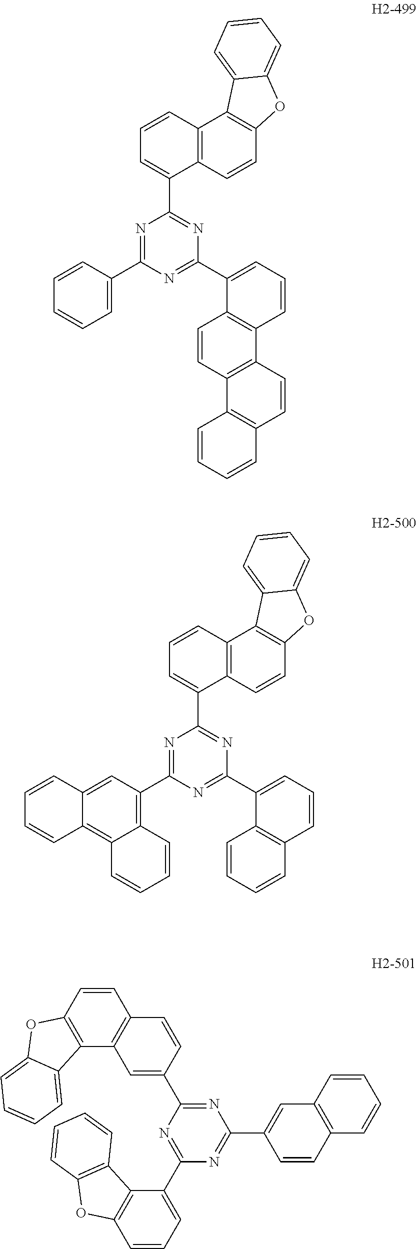 [SMILES: c1ccc(-c2nc(-c3cccc4c3ccc3c5ccccc5ccc43)nc(-c3cccc4c3ccc3oc5ccccc5c34)n2)cc1 ; c1ccc2c(-c3nc(-c4cc5ccccc5c5ccccc45)nc(-c4cccc5c4ccc4oc6ccccc6c45)n3)cccc2c1 ; c1ccc2cc(-c3nc(-c4ccc5ccc6oc7ccccc7c6c5c4)nc(-c4cccc5oc6ccccc6c45)n3)ccc2c1]

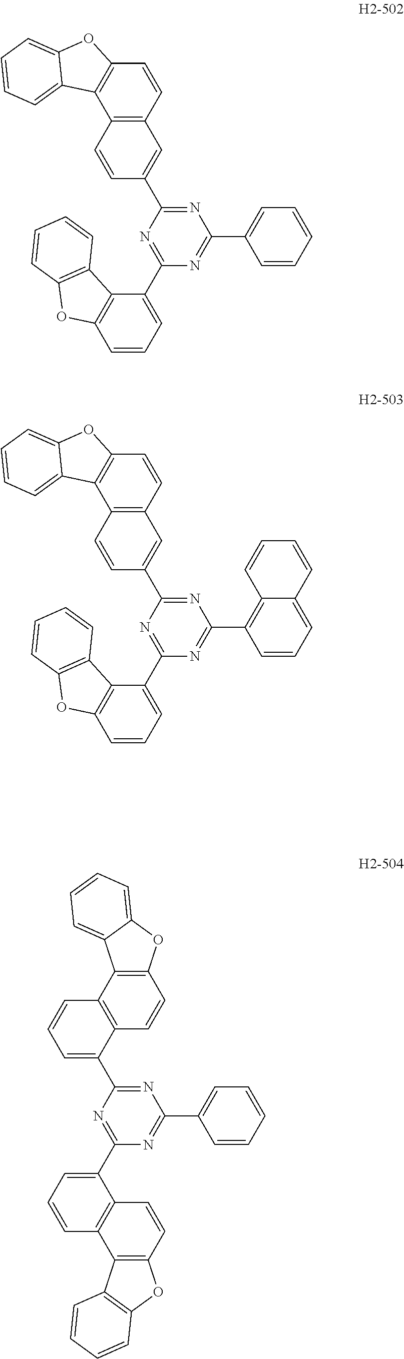 [SMILES: c1ccc(-c2nc(-c3ccc4c(ccc5oc6ccccc6c54)c3)nc(-c3cccc4oc5ccccc5c34)n2)cc1 ; c1ccc2c(-c3nc(-c4ccc5c(ccc6oc7ccccc7c65)c4)nc(-c4cccc5oc6ccccc6c45)n3)cccc2c1 ; c1ccc(-c2nc(-c3cccc4c3ccc3oc5ccccc5c34)nc(-c3cccc4c3ccc3oc5ccccc5c34)n2)cc1]

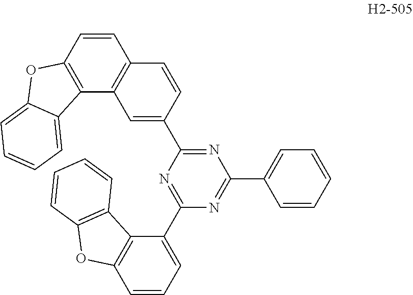 [SMILES: c1ccc(-c2nc(-c3ccc4ccc5oc6ccccc6c5c4c3)nc(-c3cccc4oc5ccccc5c34)n2)cc1]

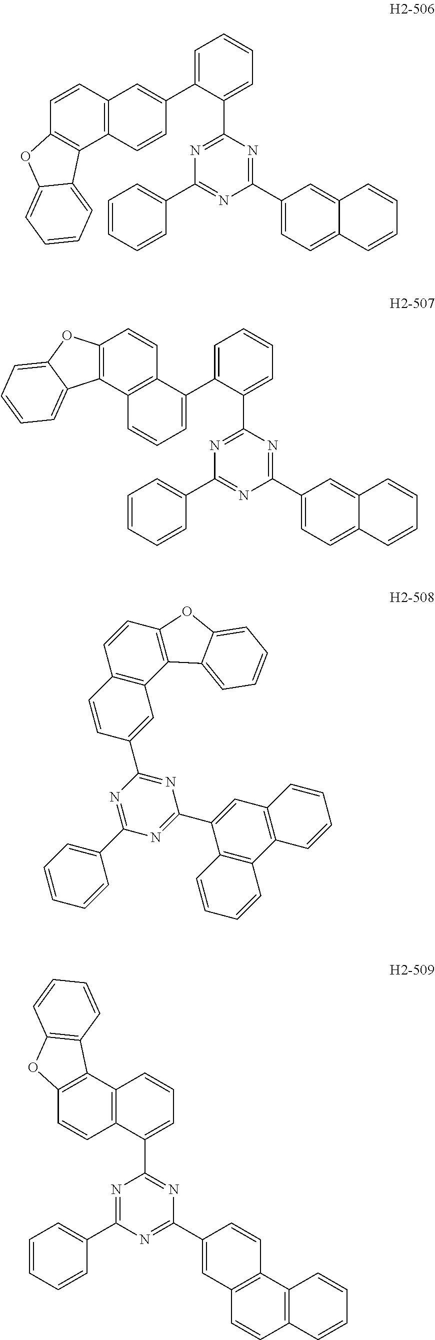 [SMILES: c1ccc(-c2nc(-c3ccc4ccccc4c3)nc(-c3ccccc3-c3ccc4c(ccc5oc6ccccc6c54)c3)n2)cc1 ; c1ccc(-c2nc(-c3ccc4ccccc4c3)nc(-c3ccccc3-c3cccc4c3ccc3oc5ccccc5c34)n2)cc1 ; c1ccc(-c2nc(-c3ccc4ccc5oc6ccccc6c5c4c3)nc(-c3cc4ccccc4c4ccccc34)n2)cc1 ; c1ccc(-c2nc(-c3ccc4c(ccc5ccccc54)c3)nc(-c3cccc4c3ccc3oc5ccccc5c34)n2)cc1]

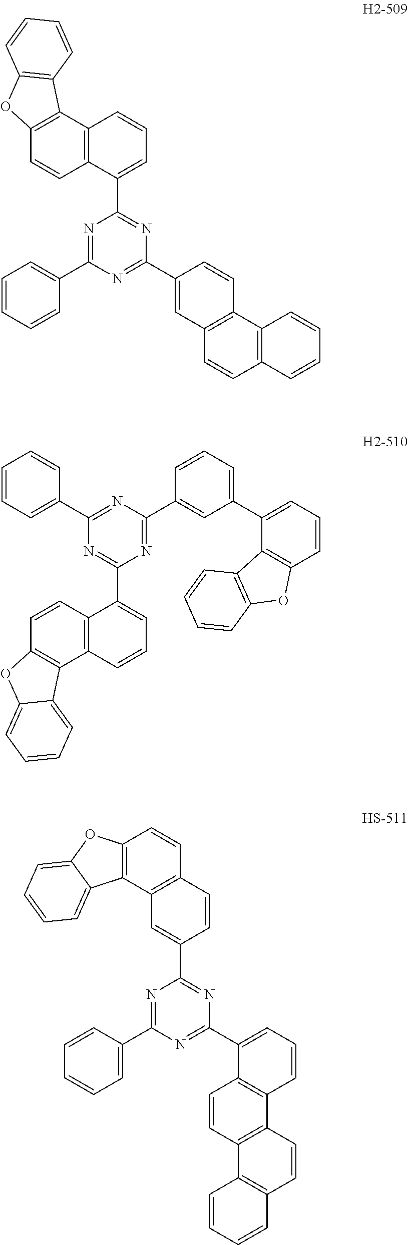 [SMILES: c1ccc(-c2nc(-c3ccc4c(ccc5ccccc54)c3)nc(-c3cccc4c3ccc3oc5ccccc5c34)n2)cc1 ; c1ccc(-c2nc(-c3cccc(-c4cccc5oc6ccccc6c45)c3)nc(-c3cccc4c3ccc3oc5ccccc5c34)n2)cc1 ; c1ccc(-c2nc(-c3ccc4ccc5oc6ccccc6c5c4c3)nc(-c3cccc4c3ccc3c5ccccc5ccc43)n2)cc1]

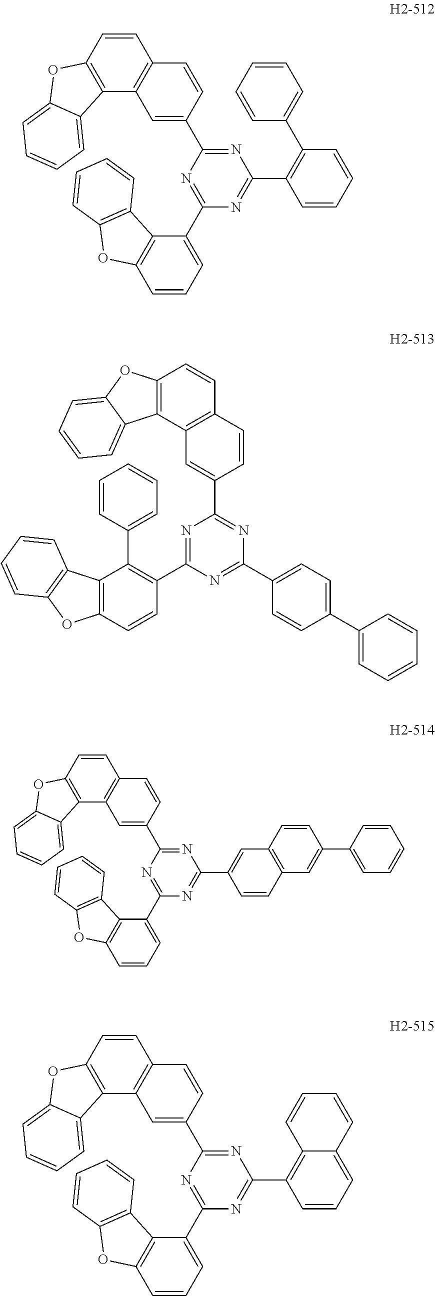 [SMILES: c1ccc(-c2ccccc2-c2nc(-c3ccc4ccc5oc6ccccc6c5c4c3)nc(-c3cccc4oc5ccccc5c34)n2)cc1 ; c1ccc(-c2ccc(-c3nc(-c4ccc5ccc6oc7ccccc7c6c5c4)nc(-c4ccc5oc6ccccc6c5c4-c4ccccc4)n3)cc2)cc1 ; c1ccc(-c2ccc3cc(-c4nc(-c5ccc6ccc7oc8ccccc8c7c6c5)nc(-c5cccc6oc7ccccc7c56)n4)ccc3c2)cc1 ; c1ccc2c(-c3nc(-c4ccc5ccc6oc7ccccc7c6c5c4)nc(-c4cccc5oc6ccccc6c45)n3)cccc2c1]

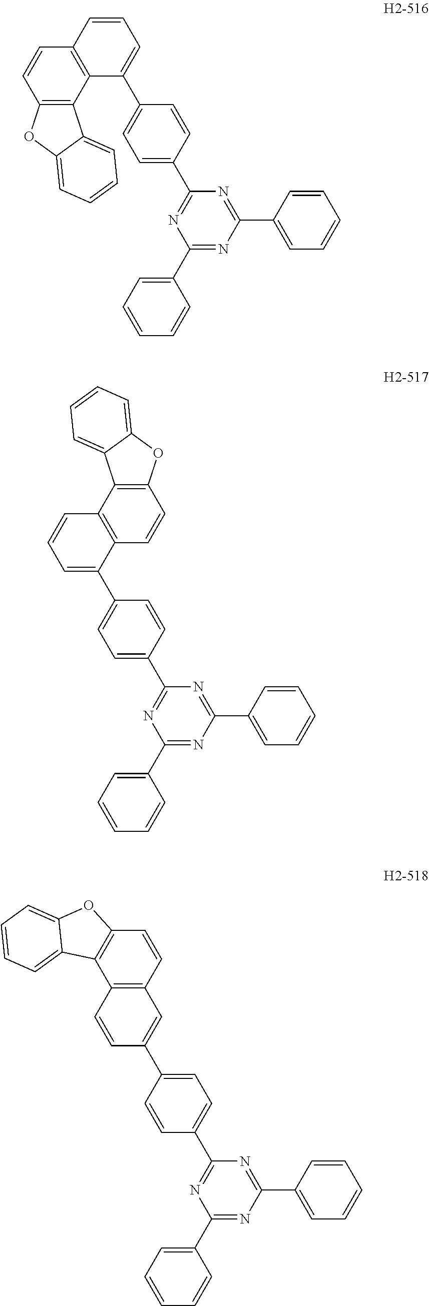 [SMILES: c1ccc(-c2nc(-c3ccccc3)nc(-c3ccc(-c4cccc5ccc6oc7ccccc7c6c45)cc3)n2)cc1 ; c1ccc(-c2nc(-c3ccccc3)nc(-c3ccc(-c4cccc5c4ccc4oc6ccccc6c45)cc3)n2)cc1 ; c1ccc(-c2nc(-c3ccccc3)nc(-c3ccc(-c4ccc5c(ccc6oc7ccccc7c65)c4)cc3)n2)cc1]

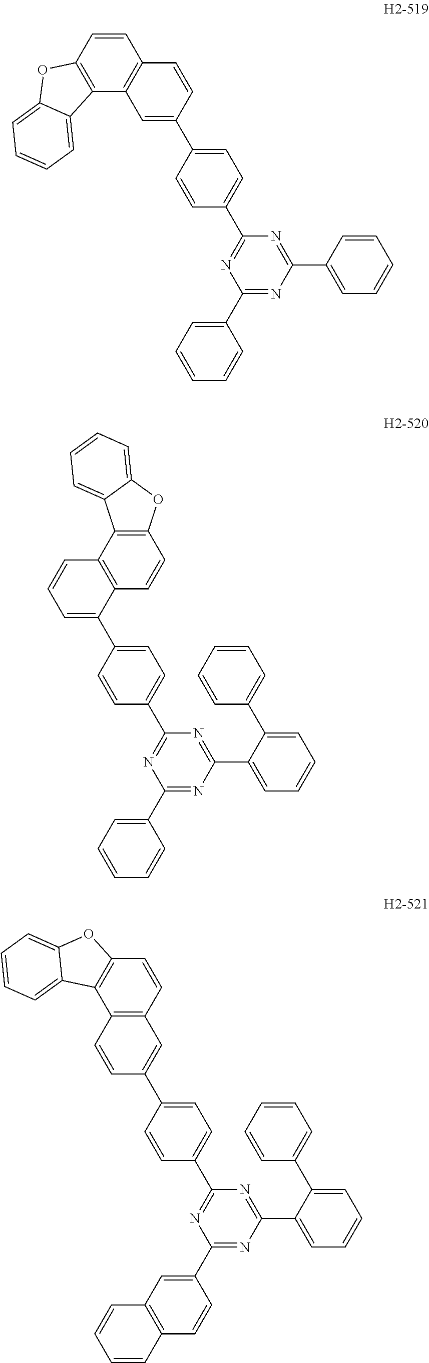 [SMILES: c1ccc(-c2nc(-c3ccccc3)nc(-c3ccc(-c4ccc5ccc6oc7ccccc7c6c5c4)cc3)n2)cc1 ; c1ccc(-c2nc(-c3ccc(-c4cccc5c4ccc4oc6ccccc6c45)cc3)nc(-c3ccccc3-c3ccccc3)n2)cc1 ; c1ccc(-c2ccccc2-c2nc(-c3ccc(-c4ccc5c(ccc6oc7ccccc7c65)c4)cc3)nc(-c3ccc4ccccc4c3)n2)cc1]

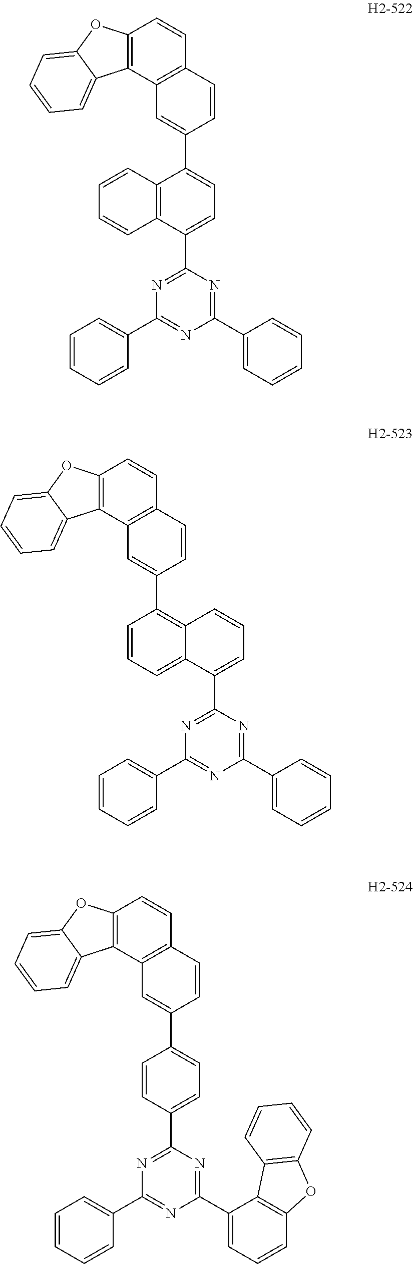 [SMILES: c1ccc(-c2nc(-c3ccccc3)nc(-c3ccc(-c4ccc5ccc6oc7ccccc7c6c5c4)c4ccccc34)n2)cc1 ; c1ccc(-c2nc(-c3ccccc3)nc(-c3cccc4c(-c5ccc6ccc7oc8ccccc8c7c6c5)cccc34)n2)cc1 ; c1ccc(-c2nc(-c3ccc(-c4ccc5ccc6oc7ccccc7c6c5c4)cc3)nc(-c3cccc4oc5ccccc5c34)n2)cc1]

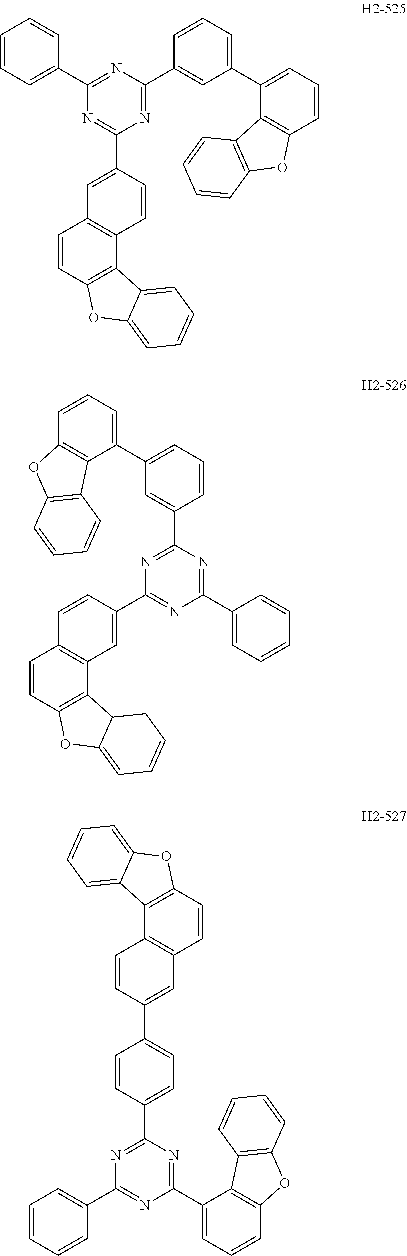 [SMILES: c1ccc(-c2nc(-c3cccc(-c4cccc5oc6ccccc6c45)c3)nc(-c3ccc4c(ccc5oc6ccccc6c54)c3)n2)cc1 ; C1=CCC2C(=C1)Oc1ccc3ccc(-c4nc(-c5ccccc5)nc(-c5cccc(-c6cccc7oc8ccccc8c67)c5)n4)cc3c12 ; c1ccc(-c2nc(-c3ccc(-c4ccc5c(ccc6oc7ccccc7c65)c4)cc3)nc(-c3cccc4oc5ccccc5c34)n2)cc1]

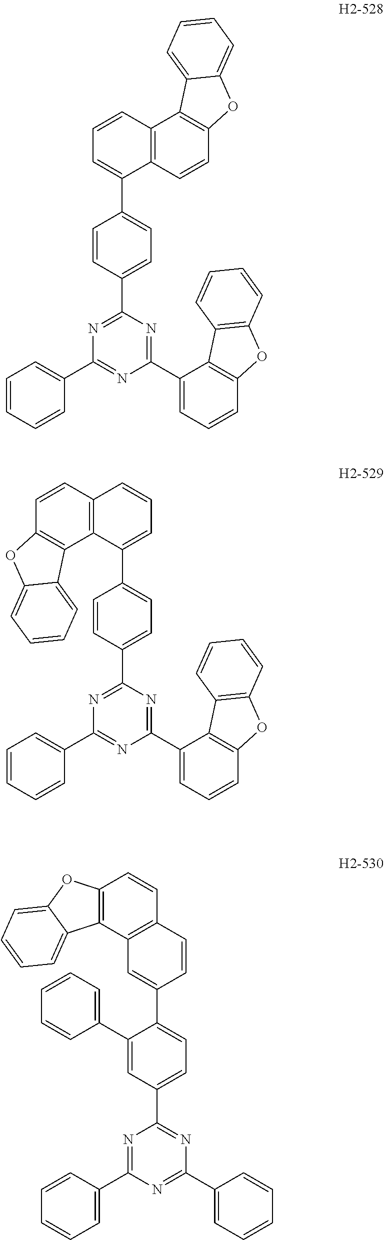 [SMILES: c1ccc(-c2nc(-c3ccc(-c4cccc5c4ccc4oc6ccccc6c45)cc3)nc(-c3cccc4oc5ccccc5c34)n2)cc1 ; c1ccc(-c2nc(-c3ccc(-c4cccc5ccc6oc7ccccc7c6c45)cc3)nc(-c3cccc4oc5ccccc5c34)n2)cc1 ; c1ccc(-c2nc(-c3ccccc3)nc(-c3ccc(-c4ccc5ccc6oc7ccccc7c6c5c4)c(-c4ccccc4)c3)n2)cc1]

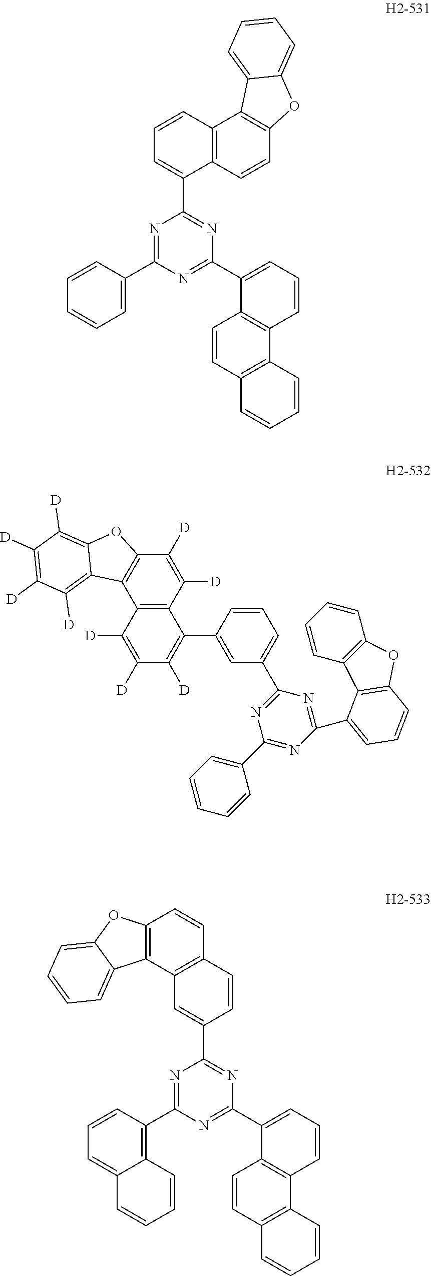 [SMILES: c1ccc(-c2nc(-c3cccc4c3ccc3ccccc34)nc(-c3cccc4c3ccc3oc5ccccc5c34)n2)cc1 ; [2H]c1c([2H])c([2H])c2c(oc3c([2H])c([2H])c4c(-c5cccc(-c6nc(-c7ccccc7)nc(-c7cccc8oc9ccccc9c78)n6)c5)c([2H])c([2H])c([2H])c4c32)c1[2H] ; c1ccc2c(-c3nc(-c4ccc5ccc6oc7ccccc7c6c5c4)nc(-c4cccc5c4ccc4ccccc45)n3)cccc2c1]

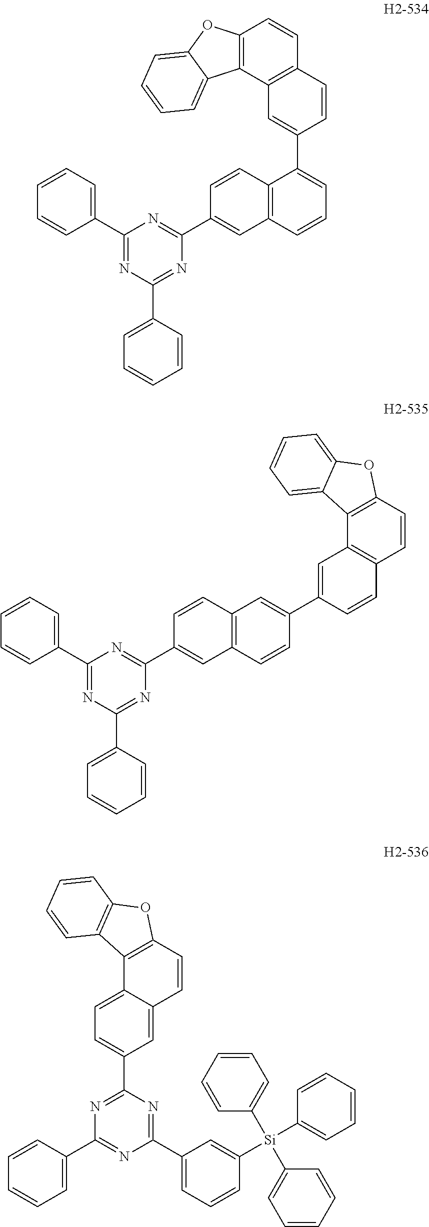 [SMILES: c1ccc(-c2nc(-c3ccccc3)nc(-c3ccc4c(-c5ccc6ccc7oc8ccccc8c7c6c5)cccc4c3)n2)cc1 ; c1ccc(-c2nc(-c3ccccc3)nc(-c3ccc4cc(-c5ccc6ccc7oc8ccccc8c7c6c5)ccc4c3)n2)cc1 ; c1ccc(-c2nc(-c3cccc([Si](c4ccccc4)(c4ccccc4)c4ccccc4)c3)nc(-c3ccc4c(ccc5oc6ccccc6c54)c3)n2)cc1]

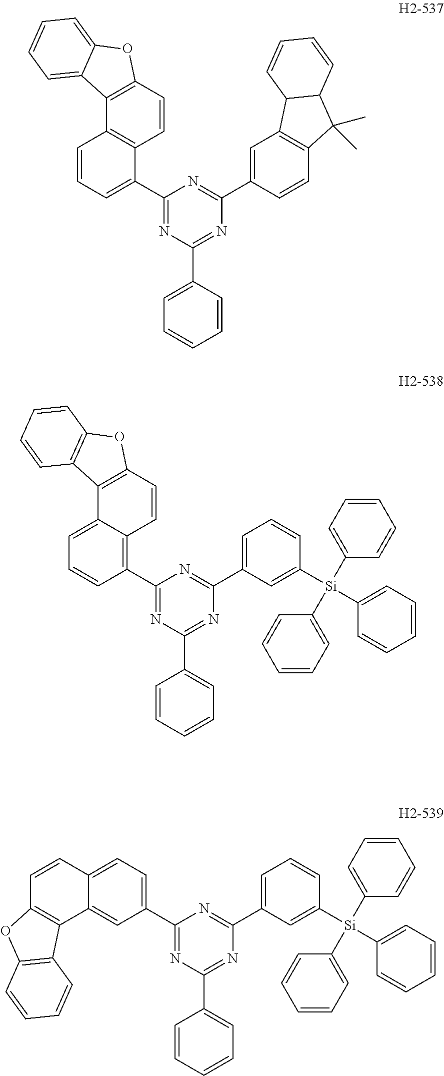 [SMILES: CC1(C)c2ccc(-c3nc(-c4ccccc4)nc(-c4cccc5c4ccc4oc6ccccc6c45)n3)cc2C2C=CC=CC21 ; c1ccc(-c2nc(-c3cccc([Si](c4ccccc4)(c4ccccc4)c4ccccc4)c3)nc(-c3cccc4c3ccc3oc5ccccc5c34)n2)cc1 ; c1ccc(-c2nc(-c3cccc([Si](c4ccccc4)(c4ccccc4)c4ccccc4)c3)nc(-c3ccc4ccc5oc6ccccc6c5c4c3)n2)cc1]

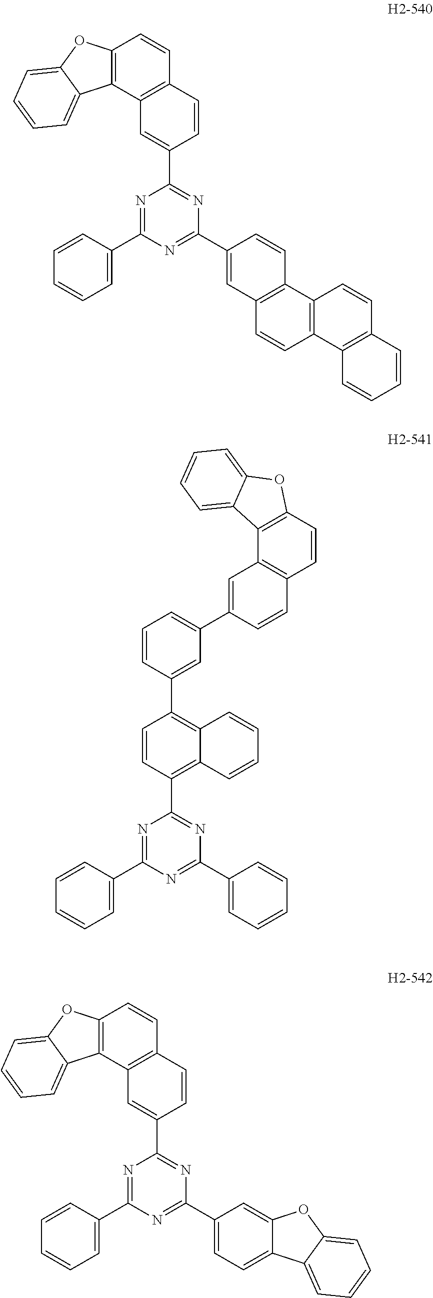 [SMILES: c1ccc(-c2nc(-c3ccc4c(ccc5c6ccccc6ccc45)c3)nc(-c3ccc4ccc5oc6ccccc6c5c4c3)n2)cc1 ; c1ccc(-c2nc(-c3ccccc3)nc(-c3ccc(-c4cccc(-c5ccc6ccc7oc8ccccc8c7c6c5)c4)c4ccccc34)n2)cc1 ; c1ccc(-c2nc(-c3ccc4c(c3)oc3ccccc34)nc(-c3ccc4ccc5oc6ccccc6c5c4c3)n2)cc1]

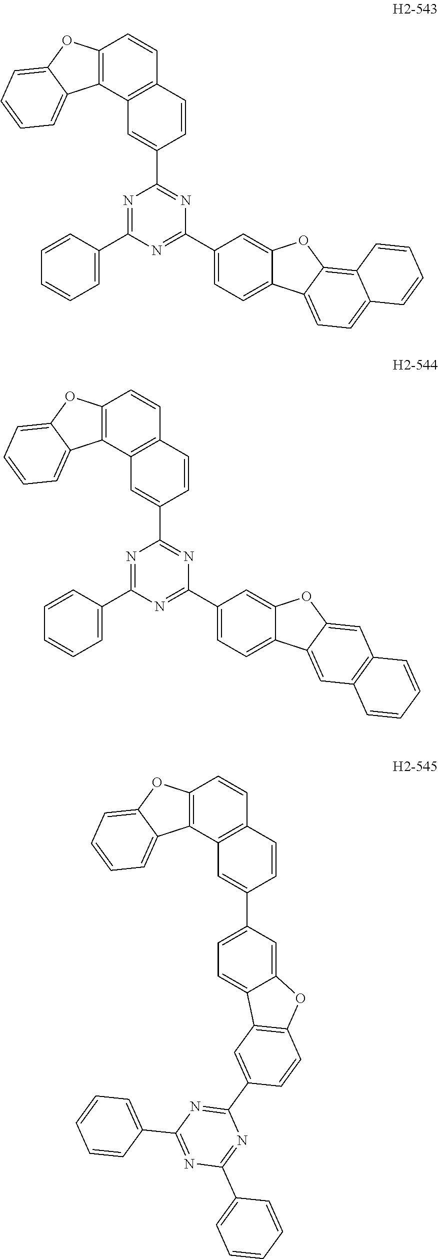 [SMILES: c1ccc(-c2nc(-c3ccc4c(c3)oc3c5ccccc5ccc43)nc(-c3ccc4ccc5oc6ccccc6c5c4c3)n2)cc1 ; c1ccc(-c2nc(-c3ccc4c(c3)oc3cc5ccccc5cc34)nc(-c3ccc4ccc5oc6ccccc6c5c4c3)n2)cc1 ; c1ccc(-c2nc(-c3ccccc3)nc(-c3ccc4oc5cc(-c6ccc7ccc8oc9ccccc9c8c7c6)ccc5c4c3)n2)cc1]

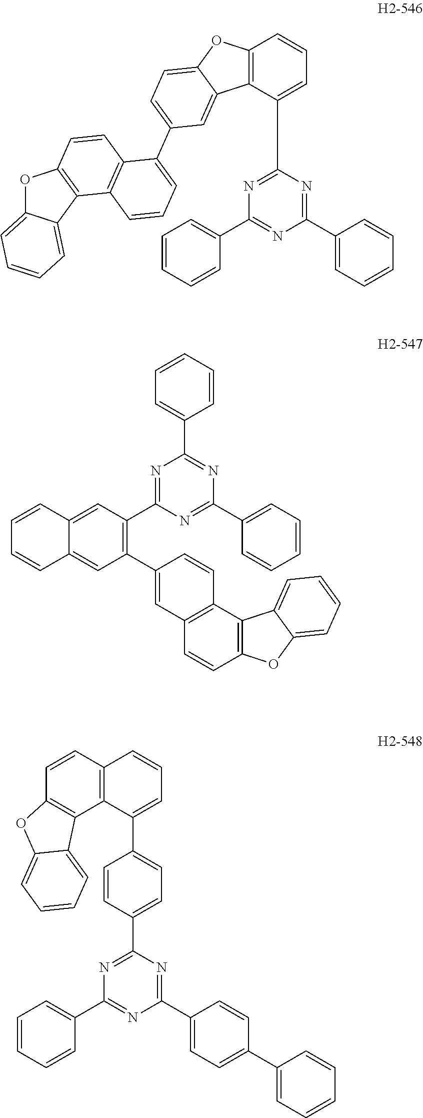 [SMILES: c1ccc(-c2nc(-c3ccccc3)nc(-c3cccc4oc5ccc(-c6cccc7c6ccc6oc8ccccc8c67)cc5c34)n2)cc1 ; c1ccc(-c2nc(-c3ccccc3)nc(-c3cc4ccccc4cc3-c3ccc4c(ccc5oc6ccccc6c54)c3)n2)cc1 ; c1ccc(-c2ccc(-c3nc(-c4ccccc4)nc(-c4ccc(-c5cccc6ccc7oc8ccccc8c7c56)cc4)n3)cc2)cc1]

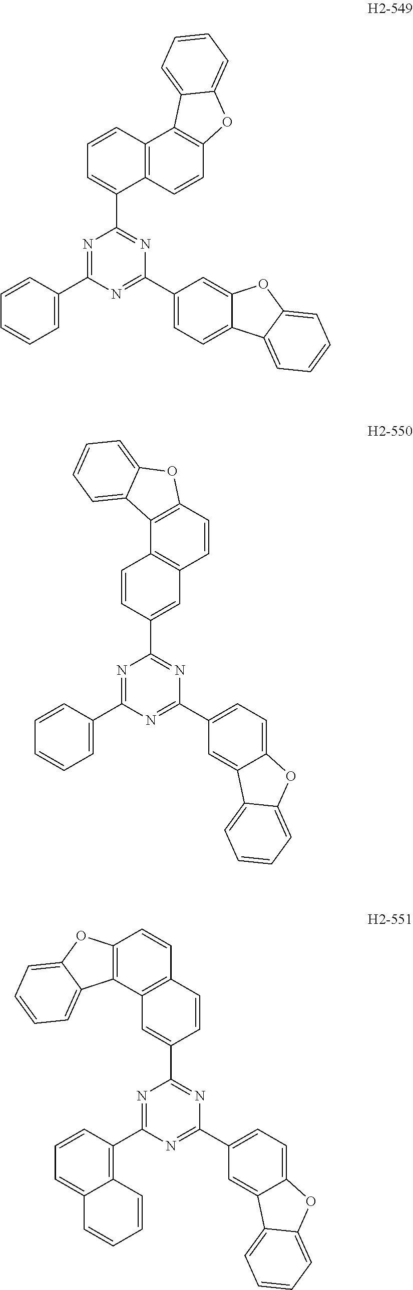 [SMILES: c1ccc(-c2nc(-c3ccc4c(c3)oc3ccccc34)nc(-c3cccc4c3ccc3oc5ccccc5c34)n2)cc1 ; c1ccc(-c2nc(-c3ccc4c(ccc5oc6ccccc6c54)c3)nc(-c3ccc4oc5ccccc5c4c3)n2)cc1 ; c1ccc2c(-c3nc(-c4ccc5oc6ccccc6c5c4)nc(-c4ccc5ccc6oc7ccccc7c6c5c4)n3)cccc2c1]

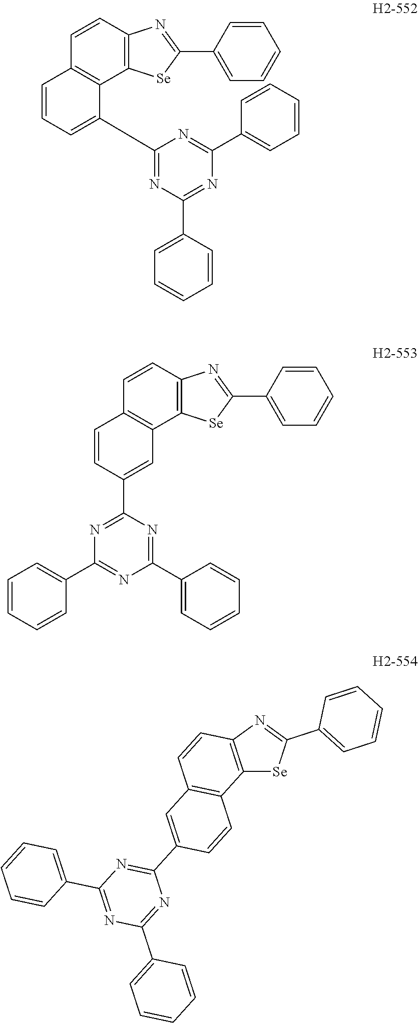 [SMILES: c1ccc(-c2nc(-c3ccccc3)nc(-c3cccc4ccc5nc(-c6ccccc6)[se]c5c34)n2)cc1 ; c1ccc(-c2nc(-c3ccccc3)nc(-c3ccc4ccc5nc(-c6ccccc6)[se]c5c4c3)n2)cc1 ; c1ccc(-c2nc(-c3ccccc3)nc(-c3ccc4c(ccc5nc(-c6ccccc6)[se]c54)c3)n2)cc1]

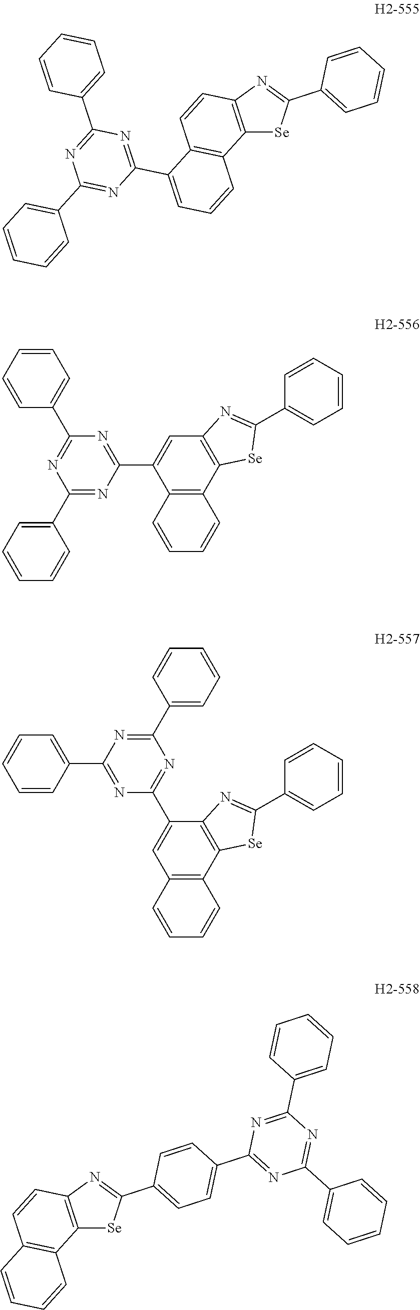 [SMILES: c1ccc(-c2nc(-c3ccccc3)nc(-c3cccc4c3ccc3nc(-c5ccccc5)[se]c34)n2)cc1 ; c1ccc(-c2nc(-c3ccccc3)nc(-c3cc4nc(-c5ccccc5)[se]c4c4ccccc34)n2)cc1 ; c1ccc(-c2nc(-c3ccccc3)nc(-c3cc4ccccc4c4[se]c(-c5ccccc5)nc34)n2)cc1 ; c1ccc(-c2nc(-c3ccccc3)nc(-c3ccc(-c4nc5ccc6ccccc6c5[se]4)cc3)n2)cc1]

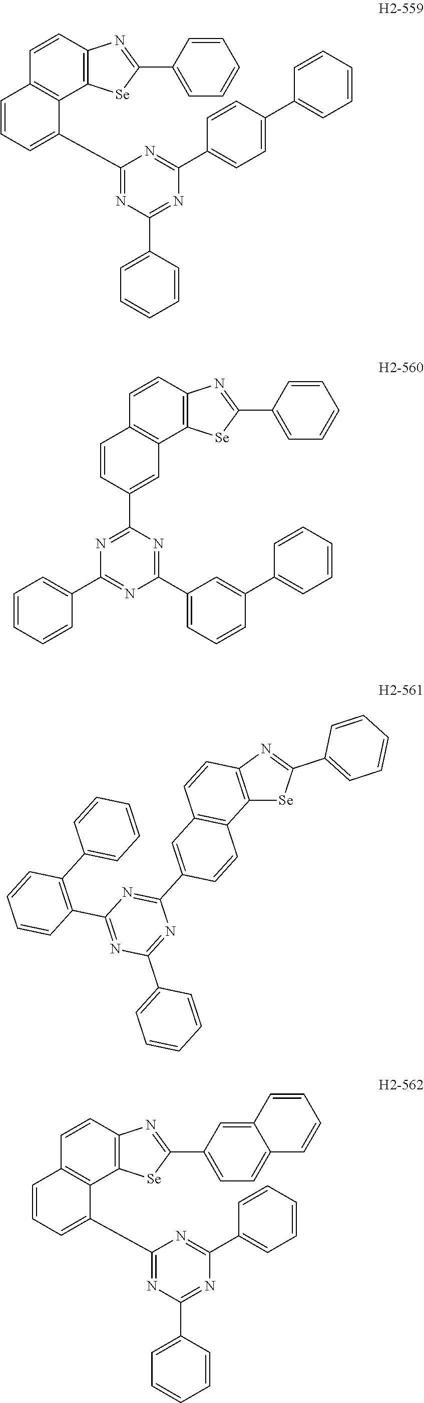 [SMILES: c1ccc(-c2ccc(-c3nc(-c4ccccc4)nc(-c4cccc5ccc6nc(-c7ccccc7)[se]c6c45)n3)cc2)cc1 ; c1ccc(-c2cccc(-c3nc(-c4ccccc4)nc(-c4ccc5ccc6nc(-c7ccccc7)[se]c6c5c4)n3)c2)cc1 ; c1ccc(-c2nc(-c3ccc4c(ccc5nc(-c6ccccc6)[se]c54)c3)nc(-c3ccccc3-c3ccccc3)n2)cc1 ; c1ccc(-c2nc(-c3ccccc3)nc(-c3cccc4ccc5nc(-c6ccc7ccccc7c6)[se]c5c34)n2)cc1]

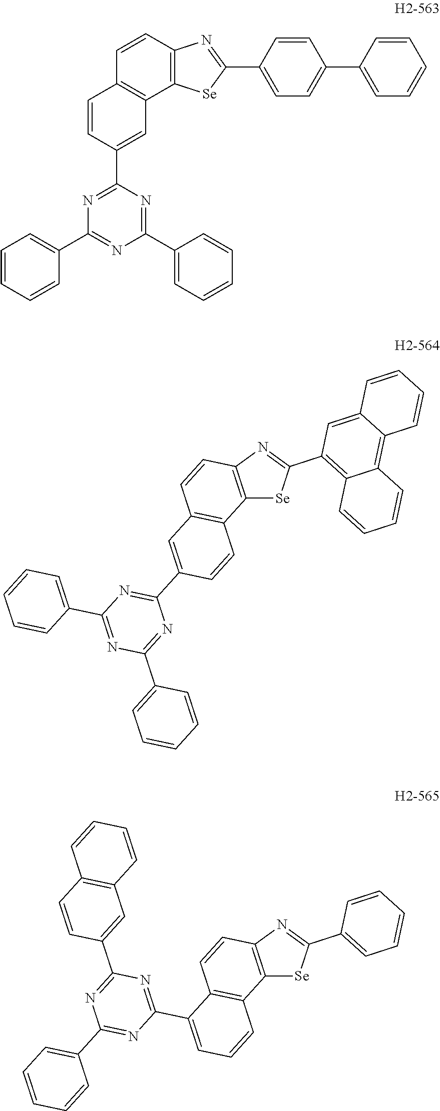 [SMILES: c1ccc(-c2ccc(-c3nc4ccc5ccc(-c6nc(-c7ccccc7)nc(-c7ccccc7)n6)cc5c4[se]3)cc2)cc1 ; c1ccc(-c2nc(-c3ccccc3)nc(-c3ccc4c(ccc5nc(-c6cc7ccccc7c7ccccc67)[se]c54)c3)n2)cc1 ; c1ccc(-c2nc(-c3ccc4ccccc4c3)nc(-c3cccc4c3ccc3nc(-c5ccccc5)[se]c34)n2)cc1]

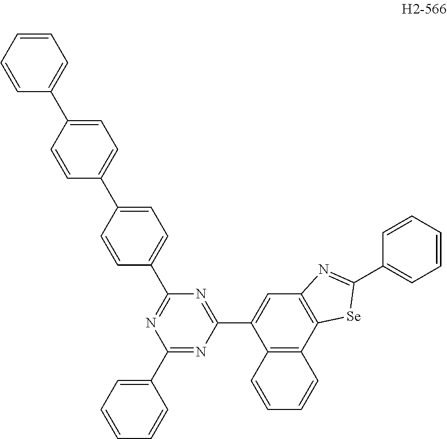 [SMILES: c1ccc(-c2ccc(-c3ccc(-c4nc(-c5ccccc5)nc(-c5cc6nc(-c7ccccc7)[se]c6c6ccccc56)n4)cc3)cc2)cc1]

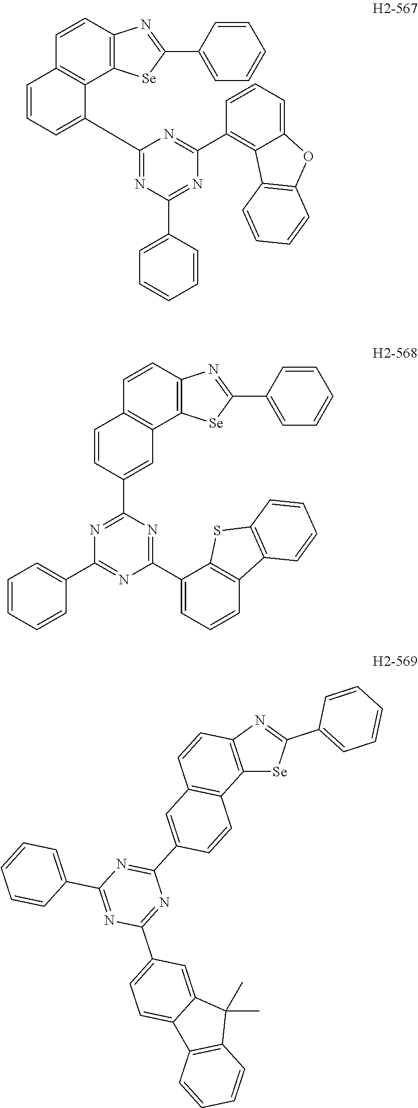 [SMILES: c1ccc(-c2nc(-c3cccc4oc5ccccc5c34)nc(-c3cccc4ccc5nc(-c6ccccc6)[se]c5c34)n2)cc1 ; c1ccc(-c2nc(-c3ccc4ccc5nc(-c6ccccc6)[se]c5c4c3)nc(-c3cccc4c3sc3ccccc34)n2)cc1 ; CC1(C)c2ccccc2-c2ccc(-c3nc(-c4ccccc4)nc(-c4ccc5c(ccc6nc(-c7ccccc7)[se]c65)c4)n3)cc21]

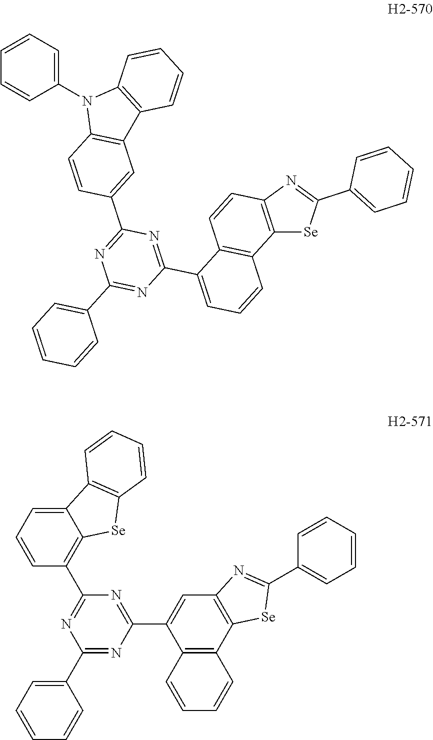 [SMILES: c1ccc(-c2nc(-c3ccc4c(c3)c3ccccc3n4-c3ccccc3)nc(-c3cccc4c3ccc3nc(-c5ccccc5)[se]c34)n2)cc1 ; c1ccc(-c2nc(-c3cc4nc(-c5ccccc5)[se]c4c4ccccc34)nc(-c3cccc4c3[se]c3ccccc34)n2)cc1]

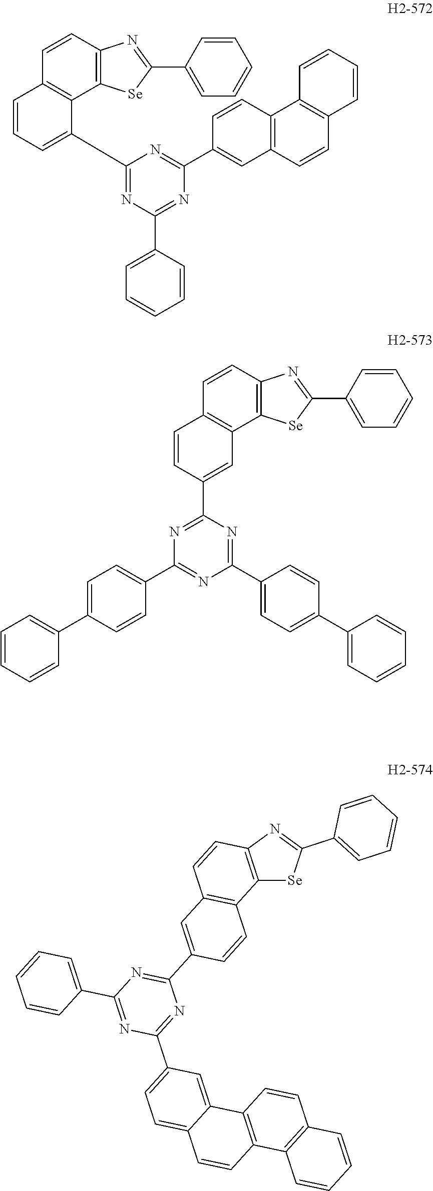 [SMILES: c1ccc(-c2nc(-c3ccc4c(ccc5ccccc54)c3)nc(-c3cccc4ccc5nc(-c6ccccc6)[se]c5c34)n2)cc1 ; c1ccc(-c2ccc(-c3nc(-c4ccc(-c5ccccc5)cc4)nc(-c4ccc5ccc6nc(-c7ccccc7)[se]c6c5c4)n3)cc2)cc1 ; c1ccc(-c2nc(-c3ccc4c(ccc5nc(-c6ccccc6)[se]c54)c3)nc(-c3ccc4ccc5c6ccccc6ccc5c4c3)n2)cc1]

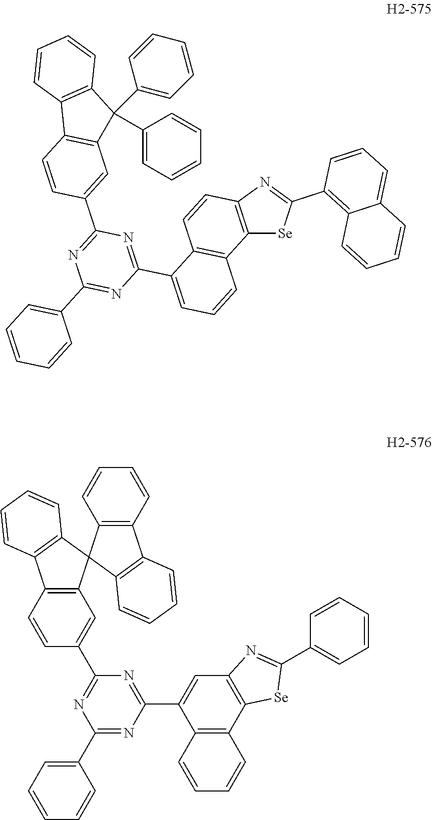 [SMILES: c1ccc(-c2nc(-c3ccc4c(c3)C(c3ccccc3)(c3ccccc3)c3ccccc3-4)nc(-c3cccc4c3ccc3nc(-c5cccc6ccccc56)[se]c34)n2)cc1 ; c1ccc(-c2nc(-c3ccc4c(c3)C3(c5ccccc5-c5ccccc53)c3ccccc3-4)nc(-c3cc4nc(-c5ccccc5)[se]c4c4ccccc34)n2)cc1]

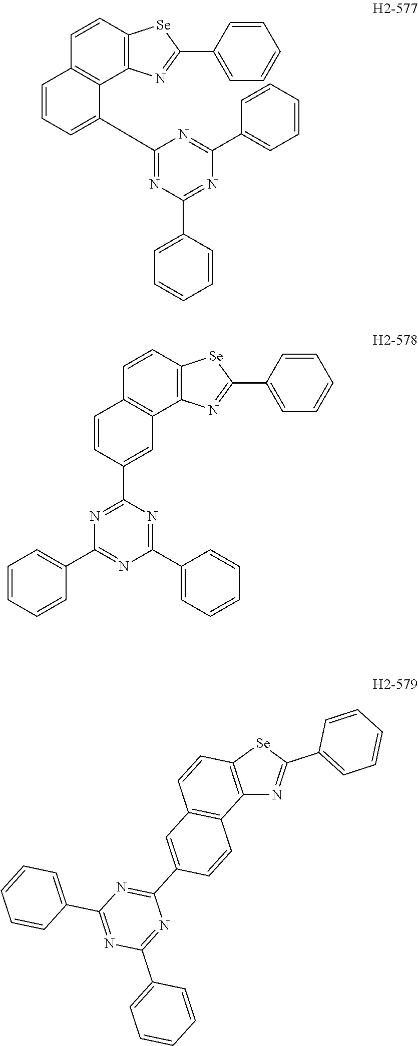 [SMILES: c1ccc(-c2nc(-c3ccccc3)nc(-c3cccc4ccc5[se]c(-c6ccccc6)nc5c34)n2)cc1 ; c1ccc(-c2nc(-c3ccccc3)nc(-c3ccc4ccc5[se]c(-c6ccccc6)nc5c4c3)n2)cc1 ; c1ccc(-c2nc(-c3ccccc3)nc(-c3ccc4c(ccc5[se]c(-c6ccccc6)nc54)c3)n2)cc1]

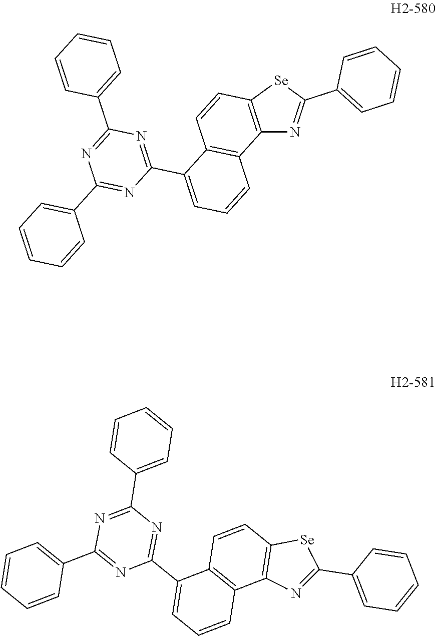 [SMILES: c1ccc(-c2nc(-c3ccccc3)nc(-c3cccc4c3ccc3[se]c(-c5ccccc5)nc34)n2)cc1 ; c1ccc(-c2nc(-c3ccccc3)nc(-c3cccc4c3ccc3[se]c(-c5ccccc5)nc34)n2)cc1]

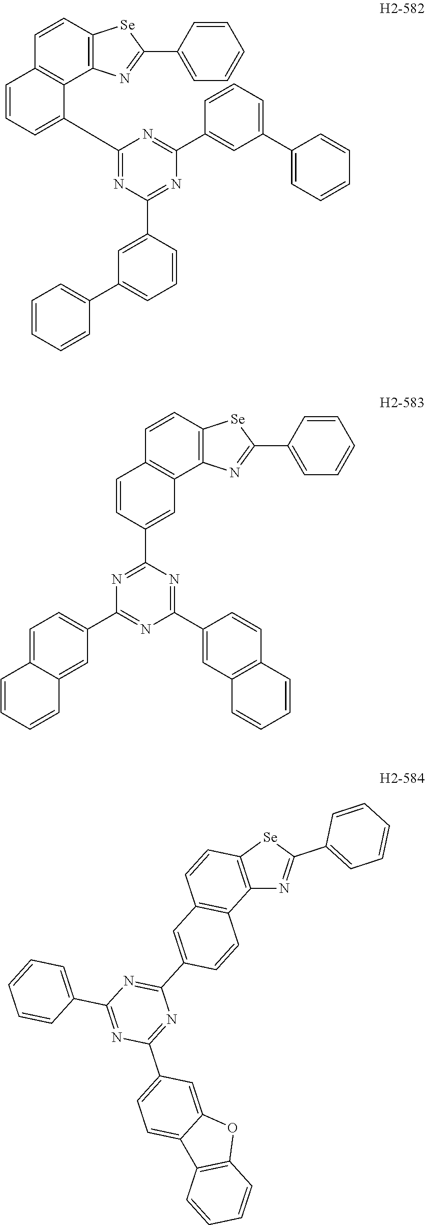 [SMILES: c1ccc(-c2cccc(-c3nc(-c4cccc(-c5ccccc5)c4)nc(-c4cccc5ccc6[se]c(-c7ccccc7)nc6c45)n3)c2)cc1 ; c1ccc(-c2nc3c(ccc4ccc(-c5nc(-c6ccc7ccccc7c6)nc(-c6ccc7ccccc7c6)n5)cc43)[se]2)cc1 ; c1ccc(-c2nc(-c3ccc4c(ccc5[se]c(-c6ccccc6)nc54)c3)nc(-c3ccc4c(c3)oc3ccccc34)n2)cc1]

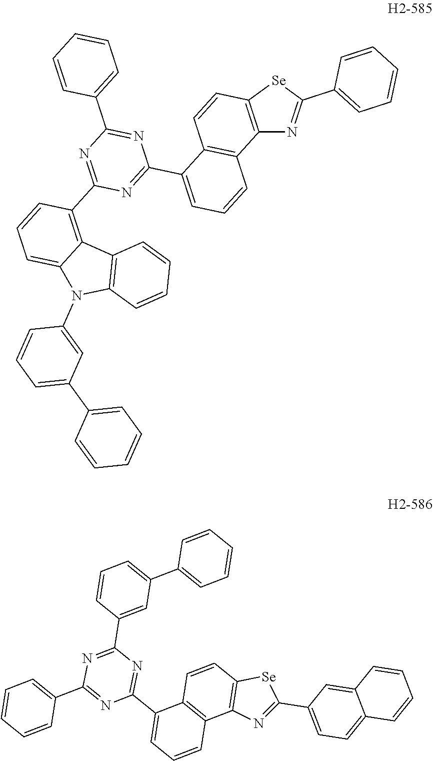 [SMILES: c1ccc(-c2cccc(-n3c4ccccc4c4c(-c5nc(-c6ccccc6)nc(-c6cccc7c6ccc6[se]c(-c8ccccc8)nc67)n5)cccc43)c2)cc1 ; c1ccc(-c2cccc(-c3nc(-c4ccccc4)nc(-c4cccc5c4ccc4[se]c(-c6ccc7ccccc7c6)nc45)n3)c2)cc1]

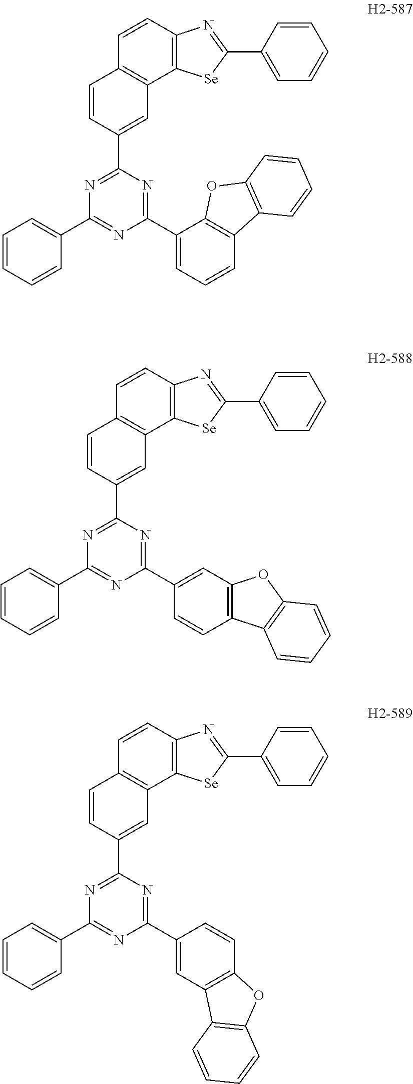 [SMILES: c1ccc(-c2nc(-c3ccc4ccc5nc(-c6ccccc6)[se]c5c4c3)nc(-c3cccc4c3oc3ccccc34)n2)cc1 ; c1ccc(-c2nc(-c3ccc4c(c3)oc3ccccc34)nc(-c3ccc4ccc5nc(-c6ccccc6)[se]c5c4c3)n2)cc1 ; c1ccc(-c2nc(-c3ccc4oc5ccccc5c4c3)nc(-c3ccc4ccc5nc(-c6ccccc6)[se]c5c4c3)n2)cc1]

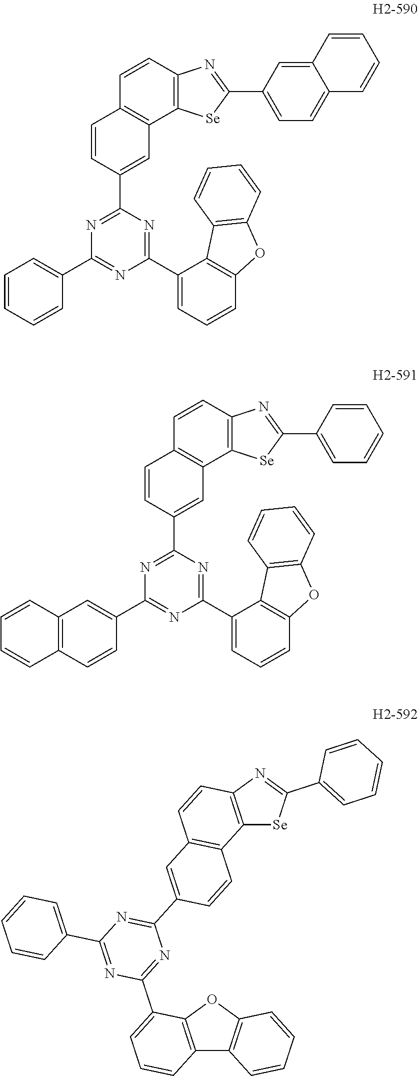 [SMILES: c1ccc(-c2nc(-c3ccc4ccc5nc(-c6ccc7ccccc7c6)[se]c5c4c3)nc(-c3cccc4oc5ccccc5c34)n2)cc1 ; c1ccc(-c2nc3ccc4ccc(-c5nc(-c6ccc7ccccc7c6)nc(-c6cccc7oc8ccccc8c67)n5)cc4c3[se]2)cc1 ; c1ccc(-c2nc(-c3ccc4c(ccc5nc(-c6ccccc6)[se]c54)c3)nc(-c3cccc4c3oc3ccccc34)n2)cc1]

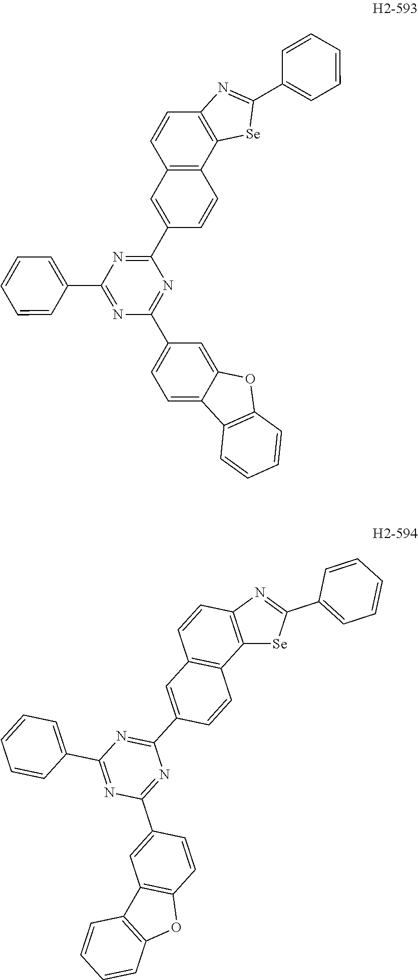 [SMILES: c1ccc(-c2nc(-c3ccc4c(ccc5nc(-c6ccccc6)[se]c54)c3)nc(-c3ccc4c(c3)oc3ccccc34)n2)cc1 ; c1ccc(-c2nc(-c3ccc4c(ccc5nc(-c6ccccc6)[se]c54)c3)nc(-c3ccc4oc5ccccc5c4c3)n2)cc1]

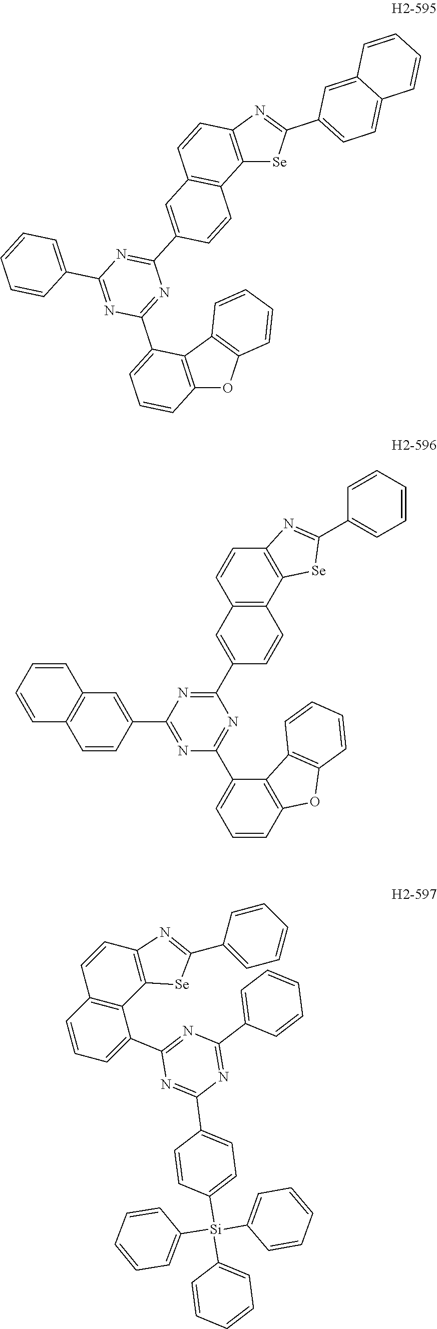 [SMILES: c1ccc(-c2nc(-c3ccc4c(ccc5nc(-c6ccc7ccccc7c6)[se]c54)c3)nc(-c3cccc4oc5ccccc5c34)n2)cc1 ; c1ccc(-c2nc3ccc4cc(-c5nc(-c6ccc7ccccc7c6)nc(-c6cccc7oc8ccccc8c67)n5)ccc4c3[se]2)cc1 ; c1ccc(-c2nc(-c3ccc([Si](c4ccccc4)(c4ccccc4)c4ccccc4)cc3)nc(-c3cccc4ccc5nc(-c6ccccc6)[se]c5c34)n2)cc1]

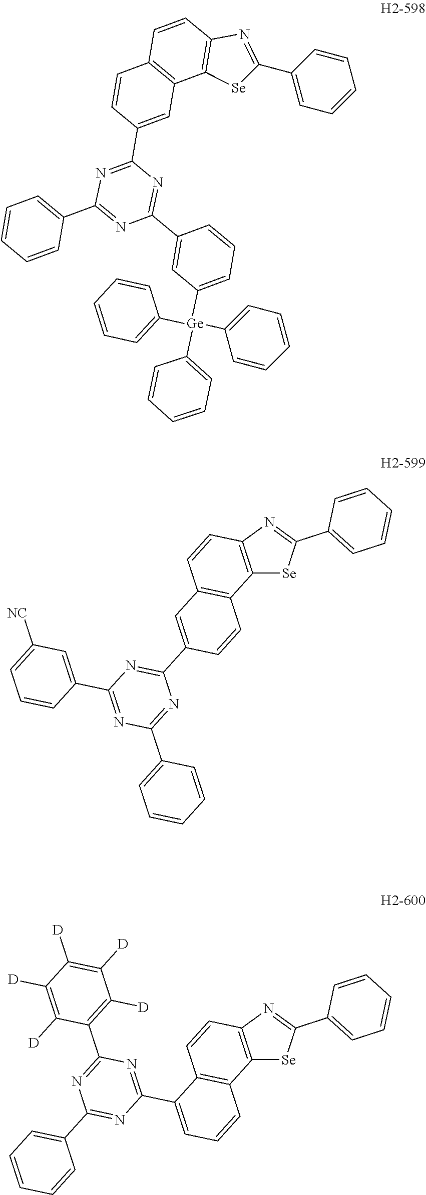

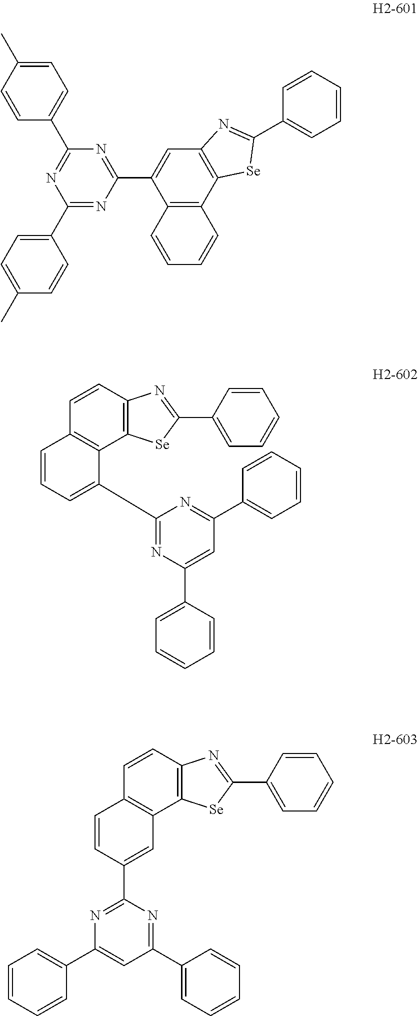 [SMILES: Cc1ccc(-c2nc(-c3ccc(C)cc3)nc(-c3cc4nc(-c5ccccc5)[se]c4c4ccccc34)n2)cc1 ; c1ccc(-c2cc(-c3ccccc3)nc(-c3cccc4ccc5nc(-c6ccccc6)[se]c5c34)n2)cc1 ; c1ccc(-c2cc(-c3ccccc3)nc(-c3ccc4ccc5nc(-c6ccccc6)[se]c5c4c3)n2)cc1]

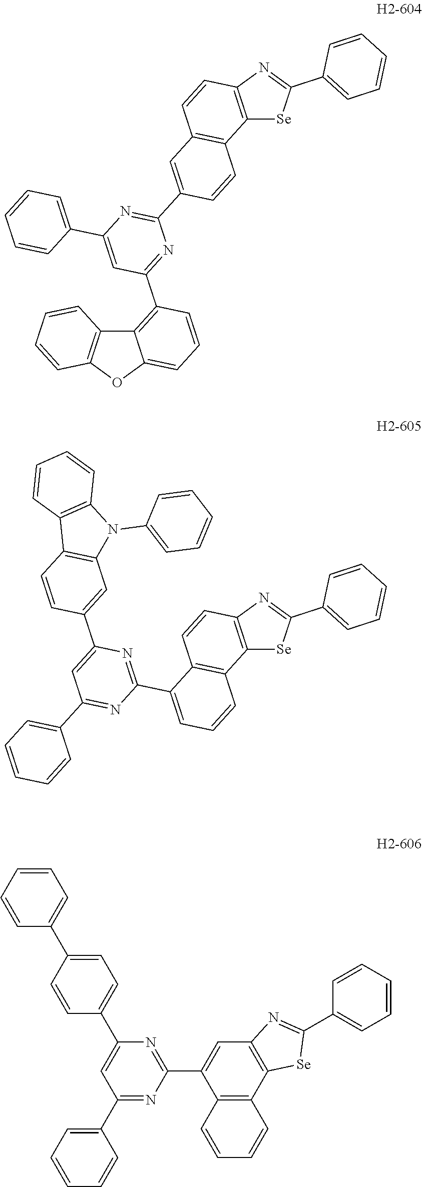 [SMILES: c1ccc(-c2cc(-c3cccc4oc5ccccc5c34)nc(-c3ccc4c(ccc5nc(-c6ccccc6)[se]c54)c3)n2)cc1 ; c1ccc(-c2cc(-c3ccc4c5ccccc5n(-c5ccccc5)c4c3)nc(-c3cccc4c3ccc3nc(-c5ccccc5)[se]c34)n2)cc1 ; c1ccc(-c2ccc(-c3cc(-c4ccccc4)nc(-c4cc5nc(-c6ccccc6)[se]c5c5ccccc45)n3)cc2)cc1]

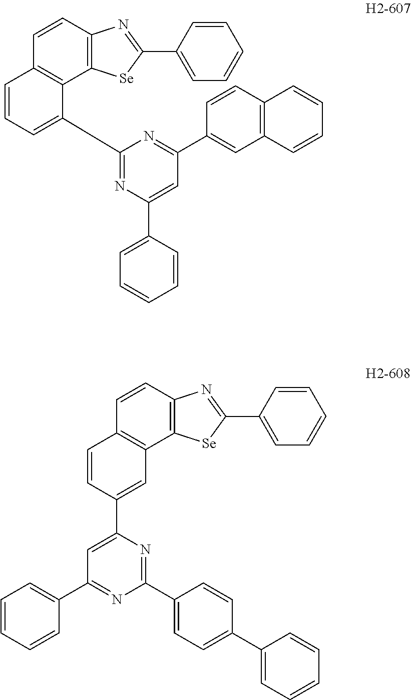 [SMILES: c1ccc(-c2cc(-c3ccc4ccccc4c3)nc(-c3cccc4ccc5nc(-c6ccccc6)[se]c5c34)n2)cc1 ; c1ccc(-c2ccc(-c3nc(-c4ccccc4)cc(-c4ccc5ccc6nc(-c7ccccc7)[se]c6c5c4)n3)cc2)cc1]

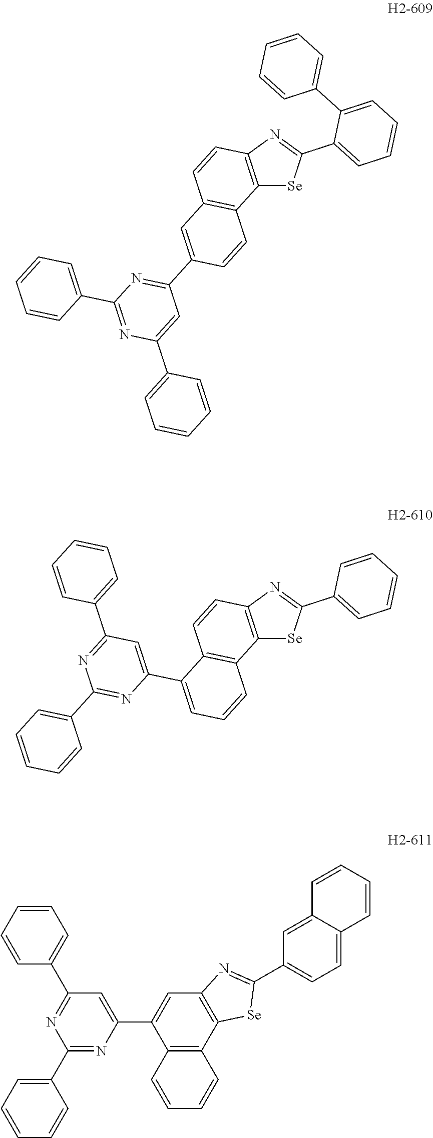 [SMILES: c1ccc(-c2cc(-c3ccc4c(ccc5nc(-c6ccccc6-c6ccccc6)[se]c54)c3)nc(-c3ccccc3)n2)cc1 ; c1ccc(-c2cc(-c3cccc4c3ccc3nc(-c5ccccc5)[se]c34)nc(-c3ccccc3)n2)cc1 ; c1ccc(-c2cc(-c3cc4nc(-c5ccc6ccccc6c5)[se]c4c4ccccc34)nc(-c3ccccc3)n2)cc1]

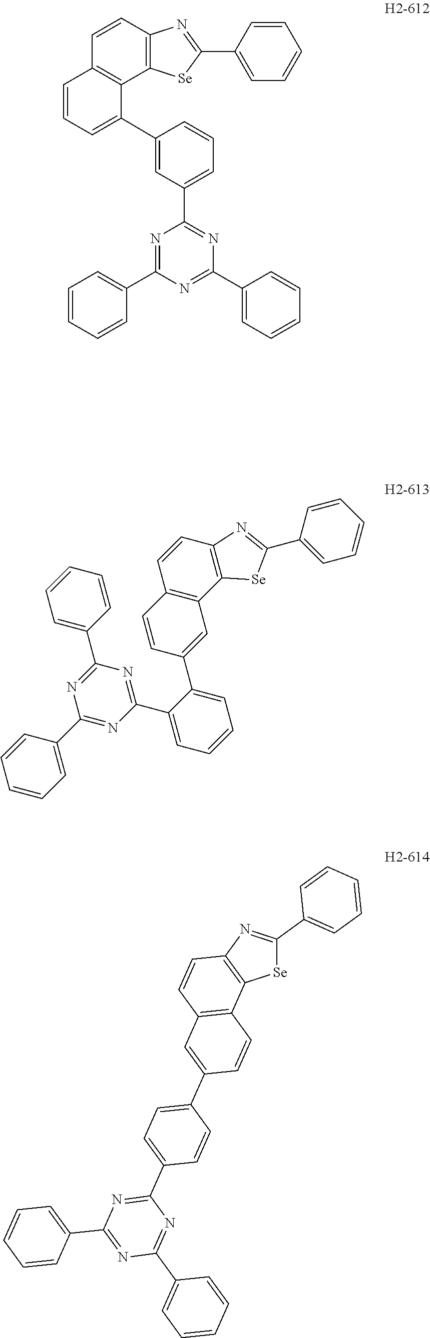 [SMILES: c1ccc(-c2nc(-c3ccccc3)nc(-c3cccc(-c4cccc5ccc6nc(-c7ccccc7)[se]c6c45)c3)n2)cc1 ; c1ccc(-c2nc(-c3ccccc3)nc(-c3ccccc3-c3ccc4ccc5nc(-c6ccccc6)[se]c5c4c3)n2)cc1 ; c1ccc(-c2nc(-c3ccccc3)nc(-c3ccc(-c4ccc5c(ccc6nc(-c7ccccc7)[se]c65)c4)cc3)n2)cc1]

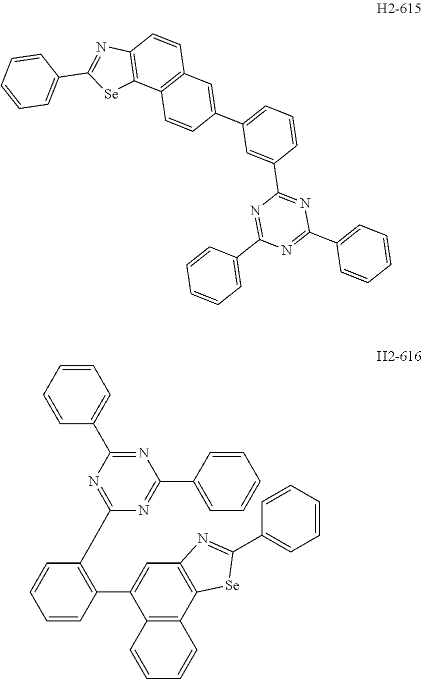 [SMILES: c1ccc(-c2nc(-c3ccccc3)nc(-c3cccc(-c4ccc5c(ccc6nc(-c7ccccc7)[se]c65)c4)c3)n2)cc1 ; c1ccc(-c2nc(-c3ccccc3)nc(-c3ccccc3-c3cc4nc(-c5ccccc5)[se]c4c4ccccc34)n2)cc1]

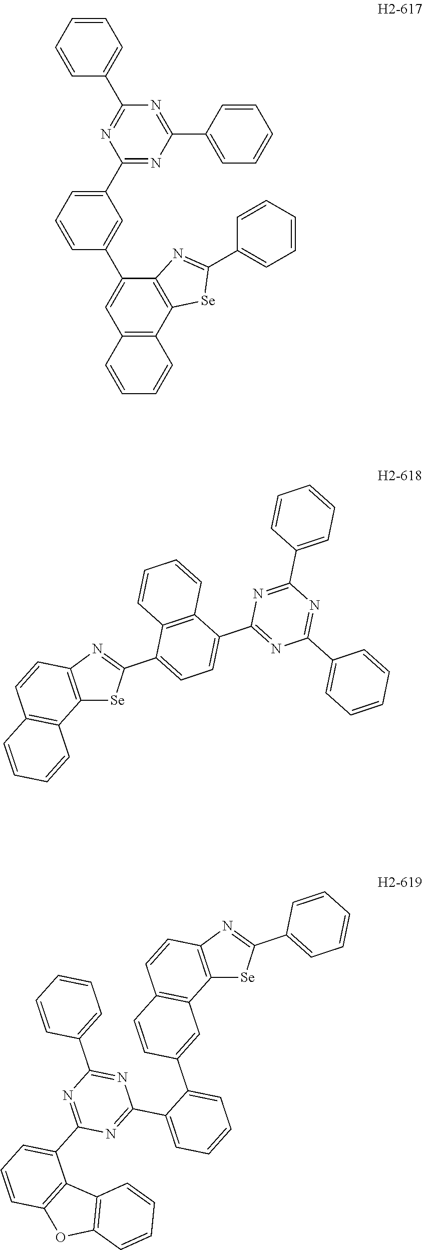 [SMILES: c1ccc(-c2nc(-c3ccccc3)nc(-c3cccc(-c4cc5ccccc5c5[se]c(-c6ccccc6)nc45)c3)n2)cc1 ; c1ccc(-c2nc(-c3ccccc3)nc(-c3ccc(-c4nc5ccc6ccccc6c5[se]4)c4ccccc34)n2)cc1 ; c1ccc(-c2nc(-c3ccccc3-c3ccc4ccc5nc(-c6ccccc6)[se]c5c4c3)nc(-c3cccc4oc5ccccc5c34)n2)cc1]

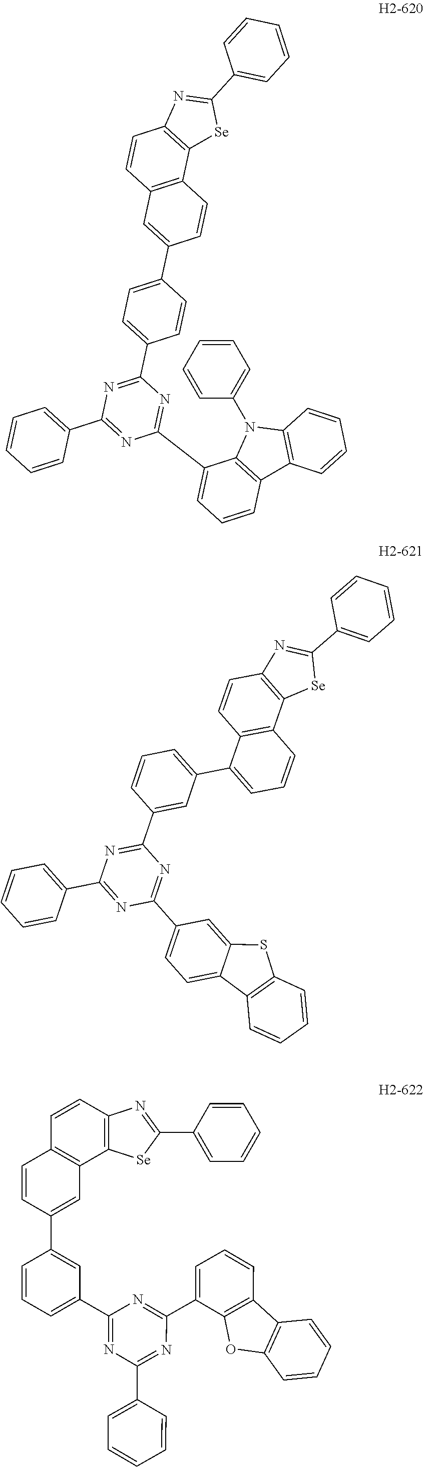 [SMILES: c1ccc(-c2nc(-c3ccc(-c4ccc5c(ccc6nc(-c7ccccc7)[se]c65)c4)cc3)nc(-c3cccc4c5ccccc5n(-c5ccccc5)c34)n2)cc1 ; c1ccc(-c2nc(-c3cccc(-c4cccc5c4ccc4nc(-c6ccccc6)[se]c45)c3)nc(-c3ccc4c(c3)sc3ccccc34)n2)cc1 ; c1ccc(-c2nc(-c3cccc(-c4ccc5ccc6nc(-c7ccccc7)[se]c6c5c4)c3)nc(-c3cccc4c3oc3ccccc34)n2)cc1]

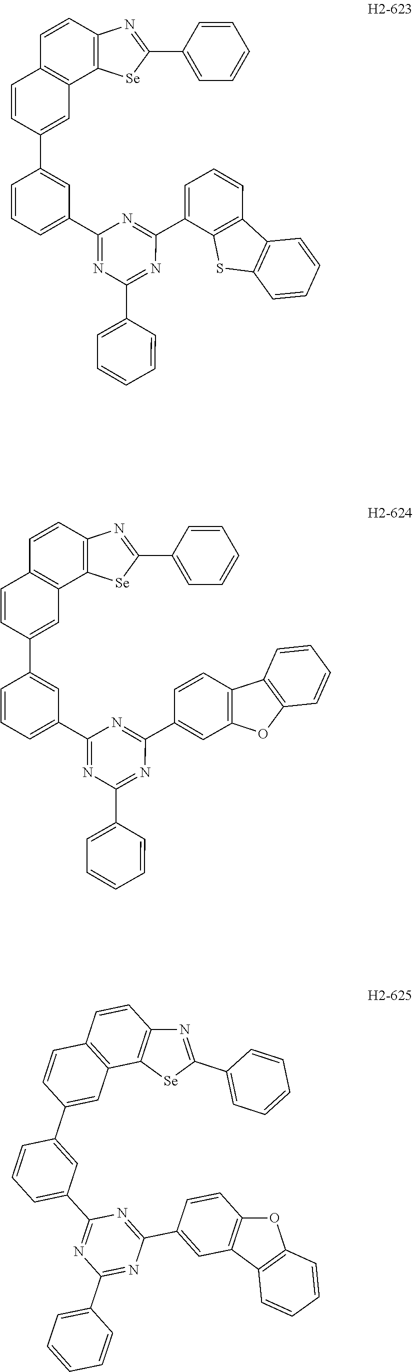 [SMILES: c1ccc(-c2nc(-c3cccc(-c4ccc5ccc6nc(-c7ccccc7)[se]c6c5c4)c3)nc(-c3cccc4c3sc3ccccc34)n2)cc1 ; c1ccc(-c2nc(-c3cccc(-c4ccc5ccc6nc(-c7ccccc7)[se]c6c5c4)c3)nc(-c3ccc4c(c3)oc3ccccc34)n2)cc1 ; c1ccc(-c2nc(-c3cccc(-c4ccc5ccc6nc(-c7ccccc7)[se]c6c5c4)c3)nc(-c3ccc4oc5ccccc5c4c3)n2)cc1]

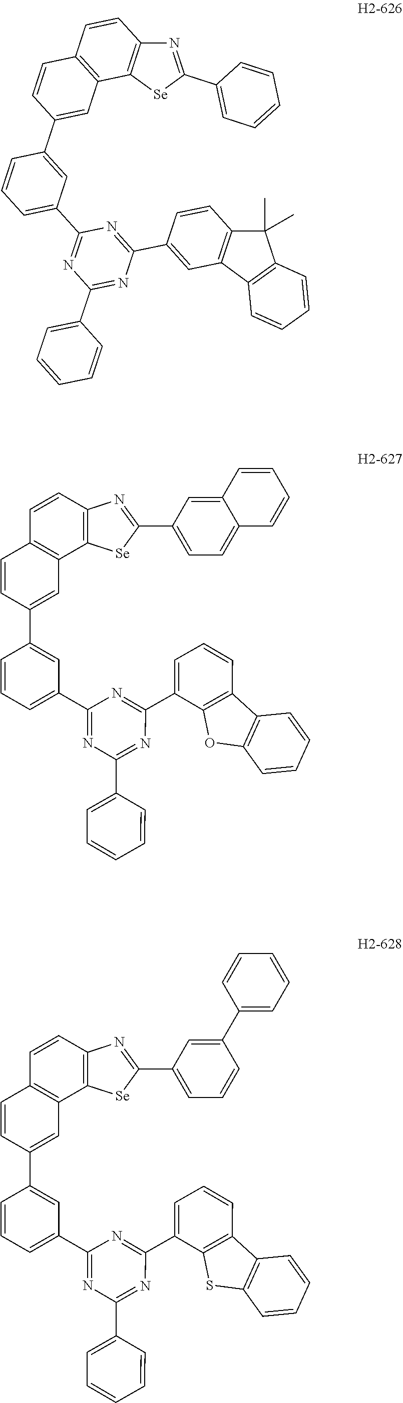 [SMILES: CC1(C)c2ccccc2-c2cc(-c3nc(-c4ccccc4)nc(-c4cccc(-c5ccc6ccc7nc(-c8ccccc8)[se]c7c6c5)c4)n3)ccc21 ; c1ccc(-c2nc(-c3cccc(-c4ccc5ccc6nc(-c7ccc8ccccc8c7)[se]c6c5c4)c3)nc(-c3cccc4c3oc3ccccc34)n2)cc1 ; c1ccc(-c2cccc(-c3nc4ccc5ccc(-c6cccc(-c7nc(-c8ccccc8)nc(-c8cccc9c8sc8ccccc89)n7)c6)cc5c4[se]3)c2)cc1]

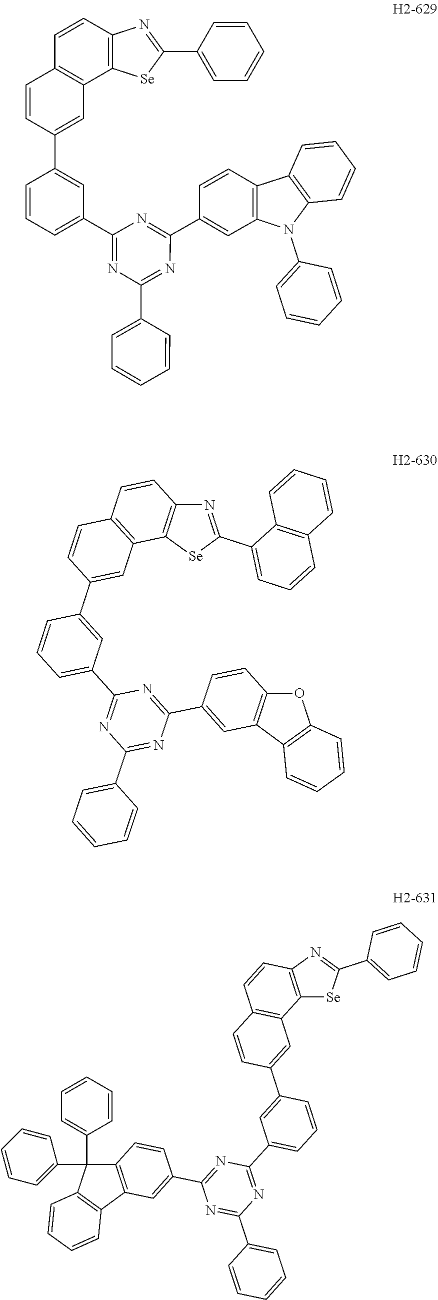 [SMILES: c1ccc(-c2nc(-c3cccc(-c4ccc5ccc6nc(-c7ccccc7)[se]c6c5c4)c3)nc(-c3ccc4c5ccccc5n(-c5ccccc5)c4c3)n2)cc1 ; c1ccc(-c2nc(-c3cccc(-c4ccc5ccc6nc(-c7cccc8ccccc78)[se]c6c5c4)c3)nc(-c3ccc4oc5ccccc5c4c3)n2)cc1 ; c1ccc(-c2nc(-c3cccc(-c4ccc5ccc6nc(-c7ccccc7)[se]c6c5c4)c3)nc(-c3ccc4c(c3)-c3ccccc3C4(c3ccccc3)c3ccccc3)n2)cc1]

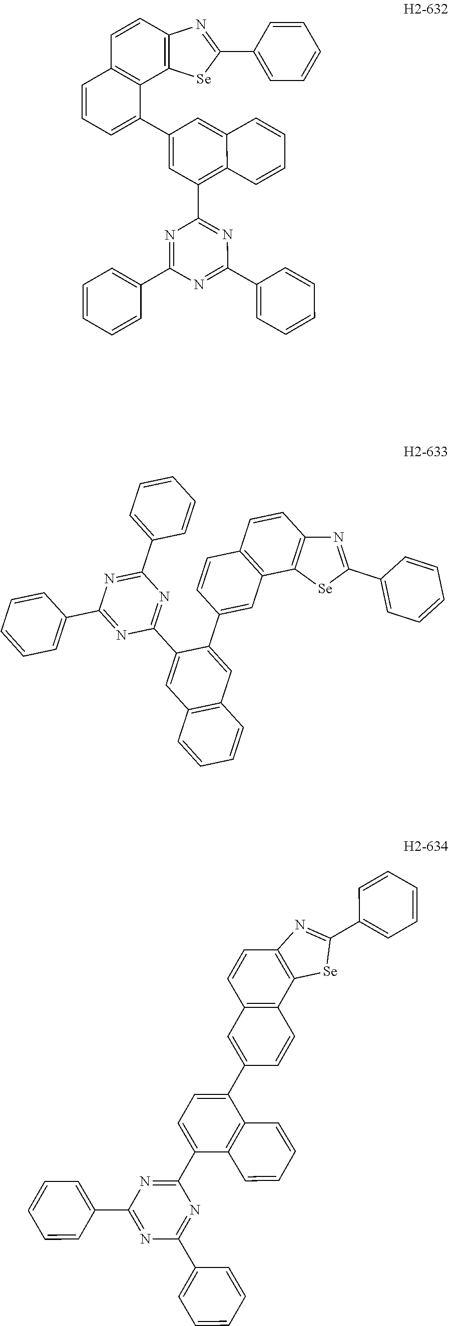 [SMILES: c1ccc(-c2nc(-c3ccccc3)nc(-c3cc(-c4cccc5ccc6nc(-c7ccccc7)[se]c6c45)cc4ccccc34)n2)cc1 ; c1ccc(-c2nc(-c3ccccc3)nc(-c3cc4ccccc4cc3-c3ccc4ccc5nc(-c6ccccc6)[se]c5c4c3)n2)cc1 ; c1ccc(-c2nc(-c3ccccc3)nc(-c3ccc(-c4ccc5c(ccc6nc(-c7ccccc7)[se]c65)c4)c4ccccc34)n2)cc1]

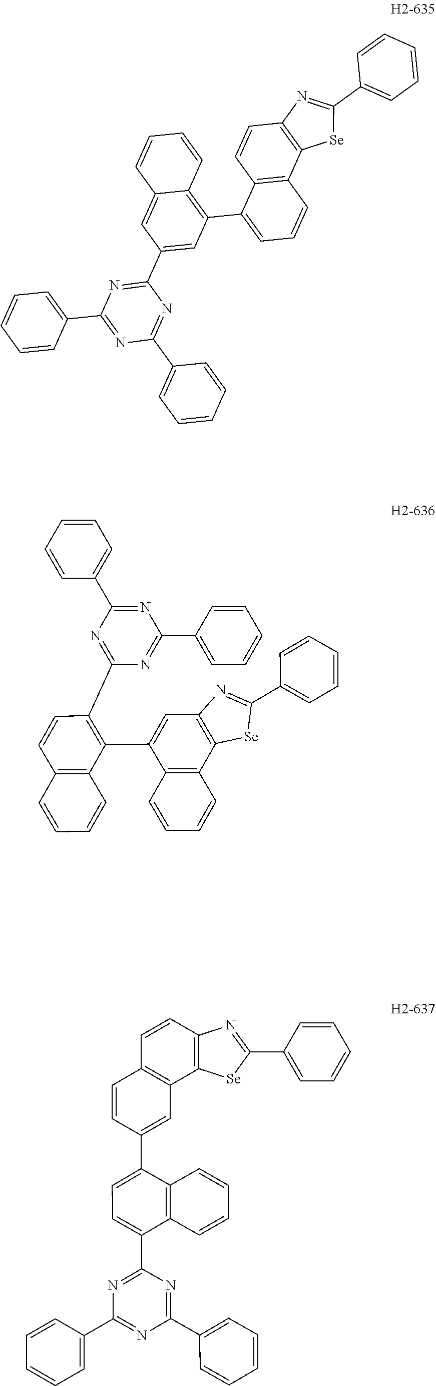 [SMILES: c1ccc(-c2nc(-c3ccccc3)nc(-c3cc(-c4cccc5c4ccc4nc(-c6ccccc6)[se]c45)c4ccccc4c3)n2)cc1 ; c1ccc(-c2nc(-c3ccccc3)nc(-c3ccc4ccccc4c3-c3cc4nc(-c5ccccc5)[se]c4c4ccccc34)n2)cc1 ; c1ccc(-c2nc(-c3ccccc3)nc(-c3ccc(-c4ccc5ccc6nc(-c7ccccc7)[se]c6c5c4)c4ccccc34)n2)cc1]

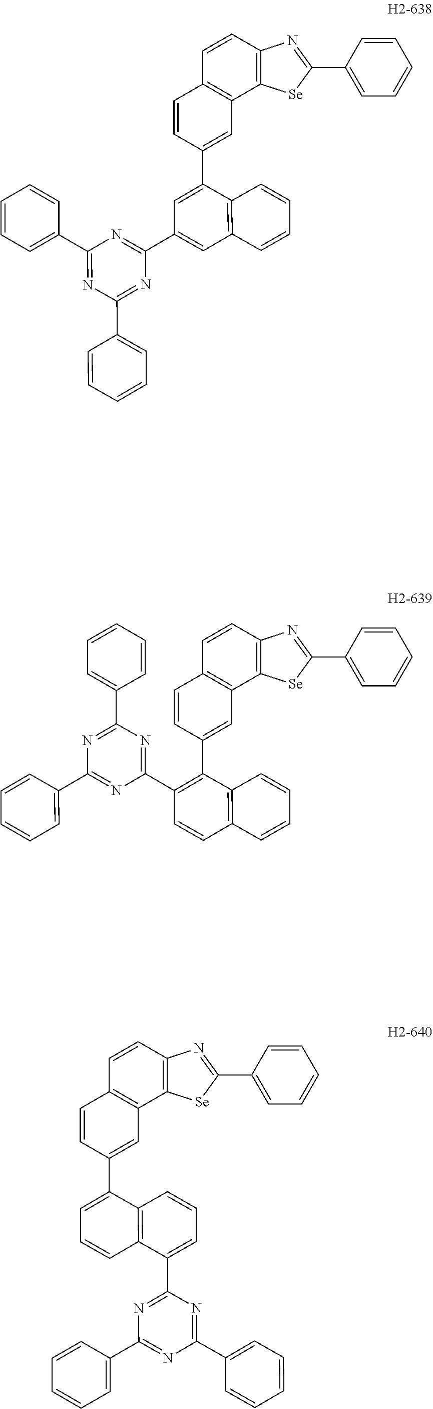 [SMILES: c1ccc(-c2nc(-c3ccccc3)nc(-c3cc(-c4ccc5ccc6nc(-c7ccccc7)[se]c6c5c4)c4ccccc4c3)n2)cc1 ; c1ccc(-c2nc(-c3ccccc3)nc(-c3ccc4ccccc4c3-c3ccc4ccc5nc(-c6ccccc6)[se]c5c4c3)n2)cc1 ; c1ccc(-c2nc(-c3ccccc3)nc(-c3cccc4c(-c5ccc6ccc7nc(-c8ccccc8)[se]c7c6c5)cccc34)n2)cc1]

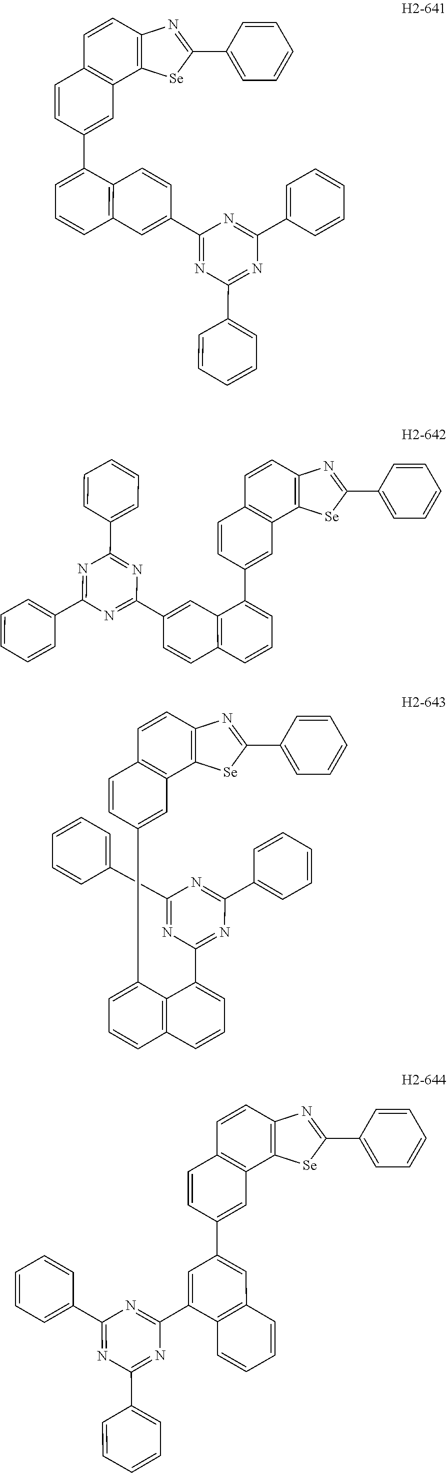 [SMILES: c1ccc(-c2nc(-c3ccccc3)nc(-c3ccc4c(-c5ccc6ccc7nc(-c8ccccc8)[se]c7c6c5)cccc4c3)n2)cc1 ; c1ccc(-c2nc(-c3ccccc3)nc(-c3ccc4cccc(-c5ccc6ccc7nc(-c8ccccc8)[se]c7c6c5)c4c3)n2)cc1 ; c1ccc(-c2nc(-c3ccccc3)nc(-c3cccc4cccc(-c5ccc6ccc7nc(-c8ccccc8)[se]c7c6c5)c34)n2)cc1 ; c1ccc(-c2nc(-c3ccccc3)nc(-c3cc(-c4ccc5ccc6nc(-c7ccccc7)[se]c6c5c4)cc4ccccc34)n2)cc1]

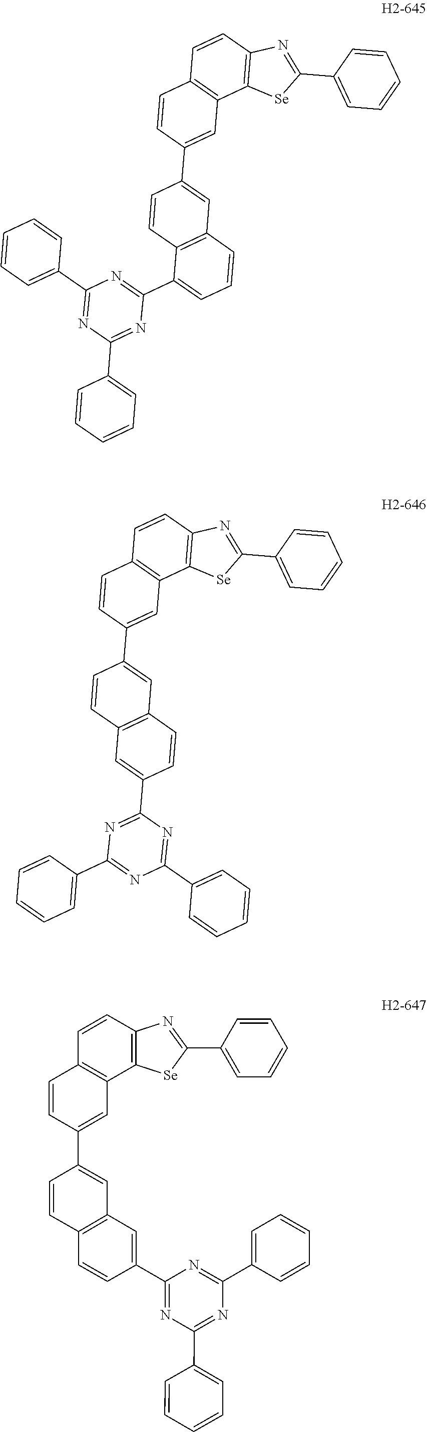 [SMILES: c1ccc(-c2nc(-c3ccccc3)nc(-c3cccc4cc(-c5ccc6ccc7nc(-c8ccccc8)[se]c7c6c5)ccc34)n2)cc1 ; c1ccc(-c2nc(-c3ccccc3)nc(-c3ccc4cc(-c5ccc6ccc7nc(-c8ccccc8)[se]c7c6c5)ccc4c3)n2)cc1 ; c1ccc(-c2nc(-c3ccccc3)nc(-c3ccc4ccc(-c5ccc6ccc7nc(-c8ccccc8)[se]c7c6c5)cc4c3)n2)cc1]

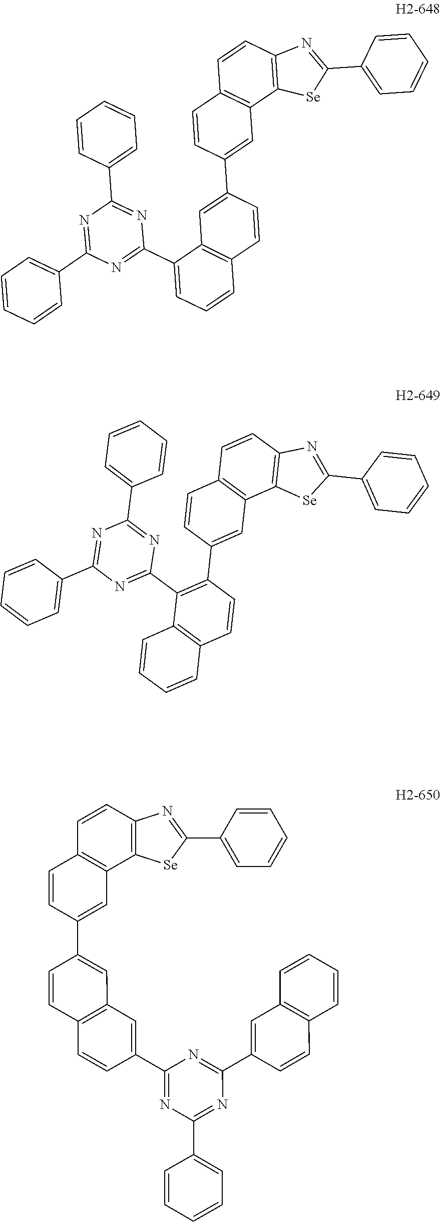 [SMILES: c1ccc(-c2nc(-c3ccccc3)nc(-c3cccc4ccc(-c5ccc6ccc7nc(-c8ccccc8)[se]c7c6c5)cc34)n2)cc1 ; c1ccc(-c2nc(-c3ccccc3)nc(-c3c(-c4ccc5ccc6nc(-c7ccccc7)[se]c6c5c4)ccc4ccccc34)n2)cc1 ; c1ccc(-c2nc(-c3ccc4ccccc4c3)nc(-c3ccc4ccc(-c5ccc6ccc7nc(-c8ccccc8)[se]c7c6c5)cc4c3)n2)cc1]

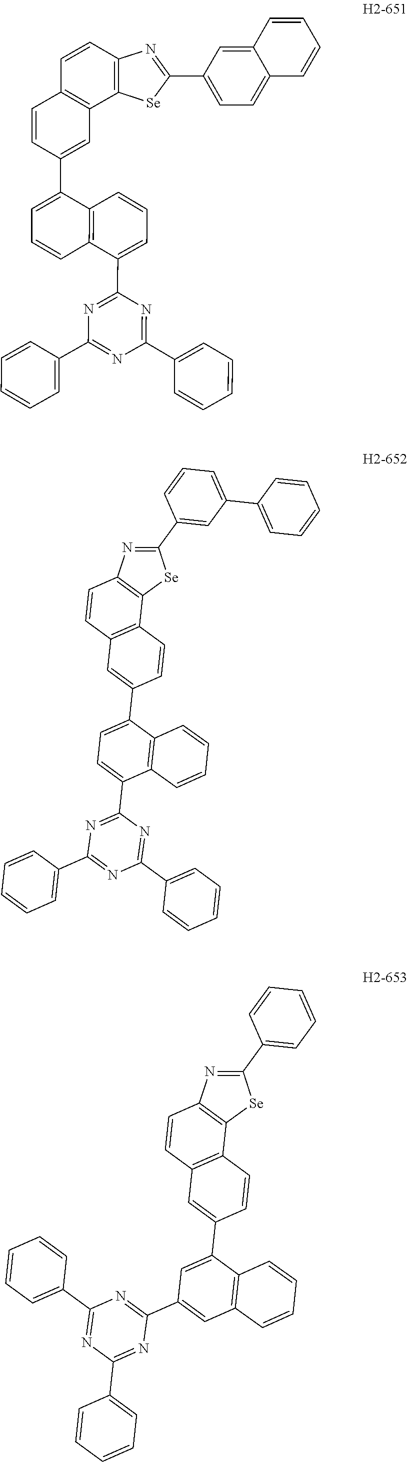 [SMILES: c1ccc(-c2nc(-c3ccccc3)nc(-c3cccc4c(-c5ccc6ccc7nc(-c8ccc9ccccc9c8)[se]c7c6c5)cccc34)n2)cc1 ; c1ccc(-c2cccc(-c3nc4ccc5cc(-c6ccc(-c7nc(-c8ccccc8)nc(-c8ccccc8)n7)c7ccccc67)ccc5c4[se]3)c2)cc1 ; c1ccc(-c2nc(-c3ccccc3)nc(-c3cc(-c4ccc5c(ccc6nc(-c7ccccc7)[se]c65)c4)c4ccccc4c3)n2)cc1]

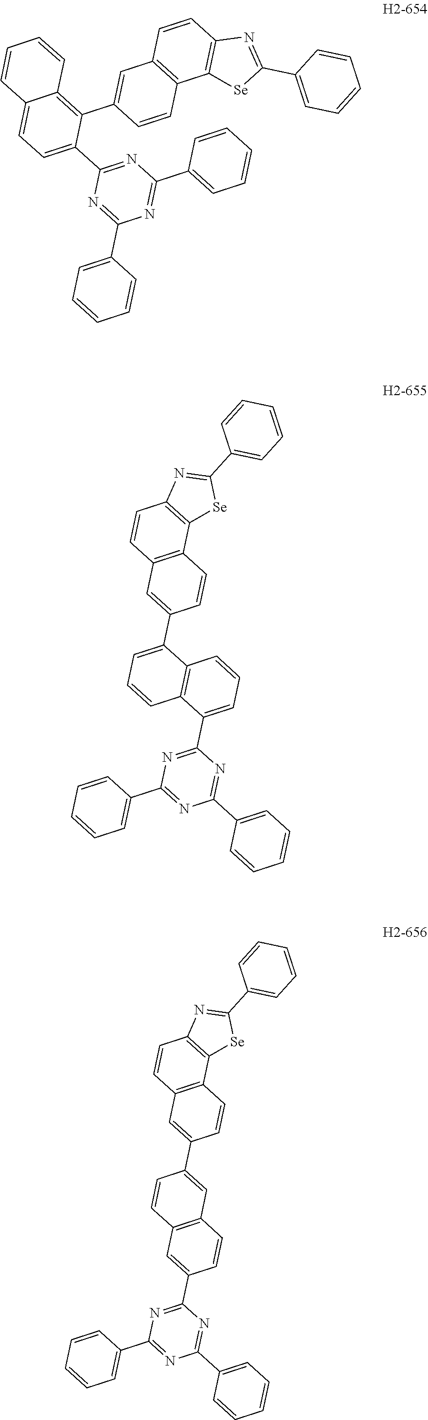 [SMILES: c1ccc(-c2nc(-c3ccccc3)nc(-c3ccc4ccccc4c3-c3ccc4c(ccc5nc(-c6ccccc6)[se]c54)c3)n2)cc1 ; c1ccc(-c2nc(-c3ccccc3)nc(-c3cccc4c(-c5ccc6c(ccc7nc(-c8ccccc8)[se]c76)c5)cccc34)n2)cc1 ; c1ccc(-c2nc(-c3ccccc3)nc(-c3ccc4cc(-c5ccc6c(ccc7nc(-c8ccccc8)[se]c76)c5)ccc4c3)n2)cc1]

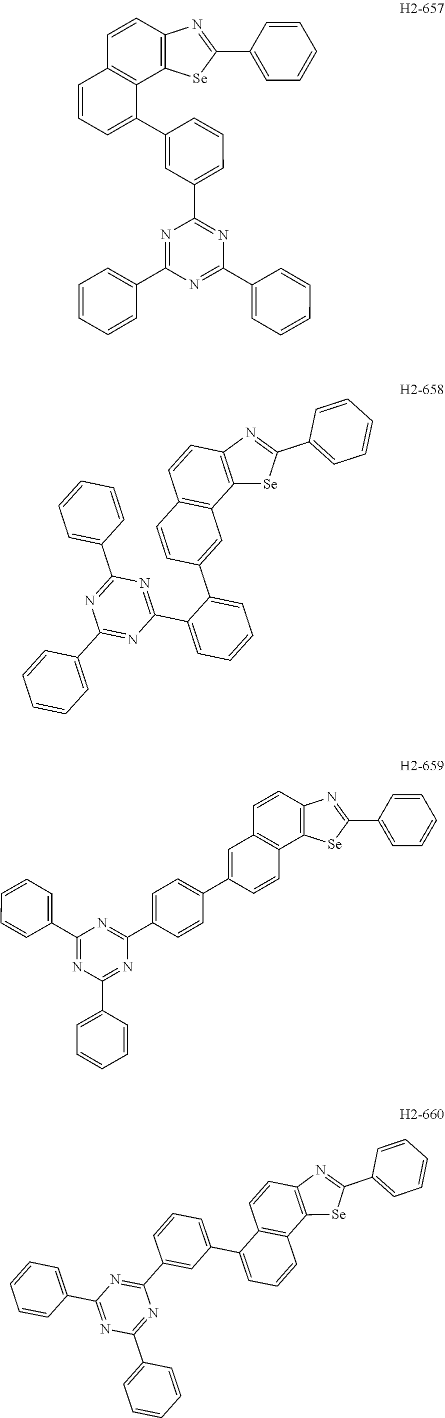 [SMILES: c1ccc(-c2nc(-c3ccccc3)nc(-c3cccc(-c4cccc5ccc6nc(-c7ccccc7)[se]c6c45)c3)n2)cc1 ; c1ccc(-c2nc(-c3ccccc3)nc(-c3ccccc3-c3ccc4ccc5nc(-c6ccccc6)[se]c5c4c3)n2)cc1 ; c1ccc(-c2nc(-c3ccccc3)nc(-c3ccc(-c4ccc5c(ccc6nc(-c7ccccc7)[se]c65)c4)cc3)n2)cc1 ; c1ccc(-c2nc(-c3ccccc3)nc(-c3cccc(-c4cccc5c4ccc4nc(-c6ccccc6)[se]c45)c3)n2)cc1]

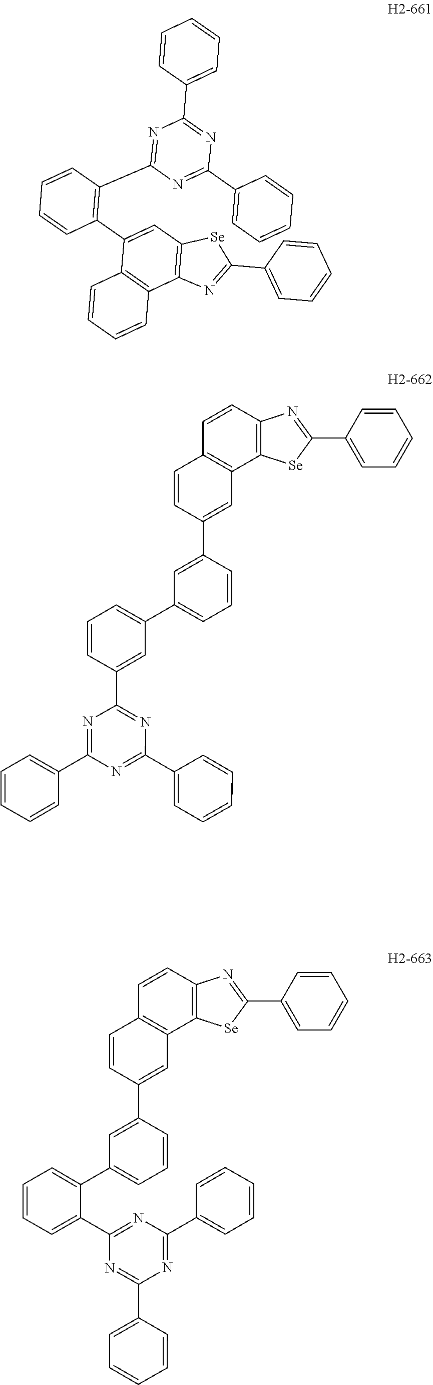 [SMILES: c1ccc(-c2nc(-c3ccccc3)nc(-c3ccccc3-c3cc4[se]c(-c5ccccc5)nc4c4ccccc34)n2)cc1 ; c1ccc(-c2nc(-c3ccccc3)nc(-c3cccc(-c4cccc(-c5ccc6ccc7nc(-c8ccccc8)[se]c7c6c5)c4)c3)n2)cc1 ; c1ccc(-c2nc(-c3ccccc3)nc(-c3ccccc3-c3cccc(-c4ccc5ccc6nc(-c7ccccc7)[se]c6c5c4)c3)n2)cc1]

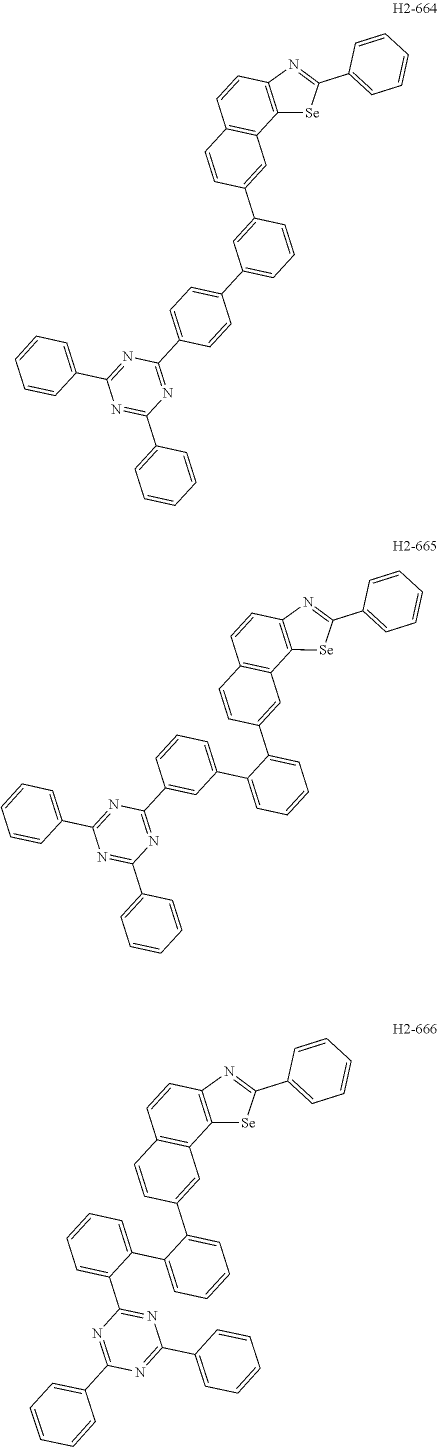 [SMILES: c1ccc(-c2nc(-c3ccccc3)nc(-c3ccc(-c4cccc(-c5ccc6ccc7nc(-c8ccccc8)[se]c7c6c5)c4)cc3)n2)cc1 ; c1ccc(-c2nc(-c3ccccc3)nc(-c3cccc(-c4ccccc4-c4ccc5ccc6nc(-c7ccccc7)[se]c6c5c4)c3)n2)cc1 ; c1ccc(-c2nc(-c3ccccc3)nc(-c3ccccc3-c3ccccc3-c3ccc4ccc5nc(-c6ccccc6)[se]c5c4c3)n2)cc1]

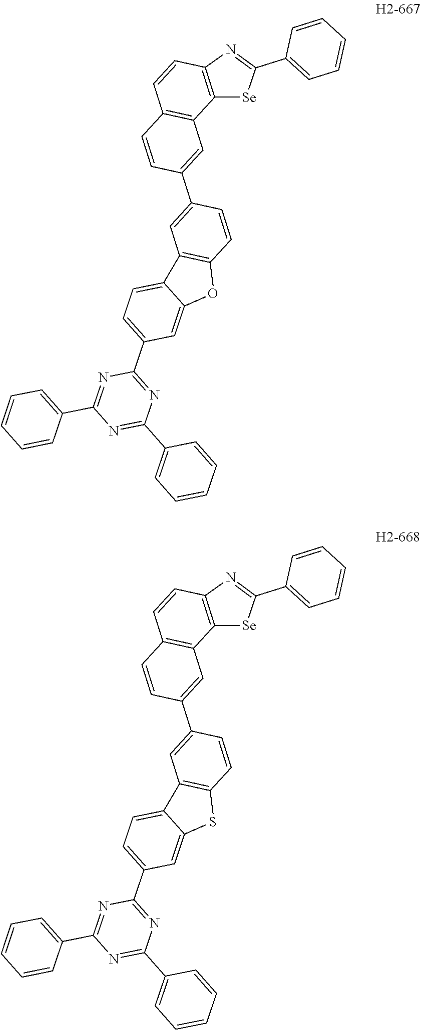 [SMILES: c1ccc(-c2nc(-c3ccccc3)nc(-c3ccc4c(c3)oc3ccc(-c5ccc6ccc7nc(-c8ccccc8)[se]c7c6c5)cc34)n2)cc1 ; c1ccc(-c2nc(-c3ccccc3)nc(-c3ccc4c(c3)sc3ccc(-c5ccc6ccc7nc(-c8ccccc8)[se]c7c6c5)cc34)n2)cc1]

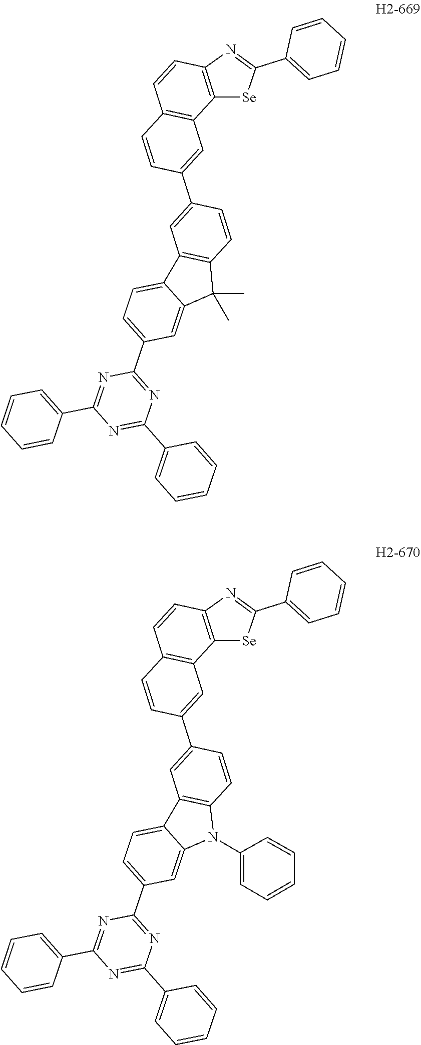 [SMILES: CC1(C)c2ccc(-c3ccc4ccc5nc(-c6ccccc6)[se]c5c4c3)cc2-c2ccc(-c3nc(-c4ccccc4)nc(-c4ccccc4)n3)cc21 ; c1ccc(-c2nc(-c3ccccc3)nc(-c3ccc4c5cc(-c6ccc7ccc8nc(-c9ccccc9)[se]c8c7c6)ccc5n(-c5ccccc5)c4c3)n2)cc1]

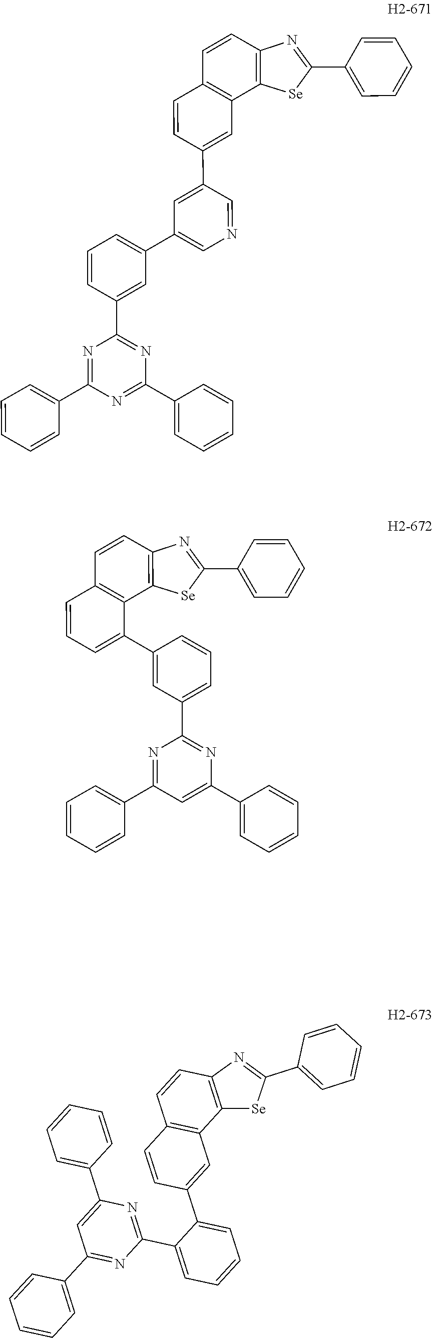 [SMILES: c1ccc(-c2nc(-c3ccccc3)nc(-c3cccc(-c4cncc(-c5ccc6ccc7nc(-c8ccccc8)[se]c7c6c5)c4)c3)n2)cc1 ; c1ccc(-c2cc(-c3ccccc3)nc(-c3cccc(-c4cccc5ccc6nc(-c7ccccc7)[se]c6c45)c3)n2)cc1 ; c1ccc(-c2cc(-c3ccccc3)nc(-c3ccccc3-c3ccc4ccc5nc(-c6ccccc6)[se]c5c4c3)n2)cc1]

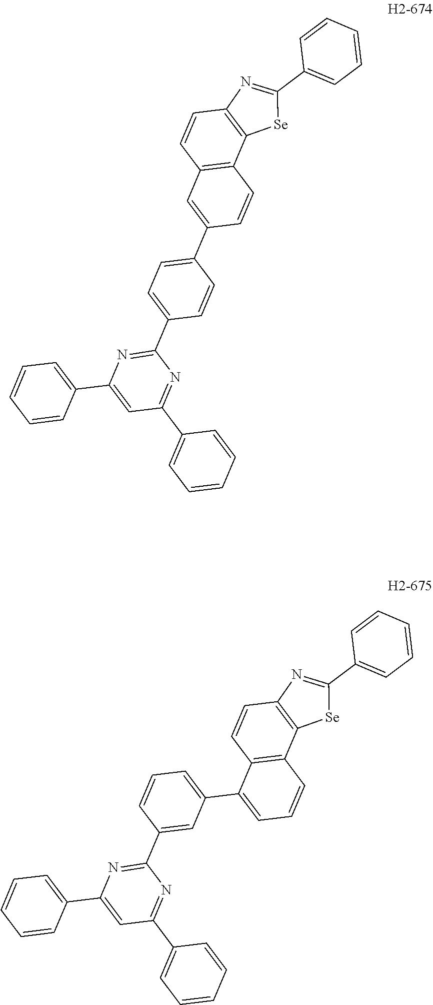 [SMILES: c1ccc(-c2cc(-c3ccccc3)nc(-c3ccc(-c4ccc5c(ccc6nc(-c7ccccc7)[se]c65)c4)cc3)n2)cc1 ; c1ccc(-c2cc(-c3ccccc3)nc(-c3cccc(-c4cccc5c4ccc4nc(-c6ccccc6)[se]c45)c3)n2)cc1]

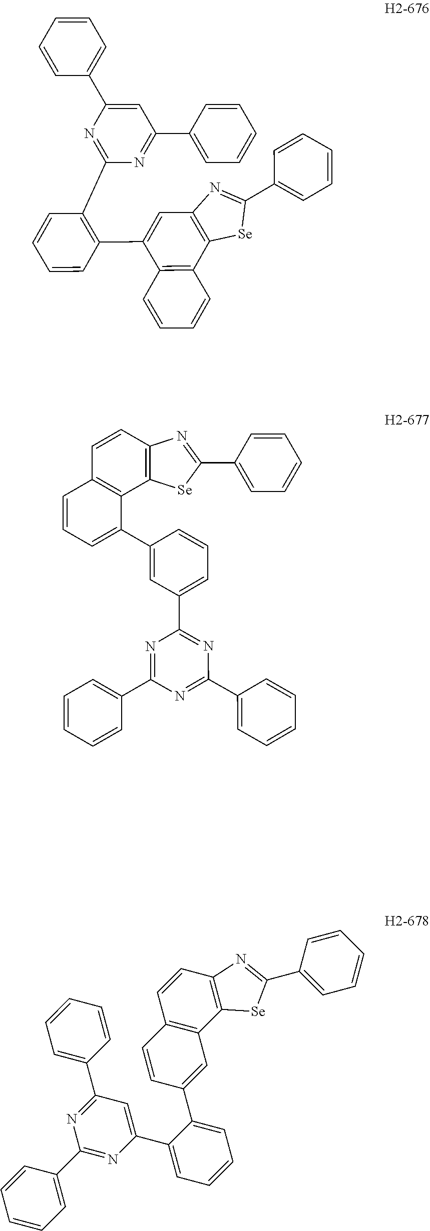 [SMILES: c1ccc(-c2cc(-c3ccccc3)nc(-c3ccccc3-c3cc4nc(-c5ccccc5)[se]c4c4ccccc34)n2)cc1 ; c1ccc(-c2nc(-c3ccccc3)nc(-c3cccc(-c4cccc5ccc6nc(-c7ccccc7)[se]c6c45)c3)n2)cc1 ; c1ccc(-c2cc(-c3ccccc3-c3ccc4ccc5nc(-c6ccccc6)[se]c5c4c3)nc(-c3ccccc3)n2)cc1]

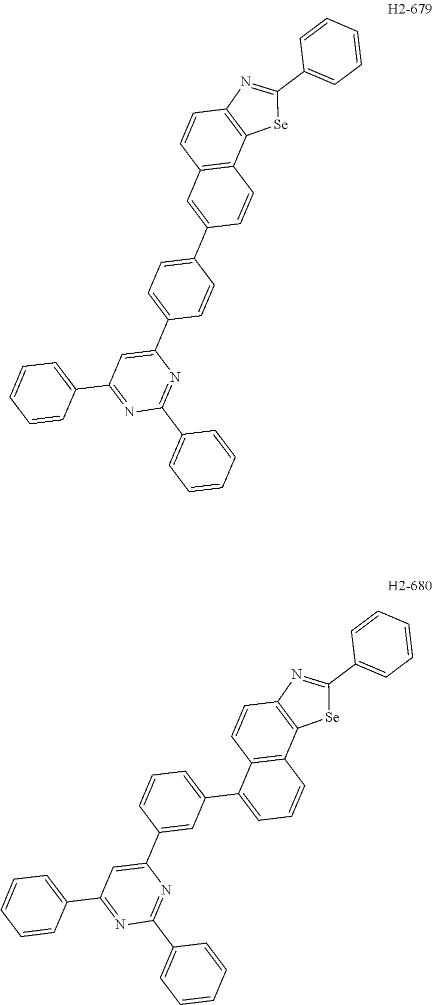 [SMILES: c1ccc(-c2cc(-c3ccc(-c4ccc5c(ccc6nc(-c7ccccc7)[se]c65)c4)cc3)nc(-c3ccccc3)n2)cc1 ; c1ccc(-c2cc(-c3cccc(-c4cccc5c4ccc4nc(-c6ccccc6)[se]c45)c3)nc(-c3ccccc3)n2)cc1]

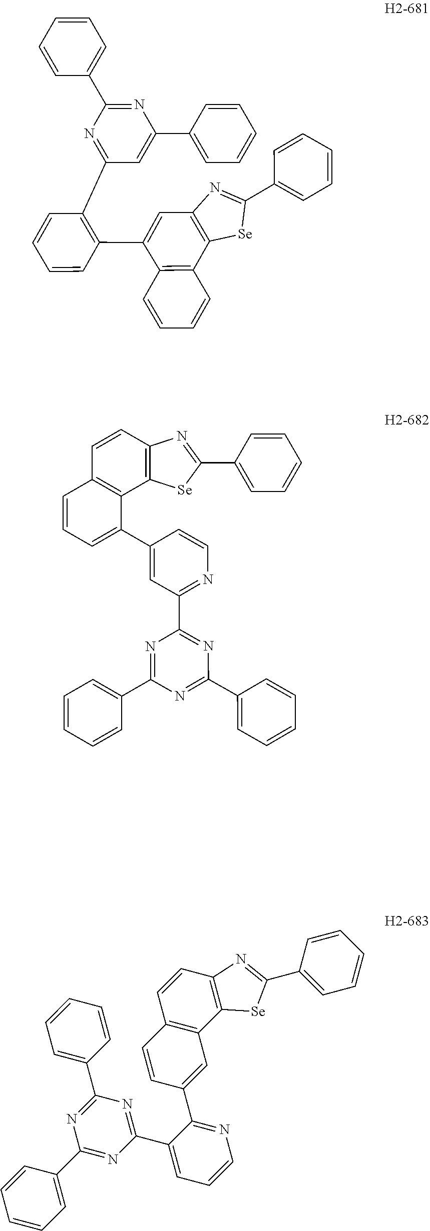 [SMILES: c1ccc(-c2cc(-c3ccccc3-c3cc4nc(-c5ccccc5)[se]c4c4ccccc34)nc(-c3ccccc3)n2)cc1 ; c1ccc(-c2nc(-c3ccccc3)nc(-c3cc(-c4cccc5ccc6nc(-c7ccccc7)[se]c6c45)ccn3)n2)cc1 ; c1ccc(-c2nc(-c3ccccc3)nc(-c3cccnc3-c3ccc4ccc5nc(-c6ccccc6)[se]c5c4c3)n2)cc1]

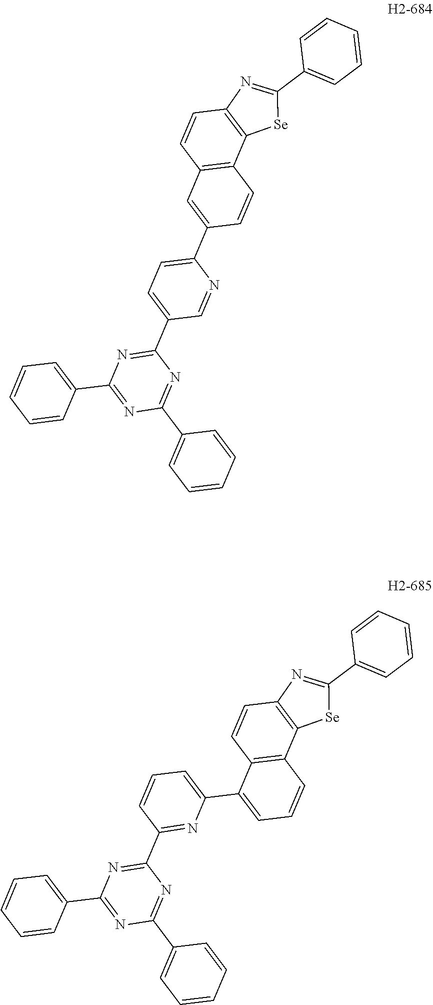 [SMILES: c1ccc(-c2nc(-c3ccccc3)nc(-c3ccc(-c4ccc5c(ccc6nc(-c7ccccc7)[se]c65)c4)nc3)n2)cc1 ; c1ccc(-c2nc(-c3ccccc3)nc(-c3cccc(-c4cccc5c4ccc4nc(-c6ccccc6)[se]c45)n3)n2)cc1]

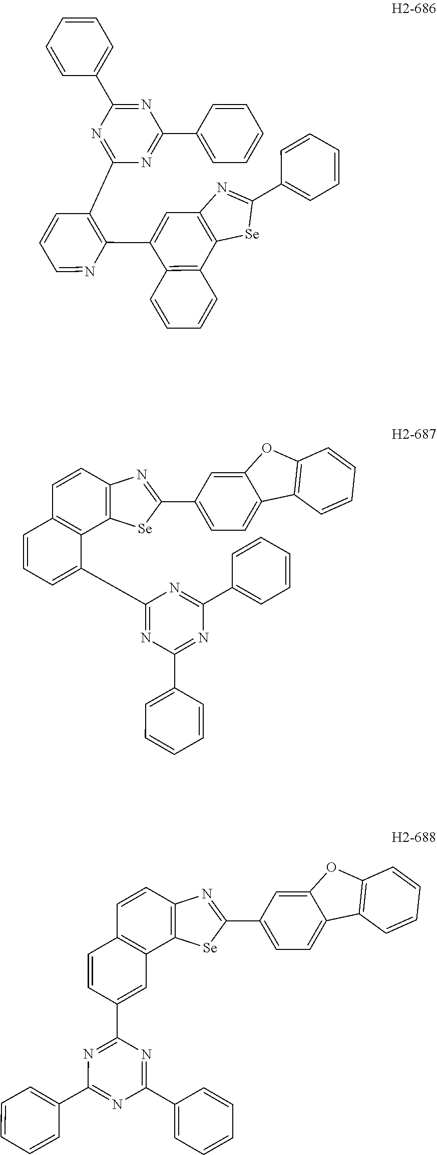 [SMILES: c1ccc(-c2nc(-c3ccccc3)nc(-c3cccnc3-c3cc4nc(-c5ccccc5)[se]c4c4ccccc34)n2)cc1 ; c1ccc(-c2nc(-c3ccccc3)nc(-c3cccc4ccc5nc(-c6ccc7c(c6)oc6ccccc67)[se]c5c34)n2)cc1 ; c1ccc(-c2nc(-c3ccccc3)nc(-c3ccc4ccc5nc(-c6ccc7c(c6)oc6ccccc67)[se]c5c4c3)n2)cc1]

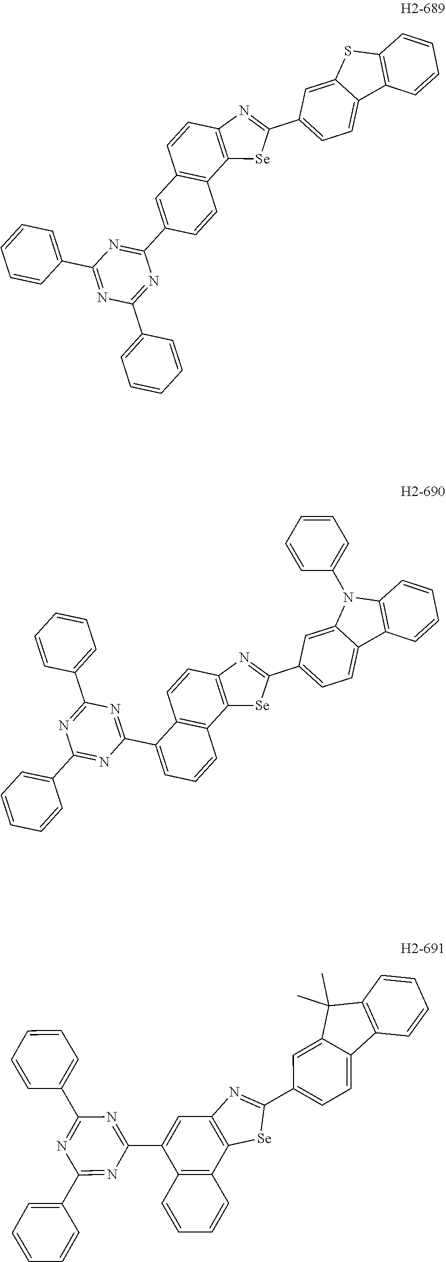 [SMILES: c1ccc(-c2nc(-c3ccccc3)nc(-c3ccc4c(ccc5nc(-c6ccc7c(c6)sc6ccccc67)[se]c54)c3)n2)cc1 ; c1ccc(-c2nc(-c3ccccc3)nc(-c3cccc4c3ccc3nc(-c5ccc6c7ccccc7n(-c7ccccc7)c6c5)[se]c34)n2)cc1 ; CC1(C)c2ccccc2-c2ccc(-c3nc4cc(-c5nc(-c6ccccc6)nc(-c6ccccc6)n5)c5ccccc5c4[se]3)cc21]

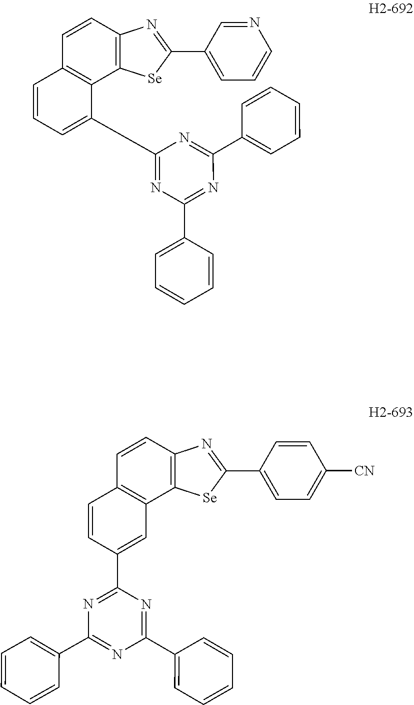 [SMILES: c1ccc(-c2nc(-c3ccccc3)nc(-c3cccc4ccc5nc(-c6cccnc6)[se]c5c34)n2)cc1 ; N#Cc1ccc(-c2nc3ccc4ccc(-c5nc(-c6ccccc6)nc(-c6ccccc6)n5)cc4c3[se]2)cc1]

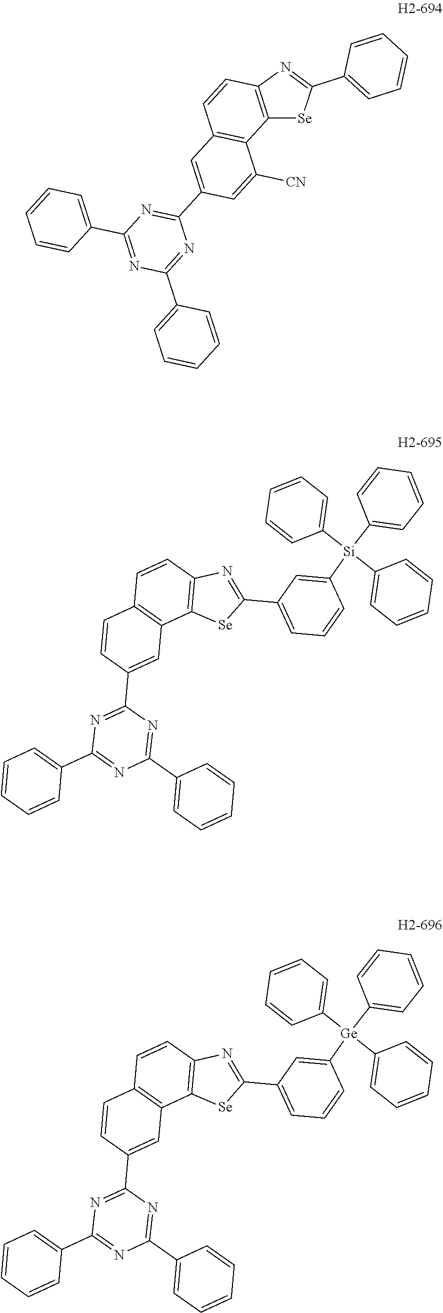

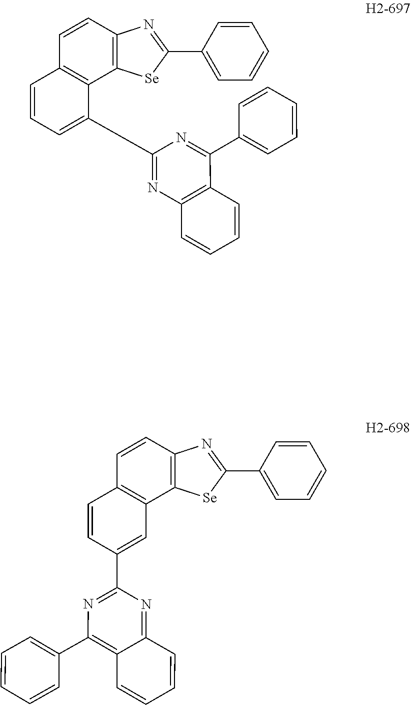 [SMILES: c1ccc(-c2nc3ccc4cccc(-c5nc(-c6ccccc6)c6ccccc6n5)c4c3[se]2)cc1 ; c1ccc(-c2nc3ccc4ccc(-c5nc(-c6ccccc6)c6ccccc6n5)cc4c3[se]2)cc1]

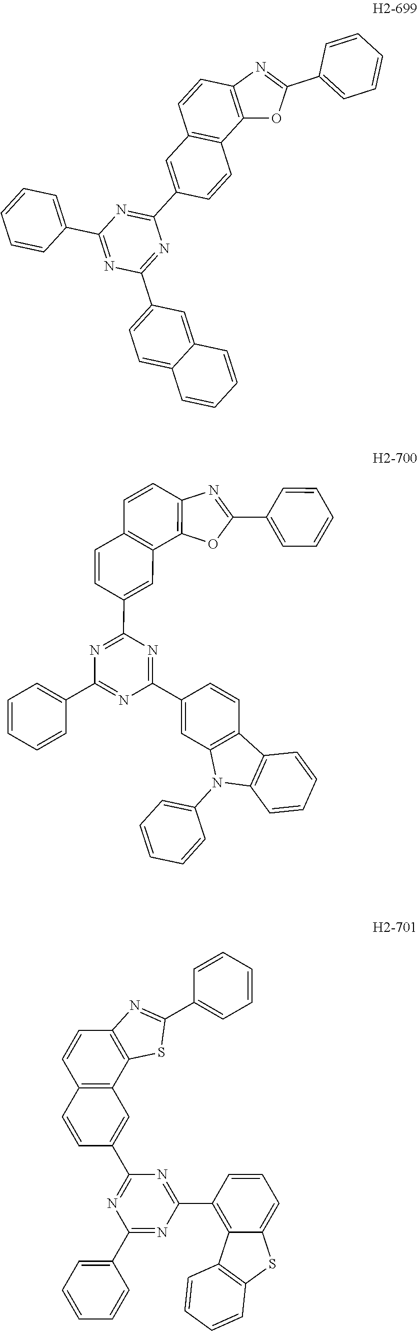 [SMILES: c1ccc(-c2nc(-c3ccc4ccccc4c3)nc(-c3ccc4c(ccc5nc(-c6ccccc6)oc54)c3)n2)cc1 ; c1ccc(-c2nc(-c3ccc4ccc5nc(-c6ccccc6)oc5c4c3)nc(-c3ccc4c5ccccc5n(-c5ccccc5)c4c3)n2)cc1 ; c1ccc(-c2nc(-c3ccc4ccc5nc(-c6ccccc6)sc5c4c3)nc(-c3cccc4sc5ccccc5c34)n2)cc1]

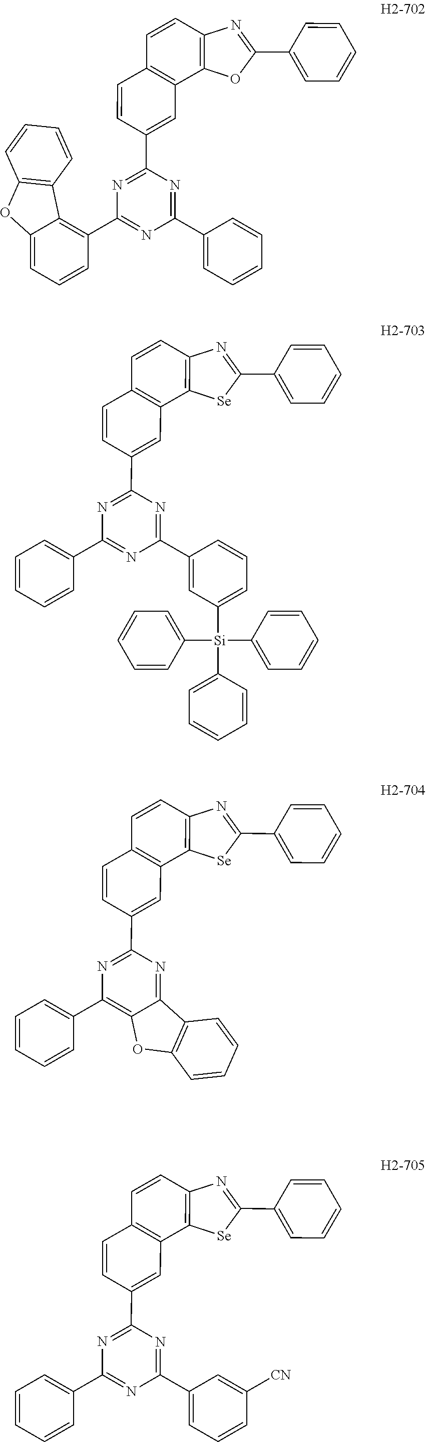 [SMILES: c1ccc(-c2nc(-c3ccc4ccc5nc(-c6ccccc6)oc5c4c3)nc(-c3cccc4oc5ccccc5c34)n2)cc1 ; c1ccc(-c2nc(-c3cccc([Si](c4ccccc4)(c4ccccc4)c4ccccc4)c3)nc(-c3ccc4ccc5nc(-c6ccccc6)[se]c5c4c3)n2)cc1 ; c1ccc(-c2nc3ccc4ccc(-c5nc(-c6ccccc6)c6oc7ccccc7c6n5)cc4c3[se]2)cc1 ; N#Cc1cccc(-c2nc(-c3ccccc3)nc(-c3ccc4ccc5nc(-c6ccccc6)[se]c5c4c3)n2)c1]

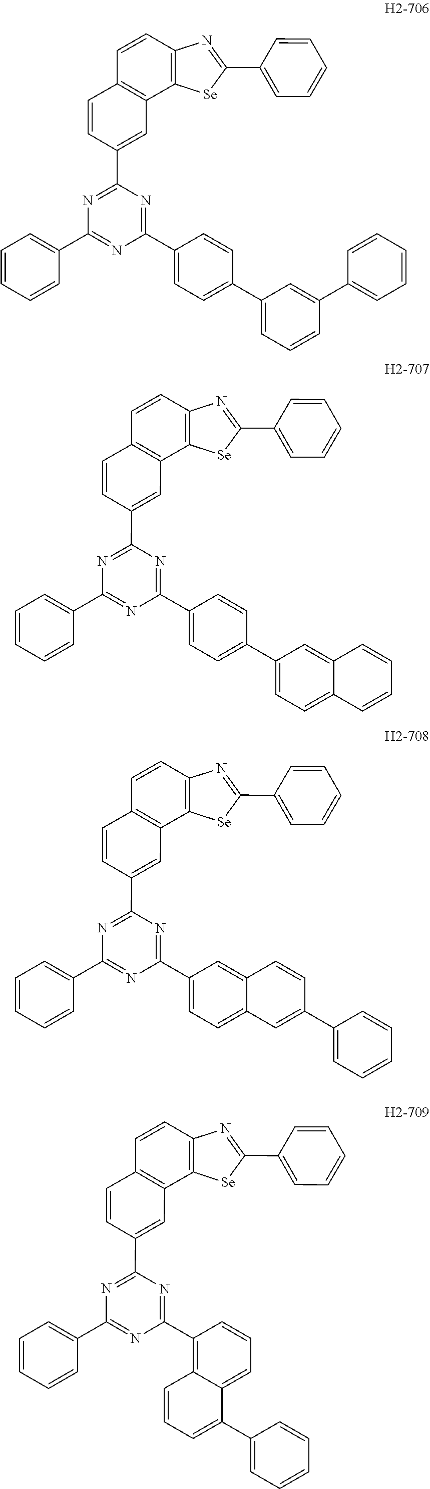 [SMILES: c1ccc(-c2cccc(-c3ccc(-c4nc(-c5ccccc5)nc(-c5ccc6ccc7nc(-c8ccccc8)[se]c7c6c5)n4)cc3)c2)cc1 ; c1ccc(-c2nc(-c3ccc(-c4ccc5ccccc5c4)cc3)nc(-c3ccc4ccc5nc(-c6ccccc6)[se]c5c4c3)n2)cc1 ; c1ccc(-c2ccc3cc(-c4nc(-c5ccccc5)nc(-c5ccc6ccc7nc(-c8ccccc8)[se]c7c6c5)n4)ccc3c2)cc1 ; c1ccc(-c2nc(-c3ccc4ccc5nc(-c6ccccc6)[se]c5c4c3)nc(-c3cccc4c(-c5ccccc5)cccc34)n2)cc1]

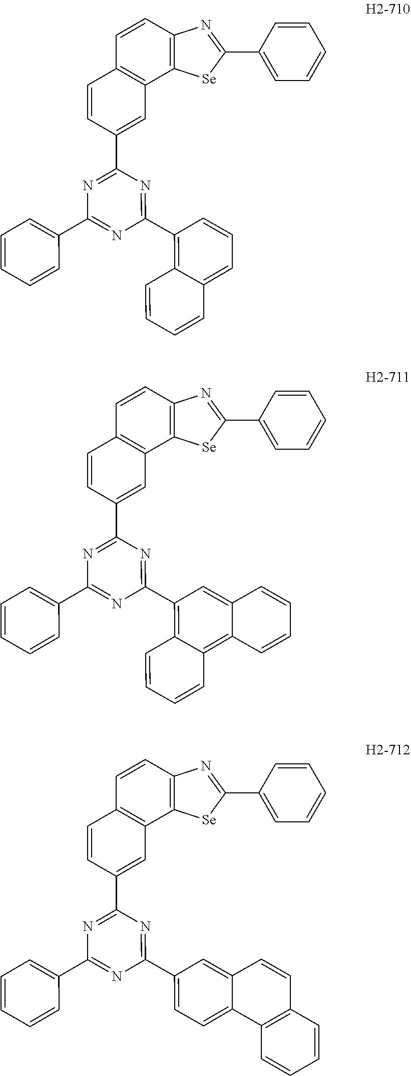 [SMILES: c1ccc(-c2nc(-c3ccc4ccc5nc(-c6ccccc6)[se]c5c4c3)nc(-c3cccc4ccccc34)n2)cc1 ; c1ccc(-c2nc(-c3ccc4ccc5nc(-c6ccccc6)[se]c5c4c3)nc(-c3cc4ccccc4c4ccccc34)n2)cc1 ; c1ccc(-c2nc(-c3ccc4c(ccc5ccccc54)c3)nc(-c3ccc4ccc5nc(-c6ccccc6)[se]c5c4c3)n2)cc1]

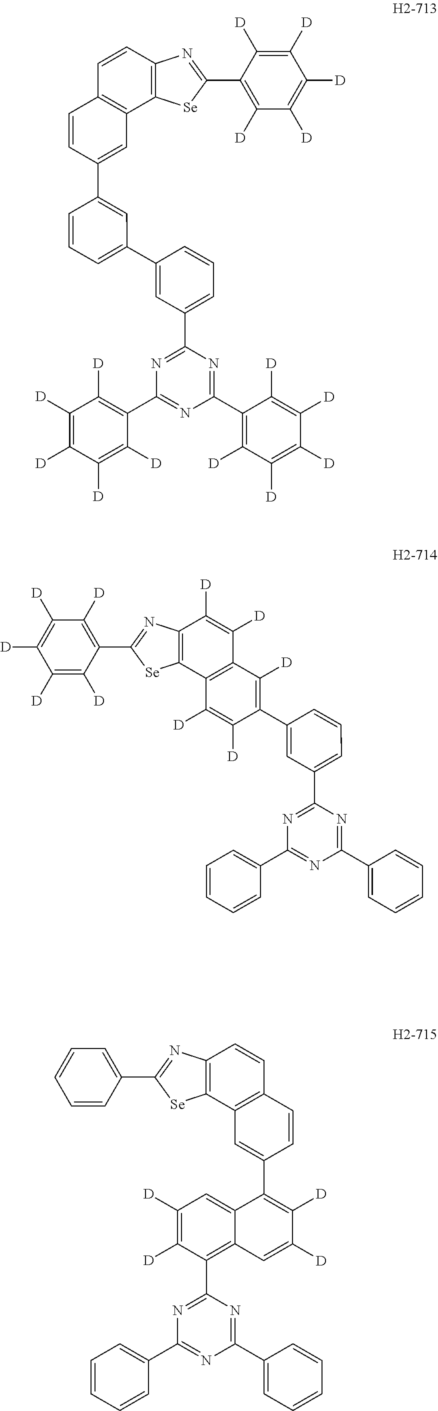 [SMILES: [2H]c1c([2H])c([2H])c(-c2nc(-c3cccc(-c4cccc(-c5ccc6ccc7nc(-c8c([2H])c([2H])c([2H])c([2H])c8[2H])[se]c7c6c5)c4)c3)nc(-c3c([2H])c([2H])c([2H])c([2H])c3[2H])n2)c([2H])c1[2H] ; [2H]c1c([2H])c([2H])c(-c2nc3c([2H])c([2H])c4c([2H])c(-c5cccc(-c6nc(-c7ccccc7)nc(-c7ccccc7)n6)c5)c([2H])c([2H])c4c3[se]2)c([2H])c1[2H] ; [2H]c1cc2c(-c3nc(-c4ccccc4)nc(-c4ccccc4)n3)c([2H])c([2H])cc2c(-c2ccc3ccc4nc(-c5ccccc5)[se]c4c3c2)c1[2H]]

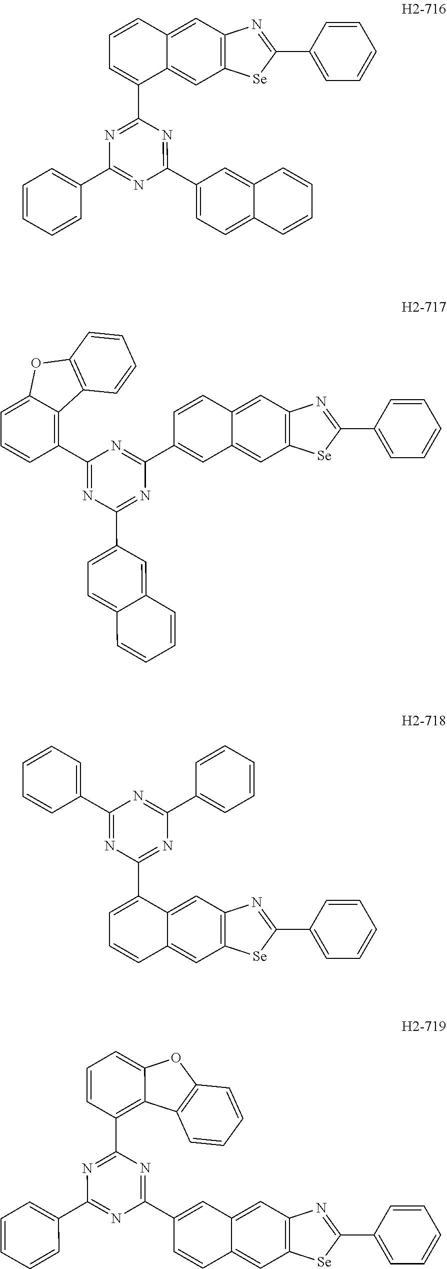 [SMILES: c1ccc(-c2nc(-c3ccc4ccccc4c3)nc(-c3cccc4cc5nc(-c6ccccc6)[se]c5cc34)n2)cc1 ; c1ccc(-c2nc3cc4ccc(-c5nc(-c6ccc7ccccc7c6)nc(-c6cccc7oc8ccccc8c67)n5)cc4cc3[se]2)cc1 ; c1ccc(-c2nc(-c3ccccc3)nc(-c3cccc4cc5[se]c(-c6ccccc6)nc5cc34)n2)cc1 ; c1ccc(-c2nc(-c3ccc4cc5[se]c(-c6ccccc6)nc5cc4c3)nc(-c3cccc4oc5ccccc5c34)n2)cc1]

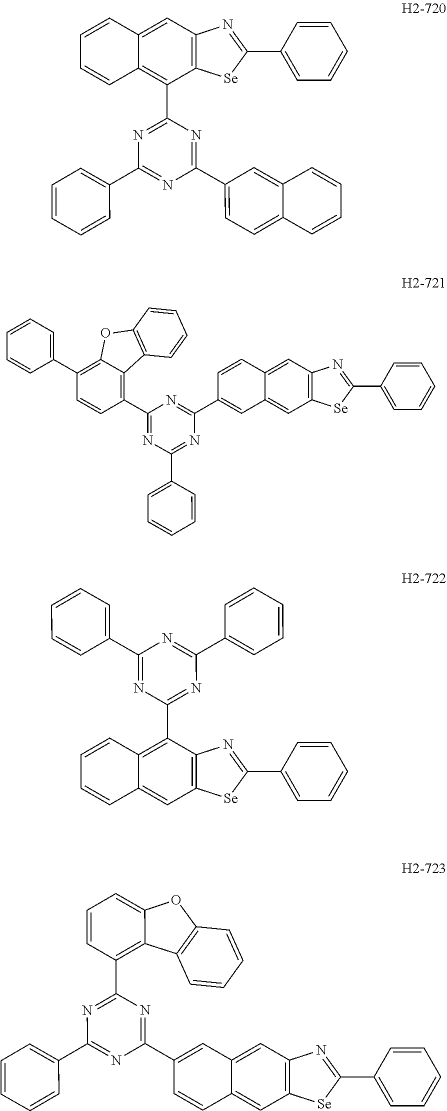 [SMILES: c1ccc(-c2nc(-c3ccc4ccccc4c3)nc(-c3c4ccccc4cc4nc(-c5ccccc5)[se]c34)n2)cc1 ; c1ccc(-c2nc(-c3ccc4cc5nc(-c6ccccc6)[se]c5cc4c3)nc(-c3ccc(-c4ccccc4)c4oc5ccccc5c34)n2)cc1 ; c1ccc(-c2nc(-c3ccccc3)nc(-c3c4ccccc4cc4[se]c(-c5ccccc5)nc34)n2)cc1 ; c1ccc(-c2nc(-c3ccc4cc5[se]c(-c6ccccc6)nc5cc4c3)nc(-c3cccc4oc5ccccc5c34)n2)cc1]

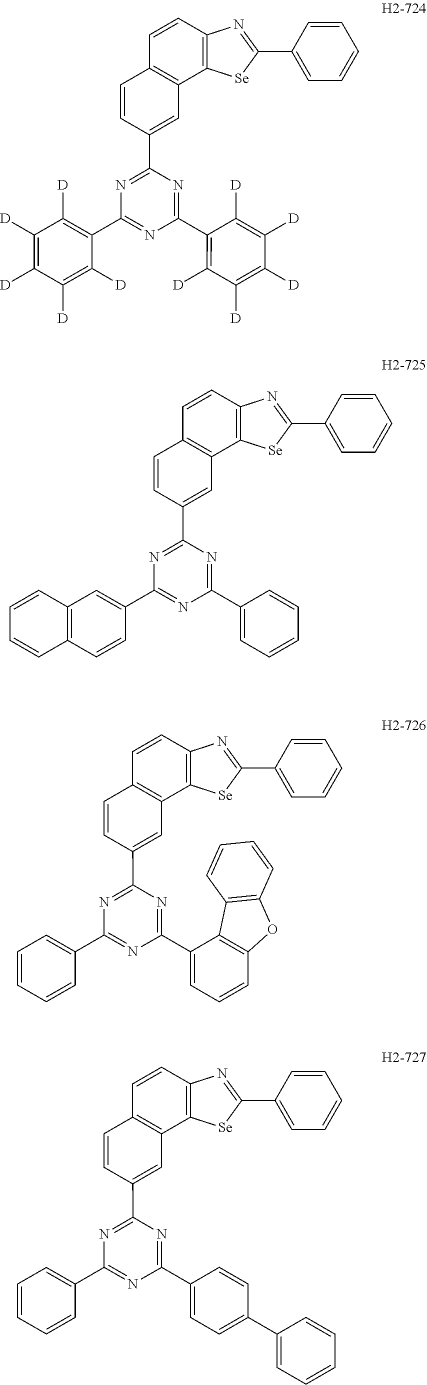 [SMILES: [2H]c1c([2H])c([2H])c(-c2nc(-c3ccc4ccc5nc(-c6ccccc6)[se]c5c4c3)nc(-c3c([2H])c([2H])c([2H])c([2H])c3[2H])n2)c([2H])c1[2H] ; c1ccc(-c2nc(-c3ccc4ccccc4c3)nc(-c3ccc4ccc5nc(-c6ccccc6)[se]c5c4c3)n2)cc1 ; c1ccc(-c2nc(-c3ccc4ccc5nc(-c6ccccc6)[se]c5c4c3)nc(-c3cccc4oc5ccccc5c34)n2)cc1 ; c1ccc(-c2ccc(-c3nc(-c4ccccc4)nc(-c4ccc5ccc6nc(-c7ccccc7)[se]c6c5c4)n3)cc2)cc1]

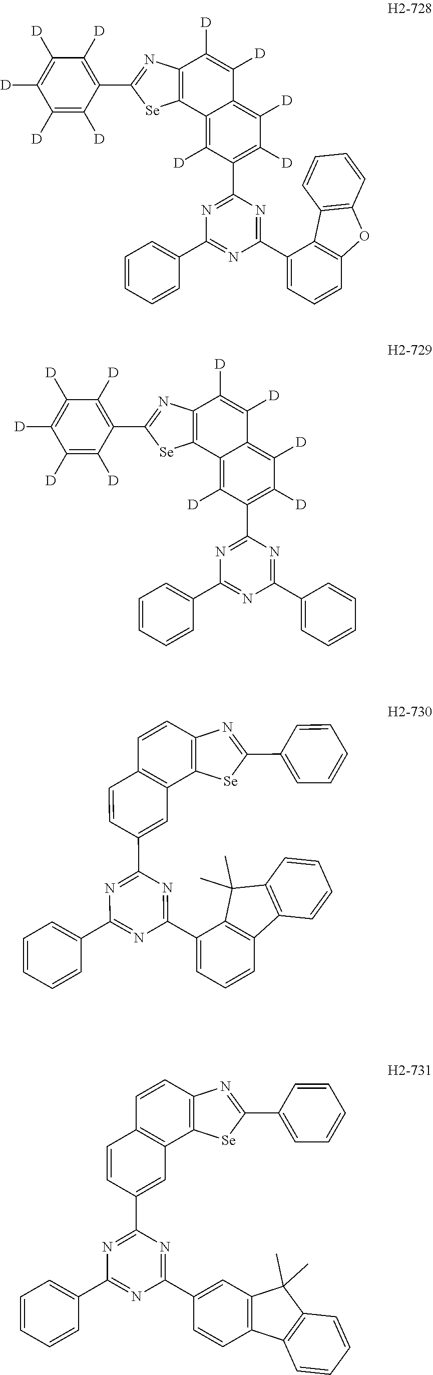 [SMILES: [2H]c1c([2H])c([2H])c(-c2nc3c([2H])c([2H])c4c([2H])c([2H])c(-c5nc(-c6ccccc6)nc(-c6cccc7oc8ccccc8c67)n5)c([2H])c4c3[se]2)c([2H])c1[2H] ; [2H]c1c([2H])c([2H])c(-c2nc3c([2H])c([2H])c4c([2H])c([2H])c(-c5nc(-c6ccccc6)nc(-c6ccccc6)n5)c([2H])c4c3[se]2)c([2H])c1[2H] ; CC1(C)c2ccccc2-c2cccc(-c3nc(-c4ccccc4)nc(-c4ccc5ccc6nc(-c7ccccc7)[se]c6c5c4)n3)c21 ; CC1(C)c2ccccc2-c2ccc(-c3nc(-c4ccccc4)nc(-c4ccc5ccc6nc(-c7ccccc7)[se]c6c5c4)n3)cc21]

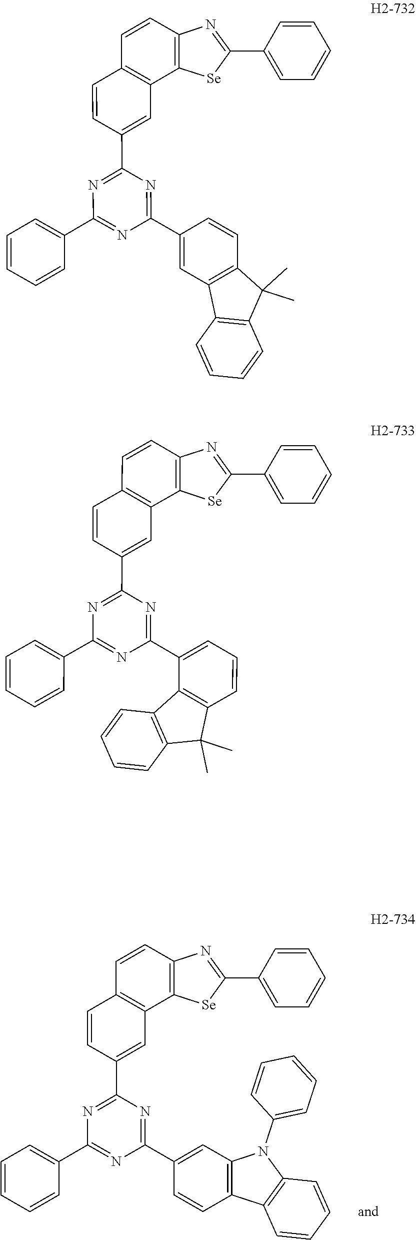 [SMILES: CC1(C)c2ccccc2-c2cc(-c3nc(-c4ccccc4)nc(-c4ccc5ccc6nc(-c7ccccc7)[se]c6c5c4)n3)ccc21 ; CC1(C)c2ccccc2-c2c(-c3nc(-c4ccccc4)nc(-c4ccc5ccc6nc(-c7ccccc7)[se]c6c5c4)n3)cccc21 ; c1ccc(-c2nc(-c3ccc4ccc5nc(-c6ccccc6)[se]c5c4c3)nc(-c3ccc4c5ccccc5n(-c5ccccc5)c4c3)n2)cc1]

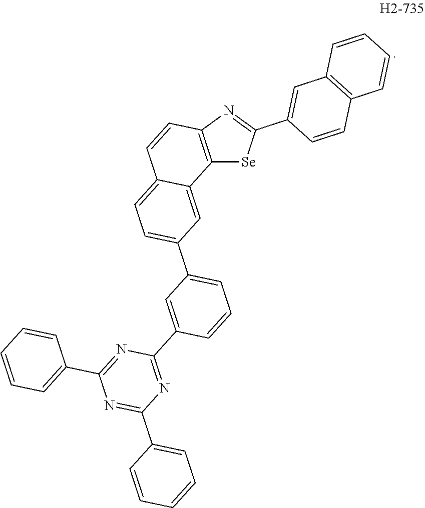 [SMILES: c1ccc(-c2nc(-c3ccccc3)nc(-c3cccc(-c4ccc5ccc6nc(-c7ccc8ccccc8c7)[se]c6c5c4)c3)n2)cc1]

7. An organic electroluminescent device comprising an anode, a cathode, and at least one light-emitting layer between the anode and the cathode, wherein the at least one light-emitting layer comprises the plurality of host materials according to claim 1.

8. An organic electroluminescent compound represented by the following formula 1′: 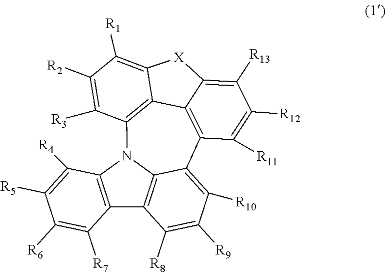
in formula 1′,
X represents —N(R)—, —C(R′)(R″)— or —Se—;
R, R′, and R″, each independently, represent a substituted or unsubstituted (C1-C30)alkyl, a substituted or unsubstituted (C6-C30)aryl, a substituted or unsubstituted (3- to 30-membered)heteroaryl, -L₁-N—(Ar₁)(Ar₂) or -L₂-N(Ar₃)-L₃-N—(Ar₄)(Ar₅); or R′ and R″ may be linked to each other to form a ring(s), and R′ and R″ may be the same or different;
R₁ to R₁₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), -L₁-N—(Ar₁)(Ar₂) or -L₂-N(Ar₃)-L₃-N—(Ar₄)(Ar₅); or may be linked to an adjacent substituent(s) to form a ring(s);
with the proviso that at least one of R, R′, R″, and R₁ to R₁₃ represents -L₁-N—(Ar₁)(Ar₂) or -L₂-N(Ar₃)-L₃-N—(Ar₄)(Ar₅);
L₁ and L₂, each independently, represent a single bond, a substituted or unsubstituted (C6-C30)arylene, or a substituted or unsubstituted (3- to 30-membered)heteroarylene;
L₃ represents a substituted or unsubstituted (C6-C30)arylene, or a substituted or unsubstituted (3- to 30-membered)heteroarylene; and
Ar₁ to Ar₅, each independently, represent a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl.

9. The organic electroluminescent compound according to claim 8, wherein the organic electroluminescent compound represented by formula 1′ is selected from the group consisting of the following compounds:

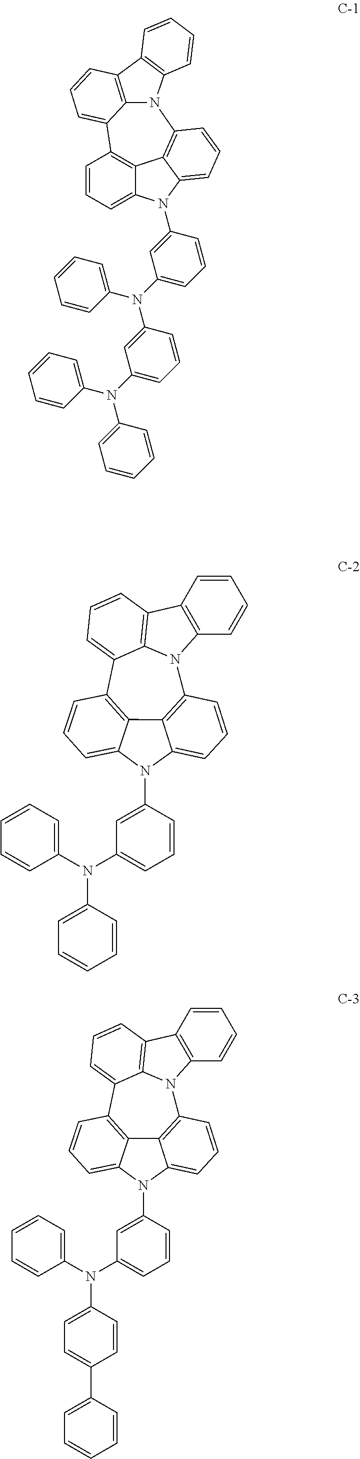 [SMILES: c1ccc(N(c2ccccc2)c2cccc(N(c3ccccc3)c3cccc(-n4c5cccc6c7cccc8c9ccccc9n(c9cccc4c9c65)c78)c3)c2)cc1 ; c1ccc(N(c2ccccc2)c2cccc(-n3c4cccc5c6cccc7c8ccccc8n(c8cccc3c8c54)c67)c2)cc1 ; c1ccc(-c2ccc(N(c3ccccc3)c3cccc(-n4c5cccc6c7cccc8c9ccccc9n(c9cccc4c9c65)c78)c3)cc2)cc1]

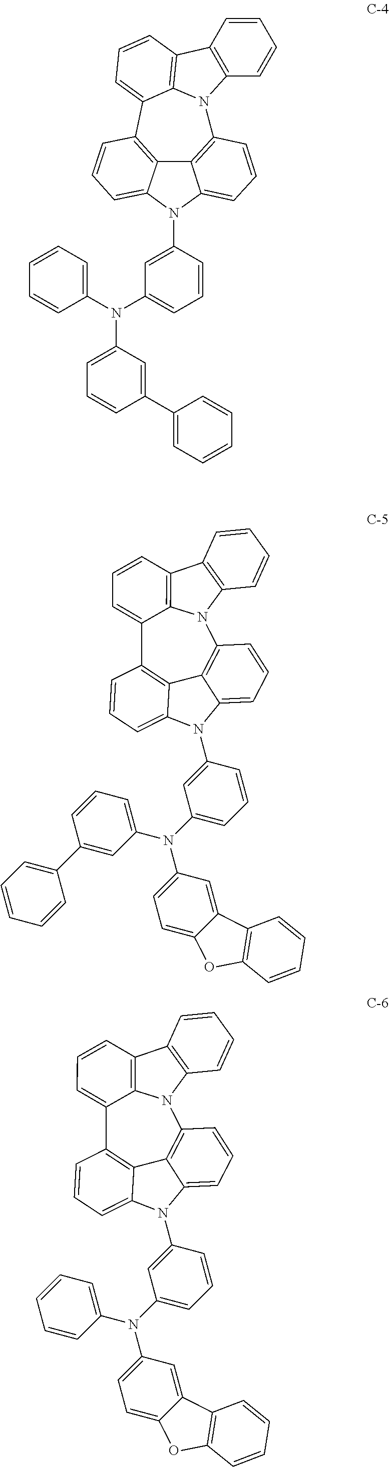

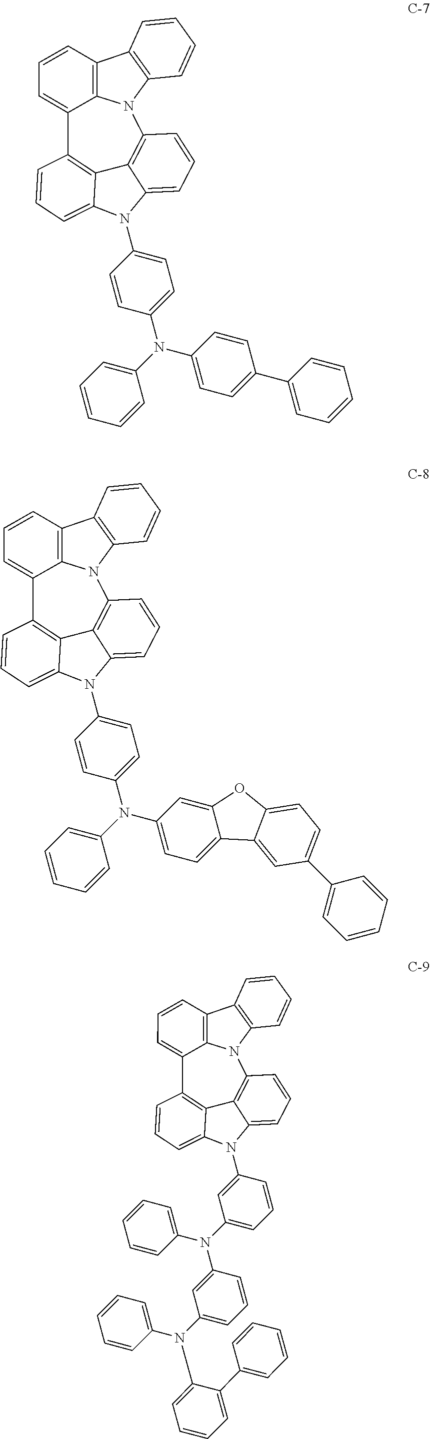 [SMILES: c1ccc(-c2ccc(N(c3ccccc3)c3ccc(-n4c5cccc6c7cccc8c9ccccc9n(c9cccc4c9c65)c78)cc3)cc2)cc1 ; c1ccc(-c2ccc3oc4cc(N(c5ccccc5)c5ccc(-n6c7cccc8c9cccc%10c%11ccccc%11n(c%11cccc6c%11c87)c9%10)cc5)ccc4c3c2)cc1 ; c1ccc(-c2ccccc2N(c2ccccc2)c2cccc(N(c3ccccc3)c3cccc(-n4c5cccc6c7cccc8c9ccccc9n(c9cccc4c9c65)c78)c3)c2)cc1]

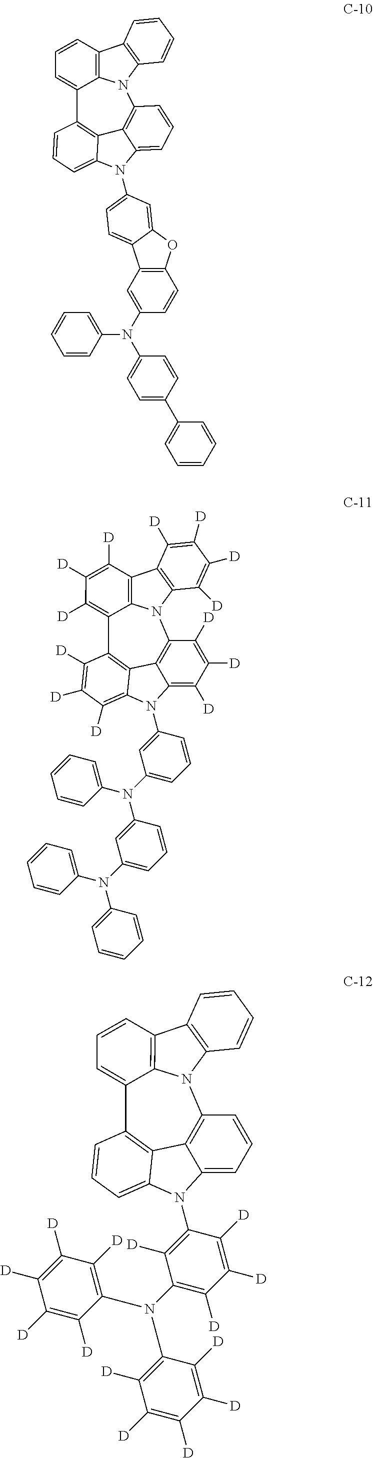 [SMILES: c1ccc(-c2ccc(N(c3ccccc3)c3ccc4oc5cc(-n6c7cccc8c9cccc%10c%11ccccc%11n(c%11cccc6c%11c87)c9%10)ccc5c4c3)cc2)cc1 ; [2H]c1c([2H])c([2H])c2c(c1[2H])c1c([2H])c([2H])c([2H])c3c4c([2H])c([2H])c([2H])c5c4c4c(c([2H])c([2H])c([2H])c4n2c31)n5-c1cccc(N(c2ccccc2)c2cccc(N(c3ccccc3)c3ccccc3)c2)c1 ; [2H]c1c([2H])c([2H])c(N(c2c([2H])c([2H])c([2H])c([2H])c2[2H])c2c([2H])c([2H])c([2H])c(-n3c4cccc5c6cccc7c8ccccc8n(c8cccc3c8c54)c67)c2[2H])c([2H])c1[2H]]

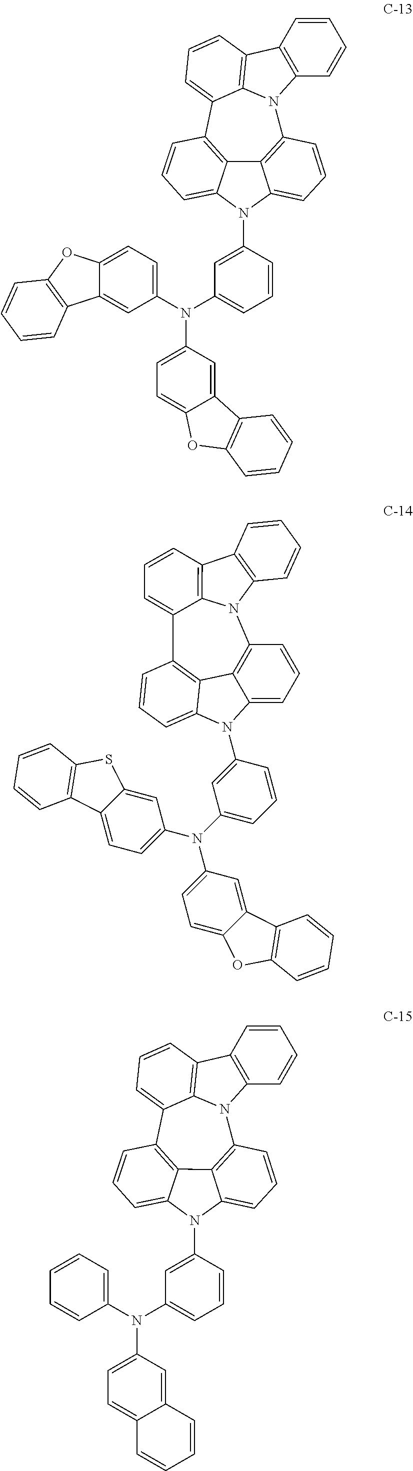 [SMILES: c1cc(N(c2ccc3oc4ccccc4c3c2)c2ccc3oc4ccccc4c3c2)cc(-n2c3cccc4c5cccc6c7ccccc7n(c7cccc2c7c43)c56)c1 ; c1cc(N(c2ccc3c(c2)sc2ccccc23)c2ccc3oc4ccccc4c3c2)cc(-n2c3cccc4c5cccc6c7ccccc7n(c7cccc2c7c43)c56)c1 ; c1ccc(N(c2cccc(-n3c4cccc5c6cccc7c8ccccc8n(c8cccc3c8c54)c67)c2)c2ccc3ccccc3c2)cc1]

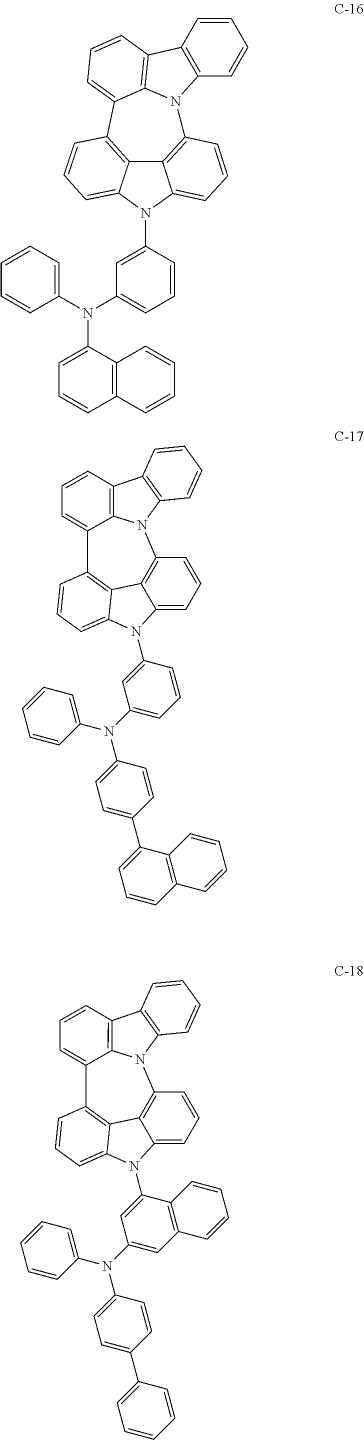

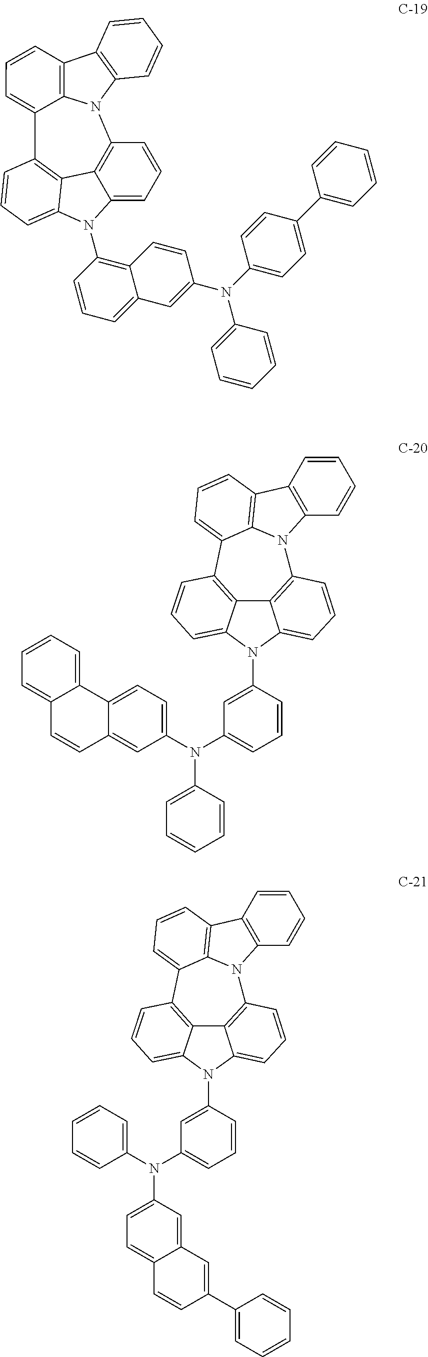 [SMILES: c1ccc(-c2ccc(N(c3ccccc3)c3ccc4c(-n5c6cccc7c8cccc9c%10ccccc%10n(c%10cccc5c%10c76)c89)cccc4c3)cc2)cc1 ; c1ccc(N(c2cccc(-n3c4cccc5c6cccc7c8ccccc8n(c8cccc3c8c54)c67)c2)c2ccc3c(ccc4ccccc43)c2)cc1 ; c1ccc(-c2ccc3ccc(N(c4ccccc4)c4cccc(-n5c6cccc7c8cccc9c%10ccccc%10n(c%10cccc5c%10c76)c89)c4)cc3c2)cc1]

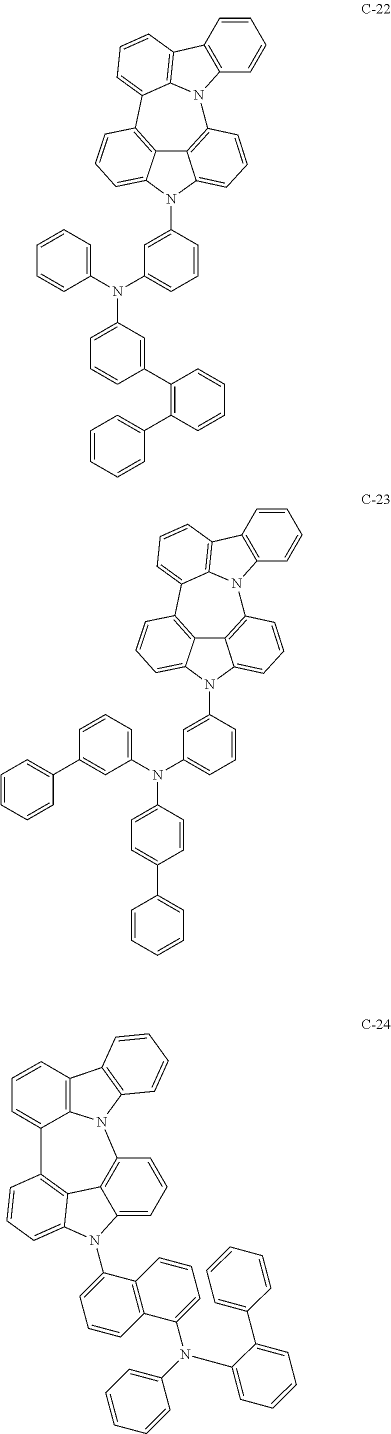 [SMILES: c1ccc(-c2ccccc2-c2cccc(N(c3ccccc3)c3cccc(-n4c5cccc6c7cccc8c9ccccc9n(c9cccc4c9c65)c78)c3)c2)cc1 ; c1ccc(-c2ccc(N(c3cccc(-c4ccccc4)c3)c3cccc(-n4c5cccc6c7cccc8c9ccccc9n(c9cccc4c9c65)c78)c3)cc2)cc1 ; c1ccc(-c2ccccc2N(c2ccccc2)c2cccc3c(-n4c5cccc6c7cccc8c9ccccc9n(c9cccc4c9c65)c78)cccc23)cc1]

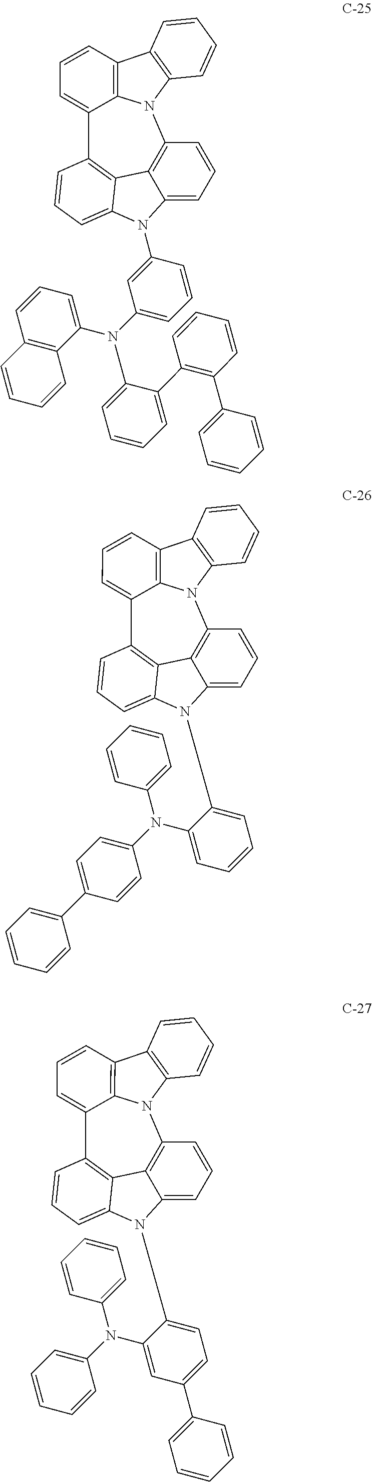

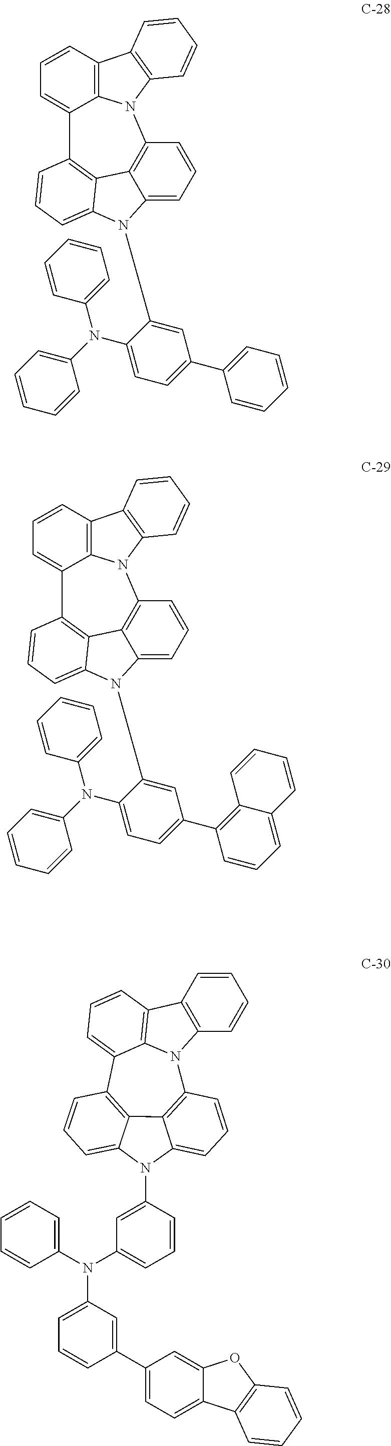 [SMILES: c1ccc(-c2ccc(N(c3ccccc3)c3ccccc3)c(-n3c4cccc5c6cccc7c8ccccc8n(c8cccc3c8c54)c67)c2)cc1 ; c1ccc(N(c2ccccc2)c2ccc(-c3cccc4ccccc34)cc2-n2c3cccc4c5cccc6c7ccccc7n(c7cccc2c7c43)c56)cc1 ; c1ccc(N(c2cccc(-c3ccc4c(c3)oc3ccccc34)c2)c2cccc(-n3c4cccc5c6cccc7c8ccccc8n(c8cccc3c8c54)c67)c2)cc1]

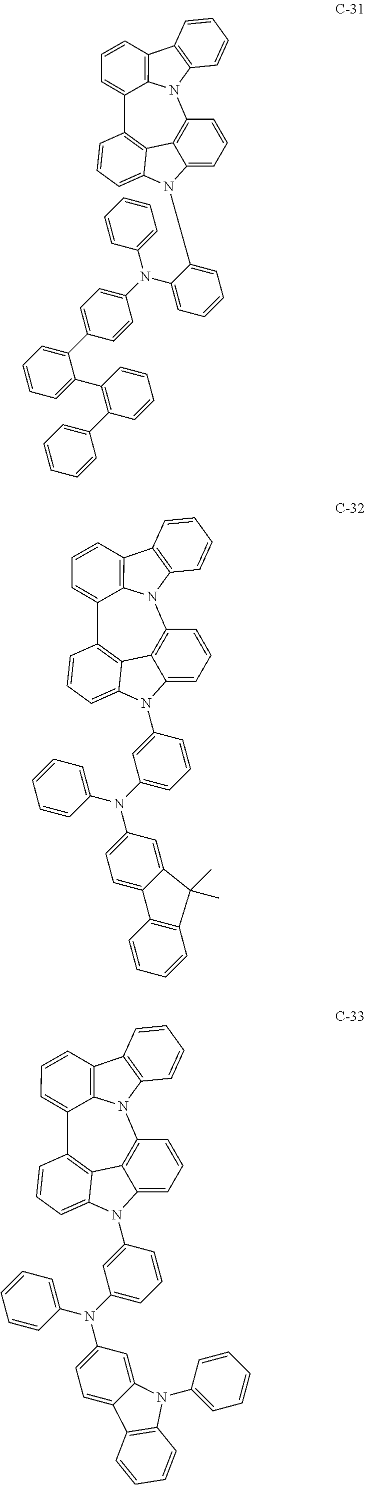 [SMILES: c1ccc(-c2ccccc2-c2ccccc2-c2ccc(N(c3ccccc3)c3ccccc3-n3c4cccc5c6cccc7c8ccccc8n(c8cccc3c8c54)c67)cc2)cc1 ; CC1(C)c2ccccc2-c2ccc(N(c3ccccc3)c3cccc(-n4c5cccc6c7cccc8c9ccccc9n(c9cccc4c9c65)c78)c3)cc21 ; c1ccc(N(c2cccc(-n3c4cccc5c6cccc7c8ccccc8n(c8cccc3c8c54)c67)c2)c2ccc3c4ccccc4n(-c4ccccc4)c3c2)cc1]

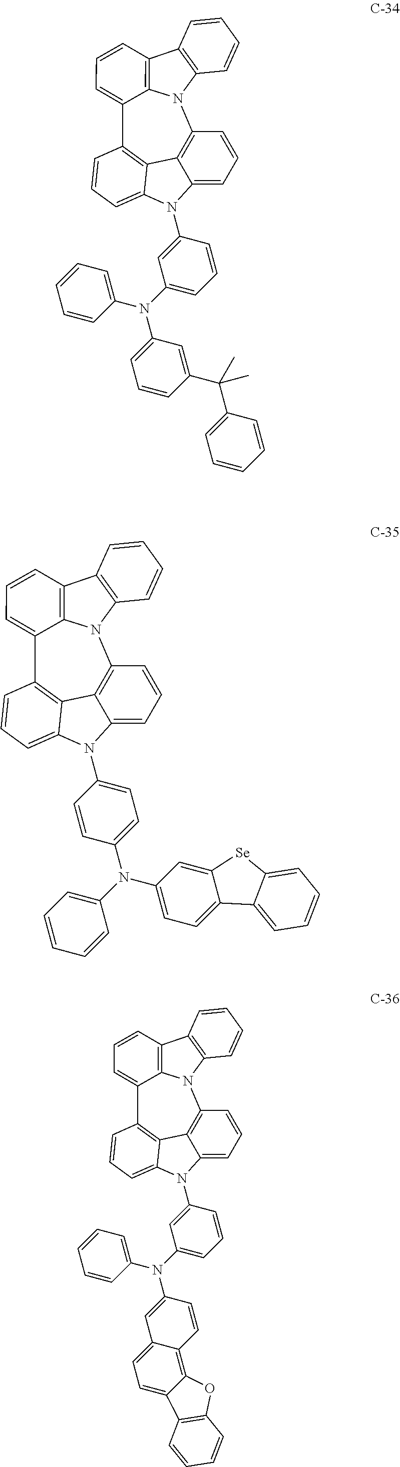 [SMILES: CC(C)(c1ccccc1)c1cccc(N(c2ccccc2)c2cccc(-n3c4cccc5c6cccc7c8ccccc8n(c8cccc3c8c54)c67)c2)c1 ; c1ccc(N(c2ccc(-n3c4cccc5c6cccc7c8ccccc8n(c8cccc3c8c54)c67)cc2)c2ccc3c(c2)[se]c2ccccc23)cc1 ; c1ccc(N(c2cccc(-n3c4cccc5c6cccc7c8ccccc8n(c8cccc3c8c54)c67)c2)c2ccc3c(ccc4c5ccccc5oc34)c2)cc1]

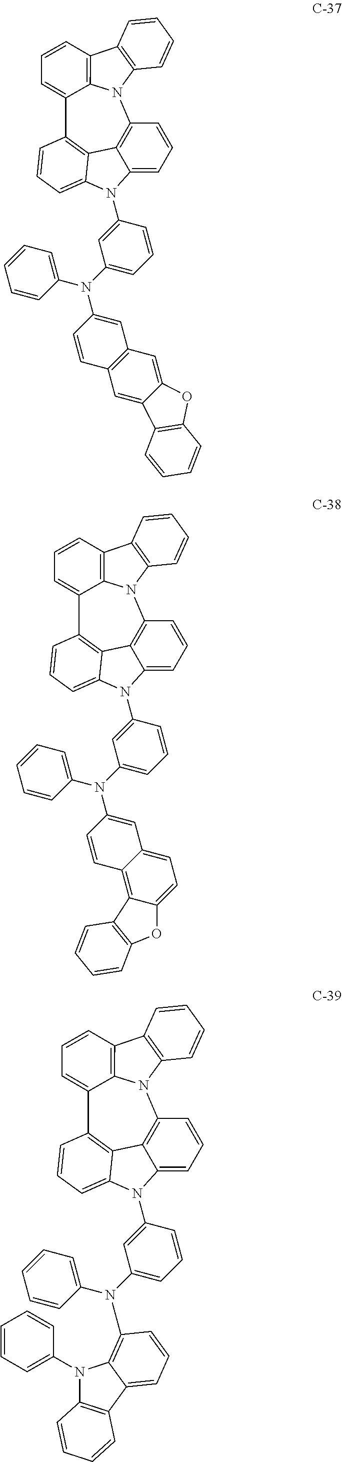

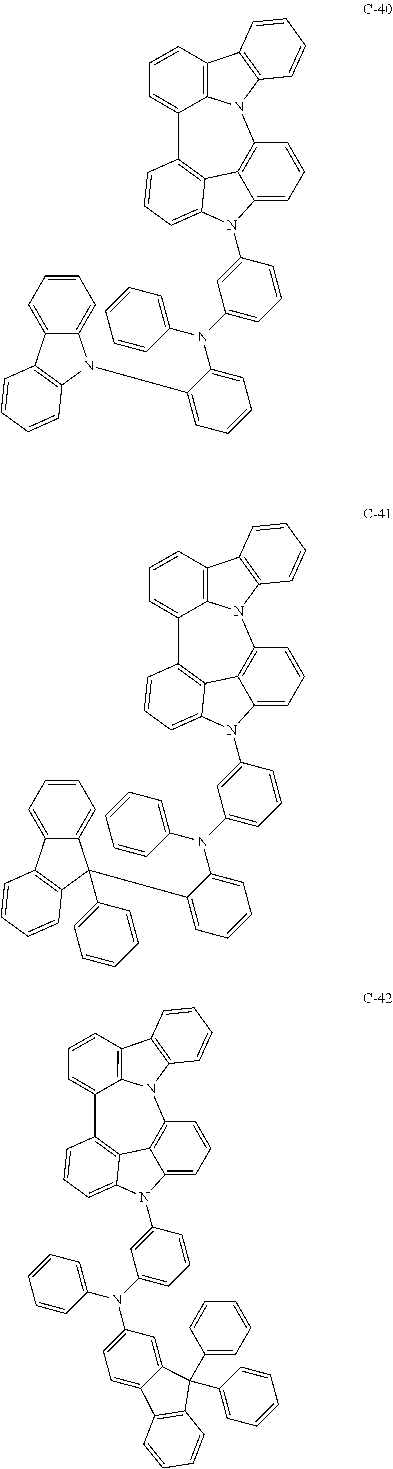 [SMILES: c1ccc(N(c2cccc(-n3c4cccc5c6cccc7c8ccccc8n(c8cccc3c8c54)c67)c2)c2ccccc2-n2c3ccccc3c3ccccc32)cc1 ; c1ccc(N(c2cccc(-n3c4cccc5c6cccc7c8ccccc8n(c8cccc3c8c54)c67)c2)c2ccccc2C2(c3ccccc3)c3ccccc3-c3ccccc32)cc1 ; c1ccc(N(c2cccc(-n3c4cccc5c6cccc7c8ccccc8n(c8cccc3c8c54)c67)c2)c2ccc3c(c2)C(c2ccccc2)(c2ccccc2)c2ccccc2-3)cc1]

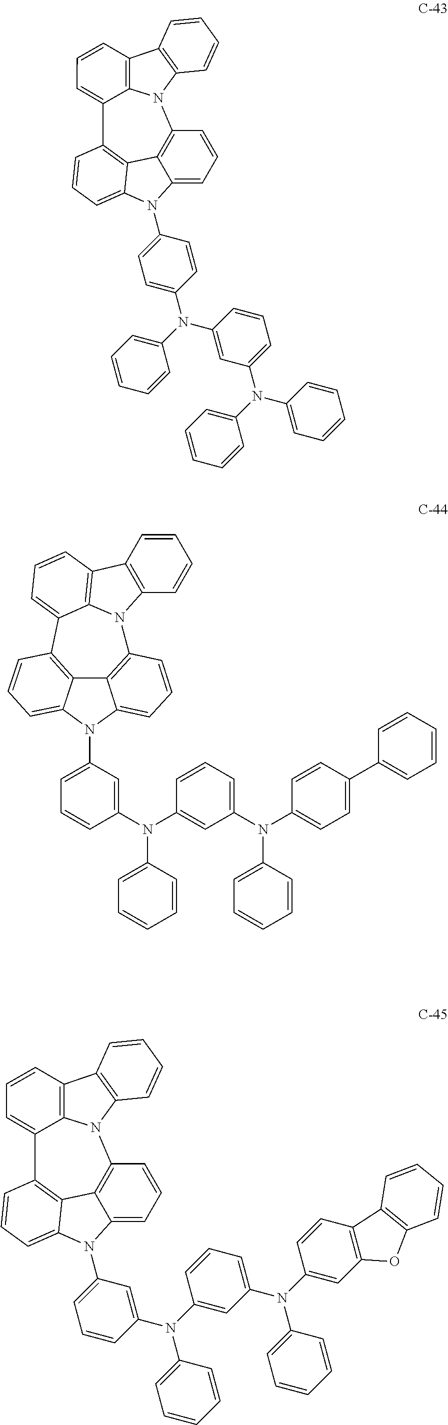 [SMILES: c1ccc(N(c2ccccc2)c2cccc(N(c3ccccc3)c3ccc(-n4c5cccc6c7cccc8c9ccccc9n(c9cccc4c9c65)c78)cc3)c2)cc1 ; c1ccc(-c2ccc(N(c3ccccc3)c3cccc(N(c4ccccc4)c4cccc(-n5c6cccc7c8cccc9c%10ccccc%10n(c%10cccc5c%10c76)c89)c4)c3)cc2)cc1 ; c1ccc(N(c2cccc(N(c3ccccc3)c3ccc4c(c3)oc3ccccc34)c2)c2cccc(-n3c4cccc5c6cccc7c8ccccc8n(c8cccc3c8c54)c67)c2)cc1]

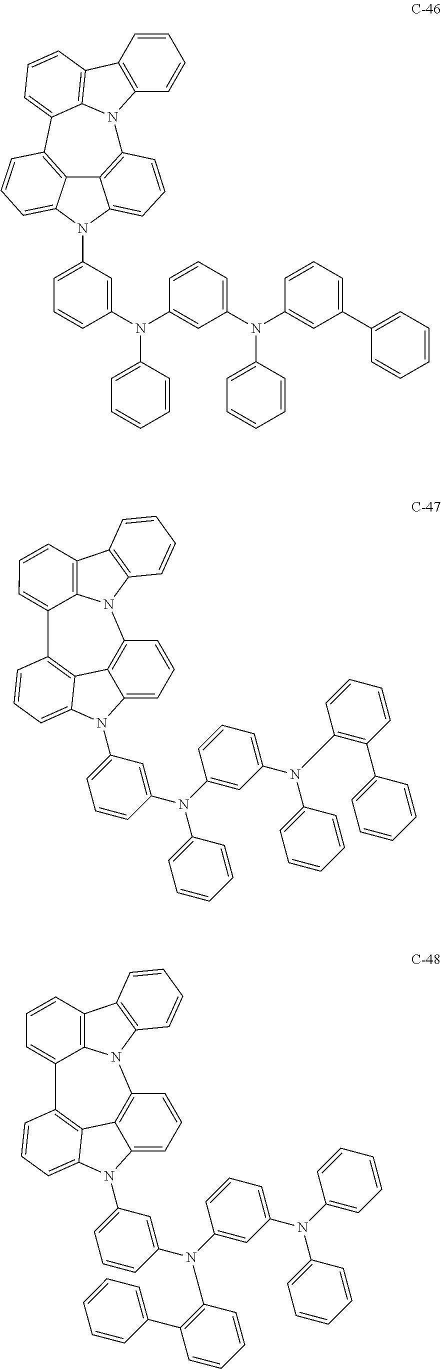 [SMILES: c1ccc(-c2cccc(N(c3ccccc3)c3cccc(N(c4ccccc4)c4cccc(-n5c6cccc7c8cccc9c%10ccccc%10n(c%10cccc5c%10c76)c89)c4)c3)c2)cc1 ; c1ccc(-c2ccccc2N(c2ccccc2)c2cccc(N(c3ccccc3)c3cccc(-n4c5cccc6c7cccc8c9ccccc9n(c9cccc4c9c65)c78)c3)c2)cc1 ; c1ccc(-c2ccccc2N(c2cccc(N(c3ccccc3)c3ccccc3)c2)c2cccc(-n3c4cccc5c6cccc7c8ccccc8n(c8cccc3c8c54)c67)c2)cc1]

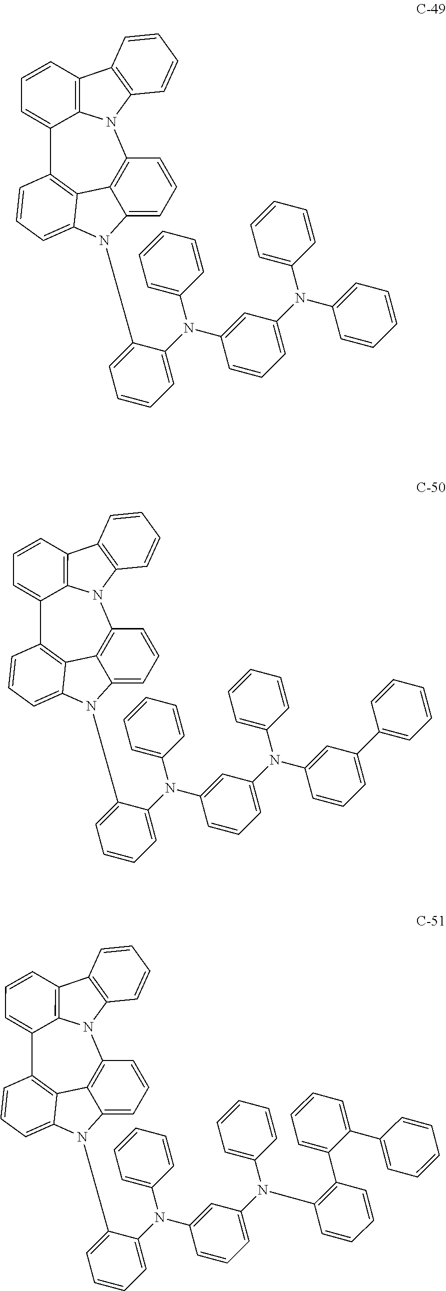 [SMILES: c1ccc(N(c2ccccc2)c2cccc(N(c3ccccc3)c3ccccc3-n3c4cccc5c6cccc7c8ccccc8n(c8cccc3c8c54)c67)c2)cc1 ; c1ccc(-c2cccc(N(c3ccccc3)c3cccc(N(c4ccccc4)c4ccccc4-n4c5cccc6c7cccc8c9ccccc9n(c9cccc4c9c65)c78)c3)c2)cc1 ; c1ccc(-c2ccccc2-c2ccccc2N(c2ccccc2)c2cccc(N(c3ccccc3)c3ccccc3-n3c4cccc5c6cccc7c8ccccc8n(c8cccc3c8c54)c67)c2)cc1]

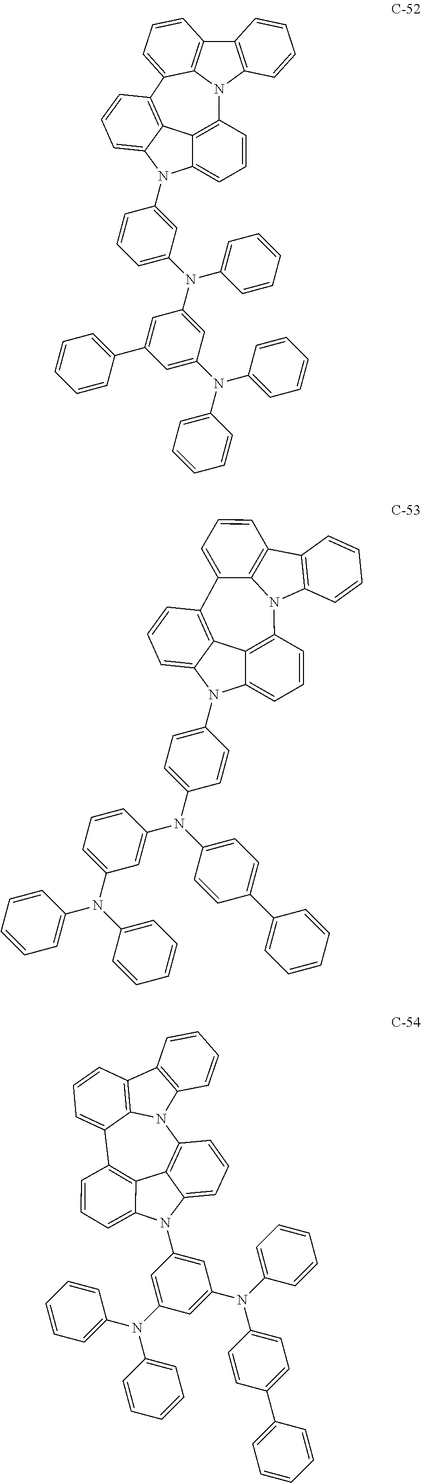 [SMILES: c1ccc(-c2cc(N(c3ccccc3)c3ccccc3)cc(N(c3ccccc3)c3cccc(-n4c5cccc6c7cccc8c9ccccc9n(c9cccc4c9c65)c78)c3)c2)cc1 ; c1ccc(-c2ccc(N(c3ccc(-n4c5cccc6c7cccc8c9ccccc9n(c9cccc4c9c65)c78)cc3)c3cccc(N(c4ccccc4)c4ccccc4)c3)cc2)cc1 ; c1ccc(-c2ccc(N(c3ccccc3)c3cc(N(c4ccccc4)c4ccccc4)cc(-n4c5cccc6c7cccc8c9ccccc9n(c9cccc4c9c65)c78)c3)cc2)cc1]

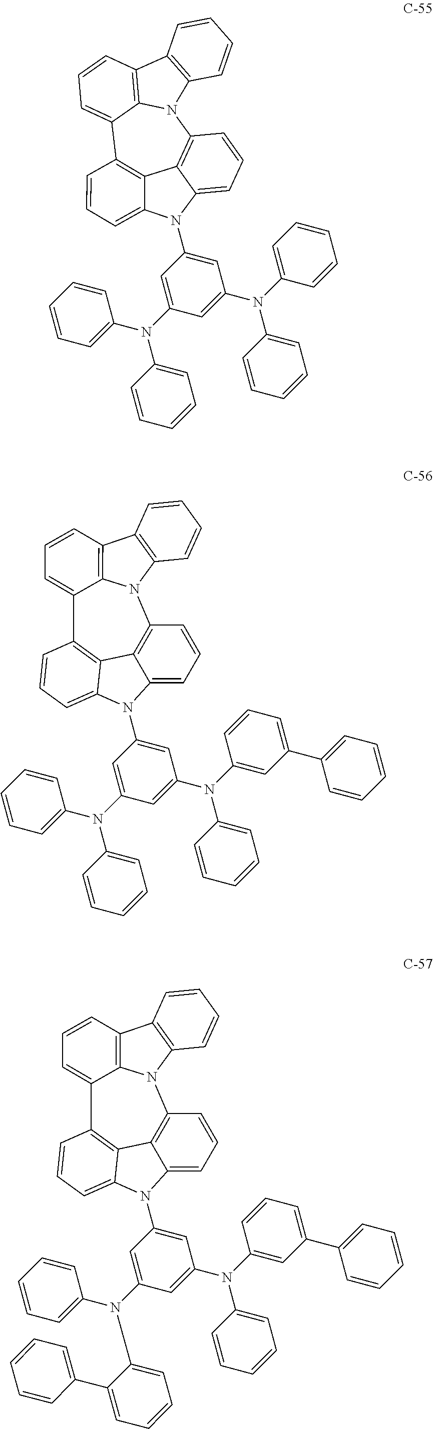 [SMILES: c1ccc(N(c2ccccc2)c2cc(N(c3ccccc3)c3ccccc3)cc(-n3c4cccc5c6cccc7c8ccccc8n(c8cccc3c8c54)c67)c2)cc1 ; c1ccc(-c2cccc(N(c3ccccc3)c3cc(N(c4ccccc4)c4ccccc4)cc(-n4c5cccc6c7cccc8c9ccccc9n(c9cccc4c9c65)c78)c3)c2)cc1 ; c1ccc(-c2cccc(N(c3ccccc3)c3cc(N(c4ccccc4)c4ccccc4-c4ccccc4)cc(-n4c5cccc6c7cccc8c9ccccc9n(c9cccc4c9c65)c78)c3)c2)cc1]

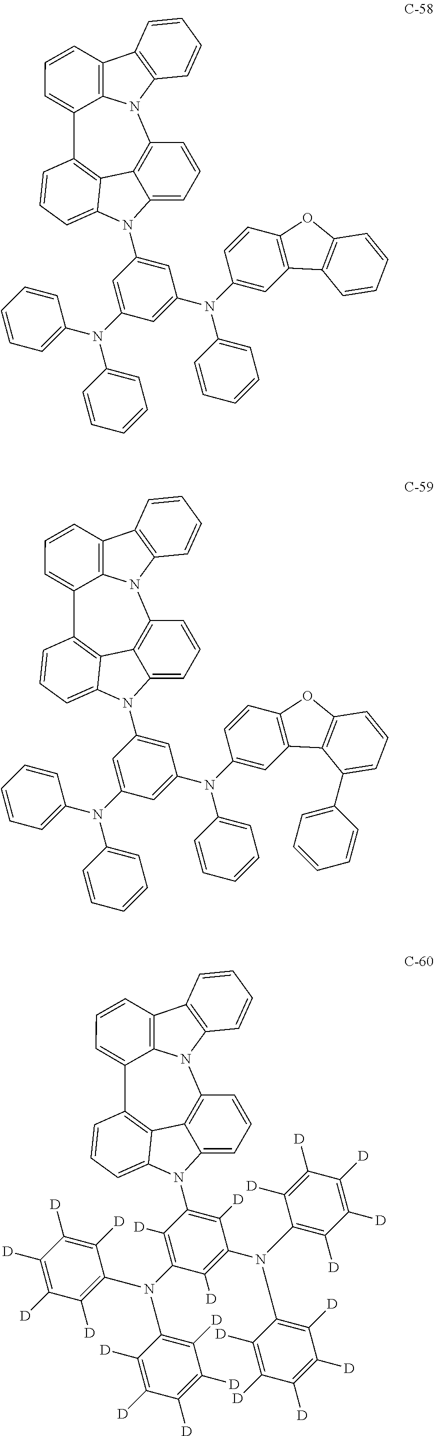 [SMILES: c1ccc(N(c2ccccc2)c2cc(N(c3ccccc3)c3ccc4oc5ccccc5c4c3)cc(-n3c4cccc5c6cccc7c8ccccc8n(c8cccc3c8c54)c67)c2)cc1 ; c1ccc(-c2cccc3oc4ccc(N(c5ccccc5)c5cc(N(c6ccccc6)c6ccccc6)cc(-n6c7cccc8c9cccc%10c%11ccccc%11n(c%11cccc6c%11c87)c9%10)c5)cc4c23)cc1 ; [2H]c1c([2H])c([2H])c(N(c2c([2H])c([2H])c([2H])c([2H])c2[2H])c2c([2H])c(N(c3c([2H])c([2H])c([2H])c([2H])c3[2H])c3c([2H])c([2H])c([2H])c([2H])c3[2H])c([2H])c(-n3c4cccc5c6cccc7c8ccccc8n(c8cccc3c8c54)c67)c2[2H])c([2H])c1[2H]]

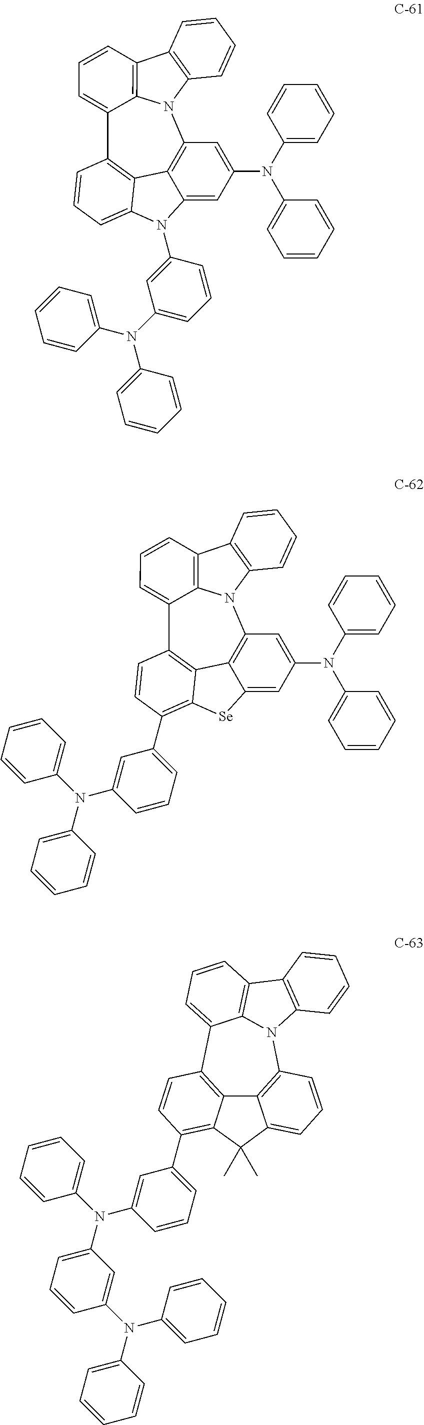 [SMILES: c1ccc(N(c2ccccc2)c2cccc(-n3c4cccc5c6cccc7c8ccccc8n(c8cc(N(c9ccccc9)c9ccccc9)cc3c8c54)c67)c2)cc1 ; c1ccc(N(c2ccccc2)c2cccc(-c3ccc4c5cccc6c7ccccc7n(c7cc(N(c8ccccc8)c8ccccc8)cc8[se]c3c4c87)c56)c2)cc1 ; CC1(C)c2cccc3c2-c2c(ccc(-c4cccc(N(c5ccccc5)c5cccc(N(c6ccccc6)c6ccccc6)c5)c4)c21)-c1cccc2c4ccccc4n-3c12]

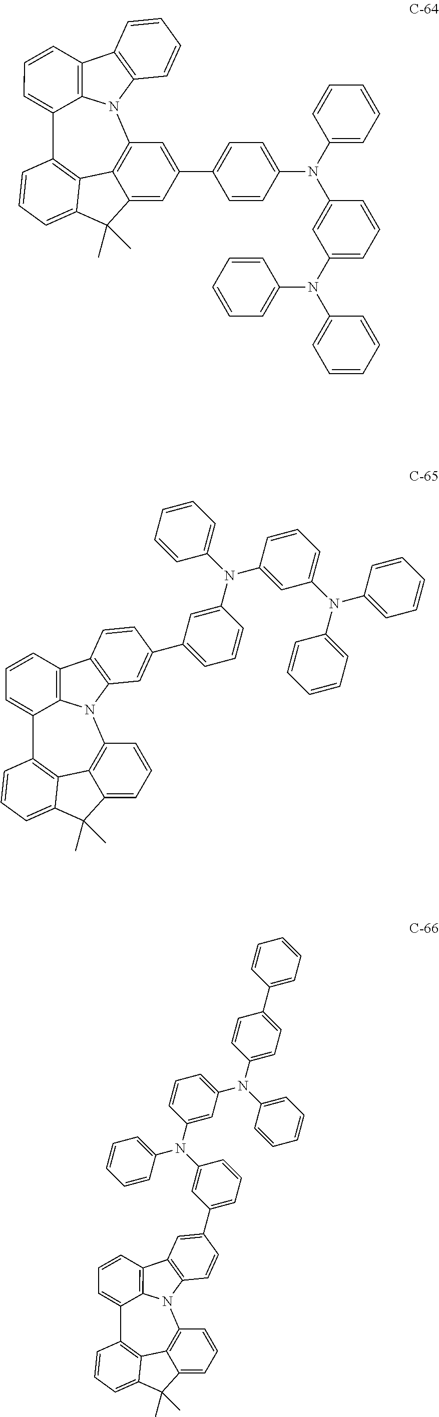 [SMILES: CC1(C)c2cccc3c2-c2c(cc(-c4ccc(N(c5ccccc5)c5cccc(N(c6ccccc6)c6ccccc6)c5)cc4)cc21)-n1c2ccccc2c2cccc-3c21 ; CC1(C)c2cccc3c2-c2c(cccc21)-n1c2cc(-c4cccc(N(c5ccccc5)c5cccc(N(c6ccccc6)c6ccccc6)c5)c4)ccc2c2cccc-3c21 ; CC1(C)c2cccc3c2-c2c(cccc21)-n1c2ccc(-c4cccc(N(c5ccccc5)c5cccc(N(c6ccccc6)c6ccc(-c7ccccc7)cc6)c5)c4)cc2c2cccc-3c21]

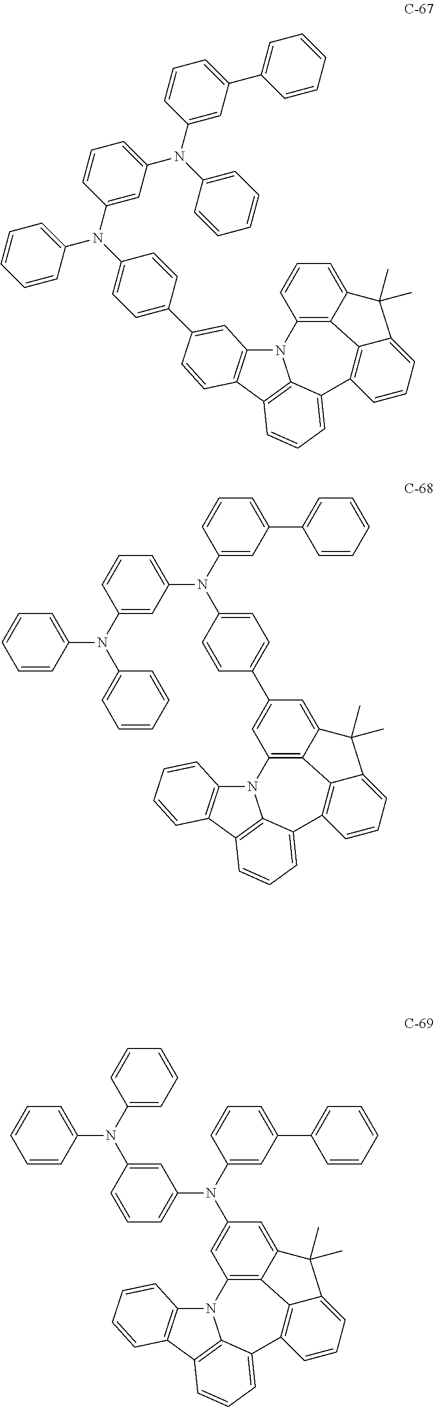 [SMILES: CC1(C)c2cccc3c2-c2c(cccc21)-n1c2cc(-c4ccc(N(c5ccccc5)c5cccc(N(c6ccccc6)c6cccc(-c7ccccc7)c6)c5)cc4)ccc2c2cccc-3c21 ; CC1(C)c2cccc3c2-c2c(cc(-c4ccc(N(c5cccc(-c6ccccc6)c5)c5cccc(N(c6ccccc6)c6ccccc6)c5)cc4)cc21)-n1c2ccccc2c2cccc-3c21 ; CC1(C)c2cccc3c2-c2c(cc(N(c4cccc(-c5ccccc5)c4)c4cccc(N(c5ccccc5)c5ccccc5)c4)cc21)-n1c2ccccc2c2cccc-3c21]

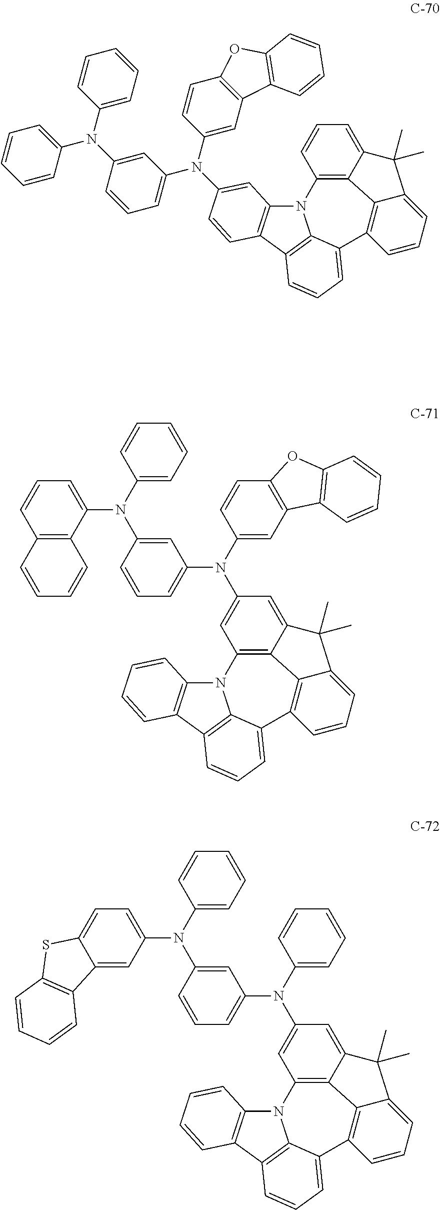 [SMILES: CC1(C)c2cccc3c2-c2c(cccc21)-n1c2cc(N(c4cccc(N(c5ccccc5)c5ccccc5)c4)c4ccc5oc6ccccc6c5c4)ccc2c2cccc-3c21 ; CC1(C)c2cccc3c2-c2c(cc(N(c4cccc(N(c5ccccc5)c5cccc6ccccc56)c4)c4ccc5oc6ccccc6c5c4)cc21)-n1c2ccccc2c2cccc-3c21 ; CC1(C)c2cccc3c2-c2c(cc(N(c4ccccc4)c4cccc(N(c5ccccc5)c5ccc6sc7ccccc7c6c5)c4)cc21)-n1c2ccccc2c2cccc-3c21]

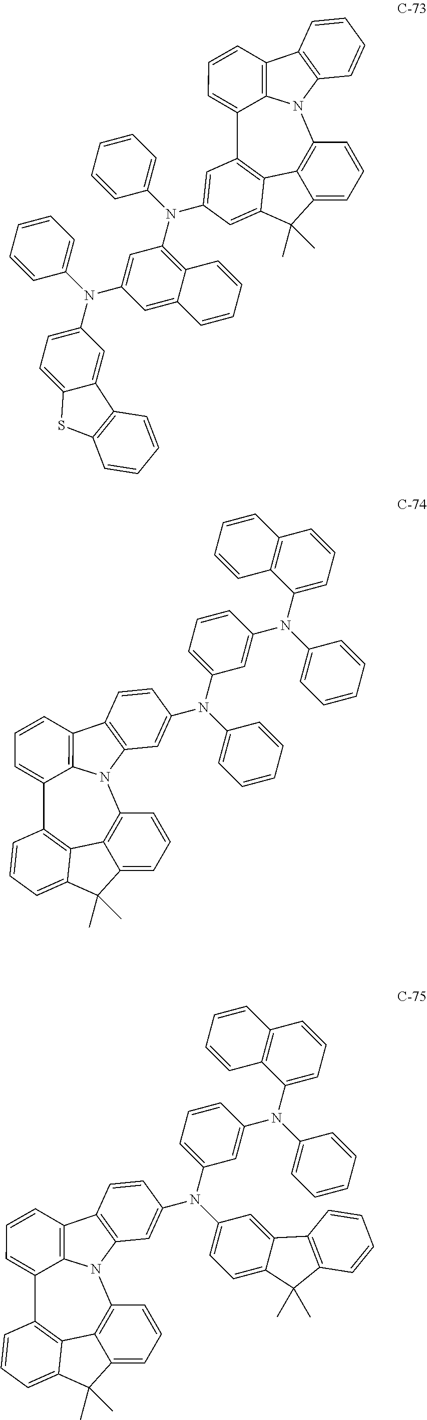 [SMILES: CC1(C)c2cc(N(c3ccccc3)c3cc(N(c4ccccc4)c4ccc5sc6ccccc6c5c4)cc4ccccc34)cc3c2-c2c(cccc21)-n1c2ccccc2c2cccc-3c21 ; CC1(C)c2cccc3c2-c2c(cccc21)-n1c2cc(N(c4ccccc4)c4cccc(N(c5ccccc5)c5cccc6ccccc56)c4)ccc2c2cccc-3c21 ; CC1(C)c2ccccc2-c2cc(N(c3cccc(N(c4ccccc4)c4cccc5ccccc45)c3)c3ccc4c5cccc6c5n(c4c3)-c3cccc4c3-c3c-6cccc3C4(C)C)ccc21]

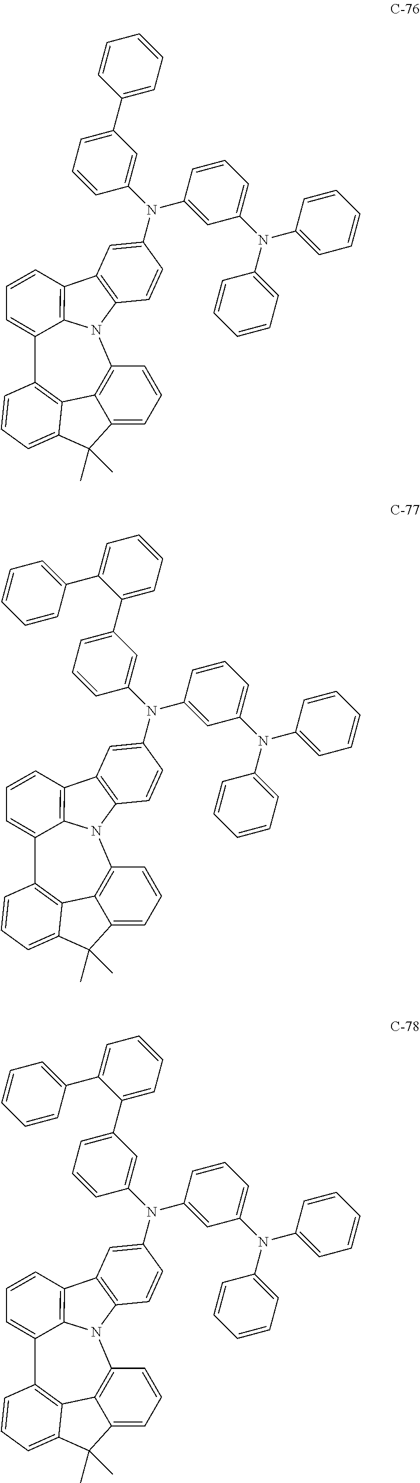 [SMILES: CC1(C)c2cccc3c2-c2c(cccc21)-n1c2ccc(N(c4cccc(-c5ccccc5)c4)c4cccc(N(c5ccccc5)c5ccccc5)c4)cc2c2cccc-3c21 ; CC1(C)c2cccc3c2-c2c(cccc21)-n1c2ccc(N(c4cccc(-c5ccccc5-c5ccccc5)c4)c4cccc(N(c5ccccc5)c5ccccc5)c4)cc2c2cccc-3c21 ; CC1(C)c2cccc3c2-c2c(cccc21)-n1c2ccc(N(c4cccc(-c5ccccc5-c5ccccc5)c4)c4cccc(N(c5ccccc5)c5ccccc5)c4)cc2c2cccc-3c21]

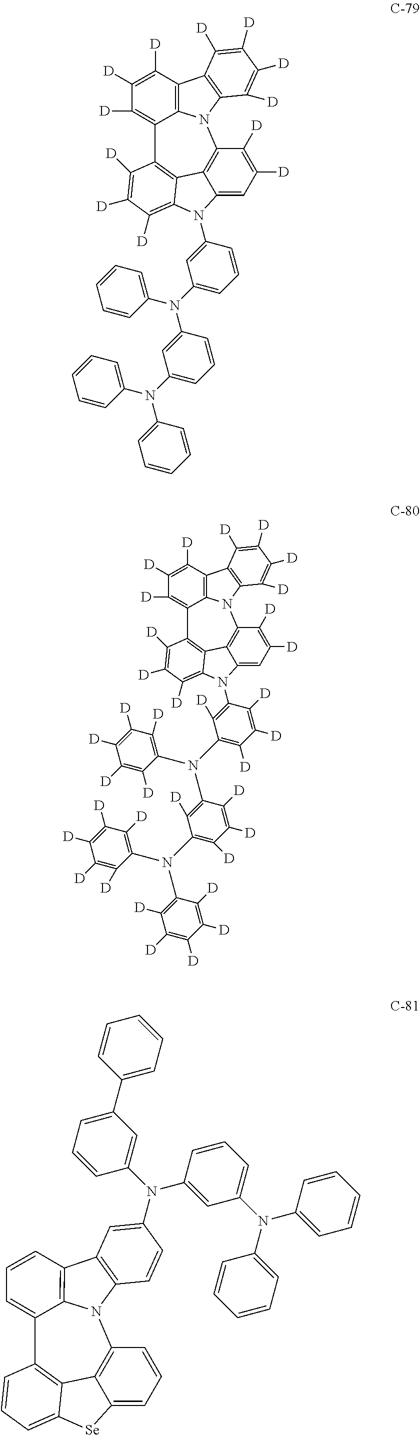

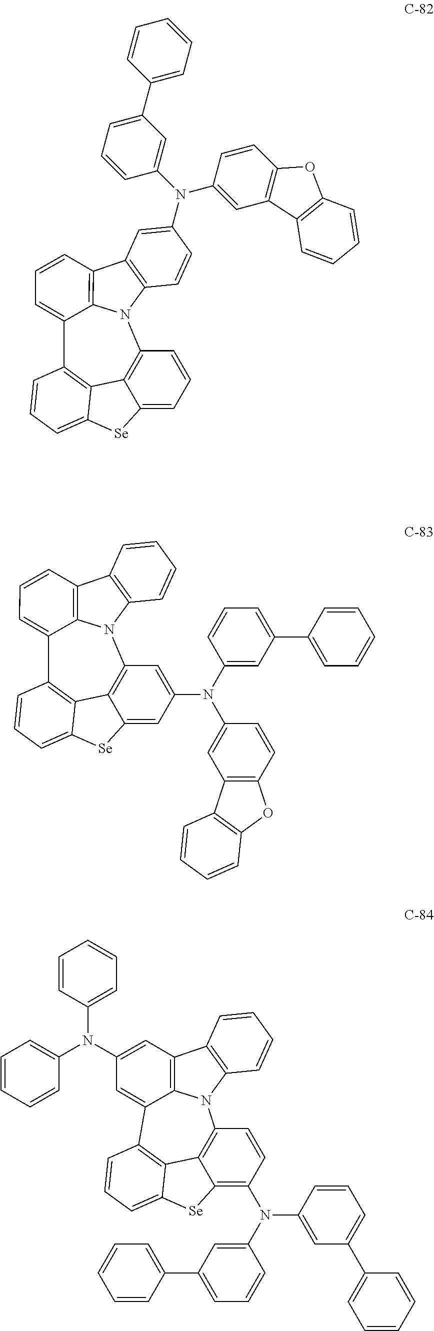 [SMILES: c1ccc(-c2cccc(N(c3ccc4oc5ccccc5c4c3)c3ccc4c(c3)c3cccc5c6cccc7[se]c8cccc(c8c76)n4c53)c2)cc1 ; c1ccc(-c2cccc(N(c3ccc4oc5ccccc5c4c3)c3cc4[se]c5cccc6c7cccc8c9ccccc9n(c(c3)c4c56)c78)c2)cc1 ; c1ccc(-c2cccc(N(c3cccc(-c4ccccc4)c3)c3ccc4c5c3[se]c3cccc(c6cc(N(c7ccccc7)c7ccccc7)cc7c8ccccc8n4c67)c35)c2)cc1]

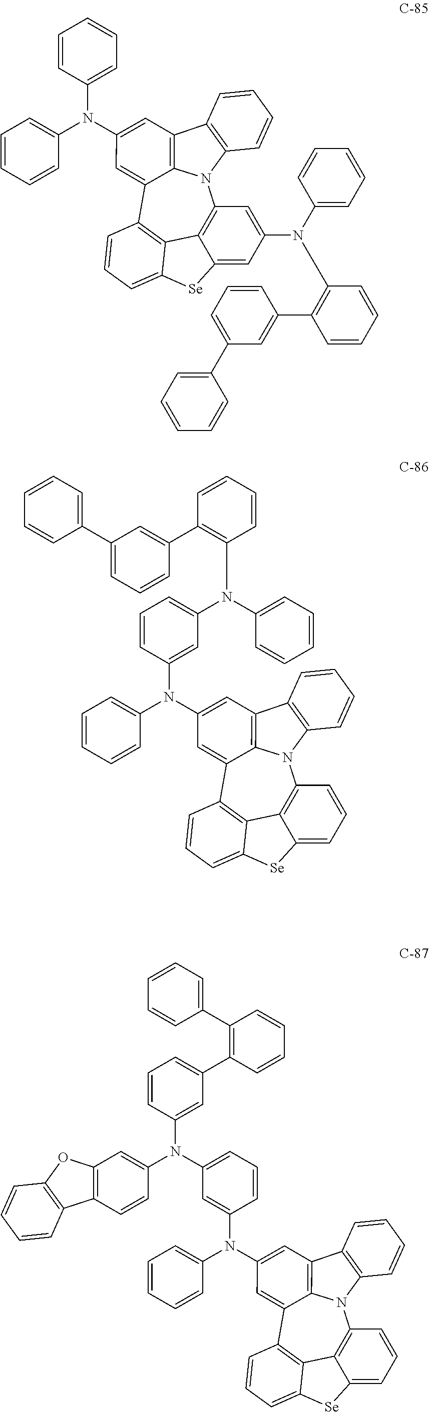 [SMILES: c1ccc(-c2cccc(-c3ccccc3N(c3ccccc3)c3cc4[se]c5cccc6c7cc(N(c8ccccc8)c8ccccc8)cc8c9ccccc9n(c(c3)c4c56)c78)c2)cc1 ; c1ccc(-c2cccc(-c3ccccc3N(c3ccccc3)c3cccc(N(c4ccccc4)c4cc5c6cccc7[se]c8cccc(c8c76)n6c7ccccc7c(c4)c56)c3)c2)cc1 ; c1ccc(-c2ccccc2-c2cccc(N(c3cccc(N(c4ccccc4)c4cc5c6cccc7[se]c8cccc(c8c76)n6c7ccccc7c(c4)c56)c3)c3ccc4c(c3)oc3ccccc34)c2)cc1]

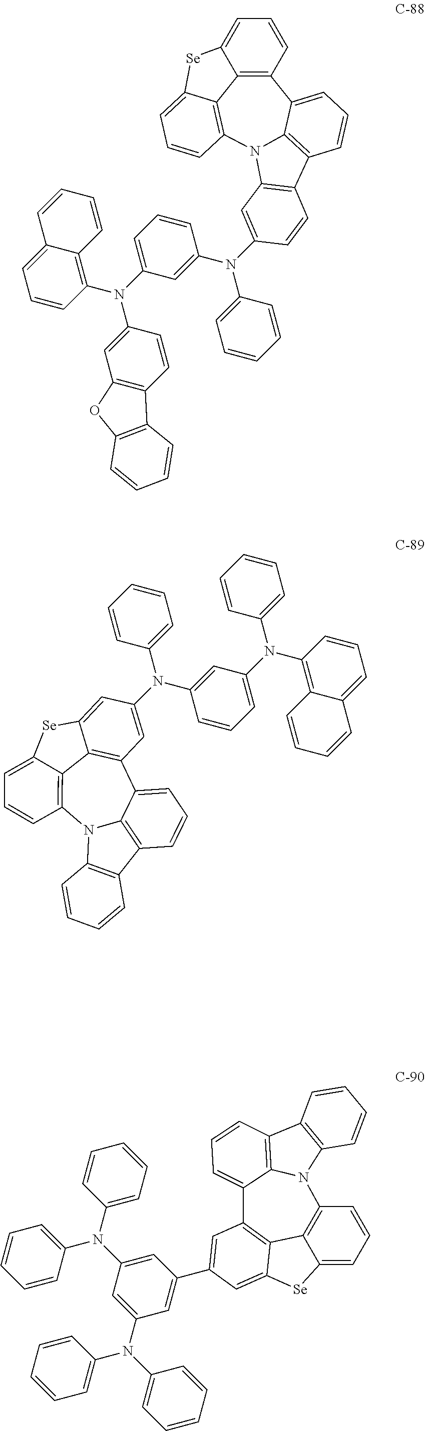 [SMILES: c1ccc(N(c2cccc(N(c3ccc4c(c3)oc3ccccc34)c3cccc4ccccc34)c2)c2ccc3c4cccc5c6cccc7[se]c8cccc(c8c76)n(c3c2)c54)cc1 ; c1ccc(N(c2cccc(N(c3ccccc3)c3cccc4ccccc34)c2)c2cc3[se]c4cccc5c4c3c(c2)c2cccc3c4ccccc4n5c23)cc1 ; c1ccc(N(c2ccccc2)c2cc(-c3cc4[se]c5cccc6c5c4c(c3)c3cccc4c5ccccc5n6c34)cc(N(c3ccccc3)c3ccccc3)c2)cc1]

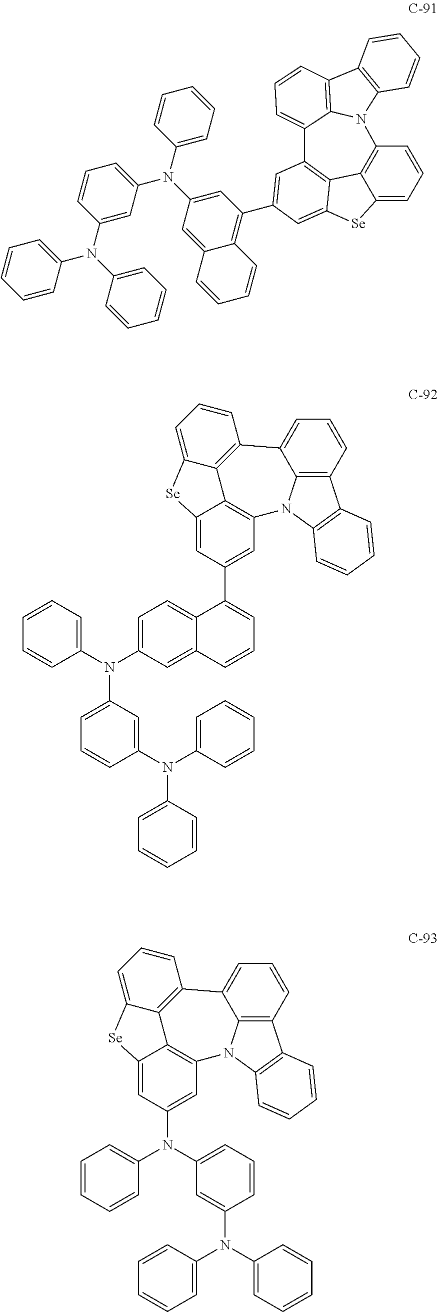 [SMILES: c1ccc(N(c2ccccc2)c2cccc(N(c3ccccc3)c3cc(-c4cc5[se]c6cccc7c6c5c(c4)c4cccc5c6ccccc6n7c45)c4ccccc4c3)c2)cc1 ; c1ccc(N(c2ccccc2)c2cccc(N(c3ccccc3)c3ccc4c(-c5cc6[se]c7cccc8c9cccc%10c%11ccccc%11n(c(c5)c6c78)c9%10)cccc4c3)c2)cc1 ; c1ccc(N(c2ccccc2)c2cccc(N(c3ccccc3)c3cc4[se]c5cccc6c7cccc8c9ccccc9n(c(c3)c4c56)c78)c2)cc1]

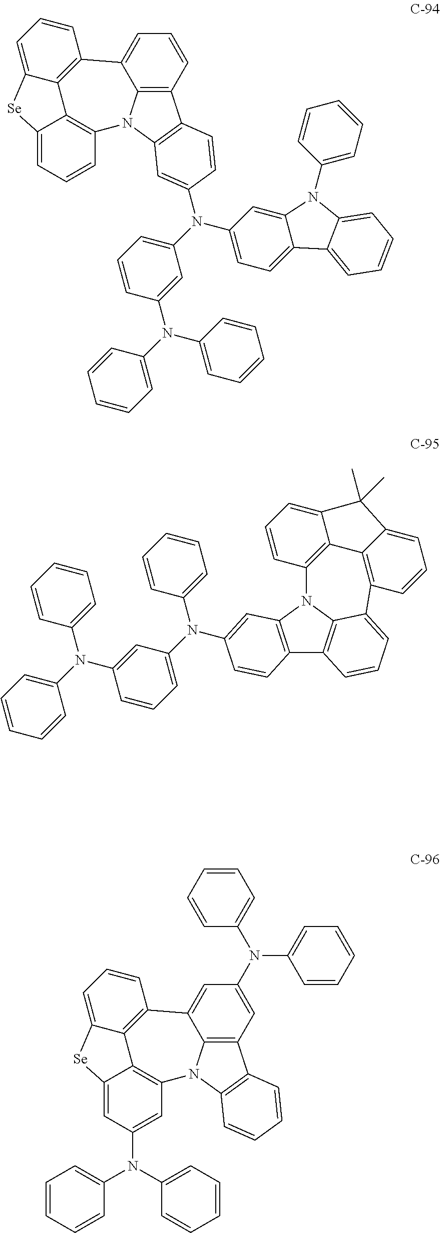 [SMILES: c1ccc(N(c2ccccc2)c2cccc(N(c3ccc4c5ccccc5n(-c5ccccc5)c4c3)c3ccc4c5cccc6c7cccc8[se]c9cccc(c9c87)n(c4c3)c65)c2)cc1 ; CC1(C)c2cccc3c2-c2c(cccc21)-n1c2cc(N(c4ccccc4)c4cccc(N(c5ccccc5)c5ccccc5)c4)ccc2c2cccc-3c21 ; c1ccc(N(c2ccccc2)c2cc3c4cccc5[se]c6cc(N(c7ccccc7)c7ccccc7)cc(c6c54)n4c5ccccc5c(c2)c34)cc1]

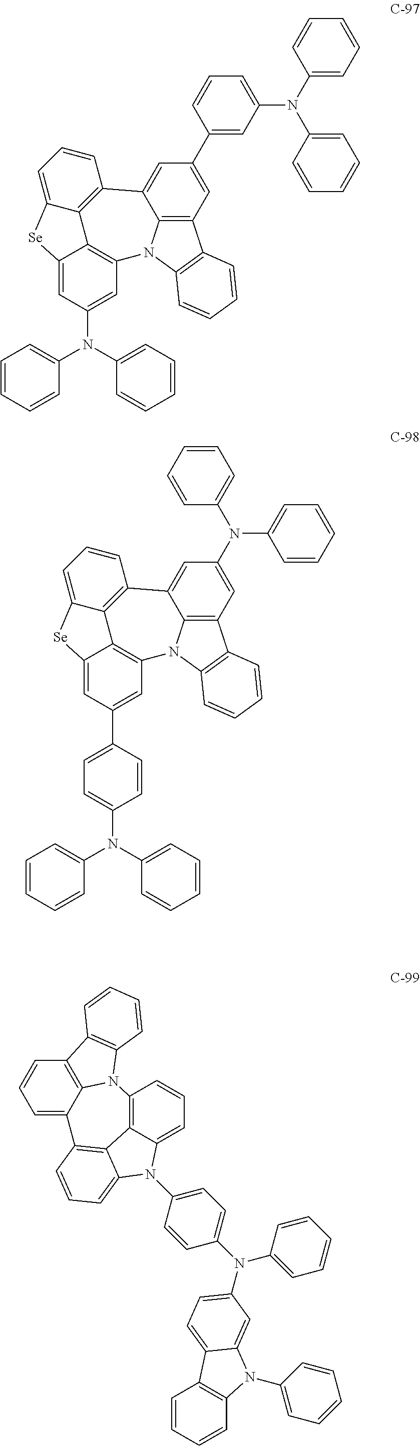 [SMILES: c1ccc(N(c2ccccc2)c2cccc(-c3cc4c5cccc6[se]c7cc(N(c8ccccc8)c8ccccc8)cc(c7c65)n5c6ccccc6c(c3)c45)c2)cc1 ; c1ccc(N(c2ccccc2)c2ccc(-c3cc4[se]c5cccc6c7cc(N(c8ccccc8)c8ccccc8)cc8c9ccccc9n(c(c3)c4c56)c78)cc2)cc1 ; c1ccc(N(c2ccc(-n3c4cccc5c6cccc7c8ccccc8n(c8cccc3c8c54)c67)cc2)c2ccc3c4ccccc4n(-c4ccccc4)c3c2)cc1]

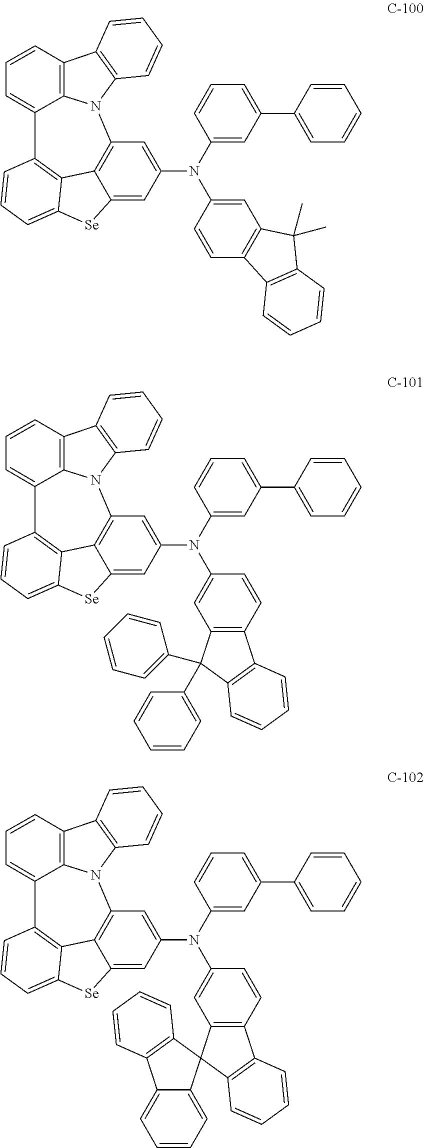

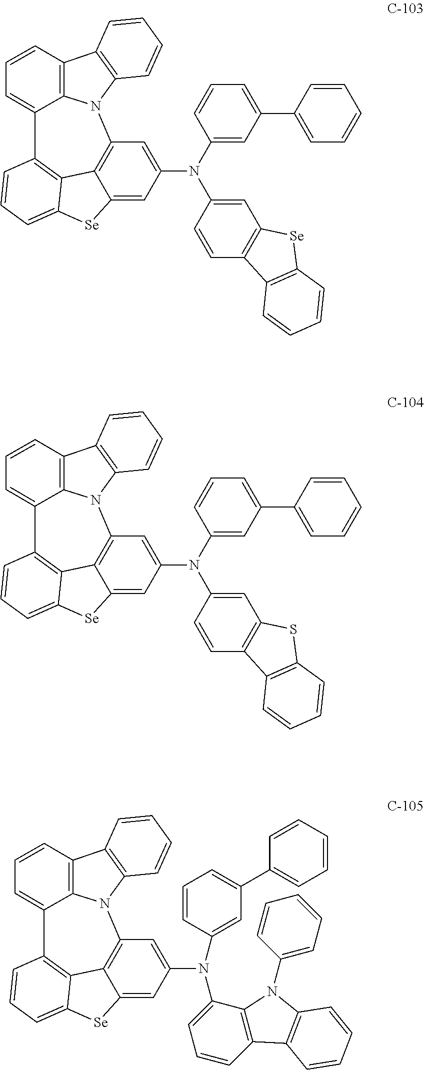 [SMILES: c1ccc(-c2cccc(N(c3ccc4c(c3)[se]c3ccccc34)c3cc4[se]c5cccc6c7cccc8c9ccccc9n(c(c3)c4c56)c78)c2)cc1 ; c1ccc(-c2cccc(N(c3ccc4c(c3)sc3ccccc34)c3cc4[se]c5cccc6c7cccc8c9ccccc9n(c(c3)c4c56)c78)c2)cc1 ; c1ccc(-c2cccc(N(c3cc4[se]c5cccc6c7cccc8c9ccccc9n(c(c3)c4c56)c78)c3cccc4c5ccccc5n(-c5ccccc5)c34)c2)cc1]

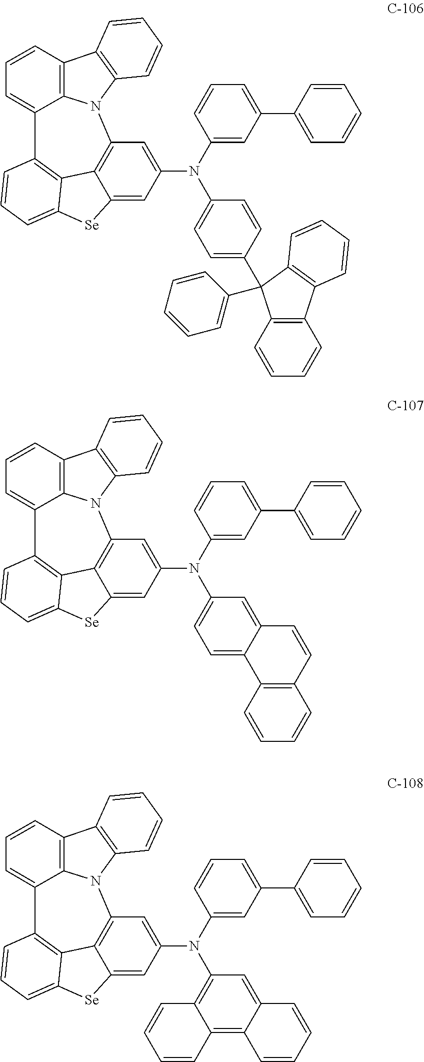 [SMILES: c1ccc(-c2cccc(N(c3ccc(C4(c5ccccc5)c5ccccc5-c5ccccc54)cc3)c3cc4[se]c5cccc6c7cccc8c9ccccc9n(c(c3)c4c56)c78)c2)cc1 ; c1ccc(-c2cccc(N(c3ccc4c(ccc5ccccc54)c3)c3cc4[se]c5cccc6c7cccc8c9ccccc9n(c(c3)c4c56)c78)c2)cc1 ; c1ccc(-c2cccc(N(c3cc4[se]c5cccc6c7cccc8c9ccccc9n(c(c3)c4c56)c78)c3cc4ccccc4c4ccccc34)c2)cc1]

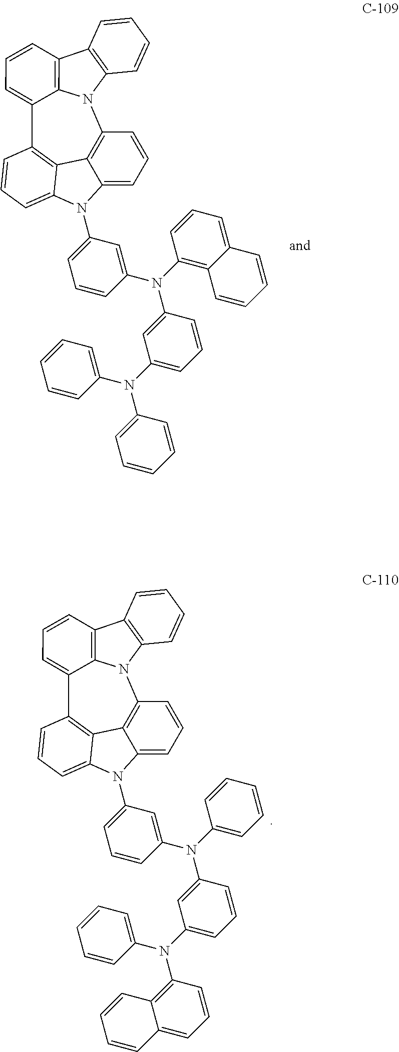 [SMILES: c1ccc(N(c2ccccc2)c2cccc(N(c3cccc(-n4c5cccc6c7cccc8c9ccccc9n(c9cccc4c9c65)c78)c3)c3cccc4ccccc34)c2)cc1 ; c1ccc(N(c2cccc(N(c3ccccc3)c3cccc4ccccc34)c2)c2cccc(-n3c4cccc5c6cccc7c8ccccc8n(c8cccc3c8c54)c67)c2)cc1]

10. An organic electroluminescent device comprising the organic electroluminescent compound according to claim 8.

11. An organic electroluminescent compound represented by any one of the following formulas 2′-1 to 2′-4: 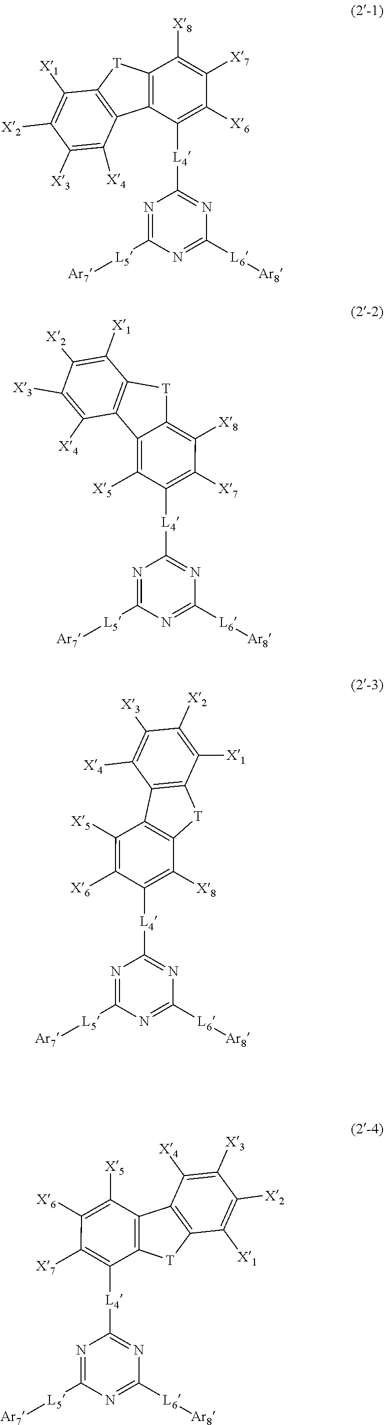
in formulas 2-1 to 2-4,
T represents O or S;
X₁′ to X₄′, each independently, represent hydrogen, deuterium, a substituted or unsubstituted (C1-C30)alkyl, a substituted or unsubstituted (C6-C30)aryl, or a substituted or unsubstituted (3- to 30-membered)heteroaryl;
L₄′ to L₆′, each independently, represent a single bond, a phenylene unsubstituted or substituted with deuterium, or a naphthylene unsubstituted or substituted with deuterium; and
Ar₇′ and Ar₈′, each independently, represent a phenyl unsubstituted or substituted with deuterium, a biphenyl unsubstituted or substituted with deuterium, a naphthyl unsubstituted or substituted with deuterium, a substituted or unsubstituted pyridyl, a substituted or unsubstituted dibenzofuranyl, a substituted or unsubstituted dibenzothiophenyl, a substituted or unsubstituted benzonaphthofuranyl, or a substituted or unsubstituted benzonaphthothiophenyl;
in formula 2′-1,
X₆′ to X₈′, each independently, represent hydrogen, deuterium or -L₇-Ar₉, with the proviso that at least one of X₆′ to X₃′ represents -L₇-Ar₉;
if X₆′ is -L₇-Ar₉, L₇ represents a single bond, a phenylene unsubstituted or substituted with deuterium, or a naphthylene unsubstituted or substituted with deuterium, and Ar₉ represents a phenyl unsubstituted or substituted with deuterium, a biphenyl unsubstituted or substituted with deuterium, or a naphthyl unsubstituted or substituted with deuterium; with the proviso that if L₇ is a single bond, Ar₉ represents a naphthyl unsubstituted or substituted with deuterium;
if X₇′ is -L₇-Ar₉, L₇ represents a single bond, a phenylene unsubstituted or substituted with deuterium, or a naphthylene unsubstituted or substituted with deuterium, and Ar₉ represents a phenyl unsubstituted or substituted with deuterium, a biphenyl unsubstituted or substituted with deuterium, or a naphthyl unsubstituted or substituted with deuterium;
if X₈′ is -L₇-Ar₉, L₇ represents a single bond, a phenylene unsubstituted or substituted with deuterium, or a naphthylene unsubstituted or substituted with deuterium, and Ar₉ represents a phenyl unsubstituted or substituted with deuterium, or a naphthyl unsubstituted or substituted with deuterium; and
with the proviso that if X₈′ is -L₇-Ar₉ and a phenyl, at least one of Ar₇′ and Ar₈′ represents a dibenzofuranyl or a dibenzothiophenyl;
in formula 2′-2,
X₈′ represents -L₇-Ar₉, and X₆′ and X₇′, each independently, represent hydrogen or deuterium;
L₇ represents a single bond, a phenylene unsubstituted or substituted with deuterium, or a naphthylene unsubstituted or substituted with deuterium, and Ar₉ represents hydrogen, a phenyl unsubstituted or substituted with deuterium, a biphenyl unsubstituted or substituted with deuterium, or a naphthyl unsubstituted or substituted with deuterium or a phenyl(s); and
with the proviso that if L₇ is a single bond, Ar₉ represents a naphthyl unsubstituted or substituted with deuterium;
in formula 2′-3,
X₅′, X₆′ and X₈′, each independently, represent hydrogen, deuterium, or -L₇-Ar₉, with the proviso that at least one of X₅′, X₆′ and X₈′ represents -L₇-Ar₉;
L₇ represents a single bond, a phenylene unsubstituted or substituted with deuterium, or a naphthylene unsubstituted or substituted with deuterium, and Ar₉ represents a phenyl unsubstituted or substituted with deuterium or a naphthyl(s), a biphenyl unsubstituted or substituted with deuterium, or a naphthyl unsubstituted or substituted with deuterium; and
with the proviso that if X₅′ is -L₇-Ar₉ and a naphthyl, at least one of Ar₇′ and Ar₈′ represents a substituted or unsubstituted naphthyl, a substituted or unsubstituted dibenzothiophenyl, or a substituted or unsubstituted dibenzofuranyl;
in formula 2′-4,
X₅′ to X₇, each independently, represent hydrogen, deuterium, or -L₇-Ar₉, with the proviso that at least one of X₅′ to X₇′ represents -L₇-Ar₉; and
L₇ represents a single bond, a phenylene unsubstituted or substituted with deuterium, or a naphthylene unsubstituted or substituted with deuterium, and Ar₉ represents a phenyl unsubstituted or substituted with deuterium, a biphenyl unsubstituted or substituted with deuterium, or a naphthyl unsubstituted or substituted with deuterium.

12. The organic electroluminescent compound according to claim 11, wherein the organic electroluminescent compound represented by any one of formulas 2′-1 to 2′-4 is selected from the group consisting of the following compounds:

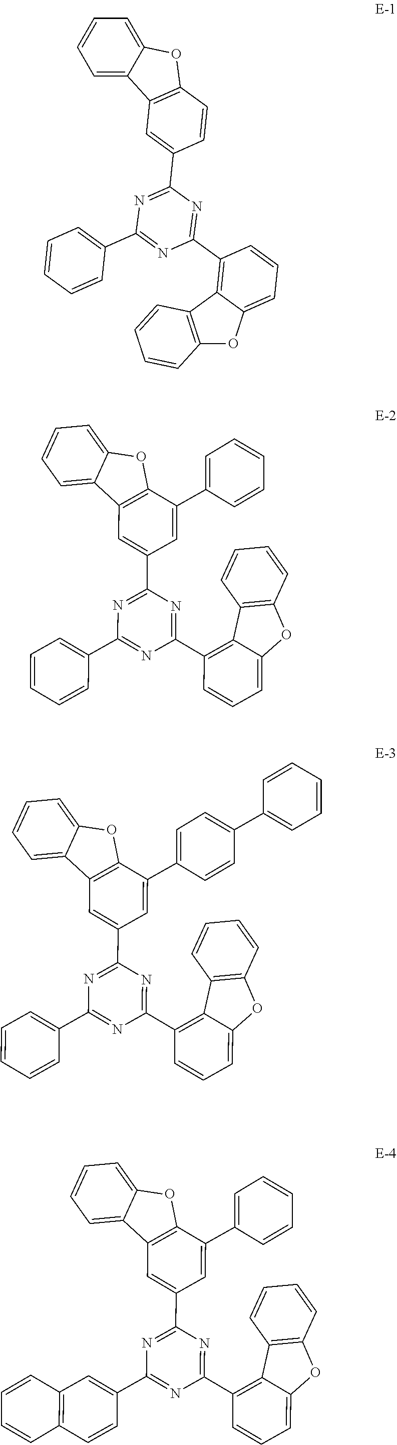 [SMILES: c1ccc(-c2nc(-c3ccc4oc5ccccc5c4c3)nc(-c3cccc4oc5ccccc5c34)n2)cc1 ; c1ccc(-c2nc(-c3cc(-c4ccccc4)c4oc5ccccc5c4c3)nc(-c3cccc4oc5ccccc5c34)n2)cc1 ; c1ccc(-c2ccc(-c3cc(-c4nc(-c5ccccc5)nc(-c5cccc6oc7ccccc7c56)n4)cc4c3oc3ccccc34)cc2)cc1 ; c1ccc(-c2cc(-c3nc(-c4ccc5ccccc5c4)nc(-c4cccc5oc6ccccc6c45)n3)cc3c2oc2ccccc23)cc1]

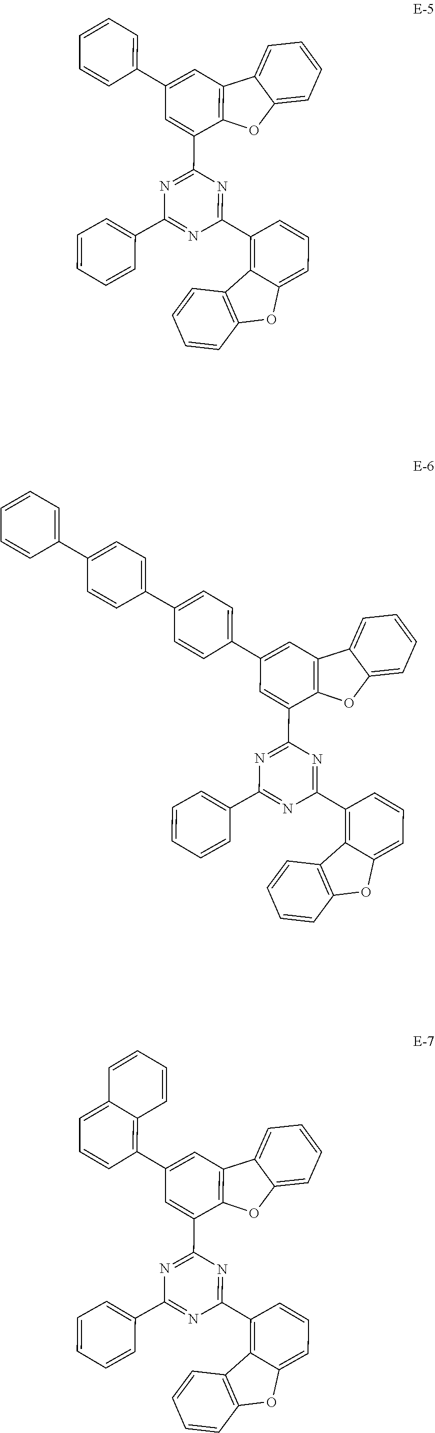 [SMILES: c1ccc(-c2cc(-c3nc(-c4ccccc4)nc(-c4cccc5oc6ccccc6c45)n3)c3oc4ccccc4c3c2)cc1 ; c1ccc(-c2ccc(-c3ccc(-c4cc(-c5nc(-c6ccccc6)nc(-c6cccc7oc8ccccc8c67)n5)c5oc6ccccc6c5c4)cc3)cc2)cc1 ; c1ccc(-c2nc(-c3cc(-c4cccc5ccccc45)cc4c3oc3ccccc34)nc(-c3cccc4oc5ccccc5c34)n2)cc1]

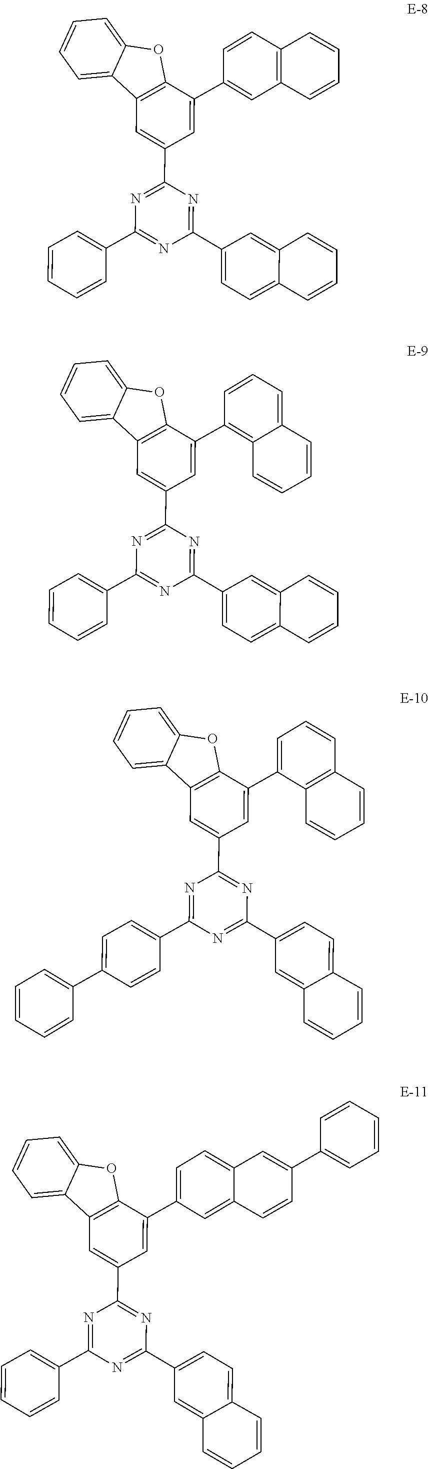 [SMILES: c1ccc(-c2nc(-c3ccc4ccccc4c3)nc(-c3cc(-c4ccc5ccccc5c4)c4oc5ccccc5c4c3)n2)cc1 ; c1ccc(-c2nc(-c3ccc4ccccc4c3)nc(-c3cc(-c4cccc5ccccc45)c4oc5ccccc5c4c3)n2)cc1 ; c1ccc(-c2ccc(-c3nc(-c4ccc5ccccc5c4)nc(-c4cc(-c5cccc6ccccc56)c5oc6ccccc6c5c4)n3)cc2)cc1 ; c1ccc(-c2ccc3cc(-c4cc(-c5nc(-c6ccccc6)nc(-c6ccc7ccccc7c6)n5)cc5c4oc4ccccc45)ccc3c2)cc1]

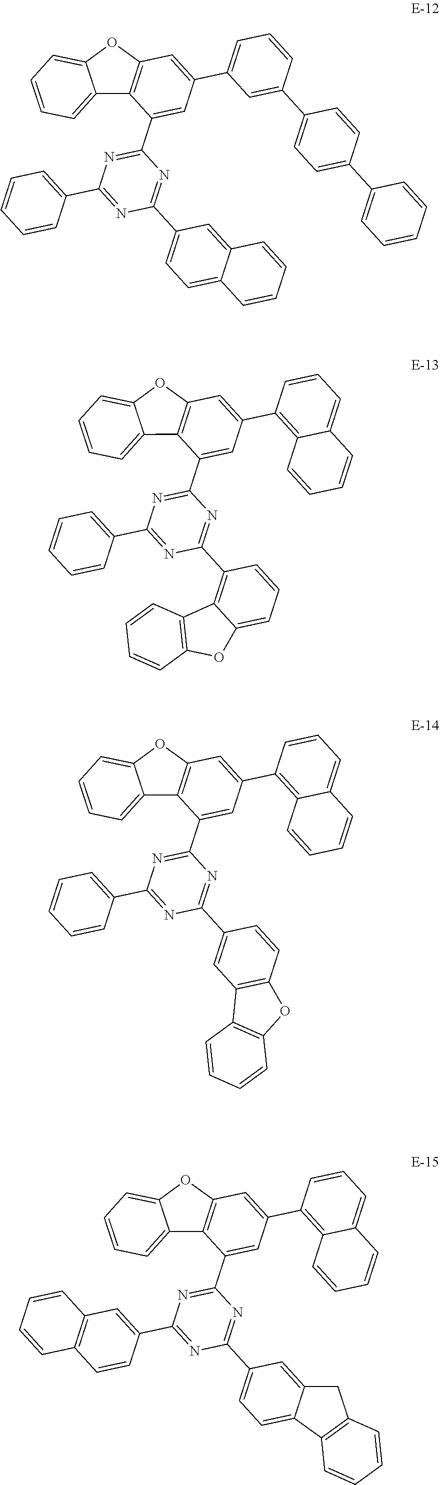 [SMILES: c1ccc(-c2ccc(-c3cccc(-c4cc(-c5nc(-c6ccccc6)nc(-c6ccc7ccccc7c6)n5)c5c(c4)oc4ccccc45)c3)cc2)cc1 ; c1ccc(-c2nc(-c3cccc4oc5ccccc5c34)nc(-c3cc(-c4cccc5ccccc45)cc4oc5ccccc5c34)n2)cc1 ; c1ccc(-c2nc(-c3ccc4oc5ccccc5c4c3)nc(-c3cc(-c4cccc5ccccc45)cc4oc5ccccc5c34)n2)cc1 ; c1ccc2c(c1)Cc1cc(-c3nc(-c4ccc5ccccc5c4)nc(-c4cc(-c5cccc6ccccc56)cc5oc6ccccc6c45)n3)ccc1-2]

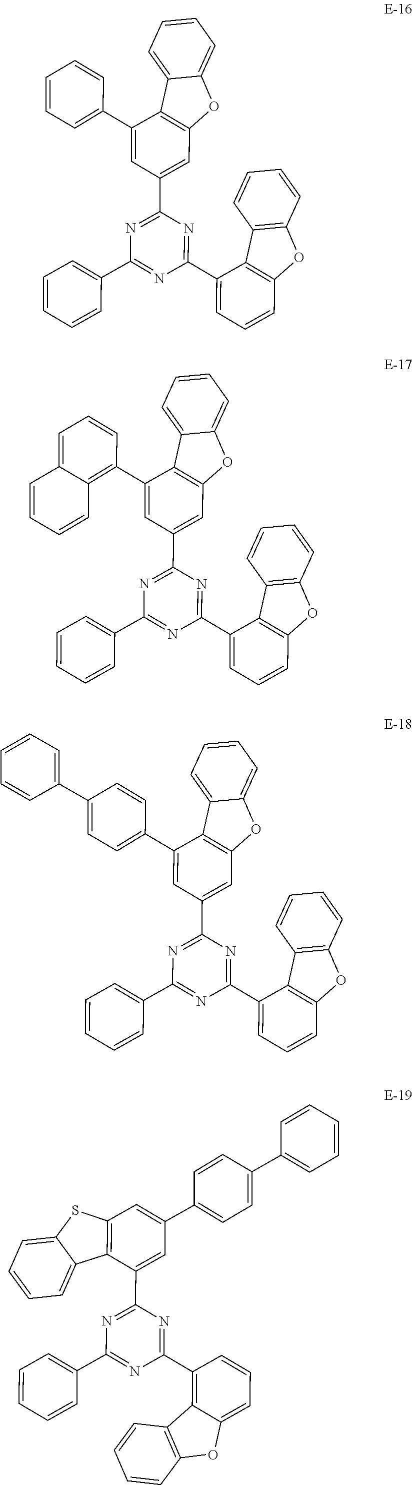 [SMILES: c1ccc(-c2nc(-c3cc(-c4ccccc4)c4c(c3)oc3ccccc34)nc(-c3cccc4oc5ccccc5c34)n2)cc1 ; c1ccc(-c2nc(-c3cc(-c4cccc5ccccc45)c4c(c3)oc3ccccc34)nc(-c3cccc4oc5ccccc5c34)n2)cc1 ; c1ccc(-c2ccc(-c3cc(-c4nc(-c5ccccc5)nc(-c5cccc6oc7ccccc7c56)n4)cc4oc5ccccc5c34)cc2)cc1 ; c1ccc(-c2ccc(-c3cc(-c4nc(-c5ccccc5)nc(-c5cccc6oc7ccccc7c56)n4)c4c(c3)sc3ccccc34)cc2)cc1]

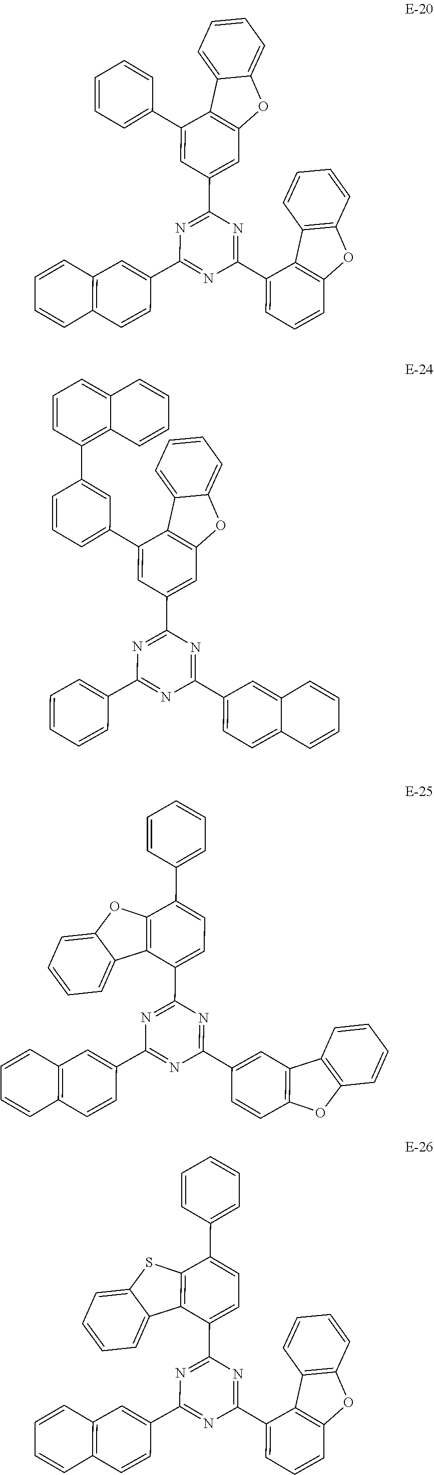 [SMILES: c1ccc(-c2cc(-c3nc(-c4ccc5ccccc5c4)nc(-c4cccc5oc6ccccc6c45)n3)cc3oc4ccccc4c23)cc1 ; c1ccc(-c2nc(-c3ccc4ccccc4c3)nc(-c3cc(-c4cccc(-c5cccc6ccccc56)c4)c4c(c3)oc3ccccc34)n2)cc1 ; c1ccc(-c2ccc(-c3nc(-c4ccc5ccccc5c4)nc(-c4ccc5oc6ccccc6c5c4)n3)c3c2oc2ccccc23)cc1 ; c1ccc(-c2ccc(-c3nc(-c4ccc5ccccc5c4)nc(-c4cccc5oc6ccccc6c45)n3)c3c2sc2ccccc23)cc1]

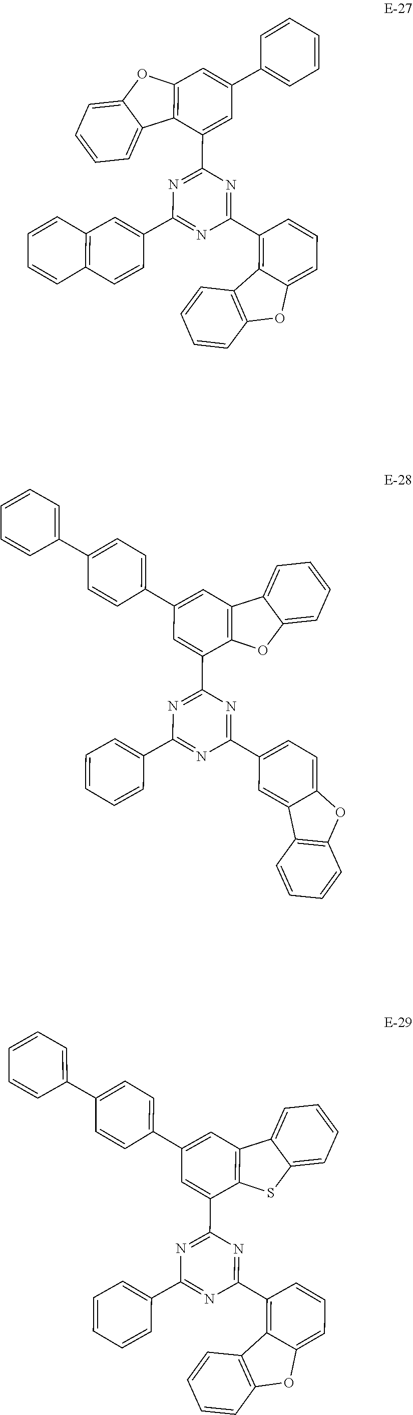 [SMILES: c1ccc(-c2cc(-c3nc(-c4ccc5ccccc5c4)nc(-c4cccc5oc6ccccc6c45)n3)c3c(c2)oc2ccccc23)cc1 ; c1ccc(-c2ccc(-c3cc(-c4nc(-c5ccccc5)nc(-c5ccc6oc7ccccc7c6c5)n4)c4oc5ccccc5c4c3)cc2)cc1 ; c1ccc(-c2ccc(-c3cc(-c4nc(-c5ccccc5)nc(-c5cccc6oc7ccccc7c56)n4)c4sc5ccccc5c4c3)cc2)cc1]

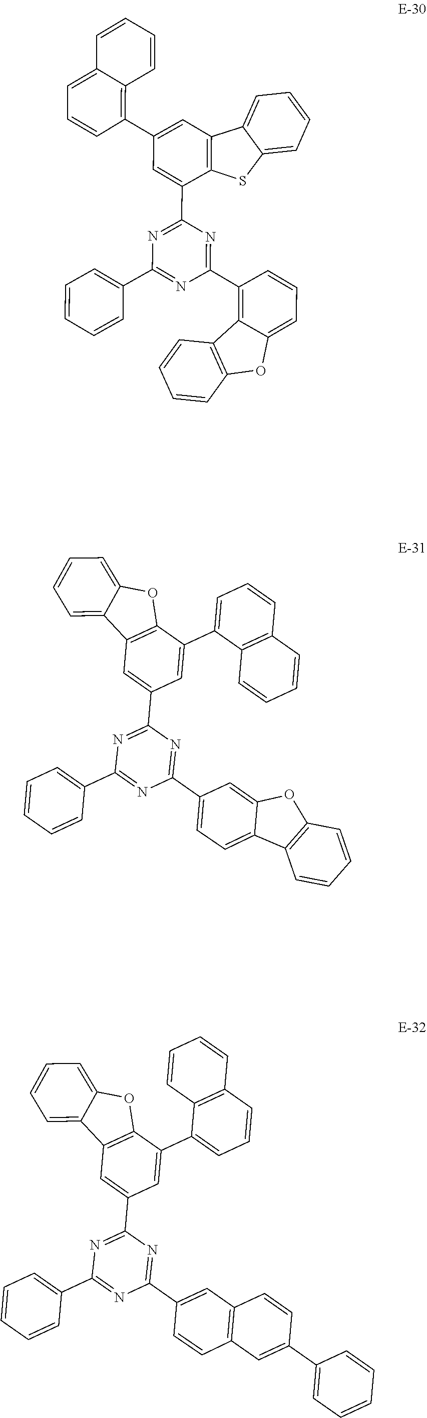 [SMILES: c1ccc(-c2nc(-c3cc(-c4cccc5ccccc45)cc4c3sc3ccccc34)nc(-c3cccc4oc5ccccc5c34)n2)cc1 ; c1ccc(-c2nc(-c3ccc4c(c3)oc3ccccc34)nc(-c3cc(-c4cccc5ccccc45)c4oc5ccccc5c4c3)n2)cc1 ; c1ccc(-c2ccc3cc(-c4nc(-c5ccccc5)nc(-c5cc(-c6cccc7ccccc67)c6oc7ccccc7c6c5)n4)ccc3c2)cc1]

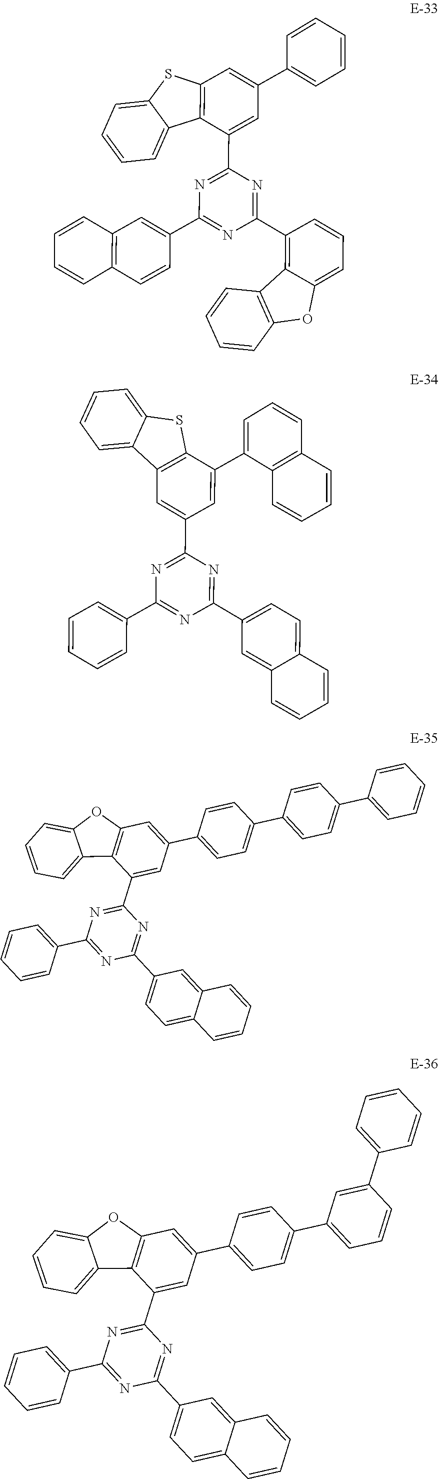 [SMILES: c1ccc(-c2cc(-c3nc(-c4ccc5ccccc5c4)nc(-c4cccc5oc6ccccc6c45)n3)c3c(c2)sc2ccccc23)cc1 ; c1ccc(-c2nc(-c3ccc4ccccc4c3)nc(-c3cc(-c4cccc5ccccc45)c4sc5ccccc5c4c3)n2)cc1 ; c1ccc(-c2ccc(-c3ccc(-c4cc(-c5nc(-c6ccccc6)nc(-c6ccc7ccccc7c6)n5)c5c(c4)oc4ccccc45)cc3)cc2)cc1 ; c1ccc(-c2cccc(-c3ccc(-c4cc(-c5nc(-c6ccccc6)nc(-c6ccc7ccccc7c6)n5)c5c(c4)oc4ccccc45)cc3)c2)cc1]

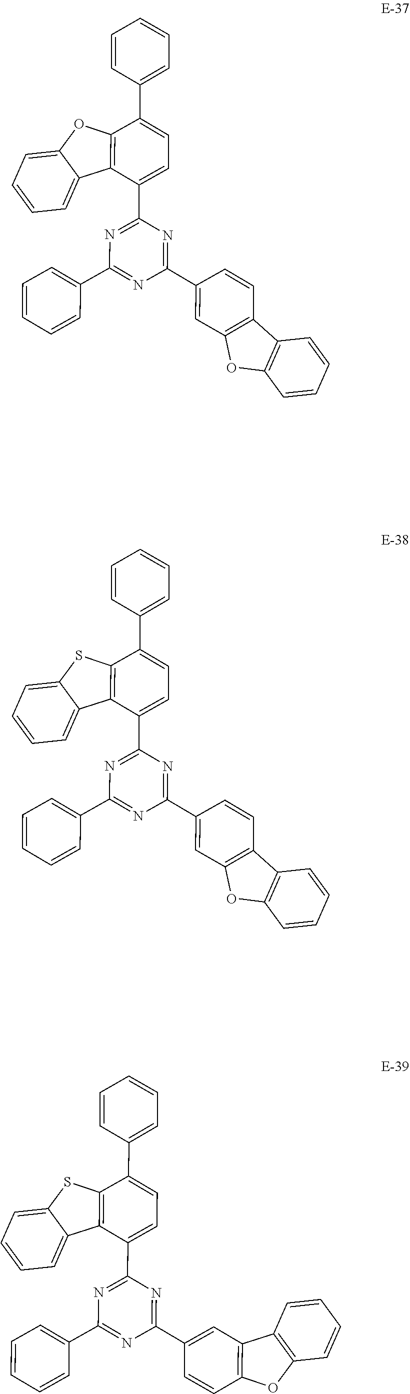 [SMILES: c1ccc(-c2nc(-c3ccc4c(c3)oc3ccccc34)nc(-c3ccc(-c4ccccc4)c4oc5ccccc5c34)n2)cc1 ; c1ccc(-c2nc(-c3ccc4c(c3)oc3ccccc34)nc(-c3ccc(-c4ccccc4)c4sc5ccccc5c34)n2)cc1 ; c1ccc(-c2nc(-c3ccc4oc5ccccc5c4c3)nc(-c3ccc(-c4ccccc4)c4sc5ccccc5c34)n2)cc1]

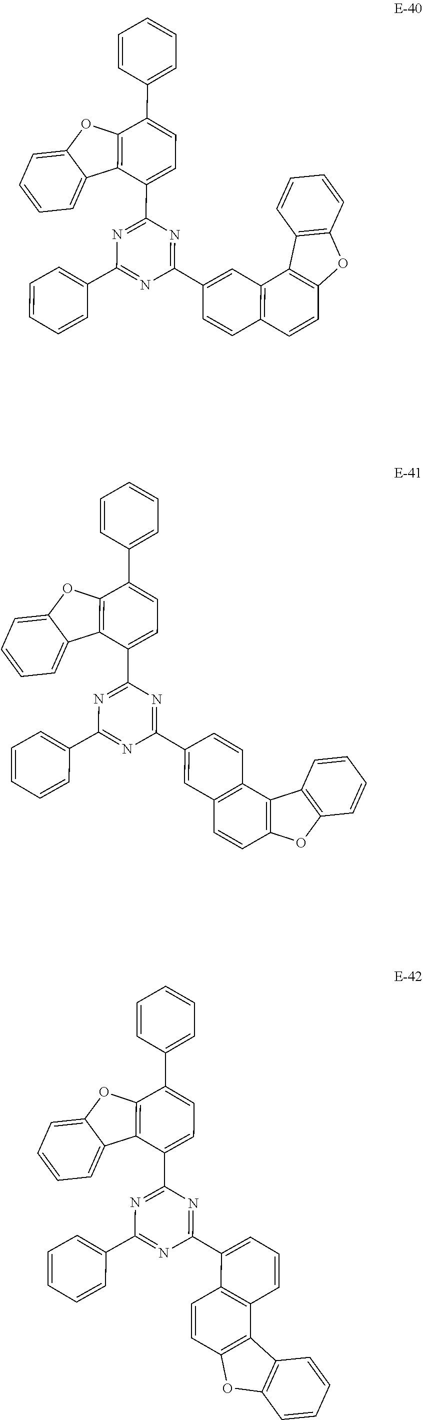 [SMILES: c1ccc(-c2nc(-c3ccc4ccc5oc6ccccc6c5c4c3)nc(-c3ccc(-c4ccccc4)c4oc5ccccc5c34)n2)cc1 ; c1ccc(-c2nc(-c3ccc4c(ccc5oc6ccccc6c54)c3)nc(-c3ccc(-c4ccccc4)c4oc5ccccc5c34)n2)cc1 ; c1ccc(-c2nc(-c3cccc4c3ccc3oc5ccccc5c34)nc(-c3ccc(-c4ccccc4)c4oc5ccccc5c34)n2)cc1]

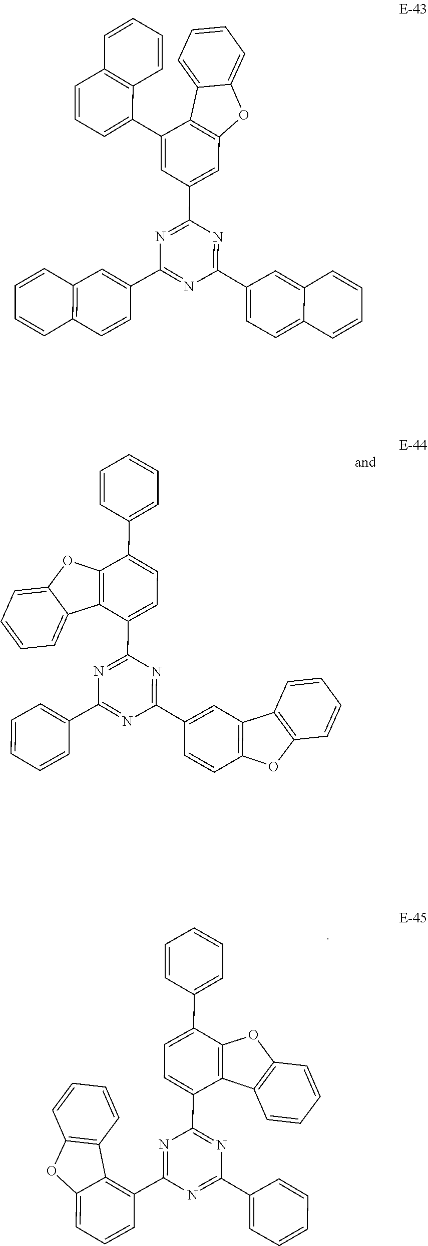 [SMILES: c1ccc2cc(-c3nc(-c4ccc5ccccc5c4)nc(-c4cc(-c5cccc6ccccc56)c5c(c4)oc4ccccc45)n3)ccc2c1 ; c1ccc(-c2nc(-c3ccc4oc5ccccc5c4c3)nc(-c3ccc(-c4ccccc4)c4oc5ccccc5c34)n2)cc1 ; c1ccc(-c2nc(-c3cccc4oc5ccccc5c34)nc(-c3ccc(-c4ccccc4)c4oc5ccccc5c34)n2)cc1]

13. An organic electroluminescent device comprising the organic electroluminescent compound according to claim 11.
